SEQUENCE LISTING (1214 sequences)

SEQ ID NO: 1 - DNA, 20 bp, Artificial Sequence
  Description of Artificial Sequence Synthetic oligonucleotide
gaccaggatgggcaccaccc

SEQ ID NO: 2 - DNA, 21 bp, Artificial Sequence
  Description of Artificial Sequence Synthetic oligonucleotide
gagtaagcagtcctggcaccc

SEQ ID NO: 3 - DNA, 20 bp, Artificial Sequence
  Description of Artificial Sequence Synthetic oligonucleotide
gatgtttagcagccctgccg

SEQ ID NO: 4 - DNA, 20 bp, Artificial Sequence
  Description of Artificial Sequence Synthetic oligonucleotide
gccgatggtcttgtactaca

SEQ ID NO: 5 - DNA, 20 bp, Artificial Sequence
  Description of Artificial Sequence Synthetic oligonucleotide
ggatgtttctgtgctcgtgg

SEQ ID NO: 6 - DNA, 20 bp, Artificial Sequence
  Description of Artificial Sequence Synthetic oligonucleotide
gctgcgtctactgcgaggta

SEQ ID NO: 7 - DNA, 20 bp, Artificial Sequence
  Description of Artificial Sequence Synthetic oligonucleotide
gctgtgctcgtgggggtact

SEQ ID NO: 8 - DNA, 20 bp, Artificial Sequence
  Description of Artificial Sequence Synthetic oligonucleotide
gcggactgcgagtctctttg

SEQ ID NO: 9 - DNA, 20 bp, Artificial Sequence
  Description of Artificial Sequence Synthetic oligonucleotide
ggagactcgcagtccggccc

SEQ ID NO: 10 - DNA, 20 bp, Artificial Sequence
  Description of Artificial Sequence Synthetic oligonucleotide
ggcccgatggcaccttggag

SEQ ID NO: 11 - DNA, 20 bp, Artificial Sequence
  Description of Artificial Sequence Synthetic oligonucleotide
gggaagcaaggaccgaccga

SEQ ID NO: 12 - DNA, 20 bp, Artificial Sequence
  Description of Artificial Sequence Synthetic oligonucleotide
gcgaggcgcggaggcgaagc

SEQ ID NO: 13 - DNA, 20 bp, Artificial Sequence
  Description of Artificial Sequence Synthetic oligonucleotide
ggcaaggaccgaccgacgga

SEQ ID NO: 14 - DNA, 20 bp, Artificial Sequence
  Description of Artificial Sequence Synthetic oligonucleotide
gaggccgggctaggcttagg

SEQ ID NO: 15 - DNA, 20 bp, Artificial Sequence
  Description of Artificial Sequence Synthetic oligonucleotide
ggcgtagtgaccatcatgtg

SEQ ID NO: 16 - DNA, 20 bp, Artificial Sequence
  Description of Artificial Sequence Synthetic oligonucleotide
gctagcccggcctcacatga

SEQ ID NO: 17 - DNA, 20 bp, Artificial Sequence
  Description of Artificial Sequence Synthetic oligonucleotide
gagtacctccatggtcccgg

SEQ ID NO: 18 - DNA, 20 bp, Artificial Sequence
  Description of Artificial Sequence Synthetic oligonucleotide
gacagccaccgggaccatgg

SEQ ID NO: 19 - DNA, 20 bp, Artificial Sequence
  Description of Artificial Sequence Synthetic oligonucleotide
gccagtgacagggcactcag

SEQ ID NO: 20 - DNA, 20 bp, Artificial Sequence
  Description of Artificial Sequence Synthetic oligonucleotide
ggctggagagaagccgggcg

SEQ ID NO: 21 - DNA, 20 bp, Artificial Sequence
  Description of Artificial Sequence Synthetic oligonucleotide
gcgagtacggagaagcggga

SEQ ID NO: 22 - DNA, 20 bp, Artificial Sequence
  Description of Artificial Sequence Synthetic oligonucleotide
gtaggagcctcgggcgcgct

SEQ ID NO: 23 - DNA, 20 bp, Artificial Sequence
  Description of Artificial Sequence Synthetic oligonucleotide
gcggcaggaaggttctgaga

SEQ ID NO: 24 - DNA, 20 bp, Artificial Sequence
  Description of Artificial Sequence Synthetic oligonucleotide
gaggttggaatgcgccccag

SEQ ID NO: 25 - DNA, 20 bp, Artificial Sequence
  Description of Artificial Sequence Synthetic oligonucleotide
gcagcgaggtgcaaacgcga

SEQ ID NO: 26 - DNA, 20 bp, Artificial Sequence
  Description of Artificial Sequence Synthetic oligonucleotide
ggtgcacgtactcccaactg

SEQ ID NO: 27 - DNA, 20 bp, Artificial Sequence
  Description of Artificial Sequence Synthetic oligonucleotide
gtgagagcgcgaccacagtt

SEQ ID NO: 28 - DNA, 20 bp, Artificial Sequence
  Description of Artificial Sequence Synthetic oligonucleotide
ggggccacgagagcagcaga

SEQ ID NO: 29 - DNA, 20 bp, Artificial Sequence
  Description of Artificial Sequence Synthetic oligonucleotide
gaggcgagcgaggagaccac

SEQ ID NO: 30 - DNA, 20 bp, Artificial Sequence
  Description of Artificial Sequence Synthetic oligonucleotide
gcgtctggaaggaatccacg

SEQ ID NO: 31 - DNA, 20 bp, Artificial Sequence
  Description of Artificial Sequence Synthetic oligonucleotide
gcaggacgggcgcgtcttca

SEQ ID NO: 32 - DNA, 20 bp, Artificial Sequence
  Description of Artificial Sequence Synthetic oligonucleotide
gggatgagccgtgctcccgg

SEQ ID NO: 33 - DNA, 20 bp, Artificial Sequence
  Description of Artificial Sequence Synthetic oligonucleotide
gcttgcctggaactcctgta

SEQ ID NO: 34 - DNA, 20 bp, Artificial Sequence
  Description of Artificial Sequence Synthetic oligonucleotide
gggcgcctgtgcttgaggtt

SEQ ID NO: 35 - DNA, 20 bp, Artificial Sequence
  Description of Artificial Sequence Synthetic oligonucleotide
gtggccgttcatgggaccgg

SEQ ID NO: 36 - DNA, 20 bp, Artificial Sequence
  Description of Artificial Sequence Synthetic oligonucleotide
gacaatatctggatcgccaa

SEQ ID NO: 37 - DNA, 20 bp, Artificial Sequence
  Description of Artificial Sequence Synthetic oligonucleotide
ggatgggctcgcctggccag

SEQ ID NO: 38 - DNA, 20 bp, Artificial Sequence
  Description of Artificial Sequence Synthetic oligonucleotide
ggtcccacggcgaagcgact

SEQ ID NO: 39 - DNA, 20 bp, Artificial Sequence
  Description of Artificial Sequence Synthetic oligonucleotide
ggcgcgggccccataaaaac

SEQ ID NO: 40 - DNA, 20 bp, Artificial Sequence
  Description of Artificial Sequence Synthetic oligonucleotide
gctgcgatgccgctggatca

SEQ ID NO: 41 - DNA, 20 bp, Artificial Sequence
  Description of Artificial Sequence Synthetic oligonucleotide
gctgcaatgccgctggatca

SEQ ID NO: 42 - DNA, 20 bp, Artificial Sequence
  Description of Artificial Sequence Synthetic oligonucleotide
gctgggatgccgctggatca

SEQ ID NO: 43 - DNA, 20 bp, Artificial Sequence
  Description of Artificial Sequence Synthetic oligonucleotide
gctgcgattccgctggatca

SEQ ID NO: 44 - DNA, 20 bp, Artificial Sequence
  Description of Artificial Sequence Synthetic oligonucleotide
gctgcgatcccgctggatca

SEQ ID NO: 45 - DNA, 20 bp, Artificial Sequence
  Description of Artificial Sequence Synthetic oligonucleotide
gaggcccgggcgcgacaatc

SEQ ID NO: 46 - DNA, 20 bp, Artificial Sequence
  Description of Artificial Sequence Synthetic oligonucleotide
gagacccgggcgcgacaatc

SEQ ID NO: 47 - DNA, 20 bp, Artificial Sequence
  Description of Artificial Sequence Synthetic oligonucleotide
gaggccctggcgcgacaatc

SEQ ID NO: 48 - DNA, 20 bp, Artificial Sequence
  Description of Artificial Sequence Synthetic oligonucleotide
gaggcccgcgcgcgacaatc

SEQ ID NO: 49 - DNA, 20 bp, Artificial Sequence
  Description of Artificial Sequence Synthetic oligonucleotide
gaggcccgggcgcgacagtc

SEQ ID NO: 50 - DNA, 20 bp, Artificial Sequence
  Description of Artificial Sequence Synthetic oligonucleotide
gaggcccgggctcgacaatc

SEQ ID NO: 51 - DNA, 20 bp, Artificial Sequence
  Description of Artificial Sequence Synthetic oligonucleotide
ggatgattctgtgctcgtgg

SEQ ID NO: 52 - DNA, 20 bp, Artificial Sequence
  Description of Artificial Sequence Synthetic oligonucleotide
ggatgtttcggtgctcgtgg

SEQ ID NO: 53 - DNA, 20 bp, Artificial Sequence
  Description of Artificial Sequence Synthetic oligonucleotide
ggatgtttcagtgctcgtgg

SEQ ID NO: 54 - DNA, 20 bp, Artificial Sequence
  Description of Artificial Sequence Synthetic oligonucleotide
ggatgtttctgcgctcgtgg

SEQ ID NO: 55 - DNA, 20 bp, Artificial Sequence
  Description of Artificial Sequence Synthetic oligonucleotide
gtgcaaggcggcggcaggag

SEQ ID NO: 56 - DNA, 20 bp, Artificial Sequence
  Description of Artificial Sequence Synthetic oligonucleotide
gtgcaaggtggcggcaggag

SEQ ID NO: 57 - DNA, 20 bp, Artificial Sequence
  Description of Artificial Sequence Synthetic oligonucleotide
gtgcaaggcggcggcgggag

SEQ ID NO: 58 - DNA, 20 bp, Artificial Sequence
  Description of Artificial Sequence Synthetic oligonucleotide
gtgcaaggcgggggcaggag

SEQ ID NO: 59 - DNA, 20 bp, Artificial Sequence
  Description of Artificial Sequence Synthetic oligonucleotide
gtgcaagacggcggcaggag

SEQ ID NO: 60 - DNA, 20 bp, Artificial Sequence
  Description of Artificial Sequence Synthetic oligonucleotide
gaccaaagcgatctcttctg

SEQ ID NO: 61 - DNA, 20 bp, Artificial Sequence
  Description of Artificial Sequence Synthetic oligonucleotide
gaccaaagcggtctcttctg

SEQ ID NO: 62 - DNA, 20 bp, Artificial Sequence
  Description of Artificial Sequence Synthetic oligonucleotide
gaccaaggcgatctcttctg

SEQ ID NO: 63 - DNA, 20 bp, Artificial Sequence
  Description of Artificial Sequence Synthetic oligonucleotide
gaccaaagcgatctcttgtg

SEQ ID NO: 64 - DNA, 20 bp, Artificial Sequence
  Description of Artificial Sequence Synthetic oligonucleotide
gaccaaaccgatctcttctg

SEQ ID NO: 65 - DNA, 20 bp, Artificial Sequence
  Description of Artificial Sequence Synthetic oligonucleotide
ggagacacgcagtccggccc

SEQ ID NO: 66 - DNA, 20 bp, Artificial Sequence
  Description of Artificial Sequence Synthetic oligonucleotide
ggtgactcgcagtccggccc

SEQ ID NO: 67 - DNA, 20 bp, Artificial Sequence
  Description of Artificial Sequence Synthetic oligonucleotide
ggagactggcagtccggccc

SEQ ID NO: 68 - DNA, 20 bp, Artificial Sequence
  Description of Artificial Sequence Synthetic oligonucleotide
ggagactcgcagtcctgccc

SEQ ID NO: 69 - DNA, 20 bp, Artificial Sequence
  Description of Artificial Sequence Synthetic oligonucleotide
ggcggtcgctgcgcttaggg

SEQ ID NO: 70 - DNA, 20 bp, Artificial Sequence
  Description of Artificial Sequence Synthetic oligonucleotide
ggcggccgctgcgcttaggg

SEQ ID NO: 71 - DNA, 20 bp, Artificial Sequence
  Description of Artificial Sequence Synthetic oligonucleotide
gggggtcgctgcgcttaggg

SEQ ID NO: 72 - DNA, 20 bp, Artificial Sequence
  Description of Artificial Sequence Synthetic oligonucleotide
ggcggtcgcggcgcttaggg

SEQ ID NO: 73 - DNA, 20 bp, Artificial Sequence
  Description of Artificial Sequence Synthetic oligonucleotide
ggcggtcgctgcgcttaggt

SEQ ID NO: 74 - DNA, 20 bp, Artificial Sequence
  Description of Artificial Sequence Synthetic oligonucleotide
gggaagcagggaccgaccga

SEQ ID NO: 75 - DNA, 20 bp, Artificial Sequence
  Description of Artificial Sequence Synthetic oligonucleotide
ggtaagcaaggaccgaccga

SEQ ID NO: 76 - DNA, 20 bp, Artificial Sequence
  Description of Artificial Sequence Synthetic oligonucleotide
gggaagccaggaccgaccga

SEQ ID NO: 77 - DNA, 20 bp, Artificial Sequence
  Description of Artificial Sequence Synthetic oligonucleotide
gggaagcaaggagcgaccga

SEQ ID NO: 78 - DNA, 20 bp, Artificial Sequence
  Description of Artificial Sequence Synthetic oligonucleotide
gtttgcaggagtctacaccc

SEQ ID NO: 79 - DNA, 20 bp, Artificial Sequence
  Description of Artificial Sequence Synthetic oligonucleotide
gattgcaggagtctacaccc

SEQ ID NO: 80 - DNA, 20 bp, Artificial Sequence
  Description of Artificial Sequence Synthetic oligonucleotide
gtttgcaggggtctacaccc

SEQ ID NO: 81 - DNA, 20 bp, Artificial Sequence
  Description of Artificial Sequence Synthetic oligonucleotide
gtttgcaggagtgtacaccc

SEQ ID NO: 82 - DNA, 20 bp, Artificial Sequence
  Description of Artificial Sequence Synthetic oligonucleotide
gtttgcagtagtctacaccc

SEQ ID NO: 83 - DNA, 20 bp, Artificial Sequence
  Description of Artificial Sequence Synthetic oligonucleotide
ggcactgacgtgtctctcgg

SEQ ID NO: 84 - DNA, 20 bp, Artificial Sequence
  Description of Artificial Sequence Synthetic oligonucleotide
ggcgctgacgtgtctctcgg

SEQ ID NO: 85 - DNA, 20 bp, Artificial Sequence
  Description of Artificial Sequence Synthetic oligonucleotide
ggcactgtcgtgtctctcgg

SEQ ID NO: 86 - DNA, 20 bp, Artificial Sequence
  Description of Artificial Sequence Synthetic oligonucleotide
ggtactgacgtgtctctcgg

SEQ ID NO: 87 - DNA, 20 bp, Artificial Sequence
  Description of Artificial Sequence Synthetic oligonucleotide
ggcactgaagtgtctctcgg

SEQ ID NO: 88 - DNA, 20 bp, Artificial Sequence
  Description of Artificial Sequence Synthetic oligonucleotide
gagccgagtaggcgacggtg

SEQ ID NO: 89 - DNA, 20 bp, Artificial Sequence
  Description of Artificial Sequence Synthetic oligonucleotide
gagccgagaaggcgacggtg

SEQ ID NO: 90 - DNA, 20 bp, Artificial Sequence
  Description of Artificial Sequence Synthetic oligonucleotide
gagccgagtgggcgacggtg

SEQ ID NO: 91 - DNA, 20 bp, Artificial Sequence
  Description of Artificial Sequence Synthetic oligonucleotide
gaaccgagtaggcgacggtg

SEQ ID NO: 92 - DNA, 20 bp, Artificial Sequence
  Description of Artificial Sequence Synthetic oligonucleotide
gagccgagtagacgacggtg

SEQ ID NO: 93 - DNA, 20 bp, Artificial Sequence
  Description of Artificial Sequence Synthetic oligonucleotide
ggactagaacgaaaggagtg

SEQ ID NO: 94 - DNA, 20 bp, Artificial Sequence
  Description of Artificial Sequence Synthetic oligonucleotide
ggactagagcgaaaggagtg

SEQ ID NO: 95 - DNA, 20 bp, Artificial Sequence
  Description of Artificial Sequence Synthetic oligonucleotide
ggactagaacggaaggagtg

SEQ ID NO: 96 - DNA, 20 bp, Artificial Sequence
  Description of Artificial Sequence Synthetic oligonucleotide
ggactataacgaaaggagtg

SEQ ID NO: 97 - DNA, 20 bp, Artificial Sequence
  Description of Artificial Sequence Synthetic oligonucleotide
ggactagaacgaaaggagcg

SEQ ID NO: 98 - DNA, 20 bp, Artificial Sequence
  Description of Artificial Sequence Synthetic oligonucleotide
gacagctaccgggaccatgg

SEQ ID NO: 99 - DNA, 20 bp, Artificial Sequence
  Description of Artificial Sequence Synthetic oligonucleotide
gacagccatcgggaccatgg

SEQ ID NO: 100 - DNA, 20 bp, Artificial Sequence
  Description of Artificial Sequence Synthetic oligonucleotide
gacagccaacgggaccatgg

SEQ ID NO: 101 - DNA, 20 bp, Artificial Sequence
  Description of Artificial Sequence Synthetic oligonucleotide
gacagccaccgggaccacgg

SEQ ID NO: 102 - DNA, 20 bp, Artificial Sequence
  Description of Artificial Sequence Synthetic oligonucleotide
ggctggtgagaagccgggcg

SEQ ID NO: 103 - DNA, 20 bp, Artificial Sequence
  Description of Artificial Sequence Synthetic oligonucleotide
ggctggagcgaagccgggcg

SEQ ID NO: 104 - DNA, 20 bp, Artificial Sequence
  Description of Artificial Sequence Synthetic oligonucleotide
ggctggagagtagccgggcg

SEQ ID NO: 105 - DNA, 20 bp, Artificial Sequence
  Description of Artificial Sequence Synthetic oligonucleotide
ggctggagagaagcctggcg

SEQ ID NO: 106 - DNA, 20 bp, Artificial Sequence
  Description of Artificial Sequence Synthetic oligonucleotide
gcagcaggaaggttctgaga

SEQ ID NO: 107 - DNA, 20 bp, Artificial Sequence
  Description of Artificial Sequence Synthetic oligonucleotide
gcggcaggacggttctgaga

SEQ ID NO: 108 - DNA, 20 bp, Artificial Sequence
  Description of Artificial Sequence Synthetic oligonucleotide
gcggcagcaaggttctgaga

SEQ ID NO: 109 - DNA, 20 bp, Artificial Sequence
  Description of Artificial Sequence Synthetic oligonucleotide
gcggcaggaaggttcagaga

SEQ ID NO: 110 - DNA, 20 bp, Artificial Sequence
  Description of Artificial Sequence Synthetic oligonucleotide
gcgagcgaggagaccaccgg

SEQ ID NO: 111 - DNA, 20 bp, Artificial Sequence
  Description of Artificial Sequence Synthetic oligonucleotide
gccagcgaggagaccaccgg

SEQ ID NO: 112 - DNA, 20 bp, Artificial Sequence
  Description of Artificial Sequence Synthetic oligonucleotide
gcgagcgaggaggccaccgg

SEQ ID NO: 113 - DNA, 20 bp, Artificial Sequence
  Description of Artificial Sequence Synthetic oligonucleotide
gcgagcgaggagcccaccgg

SEQ ID NO: 114 - DNA, 20 bp, Artificial Sequence
  Description of Artificial Sequence Synthetic oligonucleotide
gcgagcgaggagaccaacgg

SEQ ID NO: 115 - DNA, 20 bp, Artificial Sequence
  Description of Artificial Sequence Synthetic oligonucleotide
ggggccacgagagcagcgga

SEQ ID NO: 116 - DNA, 20 bp, Artificial Sequence
  Description of Artificial Sequence Synthetic oligonucleotide
ggggccacgcgagcagcaga

SEQ ID NO: 117 - DNA, 20 bp, Artificial Sequence
  Description of Artificial Sequence Synthetic oligonucleotide
ggggccacgagagcaggaga

SEQ ID NO: 118 - DNA, 20 bp, Artificial Sequence
  Description of Artificial Sequence Synthetic oligonucleotide
ggggccacgagtgcagcaga

SEQ ID NO: 119 - DNA, 20 bp, Artificial Sequence
  Description of Artificial Sequence Synthetic oligonucleotide
gtgagcttgccacatcccca

SEQ ID NO: 120 - DNA, 20 bp, Artificial Sequence
  Description of Artificial Sequence Synthetic oligonucleotide
gtgcgcttgccacatcccca

SEQ ID NO: 121 - DNA, 20 bp, Artificial Sequence
  Description of Artificial Sequence Synthetic oligonucleotide
gtgagcttaccacatcccca

SEQ ID NO: 122 - DNA, 20 bp, Artificial Sequence
  Description of Artificial Sequence Synthetic oligonucleotide
gtgagctttccacatcccca

SEQ ID NO: 123 - DNA, 20 bp, Artificial Sequence
  Description of Artificial Sequence Synthetic oligonucleotide
gtgagcttgcgacatcccca

SEQ ID NO: 124 - DNA, 20 bp, Artificial Sequence
  Description of Artificial Sequence Synthetic oligonucleotide
gtgagcttgccacatccgca

SEQ ID NO: 125 - DNA, 20 bp, Artificial Sequence
  Description of Artificial Sequence Synthetic oligonucleotide
gcgcgcggggcccgtctcag

SEQ ID NO: 126 - DNA, 20 bp, Artificial Sequence
  Description of Artificial Sequence Synthetic oligonucleotide
gcgcgcggggctcgtctcag

SEQ ID NO: 127 - DNA, 20 bp, Artificial Sequence
  Description of Artificial Sequence Synthetic oligonucleotide
gcgcgcggagcccgtctcag

SEQ ID NO: 128 - DNA, 20 bp, Artificial Sequence
  Description of Artificial Sequence Synthetic oligonucleotide
gcgcgcggcgcccgtctcag

SEQ ID NO: 129 - DNA, 20 bp, Artificial Sequence
  Description of Artificial Sequence Synthetic oligonucleotide
gcgcgcggggcccgtcgcag

SEQ ID NO: 130 - DNA, 20 bp, Artificial Sequence
  Description of Artificial Sequence Synthetic oligonucleotide
gcgcgcggggcccatctcag

SEQ ID NO: 131 - DNA, 20 bp, Artificial Sequence
  Description of Artificial Sequence Synthetic oligonucleotide
ggagctgcgcgatgcggtgg

SEQ ID NO: 132 - DNA, 20 bp, Artificial Sequence
  Description of Artificial Sequence Synthetic oligonucleotide
ggagctgcgggatgcggtgg

SEQ ID NO: 133 - DNA, 20 bp, Artificial Sequence
  Description of Artificial Sequence Synthetic oligonucleotide
ggagttgcgcgatgcggtgg

SEQ ID NO: 134 - DNA, 20 bp, Artificial Sequence
  Description of Artificial Sequence Synthetic oligonucleotide
ggagctgctcgatgcggtgg

SEQ ID NO: 135 - DNA, 20 bp, Artificial Sequence
  Description of Artificial Sequence Synthetic oligonucleotide
ggagctgcgcaatgcggtgg

SEQ ID NO: 136 - DNA, 20 bp, Artificial Sequence
  Description of Artificial Sequence Synthetic oligonucleotide
gagcgaagcgcacgctgagg

SEQ ID NO: 137 - DNA, 20 bp, Artificial Sequence
  Description of Artificial Sequence Synthetic oligonucleotide
gagcgaagcgcgcgctgagg

SEQ ID NO: 138 - DNA, 20 bp, Artificial Sequence
  Description of Artificial Sequence Synthetic oligonucleotide
gagcgcagcgcacgctgagg

SEQ ID NO: 139 - DNA, 20 bp, Artificial Sequence
  Description of Artificial Sequence Synthetic oligonucleotide
gagcgaaacgcacgctgagg

SEQ ID NO: 140 - DNA, 20 bp, Artificial Sequence
  Description of Artificial Sequence Synthetic oligonucleotide
gagcgaagcgctcgctgagg

SEQ ID NO: 141 - DNA, 20 bp, Artificial Sequence
  Description of Artificial Sequence Synthetic oligonucleotide
ggctctggcgtcctggatgg

SEQ ID NO: 142 - DNA, 20 bp, Artificial Sequence
  Description of Artificial Sequence Synthetic oligonucleotide
ggctctgtcgtcctggatgg

SEQ ID NO: 143 - DNA, 20 bp, Artificial Sequence
  Description of Artificial Sequence Synthetic oligonucleotide
ggctctggcgccctggatgg

SEQ ID NO: 144 - DNA, 20 bp, Artificial Sequence
  Description of Artificial Sequence Synthetic oligonucleotide
ggctctggcgtcttggatgg

SEQ ID NO: 145 - DNA, 20 bp, Artificial Sequence
  Description of Artificial Sequence Synthetic oligonucleotide
ggctctggcgtcccggatgg

SEQ ID NO: 146 - DNA, 20 bp, Artificial Sequence
  Description of Artificial Sequence Synthetic oligonucleotide
gggcagggggcctgaagcgg

SEQ ID NO: 147 - DNA, 20 bp, Artificial Sequence
  Description of Artificial Sequence Synthetic oligonucleotide
gggcagggggtctgaagcgg

SEQ ID NO: 148 - DNA, 20 bp, Artificial Sequence
  Description of Artificial Sequence Synthetic oligonucleotide
gggcagggggcttgaagcgg

SEQ ID NO: 149 - DNA, 20 bp, Artificial Sequence
  Description of Artificial Sequence Synthetic oligonucleotide
gggcaggggggctgaagcgg

SEQ ID NO: 150 - DNA, 20 bp, Artificial Sequence
  Description of Artificial Sequence Synthetic oligonucleotide
gggcagggggcctgaagcag

SEQ ID NO: 151 - DNA, 20 bp, Artificial Sequence
  Description of Artificial Sequence Synthetic oligonucleotide
ggtgtccaggggcactctgt

SEQ ID NO: 152 - DNA, 20 bp, Artificial Sequence
  Description of Artificial Sequence Synthetic oligonucleotide
ggtgtcctggggcactctgt

SEQ ID NO: 153 - DNA, 20 bp, Artificial Sequence
  Description of Artificial Sequence Synthetic oligonucleotide
ggtgtccaggggcgctctgt

SEQ ID NO: 154 - DNA, 20 bp, Artificial Sequence
  Description of Artificial Sequence Synthetic oligonucleotide
ggtgtccaggagcactctgt

SEQ ID NO: 155 - DNA, 20 bp, Artificial Sequence
  Description of Artificial Sequence Synthetic oligonucleotide
ggtgtccaggggcactgtgt

SEQ ID NO: 156 - DNA, 20 bp, Artificial Sequence
  Description of Artificial Sequence Synthetic oligonucleotide
ggtcaccaggaccccttctg

SEQ ID NO: 157 - DNA, 20 bp, Artificial Sequence
  Description of Artificial Sequence Synthetic oligonucleotide
ggtcaccaggatcccttctg

SEQ ID NO: 158 - DNA, 20 bp, Artificial Sequence
  Description of Artificial Sequence Synthetic oligonucleotide
ggtcaccaggcccccttctg

SEQ ID NO: 159 - DNA, 20 bp, Artificial Sequence
  Description of Artificial Sequence Synthetic oligonucleotide
ggtcaccaggaccgcttctg

SEQ ID NO: 160 - DNA, 20 bp, Artificial Sequence
  Description of Artificial Sequence Synthetic oligonucleotide
ggtcaccaggaccccttttg

SEQ ID NO: 161 - DNA, 20 bp, Artificial Sequence
  Description of Artificial Sequence Synthetic oligonucleotide
gtgcacccggctaggaccgg

SEQ ID NO: 162 - DNA, 20 bp, Artificial Sequence
  Description of Artificial Sequence Synthetic oligonucleotide
ggtggcctttgcaattggcg

SEQ ID NO: 163 - DNA, 20 bp, Artificial Sequence
  Description of Artificial Sequence Synthetic oligonucleotide
gggcctggacgagcctaaaa

SEQ ID NO: 164 - DNA, 20 bp, Artificial Sequence
  Description of Artificial Sequence Synthetic oligonucleotide
ggggtgagggtccaattcgg

SEQ ID NO: 165 - DNA, 20 bp, Artificial Sequence
  Description of Artificial Sequence Synthetic oligonucleotide
gtgaactcaaaaatcccgac

SEQ ID NO: 166 - DNA, 20 bp, Artificial Sequence
  Description of Artificial Sequence Synthetic oligonucleotide
gggccgacggataggaggga

SEQ ID NO: 167 - DNA, 20 bp, Artificial Sequence
  Description of Artificial Sequence Synthetic oligonucleotide
ggcgccggactggacctcga

SEQ ID NO: 168 - DNA, 20 bp, Artificial Sequence
  Description of Artificial Sequence Synthetic oligonucleotide
gtgggagcagatcaagactc

SEQ ID NO: 169 - DNA, 20 bp, Artificial Sequence
  Description of Artificial Sequence Synthetic oligonucleotide
gcacgacgctccggcacgcg

SEQ ID NO: 170 - DNA, 20 bp, Artificial Sequence
  Description of Artificial Sequence Synthetic oligonucleotide
gtacggcatggcgcactgcg

SEQ ID NO: 171 - DNA, 84 bp, Artificial Sequence
  Description of Artificial Sequence Synthetic oligonucleotide
taagctggaaacagcatagcaagctcaaataagactagttcgttatcaacttgaaaaagtggcaccgagtcggtgctttttttc

SEQ ID NO: 172 - DNA, 85 bp, Artificial Sequence
  Description of Artificial Sequence Synthetic oligonucleotide
tcgagaaaaaaagcaccgactcggtgccactttttcaagttgataacgaactagtcttatttgagcttgctatgctgtttccagc

SEQ ID NO: 173 - DNA, 84 bp, Artificial Sequence
  Description of Artificial Sequence Synthetic oligonucleotide
taagctggaaacagcatagcaagttcaaataaggctagtccgttatgtacttcaaaaagtggcaccgagtcggtgctttttttc

SEQ ID NO: 174 - DNA, 85 bp, Artificial Sequence
  Description of Artificial Sequence Synthetic oligonucleotide
tcgagaaaaaaagcaccgactcggtgccactttttgaagtacataacggactagccttatttgaacttgctatgctgtttccagc

SEQ ID NO: 175 - DNA, 84 bp, Artificial Sequence
  Description of Artificial Sequence Synthetic oligonucleotide
taagctggaaacagcatagcgagttcaaataaggctcgtccgttatccacttgaaaaagtggcaccgagtcggtgctttttttc

SEQ ID NO: 176 - DNA, 85 bp, Artificial Sequence
  Description of Artificial Sequence Synthetic oligonucleotide
tcgagaaaaaaagcaccgactcggtgccactttttcaagtggataacggacgagccttatttgaactcgctatgctgtttccagc

SEQ ID NO: 177 - DNA, 84 bp, Artificial Sequence
  Description of Artificial Sequence Synthetic oligonucleotide
taagctggaaacagcatagcaagttcaaataaagttaatctgttatcaactcgaaagagtggcaccgagtcggtgctttttttc

SEQ ID NO: 178 - DNA, 85 bp, Artificial Sequence
  Description of Artificial Sequence Synthetic oligonucleotide
tcgagaaaaaaagcaccgactcggtgccactctttcgagttgataacagattaactttatttgaacttgctatgctgtttccagc

SEQ ID NO: 179 - DNA, 84 bp, Artificial Sequence
  Description of Artificial Sequence Synthetic oligonucleotide
taagctggaaacagcatagcaagttcaaataaggctagcccgttatgaacttgaaaaagtggcaccgagtcggtgctttttttc

SEQ ID NO: 180 - DNA, 85 bp, Artificial Sequence
  Description of Artificial Sequence Synthetic oligonucleotide
tcgagaaaaaaagcaccgactcggtgccactttttcaagttcataacgggctagccttatttgaacttgctatgctgtttccagc

SEQ ID NO: 181 - DNA, 84 bp, Artificial Sequence
  Description of Artificial Sequence Synthetic oligonucleotide
taagctggaaacagcatagcaagttcaaataaggctagtccgttatcaacttgaaaaagtggcaccggggcggtgctttttttc

SEQ ID NO: 182 - DNA, 85 bp, Artificial Sequence
  Description of Artificial Sequence Synthetic oligonucleotide
tcgagaaaaaaagcaccgccccggtgccactttttcaagttgataacggactagccttatttgaacttgctatgctgtttccagc

SEQ ID NO: 183 - DNA, 84 bp, Artificial Sequence
  Description of Artificial Sequence Synthetic oligonucleotide
taagctggaaacagcatagcaagttcaaatatggctagtccgttatcaacttgaaaaagtggcaccgagtcggtgctttttttc

SEQ ID NO: 184 - DNA, 85 bp, Artificial Sequence
  Description of Artificial Sequence Synthetic oligonucleotide
tcgagaaaaaaagcaccgactcggtgccactttttcaagttgataacggactagccatatttgaacttgctatgctgtttccagc

SEQ ID NO: 185 - DNA, 84 bp, Artificial Sequence
  Description of Artificial Sequence Synthetic oligonucleotide
taagctggaaacagcatagcaagttcaaataaggatattccgttatcaagttgaaaaactggcaccgagtcggtgctttttttc

SEQ ID NO: 186 - DNA, 85 bp, Artificial Sequence
  Description of Artificial Sequence Synthetic oligonucleotide
tcgagaaaaaaagcaccgactcggtgccagtttttcaacttgataacggaatatccttatttgaacttgctatgctgtttccagc

SEQ ID NO: 187 - DNA, 84 bp, Artificial Sequence
  Description of Artificial Sequence Synthetic oligonucleotide
taagctggaaacagcatagcaagttcaaataaggctagtccgttatcaacttgagaaagtggcaccgggtcggtgctttttttc

SEQ ID NO: 188 - DNA, 85 bp, Artificial Sequence
  Description of Artificial Sequence Synthetic oligonucleotide
tcgagaaaaaaagcaccgacccggtgccactttctcaagttgataacggactagccttatttgaacttgctatgctgtttccagc

SEQ ID NO: 189 - DNA, 84 bp, Artificial Sequence
  Description of Artificial Sequence Synthetic oligonucleotide
taagctggaaacagcatagcaagttcaaataaggctagtccgttatcaacttgaaaaagtggcaccgcgtcggtgctttttttc

SEQ ID NO: 190 - DNA, 85 bp, Artificial Sequence
  Description of Artificial Sequence Synthetic oligonucleotide
tcgagaaaaaaagcaccgacgcggtgccactttttcaagttgataacggactagccttatttgaacttgctatgctgtttccagc

SEQ ID NO: 191 - DNA, 20 bp, Artificial Sequence
  Description of Artificial Sequence Synthetic oligonucleotide
acctggacggagtgtacgag

SEQ ID NO: 192 - DNA, 20 bp, Artificial Sequence
  Description of Artificial Sequence Synthetic oligonucleotide
tctcccaatagctggtcagg

SEQ ID NO: 193 - DNA, 18 bp, Artificial Sequence
  Description of Artificial Sequence Synthetic oligonucleotide
gctacgagctgcctgacg

SEQ ID NO: 194 - DNA, 18 bp, Artificial Sequence
  Description of Artificial Sequence Synthetic oligonucleotide
ggctggaagagtgcctca

SEQ ID NO: 195 - DNA, 45 bp, Artificial Sequence
  Description of Artificial Sequence Synthetic oligonucleotide
gtgtgttttgagactataagtatcccttggagaaccaccttgttg

SEQ ID NO: 196 - DNA, 52 bp, Artificial Sequence
  Description of Artificial Sequence Synthetic oligonucleotide
ccactttttcaagttgataacggactagccttatttaaacttgctatgctgt

SEQ ID NO: 197 - DNA, 50 bp, Artificial Sequence
  Description of Artificial Sequence Synthetic oligonucleotide
aatgatacggcgaccaccgagatctacacgcacaaaaggaaactcaccct

SEQ ID NO: 198 - DNA, 83 bp, Artificial Sequence
  Description of Artificial Sequence Synthetic oligonucleotide
  modified_base (25)..(30): a, c, t, g, unknown or other
caagcagaagacggcatacgagatnnnnnngtctcgtgggctcggagatgtgtataagagacaggccgcctaatggatcctag

SEQ ID NO: 199 - DNA, 109 bp, Artificial Sequence
  Description of Artificial Sequence Synthetic polynucleotide
caagcagaagacggcatacgagatcagcctcggtctcgtgggctcggagatgtgtataagagacaggtgttttgagactataagtatcccttggagaaccaccttgttg

SEQ ID NO: 200 - DNA, 29 bp, Artificial Sequence
  Description of Artificial Sequence Synthetic oligonucleotide
aatgatacggcgaccaccgagatctacac

SEQ ID NO: 201 - DNA, 87 bp, Artificial Sequence
  Description of Artificial Sequence Synthetic oligonucleotide
gtttaagagctaagctggaaacagcatagcaagtttaaataaggctagtccgttatcaactcgagagagtggcaccgagtcggtgct

SEQ ID NO: 202 - DNA, 87 bp, Artificial Sequence
  Description of Artificial Sequence Synthetic oligonucleotide
gtttaagagctaagctggaaacagcatagcaagtttaaataaggctagtccgttatcaactggaaacagtggcaccgagtcggtgct

SEQ ID NO: 203 - DNA, 87 bp, Artificial Sequence
  Description of Artificial Sequence Synthetic oligonucleotide
gtttaagagctaagctggaaacagcatagcaagtttaaataaggctagtccgttatcagctggaaacagcggcaccgagtcggtgct

SEQ ID NO: 204 - DNA, 87 bp, Artificial Sequence
  Description of Artificial Sequence Synthetic oligonucleotide
gtttaagagctaagctggaaacagcatagcaagtttaaataaggctagtccgttatcaactcgaaagagtggcaccgagtcggtgct

SEQ ID NO: 205 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaacgtgactccgagtcggagtt

SEQ ID NO: 206 - DNA, 87 bp, Artificial Sequence
  Description of Artificial Sequence Synthetic oligonucleotide
gtttaagagctaagctggaaacagcatagcaagtttaaataaggctagtccgttatcaactcgaaagagtgcaaccgagtcggttgt

SEQ ID NO: 207 - DNA, 87 bp, Artificial Sequence
  Description of Artificial Sequence Synthetic oligonucleotide
gtttaagagctaagctggaaacagcatagcaagtttaaataaggctagtccgttaccaacttgaacaagtggcaccgagtcggtgct

SEQ ID NO: 208 - DNA, 87 bp, Artificial Sequence
  Description of Artificial Sequence Synthetic oligonucleotide
gtttaagagctaagctggaaacagcatagcaagtttaaataaggctagtccgttatcaacgcgaaagcgtggcaccgagtcggtgct

SEQ ID NO: 209 - DNA, 87 bp, Artificial Sequence
  Description of Artificial Sequence Synthetic oligonucleotide
gtttaagagctaagctggaaacagcatagcaagtttaaataaggctagtccgttatcatcgagaaatcgaggcaccgagtcggtgct

SEQ ID NO: 210 - DNA, 87 bp, Artificial Sequence
  Description of Artificial Sequence Synthetic oligonucleotide
gtttaagagctaagctggaaacagcatagcaagtttaaataaggctagtccgttatcaactcgaaaaagtggcaccgagtcggtgct

SEQ ID NO: 211 - DNA, 87 bp, Artificial Sequence
  Description of Artificial Sequence Synthetic oligonucleotide
gtttaagagctaagctggaaacagcatagcaagtttaaataaggctagtccgttatcaactggagacagtggcaccgagtcggtgct

SEQ ID NO: 212 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtcaccgagtcggtgat

SEQ ID NO: 213 - DNA, 87 bp, Artificial Sequence
  Description of Artificial Sequence Synthetic oligonucleotide
gtttaagagctaagctggaaacagcatagcaagtttaaataaggctagtccgttatcaaggtgaaaacctggcaccgagtcggtgct

SEQ ID NO: 214 - DNA, 87 bp, Artificial Sequence
  Description of Artificial Sequence Synthetic oligonucleotide
gtttaagagctaagctggaaacagcatagcaagtttaaataaggctagtccgttatcaactagaaatagtggcaccgagtcggtgct

SEQ ID NO: 215 - DNA, 87 bp, Artificial Sequence
  Description of Artificial Sequence Synthetic oligonucleotide
gtttaagagctaagctggaaacagcatagcaagtttaaataaggctagtccgttatcaattcgaaagaatggcaccgagtcggtgct

SEQ ID NO: 216 - DNA, 87 bp, Artificial Sequence
  Description of Artificial Sequence Synthetic oligonucleotide
gtttaagagctaagctggaaacagcatagcaagtttaaataaggttaatccgttatcaacttgaaaaagtgacaccgagtcggtgtt

SEQ ID NO: 217 - DNA, 87 bp, Artificial Sequence
  Description of Artificial Sequence Synthetic oligonucleotide
gtttaagagctaagctggaaacagcatagcaagtttaaataaggctagtccgttatcaagctgaaaagctggcaccgagtcggtgct

SEQ ID NO: 218 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggcaccgagtcggtgct

SEQ ID NO: 219 - DNA, 87 bp, Artificial Sequence
  Description of Artificial Sequence Synthetic oligonucleotide
gtttaagagctaagctggaaacagcatagcaagtttaaataaggctagtccgttatcaacttgcaaaagtggcaccgagtcggtgct

SEQ ID NO: 220 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcatcgagtcgatgct

SEQ ID NO: 221 - DNA, 87 bp, Artificial Sequence
  Description of Artificial Sequence Synthetic oligonucleotide
gtttaagagctaagctggaaacagcatagcaagtttaaataaggctagtccgttatcaactcgaaagagtggtaccaagttggtact

SEQ ID NO: 222 - DNA, 87 bp, Artificial Sequence
  Description of Artificial Sequence Synthetic oligonucleotide
gtttaagagctaagctggatacagcatagcaagtttaaataaggctagtccgttatcaacttgaaaaagtggcaccgagtcggtgct

SEQ ID NO: 223 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaacgtggcaccgagtcggtgct

SEQ ID NO: 224 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcgccgagtcggcgct

SEQ ID NO: 225 - DNA, 87 bp, Artificial Sequence
  Description of Artificial Sequence Synthetic oligonucleotide
gtttaagagctaagctggaaacagcatagcaagtttaaataaggctagtccgttatcaacttgcgaaagtggcaccgagtcggtgct

SEQ ID NO: 226 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaggtgggacagagtctgtcct

SEQ ID NO: 227 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggaccgagtcggtcct

SEQ ID NO: 228 - DNA, 87 bp, Artificial Sequence
  Description of Artificial Sequence Synthetic oligonucleotide
gtttaagagctaagctggaaacagcatagcaagtttaaataaggttaatccgttatcagcgtgaaaacgcggcaccgagtcggtgct

SEQ ID NO: 229 - DNA, 87 bp, Artificial Sequence
  Description of Artificial Sequence Synthetic oligonucleotide
gtttaagtgctaagctggaaacagcatagcaagtttaaataaggctagtccgttatcaacttgaaaaagtggcaccgagtcggtgct

SEQ ID NO: 230 - DNA, 87 bp, Artificial Sequence
  Description of Artificial Sequence Synthetic oligonucleotide
gtttaagagctaagctggaaacagcatagcaagtttaaataaggttaatccgttatcagcttgaaaaagcggcaccgagtcggtgct

SEQ ID NO: 231 - DNA, 87 bp, Artificial Sequence
  Description of Artificial Sequence Synthetic oligonucleotide
gtttaagagctaagctggaaacagcatagcaagtttaaataaggctagtccgttatcacctggaaacaggggcaccgagtcggtgct

SEQ ID NO: 232 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctccgagtcggagct

SEQ ID NO: 233 - DNA, 87 bp, Artificial Sequence
  Description of Artificial Sequence Synthetic oligonucleotide
gtttaagagctaagctggaaacagcatagcaagtttaaataaggctagtccgttatcaacttgaaacagtggcaccgagtcggtgct

SEQ ID NO: 234 - DNA, 87 bp, Artificial Sequence
  Description of Artificial Sequence Synthetic oligonucleotide
gtttaagagctaagctggaaacagcatagcaagtttaaataaggctagtccgttaccaacttgaaaaagtggcaccgagtcggtgct

SEQ ID NO: 235 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggtaccgagtcggtact

SEQ ID NO: 236 - DNA, 87 bp, Artificial Sequence
  Description of Artificial Sequence Synthetic oligonucleotide
gtttaagagctaagctggaaacagcatagcaagtttaaataaggctagtccgttatcatcgtgaaaacgaggcaacgagtcgttgct

SEQ ID NO: 237 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaggtggcagcgagtcgctgct

SEQ ID NO: 238 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtgtcaccgagtcggtgat

SEQ ID NO: 239 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggaccaagttggtcct

SEQ ID NO: 240 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaggtggcaccgagtcggtgct

SEQ ID NO: 241 - DNA, 87 bp, Artificial Sequence
  Description of Artificial Sequence Synthetic oligonucleotide
gtttaagagctaagctggaaacagcatagcaagtttaaataaggctagtccgttatcaacatgaaaatgtggcaccgagtcggtgct

SEQ ID NO: 242 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcgccgagtcggcgct

SEQ ID NO: 243 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgcacccgagtcgggtgt

SEQ ID NO: 244 - DNA, 87 bp, Artificial Sequence
  Description of Artificial Sequence Synthetic oligonucleotide
gtttaagagctaagctggtaacagcatagcaagtttaaataaggctagtccgttatcaacttgaaaaagtggcaccgagtcggtgct

SEQ ID NO: 245 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggtccgagtcggacct

SEQ ID NO: 246 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcgccgagtcggtgct

SEQ ID NO: 247 - DNA, 87 bp, Artificial Sequence
  Description of Artificial Sequence Synthetic oligonucleotide
gtttaagagctaagctggaaacagcatagcaagtttaaataaggttaatccgttatcaacttgagaaagtggcaccgagtcggtgct

SEQ ID NO: 248 - DNA, 87 bp, Artificial Sequence
  Description of Artificial Sequence Synthetic oligonucleotide
gtttaagagctaagctggaaacagcatagcaagtttaaataaggctagtccgttaacaacttgaaaaagtggcaccgagtcggtgct

SEQ ID NO: 249 - DNA, 87 bp, Artificial Sequence
  Description of Artificial Sequence Synthetic oligonucleotide
gtttaagagctaagctggaaacagcatagcaagtttaaataaggctagtccgttatcaacctgagaaggtggcaccgagtcggtgct

SEQ ID NO: 250 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaacgtggaaccgagtcggttct

SEQ ID NO: 251 - DNA, 87 bp, Artificial Sequence
  Description of Artificial Sequence Synthetic oligonucleotide
gtttaagagctaagctggaaacagcatagcaagtttaaataaggctagtccgttatcaccttgaaaaagggacaccgagtcggtgtt

SEQ ID NO: 252 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcactgagtcggtgct

SEQ ID NO: 253 - DNA, 87 bp, Artificial Sequence
  Description of Artificial Sequence Synthetic oligonucleotide
gtttaagagctaagctggaaacagcatagcaagtttaaataaggttaatccgttatcaacttgaaaaagtggcaacgagtcgttgct

SEQ ID NO: 254 - DNA, 87 bp, Artificial Sequence
  Description of Artificial Sequence Synthetic oligonucleotide
gtttaagagctaagctggaaacagcatagcaagtttaaataaggctagtccgttatcactgtgaaaacagggcaccgagtcggtgct

SEQ ID NO: 255 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagtggcaccgagtcggtgct

SEQ ID NO: 256 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaacgtggcgacgagtcgtcgct

SEQ ID NO: 257 - DNA, 87 bp, Artificial Sequence
  Description of Artificial Sequence Synthetic oligonucleotide
gtttaagagctaagctggaaacagcctagcaagtttaaataaggctagtccgttatcaacttgaaaaagtggcaccgagtcggtgct

SEQ ID NO: 258 - DNA, 87 bp, Artificial Sequence
  Description of Artificial Sequence Synthetic oligonucleotide
gtttaagagctaagctggaaacagcaaagcaagtttaaataaggctagtccgttatcaacttgaaaaagtggcaccgagtcggtgct

SEQ ID NO: 259 - DNA, 87 bp, Artificial Sequence
  Description of Artificial Sequence Synthetic oligonucleotide
gtttaagagctaagctggaaacagcatagcacgtttaaataaggctagtccgttatcaacttgaaaaagtggcaccgagtcggtgct

SEQ ID NO: 260 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggtgccgagtcggcact

SEQ ID NO: 261 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgcgaccgagtcggtcgt

SEQ ID NO: 262 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggcccgagtcgggcct

SEQ ID NO: 263 - DNA, 87 bp, Artificial Sequence
  Description of Artificial Sequence Synthetic oligonucleotide
gtttaagagctaagctggaaacagcgtagcaagtttaaataaggctagtccgttatcaacttgaaaaagtggcaccgagtcggtgct

SEQ ID NO: 264 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtcgccgagtcggcgat

SEQ ID NO: 265 - DNA, 87 bp, Artificial Sequence
  Description of Artificial Sequence Synthetic oligonucleotide
gtttaagagctaagctggaaacagcatagcaagtttaaataaggctagtccgttatcaactagaaatagtggcgtcgagtcgacgct

SEQ ID NO: 266 - DNA, 87 bp, Artificial Sequence
  Description of Artificial Sequence Synthetic oligonucleotide
gtttaagagctaagctggaaacagcatagcaagtttaaataaggctagtccgttatcatctagaaatagaggcaccgagtcggtgct

SEQ ID NO: 267 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgacgccgagtcggcgtt

SEQ ID NO: 268 - DNA, 87 bp, Artificial Sequence
  Description of Artificial Sequence Synthetic oligonucleotide
gtttaagagctaagctggaaacagcatagcaagtttaaataaggctagtccgttatcaccttgaaaaaggggcaccgagtcggtgct

SEQ ID NO: 269 - DNA, 87 bp, Artificial Sequence
  Description of Artificial Sequence Synthetic oligonucleotide
gtttaagagctaagctggaaacagcatagcaagtttaaataaggctagtccgttatcaagatgaaaatctggcaccgagtcggtgct

SEQ ID NO: 270 - DNA, 87 bp, Artificial Sequence
  Description of Artificial Sequence Synthetic oligonucleotide
gtttaagagctaagctggaaacagcatagcatgtttaaataaggctagtccgttatcaacttgagaaagtggcaccgagtcggtgct

SEQ ID NO: 271 - DNA, 87 bp, Artificial Sequence
  Description of Artificial Sequence Synthetic oligonucleotide
gtttaagagctaagctggaaacagcatagcaagtttaaataaggctagtccgttatcagcttgagaaagcggcaccgagtcggtgct

SEQ ID NO: 272 - DNA, 87 bp, Artificial Sequence
  Description of Artificial Sequence Synthetic oligonucleotide
gtttaagagctaagctggaaacagcatagcaagtttaaataaggctagtccgttctcaacttgaaaaagtggcaccgagtcggtgct

SEQ ID NO: 273 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggctccgagtcggagct

SEQ ID NO: 274 - DNA, 87 bp, Artificial Sequence
  Description of Artificial Sequence Synthetic oligonucleotide
gtttaagagctaagctggaaacagcatagctagtttaaataaggctagtccgttatcaacttgagaaagtggcaccgagtcggtgct

SEQ ID NO: 275 - DNA, 87 bp, Artificial Sequence
  Description of Artificial Sequence Synthetic oligonucleotide
gtttaagagctaagctggaaacagcatagcaagtttaaataaggctagtccgttatcaacttgaacaagtggcaccgagtcggtgct

SEQ ID NO: 276 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcaacgagtcgttgct

SEQ ID NO: 277 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggtaccaagttggtact

SEQ ID NO: 278 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagcggcaccgagtcggtgct

SEQ ID NO: 279 - DNA, 87 bp, Artificial Sequence
  Description of Artificial Sequence Synthetic oligonucleotide
gtttaagagctaagctggaaacagcatagcaagtttaaataaggctagtccgttatcaacttgtaaaagtggcaccgagtcggtgct

SEQ ID NO: 280 - DNA, 87 bp, Artificial Sequence
  Description of Artificial Sequence Synthetic oligonucleotide
gtttaaccgctaagctggaaacagcatagcaagtttaaataaggctagtccgttatcaacttgaaaaagtggcaccgagtcggtgct

SEQ ID NO: 281 - DNA, 87 bp, Artificial Sequence
  Description of Artificial Sequence Synthetic oligonucleotide
gtttaagagctaagctggaaacagcatagcaagtttaaataaggctagtccgttatcaattggaaacaatggcaccgagtcggtgct

SEQ ID NO: 282 - DNA, 87 bp, Artificial Sequence
  Description of Artificial Sequence Synthetic oligonucleotide
gtttaagagctaagctggaaacagcatagcaagtttaaataaggctagtccgttatcaccttgaaaaagtggcaccgagtcggtgct

SEQ ID NO: 283 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgacaccgagtcggtgtt

SEQ ID NO: 284 - DNA, 87 bp, Artificial Sequence
  Description of Artificial Sequence Synthetic oligonucleotide
gtttaagagctaagctggaaacagcatagcaagtttaaataaggctagtccgttatcaacaagaaattgtggcaccgagtcggtgct

SEQ ID NO: 285 - DNA, 87 bp, Artificial Sequence
  Description of Artificial Sequence Synthetic oligonucleotide
gtttaacggctaagctggaaacagcatagcaagtttaaataaggctagtccgttatcaacttgaaaaagtggcaccgagtcggtgct

SEQ ID NO: 286 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggtcccgagtcgggact

SEQ ID NO: 287 - DNA, 87 bp, Artificial Sequence
  Description of Artificial Sequence Synthetic oligonucleotide
gtttaagagctaagctggaaacagcatagcaagtttaaataaggctagtccgttatcaacgtgagaacgtggcaccgagtcggtgct

SEQ ID NO: 288 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggaaccgagtcggttct

SEQ ID NO: 289 - DNA, 87 bp, Artificial Sequence
  Description of Artificial Sequence Synthetic oligonucleotide
gtttaagagctaagctggaaacagcatagcaggtttaaataaggctagtccgttatcaacttgaaaaagtggcaccgagtcggtgct

SEQ ID NO: 290 - DNA, 87 bp, Artificial Sequence
  Description of Artificial Sequence Synthetic oligonucleotide
gtttaagagctaagctggaaacagcatagcaagtttaaataaggctagtccgttatcaacatgagaatgtggcaccgagtcggtgct

SEQ ID NO: 291 - DNA, 87 bp, Artificial Sequence
  Description of Artificial Sequence Synthetic oligonucleotide
gtttaagagctaagctggaaacagcatagcaagtttaaataaggctagtccgttatcaacttcaaaaagtggcaccgagtcggtgct

SEQ ID NO: 292 - DNA, 87 bp, Artificial Sequence
  Description of Artificial Sequence Synthetic oligonucleotide
gtttaagagctaagctgtaaacagcatagcaagtttaaataaggctagtccgttatcaacttgaaaaagtggcaccgagtcggtgct

SEQ ID NO: 293 - DNA, 87 bp, Artificial Sequence
  Description of Artificial Sequence Synthetic oligonucleotide
gtttaagagctaagctggaaacagcatagcaagtttaaataaggctagtccgttatcaacttcataaagtggcaccgagtcggtgct

SEQ ID NO: 294 - DNA, 87 bp, Artificial Sequence
  Description of Artificial Sequence Synthetic oligonucleotide
gtttaagagctaagctggaaacagcatagcaagtttaaataaggctagtccgtgatcaacttgaaaaagtggcaccgagtcggtgct

SEQ ID NO: 295 - DNA, 87 bp, Artificial Sequence
  Description of Artificial Sequence Synthetic oligonucleotide
gtttaagagctaagctggaaacagcatagcaagtttaaataaggctagtccgttatcacctagaaatagggtcaccgagtcggtgat

SEQ ID NO: 296 - DNA, 87 bp, Artificial Sequence
  Description of Artificial Sequence Synthetic oligonucleotide
gtttaagagctaagctggaaacagcatagcaagtttaaataaggctagtccgttatcaagatgaaaatctgacaccgagtcggtgtt

SEQ ID NO: 297 - DNA, 87 bp, Artificial Sequence
  Description of Artificial Sequence Synthetic oligonucleotide
gtttaagagctaagctggaaacggcatagcaagtttaaataaggctagtccgttatcaacttgaaaaagtggcaccgagtcggtgct

SEQ ID NO: 298 - DNA, 87 bp, Artificial Sequence
  Description of Artificial Sequence Synthetic oligonucleotide
gtttaagagctaagctggaaacagcatagcaagtttaaataaggctagtccgttatcaagttgaaaaactggcaccgagtcggtgct

SEQ ID NO: 299 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtcaccgagtcggtgct

SEQ ID NO: 300 - DNA, 87 bp, Artificial Sequence
  Description of Artificial Sequence Synthetic oligonucleotide
gtttaagagctaagctggagacagcatagcaagtttaaataaggctagtccgttatcaacttgaaaaagtggcaccgagtcggtgct

SEQ ID NO: 301 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggacccgagtcgggtct

SEQ ID NO: 302 - DNA, 87 bp, Artificial Sequence
  Description of Artificial Sequence Synthetic oligonucleotide
gtttaagagctaagctggaaacagcatagcaagtttaaataaggctagtccgttatcaagtggaaacactggcaccaagttggtgct

SEQ ID NO: 303 - DNA, 87 bp, Artificial Sequence
  Description of Artificial Sequence Synthetic oligonucleotide
gtttaagagctaagctggaaacagcatagcaagtttaaataaggcttgtccgttaccaacttgaaaaagtggcaccgattcggtgct

SEQ ID NO: 304 - DNA, 87 bp, Artificial Sequence
  Description of Artificial Sequence Synthetic oligonucleotide
gtttaagagctaagctggaaacagcatagcaagtttaaataaggctagtccgttatcaacttgacaaagtggcaccgagtcggtgct

SEQ ID NO: 305 - DNA, 87 bp, Artificial Sequence
  Description of Artificial Sequence Synthetic oligonucleotide
gtttaagagctaagctggaaacagcatagcaagtttaaataaggctagtccgttatcaatctgaaaagatggcaccgagtcggtgct

SEQ ID NO: 306 - DNA, 87 bp, Artificial Sequence
  Description of Artificial Sequence Synthetic oligonucleotide
gtttaagagctaagctggaaacagcatagcaagtttaaataaggctagtccgttatcaagttgagaaactggcaccgagtcggtgct

SEQ ID NO: 307 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcagcgagtcgctgct

SEQ ID NO: 308 - DNA, 87 bp, Artificial Sequence
  Description of Artificial Sequence Synthetic oligonucleotide
gtttaagagctaagctggaaccagcatagcaagtttaaataaggctagtccgttatcaacttgaaaaagtggcaccgagtcggtgct

SEQ ID NO: 309 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggaaacgagtcgtttct

SEQ ID NO: 310 - DNA, 87 bp, Artificial Sequence
  Description of Artificial Sequence Synthetic oligonucleotide
gtttaagagctaagctggaaacagcatagcaagtttaaataaggcttgtccgttatcaacttgaaaaagtggcaccgagtcggtgct

SEQ ID NO: 311 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggaagcgagtcgcttct

SEQ ID NO: 312 - DNA, 87 bp, Artificial Sequence
  Description of Artificial Sequence Synthetic oligonucleotide
gtttaagagctaagctggaaacagcatagcaagtttaaataaggctagtccgttatcaacttgaaagagtggcaccgagtcggtgct

SEQ ID NO: 313 - DNA, 87 bp, Artificial Sequence
  Description of Artificial Sequence Synthetic oligonucleotide
gtttaagagctaagctggaaacagcatagcaagtttaaataaggctagtccgttatcaactttacaaagtggcaccgagtcggtgct

SEQ ID NO: 314 - DNA, 87 bp, Artificial Sequence
  Description of Artificial Sequence Synthetic oligonucleotide
gtttaatagctaagctggaaacagcatagcaagtttaaataaggctagtccgttatcaacttgaaaaagtggcaccgagtcggtgct

SEQ ID NO: 315 - DNA, 87 bp, Artificial Sequence
  Description of Artificial Sequence Synthetic oligonucleotide
gtttaagagctaagctggaaacagcatagcaggtttaaataaggctagtccgttatcaacttgacaaagtggcaccgagtcggtgct

SEQ ID NO: 316 - DNA, 87 bp, Artificial Sequence
  Description of Artificial Sequence Synthetic oligonucleotide
gtttaacagctaagctggaaacagcatagcaagtttaaataaggctagtccgttatcaacttgaaaaagtggcaccgagtcggtgct

SEQ ID NO: 317 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtgacaccgagtcggtgtt

SEQ ID NO: 318 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggggcgagtcgcccct

SEQ ID NO: 319 - DNA, 87 bp, Artificial Sequence
  Description of Artificial Sequence Synthetic oligonucleotide
gtttaagagctaagctggaaacagcatagcaagtttaaataaggctagtccgttatcaacttgagcaagtggcaccgagtcggtgct

SEQ ID NO: 320 - DNA, 87 bp, Artificial Sequence
  Description of Artificial Sequence Synthetic oligonucleotide
gtttaagagctaagctggcaacagcatagcaagtttaaataaggctagtccgttatcaacttgaaaaagtggcaccgagtcggtgct

SEQ ID NO: 321 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgacaccgagtcggtgct

SEQ ID NO: 322 - DNA, 87 bp, Artificial Sequence
  Description of Artificial Sequence Synthetic oligonucleotide
gtttaagagctaagctggaaacagcatagcaagtttaaataaggctagtccgttatcaccttgaaaaaggggcaccaagttggtgct

SEQ ID NO: 323 - DNA, 87 bp, Artificial Sequence
  Description of Artificial Sequence Synthetic oligonucleotide
gtttaagagctaagctggaaacagcatagcaagtttaaataaggttaatccgttatcaacttgaaaaagtggcaccgagtcggtgct

SEQ ID NO: 324 - DNA, 87 bp, Artificial Sequence
  Description of Artificial Sequence Synthetic oligonucleotide
gtttaagagctaagctggaaacagcatagcaagtttaaataaggctagtccgttatcaacttaataaagtggcaccgagtcggtgct

SEQ ID NO: 325 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggcactgagtcagtgct

SEQ ID NO: 326 - DNA, 87 bp, Artificial Sequence
  Description of Artificial Sequence Synthetic oligonucleotide
gtttaaaagctaagctggaaacagcatagcaagtttaaataaggctagtccgttatcaacttgaaaaagtggcaccgagtcggtgct

SEQ ID NO: 327 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgactcggtgct

SEQ ID NO: 328 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcgataccgagtcggtatt

SEQ ID NO: 329 - DNA, 87 bp, Artificial Sequence
  Description of Artificial Sequence Synthetic oligonucleotide
gtttaagagctaagctggaaacagcatagcaagtttaaataaggctagtccgttatcaacttgaaatagtggcaccgagtcggtgct

SEQ ID NO: 330 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaacgtggtcccgagtcgggact

SEQ ID NO: 331 - DNA, 87 bp, Artificial Sequence
  Description of Artificial Sequence Synthetic oligonucleotide
gtttaagagctaagctggaaacagcatagcaagtttaaataaggctagtccgttttcaacttgaaaaagtggcaccgagtcggtgct

SEQ ID NO: 332 - DNA, 87 bp, Artificial Sequence
  Description of Artificial Sequence Synthetic oligonucleotide
gtttaagagctaagctggaaacagcatagcaagtttaaataaggctagtccgttatcaaatggaaacattgacaccgagtcggtgtt

SEQ ID NO: 333 - DNA, 87 bp, Artificial Sequence
  Description of Artificial Sequence Synthetic oligonucleotide
gtttaagagctaagctggaaacagcatagcaagtttaaataaggctagtccgtcatcaacttgaaaaagtggcaccgagtcggtgct

SEQ ID NO: 334 - DNA, 87 bp, Artificial Sequence
  Description of Artificial Sequence Synthetic oligonucleotide
gtttaagagctaagctggaaacagcatagcaagtttaaataaggctagtccgttatcaacttggaaaagtggcaccgagtcggtgct

SEQ ID NO: 335 - DNA, 87 bp, Artificial Sequence
  Description of Artificial Sequence Synthetic oligonucleotide
gtttaagagctaagctggaaacagcatagcaagtttaaataaggctagtccgttatcatcatgaaaatgaggcaccgagtcggtgct

SEQ ID NO: 336 - DNA, 87 bp, Artificial Sequence
  Description of Artificial Sequence Synthetic oligonucleotide
gtttaagagctaagctggaaacagcatagcacgtttaaataaggctagtccgttatcaacttgagaaagtggcaccgagtcggtgct

SEQ ID NO: 337 - DNA, 87 bp, Artificial Sequence
  Description of Artificial Sequence Synthetic oligonucleotide
gtttaagagctaagctggaaacagcatagcaagtttaaataaggctagtccgctatcaacttgtaaaagtggcaccgagtcggtgct

SEQ ID NO: 338 - DNA, 87 bp, Artificial Sequence
  Description of Artificial Sequence Synthetic oligonucleotide
gtttaagagctaagctggaaacagcatagcaagtttaaataaggctagtccgctatcaacttgaaaaagtggcaccgagtcggtgct

SEQ ID NO: 339 - DNA, 87 bp, Artificial Sequence
  Description of Artificial Sequence Synthetic oligonucleotide
gtttaagagctaagctggaaacagcatagcaagtttaaataaggctagtccgttatcaacttgcataagtggcaccgagtcggtgct

SEQ ID NO: 340 - DNA, 87 bp, Artificial Sequence
  Description of Artificial Sequence Synthetic oligonucleotide
gtttaagagctaagctggaaacagcatagcaagtttaaataaggctagtccgttatcaacttgaataagtggcaccgagtcggtgct

SEQ ID NO: 341 - DNA, 87 bp, Artificial Sequence
  Description of Artificial Sequence Synthetic oligonucleotide
gtttaagagctaagctggaaacagcatagcaagtttaaataaggctagtccgttagcaacttgaaaaagtggcaccgagtcggtgct

SEQ ID NO: 342 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggtaccgagtcggtact

SEQ ID NO: 343 - DNA, 87 bp, Artificial Sequence
  Description of Artificial Sequence Synthetic oligonucleotide
gtttaagagctaagctggaaacagcagagcaagtttaaataaggctagtccgttatcaacttgaaaaagtggcaccgagtcggtgct

SEQ ID NO: 344 - DNA, 87 bp, Artificial Sequence
  Description of Artificial Sequence Synthetic oligonucleotide
gtttaatcgctaagctggaaacagcatagcaagtttaaataaggctagtccgttatcaacttgaaaaagtggcaccgagtcggtgct

SEQ ID NO: 345 - DNA, 87 bp, Artificial Sequence
  Description of Artificial Sequence Synthetic oligonucleotide
gtttaagagctaagctggaaacagcatagcatgtttaaataaggctagtccgttatcaacttgaaaaagtggcaccgagtcggtgct

SEQ ID NO: 346 - DNA, 87 bp, Artificial Sequence
  Description of Artificial Sequence Synthetic oligonucleotide
gtttaagagctaagctggaaacagcatagcgagtttaaataaggctagtccgttatcaacttgaaaaagtggcaccgagtcggtgct

SEQ ID NO: 347 - DNA, 87 bp, Artificial Sequence
  Description of Artificial Sequence Synthetic oligonucleotide
gtttaagagctaagctggaaacagcatagcaagtttaaataaggctagtccgttatcaacttgcagaagtggcaccgagtcggtgct

SEQ ID NO: 348 - DNA, 87 bp, Artificial Sequence
  Description of Artificial Sequence Synthetic oligonucleotide
gtttaagagctaagctggaagcagcatagcaagtttaaataaggctagtccgttatcaacttgaaaaagtggcaccgagtcggtgct

SEQ ID NO: 349 - DNA, 87 bp, Artificial Sequence
  Description of Artificial Sequence Synthetic oligonucleotide
gtttaagagctaagctggaaacagcatagcaagtttaaataaggctagtccgttatcatgttgaaaaacaggcaccgagtcggtgct

SEQ ID NO: 350 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtgccaccgagtcggtggt

SEQ ID NO: 351 - DNA, 87 bp, Artificial Sequence
  Description of Artificial Sequence Synthetic oligonucleotide
gtttaagagctaagctggaaacagcatagcaagtttaaataaggctagtccgttatcaagttgaaaaactggcctcgagtcgaggct

SEQ ID NO: 352 - DNA, 87 bp, Artificial Sequence
  Description of Artificial Sequence Synthetic oligonucleotide
gtttaagagctaagctggaaacagcatagcaagtttaaataaggctagtccgttatcatgtagaaatacaggcaccgagtcggtgct

SEQ ID NO: 353 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggtaccgagtcggtact

SEQ ID NO: 354 - DNA, 87 bp, Artificial Sequence
  Description of Artificial Sequence Synthetic oligonucleotide
gtttaagagctaagctggaaacagcatagcaagtttaaataaggctagtccgtaatcaacttgaaaaagtggcaccgagtcggtgct

SEQ ID NO: 355 - DNA, 87 bp, Artificial Sequence
  Description of Artificial Sequence Synthetic oligonucleotide
gtttaagagctaagctggaaacagcacagcaagtttaaataaggctagtccgttatcaacttgaaaaagtggcaccgagtcggtgct

SEQ ID NO: 356 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaacgagtcgttgct

SEQ ID NO: 357 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcatcgagtcggtgct

SEQ ID NO: 358 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggccccgagtcggggct

SEQ ID NO: 359 - DNA, 87 bp, Artificial Sequence
  Description of Artificial Sequence Synthetic oligonucleotide
gtttaagagctaagctggaaacagcatagcaagtttaaataaggctagtccgttatcaacttgacaaagtggcaccgaatcggtgct

SEQ ID NO: 360 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggaaccgagtcggttct

SEQ ID NO: 361 - DNA, 87 bp, Artificial Sequence
  Description of Artificial Sequence Synthetic oligonucleotide
gtttaagagctaagctggaaacagcatagcaagtttaaataaggctagtccgttatcaattggaaacaatggcatcgagtcgatgct

SEQ ID NO: 362 - DNA, 87 bp, Artificial Sequence
  Description of Artificial Sequence Synthetic oligonucleotide
gtttaagagctaagctggaaacagcatagcaagtttaaataaggctagtccgttatcaacttgaagaagtggcaccgagtcggtgct

SEQ ID NO: 363 - DNA, 87 bp, Artificial Sequence
  Description of Artificial Sequence Synthetic oligonucleotide
gtttaagagctaagctggaaacagcatagcaagtttaaataaggctagtccgttatcaacttgaagaagtggcaccgattcggtgct

SEQ ID NO: 364 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggcacagagtctgtgct

SEQ ID NO: 365 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggcaccaagttggtgct

SEQ ID NO: 366 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgaccccgagtcggggtt

SEQ ID NO: 367 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggccccgagtcggggct

SEQ ID NO: 368 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggtgct

SEQ ID NO: 369 - DNA, 87 bp, Artificial Sequence
  Description of Artificial Sequence Synthetic oligonucleotide
gtttaagagctaagctggaaacagcatagcatgtttaaataaggctagtccgttatcaacttgataaagtggcaccgagtcggtgct

SEQ ID NO: 370 - DNA, 87 bp, Artificial Sequence
  Description of Artificial Sequence Synthetic oligonucleotide
gtttaagagctaagctgggaacagcatagcaagtttaaataaggctagtccgttatcaacttgaaaaagtggcaccgagtcggtgct

SEQ ID NO: 371 - DNA, 87 bp, Artificial Sequence
  Description of Artificial Sequence Synthetic oligonucleotide
gtttaagagctaagctggaaacagcatagcaagtttaaataaggcttgtccgttatcaacttgtaaaagtggcaccgagtcggtgct

SEQ ID NO: 372 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtgggaccgagtcggtcct

SEQ ID NO: 373 - DNA, 87 bp, Artificial Sequence
  Description of Artificial Sequence Synthetic oligonucleotide
gtttaagagctaagctggaaacagcatagcaagtttaaataaggctagtccgttatcaacttgcaaaagtggcaccgattcggtgct

SEQ ID NO: 374 - DNA, 87 bp, Artificial Sequence
  Description of Artificial Sequence Synthetic oligonucleotide
gtttaagagctaagctggaaacagcatagcaagtttaaataaggcaagtccgttatcaacttgaaaaagtggcaccgagtcggtgct

SEQ ID NO: 375 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgcaaccgagtcggttgt

SEQ ID NO: 376 - DNA, 87 bp, Artificial Sequence
  Description of Artificial Sequence Synthetic oligonucleotide
gtttaagagctaagctggaaacagcatagcaagtttaaataaggctagtccgttatcaactagagatagtggcaccgagtcggtgct

SEQ ID NO: 377 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgaaaccgagtcggtttt

SEQ ID NO: 378 - DNA, 87 bp, Artificial Sequence
  Description of Artificial Sequence Synthetic oligonucleotide
gtttaagagctaagctggacacagcatagcaagtttaaataaggctagtccgttatcaacttgaaaaagtggcaccgagtcggtgct

SEQ ID NO: 379 - DNA, 87 bp, Artificial Sequence
  Description of Artificial Sequence Synthetic oligonucleotide
gtttaagagctaagctggaaacagcatagcaagtttaaataaggttaatccgttatcaacttgaaaaagtggagccgagtcggctct

SEQ ID NO: 380 - DNA, 87 bp, Artificial Sequence
  Description of Artificial Sequence Synthetic oligonucleotide
gtttaagagctaagctggaaacagcataacaagtttaaataaggctagtccgtaatcaacttgtaaaagtggcaccgagtcggtgct

SEQ ID NO: 381 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgccaccgagtcggtggt

SEQ ID NO: 382 - DNA, 87 bp, Artificial Sequence
  Description of Artificial Sequence Synthetic oligonucleotide
gtttaagagctaagctggaaacagcatagtaagtttaaataaggctagtccgttatcaacttgaaaaagtggcaccgagtcggtgct

SEQ ID NO: 383 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggtgtt

SEQ ID NO: 384 - DNA, 87 bp, Artificial Sequence
  Description of Artificial Sequence Synthetic oligonucleotide
gtttaagagctaagctggaaacagcatagcaagtttaaataaggttaatccgttatcatcttgaaaaagagggaccgagtcggtcct

SEQ ID NO: 385 - DNA, 87 bp, Artificial Sequence
  Description of Artificial Sequence Synthetic oligonucleotide
gtttaagagctaagctggaaacagcatagcaagtttaaataaggctagtccgttatcaacttgataaagtggcaccgagtcggtgct

SEQ ID NO: 386 - DNA, 87 bp, Artificial Sequence
  Description of Artificial Sequence Synthetic oligonucleotide
gtttaagagctaagctggaaacagcatagcaagtttaaataaggctagtccgttatcaacttggtaaagtggcaccgagtcggtgct

SEQ ID NO: 387 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcaccgagtcggtgct

SEQ ID NO: 388 - DNA, 87 bp, Artificial Sequence
  Description of Artificial Sequence Synthetic oligonucleotide
gtttaagggctaagctggaaacagcatagcaagtttaaataaggctagtccgttatcaacttgaaaaagtggcaccgagtcggtgct

SEQ ID NO: 389 - DNA, 87 bp, Artificial Sequence
  Description of Artificial Sequence Synthetic oligonucleotide
gtttaagagctaagctggaaacagcatagcaagtttaaataaggcaagtccgttatcaacttgacaaagtggcaccgagtcggtgct

SEQ ID NO: 390 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacagagtctgtgct

SEQ ID NO: 391 - DNA, 87 bp, Artificial Sequence
  Description of Artificial Sequence Synthetic oligonucleotide
gtttaagagctaagctggaaacagcatagccagtttaaataaggctagtccgttatcaacttgaaaaagtggcaccgagtcggtgct

SEQ ID NO: 392 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaaggggcaccgagtcggtgct

SEQ ID NO: 393 - DNA, 87 bp, Artificial Sequence
  Description of Artificial Sequence Synthetic oligonucleotide
gtttaagagctaagctggaaacagcatggcaagtttaaataaggctagtccgttatcaacttgaaaaagtggcaccgagtcggtgct

SEQ ID NO: 394 - DNA, 87 bp, Artificial Sequence
  Description of Artificial Sequence Synthetic oligonucleotide
gtttaagagctaagctggaaacagcatagcatgtttaaataaggctagtccgttatcaacttaaaaaagtggcaccgaatcggtgct

SEQ ID NO: 395 - DNA, 87 bp, Artificial Sequence
  Description of Artificial Sequence Synthetic oligonucleotide
gtttaagagctaagctggaaacagcatagcaagtttaaataaggctagtccgttatcaactagaaaaagtggcaccgagtcggtgct

SEQ ID NO: 396 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgaatcggtgct

SEQ ID NO: 397 - DNA, 87 bp, Artificial Sequence
  Description of Artificial Sequence Synthetic oligonucleotide
gtttaagagctaagctggaaacagcatagcaagtttaaataaggctagtccgttatcaacttaaaaaagtggcaccgagtcggtgct

SEQ ID NO: 398 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaggtggcaccgagtcggtgct

SEQ ID NO: 399 - DNA, 87 bp, Artificial Sequence
  Description of Artificial Sequence Synthetic oligonucleotide
gtttaagagctaagctggaaacagcatagcaagtttaaataaggctagaccgttatcaacttgaaaaagtggcaccgagtcggtgct

SEQ ID NO: 400 - DNA, 87 bp, Artificial Sequence
  Description of Artificial Sequence Synthetic oligonucleotide
gtttaagagctaagctggaaacagcatagcaagtttaaataaggctagtccgttatcaactggaaaaagtggcaccgagtcggtgct

SEQ ID NO: 401 - DNA, 87 bp, Artificial Sequence
  Description of Artificial Sequence Synthetic oligonucleotide
gtttaagagctaagctggaaacagcatagcgagtttaaataaggctagtccgttatcaacttgataaagtggcaccgagtcggtgct

SEQ ID NO: 402 - DNA, 87 bp, Artificial Sequence
  Description of Artificial Sequence Synthetic oligonucleotide
gtttaagagctaagctggaaacagcatagcaagtttaaataaggccagtccgttctcaacttgacaaagtggcaccgagtcggtgct

SEQ ID NO: 403 - DNA, 87 bp, Artificial Sequence
  Description of Artificial Sequence Synthetic oligonucleotide
gtttaagagctaagctggaaacagcatagcaagtttaaataaggctagtccgttatcacattgaaaaatggtcaccgagtcggtgat

SEQ ID NO: 404 - DNA, 87 bp, Artificial Sequence
  Description of Artificial Sequence Synthetic oligonucleotide
gtttaagagctaagctggaaacagcatagcaagtttaaataaggctagtccgttatcacattgaaaaatgggcaccgagtcggtgct

SEQ ID NO: 405 - DNA, 87 bp, Artificial Sequence
  Description of Artificial Sequence Synthetic oligonucleotide
gtttaagagctaagctggaaacagcatagcaagtttaaataaggctagtccgtgatcaacttgtaaaagtggcaccgactcggtgct

SEQ ID NO: 406 - DNA, 87 bp, Artificial Sequence
  Description of Artificial Sequence Synthetic oligonucleotide
gtttaagagctaagctggaaacagcatagcaagtttaaataaggctagtccgttatcatcctgaaaaggaggcactgagtcagtgct

SEQ ID NO: 407 - DNA, 87 bp, Artificial Sequence
  Description of Artificial Sequence Synthetic oligonucleotide
gtttaagagctaagctggaaacagcatagcaagtttaaataaggctagtccgttatcaacttgttaaagtggcaccgagtcggtgct

SEQ ID NO: 408 - DNA, 87 bp, Artificial Sequence
  Description of Artificial Sequence Synthetic oligonucleotide
gtttaagagctaagctggaatcagcatagcaagtttaaataaggctagtccgttatcaacttgaaaaagtggcaccgagtcggtgct

SEQ ID NO: 409 - DNA, 87 bp, Artificial Sequence
  Description of Artificial Sequence Synthetic oligonucleotide
gtttaagagctaagctggaaacagcatagcaagtttaaataaggctagtccgttatcatcttgaaaaagaggcaccgagtcggtgct

SEQ ID NO: 410 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcagcgagtcgctgct

SEQ ID NO: 411 - DNA, 87 bp, Artificial Sequence
  Description of Artificial Sequence Synthetic oligonucleotide
gtttaagagctaagctggaaacagcatagcaagtttaaataaggctagtccgttatcaactttagaaagtggcaccgagtcggtgct

SEQ ID NO: 412 - DNA, 87 bp, Artificial Sequence
  Description of Artificial Sequence Synthetic oligonucleotide
gtttaagagctaagctggaaacagcatagcaagtttaaataaggctagtccgttatcatcttgagaaagaggcaccgagtcggtgct

SEQ ID NO: 413 - DNA, 87 bp, Artificial Sequence
  Description of Artificial Sequence Synthetic oligonucleotide
gtttaagagctaagctggaaacagcatagcaagtttaaataaggctagtccgttatcaccttgagaaaggggcaccgagtcggtgct

SEQ ID NO: 414 - DNA, 87 bp, Artificial Sequence
  Description of Artificial Sequence Synthetic oligonucleotide
gtttaagagctaagctggaaacagaatagcaagtttaaataaggctagtccgttatcaacttgaaaaagtggcaccgagtcggtgct

SEQ ID NO: 415 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcttcgagtcgaagct

SEQ ID NO: 416 - DNA, 87 bp, Artificial Sequence
  Description of Artificial Sequence Synthetic oligonucleotide
gtttaagagctaagctggaaacagcatagcaagtttaaataaggctagtccgttatcaactttaaaaagtggcaccgagtcggtgct

SEQ ID NO: 417 - DNA, 87 bp, Artificial Sequence
  Description of Artificial Sequence Synthetic oligonucleotide
gtttaagagctaagctggaaacagtatagcaagtttaaataaggctagtccgttatcaacttgaaaaagtggcaccgagtcggtgct

SEQ ID NO: 418 - DNA, 87 bp, Artificial Sequence
  Description of Artificial Sequence Synthetic oligonucleotide
gtttaagagctaagctggaaacagcatagcaagtttaaataaggctagtccgttgtcaacttgaaaaagtggcaccgagtcggtgct

SEQ ID NO: 419 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggccacgagtcgtggct

SEQ ID NO: 420 - DNA, 87 bp, Artificial Sequence
  Description of Artificial Sequence Synthetic oligonucleotide
gtttaagcgctaagctggaaacagcatagcaagtttaaataaggctagtccgttatcaacttgaaaaagtggcaccgagtcggtgct

SEQ ID NO: 421 - DNA, 87 bp, Artificial Sequence
  Description of Artificial Sequence Synthetic oligonucleotide
gtttaagagctaagctggaaacagcatagcaagtttaaataaggctagtccgttatcaactcgaaagagtgataccgagtcggtatt

SEQ ID NO: 422 - DNA, 87 bp, Artificial Sequence
  Description of Artificial Sequence Synthetic oligonucleotide
gtttaagagctaagctggaaacagcatagcaagtttaaataaggctagtccgttatcatcttgaaaaagaggaaccgagtcggttct

SEQ ID NO: 423 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaggtggtatcgagtcgatact

SEQ ID NO: 424 - DNA, 87 bp, Artificial Sequence
  Description of Artificial Sequence Synthetic oligonucleotide
gtttaagagctaagctggaaacagcatagcaagtttaaataaggctagtccgttatcaacttataaaagtggcaccgactcggtgct

SEQ ID NO: 425 - DNA, 87 bp, Artificial Sequence
  Description of Artificial Sequence Synthetic oligonucleotide
gtttaagagctaagctggaaacagcatcgcaagtttaaataaggctagtccgttatcaacttgaaaaagtggcaccgagtcggtgct

SEQ ID NO: 426 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccaagttggtgct

SEQ ID NO: 427 - DNA, 87 bp, Artificial Sequence
  Description of Artificial Sequence Synthetic oligonucleotide
gtttaagagctaagctggaaacagcatagctagtttaaataaggctagtccgttatcaacttgaaaaagtggcaccgagtcggtgct

SEQ ID NO: 428 - DNA, 87 bp, Artificial Sequence
  Description of Artificial Sequence Synthetic oligonucleotide
gtttaagagctaagctggaaacagcatagcaagtttaaataaggttaatccgttatcatcttgaaaaagagacaccgagtcggtgtt

SEQ ID NO: 429 - DNA, 87 bp, Artificial Sequence
  Description of Artificial Sequence Synthetic oligonucleotide
gtttaagagctaagctggaaacagcatagcaagtttaaataaggctagtccgttatcaacttgggaaagtggcaccgagtcggtgct

SEQ ID NO: 430 - DNA, 87 bp, Artificial Sequence
  Description of Artificial Sequence Synthetic oligonucleotide
gtttaagagctaagctggaaacagcattgcaagtttaaataaggctagtccgttatcaacttgaaaaagtggcaccgagtcggtgct

SEQ ID NO: 431 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctgcgagtcgcagct

SEQ ID NO: 432 - DNA, 87 bp, Artificial Sequence
  Description of Artificial Sequence Synthetic oligonucleotide
gtttaagagctaagctggaaacagcatagcaagtttaaataaggctagtccgttatcaacttgataaagtggcaccgactcggtgct

SEQ ID NO: 433 - DNA, 87 bp, Artificial Sequence
  Description of Artificial Sequence Synthetic oligonucleotide
gtttaagagctaagctggaaacagcatagcaagtttaaataaggctagtccgttatcaacttgaacaagtggcaccgactcggtgct

SEQ ID NO: 434 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgattcggtgct

SEQ ID NO: 435 - DNA, 87 bp, Artificial Sequence
  Description of Artificial Sequence Synthetic oligonucleotide
gtttaagagctaagctggaaacagcatagcaagtttaaataaggctagtccgttatcaatttgaaaaaatggaaccgagtcggttct

SEQ ID NO: 436 - DNA, 87 bp, Artificial Sequence
  Description of Artificial Sequence Synthetic oligonucleotide
gtttaagagctaagctggaaacagcatagcaagtttaaataaggcttgtccgttatcaacttgaaaaagtggcaccgtgtcggtgct

SEQ ID NO: 437 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgtgtcggtgct

SEQ ID NO: 438 - DNA, 87 bp, Artificial Sequence
  Description of Artificial Sequence Synthetic oligonucleotide
gtttaagagctaagctggaaacagcatagcaagtttaaataaggctagtccgttatcaatttgaaaaaatggcaccgagtcggtgct

SEQ ID NO: 439 - DNA, 87 bp, Artificial Sequence
  Description of Artificial Sequence Synthetic oligonucleotide
gtttaagagctaagctggaaacagcatagcaagtttaaataaggctagtccgttatcaaattgaaaaattggctccgagtcggagct

SEQ ID NO: 440 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcacagagtctgtgct

SEQ ID NO: 441 - DNA, 87 bp, Artificial Sequence
  Description of Artificial Sequence Synthetic oligonucleotide
gtttaagagctaagctggaaacagcatagcaagtttaaataaggctggtccgttatcaacttgaaaaagtggcaccgagtcggtgct

SEQ ID NO: 442 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcgccaagttggcgct

SEQ ID NO: 443 - DNA, 87 bp, Artificial Sequence
  Description of Artificial Sequence Synthetic oligonucleotide
gtttaagagctaagctgcaaacagcatagcaagtttaaataaggctagtccgttatcaacttgaaaaagtggcaccgagtcggtgct

SEQ ID NO: 444 - DNA, 87 bp, Artificial Sequence
  Description of Artificial Sequence Synthetic oligonucleotide
gtttaagagctaagctggaaacagcatagcaagtttaaataaggcgagtccgttatcaacttgaaaaagtggcaccgagtcggtgct

SEQ ID NO: 445 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaagtggcaccgagtcggtgct

SEQ ID NO: 446 - DNA, 87 bp, Artificial Sequence
  Description of Artificial Sequence Synthetic oligonucleotide
gtttaagagctaagctggaaacagcatagcaagtttaaataaggctagtccgttatcaatttgaaaaaatgggaccgagtcggtcct

SEQ ID NO: 447 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggcgct

SEQ ID NO: 448 - DNA, 87 bp, Artificial Sequence
  Description of Artificial Sequence Synthetic oligonucleotide
gtttaagagctaagctggaaacagcatagcaagtttaaataaggctagtccgttatcaacttgaaaatgtggcaccgagtcggtgct

SEQ ID NO: 449 - DNA, 87 bp, Artificial Sequence
  Description of Artificial Sequence Synthetic oligonucleotide
gtttaagagctaagctggaaacagcatagcaagtttaaataaggctagtccgttatcaaattgaaaaattggcaccgagtcggtgct

SEQ ID NO: 450 - DNA, 87 bp, Artificial Sequence
  Description of Artificial Sequence Synthetic oligonucleotide
gtttaagagctaagctggaaacagcatagcaagtttaaataaggttaatccgttatcaatttgaaaaaatggcgccgagtcggcgct

SEQ ID NO: 451 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcaccaagttggtgct

SEQ ID NO: 452 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggtaccgagtcggtgct

SEQ ID NO: 453 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcactgagtcagtgct

SEQ ID NO: 454 - DNA, 87 bp, Artificial Sequence
  Description of Artificial Sequence Synthetic oligonucleotide
gtttaagagctaagctggaaacagcatagcaagtttaaataaggctagaccgttatcaacttgagaaagtggcaccgagtcggtgct

SEQ ID NO: 455 - DNA, 87 bp, Artificial Sequence
  Description of Artificial Sequence Synthetic oligonucleotide
gtttaagagctaagctggaaacagcatagcaagtttaaataaggctagtccgttatcaacttgaggaagtggcaccgagtcggtgct

SEQ ID NO: 456 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcatcgagtcgatgct

SEQ ID NO: 457 - DNA, 87 bp, Artificial Sequence
  Description of Artificial Sequence Synthetic oligonucleotide
gtttaagagctaagctggaaaaagcatagcaagtttaaataaggctagtccgttatcaacttgaaaaagtggcaccgagtcggtgct

SEQ ID NO: 458 - DNA, 87 bp, Artificial Sequence
  Description of Artificial Sequence Synthetic oligonucleotide
gtttaagagctaagctggaaacagcatagcaagtttaaataaggctagtccgttatcatcttgaaaaagaggccccgagtcggggct

SEQ ID NO: 459 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacctagtaggtgct

SEQ ID NO: 460 - DNA, 87 bp, Artificial Sequence
  Description of Artificial Sequence Synthetic oligonucleotide
gtttaagagctaagctggaaacagcatagcaagtttaaataaggctggtccgttatcaacttgataaagtggcaccgagtcggtgct

SEQ ID NO: 461 - DNA, 87 bp, Artificial Sequence
  Description of Artificial Sequence Synthetic oligonucleotide
gtttaagagctaagctggaaacagcatagcaagtttaaataaggctagtccgttatcagcctgaaaaggcggcacctagtaggtgct

SEQ ID NO: 462 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggtgat

SEQ ID NO: 463 - DNA, 87 bp, Artificial Sequence
  Description of Artificial Sequence Synthetic oligonucleotide
gtttaagagctaagctggaaacagcatagcaagtttaaataaggctagtccgttatcaacttgaaaacgtggcaccgagtcggtgct

SEQ ID NO: 464 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctccaagttggagct

SEQ ID NO: 465 - DNA, 87 bp, Artificial Sequence
  Description of Artificial Sequence Synthetic oligonucleotide
gtttaagagctaagctggaaacagcatagcaagtttaaataaggctagtccgttatcaactagaaatagtggcactgagtcagtgct

SEQ ID NO: 466 - DNA, 87 bp, Artificial Sequence
  Description of Artificial Sequence Synthetic oligonucleotide
gtttaagagctaagctggaaacagcatagcaagtttaaataaggctagtccgtcagcaacttgaaaaagtggcaccgactcggtgct

SEQ ID NO: 467 - DNA, 87 bp, Artificial Sequence
  Description of Artificial Sequence Synthetic oligonucleotide
gtttaagagctaagccggaaacagcatagcaagtttaaataaggctagtccgttatcaacttgaaaaagtggcaccgagtcggtgct

SEQ ID NO: 468 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgcgtcggtgct

SEQ ID NO: 469 - DNA, 87 bp, Artificial Sequence
  Description of Artificial Sequence Synthetic oligonucleotide
gtttaagagctaagctggaaacagcatagcaagtttaaataaggctagaccgttatcaacttgaacaagtggcaccgagtcggtgct

SEQ ID NO: 470 - DNA, 87 bp, Artificial Sequence
  Description of Artificial Sequence Synthetic oligonucleotide
gtttaagagctaagctggaaacagcatagcaagtttaaataaggctagtccgttatcaaattgagaaattggcaccgagtcggtgct

SEQ ID NO: 471 - DNA, 87 bp, Artificial Sequence
  Description of Artificial Sequence Synthetic oligonucleotide
gtttaagagctaagctggaaacagcatagcaagtttaaataaggctagtccgttgccaacttgaaaaagtggcaccgactcggtgct

SEQ ID NO: 472 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgccatcgagtcgatggt

SEQ ID NO: 473 - DNA, 87 bp, Artificial Sequence
  Description of Artificial Sequence Synthetic oligonucleotide
gtttaagagctaagctggaaacagcatagcaagtttaaataaggctagtccgttatcaactcgaaagagtggccctgagtcagggct

SEQ ID NO: 474 - DNA, 87 bp, Artificial Sequence
  Description of Artificial Sequence Synthetic oligonucleotide
gtttaagagctaagctcgaaacagcatagcaagtttaaataaggctagtccgttatcaacttgaaaaagtggcaccgagtcggtgct

SEQ ID NO: 475 - DNA, 87 bp, Artificial Sequence
  Description of Artificial Sequence Synthetic oligonucleotide
gtttaagagctaagctggaaacagcatggcaagtttaaatagggctagtccgttctcaacttgaaaaagtggcaccgagtcggtgct

SEQ ID NO: 476 - DNA, 87 bp, Artificial Sequence
  Description of Artificial Sequence Synthetic oligonucleotide
gtttaagagctaagcttgaaacagcatagcaagtttaaataaggctagtccgttatcaacttgaaaaagtggcaccgagtcggtgct

SEQ ID NO: 477 - DNA, 87 bp, Artificial Sequence
  Description of Artificial Sequence Synthetic oligonucleotide
gtttaagagctaagctggaaacagcatagcaagtttaaataaggctagtccgttatcaactggaaacagtggcacctagtaggtgct

SEQ ID NO: 478 - DNA, 87 bp, Artificial Sequence
  Description of Artificial Sequence Synthetic oligonucleotide
gtttaagagctaagctggaaacagcatagcaagtttaaataaggctagtccgttatcaatttgagaaaatggcaccgagtcggtgct

SEQ ID NO: 479 - DNA, 87 bp, Artificial Sequence
  Description of Artificial Sequence Synthetic oligonucleotide
gtttaagagctaagctgaaaacagcatagcaagtttaaataaggctagtccgttatcaacttgaaaaagtggcaccgagtcggtgct

SEQ ID NO: 480 - DNA, 87 bp, Artificial Sequence
  Description of Artificial Sequence Synthetic oligonucleotide
gtttaagagctaagctggaaacagcatagcaggtttaaataaggctagtccggaatcaacttgaaaaagtggcaccgagtcggtgct

SEQ ID NO: 481 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcaccgtgtcggtgct

SEQ ID NO: 482 - DNA, 87 bp, Artificial Sequence
  Description of Artificial Sequence Synthetic oligonucleotide
gtttaagagctaagctggaaacagcatagcaagtttaaataaggctagtccgttatcaccttgaaaaaggggcacggagtccgtgct

SEQ ID NO: 483 - DNA, 87 bp, Artificial Sequence
  Description of Artificial Sequence Synthetic oligonucleotide
gtttaagagctaagctggaaacagcatagcaagtttaaataaggctagtccgttatcaacttgataaagtggcaccgagtcggtgcg

SEQ ID NO: 484 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcactgagtcagtgct

SEQ ID NO: 485 - DNA, 87 bp, Artificial Sequence
  Description of Artificial Sequence Synthetic oligonucleotide
gtttaagagctaagctggaaacagcatagcaagtttaaataaggctagtccgttatcaacatgaaaaagtggcaccgagtcggtgct

SEQ ID NO: 486 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtaaccgagtcggttat

SEQ ID NO: 487 - DNA, 87 bp, Artificial Sequence
  Description of Artificial Sequence Synthetic oligonucleotide
gtttaagagctaagctggaaacagcatagcaagtttaaataaggccagtccgttatcaacttgaaaaagtggcaccgagtcggtgct

SEQ ID NO: 488 - DNA, 87 bp, Artificial Sequence
  Description of Artificial Sequence Synthetic oligonucleotide
gtttaagagctaagctggaaacagcatagcaagtttaaataaggctagtccgttatcaccttgaaaaagggggacctagtaggtcct

SEQ ID NO: 489 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcccagagtctgggct

SEQ ID NO: 490 - DNA, 87 bp, Artificial Sequence
  Description of Artificial Sequence Synthetic oligonucleotide
gtttaagagctaagctggaaacagcatagcaagtttaaataaggttaatccgttatcaacttgaaaaagtggcgacgagtcgtcgct

SEQ ID NO: 491 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggaacggagtccgttct

SEQ ID NO: 492 - DNA, 87 bp, Artificial Sequence
  Description of Artificial Sequence Synthetic oligonucleotide
gtttaagagctaagctggaaagagcatagcaagtttaaataaggctagtccgttatcaacttgaaaaagtggcaccgagtcggtgct

SEQ ID NO: 493 - DNA, 87 bp, Artificial Sequence
  Description of Artificial Sequence Synthetic oligonucleotide
gtttaagagctaagctggaaacagcatagcaagtttaaataaggctagtccgttatcatcttgaaaaagaggcaccaagttggtgct

SEQ ID NO: 494 - DNA, 87 bp, Artificial Sequence
  Description of Artificial Sequence Synthetic oligonucleotide
gtttaagagctaagctggaaacagcatagcaagtttaaataaggctcgtccgttatcaacttgaaaaagtggcaccgagtcggtgct

SEQ ID NO: 495 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggggct

SEQ ID NO: 496 - DNA, 87 bp, Artificial Sequence
  Description of Artificial Sequence Synthetic oligonucleotide
gtttaagagctaagctggaaaccgcatagcaagtttaaataaggctagtccgttatcaacttgaaaaagtggcaccgagtcggtgct

SEQ ID NO: 497 - DNA, 87 bp, Artificial Sequence
  Description of Artificial Sequence Synthetic oligonucleotide
gtttaagagctaagctggaaacagcataacaagtttaaataaggctagtccgttatcaacttgaaaaagtggcaccgagtcggtgct

SEQ ID NO: 498 - DNA, 87 bp, Artificial Sequence
  Description of Artificial Sequence Synthetic oligonucleotide
gtttaagagctaagctggaaacagcatagcaagtttaaataaggctagtccggtatcaacttgaaaaagtggcaccgagtcggtgct

SEQ ID NO: 499 - DNA, 87 bp, Artificial Sequence
  Description of Artificial Sequence Synthetic oligonucleotide
gtttaagagctaagctggaaacagcatagcaagtttaaataaggccagtccgttatcaacttgagaaagtggcaccgagtcggtgct

SEQ ID NO: 500 - DNA, 87 bp, Artificial Sequence
  Description of Artificial Sequence Synthetic oligonucleotide
gtttaagagctaagctggaaacagcatagcaagtttaaataaggctagtccgttatcaacttgacaaagtggcaccgcgtcggtgct

SEQ ID NO: 501 - DNA, 87 bp, Artificial Sequence
  Description of Artificial Sequence Synthetic oligonucleotide
gtttaagagctaagctggaaacagcatagcaagtttaaataagactagttcgttatcaactcgaaagagtggtaccgagtcggtact

SEQ ID NO: 502 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgccaccgagtcggtgct

SEQ ID NO: 503 - DNA, 87 bp, Artificial Sequence
  Description of Artificial Sequence Synthetic oligonucleotide
gtttaagagctaagctggaaactgcatagcaagtttaaataaggctagtccgttatcaacttgaaaaagtggcaccgagtcggtgct

SEQ ID NO: 504 - DNA, 87 bp, Artificial Sequence
  Description of Artificial Sequence Synthetic oligonucleotide
gtttaagagctaagctggaaacagcatagcaagtttaaataaggctagtccggtatcaacttgtaaaagtggcaccgagtcggtgct

SEQ ID NO: 505 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaagtggcaccgactcggtgct

SEQ ID NO: 506 - DNA, 87 bp, Artificial Sequence
  Description of Artificial Sequence Synthetic oligonucleotide
gtttgagagctaagctggaaacagcatagcaagtttaaataaggctagtccgttatcaacttgaaaaagtggcaccgagtcggtgct

SEQ ID NO: 507 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacggagtccgtgct

SEQ ID NO: 508 - DNA, 87 bp, Artificial Sequence
  Description of Artificial Sequence Synthetic oligonucleotide
gtttaagagctaagctggaaacagcatagcaagtttaaataaggctagtccgatatcaacttgcaaaagtggcaccgagtcggtgct

SEQ ID NO: 509 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaaagtggcaccgagtcggtgct

SEQ ID NO: 510 - DNA, 87 bp, Artificial Sequence
  Description of Artificial Sequence Synthetic oligonucleotide
gtttaagagctaagctggaaacagcatatcaagtttaaataaggctagtccgtgatcaacttgtaaaagtggcaccgagtcggtgct

SEQ ID NO: 511 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagaggcaccgagtcggtgct

SEQ ID NO: 512 - DNA, 87 bp, Artificial Sequence
  Description of Artificial Sequence Synthetic oligonucleotide
gtttaagagctaagctggaaacagcatagcaagtttaaatagggctagtccgttatcaacttgaaaaagtggcaccgagtcggtgct

SEQ ID NO: 513 - DNA, 87 bp, Artificial Sequence
  Description of Artificial Sequence Synthetic oligonucleotide
gtttaagagctaagctggaaacagcatagcaagtttaaataaggttaatccgttatcaagttgaaaaactggcactgagtcagtgct

SEQ ID NO: 514 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggtggt

SEQ ID NO: 515 - DNA, 87 bp, Artificial Sequence
  Description of Artificial Sequence Synthetic oligonucleotide
gtttaagagctaagctggaaacagcatagcgagtttaaataaggctagtccgtcatcaacttgaaaaagtggcaccgcgtcggtgct

SEQ ID NO: 516 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctccgagtcggtgct

SEQ ID NO: 517 - DNA, 87 bp, Artificial Sequence
  Description of Artificial Sequence Synthetic oligonucleotide
gtttaagagctaagctggaaacagcatagcaagtttaaataaggcgagtccgttatcaacttgagaaagtggcaccgagtcggtgct

SEQ ID NO: 518 - DNA, 87 bp, Artificial Sequence
  Description of Artificial Sequence Synthetic oligonucleotide
gtttaagagctaagctggaaacagcatagcaagtttaaataaggctcgtccgttatcaacttgagaaagtggcaccgagtcggtgct

SEQ ID NO: 519 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaacaagttgttgct

SEQ ID NO: 520 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggtcct

SEQ ID NO: 521 - DNA, 87 bp, Artificial Sequence
  Description of Artificial Sequence Synthetic oligonucleotide
gtttaagagctaagctggaaatagcatagcaagtttaaataaggctagtccgttatcaacttgaaaaagtggcaccgagtcggtgct

SEQ ID NO: 522 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagccggtgct

SEQ ID NO: 523 - DNA, 87 bp, Artificial Sequence
  Description of Artificial Sequence Synthetic oligonucleotide
gtttaagagctaagctggaaacaacatagcaagtttaaataaggctagtccgttatcaacttgaaaaagtggcaccgagtcggtgct

SEQ ID NO: 524 - DNA, 87 bp, Artificial Sequence
  Description of Artificial Sequence Synthetic oligonucleotide
gtttaagagctaagctggaaacagcatagcaagtttaaataaggctagtccgatatcaacttgaaaaagtggcaccgagtcggtgct

SEQ ID NO: 525 - DNA, 87 bp, Artificial Sequence
  Description of Artificial Sequence Synthetic oligonucleotide
gtttaagagctaagctggaaacagcatagcaagtttaaataaggctagtccgttatcaaaatgaaaatttggcaccgagtcggtgct

SEQ ID NO: 526 - DNA, 87 bp, Artificial Sequence
  Description of Artificial Sequence Synthetic oligonucleotide
gtttaagagctaagctggaaacagcatagcaagtttaaataaggctagtccgatatcaacttgacaaagtggcaccgagtcggtgct

SEQ ID NO: 527 - DNA, 87 bp, Artificial Sequence
  Description of Artificial Sequence Synthetic oligonucleotide
gtttaagagctaagctggaaacagcatagcaagtttaaataaggccagtccgttatcaacttgacaaagtggcaccgagtcggtgct

SEQ ID NO: 528 - DNA, 87 bp, Artificial Sequence
  Description of Artificial Sequence Synthetic oligonucleotide
gtttaagagctaagctagaaacagcatagcaagtttaaataaggctagtccgttatcaacttgaaaaagtggcaccgagtcggtgct

SEQ ID NO: 529 - DNA, 87 bp, Artificial Sequence
  Description of Artificial Sequence Synthetic oligonucleotide
gtttaagagctaagctggaaacagcatagcaagtttaaataaggctagtccggtatcaacttggaaaagtggcaccgagtcggtgct

SEQ ID NO: 530 - DNA, 87 bp, Artificial Sequence
  Description of Artificial Sequence Synthetic oligonucleotide
gtttaagagctaagctggaaacagcatagcaagtttaaataagactagttcgttatcaacttgaaaaagtggcaccgagtcggtgct

SEQ ID NO: 531 - DNA, 87 bp, Artificial Sequence
  Description of Artificial Sequence Synthetic oligonucleotide
gtttaagagctaagctggaaacagcatagcaagtttaaataagactagttcgttatcagcttgaaaaagcggcatcgagtcgatgct

SEQ ID NO: 532 - DNA, 87 bp, Artificial Sequence
  Description of Artificial Sequence Synthetic oligonucleotide
gtttaagagctaagctggaaacagcatagcaagtttaaataagactagttcgttatcaacttgaaaaagtggtaccgagtcggtact

SEQ ID NO: 533 - DNA, 87 bp, Artificial Sequence
  Description of Artificial Sequence Synthetic oligonucleotide
gtttaagagctaagctggaaacagcatagcaagtttaaataaggctggtccgttatcaacttgaaaaagtggcaccgtgtcggtgct

SEQ ID NO: 534 - DNA, 87 bp, Artificial Sequence
  Description of Artificial Sequence Synthetic oligonucleotide
gtttaagagctaagctggaaacagcatagcaagtttaaataaggctagtccgttatcatcttgaaaaagtggcaccgagtcggtgct

SEQ ID NO: 535 - DNA, 87 bp, Artificial Sequence
  Description of Artificial Sequence Synthetic oligonucleotide
gtttaagagctaagctggaaacagcatatcaagtttaaataaggctagtccgttatcaacttgaaaaagtggcaccgagtcggtgct

SEQ ID NO: 536 - DNA, 87 bp, Artificial Sequence
  Description of Artificial Sequence Synthetic oligonucleotide
gtttaagagctaagctggaaacagcatagcaagtttaaataagactagttcgttatcagcttgaaaaagcggtaccgagtcggtact

SEQ ID NO: 537 - DNA, 87 bp, Artificial Sequence
  Description of Artificial Sequence Synthetic oligonucleotide
gtttaagagctaagctggaaacagcataccaagtttaaataaggctagtccgttatcaacttgaaaaagtggcaccgagtcggtgct

SEQ ID NO: 538 - DNA, 87 bp, Artificial Sequence
  Description of Artificial Sequence Synthetic oligonucleotide
gtttaagagctaagctggaaacagcatagcaagtttaaataaggctagtccgttatcaacttggaaaagttgcaccgagtcggtgct

SEQ ID NO: 539 - DNA, 87 bp, Artificial Sequence
  Description of Artificial Sequence Synthetic oligonucleotide
gtttaagagctaagctggaaacagcatagcaagtttaaataagactagttcgttatcaacttgaaaaagtgggaccgagtcggtcct

SEQ ID NO: 540 - DNA, 87 bp, Artificial Sequence
  Description of Artificial Sequence Synthetic oligonucleotide
gtttaagagctaagctggaaacagcatagcaagtttaaataaggctagtccgttatcaacttgataaagtggcaccgagccggtgct

SEQ ID NO: 541 - DNA, 87 bp, Artificial Sequence
  Description of Artificial Sequence Synthetic oligonucleotide
gtttaagagctaagctggaaacagcatagcaagtttaaataaggttaatccgttatcaatttgaaaaaatggcatcgagtcgatgct

SEQ ID NO: 542 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgacaacgagtcgttgtt

SEQ ID NO: 543 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagacggtgct

SEQ ID NO: 544 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcaccgagccggtgct

SEQ ID NO: 545 - DNA, 87 bp, Artificial Sequence
  Description of Artificial Sequence Synthetic oligonucleotide
gtttaagagctaagctggaaacagcatagcaagtttaaataaggctagtccgttatcatattgaaaaataggcaccgagtcggtgct

SEQ ID NO: 546 - DNA, 87 bp, Artificial Sequence
  Description of Artificial Sequence Synthetic oligonucleotide
gtttaagagctaagctggaaacagcatagcaagtttaaatagggctagtccgttatcaacttgagaaagtggcaccgagtcggtgct

SEQ ID NO: 547 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcacggagtccgtgct

SEQ ID NO: 548 - DNA, 87 bp, Artificial Sequence
  Description of Artificial Sequence Synthetic oligonucleotide
gtttaagagctaagctggaaacaggatagcaagtttaaataaggctagtccgttatcaacttgaaaaagtggcaccgagtcggtgct

SEQ ID NO: 549 - DNA, 87 bp, Artificial Sequence
  Description of Artificial Sequence Synthetic oligonucleotide
gtttaagagctaagctggaaacagcatagcaagtttaaataaggcgagtccgttatcaacttgaaaaagtggcaccgagtcggtgcg

SEQ ID NO: 550 - DNA, 87 bp, Artificial Sequence
  Description of Artificial Sequence Synthetic oligonucleotide
gtttaagagctaagctggaaacagcatagcaagtttaaataaggctagtccgttatcaacttgacaaagtggcaccgagccggtgct

SEQ ID NO: 551 - DNA, 87 bp, Artificial Sequence
  Description of Artificial Sequence Synthetic oligonucleotide
gtttaagagctaagctggaaacagcatagcaggtttaaataaggctagtccgttaccaacttgaaaaagtagcaccgagtcggtgct

SEQ ID NO: 552 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagttgcaccgagtcggtgct

SEQ ID NO: 553 - DNA, 87 bp, Artificial Sequence
  Description of Artificial Sequence Synthetic oligonucleotide
gtttaagagctaagctggaaacagcatagcaagtttaaataagcctagtgcgttatcaacttgaaaaagtggcaccgagtcggtgct

SEQ ID NO: 554 - DNA, 87 bp, Artificial Sequence
  Description of Artificial Sequence Synthetic oligonucleotide
gtttaagagctaagctggaaacagcatagccagtttaaataaggctagtccggtatcaacttgaaaaagtggcaccgagtcggtgct

SEQ ID NO: 555 - DNA, 87 bp, Artificial Sequence
  Description of Artificial Sequence Synthetic oligonucleotide
gtttaagagctaagctggaaacagcatagcaagtttaaataagcctagtgcgttatcaacctgaaaaggtggcatcgagtcgatgct

SEQ ID NO: 556 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggagct

SEQ ID NO: 557 - DNA, 87 bp, Artificial Sequence
  Description of Artificial Sequence Synthetic oligonucleotide
gtttaagagctaagctggaaacagcatagcaagtctaaataaggctagtccgttatcaacttgaaaaagtggcaccgagtcggtgct

SEQ ID NO: 558 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtgacctagtaggtcat

SEQ ID NO: 559 - DNA, 87 bp, Artificial Sequence
  Description of Artificial Sequence Synthetic oligonucleotide
gtttaagagctaagctggaaacagcatagaaagtttaaataaggctagtccgttatcaacttgaaaaagtggcaccgagtcggtgct

SEQ ID NO: 560 - DNA, 87 bp, Artificial Sequence
  Description of Artificial Sequence Synthetic oligonucleotide
gtttaagagctaagctggaaacagcatagcaagtttaaataagcctagtgcgttatcaacttgaaaaagtggcaacgagtcgttgct

SEQ ID NO: 561 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacagagtcggtgct

SEQ ID NO: 562 - DNA, 87 bp, Artificial Sequence
  Description of Artificial Sequence Synthetic oligonucleotide
gtttaagagctaagctggaaacagcatagcaagtttaaataagactagttcgttatcaacttgagaaagtggcaccgagtcggtgct

SEQ ID NO: 563 - DNA, 87 bp, Artificial Sequence
  Description of Artificial Sequence Synthetic oligonucleotide
gtttaagagctaagctggaaacagcattgcaagtttaaataaggcgagtccgttatcaacttgcaaaagtggcaccgagtcggtgct

SEQ ID NO: 564 - DNA, 87 bp, Artificial Sequence
  Description of Artificial Sequence Synthetic oligonucleotide
gtttaagagctaagctggaaacagcatagcaagtttaaataagactagttcgttatcaacgtgaaaacgtggcaccaagttggtgct

SEQ ID NO: 565 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtctgtgct

SEQ ID NO: 566 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgccagcgagtcgctggt

SEQ ID NO: 567 - DNA, 87 bp, Artificial Sequence
  Description of Artificial Sequence Synthetic oligonucleotide
gtttaagagctaagctggaaacagcatagcaagtttaaataaggcacgtccgttatcaacttgaaaaagtggcaccgaatcggtgct

SEQ ID NO: 568 - DNA, 87 bp, Artificial Sequence
  Description of Artificial Sequence Synthetic oligonucleotide
gtttaagagctaagctggaaacatcatagcaagtttaaataaggctagtccgttatcaacttgaaaaagtggcaccgagtcggtgct

SEQ ID NO: 569 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggtact

SEQ ID NO: 570 - DNA, 87 bp, Artificial Sequence
  Description of Artificial Sequence Synthetic oligonucleotide
gtttaagagctaagctggaaacagcatagcaagtttaaataagcctagtgcgttatcaacttgaaaaagtgggaccgagtcggtcct

SEQ ID NO: 571 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaaagagtctttgct

SEQ ID NO: 572 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggaaccgagtcggtgct

SEQ ID NO: 573 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtagcaccgagtcggtgct

SEQ ID NO: 574 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggccctgagtcagggct

SEQ ID NO: 575 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcagtgct

SEQ ID NO: 576 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgataccaagttggtatt

SEQ ID NO: 577 - DNA, 87 bp, Artificial Sequence
  Description of Artificial Sequence Synthetic oligonucleotide
gtttaagagctaagctggaaacagcatagcaagtttaaataagcctagtgcgttatcagcttgaaaaagcggcaccgagtcggtgct

SEQ ID NO: 578 - DNA, 87 bp, Artificial Sequence
  Description of Artificial Sequence Synthetic oligonucleotide
gtttaagagctaagctggaaacagcatagcaagtttaaataaggcgagtccgtcagcaacttgaaaaagtggcaccgagtcggtgct

SEQ ID NO: 579 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgccaccaagttggtggt

SEQ ID NO: 580 - DNA, 87 bp, Artificial Sequence
  Description of Artificial Sequence Synthetic oligonucleotide
gtttaagagctaagctggaaacagcataggaagtttaaataaggctagtccgttatcaacttgaaaaagtggcaccgagtcggtgct

SEQ ID NO: 581 - DNA, 87 bp, Artificial Sequence
  Description of Artificial Sequence Synthetic oligonucleotide
gtttaagagctaagctggaaacagcatcgcaagtttaaataaggctagtccggtatcaacttggaaaagtggcaccgagtcggtgct

SEQ ID NO: 582 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcacctagtaggtgct

SEQ ID NO: 583 - DNA, 87 bp, Artificial Sequence
  Description of Artificial Sequence Synthetic oligonucleotide
gtttaagagctaagctggaaacagcatagcaagtttaaatagggctagtccgttatcaacttgataaagtggcaccgagtcggtgct

SEQ ID NO: 584 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaattggcaccgagtcggtgct

SEQ ID NO: 585 - DNA, 87 bp, Artificial Sequence
  Description of Artificial Sequence Synthetic oligonucleotide
gtttaagagctaagctggaaacagcatagcaagtttaaataaggctagtccgttatcaaatagaaatattggcagcgagtcgctgct

SEQ ID NO: 586 - DNA, 87 bp, Artificial Sequence
  Description of Artificial Sequence Synthetic oligonucleotide
gtttaagagctaagctggaaacagcatagcaagtttaaataagcctagtgcgttatcaacttgagaaagtggcaccgagtcggtgct

SEQ ID NO: 587 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggccccgagtcggtgct

SEQ ID NO: 588 - DNA, 87 bp, Artificial Sequence
  Description of Artificial Sequence Synthetic oligonucleotide
gtttaagagctaagcgggaaacagcatagcaagtttaaataaggctagtccgttatcaacttgaaaaagtggcaccgagtcggtgct

SEQ ID NO: 589 - DNA, 87 bp, Artificial Sequence
  Description of Artificial Sequence Synthetic oligonucleotide
gtttaagagctaagctggaaacaccatagcaagtttaaataaggctagtccgttatcaacttgaaaaagtggcaccgagtcggtgct

SEQ ID NO: 590 - DNA, 87 bp, Artificial Sequence
  Description of Artificial Sequence Synthetic oligonucleotide
gtttaagagctaagctggaaacagcatagcaagtttaaataagcctagtgcgttatcaacttgaaaaagtggcagcgagtcgctgct

SEQ ID NO: 591 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctcggagtccgagct

SEQ ID NO: 592 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacaaagtttgtgct

SEQ ID NO: 593 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcactaagttagtgct

SEQ ID NO: 594 - DNA, 87 bp, Artificial Sequence
  Description of Artificial Sequence Synthetic oligonucleotide
gtttaagagctaagctggaaacagcatcgcaagtttaaataaggctggtccggtatcaacttgaaaaagtggcaccgagtcggtgct

SEQ ID NO: 595 - DNA, 87 bp, Artificial Sequence
  Description of Artificial Sequence Synthetic oligonucleotide
gtttaagagctaagctggaaacagcatagcaagtttaaataaggccagtccgtgatcaacttgaaaaagtggcaccgagccggtgct

SEQ ID NO: 596 - DNA, 87 bp, Artificial Sequence
  Description of Artificial Sequence Synthetic oligonucleotide
gtttaagagctaagctggaaacagcataccaagtttaaataaggcttgtccgttatcaacttgagaaagtggcaccgagtcggtgct

SEQ ID NO: 597 - DNA, 87 bp, Artificial Sequence
  Description of Artificial Sequence Synthetic oligonucleotide
gtttaagagctaagctggaaacagcatagcaagtttaaataaggctagtccgttatcaatttgaaaaagtggcaccgagtcggtgct

SEQ ID NO: 598 - DNA, 87 bp, Artificial Sequence
  Description of Artificial Sequence Synthetic oligonucleotide
gtttaagagctaagctggaaacagcatagcaagtttaaataaggctagtccgttatcaagaaaaaatactggcaccgagtcggtgct

SEQ ID NO: 599 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgcgtcggtgcc

SEQ ID NO: 600 - DNA, 87 bp, Artificial Sequence
  Description of Artificial Sequence Synthetic oligonucleotide
gtttaagagctaagctggaaacagcatagcaagtttaaataaggctagtccgttatcaaattgaaaaattggtaacgagtcgttact

SEQ ID NO: 601 - DNA, 87 bp, Artificial Sequence
  Description of Artificial Sequence Synthetic oligonucleotide
gtttaagagctaagctggaaacagcatagcaagtttaaataaggctagtccgatgtcaacttgtaaaagtggcaccgagtcggtgct

SEQ ID NO: 602 - DNA, 87 bp, Artificial Sequence
  Description of Artificial Sequence Synthetic oligonucleotide
gtttaagagctaagcaggaaacagcatagcaagtttaaataaggctagtccgttatcaacttgaaaaagtggcaccgagtcggtgct

SEQ ID NO: 603 - DNA, 87 bp, Artificial Sequence
  Description of Artificial Sequence Synthetic oligonucleotide
gtttaagagctaagctggaaacagcatagccagtttaaataaggctcgtccgttatcaacttcaaaaagtggcaccgagtcggtgct

SEQ ID NO: 604 - DNA, 87 bp, Artificial Sequence
  Description of Artificial Sequence Synthetic oligonucleotide
gtttaagagctaagctggaaacagcatagcaagtttaaataaggctagttcgttatcaacttgaaaaagtggcaccgagtcggtgct

SEQ ID NO: 605 - DNA, 87 bp, Artificial Sequence
  Description of Artificial Sequence Synthetic oligonucleotide
gtttaagagctaagctggaaacagcattgcaagtttaaatagggctagtccgttatcaacttgaaaaagtggcaccgattcggtgct

SEQ ID NO: 606 - DNA, 87 bp, Artificial Sequence
  Description of Artificial Sequence Synthetic oligonucleotide
gtttaagagctaagctggaaacagcatagcaagtttaaataaggctagtccgttatcacctggaaacaggggcacccagtgggtgct

SEQ ID NO: 607 - DNA, 87 bp, Artificial Sequence
  Description of Artificial Sequence Synthetic oligonucleotide
gtttaagagctaagctggaaacagcattgcaagtttaaatagggctagtccgctatcaacttgaaaaagtggcaccgagtcggtgct

SEQ ID NO: 608 - DNA, 87 bp, Artificial Sequence
  Description of Artificial Sequence Synthetic oligonucleotide
gtttaagagctaagctggaaacagcatagcaagtttaaataaggctagcccgttatcaacttgaaaaagtggcaccgaatcggtgct

SEQ ID NO: 609 - DNA, 87 bp, Artificial Sequence
  Description of Artificial Sequence Synthetic oligonucleotide
gtttaagagctaagctggaaacagcatagcaagtttaaataagtctagtacgttatcaacttgaaaaagtgctaccgagtcggtagt

SEQ ID NO: 610 - DNA, 87 bp, Artificial Sequence
  Description of Artificial Sequence Synthetic oligonucleotide
gtttaagagctaagctggaaacagcatagcaaatttaaataaggctagtccgctatcaacttgaaaaagtggcaccgagtcggtgct

SEQ ID NO: 611 - DNA, 87 bp, Artificial Sequence
  Description of Artificial Sequence Synthetic oligonucleotide
gtttaagagctaagctggaaacagcatagcaagtttaaataaggctagcccgttatcaacttgaaaaagtggcaccgagtcggtgct

SEQ ID NO: 612 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacctagtcggtgct

SEQ ID NO: 613 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcggttct

SEQ ID NO: 614 - DNA, 87 bp, Artificial Sequence
  Description of Artificial Sequence Synthetic oligonucleotide
gtttaagagctaagctggaaacagcatagcaagtttaaataagactagttcgttatcaaattgaaaaattggcaccgagtcggtgct

SEQ ID NO: 615 - DNA, 87 bp, Artificial Sequence
  Description of Artificial Sequence Synthetic oligonucleotide
gtttaagagctaagctggaaacagcatagcaagtttaaataagtctagtacgttatcaacttgaaaaagtggcaccgagtcggtgct

SEQ ID NO: 616 - DNA, 87 bp, Artificial Sequence
  Description of Artificial Sequence Synthetic oligonucleotide
gtttaagagctaagctggaaacagcatagcaagtttaaataagcctagtgcgttatcaacgtgaaaacgtggcacggagtccgtgct

SEQ ID NO: 617 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgggtcggtgct

SEQ ID NO: 618 - DNA, 87 bp, Artificial Sequence
  Description of Artificial Sequence Synthetic oligonucleotide
gtttaagagctaagctggaaacagcatagcaagtttaaataaggatattccgttatcaactcgaaagagtggcaccgagtcggtgct

SEQ ID NO: 619 - DNA, 87 bp, Artificial Sequence
  Description of Artificial Sequence Synthetic oligonucleotide
gtttaagagctaagctggaaacagcatagcaagtttaaataagtctagtacgttatcaattggaaacaatggcaccgagtcggtgct

SEQ ID NO: 620 - DNA, 87 bp, Artificial Sequence
  Description of Artificial Sequence Synthetic oligonucleotide
gtttaagagctaagctggaaacagcatagcaaatttaaataaggctagtccgttatcaacttgaaaaagtggcaccgagtcggtgct

SEQ ID NO: 621 - DNA, 87 bp, Artificial Sequence
  Description of Artificial Sequence Synthetic oligonucleotide
gtttaagagctaagctggaaacagcattgcaagtttaaataaggctagtccgttgtcaacttgaaaaagtggcaccgagccggtgct

SEQ ID NO: 622 - DNA, 87 bp, Artificial Sequence
  Description of Artificial Sequence Synthetic oligonucleotide
gtttacgagctaagctggaaacagcatagcaagtttaaataaggctagtccgttatcaacttgaaaaagtggcaccgagtcggtgct

SEQ ID NO: 623 - DNA, 87 bp, Artificial Sequence
  Description of Artificial Sequence Synthetic oligonucleotide
gtttaagagctaagctggaaacagcatagcaagtttaaataaggctagtccgttatcaacttggaaaagtggcaccgggtcggtgct

SEQ ID NO: 624 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacggagtcggtgct

SEQ ID NO: 625 - DNA, 87 bp, Artificial Sequence
  Description of Artificial Sequence Synthetic oligonucleotide
gtttaagagctaagctggaaacagcatcgcaagtttaaataaggctagtccgttctcaacttgaaaaagtcgcaccgagtcggtgct

SEQ ID NO: 626 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtcgcaccgagtcggtgct

SEQ ID NO: 627 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggaccgagtcggtgct

SEQ ID NO: 628 - DNA, 87 bp, Artificial Sequence
  Description of Artificial Sequence Synthetic oligonucleotide
gtttaagagctaagctggaaacagcatagcaagtttaaataaggatattccgttatcaattcgaaagaatggcaccgagtcggtgct

SEQ ID NO: 629 - DNA, 87 bp, Artificial Sequence
  Description of Artificial Sequence Synthetic oligonucleotide
gtttaagagctaagctggaaacagcatagcaagtttaaataaggctagtccgatggcaacttgaaaaagtggcaccgagtcggtgct

SEQ ID NO: 630 - DNA, 87 bp, Artificial Sequence
  Description of Artificial Sequence Synthetic oligonucleotide
gtttaagagctaagctggaaacagcatagcaagtttaaataaggctagtccgttatcaacctgaaaaggtgccacggagtccgtggt

SEQ ID NO: 631 - DNA, 87 bp, Artificial Sequence
  Description of Artificial Sequence Synthetic oligonucleotide
gtttaagagctaagctggaaacagcatagcaagtttaaataaggctagtccgttatcaacgtgaaaacgtggcacacagtgtgtgct

SEQ ID NO: 632 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgaaccggtgct

SEQ ID NO: 633 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagccggtgcc

SEQ ID NO: 634 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggacccagtgggtcct

SEQ ID NO: 635 - DNA, 87 bp, Artificial Sequence
  Description of Artificial Sequence Synthetic oligonucleotide
gtttaagagctaagctggaaacagcatagcaagtttaaataagtctagtacgttatcaacttgagaaagtggcaccgagtcggtgct

SEQ ID NO: 636 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcaccgggtcggtgct

SEQ ID NO: 637 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtagcaccgagtcggtgct

SEQ ID NO: 638 - DNA, 87 bp, Artificial Sequence
  Description of Artificial Sequence Synthetic oligonucleotide
gtttaagagctaagctggaaacagcatagcaagtttaaataaggcaagcccgttatcaacttgaaaaagtggcaccgagtcggtgct

SEQ ID NO: 639 - DNA, 87 bp, Artificial Sequence
  Description of Artificial Sequence Synthetic oligonucleotide
gtttaagagctaagctggaaacagcatagcaagtttaaataagggtactccgttatcaacgcgaaagcgtggcaccgagtcggtgct

SEQ ID NO: 640 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcgatgct

SEQ ID NO: 641 - DNA, 87 bp, Artificial Sequence
  Description of Artificial Sequence Synthetic oligonucleotide
gtttaagagctaagctggaaacagcatagcaagcttaaataaggctagtccgttatcaactcgaaagagtggcaccgagtcggtgct

SEQ ID NO: 642 - DNA, 87 bp, Artificial Sequence
  Description of Artificial Sequence Synthetic oligonucleotide
gtttaagagctaagctggaaacagcatagcaagtttaaataaggttaatccgttatcaacttgaaaaagtggcatctagtagatgct

SEQ ID NO: 643 - DNA, 87 bp, Artificial Sequence
  Description of Artificial Sequence Synthetic oligonucleotide
gtttaagagctaagctggaaacagcatagccagtttaaataaggctagtccgttatcaacttgaaaaagttgcaccgagtcggtgct

SEQ ID NO: 644 - DNA, 87 bp, Artificial Sequence
  Description of Artificial Sequence Synthetic oligonucleotide
gtttaggagctaagctggaaacagcatagcaagtttaaataaggctagtccgttatcaacttgaaaaagtggcaccgagtcggtgct

SEQ ID NO: 645 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggcattgagtcaatgct

SEQ ID NO: 646 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcgttgct

SEQ ID NO: 647 - DNA, 87 bp, Artificial Sequence
  Description of Artificial Sequence Synthetic oligonucleotide
gtttaagagctaagctggaaacagcatagcaagtttaaacaaggctagtccgttatcaacttgaaaaagtggcaccgagtcggtgct

SEQ ID NO: 648 - DNA, 87 bp, Artificial Sequence
  Description of Artificial Sequence Synthetic oligonucleotide
gtttaagagctaagctggaaacagcataacaagtttaaatagggctagtccgttatcaacttgagaaagtggcaccgagtcggtgct

SEQ ID NO: 649 - DNA, 87 bp, Artificial Sequence
  Description of Artificial Sequence Synthetic oligonucleotide
gtttaagagctaagctggaaacagcatagcaagtttaaataaggctagtccgttagcaacttgaaaaagtagcaccgagtcggtgct

SEQ ID NO: 650 - DNA, 87 bp, Artificial Sequence
  Description of Artificial Sequence Synthetic oligonucleotide
gtttgatagctaagctggaaacagcatagcaagtttaaataaggctagtccgttatcaacttgaaaaagtggcaccgagtcggtgct

SEQ ID NO: 651 - DNA, 87 bp, Artificial Sequence
  Description of Artificial Sequence Synthetic oligonucleotide
atttaagagctaagctggaaacagcatagcaagtttaaataaggctagtccgttatcaacttgaaaaagtggcaccgagtcggtgct

SEQ ID NO: 652 - DNA, 87 bp, Artificial Sequence
  Description of Artificial Sequence Synthetic oligonucleotide
gtttaagagctaagctggaaacagcatatcaagtttaaataaggctagtccggtatcaacttgaaaaagtggcaccgagtcggtgct

SEQ ID NO: 653 - DNA, 87 bp, Artificial Sequence
  Description of Artificial Sequence Synthetic oligonucleotide
gtttaagagctaagctggaaacagcatagcaaatttaaataaggctagtccgttatcaacttgacaaagtggcaccgagtcggtgct

SEQ ID NO: 654 - DNA, 87 bp, Artificial Sequence
  Description of Artificial Sequence Synthetic oligonucleotide
gtttaagagctaagctggaaacagcatagcaagtttaaataaggctagtccgttattaacttgaaaaagtggcaccgagtcggtgct

SEQ ID NO: 655 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccaagtcggtgct

SEQ ID NO: 656 - DNA, 87 bp, Artificial Sequence
  Description of Artificial Sequence Synthetic oligonucleotide
gtttaagagctaagctggaaacagcatagcaagtttaaataaggctagtccgttataaacttgaaaaagtggcaccgagtcggtgct

SEQ ID NO: 657 - DNA, 87 bp, Artificial Sequence
  Description of Artificial Sequence Synthetic oligonucleotide
gtttaagagctaagctggaaacagcatagcaagcttaaataaggctagtccgttatcaacctgaaaaggtggcaccgagtcggtgct

SEQ ID NO: 658 - DNA, 87 bp, Artificial Sequence
  Description of Artificial Sequence Synthetic oligonucleotide
gtttaagagctaagctggaaacagcatagcaagtttaaataaggctagtccgttatgaacttgaaaaagtggcaccgagtcggtgct

SEQ ID NO: 659 - DNA, 87 bp, Artificial Sequence
  Description of Artificial Sequence Synthetic oligonucleotide
gtttactagctaagctggaaacagcatagcaagtttaaataaggctagtccgttatcaacttgaaaaagtggcaccgagtcggtgct

SEQ ID NO: 660 - DNA, 87 bp, Artificial Sequence
  Description of Artificial Sequence Synthetic oligonucleotide
gtttaagagctaagctggaaacagcatagcaagtttaaataagggtactccgttatcaactcgaaagagtggcacagagtctgtgct

SEQ ID NO: 661 - DNA, 87 bp, Artificial Sequence
  Description of Artificial Sequence Synthetic oligonucleotide
gtttaagagctaagctggaaacagcatagcaagtttaaataaggctagtccgctaaccacttgaaaaagtggcaccgagtcggtgct

SEQ ID NO: 662 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtggcaccgagtcggtgct

SEQ ID NO: 663 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtggctccgagtcggagct

SEQ ID NO: 664 - DNA, 87 bp, Artificial Sequence
  Description of Artificial Sequence Synthetic oligonucleotide
gtttaagagctaagctggaaacagcatagcaaatttaaataaggctagtccgttatcaacttgaagaagtggcaccgagtcggtgct

SEQ ID NO: 665 - DNA, 87 bp, Artificial Sequence
  Description of Artificial Sequence Synthetic oligonucleotide
gtttaagagctaagctggaaacagcatagcaagtttaaataaggatattccgttatcaacatgaaaatgtggcaacgagtcgttgct

SEQ ID NO: 666 - DNA, 87 bp, Artificial Sequence
  Description of Artificial Sequence Synthetic oligonucleotide
gtttaagagctaagctggaaacagcatagcaagtttaaataaggctagtccgttatcaagttgaaaaactgggacccagtgggtcct

SEQ ID NO: 667 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacccagtgggtgct

SEQ ID NO: 668 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtcgcaccgagtcggtgct

SEQ ID NO: 669 - DNA, 87 bp, Artificial Sequence
  Description of Artificial Sequence Synthetic oligonucleotide
gtctaagagctaagctggaaacagcatagcaagtttaaataaggctagtccgttatcaacttgaaaaagtggcaccgagtcggtgct

SEQ ID NO: 670 - DNA, 87 bp, Artificial Sequence
  Description of Artificial Sequence Synthetic oligonucleotide
gtttaagagctaagctggaaacagcatagcaagtttaaataagggtactccgttatcaacttgaaaaagtgacaccgagtcggtgtt

SEQ ID NO: 671 - DNA, 87 bp, Artificial Sequence
  Description of Artificial Sequence Synthetic oligonucleotide
gtttaagagctaagctggaaacagcatagcaagcttaaataaggctagtccgttatcaactggaaacagtggcaccgagtcggtgct

SEQ ID NO: 672 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcgcccagtgggcgct

SEQ ID NO: 673 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtgataccgagtcggtatt

SEQ ID NO: 674 - DNA, 87 bp, Artificial Sequence
  Description of Artificial Sequence Synthetic oligonucleotide
gtttaagagctaagctggaaacagcatagcatgtttaaataaggctagtccattctcaacttgaaaaagtggcaccgagtcggtgct

SEQ ID NO: 675 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaactggcaccgagtcggtgct

SEQ ID NO: 676 - DNA, 87 bp, Artificial Sequence
  Description of Artificial Sequence Synthetic oligonucleotide
gtttaagagctaagctggaaacagcatagcaagtctaaataaggcgagtccgttatcaacttgaaaaagtggcaccgagtcggtgct

SEQ ID NO: 677 - DNA, 87 bp, Artificial Sequence
  Description of Artificial Sequence Synthetic oligonucleotide
gtttaagagctaagctggaaacagcatagcaaatttaaataaggcaagtccgttatcaacttgaaaaagtggcaccgagtcggtgct

SEQ ID NO: 678 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtggctacgagtcgtagct

SEQ ID NO: 679 - DNA, 87 bp, Artificial Sequence
  Description of Artificial Sequence Synthetic oligonucleotide
gtttaagagctaagctggaaacagcatagcaagtttaaataaggatattccgttatcaacttgagaaagtggcaccgagtcggtgct

SEQ ID NO: 680 - DNA, 87 bp, Artificial Sequence
  Description of Artificial Sequence Synthetic oligonucleotide
gtttaagagctaagctggaaacagcatagcaagtttaaataaggatattccgttatcaagttgaaaaactggcaccgagtcggtgct

SEQ ID NO: 681 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtgccaccgagtcggtggt

SEQ ID NO: 682 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgaggcggtgct

SEQ ID NO: 683 - DNA, 87 bp, Artificial Sequence
  Description of Artificial Sequence Synthetic oligonucleotide
gtttaagagctaagctggaaacagcatagcaagtttaaataaggctagtccgttattaacttgaacaagtggcaccgagtcggtgct

SEQ ID NO: 684 - DNA, 87 bp, Artificial Sequence
  Description of Artificial Sequence Synthetic oligonucleotide
gtttaagagctaagctggaaacagcatagcaagtttaaataagggtactccgttatcaacttgaaaaagtggcaccgagtcggtgct

SEQ ID NO: 685 - DNA, 87 bp, Artificial Sequence
  Description of Artificial Sequence Synthetic oligonucleotide
gtttaagagctaagctggaaacagcatagcaagtttaaataaggctagtccgtttgcaacttgaaaaagtagcaccgagtcggtgct

SEQ ID NO: 686 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaaatggcaccgagtcggtgct

SEQ ID NO: 687 - DNA, 87 bp, Artificial Sequence
  Description of Artificial Sequence Synthetic oligonucleotide
gtttaagagctaagctggaaacagcatagcaagtttaaataagggtactccgttatcaacttgagaaagtggcaccgagtcggtgct

SEQ ID NO: 688 - DNA, 87 bp, Artificial Sequence
  Description of Artificial Sequence Synthetic oligonucleotide
gtttaagagctaagctggaaacagcatagcaagtttaaataagggtactccgttatcaacttgaaaaagtgccaccgagtcggtggt

SEQ ID NO: 689 - DNA, 87 bp, Artificial Sequence
  Description of Artificial Sequence Synthetic oligonucleotide
gtttaagagctaagctggaaacagcatagcaagtttaaataaggctagtccgttatcagcttgaaaaagcggcggcgagtcgccgct

SEQ ID NO: 690 - DNA, 87 bp, Artificial Sequence
  Description of Artificial Sequence Synthetic oligonucleotide
gtttaagagctaagctggaaacagcatagcaagcttaaataaggctagtccgttatcagcttgaaaaagcggcaccgagtcggtgct

SEQ ID NO: 691 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacccagtcggtgct

SEQ ID NO: 692 - DNA, 87 bp, Artificial Sequence
  Description of Artificial Sequence Synthetic oligonucleotide
gtttaagagctaagctggaaacagcatggcaagtttaaataaggctagtccgttatcaacttaaaaaagtggcaccgggtcggtgct

SEQ ID NO: 693 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacacagtgtgtgct

SEQ ID NO: 694 - DNA, 87 bp, Artificial Sequence
  Description of Artificial Sequence Synthetic oligonucleotide
gtttaagagctaagctggaaacagcatagcaagtttaaataaggctagtccgttattaacttgataaagtggcaccgagtcggtgct

SEQ ID NO: 695 - DNA, 87 bp, Artificial Sequence
  Description of Artificial Sequence Synthetic oligonucleotide
gtttaagagctaagctggaaacagcatagcaagcttaaataaggctagtccgttatcaacgtgaaaacgtggcaccgagtcggtgct

SEQ ID NO: 696 - DNA, 87 bp, Artificial Sequence
  Description of Artificial Sequence Synthetic oligonucleotide
gtttaagagctaagctggaaacagcatagcaagtttaaataaggctagtccgttataaacttgaaaaagtggcaccgaatcggtgct

SEQ ID NO: 697 - DNA, 87 bp, Artificial Sequence
  Description of Artificial Sequence Synthetic oligonucleotide
gtttacgtgctaagctggaaacagcatagcaagtttaaataaggctagtccgttatcaacttgaaaaagtggcaccgagtcggtgct

SEQ ID NO: 698 - DNA, 87 bp, Artificial Sequence
  Description of Artificial Sequence Synthetic oligonucleotide
gtttaagagctaagctggaaacagcatagcaagtttaaataagactagttcgttatcaaattgaaaaattggcacagagtctgtgct

SEQ ID NO: 699 - DNA, 87 bp, Artificial Sequence
  Description of Artificial Sequence Synthetic oligonucleotide
gtttaagagctaagctggaaacagcatagcaagtttaaataaggctagtccgttatcaattcgaaatactggcaccgagtcggtgct

SEQ ID NO: 700 - DNA, 87 bp, Artificial Sequence
  Description of Artificial Sequence Synthetic oligonucleotide
gtttaagagctaagctggaaacagcattgccagtttaaataaggctagtccgttatcaacttgaaaaagtggcaccgcgtcggtgct

SEQ ID NO: 701 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggtaccgagtcggtact

SEQ ID NO: 702 - DNA, 87 bp, Artificial Sequence
  Description of Artificial Sequence Synthetic oligonucleotide
gtctaagtgctaagctggaaacagcatagcaagtttaaataaggctagtccgttatcaacttgaaaaagtggcaccgagtcggtgct

SEQ ID NO: 703 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcagcgagtcggtgct

SEQ ID NO: 704 - DNA, 87 bp, Artificial Sequence
  Description of Artificial Sequence Synthetic oligonucleotide
gtttaagagctaagctggaaacagcatagcaagtttaaataaggctagtccgttatcgacttgaaaaagtggcaccgagtcggtgct

SEQ ID NO: 705 - DNA, 87 bp, Artificial Sequence
  Description of Artificial Sequence Synthetic oligonucleotide
gtttaagagctaagctggaaacagcatagcaagtttaaataaggttagtccgttatcaacttgaaaaagtggcaccgagtcggtgct

SEQ ID NO: 706 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaacgagtcggtgct

SEQ ID NO: 707 - DNA, 87 bp, Artificial Sequence
  Description of Artificial Sequence Synthetic oligonucleotide
gtttaagagctaagctggaaacagcatagcaagtttaaataaggctagcccgttatcaacgtgaaaaagtggcaccgagtcggtgct

SEQ ID NO: 708 - DNA, 87 bp, Artificial Sequence
  Description of Artificial Sequence Synthetic oligonucleotide
gcttaagagctaagctggaaacagcatagcaagtttaaataaggctagtccgttatcaacttgaaaaagtggcaccgagtcggtgct

SEQ ID NO: 709 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtgggaccgagtcggtcct

SEQ ID NO: 710 - DNA, 87 bp, Artificial Sequence
  Description of Artificial Sequence Synthetic oligonucleotide
gtttaagagctaagctggaaacagcatagcaagcttaaataaggctagtccgttatcaccttgaaaaaggggcaccgagtcggtgct

SEQ ID NO: 711 - DNA, 87 bp, Artificial Sequence
  Description of Artificial Sequence Synthetic oligonucleotide
gtttaagagctaagctggaaacagcatagcaagtttacataaggctagtccgttatcaacttgaaaaagtggcaccgagtcggtgct

SEQ ID NO: 712 - DNA, 87 bp, Artificial Sequence
  Description of Artificial Sequence Synthetic oligonucleotide
gtttaagagctaagctggaaacagcatagcaagtttaaataaggctagtccgtaattaacttgaaaaagtggcaccgagacggtgct

SEQ ID NO: 713 - DNA, 87 bp, Artificial Sequence
  Description of Artificial Sequence Synthetic oligonucleotide
gtttaagagctaagctggaaacagcatagcaagtttaaatatggctagtccgttatcaacttgaaaaagtggcaccgagtcggtgct

SEQ ID NO: 714 - DNA, 87 bp, Artificial Sequence
  Description of Artificial Sequence Synthetic oligonucleotide
gtttaagagctaagctggaaacagcatagcaagtttaaataaggctagtccgttatcaacttgagaaagtggcacccagtgggtgct

SEQ ID NO: 715 - DNA, 87 bp, Artificial Sequence
  Description of Artificial Sequence Synthetic oligonucleotide
gtttaagagctaagctggaaacagcatagcaagtttaaataaggctagcccgatatcaacttgaacaagtggcaccgagtcggtgct

SEQ ID NO: 716 - DNA, 87 bp, Artificial Sequence
  Description of Artificial Sequence Synthetic oligonucleotide
gtttaagagctaagctggaaacagcatagcaagtttaaataaggctagtccgttatcaacttgacaaagtggcaccgaggcggtgct

SEQ ID NO: 717 - DNA, 87 bp, Artificial Sequence
  Description of Artificial Sequence Synthetic oligonucleotide
gtttaagagctaagctggaaacagcatagcaagtttaaataaggctagtccgttagaaacttgaaaaagtggcaccgaatcggtgct

SEQ ID NO: 718 - DNA, 87 bp, Artificial Sequence
  Description of Artificial Sequence Synthetic oligonucleotide
gtttaagagctaagctggaaacagcatagcaagtttaaataaggctagtccgttatcaactggaaacagtggcactcagtgagtgct

SEQ ID NO: 719 - DNA, 87 bp, Artificial Sequence
  Description of Artificial Sequence Synthetic oligonucleotide
gtttaagagctaagctggaaacagcatagcaagtttaaataaggctagtccgttatccacttgaaaaagtggcaccgagtcggtgct

SEQ ID NO: 720 - DNA, 87 bp, Artificial Sequence
  Description of Artificial Sequence Synthetic oligonucleotide
gtttatgagctaagctggaaacagcatagcaagtttaaataaggctagtccgttatcaacttgaaaaagtggcaccgagtcggtgct

SEQ ID NO: 721 - DNA, 87 bp, Artificial Sequence
  Description of Artificial Sequence Synthetic oligonucleotide
gtttaagagctaagctggaaacagcatagcaagtttaaataagggtactccgttatcaacttgaaaaagtggaactgagtcagttct

SEQ ID NO: 722 - DNA, 87 bp, Artificial Sequence
  Description of Artificial Sequence Synthetic oligonucleotide
gtttaagagctaagctggaaacagcatagcaagtttaaataagcgtactgcgttatcaacttgaaaaagtggcaccgagtcggtgct

SEQ ID NO: 723 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggaaccgagtcggttct

SEQ ID NO: 724 - DNA, 87 bp, Artificial Sequence
  Description of Artificial Sequence Synthetic oligonucleotide
gtttaagagctaagctggaaacagcatagcaagtttaaagaaggctagtccgttatcaacttgaaaaagtggcaccgagtcggtgct

SEQ ID NO: 725 - DNA, 87 bp, Artificial Sequence
  Description of Artificial Sequence Synthetic oligonucleotide
gtttaagagctaagctggaaacagcatagcatgtttaaatatggctagtccgttatcaacttgaaaaagtggcaccgagtcggtgct

SEQ ID NO: 726 - DNA, 87 bp, Artificial Sequence
  Description of Artificial Sequence Synthetic oligonucleotide
gtttaagagctaagctggaaacagcatagcaagtttaaataaggctagtccgttatcaaattgaaaaagtggcaccgagtcggtgct

SEQ ID NO: 727 - DNA, 87 bp, Artificial Sequence
  Description of Artificial Sequence Synthetic oligonucleotide
gtttacgggctaagctggaaacagcatagcaagtttaaataaggctagtccgttatcaacttgaaaaagtggcaccgagtcggtgct

SEQ ID NO: 728 - DNA, 87 bp, Artificial Sequence
  Description of Artificial Sequence Synthetic oligonucleotide
gtttaagagctaagctggaaacagcatagcaagtttaaataaggctagcccgttacaaacttgaaaaagtggcaccgagtcggtgct

SEQ ID NO: 729 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtccgtgct

SEQ ID NO: 730 - DNA, 87 bp, Artificial Sequence
  Description of Artificial Sequence Synthetic oligonucleotide
gtttaagagctaagctggaaacagcatagcaagtttaaataaggctagtccgctattaacttgaaaaagtggcaccgaatcggtgct

SEQ ID NO: 731 - DNA, 87 bp, Artificial Sequence
  Description of Artificial Sequence Synthetic oligonucleotide
gtttaagagctaagctggaaacagcatagcaagtttaaataagtctagtacgttatcaatttgaaaaaatggcaacgagtcgttgct

SEQ ID NO: 732 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcaccgagtcggtgct

SEQ ID NO: 733 - DNA, 87 bp, Artificial Sequence
  Description of Artificial Sequence Synthetic oligonucleotide
gtttaagagctaagctggaaacagcatagcaagcttaaataaggctagtccgctatcaacttgaaaaagtggcaccgagtcggtgct

SEQ ID NO: 734 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtcagcaagttgctgat

SEQ ID NO: 735 - DNA, 87 bp, Artificial Sequence
  Description of Artificial Sequence Synthetic oligonucleotide
gtttaagagctaagctggaaacagcatagcaagtttaaataaggctagtccgttgactacttgaaaaagtggcaccgagtcggtgct

SEQ ID NO: 736 - DNA, 87 bp, Artificial Sequence
  Description of Artificial Sequence Synthetic oligonucleotide
gtttaagagctaagctggaaacagcatagcaagcttaaataaggctagtccgttatcaactagaaatagtggcaccgagtcggtgct

SEQ ID NO: 737 - DNA, 87 bp, Artificial Sequence
  Description of Artificial Sequence Synthetic oligonucleotide
gtttaagagctaagctggaaacagcatagcaagtttaaataaggctaatccgttatcaacttgaaaaagtggcaccgagtcggtgct

SEQ ID NO: 738 - DNA, 87 bp, Artificial Sequence
  Description of Artificial Sequence Synthetic oligonucleotide
gtttaagagctaagctggaaacagcatagcaagttcaaataaggctagtccgttatcaacttgaaaaagtggcaccgagtcggtgct

SEQ ID NO: 739 - DNA, 87 bp, Artificial Sequence
  Description of Artificial Sequence Synthetic oligonucleotide
gtttaagagctaagctggaaacagcatagcaagtttaaataaggctagtccgttatcaagttgaaaaagtggcaccgagtcggtgct

SEQ ID NO: 740 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtccccgagtcggtgct

SEQ ID NO: 741 - DNA, 87 bp, Artificial Sequence
  Description of Artificial Sequence Synthetic oligonucleotide
gtttaagagctaagctggaaacagcatagcaagtttaaataaggcttgtccgttatcaacttgaaaaagttgcaccgtgtcggtgct

SEQ ID NO: 742 - DNA, 87 bp, Artificial Sequence
  Description of Artificial Sequence Synthetic oligonucleotide
gtttaagagctaagctggaaacagcatagcaagtttaaataaggctagtccgttatcatcctgaaaatgcggcaccgagtcggtgct

SEQ ID NO: 743 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgaaactgagtcagtttt

SEQ ID NO: 744 - DNA, 87 bp, Artificial Sequence
  Description of Artificial Sequence Synthetic oligonucleotide
gtttaagagctaagctggaaacagcatagcaagtttaaataagggtactccgttatcaacttgaaaaagtggctcagagtctgagct

SEQ ID NO: 745 - DNA, 87 bp, Artificial Sequence
  Description of Artificial Sequence Synthetic oligonucleotide
gtttaagagctaagctggaaacagcatagcaagcttaaataaggctagtccgttatcaacatgaaaatgtggcaccgagtcggtgct

SEQ ID NO: 746 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcgccgagtcggcgct

SEQ ID NO: 747 - DNA, 87 bp, Artificial Sequence
  Description of Artificial Sequence Synthetic oligonucleotide
gtttaagagctaagctggaaacagcatagcaagtttaaatatggctagtccgttatcaacttgataaagtggcaccgagtcggtgct

SEQ ID NO: 748 - DNA, 87 bp, Artificial Sequence
  Description of Artificial Sequence Synthetic oligonucleotide
atttacgagctaagctggaaacagcatagcaagtttaaataaggctagtccgttatcaacttgaaaaagtggcaccgagtcggtgct

SEQ ID NO: 749 - DNA, 87 bp, Artificial Sequence
  Description of Artificial Sequence Synthetic oligonucleotide
gtttaagagctaagctggaaacagcatagcaagtttaaataaagctagtctgttatcaactcgaaagagtgtcaccgagtcggtgat

SEQ ID NO: 750 - DNA, 87 bp, Artificial Sequence
  Description of Artificial Sequence Synthetic oligonucleotide
gtttaagagctaagctggaaacagcatagcaagattaaataaggctagtccgttatcaacttgaaaaagtggcaccgagtcggtgca

SEQ ID NO: 751 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggccaccagtggtggct

SEQ ID NO: 752 - DNA, 87 bp, Artificial Sequence
  Description of Artificial Sequence Synthetic oligonucleotide
gtttaagagctaagctggaaacagcatagcaagtttaaataaggctagtccgttatcgacttgacaaagtggcaccgagtcggtgct

SEQ ID NO: 753 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgccacggagtccgtggt

SEQ ID NO: 754 - DNA, 87 bp, Artificial Sequence
  Description of Artificial Sequence Synthetic oligonucleotide
gtttaagagctaagctggaaacagcatagcgattttaaataaggctagtccgttatcaacttgaaaaagtggcaccgagtcggtgct

SEQ ID NO: 755 - DNA, 87 bp, Artificial Sequence
  Description of Artificial Sequence Synthetic oligonucleotide
gtttaagagctaagctggaaacagcatagcaaatttaaataaggctcgtccgttatcaactttaaaaagtggcaccgagtcggtgct

SEQ ID NO: 756 - DNA, 87 bp, Artificial Sequence
  Description of Artificial Sequence Synthetic oligonucleotide
gtttaagagctaagctggaaacagcatagcaagtttaaataaggctagtctgttatcaacttgaaaaagtggcaccgagtcggtgct

SEQ ID NO: 757 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtgacaccgagtcggtgtt

SEQ ID NO: 758 - DNA, 87 bp, Artificial Sequence
  Description of Artificial Sequence Synthetic oligonucleotide
gtttaagagctaagctggaaacagcatggcaagtttaaataaggctcgtccgttatgaacttgaaaaagtggcaccgagtcggtgct

SEQ ID NO: 759 - DNA, 87 bp, Artificial Sequence
  Description of Artificial Sequence Synthetic oligonucleotide
gtttaagagctaagctggaaacagcataacaagtttaaataaggctagtccgtcataaacttgaaaaagtggcaccgagtcggtgct

SEQ ID NO: 760 - DNA, 87 bp, Artificial Sequence
  Description of Artificial Sequence Synthetic oligonucleotide
gtttaagagctaagctggaaacagcatagcaagcttaaataaggctagtccgttatcaagttgaaaaactggcaccgagtcggtgct

SEQ ID NO: 761 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggtacgcagtgcgtact

SEQ ID NO: 762 - DNA, 87 bp, Artificial Sequence
  Description of Artificial Sequence Synthetic oligonucleotide
gtttaagagctaagctggaaacagcatagcaagtttaaataaggctagtccgatataaacttgcaaaagtggcaccgagtcggtgct

SEQ ID NO: 763 - DNA, 87 bp, Artificial Sequence
  Description of Artificial Sequence Synthetic oligonucleotide
gtttaagagctaagctggaaacagcatagcaagtttaaataaggctagtccgtgattaacttgaaaaagtggcaccgagacggtgct

SEQ ID NO: 764 - DNA, 87 bp, Artificial Sequence
  Description of Artificial Sequence Synthetic oligonucleotide
gtttaagagctaagctggaaacagcatagcgagtttaaataaggctagtccgttatctacttgaaaaagtggcaccgagtcggtgct

SEQ ID NO: 765 - DNA, 87 bp, Artificial Sequence
  Description of Artificial Sequence Synthetic oligonucleotide
gtttaagagctaagctggaaacagcatagcaagtttaaataaggatattccgttatcaaattgaaaaattggctccgagtcggagct

SEQ ID NO: 766 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggctccgagtcggagct

SEQ ID NO: 767 - DNA, 87 bp, Artificial Sequence
  Description of Artificial Sequence Synthetic oligonucleotide
gtttaagagctaagctggaaacagcatagcaagtttaaataaggctagtccgttatctacttgaaaaagtggcaccgagtcggtgct

SEQ ID NO: 768 - DNA, 87 bp, Artificial Sequence
  Description of Artificial Sequence Synthetic oligonucleotide
gtttaagagctaagctggaaacagcatagcaagtgtaaataaggctagtccgttatcaacttgaaaaagtggcaccgagtcggtgct

SEQ ID NO: 769 - DNA, 87 bp, Artificial Sequence
  Description of Artificial Sequence Synthetic oligonucleotide
gtttaagagctaagctggaaacagcatagcaaatttaaataaggctagtccgttatcaacttgaaaaagtggcaccgagtcggtgcc

SEQ ID NO: 770 - DNA, 87 bp, Artificial Sequence
  Description of Artificial Sequence Synthetic oligonucleotide
gtttaagagctaagctggaaacagcatagcaagtttaaataaggctagtccgttatccacttgataaagtggcaccgagtcggtgct

SEQ ID NO: 771 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtgtcaccgagtcggtgat

SEQ ID NO: 772 - DNA, 87 bp, Artificial Sequence
  Description of Artificial Sequence Synthetic oligonucleotide
atttaggagctaagctggaaacagcatagcaagtttaaataaggctagtccgttatcaacttgaaaaagtggcaccgagtcggtgct

SEQ ID NO: 773 - DNA, 87 bp, Artificial Sequence
  Description of Artificial Sequence Synthetic oligonucleotide
gtttaagagctaagctggaaacagcatagcaagtttaaataaggctagtccgttatcatgaagaaaatgaggcaccgagtcggtgct

SEQ ID NO: 774 - DNA, 87 bp, Artificial Sequence
  Description of Artificial Sequence Synthetic oligonucleotide
gtttaagagctaagctggaaacagcatagcaagtttaaataaagctagtctgttatcaacttgaaaaagtggcaccgagtcggtgct

SEQ ID NO: 775 - DNA, 87 bp, Artificial Sequence
  Description of Artificial Sequence Synthetic oligonucleotide
gtttaagagctaagctggaaacagcatagcaattttaaataaggctagtccgttatcaacttgtaaaagtggcaccgagtcggtgct

SEQ ID NO: 776 - DNA, 87 bp, Artificial Sequence
  Description of Artificial Sequence Synthetic oligonucleotide
gtttaagagctaagctggaaacagcatagcaagtttaaataaggctagtccgttatcaagttgaaaaactggcatggagtccatgct

SEQ ID NO: 777 - DNA, 87 bp, Artificial Sequence
  Description of Artificial Sequence Synthetic oligonucleotide
ttttaagagctaagctggaaacagcatagcaagtttaaataaggctagtccgttatcaacttgaaaaagtggcaccgagtcggtgct

SEQ ID NO: 778 - DNA, 87 bp, Artificial Sequence
  Description of Artificial Sequence Synthetic oligonucleotide
gtttaagagctaagctggaaacagcatagcaagtttgaataaggctagtccgttatcaacttgaaaaagtggcaccgagtcggtgct

SEQ ID NO: 779 - DNA, 87 bp, Artificial Sequence
  Description of Artificial Sequence Synthetic oligonucleotide
gtttaagagctaagctggaaacagcatagcaagtttaaatatggctagtccgttatcaacttgagaaagtggcaccgactcggtgct

SEQ ID NO: 780 - DNA, 87 bp, Artificial Sequence
  Description of Artificial Sequence Synthetic oligonucleotide
gtttaagagctaagctggaaacagcatagcaagtttaaataaagctagtctgttatcaactggaaacagtggccccgagtcggggct

SEQ ID NO: 781 - DNA, 87 bp, Artificial Sequence
  Description of Artificial Sequence Synthetic oligonucleotide
gtttaagagctaagctggaaacagcatagcaagtttaaataaagctagtctgttatcaacttgaaaaagtggcgccgagtcggcgct

SEQ ID NO: 782 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcagccagtggctgct

SEQ ID NO: 783 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgtcacacagtgtgtgat

SEQ ID NO: 784 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcccccagtgggggct

SEQ ID NO: 785 - DNA, 87 bp, Artificial Sequence
  Description of Artificial Sequence Synthetic oligonucleotide
gtttaagagctaagctggaaacagcatagcaagtttaaataaggctagtccattatcaacttgaaaaagtggcaccgagtcggtgct

SEQ ID NO: 786 - DNA, 87 bp, Artificial Sequence
  Description of Artificial Sequence Synthetic oligonucleotide
gtttaagagctaagctggaaacagcatagcaattttaaataaggctagtccgttatcaacttgaaaaagtggcaccgagtcggtgct

SEQ ID NO: 787 - DNA, 87 bp, Artificial Sequence
  Description of Artificial Sequence Synthetic oligonucleotide
gtttaagagctaagctggaaacagcatagcaagtttaaataaggctcgtccgttataaacttaaaaaagtggcaccgagtcggtgct

SEQ ID NO: 788 - DNA, 87 bp, Artificial Sequence
  Description of Artificial Sequence Synthetic oligonucleotide
gtttaagagctaagctggaaacagcatagcaagcttaaataaggctagtccgttttcaacttgataaagtggcaccgagtcggtgct

SEQ ID NO: 789 - DNA, 87 bp, Artificial Sequence
  Description of Artificial Sequence Synthetic oligonucleotide
gtttaagagctaagctggaaacagcatagcaggtttaaataaggctagtccgttatcgacttgaaaaagtggcaccgagtcggtgct

SEQ ID NO: 790 - DNA, 87 bp, Artificial Sequence
  Description of Artificial Sequence Synthetic oligonucleotide
gtttatgcgctaagctggaaacagcatagcaagtttaaataaggctagtccgttatcaacttgaaaaagtggcaccgagtcggtgct

SEQ ID NO: 791 - DNA, 87 bp, Artificial Sequence
  Description of Artificial Sequence Synthetic oligonucleotide
gtttcagagctaagctggaaacagcatagcaagtttaaataaggctagtccgttatcaacttgaaaaagtggcaccgagtcggtgct

SEQ ID NO: 792 - DNA, 87 bp, Artificial Sequence
  Description of Artificial Sequence Synthetic oligonucleotide
gttttagagctaagctggaaacagcatagcaagtttaaataaggctagtccgttatcaacttgaaaaagtggcaccgagtcggtgct

SEQ ID NO: 793 - DNA, 87 bp, Artificial Sequence
  Description of Artificial Sequence Synthetic oligonucleotide
ttttaacagctaagctggaaacagcatagcaagtttaaataaggctagtccgttatcaacttgaaaaagtggcaccgagtcggtgct

SEQ ID NO: 794 - DNA, 87 bp, Artificial Sequence
  Description of Artificial Sequence Synthetic oligonucleotide
gtttaagagctaagctggaaacagcatagcaagtttaaataaagctagtctgttatcaacttgaaaaagtgtcaccgagtcggtgat

SEQ ID NO: 795 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagttggtgct

SEQ ID NO: 796 - DNA, 87 bp, Artificial Sequence
  Description of Artificial Sequence Synthetic oligonucleotide
gtttaagagctaagctggaaacagcatagctagtttaaataaggctagtccgatatcaacttgaaaaagtcgcaccgagtcggtgct

SEQ ID NO: 797 - DNA, 87 bp, Artificial Sequence
  Description of Artificial Sequence Synthetic oligonucleotide
ggttaagagctaagctggaaacagcatagcaagtttaaataaggctagtccgttatcaacttgaaaaagtggcaccgagtcggtgct

SEQ ID NO: 798 - DNA, 87 bp, Artificial Sequence
  Description of Artificial Sequence Synthetic oligonucleotide
gtttaagagctaagctggaaacagcatagcaagtttaaataagggtactccgttatcaagttgaaaaactggcacctagtaggtgct

SEQ ID NO: 799 - DNA, 87 bp, Artificial Sequence
  Description of Artificial Sequence Synthetic oligonucleotide
gcttaaaagctaagctggaaacagcatagcaagtttaaataaggctagtccgttatcaacttgaaaaagtggcaccgagtcggtgct

SEQ ID NO: 800 - DNA, 87 bp, Artificial Sequence
  Description of Artificial Sequence Synthetic oligonucleotide
gtttaagagctaagctggaaacagcatagcaagtttaaataaagctagtctgttatcaacttgaaaaagtggtagcgagtcgctact

SEQ ID NO: 801 - DNA, 87 bp, Artificial Sequence
  Description of Artificial Sequence Synthetic oligonucleotide
gtttaagagctaagctggaaacagcatagcaagtttatataaggctagtccgttatcaacttgaaaaagtggcaccgagtcggtgct

SEQ ID NO: 802 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccggggcggtgct

SEQ ID NO: 803 - DNA, 87 bp, Artificial Sequence
  Description of Artificial Sequence Synthetic oligonucleotide
gtttaagagctaagctggaaacagcatagcaagtttaaataaagctagtctgttatcaacttgagaaagtggcaccgagtcggtgct

SEQ ID NO: 804 - DNA, 87 bp, Artificial Sequence
  Description of Artificial Sequence Synthetic oligonucleotide
gtttaagagctaagctggaaacagcatagcaagtttaaataaggctagtccgttattaacttgaaaaagttgcaccgagtcggtgct

SEQ ID NO: 805 - DNA, 87 bp, Artificial Sequence
  Description of Artificial Sequence Synthetic oligonucleotide
gtttaagagctaagctggaaacagcatagcaagattaaataaggctagtccgttatcaacttgaaaaagtggcaccgagtcggtgct

SEQ ID NO: 806 - DNA, 87 bp, Artificial Sequence
  Description of Artificial Sequence Synthetic oligonucleotide
ctttaagagctaagctggaaacagcatagcaagtttaaataaggctagtccgttatcaacttgaaaaagtggcaccgagtcggtgct

SEQ ID NO: 807 - DNA, 87 bp, Artificial Sequence
  Description of Artificial Sequence Synthetic oligonucleotide
gtttaagagctaagctggaaacagcatagcaagtttaaaaaaggctagtccgttatcaacttgaaaaagtggcaccgagtcggtgct

SEQ ID NO: 808 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcacgcagtgcgtgct

SEQ ID NO: 809 - DNA, 87 bp, Artificial Sequence
  Description of Artificial Sequence Synthetic oligonucleotide
gtttaagagctaagctggaaacagcatagcaagtttaaataaggctagtccgttatctacttgacaaagtggcaccgagtcggtgct

SEQ ID NO: 810 - DNA, 87 bp, Artificial Sequence
  Description of Artificial Sequence Synthetic oligonucleotide
gattaagagctaagctggaaacagcatagcaagtttaaataaggctagtccgttatcaacttgaaaaagtggcaccgagtcggtgct

SEQ ID NO: 811 - DNA, 87 bp, Artificial Sequence
  Description of Artificial Sequence Synthetic oligonucleotide
ggttaagcgctaagctggaaacagcatagcaagtttaaataaggctagtccgttatcaacttgaaaaagtggcaccgagtcggtgct

SEQ ID NO: 812 - DNA, 87 bp, Artificial Sequence
  Description of Artificial Sequence Synthetic oligonucleotide
gtttaagagctaagctggaaacagcatagcaagtttaaataaggctagcccgttatgaacttgaaaaagtggcaccgagtcggtgct

SEQ ID NO: 813 - DNA, 87 bp, Artificial Sequence
  Description of Artificial Sequence Synthetic oligonucleotide
gtttaagagctaagctggaaacagcatagcaagtttaaatcaggctagtccgttatcaacttgaaaaagtggcaccgagtcggtgct

SEQ ID NO: 814 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggccccgagtcggggct

SEQ ID NO: 815 - DNA, 87 bp, Artificial Sequence
  Description of Artificial Sequence Synthetic oligonucleotide
gtttaagagctaagctggaaacagcatagcaagtttaaataaggctagtccgttgtctacttgaaaaagtggcaccgagtcggtgct

SEQ ID NO: 816 - DNA, 87 bp, Artificial Sequence
  Description of Artificial Sequence Synthetic oligonucleotide
gtctaaaagctaagctggaaacagcatagcaagtttaaataaggctagtccgttatcaacttgaaaaagtggcaccgagtcggtgct

SEQ ID NO: 817 - DNA, 87 bp, Artificial Sequence
  Description of Artificial Sequence Synthetic oligonucleotide
gtttaagagctaagctggaaacagcatagcaattttaaataaggctagtccgttatcaacttgataaagtggcaccgagtcggtgct

SEQ ID NO: 818 - DNA, 87 bp, Artificial Sequence
  Description of Artificial Sequence Synthetic oligonucleotide
gtttaagagctaagctggaaacagcatagcaagtttaaataaggatattccgttatcaacgtgaaaacgtggcacccagtgggtgct

SEQ ID NO: 819 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcaacgagtcgttgct

SEQ ID NO: 820 - DNA, 87 bp, Artificial Sequence
  Description of Artificial Sequence Synthetic oligonucleotide
gtttaagagctaagctggaaacagcatagcaagtttaaataaggctagtccattatcaacttgaataagtggcaccgagtcggtgct

SEQ ID NO: 821 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcatcgagtcgatgct

SEQ ID NO: 822 - DNA, 87 bp, Artificial Sequence
  Description of Artificial Sequence Synthetic oligonucleotide
gtttaagagctaagctggaaacagcatcgcaagtttaaataaggctagtccattatcaacttgaaaaagtggcaccgagtcggtgct

SEQ ID NO: 823 - DNA, 87 bp, Artificial Sequence
  Description of Artificial Sequence Synthetic oligonucleotide
gtttaagagctaagctggaaacagcatagcaattttaaataaggctagtccgttatcaacttgaagaagtggcaccgagtcggtgct

SEQ ID NO: 824 - DNA, 87 bp, Artificial Sequence
  Description of Artificial Sequence Synthetic oligonucleotide
gtttaagagctaagctggaaacagcatagcaagtttaaataaggctagtccattatcaacttgtaaaagtggcaccgattcggtgct

SEQ ID NO: 825 - DNA, 87 bp, Artificial Sequence
  Description of Artificial Sequence Synthetic oligonucleotide
gtttaagagctaagctggaaacagcatagcaattttaaataaggctagtccgttatcaacttgacaaagtggcaccgagtcggtgct

SEQ ID NO: 826 - DNA, 87 bp, Artificial Sequence
  Description of Artificial Sequence Synthetic oligonucleotide
gtttaagagctaagctggaaacagcattgcaagtttaaataaggctagtccattatcaacttgaaaaagtggcaccgagtcggtgct

SEQ ID NO: 827 - DNA, 87 bp, Artificial Sequence
  Description of Artificial Sequence Synthetic oligonucleotide
gtttaagagctaagctggaaacagcatagcaagtttaaaaaaggctggtccgttatcaacttgaaaaagtggcaccgagtcggtgct

SEQ ID NO: 828 - DNA, 87 bp, Artificial Sequence
  Description of Artificial Sequence Synthetic oligonucleotide
gtttaagagctaagctggaaacagcatagcaagtttaaataagtgtactacgttatcaacctgaaaaggtggcaccgagtcggtgct

SEQ ID NO: 829 - DNA, 87 bp, Artificial Sequence
  Description of Artificial Sequence Synthetic oligonucleotide
aattaagagctaagctggaaacagcatagcaagtttaaataaggctagtccgttatcaacttgaaaaagtggcaccgagtcggtgct

SEQ ID NO: 830 - DNA, 87 bp, Artificial Sequence
  Description of Artificial Sequence Synthetic oligonucleotide
gtttaagagctaagctggaaacagcatagcaagtttaaataaagctagtctgttatcaactagaaatagtggcacagagtctgtgct

SEQ ID NO: 831 - DNA, 87 bp, Artificial Sequence
  Description of Artificial Sequence Synthetic oligonucleotide
gtttaagagctaagctggaaacagcatagcaagattaaataaggctagtccgttatcaacttgataaagtggcaccgagtcggtgct

SEQ ID NO: 832 - DNA, 87 bp, Artificial Sequence
  Description of Artificial Sequence Synthetic oligonucleotide
gtttaagagctaagctggaaacagcatagcaagtttaaataaggatagtccgttatcaacttgaaaaagtggcaccgagtcggtgct

SEQ ID NO: 833 - DNA, 87 bp, Artificial Sequence
  Description of Artificial Sequence Synthetic oligonucleotide
gttttagcgctaagctggaaacagcatagcaagtttaaataaggctagtccgttatcaacttgaaaaagtggcaccgagtcggtgct

SEQ ID NO: 834 - DNA, 87 bp, Artificial Sequence
  Description of Artificial Sequence Synthetic oligonucleotide
ggttaacagctaagctggaaacagcatagcaagtttaaataaggctagtccgttatcaacttgaaaaagtggcaccgagtcggtgct

SEQ ID NO: 835 - DNA, 87 bp, Artificial Sequence
  Description of Artificial Sequence Synthetic oligonucleotide
gtttaagagctaagctggaaacagcatagcaagtttaaataagggtactccgttatcaacttgaaaaagtggcctcgagtcgaggct

SEQ ID NO: 836 - DNA, 87 bp, Artificial Sequence
  Description of Artificial Sequence Synthetic oligonucleotide
gtttaagagctaagctggaaacagcatagcgagtttaaataaggctcgtccgttatccacttgaaaaagtggcaccgagtcggtgct

SEQ ID NO: 837 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtggcattgagtcaatgct

SEQ ID NO: 838 - DNA, 87 bp, Artificial Sequence
  Description of Artificial Sequence Synthetic oligonucleotide
gtttaagagctaagctggaaacagcatagcaagtttaaataaggctagtccgttatcaaattgaaaaattgacacctagtaggtgtt

SEQ ID NO: 839 - DNA, 87 bp, Artificial Sequence
  Description of Artificial Sequence Synthetic oligonucleotide
gattaagtgctaagctggaaacagcatagcaagtttaaataaggctagtccgttatcaacttgaaaaagtggcaccgagtcggtgct

SEQ ID NO: 840 - DNA, 87 bp, Artificial Sequence
  Description of Artificial Sequence Synthetic oligonucleotide
gtttaagagctaagctggaaacagcatagcaagtataaataaggctagtccgttatcaacttgaaaaagtggcaccgagtcggtgct

SEQ ID NO: 841 - DNA, 87 bp, Artificial Sequence
  Description of Artificial Sequence Synthetic oligonucleotide
gtttaagagctaagctggaaacagcatagcaagcttaaataaggttaatccgttatcaacttgaaaaagtggcaccgagtcggtgct

SEQ ID NO: 842 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtgccaccgagtcggtggt

SEQ ID NO: 843 - DNA, 87 bp, Artificial Sequence
  Description of Artificial Sequence Synthetic oligonucleotide
gtttaagagctaagctggaaacagcatagcaagtttaaataaagttaatctgttatcaactcgaaagagtggcaccgagtcggtgct

SEQ ID NO: 844 - DNA, 87 bp, Artificial Sequence
  Description of Artificial Sequence Synthetic oligonucleotide
ctttaaaagctaagctggaaacagcatagcaagtttaaataaggctagtccgttatcaacttgaaaaagtggcaccgagtcggtgct

SEQ ID NO: 845 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcacagagtctgtgct

SEQ ID NO: 846 - DNA, 87 bp, Artificial Sequence
  Description of Artificial Sequence Synthetic oligonucleotide
gtttaagagctaagctggaaacagcatagcaattttaaataaggctagtccgttatcaacttgaaaaagtggcaccgaatcggtgct

SEQ ID NO: 847 - DNA, 87 bp, Artificial Sequence
  Description of Artificial Sequence Synthetic oligonucleotide
gtttaagagctaagctggaaacagcatagcaagcttaaataaggctagtccgttatcatcttgaaaaagaggcaccgagtcggtgct

SEQ ID NO: 848 - DNA, 87 bp, Artificial Sequence
  Description of Artificial Sequence Synthetic oligonucleotide
gtttaagagctaagctggaaacagcatagcaagtttaaataaagctagtctgttatcatcttgaaaaagaggctccgagtcggagct

SEQ ID NO: 849 - DNA, 87 bp, Artificial Sequence
  Description of Artificial Sequence Synthetic oligonucleotide
gtttaagagctaagctggaaacagcatagcaagtttaaatcaggctagtccataatcaacttgaaaaagtggcaccgagtcggtgct

SEQ ID NO: 850 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtgggtgct

SEQ ID NO: 851 - DNA, 87 bp, Artificial Sequence
  Description of Artificial Sequence Synthetic oligonucleotide
gtttaagagctaagctggaaacagcatagcaagtttaaataaggatattccgttatcagcttgaaaaagcggcacccagtgggtgct

SEQ ID NO: 852 - DNA, 87 bp, Artificial Sequence
  Description of Artificial Sequence Synthetic oligonucleotide
gtttaagagctaagctggaaacagcatagcaattttaaataaggcgagtccgttatcaacttgaaaaagtggcaccgagtcggtgct

SEQ ID NO: 853 - DNA, 87 bp, Artificial Sequence
  Description of Artificial Sequence Synthetic oligonucleotide
gtttaagagctaagctggaaacagcatagcaagtttaaataaggctagtacgttatcaacttgaaaaagtggcaccgagtcggtgct

SEQ ID NO: 854 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcagggagtccctgct

SEQ ID NO: 855 - DNA, 87 bp, Artificial Sequence
  Description of Artificial Sequence Synthetic oligonucleotide
gttcaagagctaagctggaaacagcatagcaagtttaaataaggctagtccgttatcaacttgaaaaagtggcaccgagtcggtgct

SEQ ID NO: 856 - DNA, 87 bp, Artificial Sequence
  Description of Artificial Sequence Synthetic oligonucleotide
gtttaagagctaagctggaaacagcatagcaagtttaaatcaggctagtccgttatcaacttgaaaaagtggcaccgagccggtgct

SEQ ID NO: 857 - DNA, 87 bp, Artificial Sequence
  Description of Artificial Sequence Synthetic oligonucleotide
gtttaagagctaagctggaaacagcatagcaattttaaataaggctagtccgttatcaacttaaacaagtggcaccgagtcggtgct

SEQ ID NO: 858 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcagcgagtcgctgct

SEQ ID NO: 859 - DNA, 87 bp, Artificial Sequence
  Description of Artificial Sequence Synthetic oligonucleotide
gtttaagagctaagctggaaacagcatagcaagtttaaataagaatatttcgttatcaacttgaaaaagtggcaacgagtcgttgct

SEQ ID NO: 860 - DNA, 87 bp, Artificial Sequence
  Description of Artificial Sequence Synthetic oligonucleotide
gtttaagagctaagctggaaacagcatagcaagtttaaatagggctagtccggtatcaacttgaaaaagtcgcaccgagtcggtgct

SEQ ID NO: 861 - DNA, 87 bp, Artificial Sequence
  Description of Artificial Sequence Synthetic oligonucleotide
gtttaagagctaagctggaaacagcatagcaagcttaaataagcctagtgcgttatcaacttgaaaaagtggcaccgagtcggtgct

SEQ ID NO: 862 - DNA, 87 bp, Artificial Sequence
  Description of Artificial Sequence Synthetic oligonucleotide
gtttaagagctaagctggaaacagcatagcaggtttaaatcaggctagtccgttatcaacttgataaagtggcaccgagtcggtgct

SEQ ID NO: 863 - DNA, 87 bp, Artificial Sequence
  Description of Artificial Sequence Synthetic oligonucleotide
gtttaagagctaagctggaaacagcatagcaagtttaaataagaatatttcgttatcaacttgaaaaagtggcaccgagtcggtgct

SEQ ID NO: 864 - DNA, 87 bp, Artificial Sequence
  Description of Artificial Sequence Synthetic oligonucleotide
gttttatagctaagctggaaacagcatagcaagtttaaataaggctagtccgttatcaacttgaaaaagtggcaccgagtcggtgct

SEQ ID NO: 865 - DNA, 87 bp, Artificial Sequence
  Description of Artificial Sequence Synthetic oligonucleotide
gtttaagagctaagctggaaacagcatagcaagttgaaataaggctagtccgttatcaacttgaaaaagtggcaccgagtcggtgct

SEQ ID NO: 866 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtggcacccagtgggtgct

SEQ ID NO: 867 - DNA, 87 bp, Artificial Sequence
  Description of Artificial Sequence Synthetic oligonucleotide
gtttaagagctaagctggaaacagcatagcaaatttaaataaggctagtccgttataaacttgaaaaagtggcaccgagtcggtgct

SEQ ID NO: 868 - DNA, 87 bp, Artificial Sequence
  Description of Artificial Sequence Synthetic oligonucleotide
gtttaagagctaagctggaaacagcatagcaagtttaaataaggcttgtccgttatatacttgaaaaagtggcaccgagtcggtgct

SEQ ID NO: 869 - DNA, 87 bp, Artificial Sequence
  Description of Artificial Sequence Synthetic oligonucleotide
gtttaagagctaagctggaaacagcatagcaagtttaaataaggctactccgttatcaacttgaaaaagtggcaccgagtcggtgct

SEQ ID NO: 870 - DNA, 87 bp, Artificial Sequence
  Description of Artificial Sequence Synthetic oligonucleotide
gtttaagagctaagctggaaacagcatagcaagtttaaataaggctagtccggtatctacttgaaaaagtggcaccgagtcggtgct

SEQ ID NO: 871 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtaggtgct

SEQ ID NO: 872 - DNA, 87 bp, Artificial Sequence
  Description of Artificial Sequence Synthetic oligonucleotide
gttttagtgctaagctggaaacagcatagcaagtttaaataaggctagtccgttatcaacttgaaaaagtggcaccgagtcggtgct

SEQ ID NO: 873 - DNA, 87 bp, Artificial Sequence
  Description of Artificial Sequence Synthetic oligonucleotide
gtttcagggctaagctggaaacagcatagcaagtttaaataaggctagtccgttatcaacttgaaaaagtggcaccgagtcggtgct

SEQ ID NO: 874 - DNA, 87 bp, Artificial Sequence
  Description of Artificial Sequence Synthetic oligonucleotide
gtttaagagctaagctggaaacagcatagcaattttaaataaggctagcccgttttcaacttgaaaaagtggcaccgagtcggtgct

SEQ ID NO: 875 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcaccaagttggtgct

SEQ ID NO: 876 - DNA, 87 bp, Artificial Sequence
  Description of Artificial Sequence Synthetic oligonucleotide
gtttaagagctaagctggaaacagcatagcaactttaaataaggctagtccgttatcaactcgaaagagtggcaccgagtcggtgct

SEQ ID NO: 877 - DNA, 87 bp, Artificial Sequence
  Description of Artificial Sequence Synthetic oligonucleotide
gtttaagagctaagctggaaacagcataacaagtttaaataaggctagtccattatcaacttgaacaagtggcaccgagtcggtgct

SEQ ID NO: 878 - DNA, 87 bp, Artificial Sequence
  Description of Artificial Sequence Synthetic oligonucleotide
gtttaagagctaagctggaaacagcatagcaagtttaagtaaggctagtccgttatcaacttgaaaaagtggcaccgagtcggtgct

SEQ ID NO: 879 - DNA, 87 bp, Artificial Sequence
  Description of Artificial Sequence Synthetic oligonucleotide
gtttaagagctaagctggaaacagcatagcaagtttaaataaggctagttcgttatgaacttgaaaaagtggcaccgagtcggtgct

SEQ ID NO: 880 - DNA, 87 bp, Artificial Sequence
  Description of Artificial Sequence Synthetic oligonucleotide
attttagagctaagctggaaacagcatagcaagtttaaataaggctagtccgttatcaacttgaaaaagtggcaccgagtcggtgct

SEQ ID NO: 881 - DNA, 87 bp, Artificial Sequence
  Description of Artificial Sequence Synthetic oligonucleotide
gtttaagagctaagctggaaacagcatagcaaatttaaataaggctagtccgttatgaacttgagaaagtggcaccgagtcggtgct

SEQ ID NO: 882 - DNA, 87 bp, Artificial Sequence
  Description of Artificial Sequence Synthetic oligonucleotide
gtttcatagctaagctggaaacagcatagcaagtttaaataaggctagtccgttatcaacttgaaaaagtggcaccgagtcggtgct

SEQ ID NO: 883 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaccgagtcgctgct

SEQ ID NO: 884 - DNA, 87 bp, Artificial Sequence
  Description of Artificial Sequence Synthetic oligonucleotide
gtttaagagctaagctggaaacagcatagcaagtttaaatacggctagtccgttatcaacttgaaaaagtggcaccgagtcggtgct

SEQ ID NO: 885 - DNA, 87 bp, Artificial Sequence
  Description of Artificial Sequence Synthetic oligonucleotide
gtttaagagctaagctggaaacagcatagcaagtttaaataagggtagtccgttatcaacttgaaaaagtggcaccgagtcggtgct

SEQ ID NO: 886 - DNA, 87 bp, Artificial Sequence
  Description of Artificial Sequence Synthetic oligonucleotide
gtttaagagctaagctggaaacagcatagcaagtttaaataaagctagtctgttatcaaattgaaaaattgtcaccgagtcggtgat

SEQ ID NO: 887 - DNA, 87 bp, Artificial Sequence
  Description of Artificial Sequence Synthetic oligonucleotide
gtttaagagctaagctggaaacagcatagcaagtttaaatatggctagtccgttatcaacttgaaaaagtagcaccgaatcggtgct

SEQ ID NO: 888 - DNA, 87 bp, Artificial Sequence
  Description of Artificial Sequence Synthetic oligonucleotide
gtttaagagctaagctggaaacagcatagctagtttaaataaggctagtccgttgtctacttgaaaaagtggcaccgagtcggtgct

SEQ ID NO: 889 - DNA, 87 bp, Artificial Sequence
  Description of Artificial Sequence Synthetic oligonucleotide
gtttaagagctaagctggaaacagcatagcaagtttaaataaggctagtcctttaacaacttgaaaaagtggcaccgagtcggtgct

SEQ ID NO: 890 - DNA, 87 bp, Artificial Sequence
  Description of Artificial Sequence Synthetic oligonucleotide
gttttacagctaagctggaaacagcatagcaagtttaaataaggctagtccgttatcaacttgaaaaagtggcaccgagtcggtgct

SEQ ID NO: 891 - DNA, 87 bp, Artificial Sequence
  Description of Artificial Sequence Synthetic oligonucleotide
gtttaagagctaagctggaaacagcatagcaagtttaaataaggatattccgttatcaacttgaaaaagtggcatccagtggatgct

SEQ ID NO: 892 - DNA, 87 bp, Artificial Sequence
  Description of Artificial Sequence Synthetic oligonucleotide
gtttaagagctaagctggaaacagcatagcaaatttaaataaggctagtccgttatcaacttgaaaaagtagcaccgagtcggtgct

SEQ ID NO: 893 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcactgagtcagtgct

SEQ ID NO: 894 - DNA, 87 bp, Artificial Sequence
  Description of Artificial Sequence Synthetic oligonucleotide
gtttaagagctaagctggaaacagcatagcaagtttaaataaggctattccgttatcaacttgaaaaagtggcaccgagtcggtgct

SEQ ID NO: 895 - DNA, 87 bp, Artificial Sequence
  Description of Artificial Sequence Synthetic oligonucleotide
gtttaagagctaagctggaaacagcatagcaagtttaaataaggctagtccgttatcaaattgaaaaattggcgcccagtgggcgct

SEQ ID NO: 896 - DNA, 87 bp, Artificial Sequence
  Description of Artificial Sequence Synthetic oligonucleotide
gtttaagagctaagctggaaacagcatagcaactttaaataaggctagtccgttatcaacctgaaaaggtggcaccgagtcggtgct

SEQ ID NO: 897 - DNA, 87 bp, Artificial Sequence
  Description of Artificial Sequence Synthetic oligonucleotide
gtttaagagctaagctggaaacagcatagcaagtttaaataaggctagtccgttatgtacttcaaaaagtggcaccgagtcggtgct

SEQ ID NO: 898 - DNA, 87 bp, Artificial Sequence
  Description of Artificial Sequence Synthetic oligonucleotide
gtttataagctaagctggaaacagcatagcaagtttaaataaggctagtccgttatcaacttgaaaaagtggcaccgagtcggtgct

SEQ ID NO: 899 - DNA, 87 bp, Artificial Sequence
  Description of Artificial Sequence Synthetic oligonucleotide
gtttaagagctaagctggaaacagcatagcaagtttaaataaggctagtccttaatcaacttgaagaagtggcaccgagtcggtgct

SEQ ID NO: 900 - DNA, 87 bp, Artificial Sequence
  Description of Artificial Sequence Synthetic oligonucleotide
gtttaagagctaagctggaaacagcatagcaagtttaaatcaggctagtccgttatcaacttgaaaaagttgcaccgagtcggtgct

SEQ ID NO: 901 - DNA, 87 bp, Artificial Sequence
  Description of Artificial Sequence Synthetic oligonucleotide
ggttaaaagctaagctggaaacagcatagcaagtttaaataaggctagtccgttatcaacttgaaaaagtggcaccgagtcggtgct

SEQ ID NO: 902 - DNA, 87 bp, Artificial Sequence
  Description of Artificial Sequence Synthetic oligonucleotide
gtttaagagctaagctggaaacagcatagcaagcttaaataagactagttcgttatcaacttgaaaaagtggcaccgagtcggtgct

SEQ ID NO: 903 - DNA, 87 bp, Artificial Sequence
  Description of Artificial Sequence Synthetic oligonucleotide
gtttaagagctaagctggaaacagcatagcaagtttaaataagactagtccgttatcaacttgaaaaagtggcaccgagtcggtgct

SEQ ID NO: 904 - DNA, 87 bp, Artificial Sequence
  Description of Artificial Sequence Synthetic oligonucleotide
gtttaagagctaagctggaaacagcatagcaagtttagataaggctagtccgttatcaacttgaaaaagtggcaccgagtcggtgct

SEQ ID NO: 905 - DNA, 87 bp, Artificial Sequence
  Description of Artificial Sequence Synthetic oligonucleotide
ctttgagagctaagctggaaacagcatagcaagtttaaataaggctagtccgttatcaacttgaaaaagtggcaccgagtcggtgct

SEQ ID NO: 906 - DNA, 87 bp, Artificial Sequence
  Description of Artificial Sequence Synthetic oligonucleotide
gtttaagagctaagctggaaacagcatagcaagtttaaataaggctagtccattatcaacttgaaaaagttgcaccgagtcggtgct

SEQ ID NO: 907 - DNA, 87 bp, Artificial Sequence
  Description of Artificial Sequence Synthetic oligonucleotide
gtttaagagctaagctggaaacagcatagcaactttaaataaggctagtccgttatcaactggaaacagtggcaccgagtcggtgct

SEQ ID NO: 908 - DNA, 87 bp, Artificial Sequence
  Description of Artificial Sequence Synthetic oligonucleotide
gttcaagtgctaagctggaaacagcatagcaagtttaaataaggctagtccgttatcaacttgaaaaagtggcaccgagtcggtgct

SEQ ID NO: 909 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctccgagtctgcgct

SEQ ID NO: 910 - DNA, 87 bp, Artificial Sequence
  Description of Artificial Sequence Synthetic oligonucleotide
gtttaagagctaagctggaaacagcatagcaagttaaaataaggctagtccgttatcaacttgaaaaagtggcaccgagtcggtgct

SEQ ID NO: 911 - DNA, 87 bp, Artificial Sequence
  Description of Artificial Sequence Synthetic oligonucleotide
gtttaagagctaagctggaaacagcatagcaagtttaactaaggctagtccgttatcaacttgaaaaagtggcaccgagtcggtgct

SEQ ID NO: 912 - DNA, 87 bp, Artificial Sequence
  Description of Artificial Sequence Synthetic oligonucleotide
gtttaagagctaagctggaaacagcatagcaactttaaataaggctagtccgttatcaccttgaaaaaggggcaccgagtcggtgct

SEQ ID NO: 913 - DNA, 87 bp, Artificial Sequence
  Description of Artificial Sequence Synthetic oligonucleotide
gtctgagagctaagctggaaacagcatagcaagtttaaataaggctagtccgttatcaacttgaaaaagtggcaccgagtcggtgct

SEQ ID NO: 914 - DNA, 87 bp, Artificial Sequence
  Description of Artificial Sequence Synthetic oligonucleotide
gtttaagagctaagctggaaacagcatagcaagtttaaataaagttaatctgttatcaatttgaaaaaatggcaccgagtcggtgct

SEQ ID NO: 915 - DNA, 87 bp, Artificial Sequence
  Description of Artificial Sequence Synthetic oligonucleotide
gtttaagagctaagctggaaacagcatagcaagcttaaataaggctagtccgttatcaaattgaaaaattggcaccgagtcggtgct

SEQ ID NO: 916 - DNA, 87 bp, Artificial Sequence
  Description of Artificial Sequence Synthetic oligonucleotide
gtttaagagctaagctggaaacagcatagcaactttaaataaggctagtccgttatcagcttgaaaaagcggcaccgagtcggtgct

SEQ ID NO: 917 - DNA, 87 bp, Artificial Sequence
  Description of Artificial Sequence Synthetic oligonucleotide
attcaagagctaagctggaaacagcatagcaagtttaaataaggctagtccgttatcaacttgaaaaagtggcaccgagtcggtgct

SEQ ID NO: 918 - DNA, 87 bp, Artificial Sequence
  Description of Artificial Sequence Synthetic oligonucleotide
gtttaagagctaagctggaaacagcatagcacgtttaaataaggctagtcctttatcaacttgaaaaagtggcaccgagtcggtgct

SEQ ID NO: 919 - DNA, 87 bp, Artificial Sequence
  Description of Artificial Sequence Synthetic oligonucleotide
gtttaagagctaagctggaaacagcatagcaactttaaataaggctagtccgttatcaacgtgaaaacgtggcaccgagtcggtgct

SEQ ID NO: 920 - DNA, 87 bp, Artificial Sequence
  Description of Artificial Sequence Synthetic oligonucleotide
gtttaagagctaagctggaaacagcatagcaagtttaaataaagctagtctgttatcaacttgaaaaagtggcacccagtgggtgct

SEQ ID NO: 921 - DNA, 87 bp, Artificial Sequence
  Description of Artificial Sequence Synthetic oligonucleotide
gtttaagagctaagctggaaacagcatagcaagtttaaataaggctagtcctttatcaacttgaaaaagtggcaccgagtcggtgct

SEQ ID NO: 922 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggtaccgagtcggtact

SEQ ID NO: 923 - DNA, 87 bp, Artificial Sequence
  Description of Artificial Sequence Synthetic oligonucleotide
cgttaagagctaagctggaaacagcatagcaagtttaaataaggctagtccgttatcaacttgaaaaagtggcaccgagtcggtgct

SEQ ID NO: 924 - DNA, 87 bp, Artificial Sequence
  Description of Artificial Sequence Synthetic oligonucleotide
gtttgcgagctaagctggaaacagcatagcaagtttaaataaggctagtccgttatcaacttgaaaaagtggcaccgagtcggtgct

SEQ ID NO: 925 - DNA, 87 bp, Artificial Sequence
  Description of Artificial Sequence Synthetic oligonucleotide
gtttaagagctaagctggaaacagcatagcaagtttaaatacggcttgtccgttatcaacttcaaaaagtggcaccgagtcggtgct

SEQ ID NO: 926 - DNA, 87 bp, Artificial Sequence
  Description of Artificial Sequence Synthetic oligonucleotide
gtttaagagctaagctggaaacagcatagccagtttaaatacggctagtccgttgtcaacttgaaaaagtggcaccgagtcggtgct

SEQ ID NO: 927 - DNA, 87 bp, Artificial Sequence
  Description of Artificial Sequence Synthetic oligonucleotide
gcttacgagctaagctggaaacagcatagcaagtttaaataaggctagtccgttatcaacttgaaaaagtggcaccgagtcggtgct

SEQ ID NO: 928 - DNA, 87 bp, Artificial Sequence
  Description of Artificial Sequence Synthetic oligonucleotide
gtttaagagctaagctggaaacagcatagcaagcttaaataaggctagtccgttatcaatttgaaaaaatggcaccgagtcggtgct

SEQ ID NO: 929 - DNA, 87 bp, Artificial Sequence
  Description of Artificial Sequence Synthetic oligonucleotide
gtttaagagctaagctggaaacagcatagcaagtttaaataatgctagtcagttatcaactggaaacagtggcaccgagtcggtgct

SEQ ID NO: 930 - DNA, 87 bp, Artificial Sequence
  Description of Artificial Sequence Synthetic oligonucleotide
gtttaagagctaagctggaaacagcatggcaagtttaaatacggctagaccgttatcaacttgaaaaagtggcaccgagtcggtgct

SEQ ID NO: 931 - DNA, 87 bp, Artificial Sequence
  Description of Artificial Sequence Synthetic oligonucleotide
gtttaagagctaagctggaaacagcatagcaagtttaaataaggctagtcctttatcaacttgaaaaagtggcaccgactcggtgct

SEQ ID NO: 932 - DNA, 87 bp, Artificial Sequence
  Description of Artificial Sequence Synthetic oligonucleotide
gtttaagagctaagctggaaacagcatagcaagtttaaataaagctagtctgttatcaatttgaaaaaatgccaccgagtcggtggt

SEQ ID NO: 933 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtgggaccgagtcggtcct

SEQ ID NO: 934 - DNA, 87 bp, Artificial Sequence
  Description of Artificial Sequence Synthetic oligonucleotide
gttcaacagctaagctggaaacagcatagcaagtttaaataaggctagtccgttatcaacttgaaaaagtggcaccgagtcggtgct

SEQ ID NO: 935 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggaaccgagtcggttct

SEQ ID NO: 936 - DNA, 87 bp, Artificial Sequence
  Description of Artificial Sequence Synthetic oligonucleotide
gtttaagagctaagctggaaacagcatagctagtttaaatgaggctagtccgtaatcaacttgaaaaagtggcaccgagtcggtgct

SEQ ID NO: 937 - DNA, 87 bp, Artificial Sequence
  Description of Artificial Sequence Synthetic oligonucleotide
gtttaagagctaagctggaaacagcatagcaagtttaaataatgctagtcagttatcaactcgaaagagtggcaccgagtcggtgct

SEQ ID NO: 938 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggtaccgagtcgttcct

SEQ ID NO: 939 - DNA, 87 bp, Artificial Sequence
  Description of Artificial Sequence Synthetic oligonucleotide
gtttaagagctaagctggaaacagcatagcaagcttaaataaggatattccgttatcaacttgaaaaagtggcaccgagtcggtgct

SEQ ID NO: 940 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcaccgagtcggtgct

SEQ ID NO: 941 - DNA, 87 bp, Artificial Sequence
  Description of Artificial Sequence Synthetic oligonucleotide
ttttatgagctaagctggaaacagcatagcaagtttaaataaggctagtccgttatcaacttgaaaaagtggcaccgagtcggtgct

SEQ ID NO: 942 - DNA, 87 bp, Artificial Sequence
  Description of Artificial Sequence Synthetic oligonucleotide
ctttaggagctaagctggaaacagcatagcaagtttaaataaggctagtccgttatcaacttgaaaaagtggcaccgagtcggtgct

SEQ ID NO: 943 - DNA, 87 bp, Artificial Sequence
  Description of Artificial Sequence Synthetic oligonucleotide
gtttaagagctaagctggaaacagcatagcaagtttaaattaggctagtccgttatcaacttgaaaaagtggcaccgagtcggtgct

SEQ ID NO: 944 - DNA, 87 bp, Artificial Sequence
  Description of Artificial Sequence Synthetic oligonucleotide
gtttaagagctaagctggaaacagcatagcaagtttaaataatgctagtcagttatcaacctgaaaaggtggcaccgagtcggtgct

SEQ ID NO: 945 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcacctagtaggtgct

SEQ ID NO: 946 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcacggagtccgtgct

SEQ ID NO: 947 - DNA, 87 bp, Artificial Sequence
  Description of Artificial Sequence Synthetic oligonucleotide
gtttaagagctaagctggaaacagcatagcaagtttaaataagtctagtccgttatcaacttgaaaaagtggcaccgagtcggtgct

SEQ ID NO: 948 - DNA, 87 bp, Artificial Sequence
  Description of Artificial Sequence Synthetic oligonucleotide
gtttaagagctaagctggaaacagcatagcaagtttaaataatgctagtcagttatcaacttgaaaaagtggcaccgagtcggtgct

SEQ ID NO: 949 - DNA, 87 bp, Artificial Sequence
  Description of Artificial Sequence Synthetic oligonucleotide
gtttaagagctaagctggaaacagcatagcaaggttaaataaggctagtccgttatcagcttgaaaaagcggcaccgagtcggtgct

SEQ ID NO: 950 - DNA, 87 bp, Artificial Sequence
  Description of Artificial Sequence Synthetic oligonucleotide
gtttaagagctaagctggaaacagcatagcaagtttaaataatgctagtcagttatcaacttgaaaaagtggcaacgagtcgttgct

SEQ ID NO: 951 - DNA, 87 bp, Artificial Sequence
  Description of Artificial Sequence Synthetic oligonucleotide
gtttaagagctaagctggaaacagcatagcaagattaaataaggctagtccgttatcaacttgaaaaagtggcaccgtgtcggtgct

SEQ ID NO: 952 - DNA, 87 bp, Artificial Sequence
  Description of Artificial Sequence Synthetic oligonucleotide
gtttaagagctaagctggaaacagcatagcaagtttaaataaggctagtccttcatcaacttgaagaagtggcaccgagtcggtgct

SEQ ID NO: 953 - DNA, 87 bp, Artificial Sequence
  Description of Artificial Sequence Synthetic oligonucleotide
gtttaagagctaagctggaaacagcatagcaagtttaattaaggctagtccgttatcaacttgaaaaagtggcaccgagtcggtgct

SEQ ID NO: 954 - DNA, 87 bp, Artificial Sequence
  Description of Artificial Sequence Synthetic oligonucleotide
gtttaagagctaagctggaaacagcatagcaagtttaaataaggctactccgttattaacttgaaaaagtggcaccgagtcggtgct

SEQ ID NO: 955 - DNA, 87 bp, Artificial Sequence
  Description of Artificial Sequence Synthetic oligonucleotide
gttttggagctaagctggaaacagcatagcaagtttaaataaggctagtccgttatcaacttgaaaaagtggcaccgagtcggtgct

SEQ ID NO: 956 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcgccgagtcggcgct

SEQ ID NO: 957 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtgacaccgagtcggtgtt

SEQ ID NO: 958 - DNA, 87 bp, Artificial Sequence
  Description of Artificial Sequence Synthetic oligonucleotide
gtttaagagctaagctggaaacagcatagcaagtttaaataatgctagtcagttatcaacttgaaaaagtggcgccgagtcggcgct

SEQ ID NO: 959 - DNA, 87 bp, Artificial Sequence
  Description of Artificial Sequence Synthetic oligonucleotide
gtttaagagctaagctggaaacagcatagcatgtttaaattaggctagtccgttatcaacttgaaaaagtggcaccgagtcggtgct

SEQ ID NO: 960 - DNA, 87 bp, Artificial Sequence
  Description of Artificial Sequence Synthetic oligonucleotide
gtttaagagctaagctggaaacagcatagcaactttaaataaggctagtccgttatcaacatgaaaatgtggcaccgagtcggtgct

SEQ ID NO: 961 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggaaccgagtcggttct

SEQ ID NO: 962 - DNA, 87 bp, Artificial Sequence
  Description of Artificial Sequence Synthetic oligonucleotide
gtataagagctaagctggaaacagcatagcaagtttaaataaggctagtccgttatcaacttgaaaaagtggcaccgagtcggtgct

SEQ ID NO: 963 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggctccgagtcggagct

SEQ ID NO: 964 - DNA, 87 bp, Artificial Sequence
  Description of Artificial Sequence Synthetic oligonucleotide
gtttaagagctaagctggaaacagcatagcaagtttcaataaggctagtccgttatcaacttgaaaaagtggcaccgagtcggtgct

SEQ ID NO: 965 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgggtgggagtcgctcct

SEQ ID NO: 966 - DNA, 87 bp, Artificial Sequence
  Description of Artificial Sequence Synthetic oligonucleotide
gtttaagagctaagctggaaacagcatagcaaggttaaataaggctagtccgttatcaactcgaaagagtggcaccgagtcggtgct

SEQ ID NO: 967 - DNA, 87 bp, Artificial Sequence
  Description of Artificial Sequence Synthetic oligonucleotide
gtttaagagctaagctggaaacagcatagcaactttaaataaggctagtccggtatcaacttgaaaaagtggcaccgagtcggtgct

SEQ ID NO: 968 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcatcgagtcgatgct

SEQ ID NO: 969 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcaccgattcggtgct

SEQ ID NO: 970 - DNA, 87 bp, Artificial Sequence
  Description of Artificial Sequence Synthetic oligonucleotide
gtttaagagctaagctggaaacagcatagcaactttaaataaggctagtccgttatcaagttgaaaaactggcaccgagtcggtgct

SEQ ID NO: 971 - DNA, 87 bp, Artificial Sequence
  Description of Artificial Sequence Synthetic oligonucleotide
gtttaagagctaagctggaaacagcatagccattttaaataaggctagtccgttatcaacttgaaaaagtggcaccgagtcggtgct

SEQ ID NO: 972 - DNA, 87 bp, Artificial Sequence
  Description of Artificial Sequence Synthetic oligonucleotide
gtttaagagctaagctggaaacagcatagcaaggttaaataaggctagtccgttatcaacgtgaaaacgtggcaccgagtcggtgct

SEQ ID NO: 973 - DNA, 87 bp, Artificial Sequence
  Description of Artificial Sequence Synthetic oligonucleotide
gtttaagagctaagctggaaacagcatagcaagcttaaataagggtactccgttatcaacttgaaaaagtggcaccgagtcggtgct

SEQ ID NO: 974 - DNA, 87 bp, Artificial Sequence
  Description of Artificial Sequence Synthetic oligonucleotide
gttaaagagctaagctggaaacagcatagcaagtttaaataaggctagtccgttatcaacttgaaaaagtggcaccgagtcggtgct

SEQ ID NO: 975 - DNA, 87 bp, Artificial Sequence
  Description of Artificial Sequence Synthetic oligonucleotide
gtataagtgctaagctggaaacagcatagcaagtttaaataaggctagtccgttatcaacttgaaaaagtggcaccgagtcggtgct

SEQ ID NO: 976 - DNA, 87 bp, Artificial Sequence
  Description of Artificial Sequence Synthetic oligonucleotide
gtttaagagctaagctggaaacagcatagcaagtttaaataaggctaggccgttatcaacttgaaaaagtgtcaccgagtcggtgct

SEQ ID NO: 977 - DNA, 87 bp, Artificial Sequence
  Description of Artificial Sequence Synthetic oligonucleotide
gtttaagagctaagctggaaacagcatagcaagtttaaatgaggctagtccgttatcaacttgaaaaagtggcaccgagtcggtgct

SEQ ID NO: 978 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtgtcaccgagtcggtgat

SEQ ID NO: 979 - DNA, 87 bp, Artificial Sequence
  Description of Artificial Sequence Synthetic oligonucleotide
gtttaagagctaagctggaaacagcatagcaactttaaataaggctagtccgttatcaactagaaatagtggcaccgagtcggtgct

SEQ ID NO: 980 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggctccgagtcggagct

SEQ ID NO: 981 - DNA, 87 bp, Artificial Sequence
  Description of Artificial Sequence Synthetic oligonucleotide
gtgtaagagctaagctggaaacagcatagcaagtttaaataaggctagtccgttatcaacttgaaaaagtggcaccgagtcggtgct

SEQ ID NO: 982 - DNA, 87 bp, Artificial Sequence
  Description of Artificial Sequence Synthetic oligonucleotide
gttgaagagctaagctggaaacagcatagcaagtttaaataaggctagtccgttatcaacttgaaaaagtggcaccgagtcggtgct

SEQ ID NO: 983 - DNA, 87 bp, Artificial Sequence
  Description of Artificial Sequence Synthetic oligonucleotide
gtttgtgagctaagctggaaacagcatagcaagtttaaataaggctagtccgttatcaacttgaaaaagtggcaccgagtcggtgct

SEQ ID NO: 984 - DNA, 87 bp, Artificial Sequence
  Description of Artificial Sequence Synthetic oligonucleotide
gtttaagagctaagctggaaacagcatagcaagtttaaataatgctagtcagttatcaacttgagaaagtggcaccgagtcggtgct

SEQ ID NO: 985 - DNA, 87 bp, Artificial Sequence
  Description of Artificial Sequence Synthetic oligonucleotide
tttcaagagctaagctggaaacagcatagcaagtttaaataaggctagtccgttatcaacttgaaaaagtggcaccgagtcggtgct

SEQ ID NO: 986 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcaccgagtcggtgct

SEQ ID NO: 987 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtgccaccgagtcggtggt

SEQ ID NO: 988 - DNA, 87 bp, Artificial Sequence
  Description of Artificial Sequence Synthetic oligonucleotide
gtttttgagctaagctggaaacagcatagcaagtttaaataaggctagtccgttatcaacttgaaaaagtggcaccgagtcggtgct

SEQ ID NO: 989 - DNA, 87 bp, Artificial Sequence
  Description of Artificial Sequence Synthetic oligonucleotide
gattgagagctaagctggaaacagcatagcaagtttaaataaggctagtccgttatcaacttgaaaaagtggcaccgagtcggtgct

SEQ ID NO: 990 - DNA, 87 bp, Artificial Sequence
  Description of Artificial Sequence Synthetic oligonucleotide
gtttaagagctaagctggaaacagcatagcaagtttaaataaagctagtccgttatcaacttgaaaaagtggcaccgagtcggtgct

SEQ ID NO: 991 - DNA, 87 bp, Artificial Sequence
  Description of Artificial Sequence Synthetic oligonucleotide
gtttaagagctaagctggaaacagcatagctagtttaaataaggctaggccgtaatcaacttgaaaaagtggcaccgagtcggtgct

SEQ ID NO: 992 - DNA, 87 bp, Artificial Sequence
  Description of Artificial Sequence Synthetic oligonucleotide
gtttaagagctaagctggaaacagcatagcaagtttaaataatgctagtcagttatcaccttgaaaaaggggtaccgagtcggtact

SEQ ID NO: 993 - DNA, 87 bp, Artificial Sequence
  Description of Artificial Sequence Synthetic oligonucleotide
gtttaagagctaagctggaaacagcatagcaagtttaaataaggctaggccgttatcaacttgaaaaagtggcaccgagtcggtgct

SEQ ID NO: 994 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcaggctgtggctgct

SEQ ID NO: 995 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcactgagtcagtgct

SEQ ID NO: 996 - DNA, 87 bp, Artificial Sequence
  Description of Artificial Sequence Synthetic oligonucleotide
gtttaagagctaagctggaaacagcatagcaagttttaataaggctagtccgttatcaacttgaaaaagtggcaccgagtcggtgct

SEQ ID NO: 997 - DNA, 87 bp, Artificial Sequence
  Description of Artificial Sequence Synthetic oligonucleotide
ggtttagagctaagctggaaacagcatagcaagtttaaataaggctagtccgttatcaacttgaaaaagtggcaccgagtcggtgct

SEQ ID NO: 998 - DNA, 87 bp, Artificial Sequence
  Description of Artificial Sequence Synthetic oligonucleotide
gtttaagagctaagctggaaacagcatggcaattttaaatacggctagtccgttatcaacttgaaaaagtggcaccgagtcggtgct

SEQ ID NO: 999 - DNA, 87 bp, Artificial Sequence
  Description of Artificial Sequence Synthetic oligonucleotide
gtttaagagctaagctggaaacagcatagcaaggttaaataaggctagtccgttatcaacctgaaaaggtggcaccgagtcggtgct

SEQ ID NO: 1000 - DNA, 87 bp, Artificial Sequence
  Description of Artificial Sequence Synthetic oligonucleotide
gtttaagagctaagctggaaacagcatagcaagtttaaatgaggctagtccgttatcaacttgacaaagtggcaccgagtcggtgct

SEQ ID NO: 1001 - DNA, 87 bp, Artificial Sequence
  Description of Artificial Sequence Synthetic oligonucleotide
gtttaagagctaagctggaaacagcatagcaagtttaaataacgctagtcggttatcaactggaaacagtggcgccgagtcggcgct

SEQ ID NO: 1002 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcaacgagtcgttgct

SEQ ID NO: 1003 - DNA, 87 bp, Artificial Sequence
  Description of Artificial Sequence Synthetic oligonucleotide
tttttagagctaagctggaaacagcatagcaagtttaaataaggctagtccgttatcaacttgaaaaagtggcaccgagtcggtgct

SEQ ID NO: 1004 - DNA, 87 bp, Artificial Sequence
  Description of Artificial Sequence Synthetic oligonucleotide
gtttaagagctaagctggaaacagcatagctaggttaaataaggctagtccgtgatcaacttgaaaaagtggcaccgagtcggtgct

SEQ ID NO: 1005 - DNA, 87 bp, Artificial Sequence
  Description of Artificial Sequence Synthetic oligonucleotide
gtttaagagctaagctggaaacagcatagcaagtttaaatgaggctagtccgttatcaacttgaaaaagtggcaccgaatcggtgct

SEQ ID NO: 1006 - DNA, 87 bp, Artificial Sequence
  Description of Artificial Sequence Synthetic oligonucleotide
gtttaagagctaagctggaaacagcatagcaagattaaataaggctagtccgtcatcgacttgaaaaagtggcaccgagtcggtgct

SEQ ID NO: 1007 - DNA, 87 bp, Artificial Sequence
  Description of Artificial Sequence Synthetic oligonucleotide
gtttaagagctaagctggaaacagcatagcaagtttaaattaggctagtccgttatcaacttgaaaaagtggcaccgcgtcggtgct

SEQ ID NO: 1008 - DNA, 87 bp, Artificial Sequence
  Description of Artificial Sequence Synthetic oligonucleotide
gctttagagctaagctggaaacagcatagcaagtttaaataaggctagtccgttatcaacttgaaaaagtggcaccgagtcggtgct

SEQ ID NO: 1009 - DNA, 87 bp, Artificial Sequence
  Description of Artificial Sequence Synthetic oligonucleotide
gtttaagagctaagctggaaacagcatagcaagtttaaataacgctagtcggttatcaacatgaaaatgtggctccgagtcggagct

SEQ ID NO: 1010 - DNA, 87 bp, Artificial Sequence
  Description of Artificial Sequence Synthetic oligonucleotide
gtttaagagctaagctggaaacagcataacaagtttaaataaggctagtcctttatcaacttgaaaaagtggcaccgagacggtgct

SEQ ID NO: 1011 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggccccgagtcggggct

SEQ ID NO: 1012 - DNA, 87 bp, Artificial Sequence
  Description of Artificial Sequence Synthetic oligonucleotide
gtttaagagctaagctggaaacagcatagcaaggttaaataaggctagtccgttatcaacatgaaaatgtggcaccgagtcggtgct

SEQ ID NO: 1013 - DNA, 87 bp, Artificial Sequence
  Description of Artificial Sequence Synthetic oligonucleotide
gtttaagagctaagctggaaacagcatagcaaggttaaataaggctagtccgttatcaagttgaaaaactggcaccgagtcggtgct

SEQ ID NO: 1014 - DNA, 87 bp, Artificial Sequence
  Description of Artificial Sequence Synthetic oligonucleotide
gtttaagagctaagctggaaacagcatagcaagtttaaataatgctagtcagttatcaacttgaaaaagtggcacagagtctgtgct

SEQ ID NO: 1015 - DNA, 87 bp, Artificial Sequence
  Description of Artificial Sequence Synthetic oligonucleotide
gtttaagagctaagctggaaacagcatagcaagtttaaataaaactagtttgttatcaacttgaaaaagtggcaccgagtcggtgct

SEQ ID NO: 1016 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcgccgagtcggcgct

SEQ ID NO: 1017 - DNA, 87 bp, Artificial Sequence
  Description of Artificial Sequence Synthetic oligonucleotide
gtttaagagctaagctggaaacagcatagcaaggttaaataaggctagtccgttatcaacttgagaaagtggcaccgagtcggtgct

SEQ ID NO: 1018 - DNA, 87 bp, Artificial Sequence
  Description of Artificial Sequence Synthetic oligonucleotide
gtttaagagctaagctggaaacagcatagcaagtttaaataaaggtactctgttatcaacttgaaaaagtggtaccgagtcggtact

SEQ ID NO: 1019 - DNA, 87 bp, Artificial Sequence
  Description of Artificial Sequence Synthetic oligonucleotide
gtttaagagctaagctggaaacagcatagcaagtttaaataaggctaggccgttatcaacttgataaagtggcaccgagtcggtgct

SEQ ID NO: 1020 - DNA, 87 bp, Artificial Sequence
  Description of Artificial Sequence Synthetic oligonucleotide
gtttaagagctaagctggaaacagcatagcaaggttaaataaggctagtccgttatcaaattgaaaaattggcaccgagtcggtgct

SEQ ID NO: 1021 - DNA, 87 bp, Artificial Sequence
  Description of Artificial Sequence Synthetic oligonucleotide
gtttaagagctaagctggaaacagcatagcaactttaaataagcctagtgcgttatcaacttgaaaaagtggcaccgagtcggtgct

SEQ ID NO: 1022 - DNA, 87 bp, Artificial Sequence
  Description of Artificial Sequence Synthetic oligonucleotide
gtttaagagctaagctggaaacagcatagcaaggttaaataacgctagtcggttatcaacttgaaaaagtggcaccgagtcggtgct

SEQ ID NO: 1023 - DNA, 87 bp, Artificial Sequence
  Description of Artificial Sequence Synthetic oligonucleotide
gtttaagagctaagctggaaacagcatagcaagtttaaataacgctagtcggttatcaacctgaaaaggtggcaccgagtcggtgct

SEQ ID NO: 1024 - DNA, 87 bp, Artificial Sequence
  Description of Artificial Sequence Synthetic oligonucleotide
gtttaagagctaagctggaaacagcatagcaaggttaaataaggctagtccgttatcaactggaaacagtggcaccgagtcggtgct

SEQ ID NO: 1025 - DNA, 87 bp, Artificial Sequence
  Description of Artificial Sequence Synthetic oligonucleotide
gtttaagagctaagctggaaacagcataacaagtttaaattaggctagtccgttatcaacttgaaaaagtggcaccgagtcggtgct

SEQ ID NO: 1026 - DNA, 87 bp, Artificial Sequence
  Description of Artificial Sequence Synthetic oligonucleotide
gtaaaagagctaagctggaaacagcatagcaagtttaaataaggctagtccgttatcaacttgaaaaagtggcaccgagtcggtgct

SEQ ID NO: 1027 - DNA, 87 bp, Artificial Sequence
  Description of Artificial Sequence Synthetic oligonucleotide
gtggaagagctaagctggaaacagcatagcaagtttaaataaggctagtccgttatcaacttgaaaaagtggcaccgagtcggtgct

SEQ ID NO: 1028 - DNA, 87 bp, Artificial Sequence
  Description of Artificial Sequence Synthetic oligonucleotide
gtttaagagctaagctggaaacagcatagcaaggttaaataaggctagtccgttatcaccttgaaaaaggggcaccgagtcggtgct

SEQ ID NO: 1029 - DNA, 87 bp, Artificial Sequence
  Description of Artificial Sequence Synthetic oligonucleotide
gtttaagagctaagctggaaacagcatagcaagtttaaataagggtactccgttatcaaattgaaaaattggcacccagtgggtgct

SEQ ID NO: 1030 - DNA, 87 bp, Artificial Sequence
  Description of Artificial Sequence Synthetic oligonucleotide
gtttaagagctaagctggaaacagcatagcaagtttaaataacgctagtcggttatcaacttgaaaaagtggcgctgagtcagcgct

SEQ ID NO: 1031 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggtaccgagtcggtact

SEQ ID NO: 1032 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtgacaccgagtcggtgtt

SEQ ID NO: 1033 - DNA, 87 bp, Artificial Sequence
  Description of Artificial Sequence Synthetic oligonucleotide
gtttaagagctaagctggaaacagcatagcaaaattaaataaggctagtccgttatcaacttgaaaaagtggcaccgagtcggtgct

SEQ ID NO: 1034 - DNA, 87 bp, Artificial Sequence
  Description of Artificial Sequence Synthetic oligonucleotide
gtttaagagctaagctggaaacagcatagcaaggttaaataaggctagtccggtatcaacttgaaaaagtggcaccgagtcggtgct

SEQ ID NO: 1035 - DNA, 87 bp, Artificial Sequence
  Description of Artificial Sequence Synthetic oligonucleotide
gtttaagagctaagctggaaacagcatagcaagtttaaataaatctagtatgttatcaacttgaaaaagtggcaccgagtcggtgct

SEQ ID NO: 1036 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcaacgagtcgttgct

SEQ ID NO: 1037 - DNA, 87 bp, Artificial Sequence
  Description of Artificial Sequence Synthetic oligonucleotide
gtttaagagctaagctggaaacagcatagcaagtttaaataatgctagtcagttatcaacttgaaaaagtggcaccaagttggtgct

SEQ ID NO: 1038 - DNA, 87 bp, Artificial Sequence
  Description of Artificial Sequence Synthetic oligonucleotide
gcttcagagctaagctggaaacagcatagcaagtttaaataaggctagtccgttatcaacttgaaaaagtggcaccgagtcggtgct

SEQ ID NO: 1039 - DNA, 87 bp, Artificial Sequence
  Description of Artificial Sequence Synthetic oligonucleotide
gtttaagagctaagctggaaacagcatagcgactttaaataaggctagtccgttatcaacttgagaaagtggcaccgagtcggtgct

SEQ ID NO: 1040 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcatcgagtcgatgct

SEQ ID NO: 1041 - DNA, 87 bp, Artificial Sequence
  Description of Artificial Sequence Synthetic oligonucleotide
gtccaagagctaagctggaaacagcatagcaagtttaaataaggctagtccgttatcaacttgaaaaagtggcaccgagtcggtgct

SEQ ID NO: 1042 - DNA, 87 bp, Artificial Sequence
  Description of Artificial Sequence Synthetic oligonucleotide
gtttaagagctaagctggaaacagcatagcaagtttaaataacgctagtcggttatcaacttgaaaaagtggcaccgagtcggtgct

SEQ ID NO: 1043 - DNA, 87 bp, Artificial Sequence
  Description of Artificial Sequence Synthetic oligonucleotide
gtttaagagctaagctggaaacagcatagcaagtttaaattaggctagtccgttatcaacttgaataagtggcaccgagtcggtgct

SEQ ID NO: 1044 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcagcgagtcgctgct

SEQ ID NO: 1045 - DNA, 87 bp, Artificial Sequence
  Description of Artificial Sequence Synthetic oligonucleotide
gtttaagagctaagctggaaacagcatagcaaggttaaataaggcttgtccgttatcaacttggaaaagtggcaccgagtcggtgct

SEQ ID NO: 1046 - DNA, 87 bp, Artificial Sequence
  Description of Artificial Sequence Synthetic oligonucleotide
gtttaagagctaagctggaaacagcatagcaagtttaaataacgctagtcggttatcaacttgaaaaagtggcgtcgagtcgacgct

SEQ ID NO: 1047 - DNA, 87 bp, Artificial Sequence
  Description of Artificial Sequence Synthetic oligonucleotide
gtttaagagctaagctggaaacagcatagcaagtttaaataacgctagtcggttatcaacttgagaaagtggcaccgagtcggtgct

SEQ ID NO: 1048 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcacagagtctgtgct

SEQ ID NO: 1049 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtgccaccgagtcggtggt

SEQ ID NO: 1050 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtgtcaccgagtcggtgat

SEQ ID NO: 1051 - DNA, 87 bp, Artificial Sequence
  Description of Artificial Sequence Synthetic oligonucleotide
gtttaagagctaagctggaaacagcatagcatgtttaaataaggctagtcctttatcaacttgaaaaagtggcaccgaggcggtgct

SEQ ID NO: 1052 - DNA, 87 bp, Artificial Sequence
  Description of Artificial Sequence Synthetic oligonucleotide
gtttaagagctaagctggaaacagcatagccgctttaaataaggctagtccgttatcaacttgaaaaagtggcaccgagtcggtgct

SEQ ID NO: 1053 - DNA, 87 bp, Artificial Sequence
  Description of Artificial Sequence Synthetic oligonucleotide
gtttaagagctaagctggaaacagcatagcaagcttaaataagtctagtacgttatcaacttgaaaaagtggcaccgagtcggtgct

SEQ ID NO: 1054 - DNA, 87 bp, Artificial Sequence
  Description of Artificial Sequence Synthetic oligonucleotide
ggtcaagagctaagctggaaacagcatagcaagtttaaataaggctagtccgttatcaacttgaaaaagtggcaccgagtcggtgct

SEQ ID NO: 1055 - DNA, 87 bp, Artificial Sequence
  Description of Artificial Sequence Synthetic oligonucleotide
ttgtaagagctaagctggaaacagcatagcaagtttaaataaggctagtccgttatcaacttgaaaaagtggcaccgagtcggtgct

SEQ ID NO: 1056 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcacagagtctgtgct

SEQ ID NO: 1057 - DNA, 87 bp, Artificial Sequence
  Description of Artificial Sequence Synthetic oligonucleotide
gtttaagagctaagctggaaacagcatagcaatattaaataaggctagtccgttatcaacttgaaaaagtggcaccgagtcggtgct

SEQ ID NO: 1058 - DNA, 87 bp, Artificial Sequence
  Description of Artificial Sequence Synthetic oligonucleotide
gtttaagagctaagctggaaacagcatagcaactttaaataaggttaatccgttatcaacttgaaaaagtggcaccgagtcggtgct

SEQ ID NO: 1059 - DNA, 87 bp, Artificial Sequence
  Description of Artificial Sequence Synthetic oligonucleotide
gtttaagagctaagctggaaacagcatagcaagtttaaattaggctagtccgttatcaacttgataaagtggcaccgagtcggtgct

SEQ ID NO: 1060 - DNA, 87 bp, Artificial Sequence
  Description of Artificial Sequence Synthetic oligonucleotide
gtttaagagctaagctggaaacagcatagcaagtttaaataacggtactcggttatcaacttgaaaaagtggcaccgagtcggtgct

SEQ ID NO: 1061 - DNA, 87 bp, Artificial Sequence
  Description of Artificial Sequence Synthetic oligonucleotide
gtttaagagctaagctggaaacagcatagcaaggttaaataaggctagtccgttatcaatttgaaaaaatggcaccgagtcggtgct

SEQ ID NO: 1062 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcacctagtaggtgct

SEQ ID NO: 1063 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggcatccagttgctgct

SEQ ID NO: 1064 - DNA, 87 bp, Artificial Sequence
  Description of Artificial Sequence Synthetic oligonucleotide
gtttaagagctaagctggaaacagcatagcaagtttaaataacgctagtcggttatcaacttgaaaaagtggcaacgagtcgttgct

SEQ ID NO: 1065 - DNA, 87 bp, Artificial Sequence
  Description of Artificial Sequence Synthetic oligonucleotide
gtgtaagtgctaagctggaaacagcatagcaagtttaaataaggctagtccgttatcaacttgaaaaagtggcaccgagtcggtgct

SEQ ID NO: 1066 - DNA, 87 bp, Artificial Sequence
  Description of Artificial Sequence Synthetic oligonucleotide
gtttaagagctaagctggaaacagcatagcaagcttaaataaagctagtctgttatcaacttgaaaaagtggcaccgagtcggtgct

SEQ ID NO: 1067 - DNA, 87 bp, Artificial Sequence
  Description of Artificial Sequence Synthetic oligonucleotide
gtttaagagctaagctggaaacagcatagcaaggttaaataaggctagtccgttatcaacttgataaagtggcaccgagtcggtgct

SEQ ID NO: 1068 - DNA, 87 bp, Artificial Sequence
  Description of Artificial Sequence Synthetic oligonucleotide
gtttaagagctaagctggaaacagcatagcaagtttaaataaggctagaccgttatcaacttgaaaaagtcgcaccgaggcggtgct

SEQ ID NO: 1069 - DNA, 87 bp, Artificial Sequence
  Description of Artificial Sequence Synthetic oligonucleotide
cttgaagagctaagctggaaacagcatagcaagtttaaataaggctagtccgttatcaacttgaaaaagtggcaccgagtcggtgct

SEQ ID NO: 1070 - DNA, 87 bp, Artificial Sequence
  Description of Artificial Sequence Synthetic oligonucleotide
gtttaagagctaagctggaaacagcatagcaagtttaaataatgctagtcagttatcaacttgaaaaagtgtctccgagtcggagat

SEQ ID NO: 1071 - DNA, 87 bp, Artificial Sequence
  Description of Artificial Sequence Synthetic oligonucleotide
gtttaagagctaagctggaaacagcatagcaagtttaaataagcctagtccgttatcaacttgaaaaagtggcaccgagtcggtgct

SEQ ID NO: 1072 - DNA, 87 bp, Artificial Sequence
  Description of Artificial Sequence Synthetic oligonucleotide
gtttaagagctaagctggaaacagcatagcaactttaaataaggctagtccgttatcatcttgaaaaagaggcaccgagtcggtgct

SEQ ID NO: 1073 - DNA, 87 bp, Artificial Sequence
  Description of Artificial Sequence Synthetic oligonucleotide
gtttaagagctaagctggaaacagcatagcatttttaaataaggctagtccgttatcaacttgaaaaagtggcaccgagtcggtgct

SEQ ID NO: 1074 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggccccgagtcggggct

SEQ ID NO: 1075 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcaccaagttggtgct

SEQ ID NO: 1076 - DNA, 87 bp, Artificial Sequence
  Description of Artificial Sequence Synthetic oligonucleotide
atgtaagagctaagctggaaacagcatagcaagtttaaataaggctagtccgttatcaacttgaaaaagtggcaccgagtcggtgct

SEQ ID NO: 1077 - DNA, 87 bp, Artificial Sequence
  Description of Artificial Sequence Synthetic oligonucleotide
attaaagagctaagctggaaacagcatagcaagtttaaataaggctagtccgttatcaacttgaaaaagtggcaccgagtcggtgct

SEQ ID NO: 1078 - DNA, 87 bp, Artificial Sequence
  Description of Artificial Sequence Synthetic oligonucleotide
gtttaagagctaagctggaaacagcatagcaagtttaaataatgttaatcagttatcaacttgaaaaagtggccccgagtcggggct

SEQ ID NO: 1079 - DNA, 87 bp, Artificial Sequence
  Description of Artificial Sequence Synthetic oligonucleotide
gtttaagagctaagctggaaacagcatagcaagtttaaatagggctagtcctttatcaacttgcaaaagtggcaccgagtcggtgct

SEQ ID NO: 1080 - DNA, 87 bp, Artificial Sequence
  Description of Artificial Sequence Synthetic oligonucleotide
gtttaagagctaagctggaaacagcatagcaactttaaataagtctagtacgttatcaacttgaaaaagtggcaccgagtcggtgct

SEQ ID NO: 1081 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtgggaccgagtcggtcct

SEQ ID NO: 1082 - DNA, 87 bp, Artificial Sequence
  Description of Artificial Sequence Synthetic oligonucleotide
gtttaagagctaagctggaaacagcatagcaaggttaaataaggctagtccgttatcatcttgaaaaagaggcaccgagtcggtgct

SEQ ID NO: 1083 - DNA, 87 bp, Artificial Sequence
  Description of Artificial Sequence Synthetic oligonucleotide
gtttaagagctaagctggaaacagcatatcacctttaaataaggctagtccgttatcaacttgaaaaagtggcaccgagtcggtgct

SEQ ID NO: 1084 - DNA, 87 bp, Artificial Sequence
  Description of Artificial Sequence Synthetic oligonucleotide
gtttaagagctaagctggaaacagcatagcaatcttaaataaggctagtccgttatcaacttgaaaaagtggcaccgagtcggtgct

SEQ ID NO: 1085 - DNA, 87 bp, Artificial Sequence
  Description of Artificial Sequence Synthetic oligonucleotide
gtttaagagctaagctggaaacagcatagcaagtttaaattaggctagtccgttatcaacttgaaaaagtggcaccgtgtcggtgct

SEQ ID NO: 1086 - DNA, 87 bp, Artificial Sequence
  Description of Artificial Sequence Synthetic oligonucleotide
gtttaagagctaagctggaaacagcatagcaaccttaaataaggctagtccgttatcaacttgaaaaagtggcaccgagtcggtgct

SEQ ID NO: 1087 - DNA, 87 bp, Artificial Sequence
  Description of Artificial Sequence Synthetic oligonucleotide
gtttaagagctaagctggaaacagcatagcaaggttaaataagcctagtgcgttatcaacttgaaaaagtggcaccgagtcggtgct

SEQ ID NO: 1088 - DNA, 87 bp, Artificial Sequence
  Description of Artificial Sequence Synthetic oligonucleotide
gtttaagagctaagctggaaacagcatagcaagtttaaataaggctagtcccttatcaacttgaaatagtggcaccgagtcggtgct

SEQ ID NO: 1089 - DNA, 87 bp, Artificial Sequence
  Description of Artificial Sequence Synthetic oligonucleotide
gtttaagagctaagctggaaacagcatagcaactttaaataaggctagtccgatatcaacttgaaaaagtggcaccgaggcggtgct

SEQ ID NO: 1090 - DNA, 87 bp, Artificial Sequence
  Description of Artificial Sequence Synthetic oligonucleotide
gtttaagagctaagctggaaacagcatagcaagtttaaataaggctagtcggttatcaacttgaaaaagtggcaccgagtcggtgct

SEQ ID NO: 1091 - DNA, 87 bp, Artificial Sequence
  Description of Artificial Sequence Synthetic oligonucleotide
gtttaagagctaagctggaaacagcatagcaagtttaaataaccctagtgggttatcaacttgaaaaagtggcaccgagtcggtgct

SEQ ID NO: 1092 - DNA, 87 bp, Artificial Sequence
  Description of Artificial Sequence Synthetic oligonucleotide
gtttaagagctaagctggaaacagcatagcaattttaaataaggctagtccgttatcaacttgaaaaagtagcaccgagtcggtgct

SEQ ID NO: 1093 - DNA, 87 bp, Artificial Sequence
  Description of Artificial Sequence Synthetic oligonucleotide
gtttaagagctaagctggaaacagcatagcaagtttaaatagggctagtcggttatcaacttgaaaaagtggcaccgagtcggtgct

SEQ ID NO: 1094 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcacggagtccgtgct

SEQ ID NO: 1095 - DNA, 87 bp, Artificial Sequence
  Description of Artificial Sequence Synthetic oligonucleotide
gtttaagagctaagctggaaacagcatagcaattttaaatgaggctagtccgttatcaacttgaaaaagtggcaccgagtcggtgct

SEQ ID NO: 1096 - DNA, 87 bp, Artificial Sequence
  Description of Artificial Sequence Synthetic oligonucleotide
gtgtaggagctaagctggaaacagcatagcaagtttaaataaggctagtccgttatcaacttgaaaaagtggcaccgagtcggtgct

SEQ ID NO: 1097 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtggctggcagtccgagct

SEQ ID NO: 1098 - DNA, 87 bp, Artificial Sequence
  Description of Artificial Sequence Synthetic oligonucleotide
gtttaagagctaagctggaaacagcatagcaagtttaaataacgctagtcggttatcaccttgaaaaaggggcacctagtaggtgct

SEQ ID NO: 1099 - DNA, 87 bp, Artificial Sequence
  Description of Artificial Sequence Synthetic oligonucleotide
gtttaagagctaagctggaaacagcataccaagtttaaataaggctaggccgttatcaacttgaaaaagtggcaccgattcggtgct

SEQ ID NO: 1100 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcacggagtccgtgct

SEQ ID NO: 1101 - DNA, 87 bp, Artificial Sequence
  Description of Artificial Sequence Synthetic oligonucleotide
gtttaagagctaagctggaaacagcatagcaagtttaaataacgctagtcggttatcaacttgaaaaagtggaaccaagttggttct

SEQ ID NO: 1102 - DNA, 87 bp, Artificial Sequence
  Description of Artificial Sequence Synthetic oligonucleotide
gtttaagagctaagctggaaacagcatagccactttaaataaggctagtccgttatcaacttgaaaaagtggcaccgagtcggtgct

SEQ ID NO: 1103 - DNA, 87 bp, Artificial Sequence
  Description of Artificial Sequence Synthetic oligonucleotide
gtttaagagctaagctggaaacagcatagcaagattaaatcaggctagtccgttatcaacttgtaaaagtggcaccgagtcggtgct

SEQ ID NO: 1104 - DNA, 87 bp, Artificial Sequence
  Description of Artificial Sequence Synthetic oligonucleotide
gtttaagagctaagctggaaacagcatagcaagtttaaataaggctagtccgttatcaacttgaaaaagtgcgtgcgagtcggaggt

SEQ ID NO: 1105 - DNA, 87 bp, Artificial Sequence
  Description of Artificial Sequence Synthetic oligonucleotide
gtttaagagctaagctggaaacagcatagcaagtttaaatgaggctagcccgttatcaacttgaaaaagtggcaccgagtcggtgct

SEQ ID NO: 1106 - DNA, 87 bp, Artificial Sequence
  Description of Artificial Sequence Synthetic oligonucleotide
gtttaagagctaagctggaaacagcatagcaagtttaaatatggctagtcctttatcaacttgaaaaagtggcaccgagtcggtgct

SEQ ID NO: 1107 - DNA, 87 bp, Artificial Sequence
  Description of Artificial Sequence Synthetic oligonucleotide
gctcaagagctaagctggaaacagcatagcaagtttaaataaggctagtccgttatcaacttgaaaaagtggcaccgagtcggtgct

SEQ ID NO: 1108 - DNA, 87 bp, Artificial Sequence
  Description of Artificial Sequence Synthetic oligonucleotide
gtttaagagctaagctggaaacagcatggcaaggttaaataaggcgagtccgttatcaacttgaaaaagtggcaccgagtcggtgct

SEQ ID NO: 1109 - DNA, 87 bp, Artificial Sequence
  Description of Artificial Sequence Synthetic oligonucleotide
gtttaagagctaagctggaaacagcatagcaagcttaaattaggctagtccgttatcaacttgaaaaagtggcaccgagtcggtgct

SEQ ID NO: 1110 - DNA, 87 bp, Artificial Sequence
  Description of Artificial Sequence Synthetic oligonucleotide
gtgcaagagctaagctggaaacagcatagcaagtttaaataaggctagtccgttatcaacttgaaaaagtggcaccgagtcggtgct

SEQ ID NO: 1111 - DNA, 87 bp, Artificial Sequence
  Description of Artificial Sequence Synthetic oligonucleotide
gtttaagagctaagctggaaacagcatagcaaggttaaataaggctaggccgttatcaacttgaaaaagtggcaccgagtcggtgct

SEQ ID NO: 1112 - DNA, 87 bp, Artificial Sequence
  Description of Artificial Sequence Synthetic oligonucleotide
gtttaagagctaagctggaaacagcatagcaaggttaaataaggttaatccgttatcaacttgaaaaagtggcaccgagtcggtgct

SEQ ID NO: 1113 - DNA, 87 bp, Artificial Sequence
  Description of Artificial Sequence Synthetic oligonucleotide
gtttaagagctaagctggaaacagcatagcaagtttaaataaggctaggccgttatcaacttgaaaaagtcgcaccgagtcggtgct

SEQ ID NO: 1114 - DNA, 87 bp, Artificial Sequence
  Description of Artificial Sequence Synthetic oligonucleotide
gtttaagagctaagctggaaacagcatagcaacgttaaataaggctagtccgttatcaacttgaaaaagtggcaccgagtcggtgct

SEQ ID NO: 1115 - DNA, 87 bp, Artificial Sequence
  Description of Artificial Sequence Synthetic oligonucleotide
gtttaagagctaagctggaaacagcatagcaaggttaaataaagctagtctgttatcaacttgaaaaagtggcaccgagtcggtgct

SEQ ID NO: 1116 - DNA, 87 bp, Artificial Sequence
  Description of Artificial Sequence Synthetic oligonucleotide
gtttaagagctaagctggaaacagcatagcaaggttaaataaggatattccgttatcaacttgaaaaagtggcaccgagtcggtgct

SEQ ID NO: 1117 - DNA, 87 bp, Artificial Sequence
  Description of Artificial Sequence Synthetic oligonucleotide
gatttagagctaagctggaaacagcatagcaagtttaaataaggctagtccgttatcaacttgaaaaagtggcaccgagtcggtgct

SEQ ID NO: 1118 - DNA, 87 bp, Artificial Sequence
  Description of Artificial Sequence Synthetic oligonucleotide
gtttaagagctaagctggaaacagcatagcaactttaaataaggctagtcctttatcaacttgaaaaagtggcaccgagtcggtgct

SEQ ID NO: 1119 - DNA, 87 bp, Artificial Sequence
  Description of Artificial Sequence Synthetic oligonucleotide
gtttaagagctaagctggaaacagcatagcaagtttaaataacgctagtcggttatcatcttgaaaaagaggaaccgagtcggttct

SEQ ID NO: 1120 - DNA, 87 bp, Artificial Sequence
  Description of Artificial Sequence Synthetic oligonucleotide
gtttaagagctaagctggaaacagcatagcaagtttaaattaggctagtccgttatcaacttgaaaaagtcgcaccgagccggtgct

SEQ ID NO: 1121 - DNA, 87 bp, Artificial Sequence
  Description of Artificial Sequence Synthetic oligonucleotide
gtttaagagctaagctggaaacagcatcgcaactttaaataaggctagtccgttatcaacttgaaaaagtcgcaccgagtcggtgct

SEQ ID NO: 1122 - DNA, 87 bp, Artificial Sequence
  Description of Artificial Sequence Synthetic oligonucleotide
gtttaagagctaagctggaaacagcatagcaagtttaaataatgctagtccgttatcaacttgaaaaagtggcaccgagtcggtgct

SEQ ID NO: 1123 - DNA, 87 bp, Artificial Sequence
  Description of Artificial Sequence Synthetic oligonucleotide
gtttaagagctaagctggaaacagcatagcaactttaaataaggatattccgttatcaacttgaaaaagtggcaccgagtcggtgct

SEQ ID NO: 1124 - DNA, 87 bp, Artificial Sequence
  Description of Artificial Sequence Synthetic oligonucleotide
gtttaagagctaagctggaaacagcatagcaaggttaaataagactagttcgttatcaacttgaaaaagtggcaccgagtcggtgct

SEQ ID NO: 1125 - DNA, 87 bp, Artificial Sequence
  Description of Artificial Sequence Synthetic oligonucleotide
gtttaagagctaagctggaaacagcatagcaactttaaataaggcttgtccgttatcaacttgaaaaagttgcaccgagtcggtgct

SEQ ID NO: 1126 - DNA, 87 bp, Artificial Sequence
  Description of Artificial Sequence Synthetic oligonucleotide
gtttaagagctaagctggaaacagcatagcaagtttaaataacgctagtcggttatcaactggaaacagtggcaccgagtcggtgct

SEQ ID NO: 1127 - DNA, 87 bp, Artificial Sequence
  Description of Artificial Sequence Synthetic oligonucleotide
gtttaagagctaagctggaaacagcatagcaagtttaaataaggctagtcccttatcaacttgaaaaagtggcaccgagtcggtgct

SEQ ID NO: 1128 - DNA, 87 bp, Artificial Sequence
  Description of Artificial Sequence Synthetic oligonucleotide
gtgttagagctaagctggaaacagcatagcaagtttaaataaggctagtccgttatcaacttgaaaaagtggcaccgagtcggtgct

SEQ ID NO: 1129 - DNA, 87 bp, Artificial Sequence
  Description of Artificial Sequence Synthetic oligonucleotide
gtttaagagctaagctggaaacagcatagcaagtttaaattaggctcgtccgttatcaacttgacaaagtggcaccgagtcggtgct

SEQ ID NO: 1130 - DNA, 87 bp, Artificial Sequence
  Description of Artificial Sequence Synthetic oligonucleotide
gtttaagagctaagctggaaacagcatagcaagtttaaataaggcttgtcccttatcaacttgaaaaagtggcaccgagtcggtgct

SEQ ID NO: 1131 - DNA, 87 bp, Artificial Sequence
  Description of Artificial Sequence Synthetic oligonucleotide
gttagagagctaagctggaaacagcatagcaagtttaaataaggctagtccgttatcaacttgaaaaagtggcaccgagtcggtgct

SEQ ID NO: 1132 - DNA, 87 bp, Artificial Sequence
  Description of Artificial Sequence Synthetic oligonucleotide
gtttaagagctaagctggaaacagcatagcaagtttaaataacgctagtcggttatcatcttgaaaaagaggcaccgagtcggtgct

SEQ ID NO: 1133 - DNA, 87 bp, Artificial Sequence
  Description of Artificial Sequence Synthetic oligonucleotide
gtttaagagctaagctggaaacagcatagcaagtttaaataaccgaactcggttatcaacttgaaaaagtggcaccgagtcggtgct

SEQ ID NO: 1134 - DNA, 87 bp, Artificial Sequence
  Description of Artificial Sequence Synthetic oligonucleotide
gtttaagagctaagctggaaacagcatagcaagtttaaataatgctattccgttatcaacttgaaaaagtggcaccgagtcggtgct

SEQ ID NO: 1135 - DNA, 87 bp, Artificial Sequence
  Description of Artificial Sequence Synthetic oligonucleotide
gtttaagagctaagctggaaacagcatagcaagtttaaataaggctagtgcgttatcaacttgaaaaagtggcaccgagtcggtgct

SEQ ID NO: 1136 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtcgcaccgagtcggtgct

SEQ ID NO: 1137 - DNA, 87 bp, Artificial Sequence
  Description of Artificial Sequence Synthetic oligonucleotide
gtttaagagctaagctggaaacagcatagcaactttaaataagggtactccgttatcaacttgaaaaagtggcaccgagtcggtgct

SEQ ID NO: 1138 - DNA, 87 bp, Artificial Sequence
  Description of Artificial Sequence Synthetic oligonucleotide
gtttaagagctaagctggaaacagcatagcaagtttaaataaggctagtcccttatcaacttgaaaaagtggcaccgaatcggtgct

SEQ ID NO: 1139 - DNA, 87 bp, Artificial Sequence
  Description of Artificial Sequence Synthetic oligonucleotide
gtttaagagctaagctggaaacagcatagcaactttaaatatggctagtccgttatcaacttgaaaaagtggcaccgagtcggtgct

SEQ ID NO: 1140 - DNA, 87 bp, Artificial Sequence
  Description of Artificial Sequence Synthetic oligonucleotide
gtttaagagctaagctggaaacagcatagcaaggttaaataatgctagtcagttatcaacttgaaaaagtggcaccgagtcggtgct

SEQ ID NO: 1141 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcacccagtgggtgct

SEQ ID NO: 1142 - DNA, 87 bp, Artificial Sequence
  Description of Artificial Sequence Synthetic oligonucleotide
gtttaagagctaagctggaaacagcatagcaaggttaaataaggctagtccgttatcaactagaaatagtggcaccgagtcggtgct

SEQ ID NO: 1143 - DNA, 87 bp, Artificial Sequence
  Description of Artificial Sequence Synthetic oligonucleotide
gtttaagagctaagctggaaacagcatagcaagtttaaattaggctagtccgttgtgaacttgaaaaagtggcaccgagtcggtgct

SEQ ID NO: 1144 - DNA, 87 bp, Artificial Sequence
  Description of Artificial Sequence Synthetic oligonucleotide
gtttaagagctaagctggaaacagcatagcatgtttaaataaggctaggccgttattaacttgaaaaagtggcaccgagtcggtgct

SEQ ID NO: 1145 - DNA, 87 bp, Artificial Sequence
  Description of Artificial Sequence Synthetic oligonucleotide
gtttaagagctaagctggaaacagcatagcaagtttaaataacgctagtcggttatcaacttgaaaaagtgtcaccgagtcggtgat

SEQ ID NO: 1146 - DNA, 87 bp, Artificial Sequence
  Description of Artificial Sequence Synthetic oligonucleotide
gtttaagagctaagctggaaacagcatagcaactttaaataagactagttcgttatcaacttgaaaaagtggcaccgagtcggtgct

SEQ ID NO: 1147 - DNA, 87 bp, Artificial Sequence
  Description of Artificial Sequence Synthetic oligonucleotide
gtttaagagctaagctggaaacagcatagcaaggttaaataagggtactccgttatcaacttgaaaaagtggcaccgagtcggtgct

SEQ ID NO: 1148 - DNA, 87 bp, Artificial Sequence
  Description of Artificial Sequence Synthetic oligonucleotide
gtttcggagctaagctggaaacagcatagcaagtttaaataaggctagtccgttatcaacttgaaaaagtggcaccgagtcggtgct

SEQ ID NO: 1149 - DNA, 87 bp, Artificial Sequence
  Description of Artificial Sequence Synthetic oligonucleotide
gtttaagagctaagctggaaacagcatagcaagcttaaataatgctagtcagttatcaacttgaaaaagtggcaccgagtcggtgct

SEQ ID NO: 1150 - DNA, 87 bp, Artificial Sequence
  Description of Artificial Sequence Synthetic oligonucleotide
gtgtgagagctaagctggaaacagcatagcaagtttaaataaggctagtccgttatcaacttgaaaaagtggcaccgagtcggtgct

SEQ ID NO: 1151 - DNA, 87 bp, Artificial Sequence
  Description of Artificial Sequence Synthetic oligonucleotide
gtttaagagctaagctggaaacagcatagcaaggttaaataagtctagtacgttatcaacttgaaaaagtggcaccgagtcggtgct

SEQ ID NO: 1152 - DNA, 87 bp, Artificial Sequence
  Description of Artificial Sequence Synthetic oligonucleotide
gtttaagagctaagctggaaacagcatagcaactttaaataacgctagtcggttatcaacttgaaaaagtggcaccgagtcggtgct

SEQ ID NO: 1153 - DNA, 87 bp, Artificial Sequence
  Description of Artificial Sequence Synthetic oligonucleotide
gttgatgagctaagctggaaacagcatagcaagtttaaataaggctagtccgttatcaacttgaaaaagtggcaccgagtcggtgct

SEQ ID NO: 1154 - DNA, 87 bp, Artificial Sequence
  Description of Artificial Sequence Synthetic oligonucleotide
gtttaagagctaagctggaaacagcatagcaattttaaataaggctaggccgttatcaacttgaaaaagtggcaccgagtcggtgct

SEQ ID NO: 1155 - DNA, 87 bp, Artificial Sequence
  Description of Artificial Sequence Synthetic oligonucleotide
gtttaagagctaagctggaaacagcatagcaactttaaataaggcaagtcccttatcaacttgaaaaagtggcaccgagtcggtgct

SEQ ID NO: 1156 - DNA, 87 bp, Artificial Sequence
  Description of Artificial Sequence Synthetic oligonucleotide
gtttaagagctaagctggaaacagcatagcaagcttaaatgaggctagtccggtatcaacttgaaaaagtggcaccgagtcggtgct

SEQ ID NO: 1157 - DNA, 87 bp, Artificial Sequence
  Description of Artificial Sequence Synthetic oligonucleotide
gtttaagagctaagctggaaacagcatagcaactttaaataaggctagtccgttatcaaattgaaaaattggcaccgagtcggtgct

SEQ ID NO: 1158 - DNA, 87 bp, Artificial Sequence
  Description of Artificial Sequence Synthetic oligonucleotide
gtttaagagctaagctggaaacagcatagcaaggttaaataaggctagtccgttatcaacttgagaaagtggcaccgcgtcggtgct

SEQ ID NO: 1159 - DNA, 87 bp, Artificial Sequence
  Description of Artificial Sequence Synthetic oligonucleotide
gtttaagagctaagctggaaacagcatagcaagtttaaataaggctagtcccatatcaacttgaaaaagtggcaccgagtcggtgct

SEQ ID NO: 1160 - DNA, 87 bp, Artificial Sequence
  Description of Artificial Sequence Synthetic oligonucleotide
gtttaagagctaagctggaaacagcatagcaagtttaaataaggctagtcagttatcaacttgaaaaagtggcaccgagtcggtgct

SEQ ID NO: 1161 - DNA, 87 bp, Artificial Sequence
  Description of Artificial Sequence Synthetic oligonucleotide
gtttaagagctaagctggaaacagcatcgcaactttaaataaggctagtcctttatcaacttgaaaaagtggcaccgagtcggtgct

SEQ ID NO: 1162 - DNA, 87 bp, Artificial Sequence
  Description of Artificial Sequence Synthetic oligonucleotide
gattatgagctaagctggaaacagcatagcaagtttaaataaggctagtccgttatcaacttgaaaaagtggcaccgagtcggtgct

SEQ ID NO: 1163 - DNA, 87 bp, Artificial Sequence
  Description of Artificial Sequence Synthetic oligonucleotide
gtttaagagctaagctggaaacagcatagcaattttaaatgaggctagtccgttatcgacttgaaaaagtggcaccgagtcggtgct

SEQ ID NO: 1164 - DNA, 87 bp, Artificial Sequence
  Description of Artificial Sequence Synthetic oligonucleotide
gtttaagagctaagctggaaacagcatagcaagcttaaataacgctagtcggttatcaacttgaaaaagtggcaccgagtcggtgct

SEQ ID NO: 1165 - DNA, 87 bp, Artificial Sequence
  Description of Artificial Sequence Synthetic oligonucleotide
gtttaagagctaagctggaaacagcatatcaagattaaataaggctagtccgttatcaacttgagaaagtggcaccgagtcggtgct

SEQ ID NO: 1166 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcagcgagtcgctgct

SEQ ID NO: 1167 - DNA, 87 bp, Artificial Sequence
  Description of Artificial Sequence Synthetic oligonucleotide
gtttaagagctaagctggaaacagcatagcaaagttaaataaggctagtccgttatcaacttgaaaaagtggcaccgagtcggtgct

SEQ ID NO: 1168 - DNA, 87 bp, Artificial Sequence
  Description of Artificial Sequence Synthetic oligonucleotide
gtttaagagctaagctggaaacagcatagcaaggttaaatagggctagtccgttatcaacttgataaagtggcaccgagtcggtgct

SEQ ID NO: 1169 - DNA, 87 bp, Artificial Sequence
  Description of Artificial Sequence Synthetic oligonucleotide
gtttaagagctaagctggaaacagcatagcaagcttaaataaggctagtccgttatcaacttgaaaaagtggcacccagtgggtgct

SEQ ID NO: 1170 - DNA, 87 bp, Artificial Sequence
  Description of Artificial Sequence Synthetic oligonucleotide
gtttaagagctaagctggaaacagcatagcaagtttaaataacgctagtccgttatcaacttgaaaaagtggcaccgagtcggtgct

SEQ ID NO: 1171 - DNA, 87 bp, Artificial Sequence
  Description of Artificial Sequence Synthetic oligonucleotide
gtatacgagctaagctggaaacagcatagcaagtttaaataaggctagtccgttatcaacttgaaaaagtggcaccgagtcggtgct

SEQ ID NO: 1172 - DNA, 87 bp, Artificial Sequence
  Description of Artificial Sequence Synthetic oligonucleotide
gttgcagagctaagctggaaacagcatagcaagtttaaataaggctagtccgttatcaacttgaaaaagtggcaccgagtcggtgct

SEQ ID NO: 1173 - DNA, 87 bp, Artificial Sequence
  Description of Artificial Sequence Synthetic oligonucleotide
gtttaagagctaagctggaaacagcatagcaactttaaataaggctagtccgttatcaatttgaaaaaatggcaccgagtcggtgct

SEQ ID NO: 1174 - DNA, 87 bp, Artificial Sequence
  Description of Artificial Sequence Synthetic oligonucleotide
gtttaagagctaagctggaaacagcatagcaactttaaataaggctagtccgttattaacttgataaagtggcaccgagtcggtgct

SEQ ID NO: 1175 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcacccagtgggtgct

SEQ ID NO: 1176 - DNA, 87 bp, Artificial Sequence
  Description of Artificial Sequence Synthetic oligonucleotide
gattcagagctaagctggaaacagcatagcaagtttaaataaggctagtccgttatcaacttgaaaaagtggcaccgagtcggtgct

SEQ ID NO: 1177 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcaccgagacggtgct

SEQ ID NO: 1178 - DNA, 87 bp, Artificial Sequence
  Description of Artificial Sequence Synthetic oligonucleotide
gtttaagagctaagctggaaacagcatagcaaggttaaataaggctagtccgttatcaacttgaaaaagtggcactgagtcagtgct

SEQ ID NO: 1179 - DNA, 87 bp, Artificial Sequence
  Description of Artificial Sequence Synthetic oligonucleotide
gttaaaaagctaagctggaaacagcatagcaagtttaaataaggctagtccgttatcaacttgaaaaagtggcaccgagtcggtgct

SEQ ID NO: 1180 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcaccgtgtcggtgct

SEQ ID NO: 1181 - DNA, 87 bp, Artificial Sequence
  Description of Artificial Sequence Synthetic oligonucleotide
gtttaagagctaagctggaaacagcatagcaagtttaaataatgatattcagttatcaacttgaaaaagtggcaccgagtcggtgct

SEQ ID NO: 1182 - DNA, 87 bp, Artificial Sequence
  Description of Artificial Sequence Synthetic oligonucleotide
gtttaagagctaagctggaaacagcatagcaagtttaaataacgctagtcggttatcaagttgaaaaactggcagcgagtcgctgct

SEQ ID NO: 1183 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcacctagtaggtgct

SEQ ID NO: 1184 - DNA, 87 bp, Artificial Sequence
  Description of Artificial Sequence Synthetic oligonucleotide
gctaaagagctaagctggaaacagcatagcaagtttaaataaggctagtccgttatcaacttgaaaaagtggcaccgagtcggtgct

SEQ ID NO: 1185 - DNA, 87 bp, Artificial Sequence
  Description of Artificial Sequence Synthetic oligonucleotide
gcataagagctaagctggaaacagcatagcaagtttaaataaggctagtccgttatcaacttgaaaaagtggcaccgagtcggtgct

SEQ ID NO: 1186 - DNA, 87 bp, Artificial Sequence
  Description of Artificial Sequence Synthetic oligonucleotide
gtttaagagctaagctggaaacagcatagcaactttaaataaagctagtctgttatcaacttgaaaaagtggcaccgagtcggtgct

SEQ ID NO: 1187 - DNA, 87 bp, Artificial Sequence
  Description of Artificial Sequence Synthetic oligonucleotide
gtttaagagctaagctggaaacagcatagcaagtttaaataacgctagtcggttatcaaattgaaaaattggcgccgagtcggcgct

SEQ ID NO: 1188 - DNA, 87 bp, Artificial Sequence
  Description of Artificial Sequence Synthetic oligonucleotide
gtgtcagagctaagctggaaacagcatagcaagtttaaataaggctagtccgttatcaacttgaaaaagtggcaccgagtcggtgct

SEQ ID NO: 1189 - DNA, 87 bp, Artificial Sequence
  Description of Artificial Sequence Synthetic oligonucleotide
gtttaagagctaagctggaaacagcatagcaagtttaaatcaggctagtccattatctacttgaaaaagtggcaccgagtcggtgct

SEQ ID NO: 1190 - DNA, 87 bp, Artificial Sequence
  Description of Artificial Sequence Synthetic oligonucleotide
gtttaagagctaagctggaaacagcataacaagtttaaataaggctagtcccttatcaacttgaataagtggcaccgagtcggtgct

SEQ ID NO: 1191 - DNA, 87 bp, Artificial Sequence
  Description of Artificial Sequence Synthetic oligonucleotide
gtttaagagctaagctggaaacagcatagcaagtttaaataatcctagttcgttatcaacttgaaaaagtggcaccgagtcggtgct

SEQ ID NO: 1192 - DNA, 87 bp, Artificial Sequence
  Description of Artificial Sequence Synthetic oligonucleotide
gtttaagagctaagctggaaacagcatagcaactttaaataatgctagtcagttatcaacttgaaaaagtggcaccgagtcggtgct

SEQ ID NO: 1193 - DNA, 87 bp, Artificial Sequence
  Description of Artificial Sequence Synthetic oligonucleotide
gtttaagagctaagctggaaacagcatatcaagcttaaataaggctagtccgttatgaacttgaaaaagtggcaccgagtcggtgct

SEQ ID NO: 1194 - DNA, 87 bp, Artificial Sequence
  Description of Artificial Sequence Synthetic oligonucleotide
gtttaagagctaagctggaaacagcatagcaactttaaataaggctagtccgttatcaacttgaaaaagtggcaccaagttggtgct

SEQ ID NO: 1195 - DNA, 87 bp, Artificial Sequence
  Description of Artificial Sequence Synthetic oligonucleotide
gtttaagagctaagctggaaacagcttagcaagtttaaataaggctagtccgttatcaacttgaaaaagtggcaccgagtcggtgct

SEQ ID NO: 1196 - RNA, 20 nt, Artificial Sequence
  Description of Artificial Sequence Synthetic oligonucleotide
gaccaggaugggcaccaccc

SEQ ID NO: 1197 - RNA, 20 nt, Artificial Sequence
  Description of Artificial Sequence Synthetic oligonucleotide
gaccgggaugggcaccaccc

SEQ ID NO: 1198 - RNA, 20 nt, Artificial Sequence
  Description of Artificial Sequence Synthetic oligonucleotide
gaccagguugggcaccaccc

SEQ ID NO: 1199 - RNA, 20 nt, Artificial Sequence
  Description of Artificial Sequence Synthetic oligonucleotide
gaccaggacgggcaccaccc

SEQ ID NO: 1200 - RNA, 20 nt, Artificial Sequence
  Description of Artificial Sequence Synthetic oligonucleotide
gaucaggaugggcaccaccc

SEQ ID NO: 1201 - RNA, 20 nt, Artificial Sequence
  Description of Artificial Sequence Synthetic oligonucleotide
gaccaguaugggcaccaccc

SEQ ID NO: 1202 - RNA, 20 nt, Artificial Sequence
  Description of Artificial Sequence Synthetic oligonucleotide
gaccaagaugggcaccaccc

SEQ ID NO: 1203 - RNA, 20 nt, Artificial Sequence
  Description of Artificial Sequence Synthetic oligonucleotide
gaccaugaugggcaccaccc

SEQ ID NO: 1204 - RNA, 20 nt, Artificial Sequence
  Description of Artificial Sequence Synthetic oligonucleotide
gaccaggaucggcaccaccc

SEQ ID NO: 1205 - RNA, 20 nt, Artificial Sequence
  Description of Artificial Sequence Synthetic oligonucleotide
gaccaggaugggcaccacac

SEQ ID NO: 1206 - RNA, 20 nt, Artificial Sequence
  Description of Artificial Sequence Synthetic oligonucleotide
gaccaggaugggcaccgccc

SEQ ID NO: 1207 - RNA, 20 nt, Artificial Sequence
  Description of Artificial Sequence Synthetic oligonucleotide
gaccaggaugggaaccaccc

SEQ ID NO: 1208 - RNA, 20 nt, Artificial Sequence
  Description of Artificial Sequence Synthetic oligonucleotide
gaccaggaugggcacgaccc

SEQ ID NO: 1209 - RNA, 20 nt, Artificial Sequence
  Description of Artificial Sequence Synthetic oligonucleotide
gaccaggaugggcacuaccc

SEQ ID NO: 1210 - RNA, 20 nt, Artificial Sequence
  Description of Artificial Sequence Synthetic oligonucleotide
gaccaggaugggccccaccc

SEQ ID NO: 1211 - RNA, 20 nt, Artificial Sequence
  Description of Artificial Sequence Synthetic oligonucleotide
gaccaggaugggcaccagcc

SEQ ID NO: 1212 - RNA, 20 nt, Artificial Sequence
  Description of Artificial Sequence Synthetic oligonucleotide
gaacgacuaguuaggcgugu

SEQ ID NO: 1213 - DNA, 22 bp, Artificial Sequence
  Description of Artificial Sequence Synthetic oligonucleotide
ccggggtggtgcccatcctggt

SEQ ID NO: 1214 - RNA, 106 nt, Artificial Sequence
  Description of Artificial Sequence Synthetic polynucleotide
  modified_base (1)..(20): a, c, u, g, unknown or other
nnnnnnnnnnnnnnnnnnnnguuuaagagcuaagcuggaaacagcauagcaaguuuaaauaaggcuaguccguuaucaacuugaaaaaguggcaccgagucggugc

(19) United States
(12) Patent Application Publication
(10) Pub. No.: US 2022/0259593 A1
(43) Pub. Date: Aug. 18, 2022

(54) CONTROL OF MAMMALIAN GENE DOSAGE USING CRISPR

(71) Applicant: The Regents of the University of California, Oakland, CA (US)

(72) Inventors: Jonathan Weissman, San Francisco, CA (US); Marco Jost, Berkeley, CA (US); Maximilian A. Horlbeck, Boston, MA (US); Daniel Santos, San Francisco, CA (US); Reuben Saunders, San Francisco, CA (US)

(73) Assignee: The Regents of the University of California, Oakland, CA (US)

(21) Appl. No.: 17/584,176

(22) Filed: Jan. 25, 2022

Related U.S. Application Data
(63) Continuation of application No. PCT/US2020/043608, filed on Jul. 24, 2020.
(60) Provisional application No. 62/879,348, filed on Jul. 26, 2019.

(51) Int. Cl. C12N 15/113; C12N 15/11; C12N 9/22; C12N 15/85

(52) U.S. Cl. CPC C12N 15/113; C12N 15/111; C12N 2310/14; C12N 15/85; C12N 2310/20; C12N 9/22

(57) ABSTRACT

The present disclosure provides methods and compositions for precisely controlling the expression levels of mammalian genes using CRISPRi or CRISPRa and one or more modified sgRNAs. The methods and compositions are useful for, inter alia, titrating the expression of a gene of interest, identifying drug targets and mechanisms of drug resistance, and enabling the analysis of and control over metabolic and signaling pathway fluxes.

### CROSS REFERENCE TO RELATED APPLICATIONS

The present application is a continuation of International Application No. PCT/US20/43608, filed Jul. 24, 2020, which claims priority to U.S. Provisional Pat. Appl. No. 62/879,348, filed on Jul. 26, 2019, wherein each application is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under grant nos. HG009490 and R01 DA036858 awarded by The National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 8, 2020, is named 081906-1204127-236610WO_SL.txt and is 367,829 bytes in size.

### BACKGROUND OF THE INVENTION

The complexity of biological processes arises not only from the set of expressed genes but also from quantitative differences in their expression levels. As a classic example, some genes are haploinsufficient and thus are sensitive to a 50% decrease in expression, whereas other genes are permissive to far stronger depletion (1). Enabled by tools to titrate gene expression levels such as series of promoters or hypomorphic mutants, the underlying expression-phenotype relationships have been explored systematically in yeast (2-4) and bacteria (5-8). These efforts have revealed gene- and environment-specific effects of changes in expression levels (4) and yielded insight into the opposing evolutionary forces that determine gene expression levels, including the cost of protein synthesis and the need for robustness against random fluctuations (3,6,8). The availability of equivalent tools in mammalian systems would enable similar efforts to understand these forces in more complex models as well as additional applications.

The discovery and development of artificial transcription factors, such as TALEs (10) or the CRISPR-based effectors underlying CRISPR interference (CRISPRi) and activation (CRISPRa) (11), has brought tools to precisely modify genomic sequences and systematically control gene expression in all cell types, including mammals.

There remains a need, however, for methods allowing the precise and predictable control of the expression levels of genes, including mammalian genes, to desired target levels. The present disclosure satisfies this need and provides other advantages as well.

### BRIEF SUMMARY OF THE INVENTION

In one aspect, the present disclosure provides a method of generating a set of single guide RNAs (sgRNAs) capable of driving a series of discrete expression levels of a target gene in a cell population using CRISPR interference (CRISPRi) or CRISPR activation (CRISPRa), the method comprising: (i) providing a first sgRNA that targets the gene, wherein the last 19 nucleotides of the targeting sequence of the first sgRNA are 100% homologous to the target DNA sequence; (ii) providing a second sgRNA that targets the gene, wherein the last 19 nucleotides of the targeting sequence of the second sgRNA comprises one or more mismatches with the target DNA sequence such that the CRISPRi or CRISPRa activity on the gene obtained using the second sgRNA is intermediate between that obtained using the first sgRNA and that obtained using a scrambled sgRNA providing no CRISPRi or CRISPRa activity on the gene; and (iii) providing a third sgRNA that targets the gene, wherein the last 19 nucleotides of the targeting sequence of the third sgRNA comprises one or more mismatches with the target DNA sequence such that the CRISPRi or CRISPRa activity on the gene obtained using the third sgRNA is intermediate between that obtained using the second sgRNA and that obtained using a scrambled sgRNA providing no CRISPRi or CRISPRa activity on the gene; wherein the mismatches of the second and third sgRNAs are selected according to the following rules: (a) the CRISPRi or CRISPRa activity of the second sgRNA is designed to be greater than that of the third sgRNA based on the following positional relationships, wherein the positions correspond to the number of bases in the sgRNAs upstream from the sgRNA PAM: −19>−18>−17>−16−15−14>−13>−12>−11>−10>−9>−8>−4>−7−6−5−3−2−1; or (b) the CRISPRi or CRISPRa activity of the second sgRNA is designed to be greater than that of the third sgRNA based on the following base pair rankings of the mismatched nucleotides, wherein the first nucleotide in each pair corresponds to the ribonucleotide within the sgRNA and the second nucleotide corresponds to the deoxyribonucleotide within the target DNA: rG:dT>rU:dG>rG:dA rG:dG>rC:dA>rU:dT>rA:dA>rC:dT>rA:dC>rA:dG>rU:dC rC:dC.

In some embodiments, the method further comprises providing one or more additional sgRNAs, wherein the last 19 nucleotides of the targeting sequence of each of the one or more additional sgRNAs comprise at least one mismatch with the target DNA sequence, wherein each of the one or more additional sgRNAs provide CRISPRi or CRISPRa activity on the gene that is intermediate between that obtained using the third sgRNA and that obtained using a scrambled sgRNA providing no CRISPRi or CRISPRa activity on the gene, and wherein the mismatches with the template DNA of each of the one or more additional sgRNAs are selected according to rules (a) and (b) above. In some embodiments, the target gene is a mammalian gene. In some embodiments, the mammalian gene is a human gene.

In another aspect, the present disclosure provides a set of single guide RNAs (sgRNAs) for obtaining a series of discrete expression levels of a target gene using CRISPRi or CRISPRa, comprising: (i) a first sgRNA that targets the gene, wherein the last 19 nucleotides of the targeting sequence of the first sgRNA is 100% homologous to the target DNA sequence; (ii) a second sgRNA that targets the gene, wherein the last 19 nucleotides of the targeting sequence of the second sgRNA comprises one or more mismatches with the target DNA sequence such that the CRISPRi or CRISPRa activity on the gene obtained using the second sgRNA is intermediate between that obtained using the first sgRNA and that obtained using a scrambled sgRNA providing no CRISPRi or CRISPRa activity on the gene; and (iii) a third sgRNA that targets the gene, wherein the last 19 nucleotides of the targeting sequence of the third sgRNA comprises one or more mismatches with the target DNA sequence such that the CRISPRi or CRISPRa activity obtained using the third sgRNA is intermediate between that obtained using the second sgRNA and that obtained using a scrambled sgRNA providing no CRISPRi or CRISPRa activity on the gene; wherein the mismatches of the second and third sgRNAs are selected according to the following rules: (a) the CRISPRi or CRISPRa activity of the second sgRNA is designed to be greater than that of the third sgRNA based on the following positional relationships, wherein the positions correspond to the number of bases in the sgRNAs upstream from the sgRNA PAM: −19>−18 >−17>−16≈−15≈−14>−13>−12>−11>−10>−9>−8>−4>−7≈−6≈−5≈−3≈−2≈−1; or (b) the CRISPRi or CRISPRa activity of the second sgRNA is designed to be greater than that of the third sgRNA based on the following base pair rankings of the mismatched nucleotides, wherein the first nucleotide in each pair corresponds to the ribonucleotide within the sgRNA and the second nucleotide corresponds to the deoxyribonucleotide within the target DNA: rG:dT>rU:dG>rG:dA rG:dG>rC:dA>rU:dT>rA:dA>rC:dT>rA:dC>rA:dG>rU:dC≈rC:dC.

In some embodiments, the set of sgRNAs further comprises one or more additional sgRNAs, wherein the last 19 nucleotides of the targeting sequences of each of the one or more additional sgRNAs comprise at least one mismatch with the target DNA sequence, wherein each of the one or more additional sgRNAs provide CRISPRi or CRISPRa activity on the gene that is intermediate between that obtained using the third sgRNA and a scrambled sgRNA providing no CRISPRi or CRISPRa activity on the gene, and wherein the CRISPRi or CRISPRa activity of each of the one or more additional sgRNAs on the gene is determined according to rules (a) and (b) above.

In some embodiments, the set comprises 2, 3, 4, 5, 6, 7, 8, 9, 10, 11, 12, 13, 14, 15, 16, 17, 18, 19, 20 or more sgRNAs providing intermediate levels of CRISPRi or CRISPRa activity on the gene between that obtained using the first sgRNA and that obtained using a scrambled sgRNA providing no CRISPRi or CRISPRa activity on the gene.

In another aspect, the present disclosure provides a method of obtaining a series of discrete expression levels of a target gene in a plurality of cells, the method comprising: contacting the plurality of cells with the set of any of the herein-disclosed sgRNAs; and contacting the plurality of cells with a nuclease-deficient sgRNA-mediated nuclease (dCas9), wherein the dCas9 comprises a dCas9 domain fused to a transcriptional modulator; thereby generating a plurality of test cells, wherein each test cell comprises an sgRNA and the dCas9, wherein the sgRNA present in a given test cell guides the dCas9 in the test cell to the target gene and modulates its expression level as a function of the absence or presence of one or more mismatches with the target DNA sequence according to rules (a) and (b) above.

In some embodiments, the transcriptional modulator is a transcriptional repressor. In some embodiments, the transcriptional repressor is KRAB. In some embodiments, the transcriptional modulator is a transcriptional activator. In some embodiments, the transcriptional activator is VP64. In some embodiments, the cells are mammalian cells. In some embodiments, the cells are human cells. In some embodiments, each sgRNA is encoded by an expression cassette comprising a polynucleotide encoding the sgRNA, operably linked to a promoter. In some embodiments, the dCas9 is encoded by an expression cassette comprising a polynucleotide encoding the dCas9, operably linked to a promoter.

In some embodiments, the method further comprises determining the relationship between the expression level of the target gene and a phenotype, comprising: (i) determining the identity of the sgRNA present in a given test cell; (ii) assessing the phenotype of the test cell; and (iii) correlating the expression level of the gene targeted by the sgRNA identified in step (i) and the phenotype assessed in step (ii).

In some embodiments, assessing the phenotype of the cells comprises fluorescence activated cell sorting, affinity purification of the cells, measuring the transcriptomes of the cells, or measuring the growth, proliferation, and/or survival of the cells. In some embodiments, the transcriptomes of the cells are measured by perturb-seq.

In another aspect, the present disclosure provides a method of determining a therapeutic window for the inhibition of a gene, the method comprising determining the relationship between the expression level of the gene and the phenotype according to any of the herein-described methods for a plurality of sgRNAs targeting the gene, wherein the transcriptional modulator is a transcriptional repressor, and wherein the phenotype of the cells is assessed by measuring cell growth or survival; and further comprising: (iv) determining the minimum level of expression of the gene that is compatible with cell growth or survival, thereby determining the lower boundary of the therapeutic window for the inhibition of the gene.

In another aspect, the present disclosure provides a library of single guide RNAs (sgRNAs) for obtaining a series of discrete expression levels of a plurality of genes in a cell population, comprising any of the herein-described sets of sgRNAs according for each of the plurality of genes.

In some embodiments, the plurality of genes comprises 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, or more genes. In some embodiments, the library contains at least 1000, 5000, 10,000, 20,000, 30,000, 40,000, 50,000, 60,000, 70,000, 80,000, 90,000, or 100,000 structurally distinct sgRNAs.

In another aspect, the present disclosure provides a method of obtaining a series of expression levels of a plurality of genes in a cell population, the method comprising: contacting the cell population with any one of the herein-disclosed sgRNA libraries; and contacting the cell population with a nuclease-deficient sgRNA-mediated nuclease (dCas9), wherein the dCas9 comprises a dCas9 domain fused to a transcriptional modulator; thereby generating a population of test cells, wherein each test cell within the population comprises an sgRNA targeting one of the plurality of genes and the dCas9; and wherein the sgRNA present in a given test cell guides the dCas9 in the test cell to the target gene of the sgRNA and modulates its expression level as a function of the absence or presence of one or more mismatches with the target DNA sequence according to rules (a) and (b) above.

In some embodiments, the transcriptional modulator is a transcriptional repressor. In some embodiments, the transcriptional repressor is KRAB. In some embodiments, the transcriptional modulator is a transcriptional activator. In some embodiments, the transcriptional activator is VP64. In some embodiments, each sgRNA within the library is encoded by an expression cassette comprising a polynucleotide encoding the sgRNA, operably linked to a promoter. In some embodiments, the dCas9 is encoded by an expression cassette comprising a polynucleotide encoding the dCas9, operably linked to a promoter.

In some embodiments, the method further comprises determining the relationship between the expression level of any one of the plurality of genes and a phenotype, comprising: (i) determining the identity of the sgRNA expressed in a given test cell within the population; (ii) assessing the phenotype of the test cell; and (iii) correlating the expression level of the target gene associated with the identified sgRNA and the assessed phenotype of the test cell.

In some embodiments, assessing the phenotype of the cells comprises fluorescence activated cell sorting, affinity purification of the cells, measuring the transcriptomes of the cells, or measuring the growth, proliferation, and/or survival of the cells. In some embodiments, the transcriptomes of the cells are measured by perturb-seq.

In another aspect, the present disclosure provides a method of identifying a gene target of a cytotoxic agent or a drug candidate, the method comprising: (i) generating a population of test cells according to any one of the herein-disclosed methods; (ii) contacting the population of test cells with a sub-lethal or sub-therapeutic amount of the cytotoxic agent or drug candidate; (iii) identifying test cells within the population that display a phenotype in the presence of the sub-lethal or sub-therapeutic amount of the cytotoxic agent or drug candidate that is not displayed by cells in the presence of the sub-lethal or sub-therapeutic amount of the cytotoxic agent or drug candidate but in the absence of the dCas9 or of an sgRNA; (iv) determining the identity of the sgRNAs present within the test cells displaying the phenotype; and (v) identifying genes that are targeted by one or more distinct sgRNAs identified in step (iv); wherein a gene that displays the phenotype at one or more levels of expression resulting from the presence of one or more distinct sgRNAs represents a candidate gene target of the cytotoxic agent or drug candidate.

In some embodiments, at least one of the sgRNAs targeting the candidate gene target comprises a mismatch with the target DNA in the last 19 nucleotides of its targeting sequence. In some embodiments, the at least one sgRNA provides a level of CRISPRi or CRISPRa activity on the gene that is less than 50% of the level obtained using an sgRNA comprising 100% homology in the last 19 nucleotides of its targeting sequence to the target DNA sequence.

### BRIEF DESCRIPTION OF THE DRAWINGS

The present application includes the following figures. The figures are intended to illustrate certain embodiments and/or features of the compositions and methods, and to supplement any description(s) of the compositions and methods. The figure does not limit the scope of the compositions and methods, unless the written description expressly indicates that such is the case.

FIGS. 1A-1C. Mismatched sgRNAs titrate GFP expression at the single-cell level. (FIG. 1A) Experimental design to test knockdown conferred by all mismatched variants of a GFP-targeting sgRNA. (FIG. 1B) Distributions of GFP levels in cells with perfectly matched sgRNA (top), mismatched sgRNAs (middle), and non-targeting control sgRNA (bottom). Sequences of sgRNAs are indicated on the right (without the PAM). Figure discloses SEQ ID NOS 1196-1212, respectively, in order of appearance. (FIG. 1C) Relative activities of all mismatched sgRNAs, defined as the ratio of fold-knockdown conferred by a mismatched sgRNA to fold-knockdown conferred by the perfectly matched sgRNA. Relative activities are displayed as the mean of two biological replicates. Figure discloses SEQ ID NO: 1213.

FIGS. 2A-2B. Details of the GFP mismatch experiment. (FIG. 2A) Comparison of relative activities measured in two biological replicates. Relative activity was defined as the fold-knockdown of each mismatched variant (GFPsgRNA[non-targeting]/GFPsgRNA[variant]) divided by the fold-knockdown of the perfectly-matched sgRNA. The background fluorescence of a GFP-strain was subtracted from all GFP values prior to other calculations. (FIG. 2B) KDE plots of GFP distributions 10 days after transducing K562 GFP+ cells with the perfectly-matched sgRNA, a non-targeting sgRNA, and each of the 57 singly-mismatched variants. Fluorescence of a GFP− K562 strain is shown in gray. Although most GFP distributions are unimodal, some are broadened compared to those with the perfectly matched sgRNA or the negative control sgRNA. This heterogeneity could be a consequence of the random integration of the GFP locus, cell-to-cell differences in expression of the dCas9-KRAB effector in our polyclonal cell line, the amplification of gene expression bursts by long GFP half-lives, or a combination of these factors.

FIGS. 3A-3G. A large-scale CRISPRi screen identifies factors governing mismatched sgRNA activity. (FIG. 3A) Design of large-scale mismatched sgRNA library. (FIG. 3B) Schematic of pooled CRISPRi screen to determine activities of mismatched-sgRNAs. (FIG. 3C) Growth phenotypes (γ) in K562 and Jurkat cells for four sgRNA series, with the perfectly matched sgRNAs shown in darker colors and mismatched sgRNAs shown in corresponding lighter colors. Phenotypes represent the mean of two biological replicates. Differences in absolute phenotypes likely reflect cell type-specific essentiality. (FIG. 3D) Comparison of mismatched sgRNA relative activities in K562 and Jurkat cells. Marginal histograms depict distributions of relative activities along the corresponding axes. (FIG. 3E) Distribution of mismatched sgRNA relative activities stratified by position of the mismatch. Position −1 is closest to the PAM. (FIG. 3F) Distribution of mismatched sgRNA relative activities stratified by type of mismatch, grouped by mismatches located in positions −19 to −13 (PAM-distal region), positions −12 to −9 (intermediate region), and positions −8 to −1 (PAM-proximal/seed region). Division into these regions was based on previous work (12,13) and the patterns in FIG. 3E. (FIG. 3G) Comparison of mean apparent on-rates measured in vitro for mismatched variants of a single sgRNA (22) and mean relative activities from large-scale screen. Values are compared for identical combinations of mismatch type and mismatch position; mean relative activities were calculated by averaging relative activities for all mismatched sgRNAs with a given combination.

FIGS. 4A-4H. Additional analysis of large-scale mismatched sgRNA screen. (FIGS. 4A-4B) Comparison of growth phenotypes (γ) of all sgRNAs derived from biological replicates of the (FIG. 4A) K562 and (FIG. 4B) Jurkat screens. (FIG. 4C) Comparison of growth phenotypes (γ) of perfectly matched sgRNAs from the K562 screen in this work and a previously published K562 screen (19) (average of two biological replicates). (FIG. 4D) Comparison of growth phenotypes (γ) of perfectly matched sgRNAs in K562 and Jurkat cells reveals substantial differences, likely reflecting cell-type specific gene essentiality (average of two biological replicates). (FIG. 4E) Distribution of mismatched sgRNA relative activities for sgRNAs with 1 mismatch (left) or 2 mismatches (right). (FIG. 4F) Distribution of mismatched sgRNA relative activities stratified by sgRNA GC content, grouped by mismatches located in positions −19 to −13 (PAM-distal region), positions −12 to −9 (intermediate region), and positions −8 to −1 (PAM-proximal/seed region). (FIG. 4G) Distribution of mismatched sgRNA relative activities stratified by the identity of the 2 bases flanking the mismatch, grouped by mismatches located in positions −19 to −13 (PAM-distal region), positions −12 to −9 (intermediate region), and positions −8 to −1 (PAM-proximal/seed region). (FIG. 4H) Distribution of sgRNA series by number of sgRNAs with intermediate activity (0.1<relative activity <0.9), using only sgRNAs with a single mismatch (top) or all mismatched sgRNAs (bottom).

FIGS. 5A-5G. Identification and characterization of intermediate-activity constant regions. (FIG. 5A) Design of constant region variant library. (FIG. 5B) Mean relative activities of constant region variants, calculated by averaging relative activities for all targeting sequences. Gray margins denote 95% confidence interval. Inset: Focus on 6 constant region variants with higher activity than the original constant region. Black diamonds denote mean relative activity, gray dots relative activities with individual targeting sequences. (FIG. 5C) Mapping of constant region variant relative activities onto constant region structure. Each constant region base is colored by the average relative activity of the three single constant region variants carrying a single mutation at that position. Positions mutated in 6 highly active constant regions (inset in FIG. 5B) are indicated by colored dots. The BlpI site (gray) is used for cloning and was not mutated. Figure discloses SEQ ID NO: 1214. (FIG. 5D) Constant region activities by targeting sequence, plotted against ranked mean constant region activity. For each gene, the activities with the strongest targeting sequence are shown as rolling means with a window size of 50. (FIGS. 5E-5G) Constant region activities by targeting sequence for all three targeting sequences against the indicated genes. Growth phenotypes (γ) of each targeting sequence paired with the unmodified constant region are indicated in the legend.

FIGS. 6A-6E. Additional analysis of modified constant regions. (FIG. 6A) Comparison of growth phenotypes measured in each biological replicate after 4, 6, or 8 days of growth from t0. Data from Day 4 was used for all subsequent analyses. (FIG. 6B) Comparison of relative % knockdown (quantified via RT-qPCR) and mean relative growth phenotype for 10 intermediate-activity constant region variants paired with two targeting sequences against DPH2. (FIG. 6C) Relative activities of constant regions paired with all 30 targeting sequences, ranked by the average strength of each constant region and displayed as rolling means with a window size of 50. (FIG. 6D) Distribution of all pairwise correlations of constant region relative activities within and between gene targets. N.S.; no significant difference according to two-tailed Student's t-test (p>0.05). (FIG. 6E) Relative activity of each indicated target sequence:constant region pair vs. the mean relative activity of the respective constant region for all targets. Growth phenotypes (γ) with the unmodified constant region are indicated in the figure legends. Lines represent rolling means of individual data points.

FIGS. 7A-7D. Neural network predictions of sgRNA activity. (FIG. 7A) Schematic of a singly-mismatched sgRNA feature array (Xi) and the convolutional neural network architecture trained on pairs of such arrays and their corresponding relative activities (yi). Black squares in Xi represent the value 1 (the presence of a base at the indicated position); white represents 0. The mean prediction from 20 independently trained models was used to assign a final prediction (ŷ) to each sgRNA in the hold-out validation set. (FIG. 7B) Comparison of measured relative growth phenotypes from the large-scale screen and predicted activities assigned by the neural network. Marginal histograms show distributions of relative activities along the corresponding axes. (FIG. 7C) Distribution of Pearson r values (predicted vs. measured relative activity) for each sgRNA series in the validation set. (FIG. 7D) Comparison of measured relative activity (i.e., relative knockdown) in the GFP experiment and predicted relative sgRNA activity. Two outliers with lower-than-predicted activity are annotated with their respective mismatch position and type. Predictions are shown as mean±S.D. from the 20-model ensemble.

FIGS. 8A-8I. Additional details for the neural network. (FIG. 8A) Graph of the CNN model architecture. (FIG. 8B) Model loss, measured as root mean squared error, for training and validation data over 25 training epochs. Each line represents one of 20 models trained. The final models used for our predictions were only trained for 8 epochs, as additional cycles only reduced training loss without significant improvement in validation loss (i.e., the model becomes over-fit). (FIG. 8C) Explained variance (r2) of validation sgRNA relative activities for each individual model (black), and for the mean prediction of all 20 models (red). (FIG. 8D) Validation error stratified by mismatch position. (FIG. 8E) Validation error stratified by mismatch type. (FIG. 8F) Partitioning of sgRNAs into bins based on relative activity in the large-scale K562 screen. (FIG. 8G) Confusion matrix showing the fraction of sgRNAs in each actual (measured) activity bin that were assigned to each predicted bin by the CNN model. Each row sums to 1. (FIG. 8H) Statistics indicating the requisite number of randomly sampled sgRNAs from each activity bin to have a given probability of selecting at least one sgRNA with true activity in that bin. Simulations are based on the probabilities outlined in the confusion matrix (FIG. 8E). (FIG. 8I) Similar to FIG. 8H, with random sampling from any of the intermediate activity bins (1-3) to yield at least one sgRNA with intermediate activity (0.1-0.9).

FIGS. 9A-9F. Additional details for the linear model. (FIG. 9A) Comparison of measured relative growth phenotypes from the large-scale screen and predicted activities assigned by the elastic net linear model. Marginal histograms show distributions of relative activities along the corresponding axes. (FIG. 9B) Comparison of measured relative activity (relative knockdown) in the GFP experiment and predicted relative sgRNA activity. (FIG. 9C) Comparison of predicted relative activities from the linear model and the neural network, based on the validation set of singly-mismatched sgRNAs. (FIG. 9D) Regression coefficients assigned to each feature in the linear model. 228 features (gray, blue) describe the position and type of mismatch; 42 features (gold) carry other information about the sgRNA and genomic context surrounding the protospacer. These features are detailed in subsequent panels. (FIG. 9E) Linear coefficients for features of the sgRNA and targeted locus. TSS; transcription start site. (FIG. 9F) Linear coefficients for features covering positions in the distal, intermediate, and seed regions of the targeting sequence (highlighted blue in FIG. 9D).

FIGS. 10A-10C. Compact mismatched sgRNA library targeting essential genes. (FIG. 10A) Design of library. For activity bins lacking a previously measured sgRNA, novel mismatched sgRNAs were included according to predicted activity. (FIG. 10B) Distribution of relative activities from the large-scale library (gray) and the compact library (purple) in K562 cells. (FIG. 10C) Comparison of relative activities of mismatched sgRNAs in HeLa and K562 cells. Marginal histograms show the distributions of relative activities along the corresponding axes.

FIGS. 11A-11K. Additional analysis of the compact allelic series screen. (FIG. 11A) Composition of the compact library, in terms of previously measured relative activities in the large-scale screen (dark purple), or predicted relative activities assigned by the CNN model ensemble (light purple). Perfectly matched sgRNAs, which by definition have relative activities of 1.0, comprise 20% of the library but were not included in the histogram. (FIG. 11B) Distribution of mismatch positions and types for singly-mismatched sgRNAs in the compact library, for previously measured (dark purple) and CNN-imputed (light purple) sgRNAs. (FIG. 11C) Heatmap showing the distribution of mutated positions for doubly-mismatched sgRNAs in the compact library. (FIG. 11D) Comparison of growth phenotypes measured in each K562 biological replicate 4- and 7-days post-transduction. Data from Day 7 was used for all subsequent analyses. (FIG. 11E) Comparison of growth phenotypes measured in each HeLa biological replicate 6- and 8-days post-transduction. Data from Day 8 was used for all subsequent analyses. (FIG. 11F) Comparison of growth phenotypes in HeLa and K562 cells (γ expressed as the average of biological replicate measurements). (FIG. 11G) Measured vs. predicted relative activities of CNN-imputed sgRNAs in K562 cells (left) and HeLa cells (right). A small number of points beyond the y-axis limits were excluded to more clearly display the bulk of the distribution. n=6,147 sgRNAs; r2=squared Pearson correlation coefficient. (FIG. 11H) Comparison of sgRNA composition and model error for the large-scale and compact libraries. The CNN-imputed guides had substantially higher predicted activities than those for the large-scale validation set; higher predicted activity was generally associated with higher model error for the validation (red) and imputed (blue) sgRNA sets, consistent with the discrepancy in model performance on each set. (FIG. 11I) Distribution of the number of intermediate-activity mismatched sgRNAs targeting each gene in the compact library. The number of genes with at least 2 intermediate activity sgRNAs is indicated above each histogram; sgRNA activities were quantified for 1907 and 1442 genes in K562 and HeLa cells, respectively. Note that here activities are aggregated by gene as opposed to by series, as was done in FIG. 4I. (FIG. 11J) Comparison of phenotypes measured in each biological replicate after 12 days of growth in the drug screen. (FIG. 11K) Comparison of vehicle- (γ) and lovastatin-treatment (τ) growth phenotypes for all sgRNAs in the compact library. Knockdown of HMG-CoA reductase (HMGCR) greatly sensitizes cells to lovastatin, compared to knockdown of other genes such as tubulin (TUBB).

FIGS. 12A-12E. Summary of Perturb-seq experiment. (FIG. 12A) Schematic of Perturb-seq strategy to capture single-cell transcriptomes with matched sgRNA identities. (FIG. 12B) Summary of sequencing and perturbation assignment statistics. (FIG. 12C) Distribution of number of cells captured per perturbation. Median: 122 cells per perturbation; 5th to 95th percentile: 66-277 cells per perturbation. (FIGS. 12D-12E) Comparison of (FIG. 12D) growth phenotypes (γ) and (FIG. 12E) relative activities measured in the large-scale mismatched sgRNA screen and in the Perturb-seq experiment. Differences are likely due to the different timescales and the different vectors used.

FIGS. 13A-13B. Target gene expression in cells with indicated perturbations. (FIG. 13A) Distribution of target gene expression levels, quantified as target gene UMI count normalized to total UMI count per cell. (FIG. 13B) Mean target gene expression levels for target genes with low basal expression levels.

FIG. 14. Distributions of target gene expression in cells with indicated perturbations. Expression is quantified as raw target gene UMI count.

FIGS. 15A-15J. Rich phenotyping of cells with intermediate-activity sgRNAs by Perturb-seq. (FIG. 15A) Distributions of HSPA9 and RPL9 expression in cells with indicated perturbations. Expression is quantified as target gene UMI count normalized to total UMI count per cell. sgRNA activity is calculated using relative γ measurements from the Perturb-seq cell pool after 5 days of growth. (FIG. 15B) Distributions of total UMI counts in cells with indicated perturbations. (FIG. 15C) Comparison of median UMI count per cell and target gene expression in cells with GATA1- or POLR2H-targeting sgRNAs. (FIG. 15D) Right: Expression profiles of 100 genes in populations with HSPA9-targeting sgRNAs. Genes were selected by lowest FDR-corrected p-values in cells with the strongest sgRNA from a two-sided Kolmogorov-Smirnov test (Methods). Expression is quantified as z-score relative to population of cells with non-targeting sgRNAs. Left: Growth phenotype and knockdown for each sgRNA. (FIG. 15E) Distribution of gene expression changes in populations with indicated sgRNAs. Magnitude of gene expression change is calculated as sum of z-scores of genes differentially expressed in the series (FDR-corrected p<0.05 with any sgRNA in the series, two-sided Kolmogorov-Smirnov test, Methods), with z-scores of individual genes signed by the direction of change in cells with the perfectly matched sgRNA. Distribution for negative control sgRNAs is centered around 0 (dashed line). (FIG. 15F) Comparison of relative growth phenotype and magnitude of gene expression change for all individual sgRNAs. Growth phenotype and magnitude of gene expression change are normalized in each series to those of the sgRNA with the strongest knockdown. Individual series highlighted as indicated. (FIG. 15G) Comparison of magnitude of gene expression and target gene knockdown, as in FIG. 15F. (FIG. 15H) UMAP projection of all single cells with assigned sgRNA identity in the experiment, colored by targeted gene. Clusters clearly assignable to a genetic perturbation are labeled. Cluster labeled * contains a small number of cells with residual stress response activation and could represent apoptotic cells. Note that ˜5% cells appear to have confidently but wrongly assigned sgRNA identities, as evident within the cluster of cells with HSPAS knockdown (Methods). Given the strong trends in the other results, we concluded that such misassignment did not substantially affect our ability to identify trends within cell populations and in the future may be avoided by approaches to directly capture the expressed sgRNA34. (FIG. 15I) UMAP projection, as in FIG. 15H, with selected series colored by sgRNA activity. (FIG. 15J) Comparison of extent of ISR activation to ATP5E UMI count in cells with knockdown of ATP5E or control cells.

FIGS. 16A-16I. Phenotypes resulting from target gene titration. (FIG. 16A) Distributions of total UMI counts in cells with the perfectly matched sgRNA against the indicated genes. (FIG. 16B) Left: Comparison of median UMI count per cell and relative growth phenotype in cells with sgRNAs targeting BCR, GATA1, or POLR2H or control cells. Right: Comparison of median UMI count per cell and target gene expression. (FIG. 16C) Cell cycle scores (Methods) for populations of cells with individual sgRNAs. (FIG. 16D) Magnitudes of gene expression change of populations with perfectly matched sgRNAs targeting indicated genes. Magnitude of gene expression change is calculated as sum of z-scores of genes differentially expressed in the series (FDR-corrected p<0.05 with any sgRNA in the series, two-sided Kolmogorov-Smirnov test, Methods), with z-scores of each gene in individual cells signed by the average direction of change in the population. (FIG. 16E) Comparison of magnitude of gene expression change to growth phenotype (γ) for all perfectly matched sgRNAs in the experiment. (FIG. 16F) Comparison of relative growth phenotype and magnitude of gene expression change for all individual sgRNAs, as in FIG. 15F but without increased transparency for individual series. (FIG. 16G) Comparison of magnitude of gene expression and target gene knockdown, as in FIG. 15G but without increased transparency for individual series. (FIG. 16H) Comparison of relative growth phenotype and target gene expression, as in FIG. 15F. (FIG. 16I) Comparison of measured growth phenotype (γ, not normalized to strongest sgRNA) and target gene expression, as in FIG. 15F.

FIGS. 17A-17B. Diverse phenotypes resulting from essential gene depletion. (FIG. 17A) Clustered correlation heatmap of perturbations. Gene expression profiles for genes with mean UMI count >0.25 in the entire population were z-normalized to expression values in cells with negative control sgRNAs and then averaged for populations with the same sgRNA. Crosswise Pearson correlations of all averaged transcriptomes were clustered by the Ward variance minimization algorithm implemented in scipy. (FIG. 17A B) UMAP projection, distribution of cells with indicated sgRNAs, target gene expression (rolling mean over 50 cells), and magnitudes of transcriptional changes for all differentially expressed genes and selected ISR regulon genes (rolling mean over 50 cells) for cells with knockdown of ATP5E or control cells.

### DETAILED DESCRIPTION OF THE INVENTION

### 1. Introduction

The present disclosure provides compositions and methods to precisely and predictably control the expression levels of mammalian genes to desired target levels. Methods and compositions are provided to systematically control the activity, e.g., by modulating the residence time, of a fusion protein of a transcriptional modulator, e.g., a transcription factor and nuclease-dead Cas9 (dCas9) at a gene of interest, thereby downregulating or upregulating the expression of the gene depending, e.g., on the residence time. Using the present methods and compositions, it is possible to regulate the expression of endogenous genes by varying degrees to levels between, e.g., 1% and 5000% of the normal expression level. These methods, inter alia, enable the titration of the expression of a gene of interest, allow for systematic mapping of gene dose-response curves, facilitate identification of drug targets and mechanisms of drug resistance, and enable analysis of and afford control over metabolic and signaling pathway fluxes.

The present methods extend previously developed CRISPR-based transcriptional repression (CRISPR interference, or CRISPRi) and overexpression (CRISPR activation, or CRISPRa), in which dCas9 is fused to a transcriptional repressor or activator, respectively, and is targeted to endogenous genes via a single guide RNA (sgRNA). The dCas9-sgRNA complex binds to DNA loci via basepairing between the sgRNA and DNA, i.e., the targeting sequence of the sgRNA and the target DNA sequence on the template DNA, and the fused transcriptional repressor or activator leads to downregulation or upregulation of the gene, respectively. The present disclosure provides methods to control the activity of dCas9 at a given DNA locus, e.g., by introducing mismatches into the sgRNA (e.g., within the targeting sequence of the sgRNA) or by introducing mutations into the sgRNA constant region. Without being bound by the following theory, it is believed that these modifications reduce the extent of transcriptional downregulation or upregulation by CRISPRi or CRISPRa, respectively, by reducing the residence time of dCas9 on the target DNA. The extent of transcriptional downregulation or upregulation can be varied systematically, thus affording precise control over expression levels of the target gene.

The present disclosure also provides sets of sgRNAs targeting individual genes, or targeting individual DNA sites within genes, allowing the generation of series of discrete expression levels of the genes, as well as libraries comprising a plurality of sgRNA sets and thereby allowing the generation of series of discrete expression levels for each of a multitude of genes, including libraries targeting up to all or virtually all of the genes in a genome. In such embodiments, each sgRNA within the set or library is selected to generate a discrete amount of transcriptional repression or activation of the targeted gene or genes by CRISPRi or CRISPRa, respectively.

The present disclosure also provides rules, factors, and parameters to determine how a given mismatch in an sgRNA targeting sequence affects the extent of transcriptional repression or activation of a target gene by CRISPRi or CRISPRa, allowing the design of sets of mismatched sgRNAs against the gene to allow its downregulation or upregulation to varying extents. In some embodiments, the information on the expression level of the target gene is encoded in the sgRNA sequence or in the vector encoding the sgRNA, and can therefore be read out by, e.g., deep sequencing and matched to a resulting phenotype. In such embodiments, experiments involving systematically mismatched sgRNAs can be conducted in a single pooled experiment, reducing experimental variation and enhancing reproducibility. It will be appreciated that any of the herein-described methods and compositions can be applied to both gene downregulation (using CRISPRi) and overexpression (using CRISPRa), as well as to other dCas9-mediated applications such as dCas9-based epigenetic modifiers.

In another aspect, the present disclosure provides specific mutations in the sgRNA constant region that lower or increase the extent of transcriptional repression or activation of a target gene by CRISPRi or CRISPRa. Using the present methods and compositions, it is therefore possible to control the expression of a number of different genes by designing multiple sgRNAs comprising different modifications in the sgRNA constant region that each give rise to a discrete level of expression of the targeted gene. Similar to the herein-disclosed methods involving mismatches in the targeting sequence of sgRNAs, methods are also provided to introduce specific mutations in the sgRNA constant region, and specific rules and parameters are provided for the design of sgRNAs comprising such mutations. In addition, a table is provided (Table 6) disclosing close to 1000 different constant region mutations and providing a ranking of their relative effects on CRISPRi or CRISPRa activity. Any one or more of these mutations can be used to modulate the expression level of any gene of interest according to the present methods.

The two different types of sgRNA modifications provided herein, i.e., comprising mismatches in the sgRNA targeting sequence and comprising mutations in the sgRNA constant region, can be combined in any way. For example, a single sgRNA can comprise both types of modification, and/or sets or libraries of sgRNAs can be used in which certain sgRNAs comprise targeting sequence mismatches and certain sgRNAs comprise constant region modifications.

This invention affords precise control over the expression level of any mammalian gene, and as such can be used in any of a large number of potential applications. For example, the methods and compositions can be used to profile the phenotypes resulting from varying degrees of downregulation or upregulation for every gene, providing information on the relationship between expression level and phenotype. The methods and compositions are also applicable to determining the cellular target and mechanism of action of, e.g., drugs with unknown mechanisms of action, of drug candidates, or of cytotoxic agents, such as drugs, drug candidates, or cytotoxic agents arising from high-throughput chemical screening efforts.

In such embodiments, this invention could be used immediately after the chemical screen to, e.g., identify the mechanism of action of compounds of interest to guide further development and characterization. In particular, profiling drug sensitivity at varying levels of knockdown and overexpression can identify genes for which small changes in expression levels cause hypersensitivity to a drug or compound of interest.

The present methods and compositions also allow for determination of gene-gene interactions for identification of synthetic lethal interactions. Additionally, the methods and compositions can be used to control the flux through a metabolic pathway or a signaling pathway of interest and to identify bottlenecks of such pathways. In some such embodiments, the methods and compositions could be used to guide metabolic engineering and synthetic biology approaches. In addition, the methods and compositions can be used to systematically analyze phenotypes associated with partial loss-of-function of essential genes. For example, the methods and compositions can be used to assign phenotypes at different expression levels of a gene. This ability can, e.g., facilitate the study of essential genes, which cannot be studied easily as their complete loss leads to cell death, and allow for the study of partial loss-of-function phenotypes.

More generally, the present methods and compositions can be used to control the activity of any CRISPR system that relies on sgRNA-DNA base pairing. The methods and compositions can also be used to comprehensively define the propensity for off-target effects during CRISPR-mediated gene editing and develop gene editing products that are tuned to minimize off-target effects.

The present methods and compositions improve on existing technology with the ability to control activity of CRISPR systems with high precision. In particular, they modulate their activity using systematic mismatches in the sgRNA or using engineered constant region variants, which obviates the need to engineer Cas9 variants with different activities or stabilities. Applications enabled by this invention can be carried out in a single genetic background and in a single experimental vessel, thereby improving data quality. The present methods and compositions also improve on previously developed technology for drug target identification, by enabling the identification of targets with higher precision.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of an sgRNA, dCas9, or target gene and/or a nucleic acid sequence encoding a protein (e.g., a protein encoded by a target gene). In some embodiments, the term refers to the production of a transcriptional and/or translational product encoded by a gene or a portion thereof. The level of expression of a DNA molecule in a cell may be assessed, e.g., on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V, and VI. See, e.g., Fonfara et al., Nature 532, 7600 (2016); Zetsche et al., Cell 163, 759-771 (2015); Adli (2018) Nat Commun. 2018 May 15; 9(1):1911. Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

The Cas9 used in the present methods can be from any source, so long that it is capable of binding to an sgRNA of the invention and being guided to the specific DNA sequence targeted by the targeting sequence of the sgRNA. In some embodiments, Cas9 is from Streptococcus pyogenes. The Cas9 can be catalytically active, but in particular embodiments the Cas9 used in the present methods is nuclease deficient, i.e., dCas9, used either alone or as a fusion protein with another functional element such as a transcriptional modulator. In particular embodiments, the Cas9 is a dCas9 protein fused to a transcriptional repressor such as KRAB (i.e., for use in CRISPRi-based methods) or is a dCas9 protein fused to a transcriptional activator such as VP64 (i.e., for use in CRISPRa-based methods).

The sgRNAs, or single guide RNAs, used herein can be any sgRNA that can function with an endogenous or exogenous CRISPR-Cas9 system, e.g., to direct the induction of deletions or gene repression in cells, or more generally to bind to the Cas9 protein and direct it to a specific target DNA sequence determined by the targeting sequence in the sgRNA. Specifically, an sgRNA interacts with a site-directed nuclease such as Cas9 or dCas9 and specifically binds to or hybridizes to a target nucleic acid within the genome of a cell, such that the sgRNA and the site-directed nuclease co-localize to the target nucleic acid in the genome of the cell. Typically, the sgRNAs as used herein comprise a targeting sequence comprising homology (or complementarity) to a target DNA sequence in the genome, and a constant region that mediates binding to Cas9 or another site-directed nuclease. In particular embodiments, the targeting sequence may comprise one or more mismatches with the target DNA sequence, and/or the constant region may contain one or more mutations, as described in more detail elsewhere herein.

### 3. Detailed Description of the Embodiments

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Provided herein are compositions and methods for generating discrete, intermediate expression levels of any gene of interest when using CRISPRi or CRISPRa. In particular, the present compositions and methods involve the introduction of one or more mismatches or mutations into the targeting sequence or constant region of sgRNAs so as to achieve a level of CRISPRi or CRISPRa activity that is, e.g., intermediate between that obtained with an sgRNA sharing 100% homology with a target DNA sequence and/or an unmodified constant region and that obtained with a scrambled sgRNA and/or sgRNA comprising a modified constant region providing no CRISPRi or CRISPRa activity on the gene in question. Further, rules are provided by which the specific effects of a given mismatch or mutation on CRISPRi or CRISPRa activity can be determined, allowing the design of sets of sgRNAs targeting a given gene and providing a series of discrete levels of expression of the gene. As described herein, such sets can be combined to form libraries targeting multiple genes, including large libraries targeting thousands of genes in the genome.

sgRNAs

The sgRNAs of the invention comprise two or more regions, including a “targeting sequence” that is complementary to, and thus targets, a target DNA sequence in the template DNA, e.g., the promoter region or region surrounding the transcription start site, and thereby modulate its expression using CRISPRi or CRISPRa. The sgRNAs also comprise a “constant region” that mediates its interaction with an sgRNA-guided nuclease such as Cas9 (e.g., dCas9).

The sgRNAs used in the present methods can also comprise additional functional or structural elements, such as barcodes to provide a specific distinct sequence for each sgRNA in a set or a library, restriction sites, primer sites, and the like.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgDNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence). In particular embodiments, the region of the sgRNA that is considered with respect to homology or complementarity for the purposes of the present methods is the last 19 nucleotides in the sgRNA that lead up to the PAM sequence in the target DNA. Accordingly, in some embodiments these 19 nucleotides are 100% homologous or complementary to the template DNA, and in some embodiments this 19-nucleotide region includes one or more mismatches with the target DNA sequence. In some embodiments, the region of the sgRNA that is considered with respect to homology or complementarity for the purposes of the present methods is the region from the second nucleotide of the sgRNA up to the PAM sequence in the target DNA, regardless of the length of the region. Accordingly, in some embodiments the sequence starting at the second nucleotide of the sgRNA and leading up to the PAM is 100% complementary to the target DNA sequence. In some embodiments the sequence starting at the second nucleotide of the sgRNA and leading up to the PAM comprises one or more mismatches with the target DNA sequence.

In some cases, G-C content of the sgRNA is preferably between about 40% and about 60% (e.g., 40%, 45%, 50%, 55%, 60%). In some cases, the targeting sequence can be selected to begin with a sequence that facilitates efficient transcription of the sgRNA. For example, the targeting sequence can begin at the 5′ end with a G nucleotide. In some cases, the binding region or the overall sgRNA can contain modified nucleotides such as, without limitation, methylated or phosphorylated nucleotides. In some embodiments, the sgRNAs selected for use in the present methods are filtered by identifying and eliminating potential targeting sequences that are likely to or could potentially give rise to significant off-target effects (i.e., if the targeting sequence is substantially homologous or complementary to one or more sequences within the genome other than the target DNA sequence). In some embodiments, sgRNAs comprising internal restriction sites recognized by restriction enzymes that may be used in one or more cloning steps of the methods may be excluded as well.

As used herein, the term “complementary” or “complementarity” refers to base pairing between nucleotides or nucleic acids, for example, and not to be limiting, base pairing between a sgRNA and a target nucleic acid. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence.

In some embodiments, the sgRNAs are targeted to specific regions at or near a gene, e.g., to a region at or near the 0-1000 bp region 5′ (upstream) of the transcription start site of a gene, or to a region at or near the 0-1000 bp region 3′ (downstream) of the transcription start site of a gene.

In some embodiments, the sgRNAs are targeted to a region at or near the transcription start site (TSS) based on an automated or manually annotated database. For example, transcripts annotated by Ensembl/GENCODE or the APPRIS pipeline (Rodriguez et al., Nucleic Acids Res. 2013 January; 41(Database issue):D110-7 can be used to identify the TSS and target genetic elements 0-750 bp or 0-1000 bp downstream of the TSS.

In some embodiments, the sgRNAs are targeted to a genomic region that is predicted to be relatively free of nucleosomes. The locations and occupancies of nucleosomes can be assayed, e.g., through the use of enzymatic digestion with micrococcal nuclease (MNase). MNase is an endo-exo nuclease that preferentially digests naked DNA and the DNA in linkers between nucleosomes, thus enriching for nucleosome-associated DNA. To determine nucleosome organization genome-wide, DNA remaining from MNase digestion is sequenced using high-throughput sequencing technologies (MNase-seq). Thus, regions having a high MNase-seq signal are predicted to be relatively occupied by nucleosomes, and regions having a low MNase-seq signal are predicted to be relatively unoccupied by nucleosomes. Thus, in some examples, the sgRNAs are targeted to a genomic region that has a low MNase-Seq signal.

In some embodiments, the sgRNAs are targeted to a region predicted to be highly transcriptionally active. For example, the sgRNAs can be targeted to a region predicted to have a relatively high occupancy for RNA polymerase II (PolII). Such regions can be identified by PolII chromatin immunoprecipitation sequencing (ChIP-seq), which includes affinity purifying regions of DNA bound to PolII using an anti-PolII antibody and identifying the purified regions by sequencing. Therefore, regions having a high PolII Chip-seq signal are predicted to be highly transcriptionally active. Thus, in some cases, sgRNAs are targeted to regions having a high PolII ChIP-seq signal as disclosed in the ENCODE-published PolII ChIP-seq database (Landt, et al., Genome Research, 2012 September; 22(9):1813-31).

In some such embodiments, the sgRNAs can be targeted to a region predicted to be highly transcriptionally active as identified by run-on sequencing or global run-on sequencing (GRO-seq). GRO-seq involves incubating cells or nuclei with a labeled nucleotide and an agent that inhibits binding of new RNA polymerase to transcription start sites (e.g., sarkosyl). Thus, only genes with an engaged RNA polymerase produce labeled transcripts. After a sufficient period of time to allow global transcription to proceed, labeled RNA is extracted and corresponding transcribed genes are identified by sequencing. Therefore, regions having a high GRO-seq signal are predicted to be highly transcriptionally active. Thus, in some cases, sgRNAs are targeted to regions having a high GRO-seq signal as disclosed in a published GRO-seq data (e.g., Core et al., Science. 2008 Dec. 19; 322(5909):1845-8; and Hah et al., Genome Res. 2013 August; 23(8):1210-23).

Each sgRNA also includes a constant region that interacts with or binds to the site-directed nuclease, e.g., Cas9 or dCas9. In the nucleic acid constructs provided herein, the constant region of an sgRNA can be from about 75 to 250 nucleotides in length, or about 75-100 nucleotides in length, or about 85-90 nucleotides in length, or 75, 76, 77, 7, 79, 80, 81, 82, 83, 84, 85, 86, 87, 88, 89, 90, 91, 92, 93, 94, 95, 96, 97, 98, 99, 100 or more nucleotides in length. In some examples, as described in more detail elsewhere herein, the constant region is modified, e.g., comprises one or more nucleotide substitutions in the first stem loop, the second stem loop, a hairpin, a region in between the hairpins, and/or the nexus of a constant region, so as to generate intermediate levels of CRISPRi or CRISPRa activity between the levels obtained using an sgRNA with a non-modified constant region and those obtained using an sgRNA with a modified constant region that provides no CRISPRi or CRISPRa activity, e.g., by virtue of being incapable of functionally interacting with Cas9. In some embodiments, mutations in the constant region can confer CRISPRi or CRISPRa activity that is greater than that obtained using an sgRNA with an unmodified constant region.

A non-limiting example of an unmodified constant region that can be used in the constructs set forth herein is shown as cr995 in Table 6. Other constant regions that can be used are described in Gilbert et al. (2014) Cell, 159(3): 647-661, the entire disclosure of which is herein incorporated by reference. In addition, a non-limiting list of modified constant regions that include one or more mutations in the constant region, is provided herein in Table 6. Any of the constant regions or mutations shown in Table 6 can be used in the present methods.

### Mismatches in the Targeting Sequence

In some embodiments, sgRNAs are provided with one or more mismatches in the targeting sequence of the sgRNA in order to generate intermediate levels of CRISPRi or CRISPRa activity. In particular embodiments, the mismatches introduced into the targeting sequence are in the last 19 nucleotides of the targeting region, i.e., the 19 nucleotides leading up to the PAM sequence in the target DNA. In some embodiments, the mismatches introduced into the targeting sequence are in the region from the second nucleotide of the sgRNA leading up to the PAM sequence in the target DNA. In some embodiments, sets of sgRNAs are provided with different mismatches so as to obtain a series of different expression levels of a target gene. A set typically includes at least one sgRNA in which this 19 nucleotide region, or in which the region from the second nucleotide of the sgRNA to the PAM, is 100% homologous to the template DNA, as well as one or more sgRNAs that comprise one or more mismatches within the 19 nucleotide region or within the region from the second nucleotide to the PAM. Mismatches in the targeting sequence selected according to the present methods reduce the CRISPRi or CRISPRa activity to an intermediate level between that of an sgRNA with 100% homology to the target DNA (e.g., providing 100% CRISPRi or CRISPRa activity) and that of a scrambled sgRNA that does not target the target DNA (i.e., with a targeting sequence comprising insufficient homology to the target DNA sequence to promote Cas9 binding and consequent CRISPRi or CRISPRa activity). It will be appreciated that a given gene can be targeted using a single set of sgRNAs that recognize a single target sequence within the gene, or with multiple sets that each target a different DNA sequence within the target gene.

In some embodiments, an sgRNA comprising one or more mismatches in the targeting sequence provides about 5%, 10%, 15%, 20%, 25%, 30%, 35%, 40%, 45%, 50%, 55%, 60%, 65%, 70%, 80%, 85%, 90%, or 95% CRISPRi or CRISPRa activity, wherein 100% CRISPRi or CRISPRa activity corresponds to the activity in the presence of an sgRNA targeting the same DNA sequence and comprising 100% homology to the target sequence, and wherein 0% CRISPRi or CRISPRa activity corresponds to the activity in the presence of a scrambled sgRNA with no, or only insignificant amounts of, homology to the target sequence.

Any of a number of parameters can be used to select a mismatch in the targeting sequence of the sgRNA, i.e., in the last 19 nucleotides of the targeting sequence leading up to the PAM, or in the region from the second nucleotide of the sgRNA leading up to the PAM, in order to obtain a predictable, intermediate level of CRISPRi or CRISPRa activity. For example, in some embodiments, the mismatch is selected on the basis of its distance from the PAM sequence. More precisely, the mismatch is selected on the basis of the following positional relationships, with the position indicated (e.g., −19) counted as the number of bases upstream from the sgRNA PAM, and the positions ordered by how much CRISPRi or CRISPRa activity the sgRNAs provide:

−19>−18>−17>−16≈−15≈−14>−13>−12>−11>−10>−9>−8>−4>−7≈−6≈−5≈−3≈−2≈−1

For example, an sgRNA with a mismatch in position −19 will on average have higher activity (that is, mediate stronger knockdown/overexpression by CRISPRi or CRISPRa, respectively) than an sgRNA with a mismatch in position −11.

Another parameter that can be used to select a mismatch in the targeted sequence is the identity of the nucleotides involved in pairing in the mismatched position:

rG:dT>rU:dG>rG:dA≈rG:dG>rC:dA>rU:dT>rA:dA>rC:dT>rA:dC>rA:dG>rU:dC≈rC:dC
with the identity of the mismatch indicated as rX:dY for “base X in the sgRNA opposite base Y in the DNA” (i.e., the 4 non-mismatched pairs would be rG:dC, rC:dG, rA:dT, rU:dA). As with the relative activities determined by the position of the mismatch relative to the PAM, the pairings indicated here are ordered by how much CRISPRi or CRISPRa activity the sgRNAs on average retain relative to an sgRNA with 100% homology to the target DNA (e.g., a mismatched sgRNA with a rG:dT pairing will have higher CRISPRi or CRISPRa activity than a mismatched sgRNA with a rC:dT pairing, all else being equal).

In some embodiments, the mismatches introduced into sgRNA targeting sequences are selected by taking into account both the position and the identity of the nucleotides involved in the basepairing, in particular according to the following ranking that groups together different mismatch positions:

If the mismatch is between position −19 and −13 (both inclusive):

rG:dT>rC:dA>rU:dG≈rG:dA≈rU:dT≈rC:dT≈rA:dA>rG:dG>rA:dC≈rA:dG>rU:dC≈rC:dC

If the mismatch between position −12 and −9 (both inclusive):

rG:dT>rU:dG≈rG:dA≈rG:dG>rU:dT>rC:dA≈rC:dT>rA:dA>rA:dC≈rA:dG>rU:dC≈rC:dC

If the mismatch between position −8 and −1 (both inclusive):

rG:dT>rG:dA≈rC:dA>rU:dG≈rG:dG>rA:dA≈rU:dT≈rA:dC>rC:dT>rA:dG≈rU:dC≈rC:dC

In some such embodiments, a set of sgRNAs is designed and/or prepared in which at least one sgRNA has a mismatch between positions −19 and −13, at least one has a mismatch between positions −12 and −9, and at least one has a mismatch between positions −8 and −1.

In some embodiments, the mismatches introduced into the sgRNA targeting sequences are selected by taking into account the identity of the nucleotides immediately surrounding the mismatch. For example, the activity of mismatched sgRNAs is generally higher if there is a G (in the sgRNA sequence) either immediately upstream or 1, 2, or 3 nucleotides downstream of the mismatch, and particularly so if there is a G both before and after the mismatch. Further, the activity of mismatched sgRNAs is generally lower if lower if there is a U either immediately upstream or 1, 2, or 3 nucleotides downstream of the mismatch, and particularly so if there is a U both before and after the mismatch.

In some embodiments, the mismatches introduced into the sgRNA targeting sequences are selected based on the general rule that the higher the GC content that a mismatched sgRNA has, the greater is its CRISPRi or CRISPRa activity.

Any of these rules and parameters can be used alone or in any possible combination to prepare an sgRNA with a desired level of CRISPRi or CRISPRa activity, and to prepare sets of sgRNAs targeting a single gene (i.e., a single set targeting a single DNA sequence within the gene, or multiple sets each targeting a different DNA sequence within the gene), wherein the set or sets comprise multiple sgRNAs that give rise to a series of different levels of expression of the targeted gene (e.g. with reduced expression levels using CRISPRi or increased expression levels using CRISPRa).

It will be appreciated that the specific expression of the target gene using a given sgRNA will depend to some extent upon, e.g., the gene that is being targeted, the specific DNA sequence within the target gene that is being targeted, the nature of the mismatches in the targeting sequence vis-a-vis the target DNA, and whether the sgRNA is used with CRISPRi or CRISPRa. Using the herein-described methods, however, it is possible to generate a set of sgRNAs that predictably cover any desired range of expression levels of a gene using CRISPRi or CRISPRa, e.g., cover any range of expression levels between 1% and 5000% of the normal expression level of the gene.

### Assessment of Off-Target Effects

Introducing mismatches into the sgRNA targeting sequence may increase the potential for binding at non-intended genomic sites, or off-target effects. Such off-target potential can be assessed using two different strategies. In a first strategy, a FASTQ entry is created for the 23 bases of each genomic target in the genome including the PAM, with the accompanying empirical Phred score indicating an estimate of the anticipated importance of a mismatch in that base position. By aligning each sgRNA sequence back to the genome, parameterized so that sgRNAs are considered to mutually align if and only if: a) no more than 3 mismatches existed in the PAM-proximal 12 bases and the PAM, b) the summed Phred score of all mismatched positions across the 23 bases was less than a threshold, for example using Bowtie or similar software, it can be determined if a given sgRNA has no other binding sites in the genome at a given threshold. By performing this alignment iteratively with decreasing thresholds, an off-target specificity can be assigned to each sgRNA.

In a second strategy, empirical measurements of activities of sgRNAs comprising mismatches can be used to calculate the off-target potential. In a first step, all potential off-target sites up to 3 mismatches away for each sgRNA are determined, for example using Cas-OFFinder or a related method. These off-target sites can then be aggregated into a specificity score for each sgRNA:

$$specificity score = \frac{1}{{Σ}_{i = 1}^{n} R {A}_{i} \cdot {q}_{i}}$$

Where n represents the number of sites with up to 3 mismatches, RA the empirically measured relative CRISPRi activity of each sgRNA at this target site given the positions and types of mismatches, and q the number of times the ith site occurs in the genome. In particular, RA can be calculated as follows:

RA=Πⱼ₌₁^{m}RAⱼ

Where m represents the number of mismatches between the sgRNA and the target site and RAⱼ represents the mean relative activity of sgRNAs with mismatch j (given mismatch type at given sgRNA position). Because many sgRNAs by definition contain mismatches to the intended on-target site, the RA of the intended on-target site is assigned a value of 1 to keep the specificity scores on a scale of 0 to 1. A specificity score of 1 indicates that there are no off-target sites with up to 3 mismatches in the genome, whereas a specificity score of 0.001 indicates nearly complete lack of specificity.

By appropriately calculating off-target potential for sgRNAs comprising mismatches, off-target effects can be minimized.

### Modifications in the Constant Sequence

In some embodiments, sgRNAs are provided with one or more nucleotide changes into the sgRNA constant region (i.e., in the region outside of the targeting sequence that is required for binding to Cas9) so as to obtain intermediate levels of CRISPRi or CRISPRa activity, or in some cases levels that exceed those obtained with an unmodified constant region. In some embodiments, sets of sgRNAs are provided comprising individual sgRNAs with different mutations so as to obtain a series of different expression levels of a target gene. In such embodiments, an sgRNA will typically be used in which the constant region is not modified, e.g., is 100% identical to the sequence shown as constant region cr995 in Table 6, and one or more additional sgRNAs will also be used that comprise one or more nucleotide or base-pair substitutions within the constant region.

A list of sgRNAs comprising 995 constant region variants, comprising all possible single nucleotide substitutions, base pair substitutions, and combinations of these changes is provided herein and shown in Table 6, along with their ranking and with the mean CRISPRi or CRISPRa activities that they confer. Any of these modified sgRNA sequences can be used in the present methods. In particular embodiments, a set of sgRNAs generating a series of discrete expression levels by CRISPRi or CRISPRa is produced using a plurality of such variants, e.g., by selecting a plurality of variants according to their ranking in Table 6. As indicated in Table 6, in some embodiments a constant region mutation will generate CRISPRi or CRISPRa activity levels that are greater than those obtained with an unmodified constant region. As such, using such modifications it is possible to generate sets of sgRNAs that cover expression levels that are both intermediate between those obtained with an unmodified constant region and those obtained with a modified region that provides no CRISPRi or CRISPRa activity, as well as expression levels that exceed those obtained with an unmodified constant region.

In some embodiments, sgRNA variants with modifications in their constant regions are selected based on one or more rules or parameters, e.g., rules or parameters deduced from the rankings shown in Table 6. For example, the mutation of bases known to mediate contacts with Cas9 (e.g., in the first stem-loop or the nexus) gives rise to greater CRISPRi or CRISPRa activity than mutations in regions not contacted by Cas9 (e.g., in the hairpin region of stem-loop 2). In some embodiments, sets are provided by selecting a plurality of sequences or mutations listed in Table 6 according to the ranking provided and/or the mean relative activities indicated, so as to obtain a plurality of gene expression levels by CRISPRi or CRISPRa.

It will be appreciated that the specific expression of the target gene using a given sgRNA will depend to some extent upon, e.g., the gene that is being targeted, the specific DNA sequence within the target gene that is being targeted, the nature of the mutation in the constant region, and whether the sgRNA is used with CRISPRi or CRISPRa. Using the herein-described methods, however, it is possible to generate a set of sgRNAs that predictably cover any desired range of expression levels of a gene using CRISPRi or CRISPRa, e.g., cover any range of expression levels between 1% and 5000% of the normal expression level of the gene.

sgRNA Sets and Libraries

In particular embodiments, the present disclosure provides sets and libraries of sgRNAs generated using the herein-described methods, i.e., introducing mismatches into the sgRNA targeting sequence and/or introducing modifications into the sgRNA constant region. For example, a set of sgRNAs can be designed and prepared to target a single gene and, when introduced into a plurality of cells, generate a series of discrete expression levels of the gene by CRISPRi or CRISPRa. The sets of sgRNAs will typically include a “wild-type” sgRNA, i.e., an sgRNA with 100% homology to the target DNA sequence in the 19 nucleotides leading up to the PAM and/or an sgRNA with no modifications in the constant region, as well as one or more additional sgRNAs with one or more mismatches in the targeting sequence and/or modifications in the constant region. The sets also optionally include a negative control sgRNA providing no CRISPRi or CRISPRa activity, e.g., an sgRNA with a scrambled targeting sequence or with sufficient modifications in the constant region to abolish Cas9 binding and therefore CRISPRi or CRISPRa activity.

Accordingly, in some embodiments, a set of sgRNAs is provided comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more structurally distinct sgRNAs targeting a single gene, or targeting a single target sequence within a single gene. In some embodiments, the different sgRNAs of the set provide a series of discrete expression levels of the targeted gene. For example, an individual mismatched or modified sgRNA in the set may provide about 5%, 10%, 15%, 20%, 25%, 30%, 35%, 40%, 45%, 50%, 55%, 60%, 65%, 70%, 75%, 80%, 85%, 90%, 95%, 105%, or 110% CRISPRi or CRISPRa activity, or any percentage value from 1% to 110%, as compared to a non-mismatched or unmodified sgRNA. In some embodiments, a set is generated in which at least one sgRNA is provided that generates a level of CRISPRi or CRISPRa activity within each of multiple windows of activity. For example, a set can contain one or more sgRNAs that provide from about 1%-50% activity and one or more sgRNAs that provide from about 51%-99% activity; or a set can comprise one or more sgRNAs that provide about 1%-33% activity, one or more sgRNAs that provide about 34%-66% activity, and one or more sgRNAs that provide about 67-99% activity; or a set can comprise one or more sgRNAs that provide about 1%-25% activity, one or more sgRNAs that provide about 26%-50% activity, one or more sgRNAs that provide about 51%-75% activity, and one or more sgRNAs that provide about 76%-99% activity; or a set can comprise one or more sgRNAs that provide about 1%-10% activity, one or more sgRNAs that provide about 11%-20% activity, one or more sgRNAs that provide about 21-30% activity, one or more sgRNAs that provide about 31%-40% activity, one or more sgRNAs that provide about 41-50% activity, one or more sgRNAs that provide about 51%-60% activity, one or more sgRNAs that provide about 61-70% activity, one or more sgRNAs that provide about 71%-80% activity, one or more sgRNAs that provide about 81-90% activity, and one or more sgRNAs that provide about 91%-99% activity. In some embodiments, one or more sgRNAs provide about 10%-30% activity, one or more sgRNAs provide about 30-50% activity, one or more sgRNAs provide about 50%-70% activity, and one or more sgRNAs provide about 70-90% activity.

In some embodiments, in particular with certain constant region mutations, a set will further include one or more sgRNAs that provide greater than 100% activity, e.g., 101%, 102%, 103%, 104%, 105%, 106%, 107%, 108%, 109%, 110%, or higher.

In some embodiments, the present disclosure provides libraries of sgRNAs comprising multiple sets of sgRNAs, with each set of sgRNAs targeting an individual gene or a specific target DNA within a gene. Accordingly, in some embodiments, a library of sgRNAs is provided comprising about 1000, 5000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000 or more structurally distinct sgRNAs, or a library of sgRNAs is provided comprising 2 or more sets of sgRNAs targeting about 100, 200, 300, 400, 500, 600, 700, 800, 900, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000 or more individual gene targets. In some embodiments, the library of sgRNAs targets a group of genes involved in a common pathway, process, or biological or physiological activity, or targets a group of genes known to produce a common phenotype. In some embodiments, all of the genes in the genome, or substantially all of the genes in the genome, are targeted.

For preparing a set of sgRNAs or a library of sgRNAs, once the target gene and the target DNA sequence or sequences within the genes have been selected, and the desired range of expression levels has been determined, a plurality of sgRNAs is designed using the herein-described rules, factors, parameters, and rankings of Table 6 for selecting mismatches in the sgRNA targeting sequence and/or mutations within the sgRNA constant region so as to obtain a set of sgRNAs that provide the desired expression levels of the targeted genes using CRISPRi or CRISPRa. In some embodiments, e.g., for sets comprising sgRNAs with mismatches in the targeting sequence, a set will comprise sgRNAs that each have mismatches in each of different regions of the targeting sequence. For example, in some embodiments, a set contains one or more sgRNAs with mismatches within 7 nucleotides of the PAM, the set contains one or more sgRNAs with mismatches located 8-12 nucleotides upstream of the PAM, and the set contains one or more sgRNAs with mismatches located 13-19 nucleotides upstream of the PAM.

In some embodiments, additional steps are included to exclude certain potential sgRNAs from a set or library. For example, a step can be included in which mismatched sgRNAs are assessed for potential off-target binding, and sgRNAs that are predicted to have or that have a potential for significant off-target binding are not used. In such embodiments, for example, for a given target in the genome, a FASTQ entry is created for the 23 bases of the target including the PAM, and the accompanying empirical Phred score is used to indicate an estimate of the anticipated importance of a mismatch at each position. Bowtie (bowtie-bio.sourceforge.net), e.g., is then used to align each designed sgRNA back to the genome, parameterized so that sgRNAs are considered to mutually align if and only if: a) no more than 3 mismatches exist in the PAM-proximal 12 bases and the PAM, and b) the summed Phred score of all mismatched positions across the 23 bases is below a threshold value. This alignment can be done iteratively with decreasing thresholds, and any sgRNAs that align successfully to no other site in the genome at a given threshold are deemed to have specificity at the threshold.

Other steps to filter potential sgRNAs can also be included, for example to exclude sgRNAs comprising one or more restriction sites that may be used for subsequent cloning or sequencing library preparation, such as BstXI, BlpI, and/or SbfI.

### Applications

This invention affords precise control over the expression level of any mammalian gene, and as such can be used in any of a large number of potential applications. For example, in some embodiments a method is provided to profile the phenotypes resulting from varying degrees of downregulation or upregulation for every gene, providing information on the relationship between expression level and phenotype. Further, in some embodiments a method is provided to determine the cellular target and mechanism of action of, e.g., drugs with unknown mechanisms of action, of drug candidates, or of cytotoxic agents, such as drugs, drug candidates, or cytotoxic agents arising from high-throughput chemical screening efforts. In such embodiments, the present methods can be used immediately after the chemical screen to, e.g., identify the mechanism of action of compounds of interest to guide further development and characterization. In particular, the methods can be used to profile drug sensitivity at varying levels of knockdown and overexpression in order to identify genes for which small changes in expression levels cause hypersensitivity to a drug or compound of interest.

In some embodiments, a method is provided to determine gene-gene interactions for identification of synthetic lethal interactions. Additionally, a method is provided to control the flux through a metabolic pathway or a signaling pathway of interest and to identify bottlenecks of such pathways. In some such embodiments, the methods and compositions are used to guide metabolic engineering and synthetic biology approaches. In some embodiments, a method is provided to systematically analyze phenotypes associated with partial loss-of-function of essential genes. In some embodiments, a method is provided to assign phenotypes at different expression levels of a gene. In some such embodiments, the method is used to study an essential gene, which cannot be studied easily as its complete loss would lead to cell death, and to study partial loss-of-function phenotypes of the gene.

Also provided are methods to control the activity of any CRISPR system that relies on sgRNA-DNA base pairing. For example, the methods can also be used to comprehensively define the propensity for off-target effects during CRISPR-mediated gene editing and develop gene editing products that are tuned to minimize off-target effects.

In some embodiments, methods are provided to identify the functionally sufficient levels of gene products, which can serve as targets for rescue by gene therapy or chemical treatment when genes are affected by disease-causing loss-of-function mutations or as targets of inhibition for anti-cancer drugs, such that proliferating cancer cells experience toxicity while healthy cells are spared. In some embodiments, methods are provided to titrate the expression of individual genes in mammalian systems.

In some embodiments, a method is provided to identify the therapeutic window for restoration of a gene, e.g., a disease-associated gene whose loss-of-function leads to a disease-associated phenotype. In some such embodiments, a cell model is used that has normal levels of the disease-associated gene, but where deletion of the gene (or otherwise eliminating gene function) results in a measurable, e.g., disease-relevant, phenotype. In some such embodiments, the present methods are used with, e.g., CRISPRi to titrate the gene, i.e., produce multiple, decreased expression levels of the gene, and define the expression level at which the disease phenotype is alleviated to a relevant extent. In other such embodiments, a cell model is used that has a loss-of-function mutation in the disease-associated gene and a measurable phenotype, and the disease-associated gene is reintroduced, the resulting absence of the phenotype verified, and the expression of the reintroduced gene titrated using the present methods to define the expression level of the gene at which the disease phenotype is alleviated. It will be appreciated that such methods can be used to define the particular expression level required to alleviate or alter any measurable phenotype in any cell type, not only those associated with a disease.

In other embodiments, a method is provided of determining a therapeutic window for the inhibition of a gene, for example to lower the expression of a gene for therapeutic purposes but where elimination of the expression of the gene would be lethal or otherwise deleterious. Such methods can be used, e.g., to identify the lowest possible level of the gene that provides a therapeutic benefit but which is still compatible with survival or with otherwise avoiding the deleterious effects associated with complete loss of the gene. In some such embodiments, the relationship between decreased expression levels of the gene and the survival or growth of the cells is determined according to the herein-described methods for a plurality of sgRNAs targeting the gene using CRISPRi, and wherein the minimum level of expression of the gene that is compatible with cell growth or survival is determined, thereby determining the lower boundary of the therapeutic window for the inhibition of the gene.

In other embodiments, methods are provided of identifying a gene target of a cytotoxic agent or a drug candidate. In some such methods, a population of test cells is generated according to the present methods, where each test cell within the population expresses dCas9, e.g., dCas9 fused to a transcriptional repressor, as well as one or more sgRNAs of the invention, and the population of test cells is contacted with a sub-lethal or sub-therapeutic amount of the cytotoxic agent or drug candidate. The test cells within the population are then examined to identified test cells that display a phenotype in the presence of the sub-lethal or sub-therapeutic amount of the cytotoxic agent or drug candidate that is not displayed by cells in the absence of the dCas9 or of an sgRNA, and then the identity of the sgRNAs, and of the genes targeted by the sgRNAs, present within those phenotype-displaying test cells is determined. Genes that are targeted by one or more distinct sgRNAs in cells displaying a phenotype are candidate gene targets of the cytotoxic agent or drug candidate.

Preparation of sgRNAs, sgRNA Sets and Libraries

The sgRNAs provided herein can be synthesized using standard methods. For example, two complementary oligonucleotides (e.g., as synthesized using standard methods or obtained from a commercial supplier, e.g., Integrated DNA Technologies) containing the targeting region as well as overhangs matching those left by restriction digestion (e.g., by BstXI and/or BlpI) of an appropriate expression vector, can be annealed and ligated into an sgRNA expression vector digested using the same restriction enzymes. The ligated product is then transformed into competent cells (e.g., E. coli, e.g. as obtained from Takara Bio) and the plasmid prepared using standard protocols. Methods of synthesizing and preparing sgRNAs of the invention are disclosed, e.g., in Gilbert et al. Cell (2014) 159:647-661, the disclosure of which is herein incorporated in its entirety by reference.

In some embodiments, sgRNAs are ligated into sgRNA expression vectors such as pU6 vectors (i.e., vectors comprising CRISPR-Cas9 elements), e.g., a pU6-sgCXCR4-2 vector which also comprises a puromycin resistance cassette and mCherry. Such vectors can be obtained, e.g., from commercial suppliers (e.g., Addgene). sgRNA vectors can then be introduced into mammalian cells, e.g., by packaging the vectors in, e.g., lentivirus and transduced using standard methods into cells, e.g., K562 or Jurkat cells, which can then be grown and analyzed (e.g., by FACS, to record and/or gate on the basis of, e.g., GFP or mCherry expression).

Pooled sgRNA libraries can be cloned, e.g., as described in Gilbert et al., Cell (2014) 159:647-661; Kampmann et al., (2013) PNAS 110:E2317-E2326; Bassik et al. (2009) Nat. Methods 6:443-445, the disclosures of which are herein incorporated by reference in their entireties, or, e.g., by obtaining oligonucleotide pools containing the desired elements and, e.g., flanking restriction sites and PCR adaptors (e.g., from Agilent Technologies). The oligonucleotide pools are then amplified by PCR, digested with appropriate restriction enzymes, and ligated into vectors such as pCRISPRia-v2 that have been digested with the same enzymes. The ligation product is then purified and transformed into competent cells, e.g., electrocompetent cells such as MegaX DH10B cells (Thermo Fisher Scientific) by, e.g., electroporation using a system such as Gene Pulser Xcell system (Bio-Rad). Following growth and appropriate selection of the cells, the pooled sgRNA plasmid library is extracted, e.g., by GigaPrep (Qiagen or Zymo Research).

### Site-Directed Nucleases

The present methods involve the expression of sgRNAs in cells along with a site-directed nuclease such as Cas9, e.g., dCas9, e.g., dCas9 fused to a transcriptional modulator. See, for example, Abudayyeh et al., Science 2016 Aug. 5; 353(6299): aaf5573; and Fonfara et al. Nature 532: 517-521 (2016). As used throughout, the term “Cas9 polypeptide” means a Cas9 protein or a fragment thereof present in any bacterial species that encodes a Type II CRISPR/Cas9 system. See, for example, Makarova et al. Nature Reviews, Microbiology, 9: 467-477 (2011), including supplemental information, hereby incorporated by reference in its entirety. For example, the Cas9 protein or a fragment thereof can be from Streptococcus pyogenes. Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 from Streptococcus pyogenes is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell. In some embodiments, a Cas9 nuclease that requires an NGG protospacer adjacent motif (PAM) immediately 3′ of the region targeted by the guide RNA I sused. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to those described in Esvelt et al., Nature Methods 10: 1116-1121 (2013).

In particular embodiments, the site-directed nuclease is a deactivated site-directed nuclease, for example, a dCas9 polypeptide. As used throughout, a dCas9 polypeptide is a deactivated or nuclease-dead Cas9 (dCas9) that has been modified to inactivate Cas9 nuclease activity. Modifications include, but are not limited to, altering one or more amino acids to inactivate the nuclease activity or the nuclease domain. For example, and not to be limiting, D10A and H840A mutations can be made in Cas9 from Streptococcus pyogenes to inactivate Cas9 nuclease activity. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity.

In some examples, the dCas9 protein is a full-length Cas9 sequence from S. pyogenes lacking the polypeptide sequence of the RuvC nuclease domain and/or the HNH nuclease domain and retaining the DNA binding function. In other examples, the dCas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retain DNA binding function.

In some examples, the deactivated site-directed nuclease, for example, a deactivated Cas9, is linked to an effector protein. Optionally, the site-directed nuclease is linked to the effector protein via a peptide linker. The linker can be between about 2 and about 25 amino acids in length. The effector protein can be a transcriptional regulatory protein or an active fragment thereof. The transcriptional regulatory protein can be a transcriptional activator or a transcriptional repressor protein or a protein domain of the activator protein or the inhibitor protein. Examples of transcriptional activators include, but are not limited to VP16, VP48, VP64, VP192, MyoD, E2A, CREB, KMT2A, NF-KB (p65AD), NFAT, TET1, p300Core and p53. Examples of transcriptional inhibitors include, but are not limited to KRAB, MXI1, SID4X, LSD1, and DNMT3A/B. The effector protein can also be an epigenome editor, such as, for example, histone acetyltransferase, histone demethylase, DNA methylase etc.

The effector protein or an active fragment thereof can be operatively linked, in series, to the amino-terminus or the carboxy-terminus of the site-directed nuclease, for example, to dCas9. Optionally, two or more activating effector proteins or active domains thereof can be operatively linked to the amino-terminus or the carboxy-terminus of dCas9. Optionally, two or more repressor effector proteins or active domains thereof can be operatively linked, in series, to the amino-terminus or the carboxy-terminus of dCas9. Optionally, the effector protein can be associated, joined or otherwise connected with the nuclease, without necessarily being covalently linked to dCas9.

### Polynucleotides and Cells

In some embodiments, the compositions of the invention are introduced into cells using nucleic acid constructs. Nucleic acid constructs of the invention, e.g., polynucleotides encoding expression cassettes encoding sgRNAs or encoding dCas9, can be in any of a number of forms, e.g., in a vector, such as a plasmid, a viral vector, a lentiviral vector, etc. In some examples, the nucleic acid construct is in a host cell. The nucleic acid construct can be episomal or integrated in the host cell. The compositions provided herein can be used to modulate expression of target nucleic acid sequences in eukaryotic cells, animal cells, plant cells, fungal cells, and the like. Optionally, the cell is a mammalian cell, for example, a human cell. The cell can be in vitro or ex vivo. The cell can also be a primary cell, a germ cell, a stem cell or a precursor cell. The precursor cell can be, for example, a pluripotent stem cell or a hematopoietic stem cell. Introduction of the composition into cells can be cell cycle dependent or cell cycle independent. Methods of synchronizing cells to increase a proportion of cells in a particular phase are known in the art. Depending on the type of cell to be modified, one of skill in the art can readily determine if cell cycle synchronization is necessary.

The compositions described herein can be introduced into the cell via microinjection, lipofection, nucleofection, electroporation, nanoparticle bombardment, and the like. The compositions can also be packaged into viral particles for infection into cells.

Also provided are cells including the compositions described herein and cells modified by the compositions described herein. Cells or populations of cells comprising one or more nucleic acid constructs described herein are also provided. For example, a cell is provided comprising a nucleic acid construct comprising an expression cassette encoding an sgRNA of the invention, operably linked to a promoter, and/or a nucleic acid construct comprising an expression cassette encoding dCas9, operably linked to a promoter. Populations of cells are also provided, for example with each cell among the population comprising an expression cassette encoding a dCas9 protein, operably linked to a promoter, and comprising an expression cassette encoding one of the sgRNAs of the invention, operably linked to a promoter. In some embodiments, the sgRNA comprises a mismatch in the targeting sequence. In some embodiments, the sgRNA comprises a mutation in the constant region. In some embodiments, the sgRNA is present within a nucleic acid construct that also comprises an expression cassette encoding a unique guide barcode, e.g., as described in Adamson et al. (2016) Cell 167:1867-1882.e21, the entire disclosure of which is herein incorporated by reference). In some embodiments, the dCas9 is a fusion protein fused to a transcriptional activator or repressor such as VP64 or KRAB, respectively.

As set forth above, each nucleic acid construct can comprise one or more expression cassettes encoding a reporter gene. Thus, a different reporter gene can be used for each construct, to individually track each nucleic acid construct in a cell or a population of cells. Cells include, but are not limited to, eukaryotic cells, animal cells, plant cells, fungal cells, and the like. Optionally, the cells are in a cell culture. Optionally, the cell is a mammalian cell, for example, a human cell. The cell can be in vitro or ex vivo. The cell can also be a primary cell, a germ cell, a stem cell or a precursor cell. The precursor cell can be, for example, a pluripotent stem cell or a hematopoietic stem cell. Introduction of the composition into cells can be cell cycle dependent or cell cycle independent. Methods of synchronizing cells to increase a proportion of cells in a particular phase are known in the art. Depending on the type of cell to be modified, one of skill in the art can readily determine if cell cycle synchronization is necessary.

The method can be performed by contacting a plurality of mammalian cells with a plurality of vectors to form a plurality of vector-infected cells. In some examples, the vectors are lentiviral vectors that are packaged into viral particles for infection of cells. The multiplicity of infection (MOI) can be controlled to ensure that the majority of the cells comprise no more than a single vector or a single integration event per cell.

In some examples, the plurality of cells is a heterogeneous population of cells (i.e., a mixture of different cells types) or a homogeneous population of cells. In some examples, the plurality contains at least two different cell types. In some examples, the cells in the plurality include healthy and/or diseased cells from a thymus, white blood cells, red blood cells, liver cells, spleen cells, lung cells, heart cells, brain cells, skin cells, pancreas cells, stomach cells, cells from the oral cavity, cells from the nasal cavity, colon cells, small intestine cells, kidney cells, cells from a gland, brain cells, neural cells, glial cells, eye cells, reproductive organ cells, bladder cells, gamete cells, human cells, fetal cells, amniotic cells, or any combination thereof.

In typical embodiments of the present methods, a site-directed nuclease is expressed in the mammalian cells. In some examples, the mammalian cells stably express a site-directed nuclease. In some examples, the site-directed nuclease is constitutively expressed. In some examples, the site-directed nuclease is under the control of an inducible promoter. In some examples, the mammalian cells are infected with a vector comprising a polynucleotide sequence encoding the site-directed nuclease prior to or subsequent to infecting the cells with the plurality of vectors. In any of the methods, the site-directed nuclease can be transiently or stably expressed in the mammalian cells. In some examples, the site-directed nuclease is encoded by an expression cassette in the cell, the expression cassette comprising a promoter operably linked to a polynucleotide encoding the site-directed nuclease. In some examples, the promoter operably linked to the polynucleotide encoding the site-directed nuclease is a constitutive promoter. In other examples, the promoter operably linked to the polynucleotide encoding the site-directed nuclease is inducible. For example, and not to be limiting, the site-directed nuclease can be under the control of a tetracycline inducible promoter, a tissue-specific promoter, or an IPTG-inducible promoter.

Once the cells have been infected, the cells are cultured for a sufficient amount of time to allow sgRNA: site-directed nuclease complex formation and transcriptional modulation, such that a pool of cells expressing a detectable phenotype can be selected from or detected among the plurality of infected cells, and/or such that the individual expression levels of target genes within cells expressing distinct sgRNAs comprising one or more mismatches and/or one or more constant region mutations can be assessed.

For example, in some embodiments, large-scale libraries can be transduced into cells, e.g., K562 CRISPRi or Jurkat CRISPRi cells, e.g., at MOI of <1 using standard methods. Following growth and appropriate selection for transduced cells, cells can be harvested, e.g., by centrifugation. In some embodiments, the genomic DNA is isolated and the sgRNA-encoding region enriched, amplified, and processed for sequencing (e.g., as disclosed in Horlbeck et al. (2016), eLife 5:e19760, the entire disclosure of which is herein incorporated by reference). The region is excised, purified, quantified, and amplified by PCR, prior to sequencing using standard methods and as described in the Examples. Phenotypes such as growth can be analyzed using known methods, e.g., by calculating the log 2 change in enrichment of an sgRNA at a given time point, subtracting the equivalent median values for all non-targeting sgRNAs, and dividing by the number of doublings in the population. The relative activities of mismatched sgRNAs, for example, can be calculated by dividing the phenotypes of the mismatched sgRNAs by those for the corresponding perfectly matched sgRNAs, e.g., as described in the Examples.

### Sequencing and Analysis

Any of a number of methods can be used to evaluate the effects of an sgRNA of the invention, e.g., to evaluate the precise expression level of a gene in the presence of the sgRNA and CRISPRi or CRISPRa, and/or to evaluate one or more phenotypes generated by the sgRNA in the presence of the CRISPRi or CRISPRa. In some embodiments, sets or libraries of sgRNAs and their effects on the transcriptome and/or other phenotypes are evaluated using Perturb-seq. In such methods, the sgRNAs are cloned into a vector such as a CROP-seq vector (as described, e.g., in Datlinger et al. (2017) Nat. Methods 14:297-301; Replogle et al. (2018) bioRxiv 503367, doi:10.1101/503367, the entire disclosures of which are herein incorporated by reference), packed into lentivirus, and transduced into cells, e.g., K562 CRISPRi cells. Following growth and appropriate selection of cells, cells are loaded onto a chip, e.g., a Chromium Single Cell 3′ V2 chip (10× Genomics) according to standard methods. The CROP-seq sgRNA barcode is then amplified by, e.g., PCR using a primer specific to the sgRNA expression cassette and a standard (e.g., P5) primer, pooled with the single cell RNA-seq libraries, and then sequenced, e.g., on a HiSeq 4000 (10× Genomics). Read counts, growth phenotypes, and relative sgRNA activities are determined using standard methods and as described in the Examples, as is Perturb-seq data analysis.

The phenotype can be, for example, cell growth, survival, or proliferation. In some embodiments, the phenotype is cell growth, survival, or proliferation in the presence of an agent, such as a cytotoxic agent, an oncogene, a tumor suppressor, a transcription factor, a kinase (e.g., a receptor tyrosine kinase), a gene (e.g., an exogenous gene) under the control of a promoter (e.g., a heterologous promoter), a checkpoint gene or cell cycle regulator, a growth factor, a hormone, a DNA damaging agent, a drug, or a chemotherapeutic. The phenotype can also be protein expression, RNA expression, protein activity, or cell motility, migration, or invasiveness. In some embodiments, the selecting of the cells on the basis of the phenotype comprises fluorescence activated cell sorting, affinity purification of cells, or selection based on cell motility.

In some embodiments, after selection of a pool of cells expressing a detectable phenotype, genomic DNA comprising the nucleic acid encoding the sgRNA is amplified by polymerase chain reaction (PCR) with a pair of primers that bracket the genomic segment comprising the nucleic acid encoding the sgRNA in each cell. In some embodiments, at least one of the PCR primers includes a sample barcode sequence that is added to the amplified DNA during amplification. The sample barcode sequence allows identification of all sequencing reads from the same sample, for example, when multiplexing multiple samples into single sequencing chip or lane.

In some embodiments, individual cells from the pool or population of cells expressing a detectable phenotype are placed into individual compartments. These compartments can be, but are not limited to, wells of a tissue culture plate (e.g., microwells) or microfluidic droplets. As used herein the term “droplet” can also refer to a fluid compartment such as a slug, an area on an array surface, or a reaction chamber in a microfluidic device, such as for example, a microfluidic device fabricated using multilayer soft lithography (e.g., integrated fluidic circuits). Exemplary microfluidic devices also include the microfluidic devices available from 10× Genomics (Pleasanton, Calif.).

In some embodiments, the cells are encapsulated in droplets. Relatively small droplets can be used in the methods provided herein. In some examples, the average diameter of the droplets may be less than about 5 mm, less than about 4 mm, less than about 3 mm, less than about 1 mm, less than about 500 micrometers, or less than about 100 micrometers. The “average diameter” of a population of droplets is the arithmetic average of the diameters of each of the droplets. In the methods provided herein, the droplets may be of the same shape and/or size, or of different shapes and/or sizes, depending on the particular application. In some examples, the individual droplets have a volume of about 1 picoliter to about 100 nanoliters.

A droplet generally includes an amount of a first sample fluid in a second carrier fluid. Any technique known in the art for forming droplets may be used. An exemplary method involves flowing a stream of the sample fluid containing the target material (e.g., cells expressing a detectable phenotype) such that the stream of sample fluid intersects two opposing streams of flowing carrier fluid. The carrier fluid is immiscible with the sample fluid. Intersection of the sample fluid with the two opposing streams of flowing carrier fluid results in partitioning of the sample fluid into individual sample droplets containing the target material. The carrier fluid may be any fluid that is immiscible with the sample fluid. An exemplary carrier fluid is oil. Optionally, the carrier fluid includes a surfactant or is a fluorous liquid. Optionally, the droplets contain an oil and water emulsion. Oil-phase and/or water-in-oil emulsions allow for the compartmentalization of reaction mixtures within aqueous droplets. The emulsions can comprise aqueous droplets within a continuous oil phase. The emulsions provided herein can be oil-in-water emulsions, wherein the droplets are oil droplets within a continuous aqueous phase.

In some embodiments, a microfluidic device is used to generate single cell droplets, for example, a single cell emulsion droplet. The microfluidic device ejects single cells in aqueous reaction buffer into a hydrophobic oil mixture. The device can create thousands of droplets per minute. In some cases, a relatively large number of droplets can be generated, for example, at least about 10, at least about 30, at least about 50, at least about 100, at least about 300, at least about 500, at least about 1,000, at least about 3,000, at least about 5,000, at least about 10,000, at least about 30,000, at least about 50,000, or at least about 100,000 droplets. In some cases, some or all of the droplets may be distinguishable, for example, on the basis of an oligonucleotide present in at least some of the droplets (e.g., which may include one or more unique sequences or barcodes). In some cases, at least about 50%, at least about 60%, at least about 70%, at least about 80%, at least about 90%, at least about 95%, at least about 97%, at least about 98%, or at least about 99% of the droplets may be distinguishable.

In some cases, after the droplets are created, the device ejects the mixture of droplets into a trough. The mixture can be pipetted or collected into a standard reaction tube for thermocycling and PCR amplification. Single cell droplets in the mixture can also be distributed into individual wells, for example, into a multiwell plate for thermocycling and PCR amplification in a thermal cycler. After amplification, the droplets can be analyzed, for example, by sequencing, to identify sgRNAs and their corresponding unique barcodes in each single cell. In some cases, the cells are lysed inside the droplet before or after amplification. In other cases, the droplets can be distributed onto a chip for amplification. Numerous methods of generating droplets and amplifying nucleic acids therein are known in the art. See, for example, Abate et al., “DNA sequence analysis with droplet-based microfluidic,” Lab Chip 13: 4864-4869 (2013); and Kaler et al. “Droplet microfluidics for Chip-Based Diagnostics,” Sensors 14(12): 23283-23306 (2014)), both of which are incorporated herein in their entireties by this reference.

Droplets containing cells may optionally be sorted according to a sorting operation prior to merging with one or more reagents (e.g., as a second set of droplets). In some embodiments, a cell can be encapsulated together with one or more reagents in the same droplet, for example, biological or chemical reagents, thus eliminating the need to contact a droplet containing a cell with a second droplet containing one or more reagents. Additional reagents may include DNA polymerase enzymes, reverse transcriptase enzymes, including enzymes with terminal transferase activity, primers, and oligonucleotides. In some embodiments, the droplet that encapsulates the cell already contains one or more reagents prior to encapsulating the cell in the droplet. In yet other embodiments, the reagents are injected into the droplet after encapsulation of the cell in the droplet. In some embodiments, the one or more reagents may contain reagents or enzymes such as a detergent that facilitates the breaking open of the cell and release of the cellular material therein. Once the reagents are added to the droplets containing the cells, the DNA comprising the nucleic acid encoding the sgRNA can be amplified in the droplet, for example, by polymerase chain reaction (PCR).

In some embodiments, after thermocycling and PCR, the amplified products can be recovered from the droplet using numerous techniques known in the art. For example, ether can be used to break the droplet and create an aqueous/ether layer which can be evaporated to recover the amplification products. Other methods include adding a surfactant to the droplet, flash-freezing with liquid nitrogen and centrifugation. Once the amplification products are recovered, the products can be further amplified and/or sequenced.

The methods provided herein comprise sequencing the amplified DNA. Sequencing methods include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, Calif.), ion semiconductor sequencing, ligation sequencing, sequencing by synthesis (Illumina, San Diego, Ca), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLiD) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term “high throughput sequencing” refers to all methods related to sequencing nucleic acids where more than one nucleic acid sequence is sequenced at a given time.

Any of the methods provided herein can optionally comprise deep sequencing of the amplified DNA. As used herein, “deep sequencing” refers to highly redundant sequencing of a nucleic acid. The redundancy (i.e., depth) of the sequencing is determined by the length of the sequence to be determined (X), the number of sequencing reads (N), and the average read length (L). The redundancy is then N×L/X. In the case of sgRNAs, the length of the sequence can be the length of the targeting sequence, the full length of the sgRNA, or the length of a portion of the sgRNA that contains the targeting sequence. The sequencing depth can be, or be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 150, 200, 300, 500, 500, 700, 1000, 2000, 3000, 4000, 5000 or more. Deep sequencing can provide an accurate number of the relative frequency of the sgRNAs. Deep sequencing can also provide a high confidence that even sgRNAs that are rarely present in a population of cells (e.g., a population of selected test cells) can be identified.

Once DNA is amplified from each cell, the nucleic acid encoding the sgRNA is sequenced from the amplified DNA. The barcode sequence provides a unique sequence for the sgRNA present in each cell. Once the cells and sgRNAs have been identified, the DNA targets of the sgRNAs can be further analyzed to determine their precise expression levels and/or how and/or to what extent the modulated expression of the DNA targets affect the phenotype.

Disclosed are materials, compositions, kits, and components that can be used for, can be used in conjunction with, can be used in preparation for, or are products of the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### 4. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. Titrating Gene Expression with Series of Systematically Compromised CRISPR Guide RNAs

### Abstract

Biological phenotypes arise from the degrees to which genes are expressed, but the lack of tools to precisely control gene expression limits our ability to evaluate the underlying expression-phenotype relationships. Here, we describe a readily implementable approach to titrate expression of human genes using series of systematically compromised sgRNAs and CRISPR interference. We empirically characterize the activities of compromised sgRNAs using large-scale measurements across multiple cell models and derive the rules governing sgRNA activity using deep learning, enabling construction of a compact sgRNA library to titrate expression of 2,400 genes involved in central cell biology and a genome-wide in silico library. Staging cells along a continuum of gene expression levels combined with rich single-cell RNA-seq readout reveals gene-specific expression-phenotype relationships with expression level-specific responses. Our work provides a general tool to control gene expression, with applications ranging from tuning biochemical pathways to identifying suppressors for diseases of dysregulated gene expression.

### Results

Mismatched sgRNAs Mediate Diverse Intermediate Phenotypes

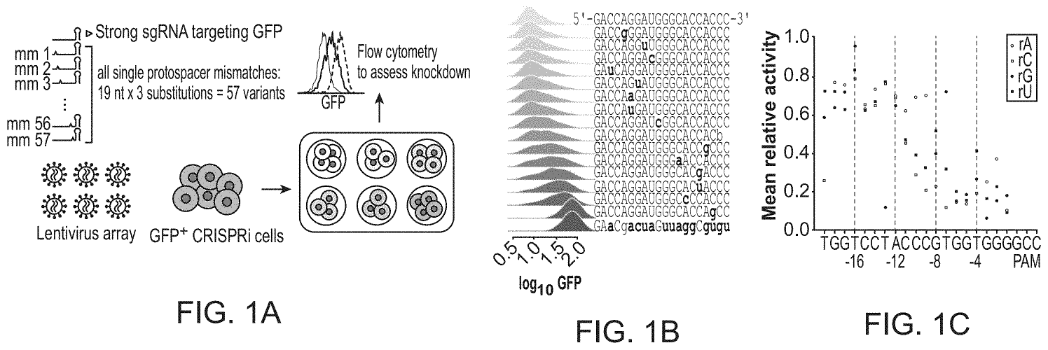

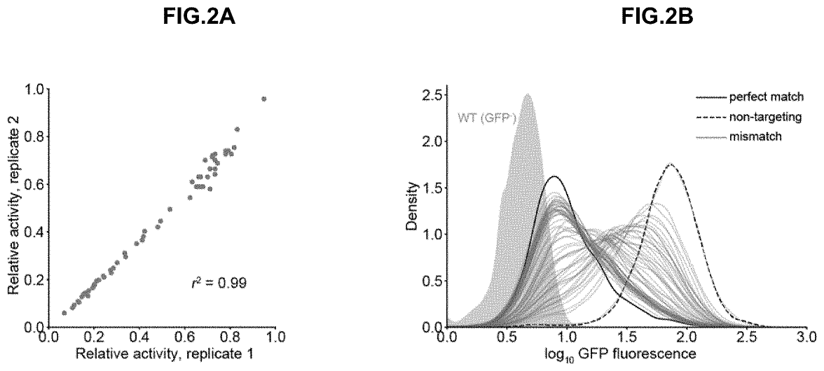

To comprehensively characterize the activities of mismatched sgRNAs in CRISPRi-mediated knockdown, we introduced all 57 singly mismatched variants of a GFP-targeting sgRNA (18) into GFP+ K562 CRISPRi cells and measured GFP levels by flow cytometry (FIG. 1A). Cells harboring mismatched sgRNAs experienced knockdown levels between those of cells with the perfectly matched sgRNA (94%) and cells with a non-targeting control sgRNA (FIGS. 1B, 2A-2B, Table 1). As expected, sgRNAs with mismatches in the PAM-proximal seed region (12,13) had strongly compromised activity. By contrast, sgRNAs with mismatches in the PAM-distal region mediated GFP knockdown to an extent similar to that of the unmodified sgRNA, albeit with substantial variability depending on the type of mismatch (FIGS. 1B-1C). The distributions of GFP levels with mismatched sgRNAs were largely unimodal, although the distributions were typically broader than with the perfectly matched sgRNA or the control sgRNA (FIGS. 1B, 2B). These results suggest that series of mismatched sgRNAs can be used to titrate gene expression at the single-cell level, but that mismatched sgRNA activity is modulated by complex factors.

Rules of Mismatched sgRNA Activity Derived from a Large-Scale Screen

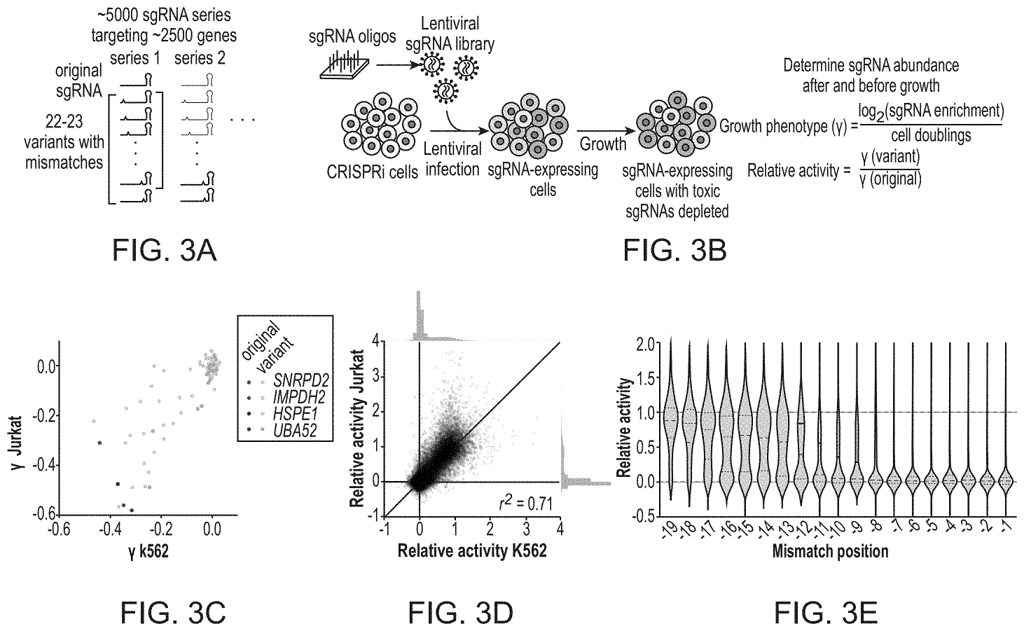

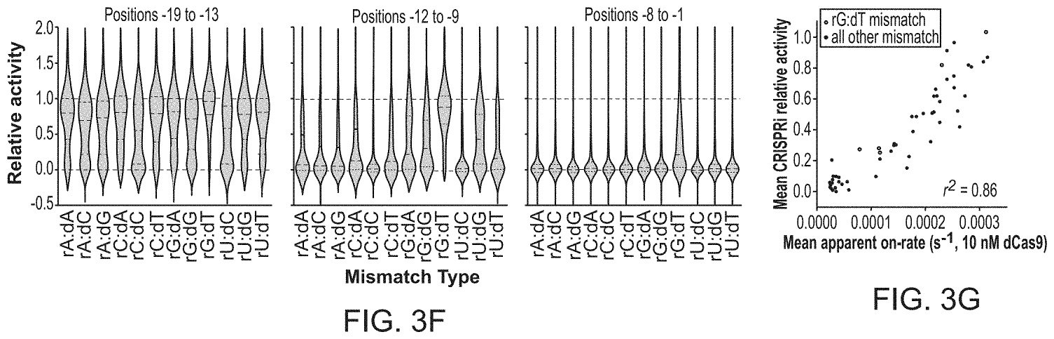

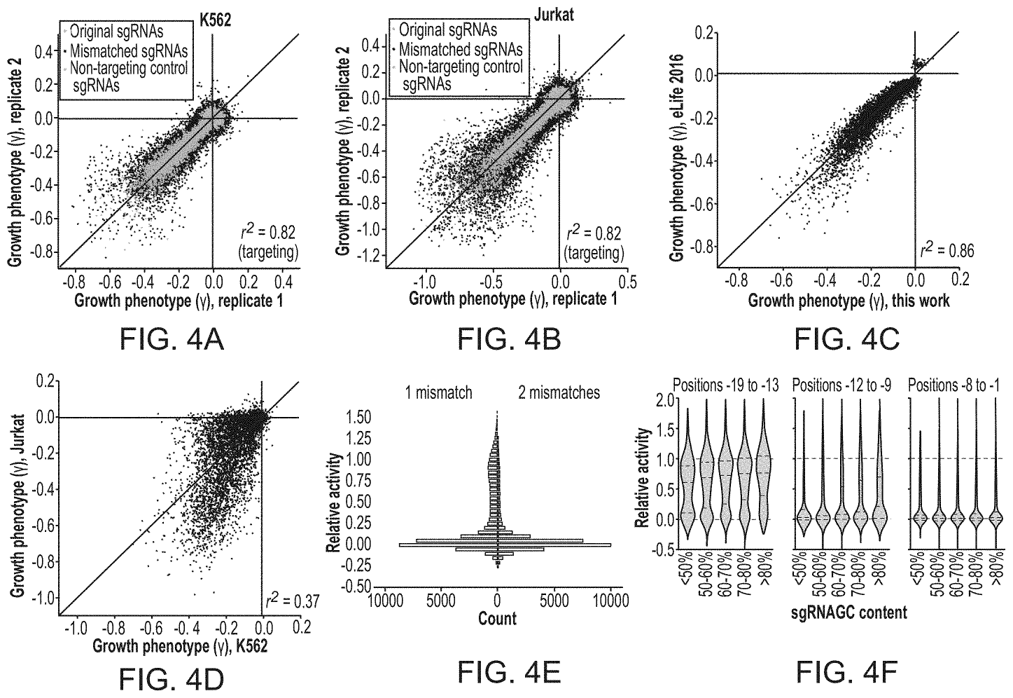

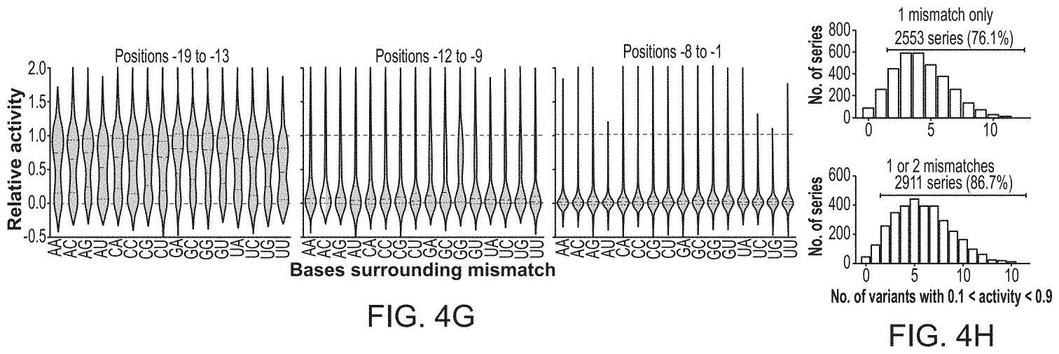

We reasoned that we could empirically derive the factors governing the influence of mismatches on sgRNA activity by measuring growth phenotypes imparted by a large number mismatched sgRNAs in a pooled screen. For this purpose, we generated a ˜120,000-element library comprising series of variants for 4,898 sgRNAs targeting 2,499 genes with growth phenotypes in K562 cells (19). Each individual series, herein referred to as an allelic series, contains the original, perfectly matched sgRNA and 22-23 variants with one or two mismatches (FIG. 3A). We then measured CRISPRi growth phenotypes (γ, for which a more negative value indicates a stronger growth defect) for each sgRNA in this library in both K562 (chronic myelogenous leukemia) and Jurkat (acute T-cell lymphocytic leukemia) cells using pooled screens (15,20) (FIGS. 3B, 4A-4D, Methods). Growth phenotypes of targeting sgRNAs were well-correlated in biological replicates (FIGS. 4A-4B, Pearson r2 [K562]=0.82; Pearson r2 [Jurkat]=0.82) and recapitulated previously reported phenotypes (19) (FIG. 4C).

Mismatched sgRNAs mediated a range of phenotypes, spanning from that of the corresponding perfectly matched sgRNA to those of negative control sgRNAs (FIG. 3C). To account for differences in absolute growth phenotypes, we normalized the phenotype of each mismatched sgRNA to that of its corresponding perfectly matched sgRNA (relative activity, FIG. 3B) and filtered for series in which the perfectly matched sgRNA had a strong growth phenotype (Methods). Relative activities measured in K562 and Jurkat cells were well-correlated (FIG. 3D, Pearson r2=0.71), regardless of differences in absolute phenotype of the perfectly matched sgRNAs (Pearson r2=0.74 for |γ[K562]−γ[Jurkat]|>0.2; Pearson r2=0.70 for |γ[K562]−γ[Jurkat]|<0.2). We therefore averaged relative activities from both cell lines for further analysis (Methods). Although the majority of mismatched sgRNAs were inactive (FIG. 3E), particularly if they contained two mismatches (FIG. 4E), a substantial fraction exhibited intermediate activity (19,596 sgRNAs with 0.1<relative activity <0.9, 25.5% of sgRNAs in series passing filter).

To understand the rules governing the impacts of mismatches on sgRNA activity, we stratified the relative activities of singly-mismatched sgRNAs by properties of the mismatch. As expected, mismatch position was a strong determinant of activity, with mismatches closer to the PAM leading to lower relative activity (FIG. 3E). In agreement with patterns of Cas9 off-target activity 21, sgRNAs with rG:dT mismatches (A to G mutations in the sgRNA) retained substantial activity even for mismatches close to the PAM (FIG. 3F). Other factors were of lower magnitude and more context-dependent, such as the associations of higher GC content with higher activity for mismatches located 9 or more bases upstream of the PAM (positions −9 to −19), and of mismatch-surrounding G nucleotides with marginally higher activity for mismatches in the intermediate region (FIGS. 4F-4G). The activities of mismatched sgRNAs thus appear to be determined by general biophysical rules; a premise further supported by the high correlation of relative activities obtained in two different cell lines (FIG. 3D) and the high correlation of mismatched sgRNA activities with previous in vitro measurements of dCas9 binding on-rates in the presence of mismatches (22) (FIG. 3G).

Finally, we evaluated the proportion of sgRNA series that provide access to a range of intermediate CRISPRi growth phenotypes for the targeted gene (relative activity between 0.1 and 0.9). When considering only singly-mismatched sgRNAs, 76.1% of series contain at least 2 sgRNAs with intermediate phenotypes, and that number rises to 86.7% when also including double mismatches (FIG. 4H). As we explored only ˜20% of possible single mismatches and <1% of possible double mismatches, it is likely that intermediate-activity sgRNAs also exist for the remaining series. Altogether, these results suggest that systematically mismatched sgRNAs provide a general method to titrate CRISPRi activity and, consequently, target gene expression.

Controlling sgRNA Activity with Modified Constant Regions

We also explored the orthogonal approach of generating intermediate-activity sgRNAs through modifications to the sgRNA constant region, which is required for binding to Cas9. Although previous work has established that such modifications can lead to increases or decreases in Cas9 activity or have no measurable impact (16, 23-27), the mutational landscape of the constant region has only been sparsely explored, and largely with the goal of preserving sgRNA activity.

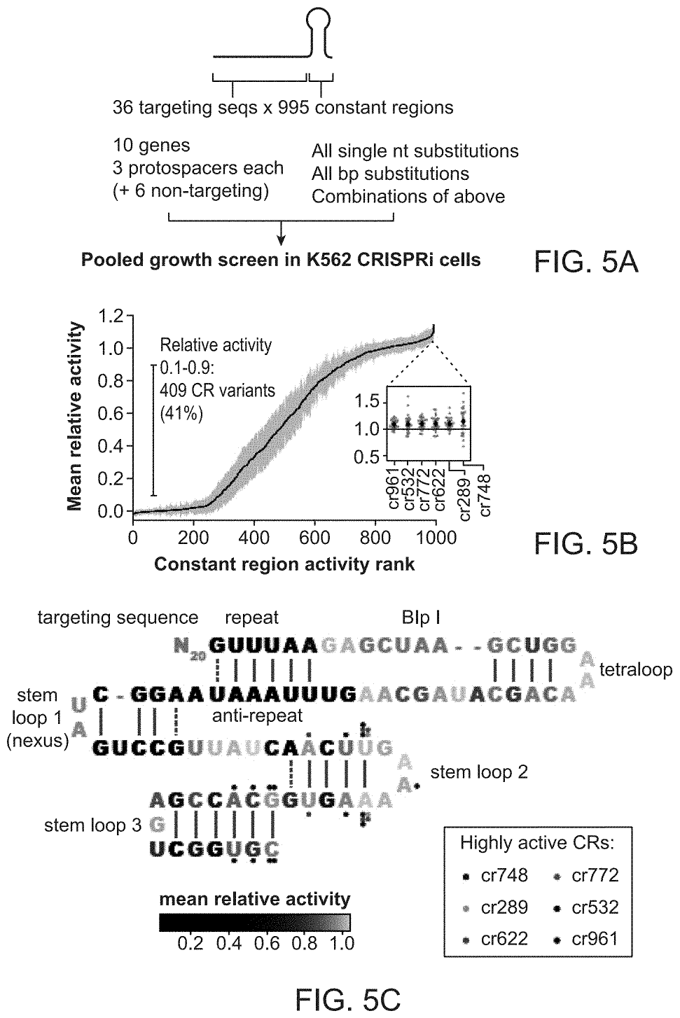

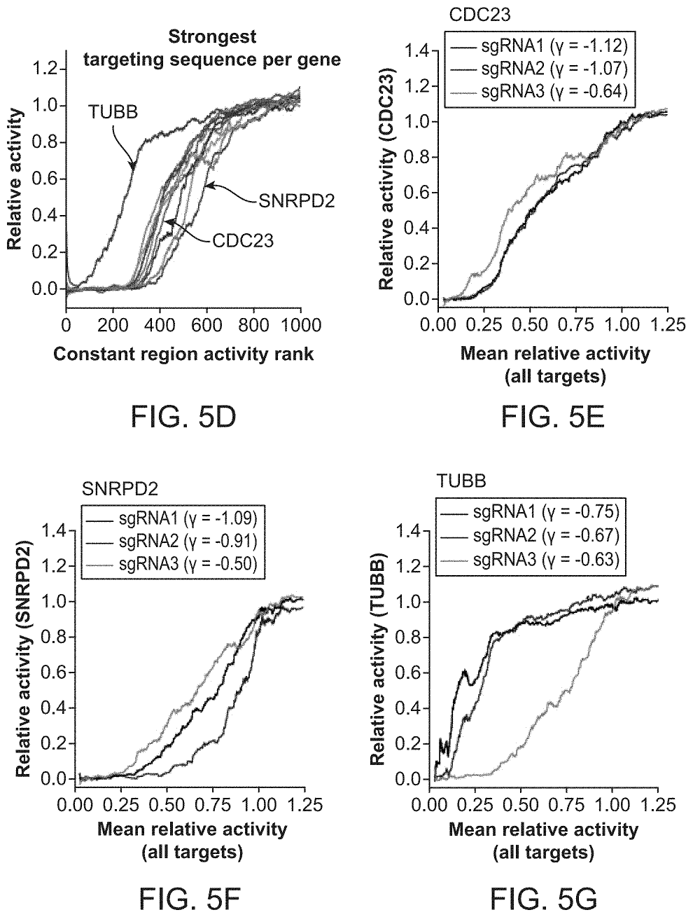

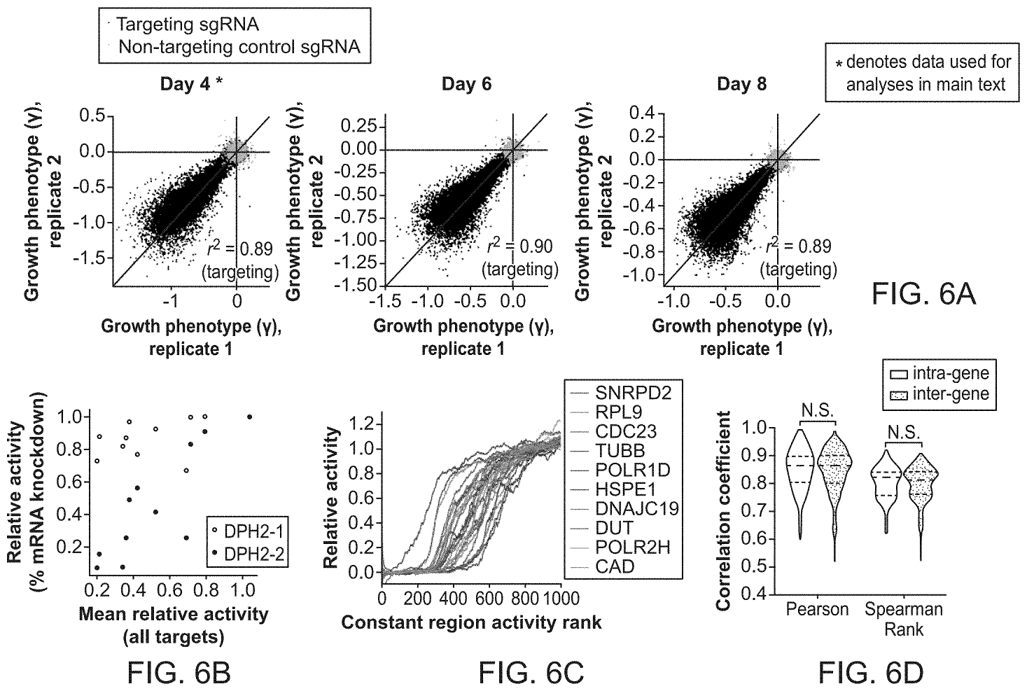

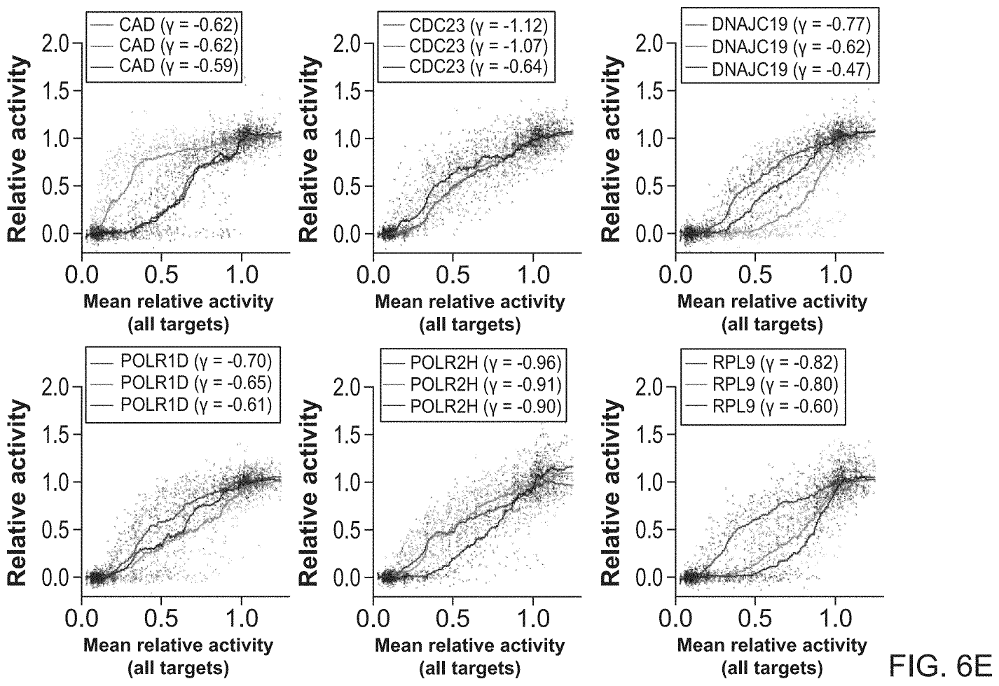

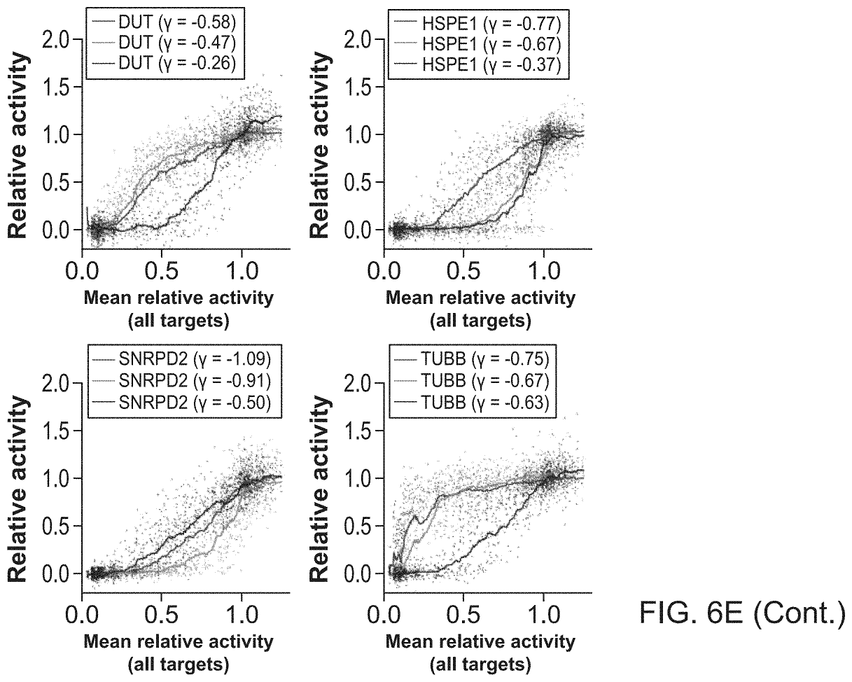

To comprehensively assess the activities of modified sgRNA constant regions, we designed a library of 995 constant region variants comprising all possible single nucleotide substitutions, base pair substitutions, and combinations of these changes (Methods, Table 6) and determined the growth phenotypes for each variant paired with 30 different targeting sequences against 10 essential genes in a pooled screen in K562 cells (FIGS. 5A, 6A; Table 6, which shows the ranking of each constant region variant in terms of its relative CRISPRi and CRISPRa activity). We calculated relative activities for each targeting sequence:constant region pair by normalizing its phenotype to that of the targeting sequence paired with the unmodified constant region, identifying 409 constant region variants that on average conferred intermediate activity (0.1-0.9, FIG. 5B). Ten variants selected for individual evaluation also mediated intermediate levels of mRNA knockdown (FIG. 6B). Mapping the activities of constant region variants with single base substitutions onto the structure recapitulated known relationships between constant region structure and function (FIG. 5C). For example, mutation of bases known to mediate contacts (16) with Cas9 (e.g. the first stem loop or the nexus) generally reduced activity, whereas mutations in regions not contacted by Cas9 (e.g. the hairpin region of stem loop 2) were well-tolerated (FIG. 5C). Notably, several variants carrying mutations in stem loop 2 had consistently increased activities and thus could be useful tools for future applications (FIGS. 5B-5C).

Evaluating the relative activities of constant region variants across different targeting sequences revealed consistent rank ordering but substantial variation in the actual values (FIGS. 5D, 6C). For example, a targeting sequence against TUBB retained high activity with ˜100 constant region variants that otherwise abolished activity for other targeting sequences, whereas a targeting sequence against SNRPD2 lost activity with ˜50 variants that otherwise conferred intermediate activity (FIG. 5D). In some but not all (FIG. 5E) cases, this heterogeneity extended to different targeting sequences against the same gene, both at the level of growth phenotype (FIGS. 5F-5G, 6D-6E) and mRNA knockdown (FIG. 6B). These differences between targeting sequences could be a consequence of specific targeting sequence:constant region structural interactions or of differences in basal sgRNA expression levels such that lowly expressed sgRNAs are more susceptible to constant region modifications. Thus, although modified constant regions can be used to titrate gene expression, the activity of a given constant region variant for a given targeting sequence is difficult to predict. We therefore focused on sgRNAs with mismatches in the targeting region for the remainder of our work, given that the activities of these sgRNAs were governed by biophysical principles, which should be more predictable.

A Neural Network Predicts Mismatched sgRNA Activities with High Accuracy

We next sought to leverage our large-scale data set of mismatched sgRNA activities to learn the underlying rules in a principled manner and to enable predictions of intermediate-activity sgRNAs against other genes. We reasoned that a convolutional neural network (CNN) would be well-suited to uncovering these rules due to the ability of CNNs to learn complex global and local dependencies on spatially-ordered features such as nucleotide sequences (28), including factors governing guide RNA activity in orthogonal CRISPR systems (29,30).

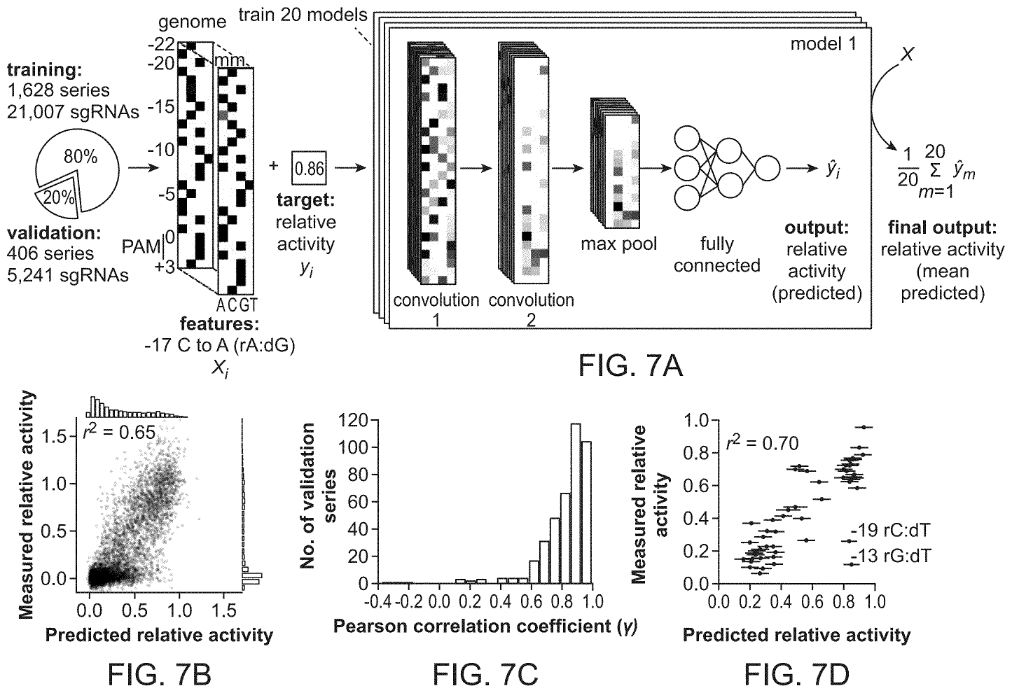

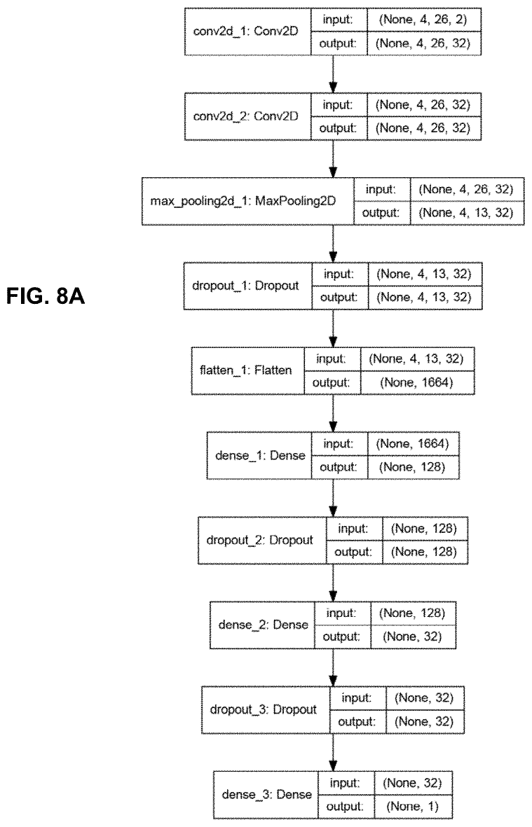

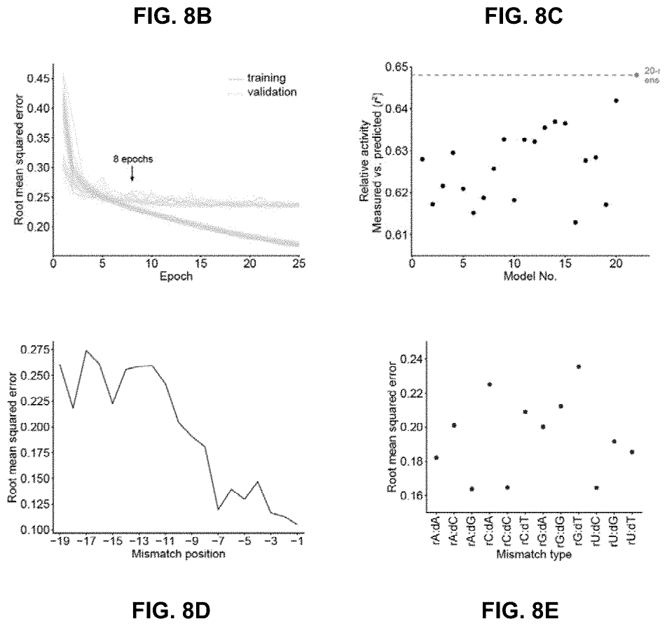

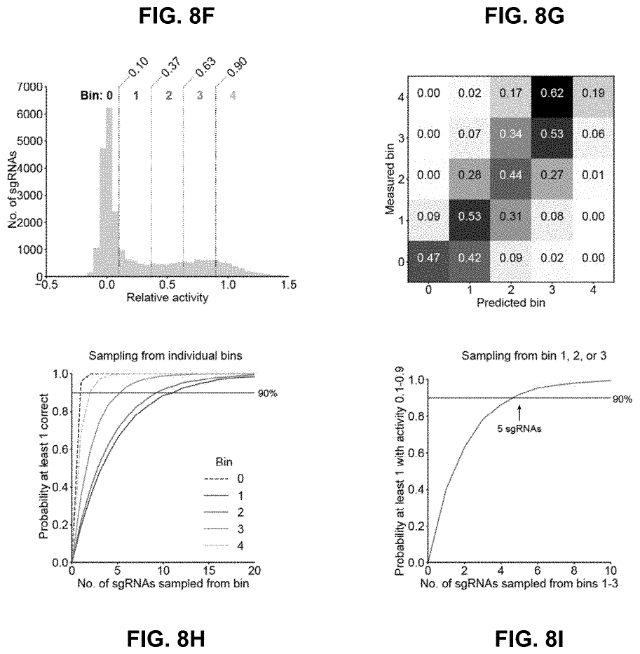

To develop a CNN model capable of predicting mismatched sgRNA activities, we constructed a model consisting of two convolution steps, a pooling step, and a 3-layer fully connected neural network (FIGS. 7A, 8A). As inputs, the model received sgRNA relative activities paired with nucleotide sequences represented by binarized 3D arrays denoting the genomic sequence of the target and the associated sgRNA mismatch (FIG. 7A). After optimizing hyperparameters using a randomized grid search, we trained 20 independent, equivalently initialized models on the same set of randomly selected sgRNAs (80% of all series) for 8 epochs, which minimized loss without extensive over-fitting (FIG. 8B). Predicted and measured sgRNA relative activities for the validation sgRNA set (i.e., the remaining 20% of series that were not used to train the model) were well-correlated (Pearson r2=0.65), with mean predictions of the 20-model ensemble outperforming all individual models (FIGS. 7B, 8C). The distribution of correlation coefficients for individual sgRNA series was unimodal with Pearson r values in the 25th-75th percentile ranging from 0.77 to 0.93, indicating that the model performed comparably well for most series (FIG. 7C). Model accuracy varied by mismatch position and type, with the highest accuracies corresponding to mismatches in the PAM-proximal seed region (FIGS. 8D-8E). Despite the fact that the model was trained on relative growth phenotypes, it also accurately predicted relative fluorescence values measured in the GFP experiment (FIG. 7D), further supporting the hypothesis that relative growth phenotypes report on biophysical attributes of specific sgRNA:DNA interactions.

To derive intermediate-activity sgRNAs for all human genes, we used the CNN ensemble to predict relative activities for all 57 singly-mismatched sgRNAs for the top 5 sgRNAs against each gene in the hCRISPRi-v2.1 library (19). Based on the accuracy of predictions for the validation set, we estimate that for any given gene, sampling 5 sgRNAs with predicted intermediate relative activity (0.1-0.9) will yield at least one sgRNA in that activity range over 90% of the time (FIGS. 8F-8I). This resource should therefore enable titrating the expression of any gene(s) of interest.

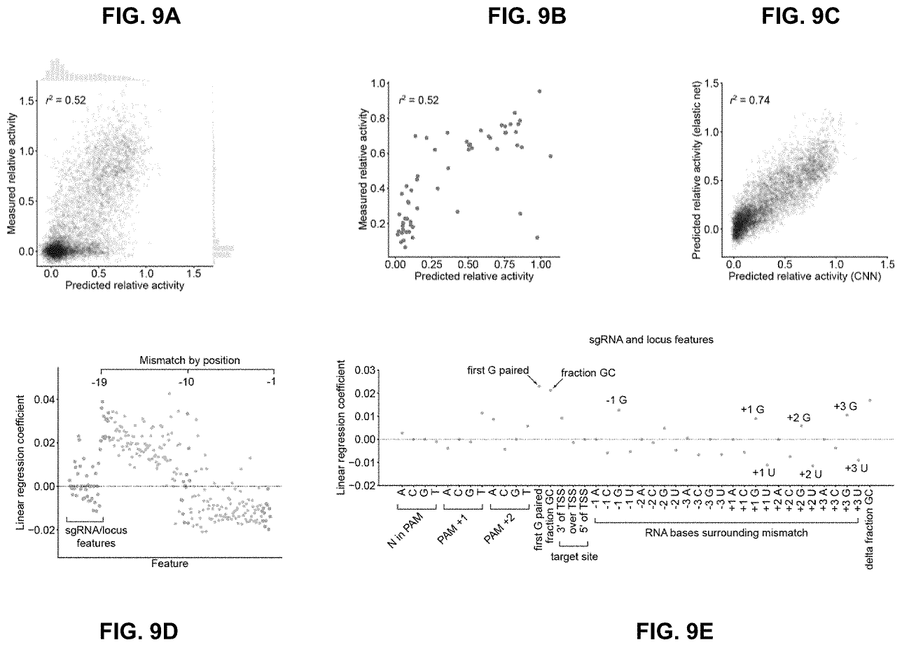

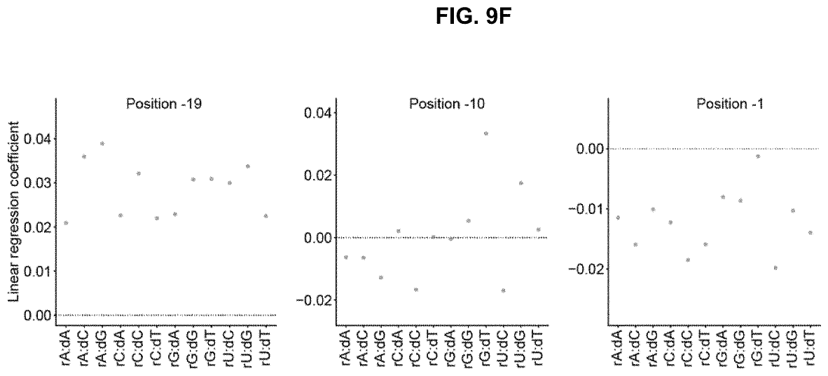

Finally, we sought to further understand the features of mismatched sgRNAs that contribute most to their activity. As the contributions of individual features in a deep learning model are difficult to assess directly, we also trained an elastic net linear regression model on the same data using a curated set of features. This linear model explained less variance in relative activities than the CNN model (r2=0.52, FIGS. 9A-9B), implying that our feature set was incomplete and/or sgRNA activity is partly determined by non-linear combinations of features; nonetheless, the relative activities predicted by the different models were well-correlated (r2=0.74, FIG. 9C). Consistent with our earlier observations, mismatch position and type were assigned the largest absolute weights in the model, although other features such as GC content in the sgRNA and the identities of flanking bases up to 3 nucleotides away from the mismatch were heavily weighted as well (FIGS. 9D-9E). For any given position, the type of mismatch contributed differentially to the prediction, with the largest variation between types occurring in the intermediate region of the targeting sequence (FIG. 9F). Taken together, these data demonstrate that the activities of mismatch-containing sgRNAs are determined by multiple factors which can be captured using supervised machine learning approaches.

A Compact Mismatched sgRNA Library Conferring Intermediate Growth Phenotypes

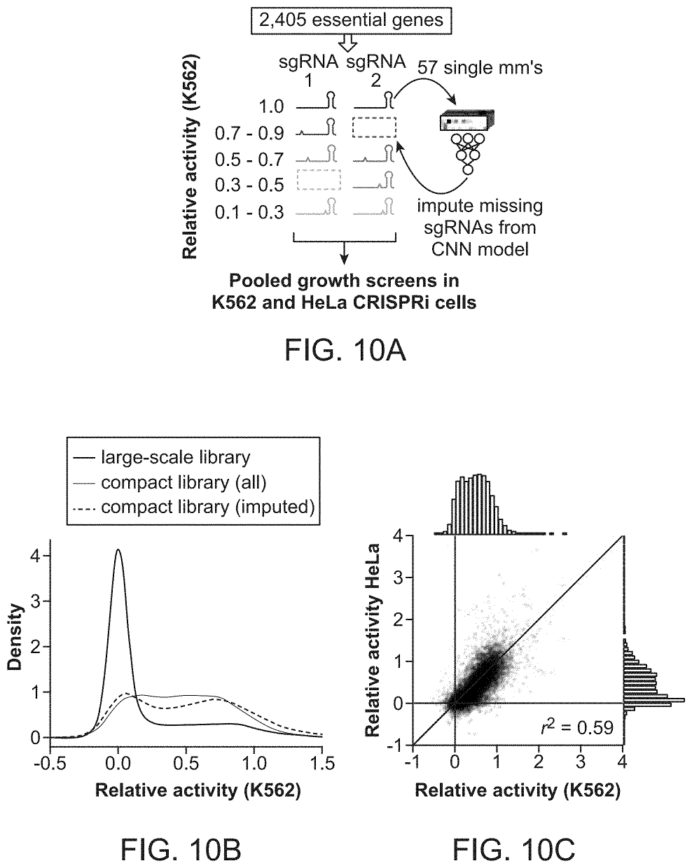

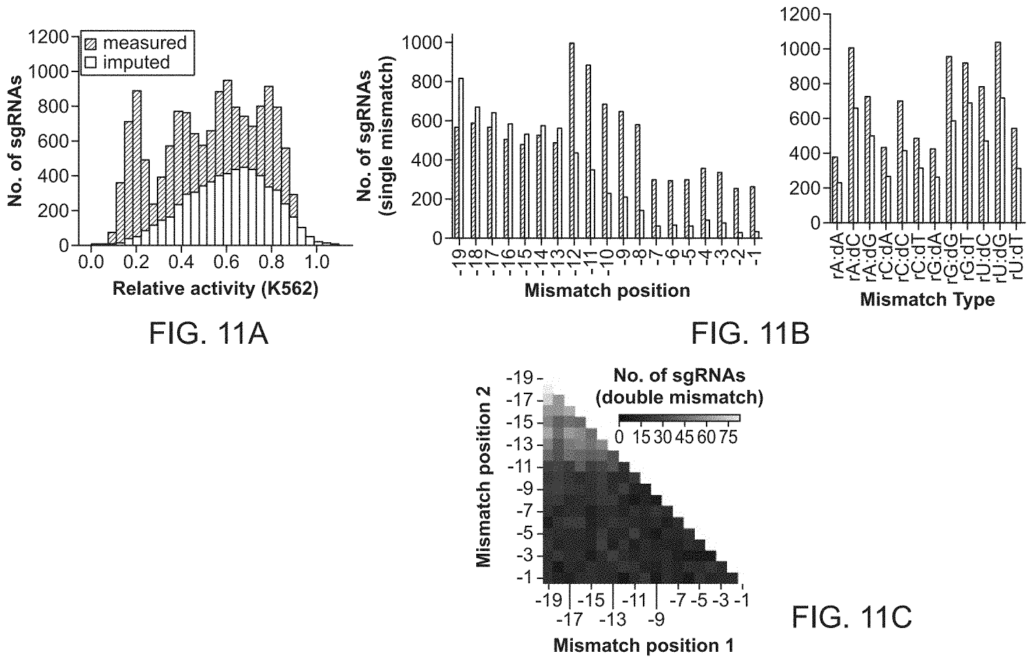

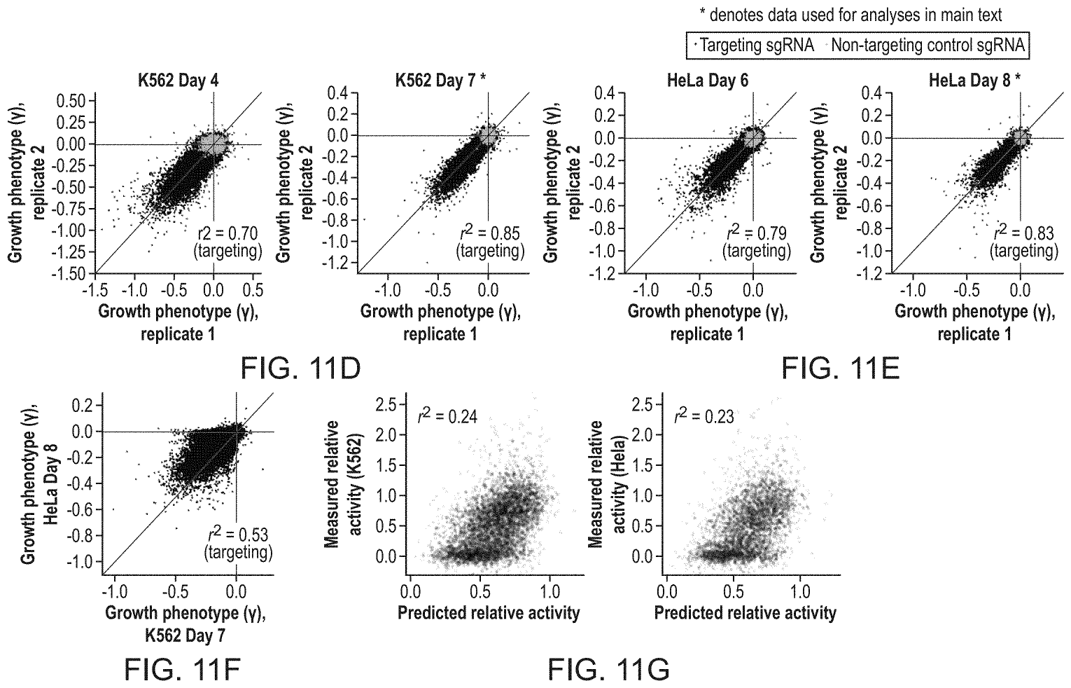

We next set out to design a more compact version of our large-scale library to titrate essential genes with a limited number of sgRNAs. We selected 2,405 genes which we had found to be essential for robust growth in K562 cells in our large-scale screen, divided the relative activity space into six bins, and attempted to select mismatched variants from each of the center four bins (relative activities 0.1-0.9) for two sgRNA series targeting each gene. If a bin did not contain a previously measured sgRNA, we selected one from the CNN model ensemble predictions (FIG. 10A), filtered to exclude sgRNAs with off-target binding potential. For each gene, 2 perfectly matched and 8 mismatched sgRNAs were selected, with approximately 32% of mismatched sgRNAs imputed from the CNN model (FIGS. 11A-11C).

We evaluated the relative activities of sgRNAs in the compact library using pooled CRISPRi growth screens in K562 and HeLa (cervical carcinoma) cells. Growth phenotypes were well-correlated in biological replicates from samples harvested at different time points after t0 in both cell lines (FIGS. 11D-11F). The CNN model predicted imputed sgRNA activities with lower accuracy than the large-scale validation (FIG. 11G), although we note that imputed sgRNAs were highly enriched in PAM-distal mutations which are associated with higher model errors (FIGS. 11B, 8E). Whereas the majority of mismatched sgRNAs in the large-scale screen had little to no activity, relative activities in the compact library were evenly distributed, ranging from inactive to full activity (FIG. 10B). Relative sgRNA activities were also well-correlated between K562 and HeLa cells (r2=0.58, FIG. 10C), suggesting that our library provides access to intermediate phenotypes for this core set of genes in multiple cell types.

To explore the utility of our compact library for chemical-genetic screens, we carried out a screen in K562 cells for sensitivity to lovastatin, a potent HMG-CoA reductase inhibitor (FIG. 11J). We hypothesized that even moderate knockdown of the direct target might significantly sensitize cells to the drug, which would lead to a unique signature when comparing growth phenotypes in drug-treated and untreated cells (τ and γ, respectively). Indeed, sgRNAs targeting HMGCR strongly reduced growth in the presence of lovastatin, and a linear regression of the HMGCR series on a τ vs. γ plot yielded one of the largest slopes of all series (FIG. 11K), demonstrating the potential to identify drug-gene interactions using this approach.

Exploring Expression Phenotype Relationships with sgRNA Series

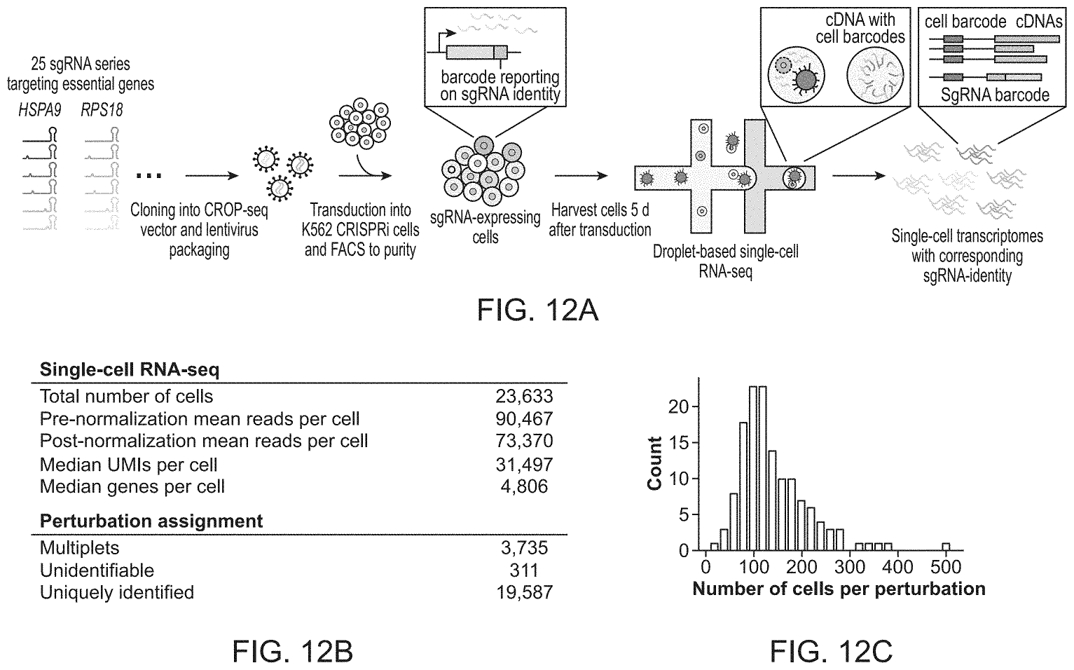

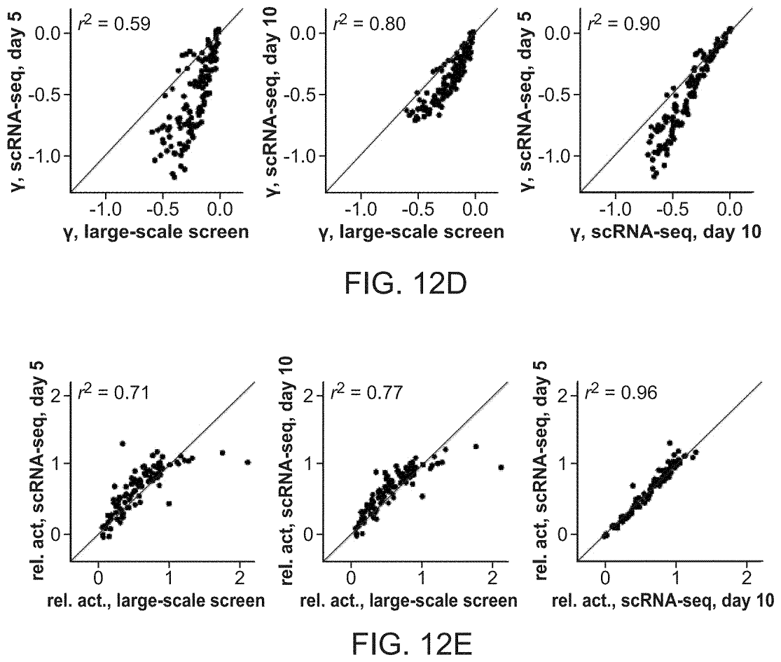

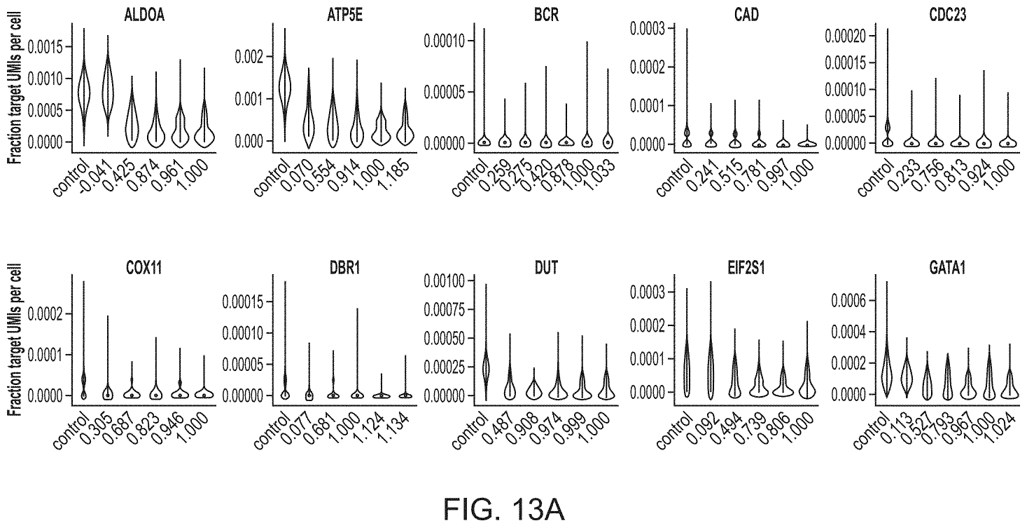

Finally, we sought to use intermediate-activity sgRNAs to explore relationships between expression levels of various genes and the resulting cellular phenotypes. To simultaneously measure gene expression levels and obtain rich phenotypes for a variety of sgRNA series, we used Perturb-seq, an experimental strategy that enables matched capture of the transcriptome and the identity of an expressed sgRNA for each individual cell in pools of cells (27, 31-33) (FIG. 12A). We chose 25 essential genes involved in diverse cell biological processes (Table 2), targeting each with a perfectly matched sgRNA and 4-5 variants with intermediate growth phenotypes (138 sgRNAs total including 10 non-targeting controls, Table 1). We then subjected pooled K562 CRISPRi cells expressing these sgRNAs from a modified CROP-seq vector 33,34 to single-cell RNA-seq (scRNA-seq), using the co-expressed sgRNA barcodes to assign unique sgRNA identities to 19,600 cells (median 122 cells per sgRNA, FIGS. 12B-12C). In addition to the single-cell transcriptomes, we measured bulk growth phenotypes conferred by sgRNAs in these cells. These growth phenotypes were well-correlated with those from the large-scale screen and were used to assign sgRNA relative activities for further analysis (Methods, FIGS. 12D-12E, Tables 3, 4).

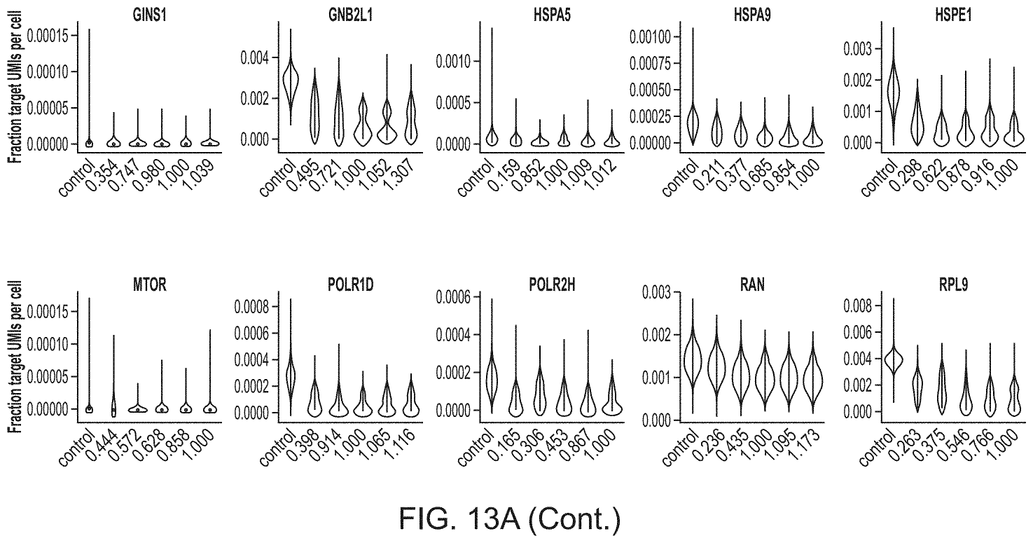

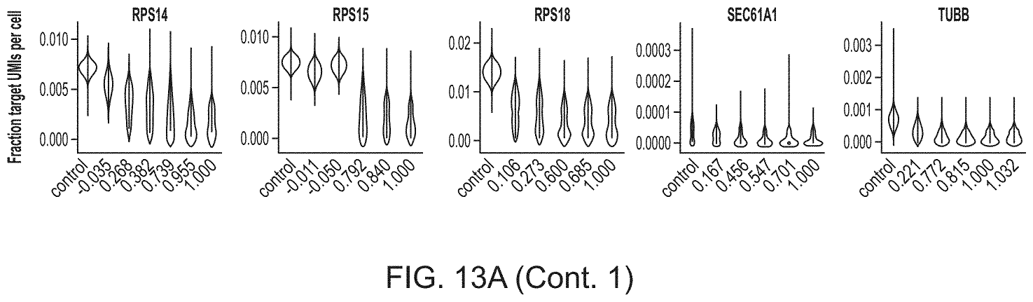

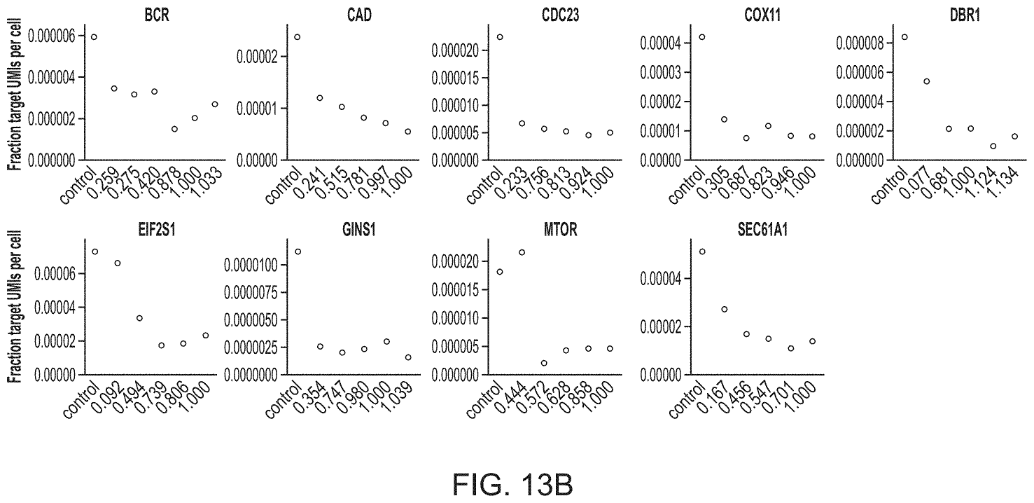

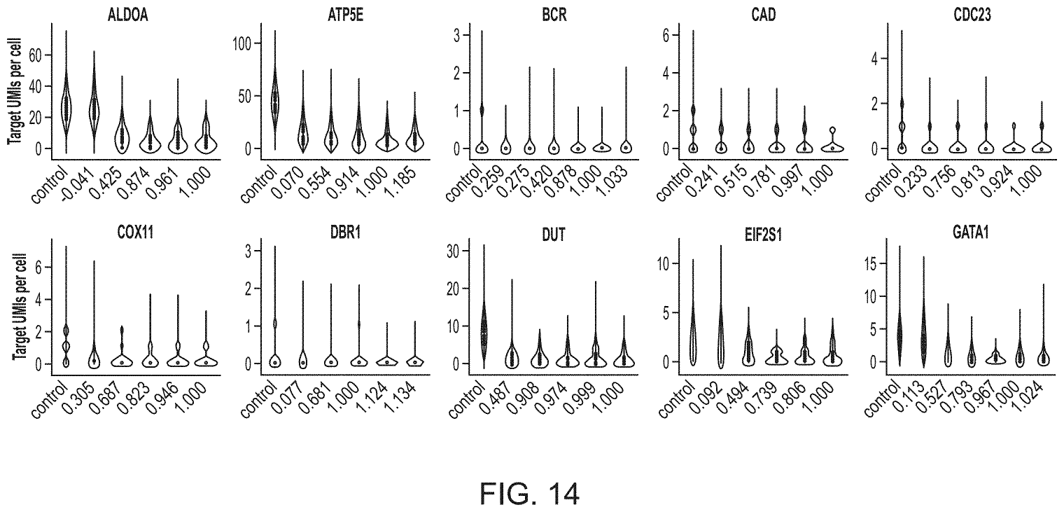

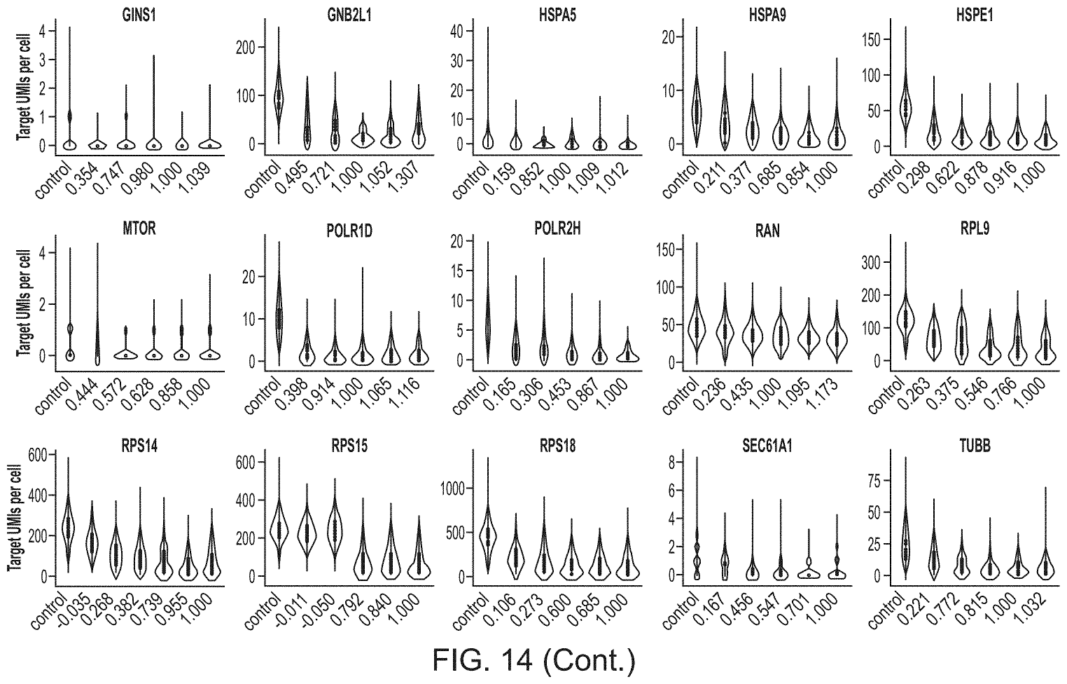

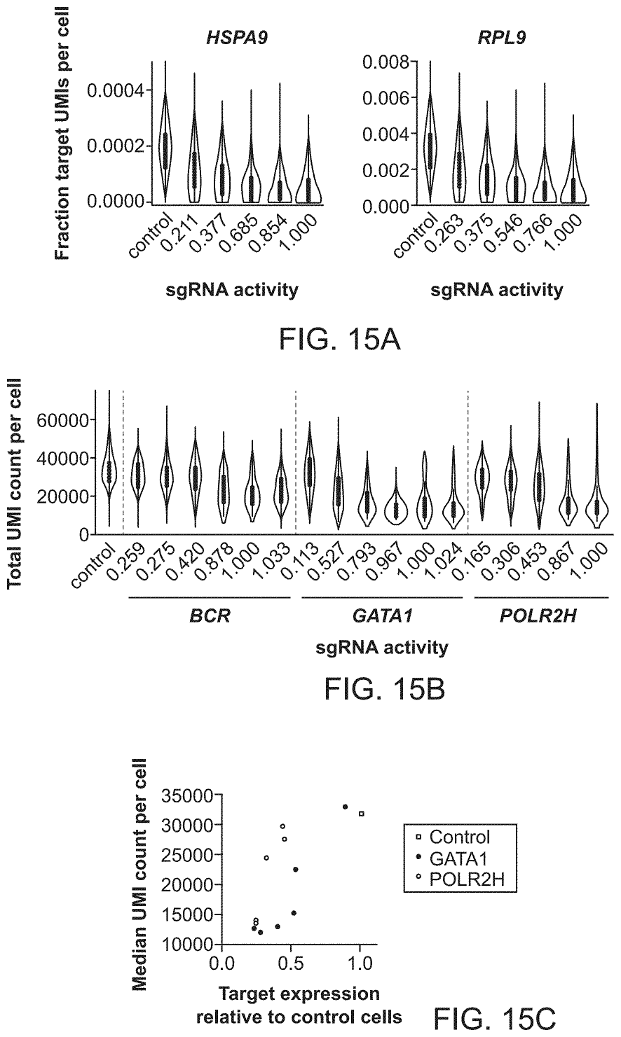

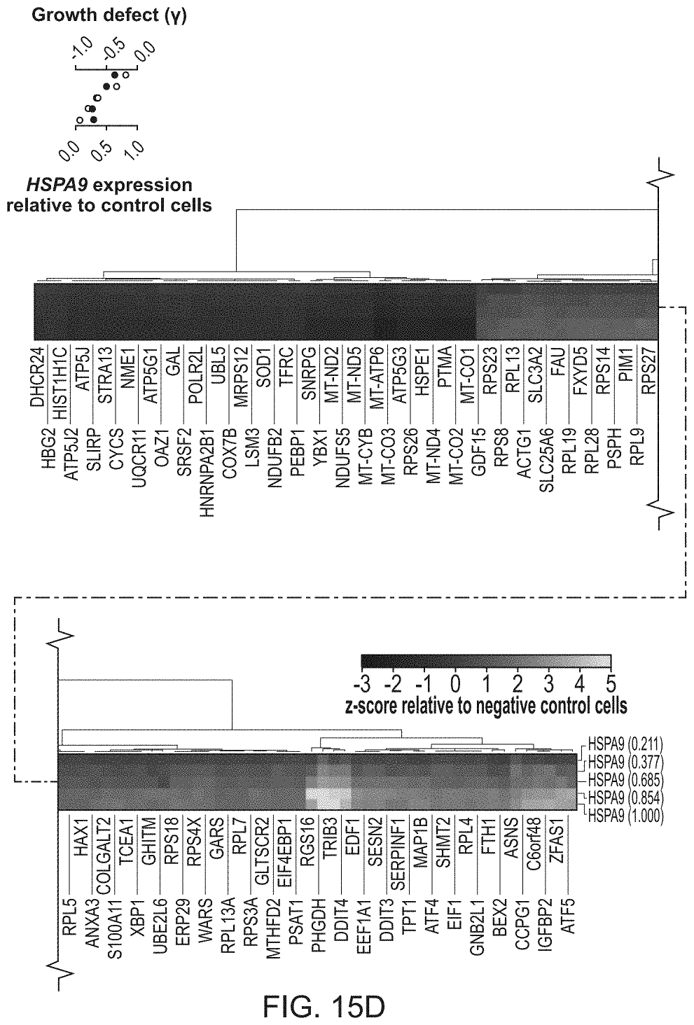

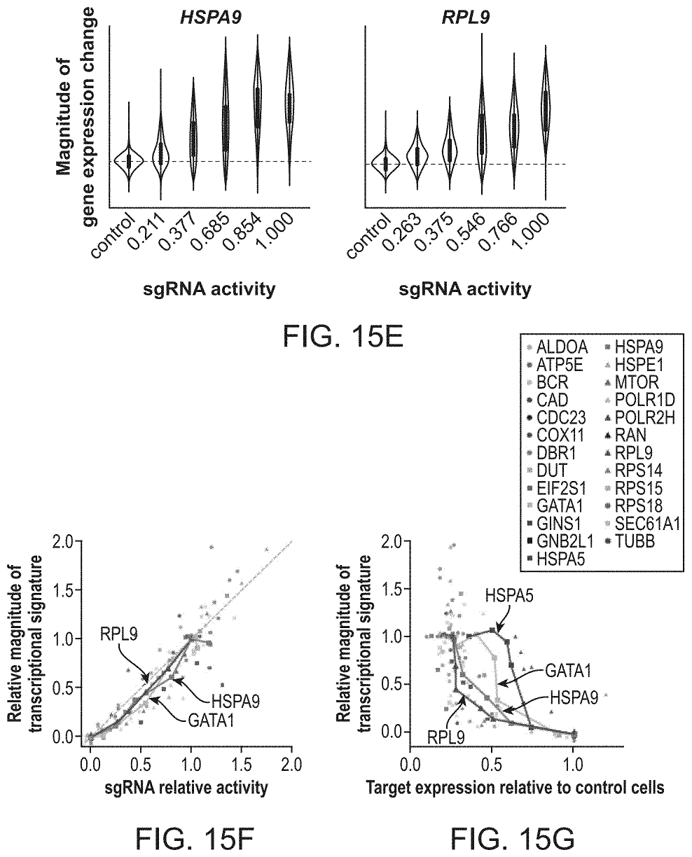

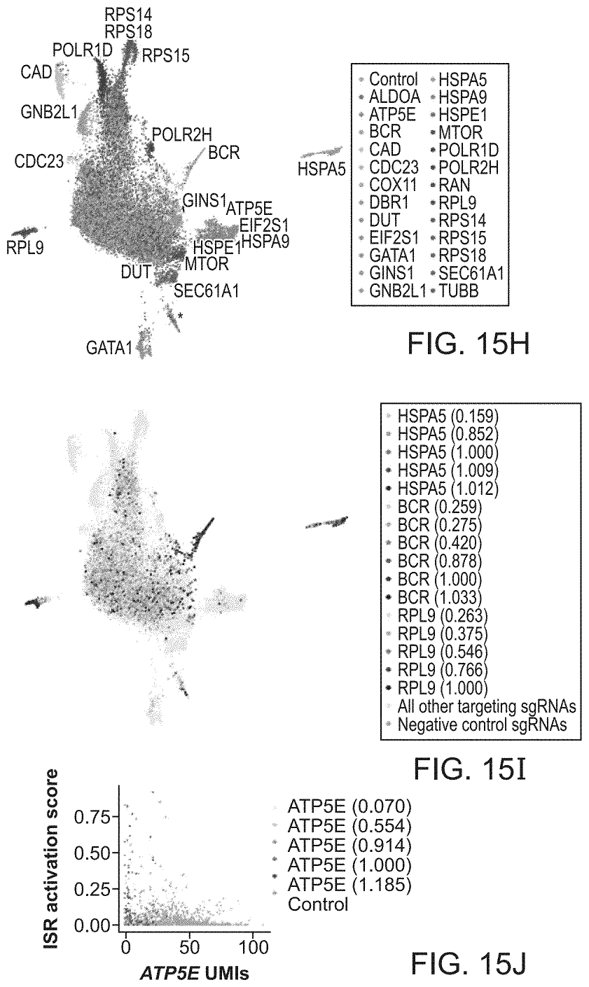

We first used the scRNA-seq data to assess the expression of the gene targeted by each sgRNA series. To account for cell-to-cell variability in transcript capture efficiency, we quantified target gene UMIs as a fraction of total UMIs in a given cell (FIG. 13), although analyzing raw UMI counts yielded similar results (FIG. 14). Approximately half of the genes we targeted were highly expressed (median >10 UMIs per cell), allowing us to directly measure target gene expression levels on the single-cell level (FIGS. 15A, 13). These distributions are largely unimodal, with medians shifting downwards with increasing sgRNA activity (FIG. 15A). For some of these genes, however, two populations with different knockdown levels are apparent (FIGS. 15A, 13A). These populations are present both with intermediate-activity sgRNAs and the perfectly matched sgRNAs, suggesting that they are not a consequence of limited knockdown penetrance for intermediate-activity sgRNAs. Owing to the limited capture efficiency of scRNA-seq, for genes with intermediate to low expression such as CAD and COX11 we typically observed 0-4 UMIs per cell, rendering the quantification of single-cell expression levels more difficult. We nonetheless observe a shift of the distribution to lower UMI numbers with increasing sgRNA activity (FIGS. 13A, 14) as well as a decrease in mean expression levels when averaging expression across all cells with the same sgRNA (FIG. 13B).

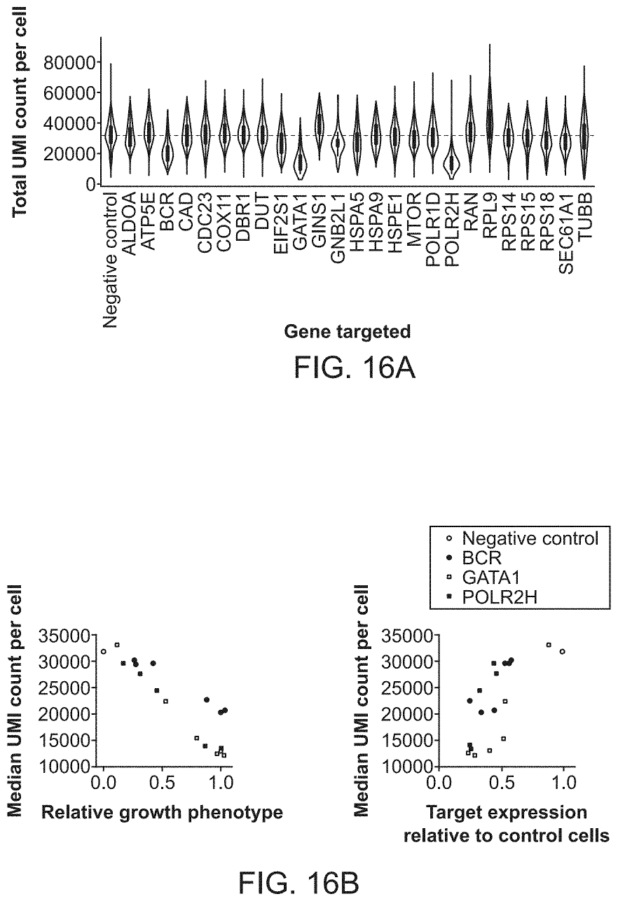

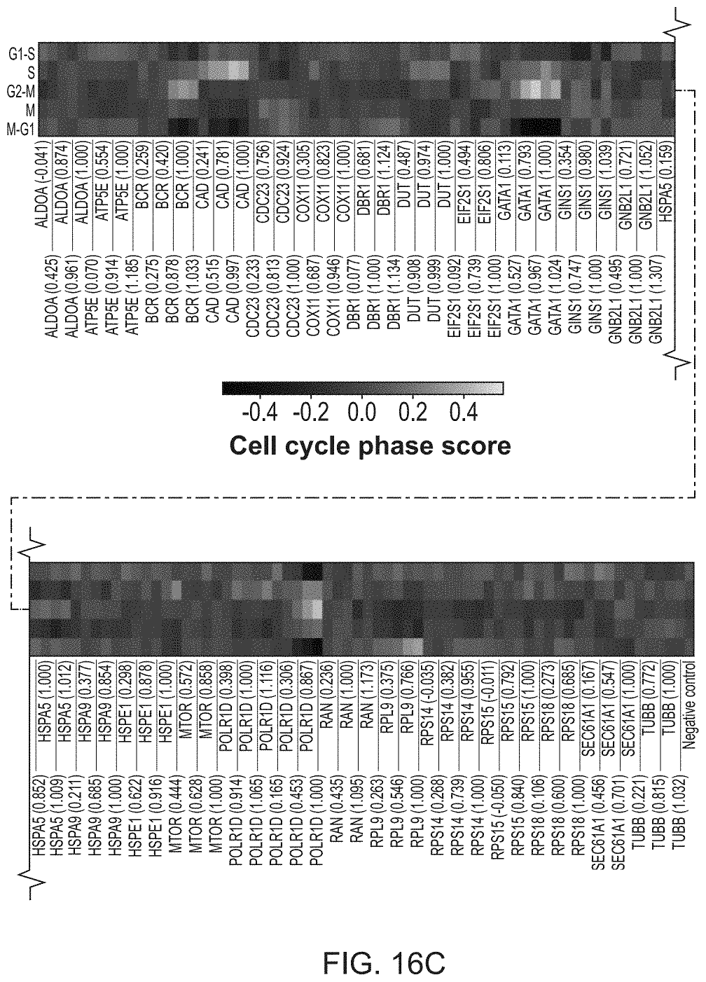

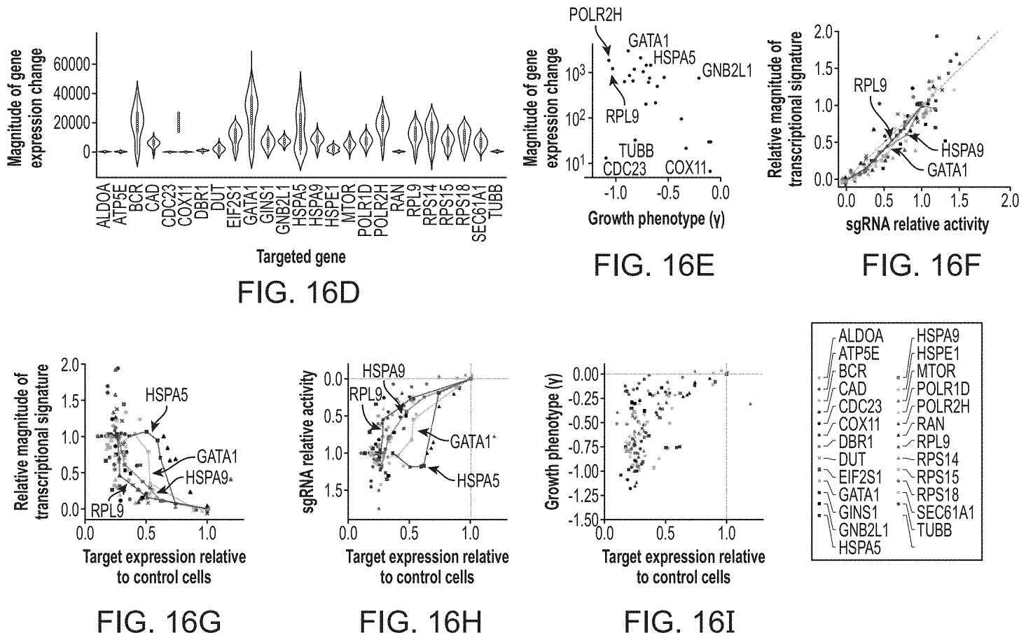

Titration is also apparent at the level of the transcriptional responses, which provides a robust single-cell measurement of the phenotype induced by depletion of the targeted gene. In the simplest cases, knockdown led to substantial reductions in cellular UMI counts, consistent with large-scale inhibition of mRNA transcription (FIGS. 15B, 16A). Examples include GATA1, a central myeloid lineage transcription factor, POLR2H, a core subunit of RNA polymerase II (as well as RNA polymerases I and III), or to a lesser extent BCR, which is fused to the driver oncogene ABL1 in K562 cells (35,36). Notably, this effect correlates linearly with growth phenotype for intermediate activity sgRNAs (FIGS. 15B, 16B) but exhibits non-linear relationships with target gene knockdown at least in the cases of GATA1 and POLR2H (FIGS. 15C, 16B, BCR levels are difficult to quantify accurately). Both relationships appear to be sigmoidal but with different thresholds: whereas cellular UMI counts drop rapidly once GATA1 mRNA levels are reduced by 50%, a larger reduction of POLR2H mRNA levels is required to achieve a similarly sized effect. Knockdown of most other targeted genes did not perturb total UMI counts to the same extent (FIG. 16A) but resulted in other transcriptional responses. Knockdown of CAD, for example, triggered cell cycle stalling during S-phase, as had been observed previously (27), with a higher frequency of stalling with increasing sgRNA activity (FIG. 16C). By contrast, knockdown of HSPA9, the mitochondrial Hsp70 isoform, induced the expected transcriptional signature corresponding to activation of the integrated stress response (ISR) including upregulation of DDIT3 (CHOP), DDIT4, ATF5, and ASNS (27,37). The magnitude of this transcriptional signature increased with increasing sgRNA activity on both the bulk population (FIG. 15D) and single-cell levels (FIG. 15E), although populations with intermediate-activity sgRNAs had larger cell-to-cell variation in the magnitudes of transcriptional responses. Similarly, the transcriptional responses to knockdown of other genes (FIG. 16D) scaled with sgRNA activity and exhibited larger variance for intermediate-activity sgRNAs (FIG. 15E).

We next explored expression-phenotype relationships in these data. Within each series, two major metrics of phenotype, bulk population growth phenotype and transcriptional response, appear to be well-correlated, despite substantial differences in the absolute magnitudes of the transcriptional responses with different series (FIGS. 15F, 16D-16F). By contrast, the relationships between either metric of phenotype and target gene expression are strongly gene-specific (FIGS. 15G, 16G-16I). For HSPAS and GATA1, for example, a comparably small reduction in mRNA levels (˜50%) was sufficient to induce a near-maximal transcriptional response and growth defect, whereas for most other genes a larger reduction was required. These results prompt the hypothesis that K562 cells are intolerant to moderate decreases in expression of GATA1 and HSPAS, with sharp transitions from growth to death once expression levels drop below a threshold. More broadly, these results highlight the utility of titrating gene expression to systematically map expression-phenotype relationships and quantitatively define gene expression sufficiency.

### Following Single-Cell Trajectories Along a Continuum of Gene Expression Levels

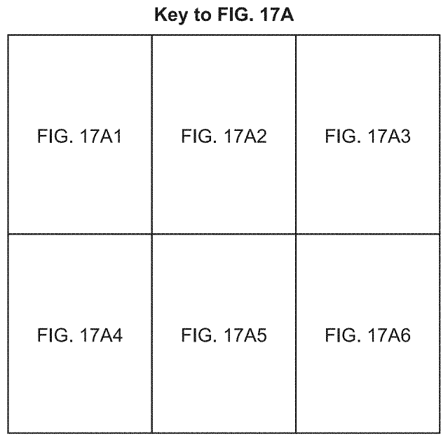

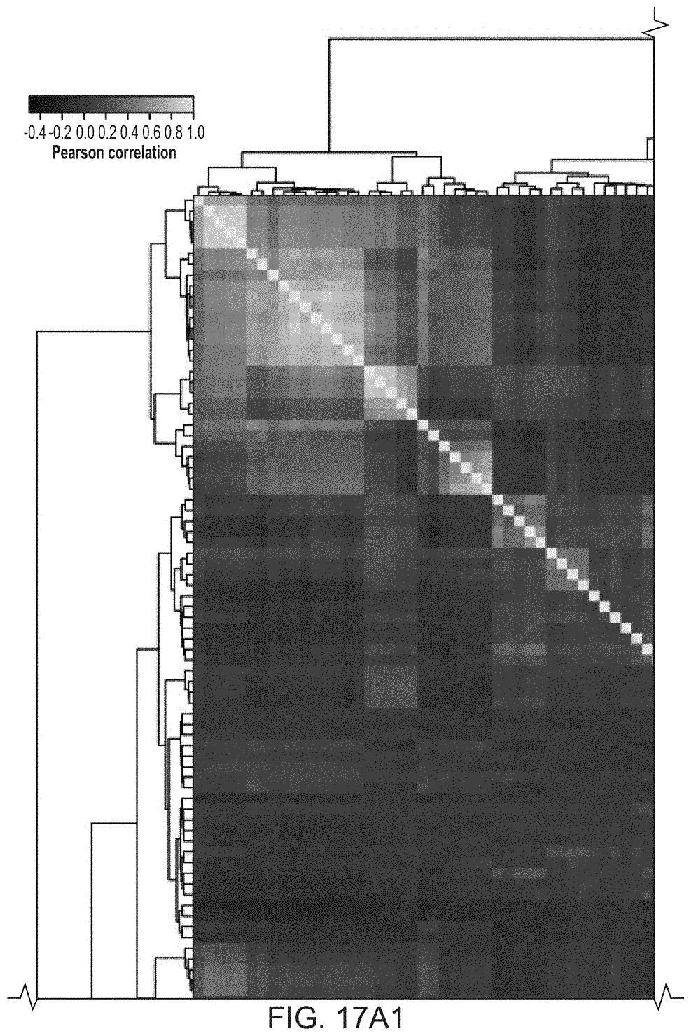

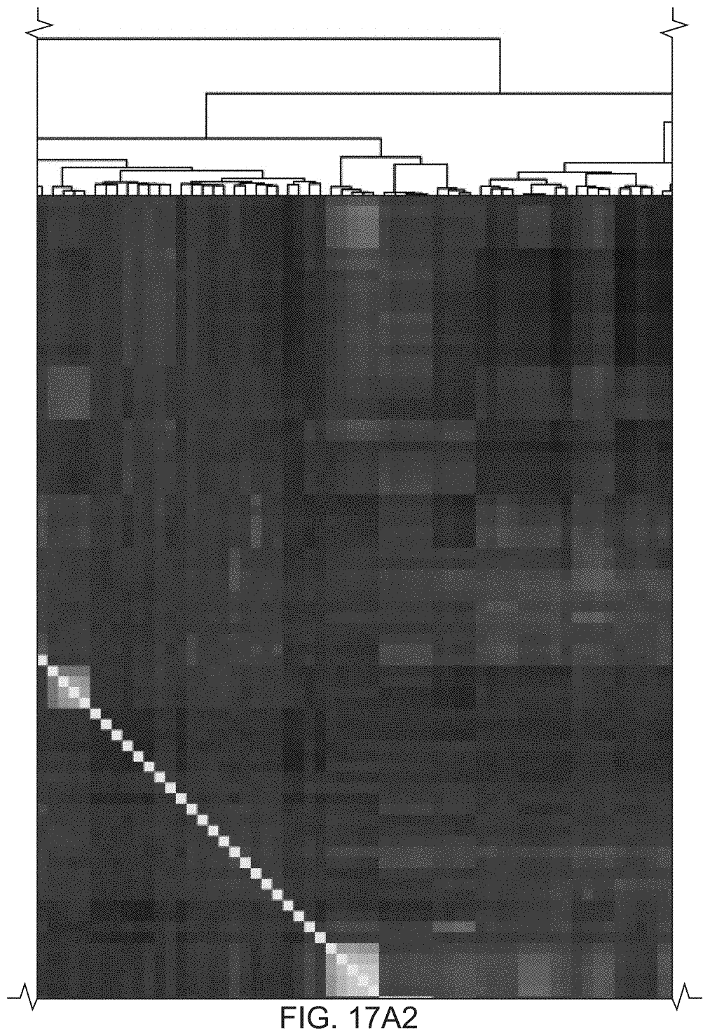

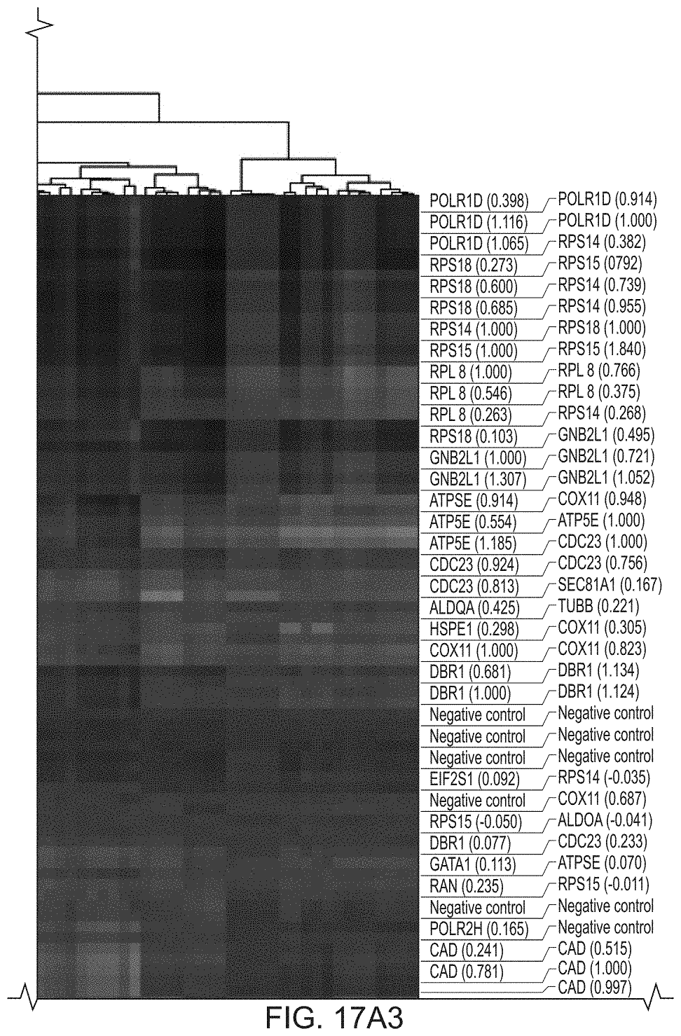

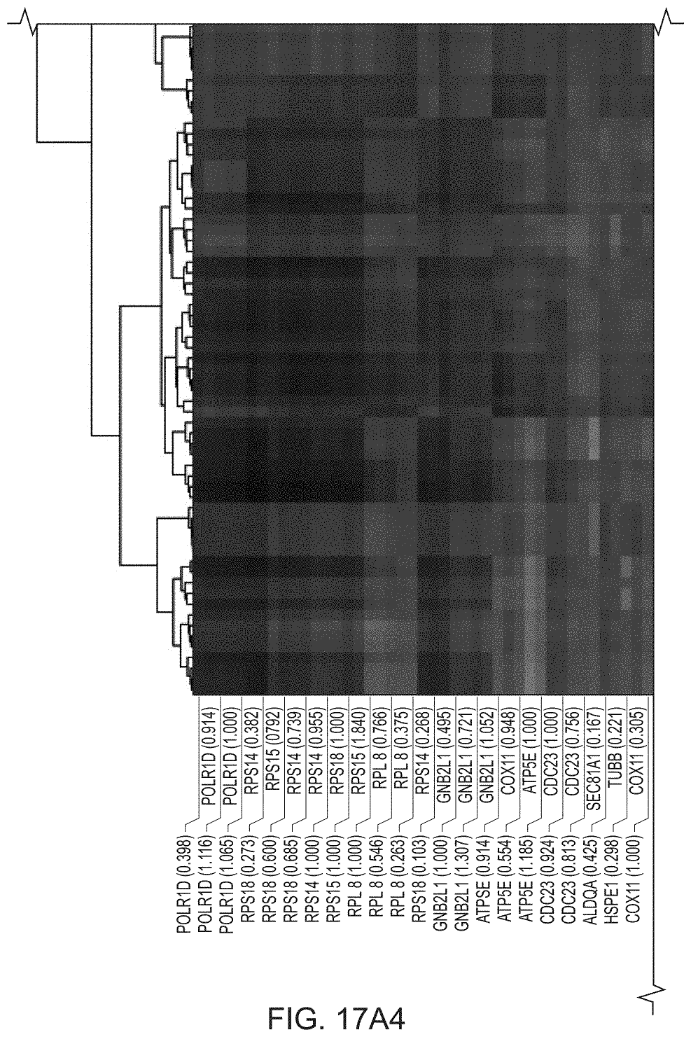

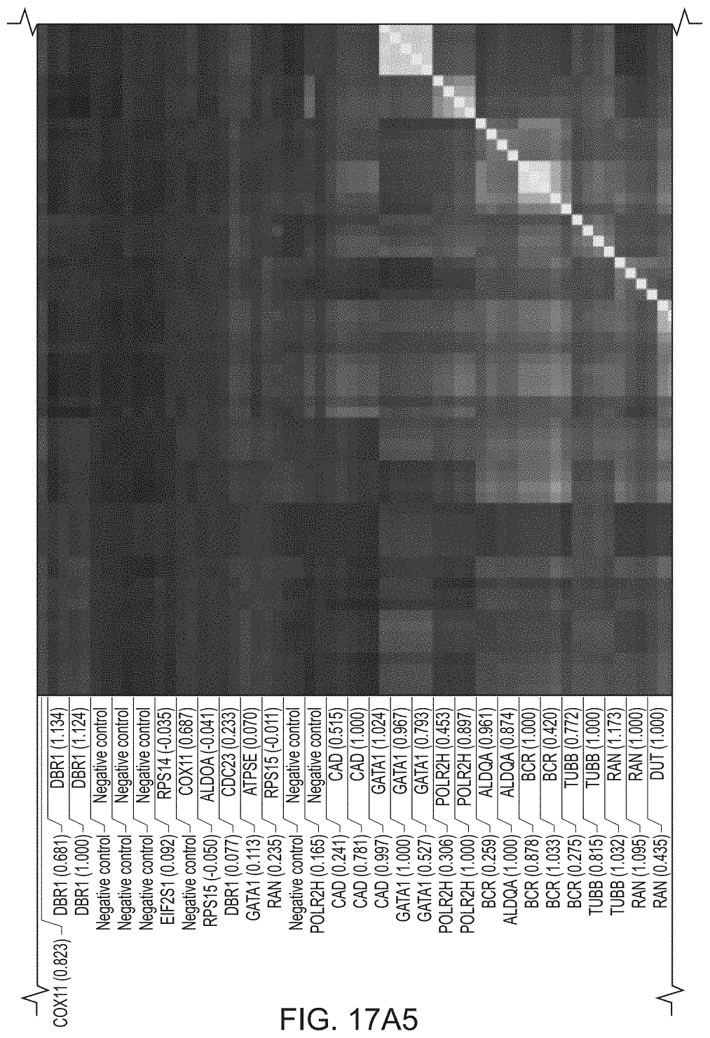

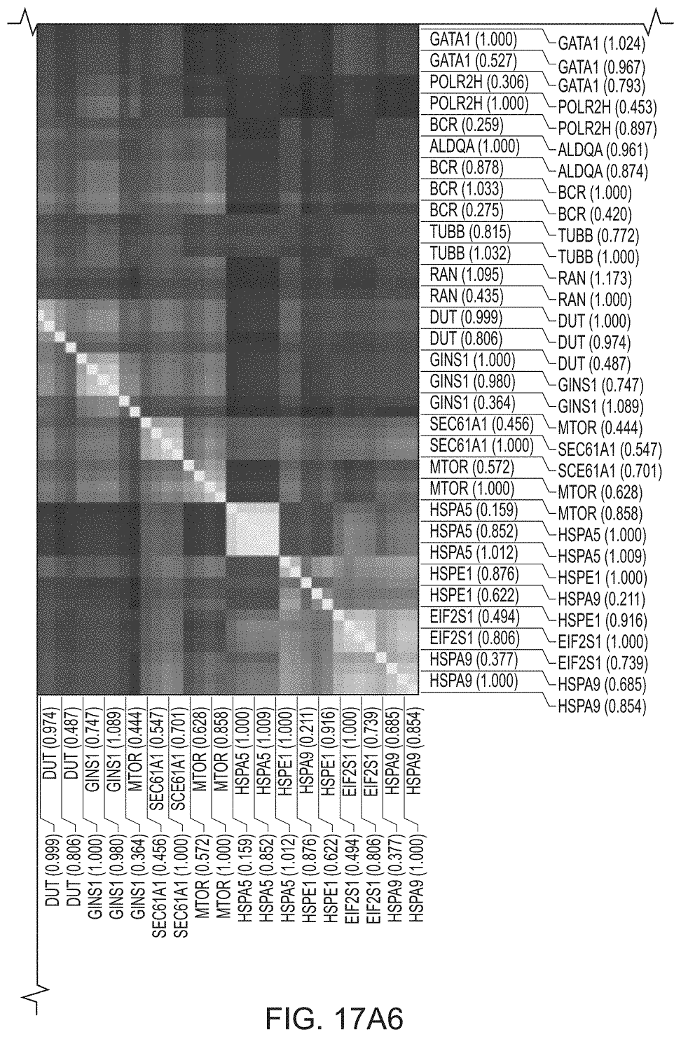

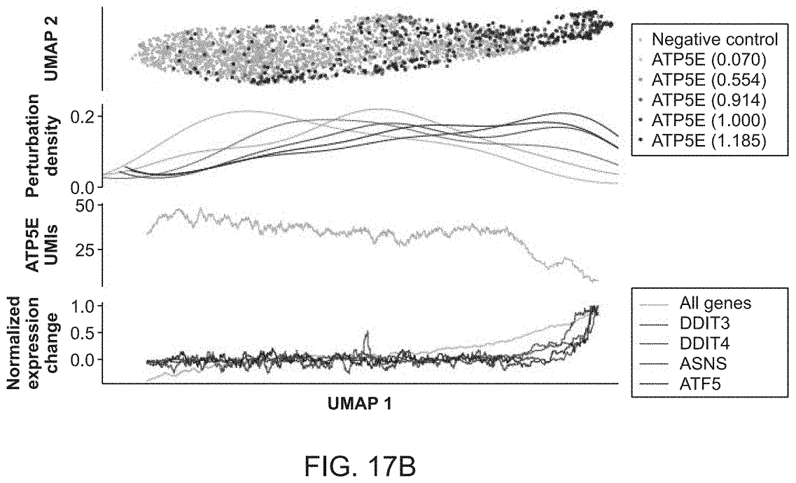

To gain further insight into the diversity of transcriptional responses induced by depletion of essential genes, we compared the transcriptional profiles of all perturbations. Clustering perturbations according to the similarity (Pearson correlation) of their bulk transcriptomes revealed multiple groups segregated by biological function, including a cluster of ribosomal proteins and POLR1D, a subunit of the rRNA-transcribing RNA polymerase I (and of RNA polymerase III), and a cluster of perturbations that activate the integrated stress response (HSPA9, HSPE1, and EIF2S1/eIF2α) (FIG. 17A). To further visualize the space of transcriptional states, we performed dimensionality reduction on the single-cell transcriptomes using UMAP (38). The resulting projection recapitulates the clustering, as indicated for example by the close proximity of cells with perturbations of HSPA9, HSPE1, and EIF2S1 (FIG. 15H). Within individual series, cells project further outward in UMAP space with increasing sgRNA activity, further highlighting that target gene expression levels are titrated on the single cell level (FIG. 15I).

Closer examination of the UMAP projection revealed more granular structure, including the grouping of a subset of cells with knockdown of ATP5E, a subunit of ATP synthase, with cells with ISR-activating perturbations (FIG. 15H). This subset of cells indeed exhibited classical features of ISR activation (FIG. 17B). The frequency of ISR activation increased with lower ATP5E mRNA levels, but even at the lowest levels some cells did not exhibit ISR activation (FIGS. 15J, 17B). These results suggest that depletion of ATP synthase under these conditions predisposes cells to activate the ISR, perhaps by exacerbating transient phases of mitochondrial stress, in a manner that is proportional to ATP synthase levels. More broadly, these results highlight the utility of titrating gene expression in probing cell biological phenotypes, especially in combination with rich phenotyping methods such as scRNA-seq.

### Discussion

Here we describe the development of allelic series of compromised sgRNAs, with each series enabling the titration of the expression of a given gene in human cells. These series, either individually or as a pool, have a broad range of applications across basic and biomedical research. We highlight the utility of the approach in extracting rich phenotypes by single-cell RNA-seq along a continuum of gene expression levels, which enabled mapping of expression levels to various phenotypes and identification of expression level-dependent cell fates.

Our approach builds on in vitro work describing the biophysical principles by which modifications to the sgRNA modulate (d)Cas9 binding on-rates and activity (13,22,39-41). In cells, modifications to the sgRNA constant region were affected by specific interactions with targeting sequences, rendering sgRNA activities difficult to predict. By contrast, the effects of mismatches on sgRNA activity followed more readily discernable biophysical principles, enabling us to apply machine learning approaches to derive the underlying rules and predict series for arbitrary sgRNAs. The resulting genome-wide in silico library enables titration of any expressed gene of interest. We also describe a compact (25,000-element) library that enables titration of 2,400 essential genes, with potential applications for example in focused screens for sensitization to chemical or genetic perturbations. Given that target gene expression levels are largely unimodally distributed in cell populations harboring sgRNA series, these sgRNAs can be combined with both single-cell or bulk population readouts. Thus, complex phenotypes as a function of gene expression levels can be recorded by a variety of techniques tailored to the particular question, such as Perturb-seq or related techniques, microscopy, bulk metabolomics or proteomics, or targeted cell biological assays, providing substantial experimental flexibility.

These sgRNA series now enable mapping expression-to-phenotype curves directly in mammalian systems, with implications for example for evolutionary biology and biomedical research. Indeed, using sgRNA series to titrate essential gene expression, we found gene-specific expression-phenotype relationships: although all genes had a threshold expression level below which cell viability dropped rapidly, the relative locations of these thresholds varied across genes, with K562 cells being particularly sensitive to depletion of GATA1 and HSPAS. This variability in threshold location suggests different buffering capacities for different genes, in line with previous findings in yeast (4), but the logic by which these buffering capacities are determined in mammalian systems remains unclear. More comprehensive efforts to generate such dose-response curves and determine the extents to which gene expression is buffered across cell models would allow for identification of patterns for different gene sets and biological processes and thereby begin to reveal the underlying principles that have shaped gene expression levels. Analogous efforts to map such dose-response curves in cancer cell types could identify specific vulnerabilities as targets for therapeutics and, vice versa, mapping these curves for cancer driver genes or genes underlying specific diseases could enable defining the corresponding therapeutic windows, i.e., the required extents of inhibition or restoration, as goals for drug development.

Our intermediate-activity sgRNAs also provide access to a diversity of cell states including loss-of-function phenotypes that otherwise may be obscured by cell death or neomorphic behavior. Thus, our approach enables positioning cells at states of interest, for example to record chemical-gene or gene-gene interactions, or near phenotypic transitions to characterize the transcriptional trajectories. These sgRNA series will also facilitate recapitulating gene expression levels of disease-relevant states such as haploinsufficiency or partial loss-of-function diseases, enabling systematic efforts to identify suppressors or modifiers as potential therapeutic targets, or modeling quantitative trait loci associated with multigenic traits in conjunction with rich phenotyping to systematically identify the mechanisms by which they interact and contribute to such traits. Finally, sgRNA allelic series can be equivalently used to titrate dCas9 occupancy and activity in other applications such as CRISPRa or dCas9-based epigenetic modifiers.

More generally, our allelic series approach now provides a tool to systematically titrate gene expression and evaluate dose-response relationships in mammalian systems. This resource should be equally enabling to systematic large-scale efforts and detailed single-gene investigations in basic cell biology, drug development, and functional genomics.

### Methods

### Reagents and Cell Lines

K562 and Jurkat cells were grown in RPMI 1640 medium (Gibco) with 25 mM HEPES, 2 mM L-glutamine, 2 g/L NaHCO₃ supplemented with 10% (v/v) standard fetal bovine serum (FBS, HyClone or VWR), 100 units/mL penicillin, 100 μg/mL streptomycin, and 2 mM L-glutamine (Gibco). HEK293T and HeLa cells were grown in Dulbecco's modified eagle medium (DMEM, Gibco) with 25 mM D-glucose, 3.7 g/L NaHCO₃, 4 mM L-glutamine and supplemented with 10% (v/v) FBS, 100 units/mL penicillin, 100 μg/mL streptomycin, and 2 mM L-glutamine. K562 and HeLa cells are derived from female patients. Jurkat cells are derived from a male patient. HEK293T are derived from a female fetus. K562 and HeLa CRISPRi cell lines were previously published (15,18). Jurkat CRISPRi cells (Clone NH7) were obtained from the Berkeley Cell Culture Facility. All cell lines were grown at 37° C. in the presence of 5% CO2. All cell lines were periodically tested for Mycoplasma contamination using the MycoAlert Plus Mycoplasma detection kit (Lonza).

### DNA Transfections and Virus Production

Lentivirus was generated by transfecting HEK239T cells with four packaging plasmids (for expression of VSV-G, Gag/Pol, Rev, and Tat, respectively) as well as the transfer plasmid using TransIT®-LT1 Transfection Reagent (Mirus Bio). Viral supernatant was harvested two days after transfection and filtered through 0.44 μm PVDF filters and/or frozen prior to transduction.

Cloning of Individual sgRNAs

Individual perfectly matched or mismatched sgRNAs were cloned essentially as described previously (15). Briefly, two complementary oligonucleotides (Integrated DNA Technologies), containing the targeting region as well as overhangs matching those left by restriction digest of the backbone with BstXI and BlpI, were annealed and ligated into an sgRNA expression vector digested with BstXI (NEB or Thermo Fisher Scientific) and BlpI (NEB) or Bpu1102I (Thermo Fisher Scientific). The ligation product was transformed into Stellar™ chemically competent E. coli cells (Takara Bio) and plasmid was prepared following standard protocols.

Individual Evaluation of sgRNA Phenotypes for GFP Knockdown

For individual evaluation of GFP knockdown phenotypes, sgRNAs were individually cloned as described above, ligated into a version of pU6-sgCXCR4-2 (marked with a puromycin resistance cassette and mCherry, Addgene #46917) (18), modified to include a BlpI site. Sequences used for individual evaluation are listed in Table 1. The sgRNA expression vectors were individually packaged into lentivirus and transduced into GFP+K562 CRISPRi cells (18) at MOI<1 (15-40% infected cells) by centrifugation at 1000×g and 33° C. for 0.5-2 h. GFP levels were recorded 10 d after transduction by flow cytometry using a FACSCelesta flow cytometer (BD Biosciences), gating for sgRNA-expressing cells (mCherry+). Experiments were performed in duplicate from the transduction step. Relative activities were defined as the fold-knockdown of each mismatched variant (GFPsgRNA[non-targeting]/GFPsgRNA[variant]) divided by the fold-knockdown of the perfectly-matched sgRNA. The background fluorescence of a GFP− strain was subtracted from all GFP values prior to other calculations. The distributions of GFP values in FIG. 1B were plotted following the example in seaborn.pydata.org/examples/kde_ridgeplot.

Design of Large-Scale Mismatched sgRNA Library

To generate the list of targeting sgRNAs for the large-scale mismatched sgRNA library, hit genes from a growth screen performed in K562 cells with the CRISPRi v2 library (19) were selected by calculating a discriminant score (phenotype z-score×−log 10(Mann-Whitney P)). Discriminant scores for negative control genes (randomly sampled groups of 10 non-targeting sgRNAs) were calculated as well, and hit genes were selected above a threshold such that 5% of the hits would be negative control genes (i.e., an estimated empirical 5% FDR). This procedure resulted in the selection of 2477 genes. Of these genes, 28 genes for which the second strongest sgRNA by absolute value had a positive growth phenotype were filtered out as these were likely to be scored as hits solely due to a single sgRNA. For the remaining 2,449 genes, the two sgRNAs with the strongest growth phenotype were selected, for a total of 4,898 perfectly matched sgRNAs.

For each of these sgRNAs, a set of 23 variant sgRNAs with mismatches was designed: 5 with a single randomly chosen mismatch within 7 bases of the PAM, 5 with a single randomly chosen mismatch 8-12 bases from the PAM, and 3 with a single randomly chosen mismatch 13-19 bases from the PAM (the first base of the targeting region was never selected for this purpose as it is an invariant G in all sgRNAs to enable transcription from the U6 promoter). The remaining 10 variants had 2 randomly chosen mismatches selected from positions −1 to −19.

To assess the off-target potential of mismatched sgRNAs, we extended our previous strategy to estimate sgRNA off-target effects (15,19). Briefly, for each target in the genome, a FASTQ entry was created for the 23 bases of the target including the PAM, with the accompanying empirical Phred score indicating an estimate of the anticipated importance of a mismatch in that base position. Bowtie (bowtie-bio.sourceforge.net) (42) was then used to align each designed sgRNA back to the genome, parameterized so that sgRNAs were considered to mutually align if and only if: a) no more than 3 mismatches existed in the PAM-proximal 12 bases and the PAM, b) the summed Phred score of all mismatched positions across the 23 bases was less than a threshold. This alignment was done iteratively with decreasing thresholds, and any sgRNAs which aligned successfully to no other site in the genome at a particular threshold were then deemed to have a specificity at said threshold. The compiled sgRNA sequences were then filtered for sgRNAs containing BstXI, BlpI, and SbfI sites, which are used during library cloning and sequencing library preparation, and 2,500 negative controls (randomly generated to match the base composition of our hCRISPRi-v2 library) were added.

Pooled Cloning of Mismatched sgRNA Libraries

Pooled sgRNA libraries were cloned largely as described previously (15,20,43). Briefly, oligonucleotide pools containing the desired elements with flanking restriction sites and PCR adapters were obtained from Agilent Technologies. The oligonucleotide pools were amplified by 15 cycles of PCR using Phusion polymerase (NEB). The PCR product was digested with BstXI (Thermo Fisher Scientific) and Bpu1102I (Thermo Fisher Scientific), purified, and ligated into BstXI/Bpu1102I-digested pCRISPRia-v2 at 16° C. for 16 h. The ligation product was purified by isopropanol precipitation and then transformed into MegaX DH10B electrocompetent cells (Thermo Fisher Scientific) by electroporation using the Gene Pulser Xcell system (Bio-Rad), transforming ˜100 ng purified ligation product per 100 μL cells. The cells were allowed to recover in 3-6 mL SOC medium for 2 h. At that point, a small 1-5 μL aliquot was removed and plated in three serial dilutions on LB plates with selective antibiotic (carbenicillin). The remainder of the culture was inoculated into 0.5 to 1 L LB supplemented with 100 μg/mL carbenicillin, grown at 37° C. with shaking at 220 rpm for 16 h and harvested by centrifugation. Colonies on the plates were counted to confirm a transformation efficiency greater than 100-fold over the number of elements (>100× coverage). The pooled sgRNA plasmid library was extracted from the cells by GigaPrep (Qiagen or Zymo Research). Even coverage of library elements was confirmed by sequencing a small aliquot on a HiSeq 4000 (Illumina).

Large-Scale Mismatched sgRNA Screen and Sequencing Library Preparation

Large-scale screens were conducted similarly to previously described screens (15,19,20). The large-scale library was transduced in duplicate into K562 CRISPRi and Jurkat CRISPRi cells at MOI<1 (percentage of transduced cells 2 days after transduction: 20-40%) by centrifugation at 1000×g and 33° C. for 2 h. Replicates were maintained separately in 0.5 L to 1 L of RPMI-1640 in 1 L spinner flasks for the course of the screen. 2 days after transduction, the cells were selected with puromycin for 2 days (K562: 2 days of 1 μg/mL; Jurkat: 1 day of 1 μg/mL and 1 day of 0.5 μg/mL), at which point transduced cells accounted for 80-95% of the population, as measured by flow cytometry using an LSR-II flow cytometer (BD Biosciences). Cells were allowed to recover for 1 day in the absence of puromycin. At this point, t0 samples with a 3000× library coverage (400×10⁶ cells) were harvested and the remaining cells were cultured further. The cells were maintained in spinner flasks by daily dilution to 0.5×10⁶ cells mL⁻¹ at an average coverage of greater than 2000 cells per sgRNA with daily measurements of cell numbers and viability on an Accuri bench-top flow cytometer (BD BioSciences) for 11 days, at which point endpoint samples were harvested by centrifugation with 3000× library coverage.

Genomic DNA was isolated from frozen cell samples and the sgRNA-encoding region was enriched, amplified, and processed for sequencing essentially as described previously (19). Briefly, genomic DNA was isolated using a NucleoSpin Blood XL kit (Macherey-Nagel), using 1 column per 100×10⁶ cells. The isolated genomic DNA was digested with 400 U SbfI-HF (NEB) per mg DNA at 37° C. for 16 h. To isolate the ˜500 bp fragment containing the sgRNA expression cassette liberated by this digest, size separation was performed using large-scale gel electrophoresis with 0.8% agarose gels. The region containing DNA between 200 and 800 bp of size was excised and DNA was purified using the NucleoSpin Gel and PCR Clean-up kit (Macherey-Nagel). The isolated DNA was quantified using a QuBit Fluorometer (Thermo Fisher Scientific) and then amplified by 23 cycles of PCR using Phusion polymerase (NEB) and appending Illumina adapter and unique sample indices in the process. Each DNA sample was divided into 5-50 individual 100 μL reactions, each with 500 ng DNA as input. To ensure base diversity during sequencing, the samples were divided into two sets, with all samples for a given replicate always being assigned to the same set. The two sets had the Illumina adapters appended in opposite orientations, such that samples in set A were sequenced from the 5′ end of the sgRNA sequence in the first 20 cycles of sequencing and samples in set B were sequenced from the 3′ end of the sgRNA sequence in the next 20 cycles of sequencing. With updates to Illumina chemistry and software, this strategy is no longer required to ensure high sequencing quality, and all samples are amplified in the same orientation. Following the PCR, all reactions for a given DNA sample were combined and a small aliquot (100-300 μL) was purified using AMPure XP beads (Beckman-Coulter) with a two-sided selection (0.65× followed by 1×). Sequencing libraries from all samples were combined and sequencing was performed on a HiSeq 4000 (Illumina) using single-read 50 runs and with two custom sequencing primers (oCRISPRi_seq_V5 and oCRISPRi_seq_V4_3′, Table 5). For samples that were amplified in the same orientation, only a single custom sequencing primer was added (oCRISPRi_seq_V5), and the samples were supplemented with a 5% PhiX spike-in.

Sequencing reads were aligned to the library sequences, counted, and quantified using the Python-based ScreenProcessing pipeline (github.com/mhorlbeck/ScreenProcessing). Calculation of phenotypes was performed as described previously (15,19,20). Untreated growth phenotypes (γ) were derived by calculating the log 2 change in enrichment of an sgRNA in the endpoint and t0 samples, subtracting the equivalent median value for all non-targeting sgRNAs, and dividing by the number of doublings of the population (15,20). To calculate relative activities, phenotypes of mismatched sgRNAs were divided by those for the corresponding perfectly matched sgRNA. Relative activities were filtered for series in which the perfectly matched sgRNA had a growth phenotype greater than 5 z-scores outside the distribution of negative control sgRNAs for all further analysis (3,147 and 2,029 sgRNA series for K562 and Jurkat cells, respectively). Relative activities from both cell lines were averaged if the series passed the z-score filter in both. All analyses were performed in Python 2.7 using a combination of Numpy (v1.14.0), Pandas (v0.23.4), and Scipy (v1.1.0).

### Design and Pooled Cloning of Constant Region Variants Library

The sequences in the library of modified constant regions were derived from the sgRNA (F+E) optimized sequence (23) modified to include a BlpI site (15). Each modified constant region was paired with 36 sgRNA targeting sequences (3 sgRNAs targeting each of 10 essential genes and six non-targeting negative control sgRNAs). The cloning strategy (described below) allowed the mutation of most positions in the sgRNA constant region. A variety of modifications were made, including substitutions of all single bases not in the BlpI restriction site (which is used for cloning), double substitutions including all substitutions at base-paired position pairs not before or in the BlpI site, and a variety of triple, quadruple, and sextuple substitutions, including base-pair-preserving substitutions at adjacent base-pairs.

The library was ordered and cloned in two parts. One part consisted of ˜100 modifications to the eight bases upstream of the BlpI restriction site. Constant region variants with mutations in this section were paired with each of the 36 targeting sequences, ordered as a pooled oligonucleotide library (Twist Biosciences), and cloned into pCRISPRia-v2 as described above. The second part consisted of ˜900 modifications to the 71 bases downstream of the BlpI restriction site. This part was cloned in two steps. First, all 36 targeting sequences were individually cloned into pCRISPRia-v2 as described above. The vectors were then pooled at an equimolar ratio and digested with BlpI (NEB) and XhoI (NEB). The modified constant region variants were ordered as a pooled oligonucleotide library (Twist Biosciences), PCR amplified with Phusion polymerase (NEB), digested with BlpI (NEB) and XhoI (NEB), and ligated into the digested vector pool, in a manner identical to previously published protocols and as described above, except for the different restriction enzymes.

Compact Mismatched sgRNA Library and Constant Region Library Screens

Screens with the compact mismatched sgRNA library and the constant region library were conducted largely as described above, with smaller modifications during the screening procedure and an updated sequencing library preparation protocol. Briefly, the libraries were transduced in duplicate into K562 CRISPRi (both libraries) or HeLa CRISPRi cells (compact mismatched sgRNA library) as described above. K562 replicates were maintained separately in 0.15 to 0.3 L of RPMI-1640 in 0.3 L spinner flasks for the course of the screen. HeLa replicates were maintained in sets of ten 15-cm plates. Cells were selected with puromycin as described above (K562: 1 day of 0.75 μg/mL and 1 day of 0.85 μg/mL; HeLa: 2 days of 0.8 μg/mL and 1 day of 1 μg/mL). The remainder of the screen was carried out at >1000× library coverage (K562 compact mismatched sgRNA library: >2000×; HeLa compact mismatched sgRNA library: >1000×; K562 constant region library: >2000×). Multiple samples were harvested after 4 to 8 days of growth. For the drug screen, 10 μM lovastatin (ApexBio) or an equivalent volume of DMSO (vehicle) was added to flasks at t=0, and 3 days later cells were pelleted and re-suspended in fresh medium. Lovastatin (12 μM) or DMSO was again added after 5 and 9 days of growth, with media exchanges 3 days after drug supplementation. Multiple samples were harvested after 4 to 8 days for the K562 and HeLa growth screens. Both drug-treated and vehicle-treated samples were harvested after 12 days for the drug screen, which allowed for a difference of 3.5 to 4.1 cell population doublings between drug- and vehicle-treated groups.

Genomic DNA was isolated from frozen cell samples as described above. The subsequent sequencing library preparation was simplified to omit the enrichment step by gel extraction. In particular, following the genomic DNA extraction, DNA was quantified by absorbance at 260 nm using a NanoDrop One spectrophotometer (Thermo Fisher Scientific) and then directly amplified by 22-23 cycles of PCR using NEBNext Ultra II Q5 PCR MasterMix (NEB), appending Illumina adapter and unique sample indices in the process. Each DNA sample was divided into 50-200 individual 100 μL reactions, each with 10 μg DNA as input. All samples were amplified using the same strategy and in the same orientation. The PCR products were purified as described above and sequencing libraries from all samples were combined. For the compact mismatched library screens, sequencing was performed on a HiSeq 4000 (Illumina) using single-read 50 runs with a 5% PhiX spike-in and a custom sequencing primer (oCRISPRi_seq_V5, Table 5). For the constant region screens, the PCR primers were adapted to allow for amplification of the entire constant region and to append a standard Illumina read 2 primer binding site (Table 5). Sequencing was then performed in the same manner including the custom sequencing primer (oCRISPRi_seq_v5) and a 5% PhiX spike-in, but using paired-read 150 runs.

Sequencing reads were processed as described above. Sequences and rankings for individual sgRNAs are available in Table 6 for the constant region screen.

### Generation and Evaluation of Individual Constant Region Variants by RT-qPCR

Constant region variants were evaluated in the background of a constant region with an additional base pair substitution in the first stem loop (fourth base pair changed from AT to GC25). Ten constant region variants with average relative activities between 0.2 and 0.8 from the screen and carrying substitutions after the BlpI site were selected (Table 5). Cloning of individual constant regions was performed essentially as the cloning of sgRNA targeting regions, described above, except that the BlpI and XhoI restriction sites were used for cloning (the XhoI site is immediately downstream of the constant region) and that cloning was performed with a variant of pCRISPRia-v2 (marked with a puromycin resistance cassette and BFP, Addgene #84832)19. For each of the ten constant region variants as well as the constant region carrying only the stem loop substitution, two different targeting regions against DPH2 were then cloned as described above (Table 1). These 22 vectors as well as a vector with a non-targeting negative control sgRNA (Table 1) were individually packaged into lentivirus and transduced into K562 CRISPRi cells at MOI<1 (10-50% infected cells) by centrifugation at 1000×g and 33° C. for 2 h. Cells were allowed to recover for 2 days and then selected to purity with puromycin (1.5-3 μg/mL), as assessed by measuring the fraction of BFP-positive cells by flow cytometry on an LSR-II (BD Biosciences), allowed to recover for 1 day, and harvested in aliquots of 0.5-2×10⁶ cells for RNA extraction. RNA was extracted using the RNeasy Mini kit (Qiagen) with on-column DNase digestion (Qiagen) and reverse-transcribed using SuperScript II Reverse Transcriptase (Thermo Fisher Scientific) with oligo(dT) primers in the presence of RNaseOUT Recombinant Ribonuclease Inhibitor (Thermo Fisher Scientific). Quantitative PCR (qPCR) reactions were performed in 22 μL reactions by adding 20 μL master mix containing 1.1× Colorless GoTaq Reaction Buffer (Promega), 0.7 mM MgCl2, dNTPs (0.2 mM each), primers (0.75 μM each), and 0.1×SYBR Green with GoTaq DNA polymerase (Promega) to 2 μL cDNA or water. Reactions were run on a LightCycler 480 Instrument (Roche). For each cDNA sample, reactions were set up with qPCR primers against DPH2 and ACTB (sequences listed in Table 5). Experiments were performed in technical triplicates.

### Machine Learning

In order to establish a subset of highly active sgRNAs with which to train a machine learning model, we filtered for perfectly matched sgRNAs with a growth phenotype greater than 10 z-scores outside the distribution of negative control sgRNAs in the K562 and/or Jurkat pooled screens (K562 γ<−0.21; Jurkat γ<−0.35). All singly mismatched variants derived from sgRNAs passing the filter were then included, and relative activities were calculated as described previously, averaging the replicate measurements for each sgRNA. In cases where a perfectly matched sgRNA passed the filter in the K562 and Jurkat screen, the average relative activity across both cell types was calculated for each mismatched variant; otherwise the relative activities for only one cell type were considered. This filtering scheme resulted in 26,248 mismatched sgRNAs comprising 2,034 series targeting 1,292 genes, with approximately 40% of relative activity values averaged from K562 and Jurkat cells.

For each sgRNA, a set of features was defined based on the sequences of the genomic target and the mismatched sgRNA. First, the genomic sequence extending from 22 bases 5′ of the beginning of the PAM to 1 base 3′ of the end of the PAM (26 bases in all) is binarized into a 2D array of shape (4, 26), with 0s and 1s indicating the absence or presence of a particular nucleotide at each position, respectively. Next, a similar array is constructed representing the mismatch imparted by the sgRNA, with an additional potential mismatch at the 5′ terminus of the sgRNA (position −20), which invariably begins with G in our libraries due to the mU6 promoter. Thus, the mismatched sequence array is identical to the genomic sequence array except for 1 or 2 positions. Finally, the arrays are stacked into a 3D volume of shape (4, 26, 2), which serves as the feature set for a particular sgRNA.

The training set of sgRNAs was established by randomly selecting 80% of sgRNA series, with the remaining 20% set aside for model validation. A convolutional neural network (CNN) regression model was then designed using Keras (keras.io/) with a TensorFlow backend engine, consisting of two sequential convolution layers, a max pooling layer, a flattening layer, and finally a three-layer fully connected network terminating in a single neuron. Additional regularization was achieved by adding dropout layers after the pooling step and between each fully connected layer. To penalize the model for ignoring under-represented sgRNA classes (e.g., those with intermediate relative activity), training sgRNAs were binned according to relative activity, and sample weights inversely proportional to the population in each bin were assigned. Hyperparameters were optimized using a randomized grid search with 3-fold cross-validation with the training set as input. Parameters included the size, shape, stride, and number of convolution filters, the pooling strategy, the number of neurons and layers in the dense network, the extent of dropout applied at each regularization step, the activation functions in each layer, the loss function, and the model optimizer. Ultimately, 20 CNN models with identical starting parameters were individually trained for 8 epochs in batches of 32 sgRNAs. Performance was assessed by computing the average prediction of the 20-model ensemble for each validation sgRNA and comparing it to the measured value.

A linear regression model was trained on the same set of sgRNAs, albeit with modified features more suited for this approach. These features include the identities of bases in and around the PAM, whether the invariant G at the 5′ end of the sgRNA is base paired, the GC content of the sgRNA, the change in GC content due to the point mutation, the location of the protospacer relative to the annotated transcription start site, the identities of the 3 RNA bases on either side of the mismatch, and the location and type of each mismatch. All features were binarized except for GC and delta GC content. In total, each sgRNA was represented by a vector of 270 features, 228 of which describe the mismatch position and type (19 possible positions by 12 possible types). Prior to training, feature vectors were z-normalized to set the mean to 0 and variance to 1. Finally, an elastic net linear regression model was created using the scikit-learn Python package (scikit-learn.org), and key hyperparameters (alpha and L1 ratio) were optimized using a grid search with 3-fold cross validation during training.

### Design of Compact Library

Genes targeted by the compact allelic series library were required to have at least one perfectly matched sgRNA with a growth phenotype greater than 2 z-scores outside the distribution of negative control sgRNAs (γ<−0.04) in a single replicate of a K562 pooled screen (this work or Horlbeck et al. (19)). By this metric, 4,722 unique sgRNAs targeting 2,405 essential genes were included. Next, for each perfectly matched sgRNA, variants containing all 57 single mismatches in the targeting sequence (positions −19 to −1) were generated in silico, and sequences with off-target binding potential in the human genome were filtered out as described for the large-scale library. Remaining variant sgRNAs were whitelisted for potential selection in subsequent steps.

For each gene being targeted, if both of the perfectly matched sgRNAs imparted growth phenotypes greater than 3 z-scores outside the distribution of negative controls (γ<−0.06) in this work's large-scale K562 screen, then one series of 4 variant sgRNAs was generated from each. Otherwise, one series of 8 variants was generated from the sgRNA with the stronger phenotype. Both perfectly matched sgRNAs were included regardless of their growth phenotype, for a total of 2 perfectly matched and 8 mismatched sgRNAs per gene.

In order to select mismatched sgRNAs, we first divided the relative activity space into 6 bins with edges at 0.1, 0.3, 0.5, 0.7, and 0.9. For each series, we attempted to select sgRNAs from each of the middle 4 bins (centers at 0.2, 0.4, 0.6, and 0.8 relative activity) as measured in this work's K562 screen. If multiple sgRNAs were available in a particular bin, they were prioritized based on distance to the center of the bin and variance between replicate measurements. If no previously measured sgRNA was available in a given bin, then the CNN model was run on all whitelisted (novel) mismatched sgRNAs belonging to that series, and sgRNAs were selected based on predicted activity as needed. In total, the compact library was composed of 4,722 unique perfectly matched sgRNAs, 19,210 unique mismatched sgRNAs, and 1,202 non-targeting control sgRNAs. Approximately 68% of mismatched sgRNAs were evaluated in previous screens (72% single mismatches, 28% double mismatches), with the remaining 32% imputed from the CNN model (all single mismatches).

### Perturb-Seq

The Perturb-seq experiment targeted 25 genes involved in a diverse range of essential functions (Table 2). For each target gene, the original sgRNAs and 4-5 mismatched sgRNAs covering the range from full relative activity to low relative activity were chosen from the large-scale screen. These 128 targeting sgRNAs as well as 10 non-targeting negative control sgRNAs (Table 1) were individually cloned into a modified variant of the CROP-seq vector (33,34) as described above, except into the different vector. Lentivirus was individually packaged for each of the 138 sgRNAs and was harvested and frozen in array. To determine viral titers, each virus was individually transduced into K562 CRISPRi cells by centrifugation at 1000×g and 33° C. for 2 h, and the fraction of transduced cells was quantified as BFP+ cells using an LSR-II flow cytometer (BD Biosciences) 48 h after transduction.

To generate transduced cells for single-cell RNA-seq analysis, virus for all 138 sgRNAs was pooled immediately before transduction and then transduced into K562 CRISPRi cells by centrifugation at 1000×g and 33° C. for 2 h. To achieve even representation at the intended time of single-cell analysis, the virus pooling was adjusted both for titer and expected growth-rate defects. 3 d after transduction, transduced (BFP+) cells were selected using FACS on a FACSAria2 (BD Biosciences) and then resuspended in conditioned media (RPMI formulated as described above except supplemented with 20% FBS and 20% supernatant of an exponentially growing K562 culture). 2 d after sorting, the cells were loaded onto three lanes of a Chromium Single Cell 3′ V2 chip (10× Genomics) at 1000 cells/μL and processed according to the manufacturer's instructions.

The CROP-seq sgRNA barcode was PCR amplified from the final single cell RNA-seq libraries with a primer specific to the sgRNA expression cassette (oBA503, Table 5) and a standard P5 primer (Table 5), purified on a Blue Pippin 1.5% agarose cassette (Sage Science) with size selection range 436-534 bp, and pooled with the single cell RNA-seq libraries at a ratio of 1:100. The libraries were sequenced on a HiSeq 4000 according to the manufacturer's instructions (10× Genomics).

To measure the growth rate defects conferred by each sgRNA for comparison with the transcriptional phenotypes, samples of 500,000 transduced cells were taken from the same transduced cell population used in the Perturb-seq experiment on days two, seven, and twelve after transduction. Genomic DNA was extracted using the Nucleospin Blood kit (Macherey-Nagel) and sgRNA amplicons were prepared as described previously and above (19), albeit with no genomic DNA digestion or gel purification, and sequenced on HiSeq 4000 as described above for the other screens. Growth phenotypes were calculated by comparing normalized sgRNA abundances at day seven and twelve to those at day two, as described above. Read counts and growth phenotypes (γ and relative activity) for individual sgRNAs are available in Table 3 and Table 4, respectively. Relative sgRNA activities measured at day seven (five days of growth) were used to assign sgRNA activities in further analysis.

### Perturb-Seq Data Analysis

Raw and processed Perturb-seq data are available at GEO under accession code GSE132080.

### Cell Barcode and UMI Calling, Assignment of Perturbations

UMI count tables with UMI counts for all genes in each individual cell were calculated from the raw sequencing data using CellRanger 2.1.1 (10× Genomics) with default settings. Perturbation calling was performed as described previously (27). Briefly, reads from the specifically amplified sgRNA barcode libraries were aligned to a list of expected sgRNA barcode sequences using bowtie (flags: -v3 -q -ml). Reads with common UMI and barcode identity were then collapsed to counts for each cell barcode, producing a list of possible perturbation identities contained by that cell. A proposed perturbation identity was identified as “confident” if it met thresholds derived by examining the distributions of reads and UMIs across all cells and candidate identities: (1) reads >50, (2) UMIs>3, and (3) coverage (reads/UMI) in the upper mode of the observed distribution across all candidate identities. As described previously (44), perturbation identities were called for any cell barcode with greater than 2,000 UMIs to enable capture of cells with strong growth defects. Any cell barcode containing two or more confident identities was deemed a “multiplet”, and may arise from either multiple infection or simultaneous encapsulation of more than one cell in a droplet during single-cell RNA sequencing. Cell barcodes passing the 2,000 UMI threshold and bearing a single, unambiguous perturbation barcode were included in all subsequent analyses.

### Expression Normalization

Some portions of analysis use normalized expression data. We used a relative normalization procedure based on comparison to the gene expression observed in control cells bearing non-targeting sgRNAs, as described previously (27).

Total UMI counts for each cell barcode are normalized to have the median number of UMIs observed in control cells.

For each gene x, expression across all cell barcodes is z-normalized with respect to the mean (μ_x) and standard deviation (σ_x) observed in control cells:

x_“normalized”=(x−μ_x)/σ_x

Following this normalization, control cells have average expression 0 (and standard deviation 1) for all genes. Negative/positive values therefore represent under/overexpression relative to control.

### Target Gene Quantification

Expression levels of genes targeted by a given sgRNA were quantified by normalizing UMI counts of the targeted gene to the total UMI count for each individual cell (FIG. 13). Considering raw UMI counts of the targeted gene (FIG. 14) or z-normalized target gene expression as described above yielded similar results. Note that the sgRNA targeting BCR is toxic due to knockdown of the BCR-ABL1 fusion present in K562 cells. Knockdown was apparent both in BCR and ABL1 expression, but we used BCR expression for further analysis as there are likely additional copies of ABL1 that are not fused to BCR (and thus would not be affected by the BCR-targeting sgRNA) contributing to ABL1 expression.

### Cell Cycle Analysis

Calling of cell cycle stages was performed using a similar approach to Macosko et al. (45) and largely as described in Adamson and Norman et al. (27). Briefly, lists of marker genes showing specific expression in different cell cycle stages from the literature were first adapted to K562 cells by restricting to those that showed highly correlated expression within our experiment. The total (log 2-normalized) expression of each set of marker genes was used to create scores for each cell cycle stage within each cell, and these scores were then z-normalized across all cells. Each cell was assigned to the cell cycle stage with the highest score.

### Differential Gene Expression Analysis

We took two approaches to differential expression, as described previously (44). For both approaches, we only considered genes with expression greater than 0.25 UMIs per cell on average across all cells. First, for a given gene, we could assess the changes in the expression distribution of that gene induced by a given genetic perturbation by comparing to the expression distribution observed in control cells bearing non-targeting sgRNAs. We performed this comparison using a two-sample Kolmogorov-Smirnov test and corrected for multiple hypothesis testing at an FDR of 0.001 using the Benjamini-Yekutieli procedure.

We also exploited a machine learning approach that potentially allows correlated expression patterns to be detected and that scales beyond two sample comparisons. Perturbed cells and control cells bearing non-targeting sgRNAs were each used as training data for a random forest classifier that was trained to predict which sgRNA a cell contained from its transcriptional state. As part of the training process the classifier ranks which genes have the most prognostic power in predicting sgRNA identity, which by construction will tend to vary across condition. For most further analysis, the top 100-300 genes by prognostic power were then considered.

### Constructing Mean Expression Profiles

For some analyses, expression profiles were averaged across all cells with the same perturbation. In general, this was done simply by calculating the mean z-normalized expression of all genes with mean expression level of 0.25 UMI or higher across all cells in the experiment or within the specific considered subpopulation (usually all cells with sgRNAs targeting a given gene as well as all control cells with non-targeting sgRNAs).

### UMAP Dimensionality Reduction

For UMAP dimensionality reduction 38 of all cells, the 300 genes with the highest prognostic power in distinguishing cells by targeted gene as ranked by a random forest classifier were selected. Dimensionality reduction was then performed on the z-normalized single-cell expression profiles of these 300 genes using the following parameters: n_neighbors=40, min_dist=0.1, metric=‘euclidean’, spread=1.0. UMAP dimensionality reduction of subpopulations containing only cells with perturbation of a given gene or control cells was performed analogously but using the expression profiles of the 100 genes with the highest prognostic power and using n_neighbors=15.

From the UMAP projection, we concluded that ˜5% cells had misassigned sgRNA identities, as evident for example by the presence of cells with negative control sgRNAs within the cluster of cells with HSPAS knockdown. These cells had confidently assigned single perturbations and only expressed the corresponding barcode transcript, suggesting that they did not evade our doublet detection algorithm. We speculate that these cells expressed two different sgRNAs but silenced expression of one of the reporter transcripts. Given the strong trends in the results above, we concluded that this rate of misassignment did not substantially affect our ability to identify trends within cell populations.

### ISR Scores

Magnitude of ISR activation in individual cells was quantified as activation of the PERK (EIF2AK3) regulon from the gene set and activation coefficients determined previously (27).

### REFERENCES

1. Huang, N., Lee, I., Marcotte, E. M. & Hurles, M. E. Characterising and Predicting Haploinsufficiency in the Human Genome. PLOS Genet. 6, e1001154 (2010).
2. Rest, J. S. et al. Nonlinear Fitness Consequences of Variation in Expression Level of a Eukaryotic Gene. Mol. Biol. Evol. 30, 448-456 (2013).
3. Bauer, C. R., Li, S. & Siegal, M. L. Essential gene disruptions reveal complex relationships between phenotypic robustness, pleiotropy, and fitness. Mol. Syst. Biol. 11, 773-773 (2015).
4. Keren, L. et al. Massively Parallel Interrogation of the Effects of Gene Expression Levels on Fitness. Cell 166, 1282-1294.e18 (2016).
5. Dykhuizen, D. E., Dean, A. M. & Hard, D. L. Metabolic Flux and Fitness. Genetics 115, 25-31 (1987).
6. Dekel, E. & Alon, U. Optimality and evolutionary tuning of the expression level of a protein. Nature 436, 588-592 (2005).
7. Alper, H., Fischer, C., Nevoigt, E. & Stephanopoulos, G. Tuning genetic control through promoter engineering. Proc. Natl. Acad. Sci. 102, 12678-12683 (2005).
8. Perfeito, L., Ghozzi, S., Berg, J., Schnetz, K. & Lassig, M. Nonlinear Fitness Landscape of a Molecular Pathway. PLOS Genet. 7, e1002160 (2011).
9. Michaels, Y. S. et al. Precise tuning of gene expression levels in mammalian cells. Nat. Commun. 10, 818 (2019).
10. Moore, R., Chandrahas, A. & Bleris, L. Transcription Activator-like Effectors: A Toolkit for Synthetic Biology. ACS Synth. Biol. 3, 708-716 (2014).
11. Dominguez, A. A., Lim, W. A. & Qi, L. S. Beyond editing: repurposing CRISPR-Cas9 for precision genome regulation and interrogation. Nat. Rev. Mol. Cell Biol. 17, 5-15 (2016).
12. Jinek, M. et al. A Programmable Dual-RNA-Guided DNA Endonuclease in Adaptive Bacterial Immunity. Science 337, 816-821 (2012).
13. Sternberg, S. H., Redding, S., Jinek, M., Greene, E. C. & Doudna, J. A. DNA interrogation by the CRISPR RNA-guided endonuclease Cas9. Nature 507, 62-67 (2014).
14. Szczelkun, M. D. et al. Direct observation of R-loop formation by single RNA-guided Cas9 and Cascade effector complexes. Proc. Natl. Acad. Sci. 111, 9798-9803 (2014).
15. Gilbert, L. A. et al. Genome-Scale CRISPR-Mediated Control of Gene Repression and Activation. Cell 159, 647-661 (2014).
16. Nishimasu, H. et al. Crystal Structure of Cas9 in Complex with Guide RNA and Target DNA. Cell 156, 935-949 (2014).
17. Kocak, D. D. et al. Increasing the specificity of CRISPR systems with engineered RNA secondary structures. Nat. Biotechnol. 37, 657 (2019).
18. Gilbert, L. A. et al. CRISPR-Mediated Modular RNA-Guided Regulation of Transcription in Eukaryotes. Cell 154, 442-451 (2013).
19. Horlbeck, M. A. et al. Compact and highly active next-generation libraries for CRISPR-mediated gene repression and activation. eLife 5, e19760 (2016).
20. Kampmann, M., Bassik, M. C. & Weissman, J. S. Integrated platform for genome-wide screening and construction of high-density genetic interaction maps in mammalian cells. Proc. Natl. Acad. Sci. 110, E2317-E2326 (2013).
21. Doench, J. G. et al. Optimized sgRNA design to maximize activity and minimize off-target effects of CRISPR-Cas9. Nat. Biotechnol. 34, 184-191 (2016).
22. Boyle, E. A. et al. High-throughput biochemical profiling reveals sequence determinants of dCas9 off-target binding and unbinding. Proc. Natl. Acad. Sci. 114, 5461-5466 (2017).
23. Chen, B. et al. Dynamic Imaging of Genomic Loci in Living Human Cells by an Optimized CRISPR/Cas System. Cell 155, 1479-1491 (2013).
24. Dang, Y. et al. Optimizing sgRNA structure to improve CRISPR-Cas9 knockout efficiency. Genome Biol. 16, 280 (2015).
25. Grevet, J. D. et al. Domain-focused CRISPR screen identifies HRI as a fetal hemoglobin regulator in human erythroid cells. Science 361, 285-290 (2018).
26. Briner, A. E. et al. Guide RNA Functional Modules Direct Cas9 Activity and Orthogonality. Mol. Cell 56, 333-339 (2014).
27. Adamson, B. et al. A Multiplexed Single-Cell CRISPR Screening Platform Enables Systematic Dissection of the Unfolded Protein Response. Cell 167, 1867-1882.e21 (2016).
28. Eraslan, G., Avsec, 2., Gagneur, J. & Theis, F. J. Deep learning: new computational modelling techniques for genomics. Nat. Rev. Genet. 1 (2019). doi:10.1038/s41576-019-0122-6
29. Kim, H. K. et al. Deep learning improves prediction of CRISPR-Cpf1 guide RNA activity. Nat. Biotechnol. 36, 239-241 (2018).
30. Luo, J., Chen, W., Xue, L. & Tang, B. Prediction of activity and specificity of CRISPR-Cpf1 using convolutional deep learning neural networks. BMC Bioinformatics 20, 332 (2019).
31. Dixit, A. et al. Perturb-Seq: Dissecting Molecular Circuits with Scalable Single-Cell RNA Profiling of Pooled Genetic Screens. Cell 167, 1853-1866.e17 (2016).
32. Jaitin, D. A. et al. Dissecting Immune Circuits by Linking CRISPR-Pooled Screens with Single-Cell RNA-Seq. Cell 167, 1883-1896.e15 (2016).
33. Datlinger, P. et al. Pooled CRISPR screening with single-cell transcriptome readout. Nat. Methods 14, 297-301 (2017).
34. Replogle, J. M. et al. Direct capture of CRISPR guides enables scalable, multiplexed, and multi-omic Perturb-seq. bioRxiv 503367 (2018). doi:10.1101/503367
35. Grosveld, G. et al. The chronic myelocytic cell line K562 contains a breakpoint in bcr and produces a chimeric bcr/c-abl transcript. Mol. Cell. Biol. 6, 607-616 (1986).
36. Shtivelman, E., Lifshitz, B., Gale, R. P. & Canaani, E. Fused transcript of abl and bcr genes in chronic myelogenous leukaemia. Nature 315, 550 (1985).
37. Harding, H. P. et al. An Integrated Stress Response Regulates Amino Acid Metabolism and Resistance to Oxidative Stress. Mol. Cell 11, 619-633 (2003).
38. McInnes, L., Healy, J. & Melville, J. UMAP: Uniform Manifold Approximation and Projection for Dimension Reduction. ArXiv180203426 Cs Stat (2018).
39. Semenova, E. et al. Interference by clustered regularly interspaced short palindromic repeat (CRISPR) RNA is governed by a seed sequence. Proc. Natl. Acad. Sci. 108, 10098-10103 (2011).
40. Wiedenheft, B. et al. RNA-guided complex from a bacterial immune system enhances target recognition through seed sequence interactions. Proc. Natl. Acad. Sci. 108, 10092-10097 (2011).
41. Hsu, P. D. et al. DNA targeting specificity of RNA-guided Cas9 nucleases. Nat. Biotechnol. 31, 827-832 (2013).
42. Langmead, B., Trapnell, C., Pop, M. & Salzberg, S. L. Ultrafast and memory-efficient alignment of short DNA sequences to the human genome. Genome Biol. 10, R25 (2009).
43. Bassik, M. C. et al. Rapid creation and quantitative monitoring of high coverage shRNA libraries. Nat. Methods 6, 443-445 (2009).
44. Norman, T. M. et al. Exploring genetic interaction manifolds constructed from rich phenotypes. bioRxiv 601096 (2019). doi:10.1101/601096
45. Macosko, E. Z. et al. Highly Parallel Genome-wide Expression Profiling of Individual Cells Using Nanoliter Droplets. Cell 161, 1202-1214 (2015).

TABLE 1
sgRNA sequences used in this study.

| | | | SEQ | |
|---|---|---|---|---|
| | | | ID | Target_ |
| Experiment | Name | Sequence | NO: | gene |
| GFP single | EGFP-NT2 | GACCAGGATGGGCACCACCC | 1 | EGFP |
| mismatches | | | | |
| constant region RT- | DPH2_ + _44435896.24-all | GAGTAAGCAGTCCTGGCACCC | 2 | DPH2 |
| qPCR | | | | |
| constant region RT- | DPH2_ − _44435877.23-all | GATGTTTAGCAGCCCTGCCG | 3 | DPH2 |
| qPCR | | | | |
| constant region RT- | non-targeting_00564 | GCCGATGGTCTTGTACTACA | 4 | neg_ctrl |
| qPCR | | | | |
| constant region | RPL9_ + _39460483.23-P1P2 | GGATGTTTCTGTGCTCGTGG | 5 | RPL9 |
| screen | | | | |
| constant region | RPL9_ + _39460504.23-P1P2 | GCTGCGTCTACTGCGAGGTA | 6 | RPL9 |
| screen | | | | |
| constant region | RPL9_ + _39460476.23-P1P2 | GCTGTGCTCGTGGGGGTACT | 7 | RPL9 |
| screen | | | | |
| constant region | HSPE1_ − _198365117.23-P1P2 | GCGGACTGCGAGTCTCTTTG | 8 | HSPE1 |
| screen | | | | |
| constant region | HSPE1_ + _198365089.23-P1P2 | GGAGACTCGCAGTCCGGCCC | 9 | HSPE1 |
| screen | | | | |
| constant region | HSPE1_ − _198365304.23-P1P2 | GGCCCGATGGCACCTTGGAG | 10 | HSPE1 |
| screen | | | | |
| constant region | POLR1D_ + _28196016.23-P1 | GGGAAGCAAGGACCGACCGA | 11 | POLR1D |
| screen | | | | |
| constant region | POLR1D_ + _28196036.23-P1 | GCGAGGCGCGGAGGCGAAGC | 12 | POLR1D |
| screen | | | | |
| constant region | POLR1D_ + _28196012.23-P1 | GGCAAGGACCGACCGACGGA | 13 | POLR1D |
| screen | | | | |
| constant region | SNRPD2_ + _46195119.23-P1P2 | GAGGCCGGGCTAGGCTTAGG | 14 | SNRPD2 |
| screen | | | | |
| constant region | SNRPD2_ + _46195138.23-P1P2 | GGCGTAGTGACCATCATGTG | 15 | SNRPD2 |
| screen | | | | |
| constant region | SNRPD2_ − _46195150.23-P1P2 | GCTAGCCCGGCCTCACATGA | 16 | SNRPD2 |
| screen | | | | |
| constant region | CDC23_ + _137548970.23-P1P2 | GAGTACCTCCATGGTCCCGG | 17 | CDC23 |
| screen | | | | |
| constant region | CDC23_ − _137548987.23-P1P2 | GACAGCCACCGGGACCATGG | 18 | CDC23 |
| screen | | | | |
| constant region | CDC23_ − _137548622.23-P1P2 | GCCAGTGACAGGGCACTCAG | 19 | CDC23 |
| screen | | | | |
| constant region | CAD_ + _27440280.23-P1P2 | GGCTGGAGAGAAGCCGGGCG | 20 | CAD |
| screen | | | | |
| constant region | CAD_ + _27440373.23-P1P2 | GCGAGTACGGAGAAGCGGGA | 21 | CAD |
| screen | | | | |
| constant region | CAD_ + _27440253.23-P1P2 | GTAGGAGCCTCGGGCGCGCT | 22 | CAD |
| screen | | | | |
| constant region | TUBB_ + _30688126.23-P1 | GCGGCAGGAAGGTTCTGAGA | 23 | TUBB |
| screen | | | | |
| constant region | TUBB_ + _30688173.23-P1 | GAGGTTGGAATGCGCCCCAG | 24 | TUBB |
| screen | | | | |
| constant region | TUBB_ + _30688145.23-P1 | GCAGCGAGGTGCAAACGCGA | 25 | TUBB |
| screen | | | | |
| constant region | POLR2H_ − _184081237.23-P1P2 | GGTGCACGTACTCCCAACTG | 26 | POLR2H |
| screen | | | | |
| constant region | POLR2H_ + _184081227.23-P1P2 | GTGAGAGCGCGACCACAGTT | 27 | POLR2H |
| screen | | | | |
| constant region | POLR2H_ + _184081251.23-P1P2 | GGGGCCACGAGAGCAGCAGA | 28 | POLR2H |
| screen | | | | |
| constant region | DUT_ + _48624414.23-P1P2 | GAGGCGAGCGAGGAGACCAC | 29 | DUT |
| screen | | | | |
| constant region | DUT_ − _48624041.23-P1P2 | GCGTCTGGAAGGAATCCACG | 30 | DUT |
| screen | | | | |
| constant region | DUT_ − _48623651.23-P1P2 | GCAGGACGGGCGCGTCTTCA | 31 | DUT |
| screen | | | | |
| constant region | DNAJC19_ + _180707414.23- | GGGATGAGCCGTGCTCCCGG | 32 | DNAJC19 |
| screen | P1P2 | | | |
| constant region | DNAJC19_ + _180707118.23- | GCTTGCCTGGAACTCCTGTA | 33 | DNAJC19 |
| screen | P1P2 | | | |
| constant region | DNAJC19_ + _180707491.23- | GGGCGCCTGTGCTTGAGGTT | 34 | DNAJC19 |
| screen | P1P2 | | | |
| constant region | non-targeting_03786 | GTGGCCGTTCATGGGACCGG | 35 | neg_ctrl |
| screen | | | | |
| constant region | non-targeting_03636 | GACAATATCTGGATCGCCAA | 36 | neg_ctrl |
| screen | | | | |
| constant region | non-targeting_03478 | GGATGGGCTCGCCTGGCCAG | 37 | neg_ctrl |
| screen | | | | |
| constant region | non-targeting_03229 | GGTCCCACGGCGAAGCGACT | 38 | neg_ctrl |
| screen | | | | |
| constant region | non-targeting_00564 | GCCGATGGTCTTGTACTACA | 4 | neg_ctrl |
| screen | | | | |
| constant region | non-targeting_00763 | GGCGCGGGCCCCATAAAAAC | 39 | neg_ctrl |
| screen | | | | |
| perturb-seq | RPS18_ + _33239917.23-P1P2_00 | GCTGCGATGCCGCTGGATCA | 40 | RPS18 |
| perturb-seq | RPS18_ + _33239917.23-P1P2_01 | GCTGCAATGCCGCTGGATCA | 41 | RPS18 |
| perturb-seq | RPS18_ + _33239917.23-P1P2_02 | GCTGGGATGCCGCTGGATCA | 42 | RPS18 |
| perturb-seq | RPS18_ + _33239917.23-P1P2_08 | GCTGCGATTCCGCTGGATCA | 43 | RPS18 |
| perturb-seq | RPS18_ + _33239917.23-P1P2_04 | GCTGCGATCCCGCTGGATCA | 44 | RPS18 |
| perturb-seq | RPS14_ + _149829238.23- | GAGGCCCGGGCGCGACAATC | 45 | RPS14 |
| | P1P2_00 | | | |
| perturb-seq | RPS14_ + _149829238.23- | GAGACCCGGGCGCGACAATC | 46 | RPS14 |
| | P1P2_01 | | | |
| perturb-seq | RPS14_ + _149829238.23- | GAGGCCCTGGCGCGACAATC | 47 | RPS14 |
| | P1P2_02 | | | |
| perturb-seq | RPS14_ + _149829238.23- | GAGGCCCGCGCGCGACAATC | 48 | RPS14 |
| | P1P2_04 | | | |
| perturb-seq | RPS14_ + _149829238.23- | GAGGCCCGGGCGCGACAGTC | 49 | RPS14 |
| | P1P2_13 | | | |
| perturb-seq | RPS14_ + _149829238.23- | GAGGCCCGGGCTCGACAATC | 50 | RPS14 |
| | P1P2_08 | | | |
| perturb-seq | RPL9_ + _39460483.23-P1P2_00 | GGATGTTTCTGTGCTCGTGG | 5 | RPL9 |
| perturb-seq | RPL9_ + _39460483.23-P1P2_01 | GGATGATTCTGTGCTCGTGG | 51 | RPL9 |
| perturb-seq | RPL9_ + _39460483.23-P1P2_05 | GGATGTTTCGGTGCTCGTGG | 52 | RPL9 |
| perturb-seq | RPL9_ + _39460483.23-P1P2_04 | GGATGTTTCAGTGCTCGTGG | 53 | RPL9 |
| perturb-seq | RPL9_ + _39460483.23-P1P2_07 | GGATGTTTCTGCGCTCGTGG | 54 | RPL9 |
| perturb-seq | GNB2L1_ + _180670873.23- | GTGCAAGGCGGCGGCAGGAG | 55 | GNB2L1 |
| | P1P2_00 | | | |
| perturb-seq | GNB2L1_ + _180670873.23- | GTGCAAGGTGGCGGCAGGAG | 56 | GNB2L1 |
| | P1P2_08 | | | |
| perturb-seq | GNB2L1_ + _180670873.23- | GTGCAAGGCGGCGGCGGGAG | 57 | GNB2L1 |
| | P1P2_13 | | | |
| perturb-seq | GNB2L1_ + _180670873.23- | GTGCAAGGCGGGGGCAGGAG | 58 | GNB2L1 |
| | P1P2_07 | | | |
| perturb-seq | GNB2L1_ + _180670873.23- | GTGCAAGACGGCGGCAGGAG | 59 | GNB2L1 |
| | P1P2_02 | | | |
| perturb-seq | RPS15_ − _1438413.23-P1P2_00 | GACCAAAGCGATCTCTTCTG | 60 | RPS15 |
| | | C63 | | |
| perturb-seq | RPS15_ − _1438413.23-P1P2_07 | GACCAAAGCGGTCTCTTCTG | 61 | RPS15 |
| perturb-seq | RPS15_ − _1438413.23-P1P2_02 | GACCAAGGCGATCTCTTCTG | 62 | RPS15 |
| perturb-seq | RPS15_ − _1438413.23-P1P2_12 | GACCAAAGCGATCTCTTGTG | 63 | RPS15 |
| perturb-seq | RPS15_ − _1438413.23-P1P2_01 | GACCAAACCGATCTCTTCTG | 64 | RPS15 |
| perturb-seq | HSPE1_ + _198365089.23- | GGAGACTCGCAGTCCGGCCC | 9 | HSPE1 |
| | P1P2_00 | | | |
| perturb-seq | HSPE1_ + _198365089.23- | GGAGACACGCAGTCCGGCCC | 65 | HSPE1 |
| | P1P2_01 | | | |
| perturb-seq | HSPE1_ + _198365089.23- | GGTGACTCGCAGTCCGGCCC | 66 | HSPE1 |
| | P1P2_03 | | | |
| perturb-seq | HSPE1_ + _198365089.23- | GGAGACTGGCAGTCCGGCCC | 67 | HSPE1 |
| | P1P2_02 | | | |
| perturb-seq | HSPE1_ + _198365089.23- | GGAGACTCGCAGTCCTGCCC | 68 | HSPE1 |
| | P1P2_14 | | | |
| perturb-seq | RAN_ + _131356438.23-P1P2_00 | GGCGGTCGCTGCGCTTAGGG | 69 | RAN |
| perturb-seq | RAN_ + _131356438.23-P1P2_02 | GGCGGCCGCTGCGCTTAGGG | 70 | RAN |
| perturb-seq | RAN_ + _131356438.23-P1P2_03 | GGGGGTCGCTGCGCTTAGGG | 71 | RAN |
| perturb-seq | RAN_ + _131356438.23-P1P2_04 | GGCGGTCGCGGCGCTTAGGG | 72 | RAN |
| perturb-seq | RAN_ + _131356438.23-P1P2_12 | GGCGGTCGCTGCGCTTAGGT | 73 | RAN |
| perturb-seq | POLR1D_ + _28196016.23-P1_00 | GGGAAGCAAGGACCGACCGA | 11 | POLR1D |
| perturb-seq | POLR1D_ + _28196016.23-P1_08 | GGGAAGCAGGGACCGACCGA | 74 | POLR1D |
| perturb-seq | POLR1D_ + _28196016.23-P1_03 | GGTAAGCAAGGACCGACCGA | 75 | POLR1D |
| perturb-seq | POLR1D_ + _28196016.23-P1_01 | GGGAAGCCAGGACCGACCGA | 76 | POLR1D |
| perturb-seq | POLR1D_ + _28196016.23-P1_07 | GGGAAGCAAGGAGCGACCGA | 77 | POLR1D |
| perturb-seq | DBR1_ + _137893744.23- | GTTTGCAGGAGTCTACACCC | 78 | DBR1 |
| | P1P2_00 | | | |
| perturb-seq | DBR1_ + _137893744.23- | GATTGCAGGAGTCTACACCC | 79 | DBR1 |
| | P1P2_01 | | | |
| perturb-seq | DBR1_ + _137893744.23- | GTTTGCAGGGGTCTACACCC | 80 | DBR1 |
| | P1P2_07 | | | |
| perturb-seq | DBR1_ + _137893744.23- | GTTTGCAGGAGTGTACACCC | 81 | DBR1 |
| | P1P2_05 | | | |
| perturb-seq | DBR1_ + _137893744.23- | GTTTGCAGTAGTCTACACCC | 82 | DBR1 |
| | P1P2_08 | | | |
| perturb-seq | SEC61A1_ − _127771295.23- | GGCACTGACGTGTCTCTCGG | 83 | SEC61A1 |
| | P1_00 | | | |
| perturb-seq | SEC61A1_ − _127771295.23- | GGCGCTGACGTGTCTCTCGG | 84 | SEC61A1 |
| | P1_02 | | | |
| perturb-seq | SEC61A1_ − _127771295.23- | GGCACTGTCGTGTCTCTCGG | 85 | SEC61A1 |
| | P1_01 | | | |
| perturb-seq | SEC61A1_ − _127771295.23- | GGTACTGACGTGTCTCTCGG | 86 | SEC61A1 |
| | P1_03 | | | |
| perturb-seq | SEC61A1_ − _127771295.23- | GGCACTGAAGTGTCTCTCGG | 87 | SEC61A1 |
| | P1_04 | | | |
| perturb-seq | HSPA5_ + _128003624.23- | GAGCCGAGTAGGCGACGGTG | 88 | HSPA5 |
| | P1P2_00 | | | |
| perturb-seq | HSPA5_ + _128003624.23- | GAGCCGAGAAGGCGACGGTG | 89 | HSPA5 |
| | P1P2_04 | | | |
| perturb-seq | HSPA5_ + _128003624.23- | GAGCCGAGTGGGCGACGGTG | 90 | HSPA5 |
| | P1P2_08 | | | |
| perturb-seq | HSPA5_ + _128003624.23- | GAACCGAGTAGGCGACGGTG | 91 | HSPA5 |
| | P1P2_01 | | | |
| perturb-seq | HSPA5_ + _128003624.23- | GAGCCGAGTAGACGACGGTG | 92 | HSPA5 |
| | P1P2_06 | | | |
| perturb-seq | GINS1_ − _25388381.23-P1P2_00 | GGACTAGAACGAAAGGAGTG | 93 | GINS1 |
| perturb-seq | GINS1_ − _25388381.23-P1P2_08 | GGACTAGAGCGAAAGGAGTG | 94 | GINS1 |
| perturb-seq | GINS1_ − _25388381.23-P1P2_06 | GGACTAGAACGGAAGGAGTG | 95 | GINS1 |
| perturb-seq | GINS1_ − _25388381.23-P1P2_03 | GGACTATAACGAAAGGAGTG | 96 | GINS1 |
| perturb-seq | GINS1_ − _25388381.23-P1P2_14 | GGACTAGAACGAAAGGAGCG | 97 | GINS1 |
| perturb-seq | CDC23_ − _137548987.23- | GACAGCCACCGGGACCATGG | 18 | CDC23 |
| | P1P2_00 | | | |
| perturb-seq | CDC23_ − _137548987.23- | GACAGCTACCGGGACCATGG | 98 | CDC23 |
| | P1P2_02 | | | |
| perturb-seq | CDC23_ − _137548987.23- | GACAGCCATCGGGACCATGG | 99 | CDC23 |
| | P1P2_08 | | | |
| perturb-seq | CDC23_ − _137548987.23- | GACAGCCAACGGGACCATGG | 100 | CDC23 |
| | P1P2_04 | | | |
| perturb-seq | CDC23_ − _137548987.23- | GACAGCCACCGGGACCACGG | 101 | CDC23 |
| | P1P2_11 | | | |
| perturb-seq | CAD_ + _27440280.23-P1P2_00 | GGCTGGAGAGAAGCCGGGCG | 20 | CAD |
| perturb-seq | CAD_ + _27440280.23-P1P2_03 | GGCTGGTGAGAAGCCGGGCG | 102 | CAD |
| perturb-seq | CAD_ + _27440280.23-P1P2_07 | GGCTGGAGCGAAGCCGGGCG | 103 | CAD |
| perturb-seq | CAD_ + _27440280.23-P1P2_06 | GGCTGGAGAGTAGCCGGGCG | 104 | CAD |
| perturb-seq | CAD_ + _27440280.23-P1P2_13 | GGCTGGAGAGAAGCCTGGCG | 105 | CAD |
| perturb-seq | TUBB_ + _30688126.23-P1_00 | GCGGCAGGAAGGTTCTGAGA | 23 | TUBB |
| perturb-seq | TUBB_ + _30688126.23-P1_01 | GCAGCAGGAAGGTTCTGAGA | 106 | TUBB |
| perturb-seq | TUBB_ + _30688126.23-P1_06 | GCGGCAGGACGGTTCTGAGA | 107 | TUBB |
| perturb-seq | TUBB_ + _30688126.23-P1_03 | GCGGCAGCAAGGTTCTGAGA | 108 | TUBB |
| perturb-seq | TUBB_ + _30688126.23-P1_10 | GCGGCAGGAAGGTTCAGAGA | 109 | TUBB |
| perturb-seq | DUT_ + _48624411.23-P1P2_00 | GCGAGCGAGGAGACCACCGG | 110 | DUT |
| perturb-seq | DUT_ + _48624411.23-P1P2_01 | GCCAGCGAGGAGACCACCGG | 111 | DUT |
| perturb-seq | DUT_ + _48624411.23-P1P2_08 | GCGAGCGAGGAGGCCACCGG | 112 | DUT |
| perturb-seq | DUT_ + _48624411.23-P1P2_07 | GCGAGCGAGGAGCCCACCGG | 113 | DUT |
| perturb-seq | DUT_ + _48624411.23-P1P2_10 | GCGAGCGAGGAGACCAACGG | 114 | DUT |
| perturb-seq | POLR2H_ + _184081251.23- | GGGGCCACGAGAGCAGCAGA | 28 | POLR2H |
| | P1P2_00 | | | |
| perturb-seq | POLR2H_ + _184081251.23- | GGGGCCACGAGAGCAGCGGA | 115 | POLR2H |
| | P1P2_11 | | | |
| perturb-seq | POLR2H_ + _184081251.23- | GGGGCCACGCGAGCAGCAGA | 116 | POLR2H |
| | P1P2_08 | | | |
| perturb-seq | POLR2H_ + _184081251.23- | GGGGCCACGAGAGCAGGAGA | 117 | POLR2H |
| | P1P2_12 | | | |
| perturb-seq | POLR2H_ + _184081251.23- | GGGGCCACGAGTGCAGCAGA | 118 | POLR2H |
| | P1P2_07 | | | |
| perturb-seq | GATA1_ − _48645022.23- | GTGAGCTTGCCACATCCCCA | 119 | GATA1 |
| | P1P2_00 | | | |
| perturb-seq | GATA1_ − _48645022.23- | GTGCGCTTGCCACATCCCCA | 120 | GATA1 |
| | P1P2_03 | | | |
| perturb-seq | GATA1_ − _48645022.23- | GTGAGCTTACCACATCCCCA | 121 | GATA1 |
| | P1P2_04 | | | |
| perturb-seq | GATA1_ − _48645022.23- | GTGAGCTTTCCACATCCCCA | 122 | GATA1 |
| | P1P2_08 | | | |
| perturb-seq | GATA1_ − _48645022.23- | GTGAGCTTGCGACATCCCCA | 123 | GATA1 |
| | P1P2_06 | | | |
| perturb-seq | GATA1_ − _48645022.23- | GTGAGCTTGCCACATCCGCA | 124 | GATA1 |
| | P1P2_12 | | | |
| perturb-seq | BCR_ + _23523092.23-P1P2_00 | GCGCGCGGGGCCCGTCTCAG | 125 | BCR |
| perturb-seq | BCR_ + _23523092.23-P1P2_07 | GCGCGCGGGGCTCGTCTCAG | 126 | BCR |
| perturb-seq | BCR_ + _23523092.23-P1P2_04 | GCGCGCGGAGCCCGTCTCAG | 127 | BCR |
| perturb-seq | BCR_ + _23523092.23-P1P2_05 | GCGCGCGGCGCCCGTCTCAG | 128 | BCR |
| perturb-seq | BCR_ + _23523092.23-P1P2_15 | GCGCGCGGGGCCCGTCGCAG | 129 | BCR |
| perturb-seq | BCR_ + _23523092.23-P1P2_13 | GCGCGCGGGGCCCATCTCAG | 130 | BCR |
| perturb-seq | HSPA9_ − _137911079.23- | GGAGCTGCGCGATGCGGTGG | 131 | HSPA9 |
| | P1P2_00 | | | |
| perturb-seq | HSPA9_ − _137911079.23- | GGAGCTGCGGGATGCGGTGG | 132 | HSPA9 |
| | P1P2_07 | | | |
| perturb-seq | HSPA9_ − _137911079.23- | GGAGTTGCGCGATGCGGTGG | 133 | HSPA9 |
| | P1P2_02 | | | |
| perturb-seq | HSPA9_ − _137911079.23- | GGAGCTGCTCGATGCGGTGG | 134 | HSPA9 |
| | P1P2_08 | | | |
| perturb-seq | HSPA9_ − _137911079.23- | GGAGCTGCGCAATGCGGTGG | 135 | HSPA9 |
| | P1P2_04 | | | |
| perturb-seq | EIF2S1_ − _67827080.23-P1P2_00 | GAGCGAAGCGCACGCTGAGG | 136 | EIF2S1 |
| perturb-seq | EIF2S1_ − _67827080.23-P1P2_06 | GAGCGAAGCGCGCGCTGAGG | 137 | EIF2S1 |
| perturb-seq | EIF2S1_ − _67827080.23-P1P2_02 | GAGCGCAGCGCACGCTGAGG | 138 | EIF2S1 |
| perturb-seq | EIF2S1_ − _67827080.23-P1P2_01 | GAGCGAAACGCACGCTGAGG | 139 | EIF2S1 |
| perturb-seq | EIF2S1_ − _67827080.23-P1P2_07 | GAGCGAAGCGCTCGCTGAGG | 140 | EIF2S1 |
| perturb-seq | COX11_ + _53045977.23- | GGCTCTGGCGTCCTGGATGG | 141 | COX11 |
| | P1P2_00 | | | |
| perturb-seq | COX11- + _53045977.23- | GGCTCTGTCGTCCTGGATGG | 142 | COX11 |
| | P1P2_03 | | | |
| perturb-seq | COX11_ + _53045977.23- | GGCTCTGGCGCCCTGGATGG | 143 | COX11 |
| | P1P2_04 | | | |
| perturb-seq | COX11_ + _53045977.23- | GGCTCTGGCGTCTTGGATGG | 144 | COX11 |
| | P1P2_05 | | | |
| perturb-seq | COX11_ + _53045977.23- | GGCTCTGGCGTCCCGGATGG | 145 | COX11 |
| | P1P2_10 | | | |
| perturb-seq | MTOR_ + _11322547.23-P1P2_00 | GGGCAGGGGGCCTGAAGCGG | 146 | MTOR |
| perturb-seq | MTOR_+ _11322547.23-P1P2_07 | GGGCAGGGGGTCTGAAGCGG | 147 | MTOR |
| perturb-seq | MTOR_ + _11322547.23-P1P2_05 | GGGCAGGGGGCTTGAAGCGG | 148 | MTOR |
| perturb-seq | MTOR_ + _11322547.23-P1P2_06 | GGGCAGGGGGGCTGAAGCGG | 149 | MTOR |
| perturb-seq | MTOR_ + _11322547.23-P1P2_10 | GGGCAGGGGGCCTGAAGCAG | 150 | MTOR |
| perturb-seq | ATP5E_− _57607036.23-P1P2_00 | GGTGTCCAGGGGCACTCTGT | 151 | ATP5E |
| perturb-seq | ATP5E_ − _57607036.23-P1P2_01 | GGTGTCCTGGGGCACTCTGT | 152 | ATP5E |
| perturb-seq | ATP5E_ − _57607036.23-P1P2_16 | GGTGTCCAGGGGCGCTCTGT | 153 | ATP5E |
| perturb-seq | ATP5E_ − _57607036.23-P1P2_04 | GGTGTCCAGGAGCACTCTGT | 154 | ATP5E |
| perturb-seq | ATP5E_ − _57607036.23-P1P2_14 | GGTGTCCAGGGGCACTGTGT | 155 | ATP5E |
| perturb-seq | ALDOA_ + _30077139.23- | GGTCACCAGGACCCCTTCTG | 156 | ALDOA |
| | P1P2_00 | | | |
| perturb-seq | ALDOA_ + _30077139.23- | GGTCACCAGGATCCCTTCTG | 157 | ALDOA |
| | P1P2_06 | | | |
| perturb-seq | ALDOA_ + _30077139.23- | GGTCACCAGGCCCCCTTCTG | 158 | ALDOA |
| | P1P2_07 | | | |
| perturb-seq | ALDOA_ + _30077139.23- | GGTCACCAGGACCGCTTCTG | 159 | ALDOA |
| | P1P2_14 | | | |
| perturb-seq | ALDOA_ + _30077139.23- | GGTCACCAGGACCCCTTTTG | 160 | ALDOA |
| | P1P2_13 | | | |
| perturb-seq | non-targeting_00001 | GTGCACCCGGCTAGGACCGG | 161 | neg_ctrl |
| perturb-seq | non-targeting_00028 | GGTGGCCTTTGCAATTGGCG | 162 | neg_ctrl |
| perturb-seq | non-targeting_00054 | GGGCCTGGACGAGCCTAAAA | 163 | neg_ctrl |
| perturb-seq | non-targeting_00089 | GGGGTGAGGGTCCAATTCGG | 164 | neg_ctrl |
| perturb-seq | non-targeting_00217 | GTGAACTCAAAAATCCCGAC | 165 | neg_ctrl |
| perturb-seq | non-targeting_00283 | GGGCCGACGGATAGGAGGGA | 166 | neg_ctrl |
| perturb-seq | non-targeting_00406 | GGCGCCGGACTGGACCTCGA | 167 | neg_ctrl |
| perturb-seq | non-targeting_00527 | GTGGGAGCAGATCAAGACTC | 168 | neg_ctrl |
| perturb-seq | non-targeting_00802 | GCACGACGCTCCGGCACGCG | 169 | neg_ctrl |
| perturb-seq | non-targeting_01040 | GTACGGCATGGCGCACTGCG | 170 | neg_ctrl |

TABLE 2
Perturb-seq gene descriptions.

| | |
|---|---|
| ALDOA | Aldolase A; glycolytic enzyme |
| ATP5E | ATP synthase subunit |
| BCR-ABL | Fusion gene; drives CML-derived K562 cells |
| CAD | Pyrimidine nucleotide biosynthesis enzyme; catalyzes |
| | multiple pathway steps |
| CDC23 | Anaphase promoting complex/cyclosome component |
| COX11 | Mitochondrial respiratory chain; cytochrome c oxidase |
| | assembly factor |
| DBR1 | Lariat debranching enzyme; required for lariat intron |
| | degradation after splicing |
| DUT | dUTP pyrophosphatase; involved in thymidine biosynthesis |
| EIF2S1 | eIF2α; Translation initiation factor; translational control factor |
| GATA1 | Erythroid-lineage transcription factor |
| GINS1 | DNA replication initiation factor |
| GNB2L1 | RACK1; 40s ribosomal protein; associated with numerous |
| | signalling processes |
| HSPA5 | BiP; ER chaperone involved in protein import and folding |
| HSPA9 | Mortalin; Mitochondrial chaperone and import factor |
| HSPE1 | Mitochondrial chaperone |
| MTOR | Kinase; regulates growth, metabolism, and autophagy |
| POLR1D | RNA polymerase I and III subunit |
| POLR2H | RNA polymerase I, II, and III subunit |
| RAN | G-protein that controls protein and RNA transport through the |
| | nuclear pore |
| RPL9 | Ribosomal protein L9 |
| RPS14 | Ribosomal protein S14 |
| RPS15 | Ribosomal protein S15 |
| RPS18 | Ribosomal protein S18 |
| SEC61A1 | ER translocon component |
| TUBB | beta-tubulin; structural component of microtubules |

TABLE 3
Perturb-seq pooled growth sgRNA counts.

| | T0 | d10 | d5 |
|---|---|---|---|
| ALDOA_+_30077139.23-P1P2_00 | 5280 | 2781 | 4056 |
| ALDOA_+_30077139.23-P1P2_06 | 6015 | 3500 | 4831 |
| ALDOA_+_30077139.23-P1P2_07 | 4830 | 3028 | 4284 |
| ALDOA_+_30077139.23-P1P2_13 | 6699 | 26890 | 16944 |
| ALDOA_+_30077139.23-P1P2_14 | 3603 | 6076 | 5347 |
| ATP5E_−_57607036.23-P1P2_00 | 8197 | 9475 | 12109 |
| ATP5E_−_57607036.23-P1P2_01 | 7774 | 8806 | 10487 |
| ATP5E_−_57607036.23-P1P2_04 | 7209 | 14860 | 13256 |
| ATP5E_−_57607036.23-P1P2_14 | 4611 | 15257 | 10750 |
| ATP5E_−_57607036.23-P1P2_16 | 6210 | 9964 | 9571 |
| BCR_+_23523092.23-P1P2_00 | 9644 | 2333 | 2250 |
| BCR_+_23523092.23-P1P2_04 | 5355 | 2119 | 1660 |
| BCR_+_23523092.23-P1P2_05 | 13439 | 15537 | 12165 |
| BCR_+_23523092.23-P1P2_07 | 8081 | 2183 | 1744 |
| BCR_+_23523092.23-P1P2_13 | 4304 | 7063 | 5668 |
| BCR_+_23523092.23-P1P2_15 | 5377 | 8085 | 6829 |
| CAD_+_27440280.23-P1P2_00 | 8671 | 785 | 2464 |
| CAD_+_27440280.23-P1P2_03 | 7290 | 907 | 2087 |
| CAD_+_27440280.23-P1P2_06 | 6199 | 4365 | 4967 |
| CAD_+_27440280.23-P1P2_07 | 13241 | 4019 | 6008 |
| CAD_+_27440280.23-P1P2_13 | 11874 | 19130 | 17097 |
| CDC23_−_137548987.23-P1P2_00 | 8182 | 854 | 757 |
| CDC23_−_137548987.23-P1P2_02 | 7014 | 1192 | 832 |
| CDC23_−_137548987.23-P1P2_04 | 8019 | 1646 | 1646 |
| CDC23_−_137548987.23-P1P2_08 | 8986 | 1710 | 1531 |
| CDC23_−_137548987.23-P1P2_11 | 12707 | 16682 | 14320 |
| COX11_+_53045977.23-P1P2_00 | 8084 | 6198 | 11785 |
| COX11_+_53045977.23-P1P2_03 | 11251 | 9184 | 16852 |
| COX11_+_53045977.23-P1P2_04 | 5234 | 5047 | 8343 |
| COX11_+_53045977.23-P1P2_05 | 5205 | 11496 | 10766 |
| COX11_+_53045977.23-P1P2_10 | 5206 | 11271 | 8887 |
| DBR1_+_137893744.23-P1P2_00 | 13446 | 3583 | 9171 |
| DBR1_+_137893744.23-P1P2_01 | 9446 | 1824 | 5512 |
| DBR1_+_137893744.23-P1P2_05 | 6569 | 4748 | 6705 |
| DBR1_+_137893744.23-P1P2_07 | 8500 | 2550 | 4894 |
| DBR1_+_137893744.23-P1P2_08 | 5326 | 15989 | 11651 |
| DUT_+_48624411.23-P1P2_00 | 14025 | 1570 | 3755 |
| DUT_+_48624411.23-P1P2_01 | 25227 | 3576 | 6764 |
| DUT_+_48624411.23-P1P2_07 | 4601 | 1157 | 1509 |
| DUT_+_48624411.23-P1P2_08 | 15356 | 2392 | 4351 |
| DUT_+_48624411.23-P1P2_10 | 6538 | 4466 | 5403 |
| EIF2S1_−_67827080.23-P1P2_00 | 5718 | 1318 | 1123 |
| EIF2S1_−_67827080.23-P1P2_01 | 5433 | 4065 | 3799 |
| EIF2S1_−_67827080.23-P1P2_02 | 8035 | 2582 | 2570 |
| EIF2S1_−_67827080.23-P1P2_06 | 4549 | 2436 | 1718 |
| EIF2S1_−_67827080.23-P1P2_07 | 6931 | 22309 | 13281 |
| GATA1_−_48645022.23-P1P2_00 | 5712 | 757 | 955 |
| GATA1_−_48645022.23-P1P2_03 | 12276 | 1534 | 1927 |
| GATA1_−_48645022.23-P1P2_04 | 4714 | 668 | 860 |
| GATA1_−_48645022.23-P1P2_06 | 9440 | 5489 | 5580 |
| GATA1_−_48645022.23-P1P2_08 | 7028 | 1873 | 2043 |
| GATA1_−_48645022.23-P1P2_12 | 4081 | 11548 | 7289 |
| GINS1_−_25388381.23-P1P2_00 | 3621 | 280 | 547 |
| GINS1_−_25388381.23-P1P2_03 | 9799 | 2755 | 2982 |
| GINS1_−_25388381.23-P1P2_06 | 11452 | 1219 | 1828 |
| GINS1_−_25388381.23-P1P2_08 | 18173 | 1756 | 2461 |
| GINS1_−_25388381.23-P1P2_14 | 6443 | 6093 | 5833 |
| GNB2L1_+_180670873.23-P1P2_00 | 2280 | 1685 | 2456 |
| GNB2L1_+_180670873.23-P1P2_02 | 3839 | 7618 | 6216 |
| GNB2L1_+_180670873.23-P1P2_07 | 9894 | 8738 | 8322 |
| GNB2L1_+_180670873.23-P1P2_08 | 24451 | 17083 | 25247 |
| GNB2L1_+_180670873.23-P1P2_13 | 4708 | 5991 | 6350 |
| HSPA5_+_128003624.23-P1P2_00 | 5785 | 2176 | 1756 |
| HSPA5_+_128003624.23-P1P2_01 | 7580 | 3812 | 3124 |
| HSPA5_+_128003624.23-P1P2_04 | 11091 | 4282 | 3304 |
| HSPA5_+_128003624.23-P1P2_06 | 10180 | 23714 | 17649 |
| HSPA5_+_128003624.23-P1P2_08 | 10148 | 3487 | 3005 |
| HSPA9_−_137911079.23-P1P2_00 | 5450 | 835 | 944 |
| HSPA9_−_137911079.23-P1P2_02 | 4345 | 1872 | 1727 |
| HSPA9_−_137911079.23-P1P2_04 | 6754 | 10829 | 9346 |
| HSPA9_−_137911079.23-P1P2_07 | 5941 | 1463 | 1513 |
| HSPA9_−_137911079.23-P1P2_08 | 3137 | 2726 | 2803 |
| HSPE1_+_198365089.23-P1P2_00 | 6813 | 1179 | 2348 |
| HSPE1_+_198365089.23-P1P2_01 | 9669 | 2663 | 4228 |
| HSPE1_+_198365089.23-P1P2_02 | 7969 | 4437 | 5731 |
| HSPE1_+_198365089.23-P1P2_03 | 7473 | 2279 | 3034 |
| HSPE1_+_198365089.23-P1P2_14 | 4808 | 6498 | 6501 |
| MTOR_+_11322547.23-P1P2_00 | 17632 | 3144 | 6328 |
| MTOR_+_11322547.23-P1P2_05 | 5595 | 3324 | 4083 |
| MTOR_+_11322547.23-P1P2_06 | 4142 | 3174 | 3358 |
| MTOR_+_11322547.23-P1P2_07 | 6761 | 1899 | 3183 |
| MTOR_+_11322547.23-P1P2_10 | 7076 | 7827 | 7332 |
| POLR1D_+_28196016.23-P1_00 | 11671 | 1496 | 3429 |
| POLR1D_+_28196016.23-P1_01 | 12679 | 2528 | 4460 |
| POLR1D_+_28196016.23-P1_03 | 10266 | 933 | 2365 |
| POLR1D_+_28196016.23-P1_07 | 15589 | 16285 | 16283 |
| POLR1D_+_28196016.23-P1_08 | 16414 | 1986 | 4205 |
| POLR2H_+_184081251.23-P1P2_00 | 9498 | 1103 | 947 |
| POLR2H_+_184081251.23-P1P2_07 | 4472 | 8153 | 6381 |
| POLR2H_+_184081251.23-P1P2_08 | 6134 | 3869 | 3492 |
| POLR2H_+_184081251.23-P1P2_11 | 5900 | 1144 | 898 |
| POLR2H_+_184081251.23-P1P2_12 | 5334 | 5996 | 4854 |
| RAN_+_131356438.23-P1P2_00 | 5444 | 8936 | 7598 |
| RAN_+_131356438.23-P1P2_02 | 11853 | 15358 | 15046 |
| RAN_+_131356438.23-P1P2_03 | 5056 | 6816 | 6698 |
| RAN_+_131356438.23-P1P2_04 | 6001 | 14870 | 11409 |
| RAN_+_131356438.23-P1P2_12 | 7627 | 25349 | 16172 |
| RPL9_+_39460483.23-P1P2_00 | 10355 | 1014 | 1141 |
| RPL9_+_39460483.23-P1P2_01 | 4886 | 1238 | 1108 |
| RPL9_+_39460483.23-P1P2_04 | 5237 | 4118 | 3975 |
| RPL9_+_39460483.23-P1P2_05 | 4950 | 2355 | 2217 |
| RPL9_+_39460483.23-P1P2_07 | 7336 | 9339 | 7867 |
| RPS14_+_149829238.23-P1P2_00 | 11846 | 2984 | 3190 |
| RPS14_+_149829238.23-P1P2_01 | 4954 | 1385 | 1474 |
| RPS14_+_149829238.23-P1P2_02 | 11519 | 5538 | 5497 |
| RPS14_+_149829238.23-P1P2_04 | 9244 | 12547 | 9641 |
| RPS14_+_149829238.23-P1P2_08 | 4488 | 17976 | 11681 |
| RPS14_+_149829238.23-P1P2_13 | 7137 | 12082 | 9567 |
| RPS15_−_1438413.23-P1P2_00 | 6757 | 3376 | 2912 |
| RPS15_−_1438413.23-P1P2_01 | 9713 | 39345 | 23866 |
| RPS15_−_1438413.23-P1P2_02 | 5051 | 3548 | 3113 |
| RPS15_−_1438413.23-P1P2_07 | 6337 | 4631 | 3595 |
| RPS15_−_1438413.23-P1P2_12 | 4661 | 19991 | 12257 |
| RPS18_+_33239917.23-P1P2_00 | 6212 | 1535 | 1556 |
| RPS18_+_33239917.23-P1P2_01 | 5202 | 2571 | 2658 |
| RPS18_+_33239917.23-P1P2_02 | 5486 | 3757 | 3404 |
| RPS18_+_33239917.23-P1P2_04 | 5132 | 13186 | 9728 |
| RPS18_+_33239917.23-P1P2_08 | 8535 | 13839 | 11101 |
| SEC61A1_−_127771295.23-P1_00 | 11429 | 2025 | 2151 |
| SEC61A1_−_127771295.23-P1_01 | 5308 | 4229 | 4006 |
| SEC61A1_−_127771295.23-P1_02 | 9991 | 4238 | 4030 |
| SEC61A1_−_127771295.23-P1_03 | 5904 | 3563 | 3530 |
| SEC61A1_−_127771295.23-P1_04 | 5081 | 10772 | 7999 |
| TUBB_+_30688126.23-P1_00 | 13570 | 1125 | 2722 |
| TUBB_+_30688126.23-P1_01 | 7125 | 962 | 1319 |
| TUBB_+_30688126.23-P1_03 | 4751 | 1221 | 1680 |
| TUBB_+_30688126.23-P1_06 | 6235 | 1158 | 1983 |
| TUBB_+_30688126.23-P1_10 | 7085 | 12737 | 9877 |
| non-targeting_00001 | 10415 | 31944 | 18946 |
| non-targeting_00028 | 8871 | 35652 | 20289 |
| non-targeting_00054 | 12360 | 49855 | 29818 |
| non-targeting_00089 | 10841 | 44919 | 27748 |
| non-targeting_00217 | 10286 | 42962 | 25185 |
| non-targeting_00283 | 8188 | 27936 | 18547 |
| non-targeting_00406 | 9974 | 39839 | 24099 |
| non-targeting_00527 | 6840 | 27634 | 16865 |
| non-targeting_00802 | 7096 | 27842 | 16759 |
| non-targeting_01040 | 0 | 0 | 0 |

TABLE 4
Perturb-seq sgRNA sequences and pooled growth phenotypes (γ and relative
activity).

| | | | | | | relative | |
|---|---|---|---|---|---|---|---|
| | | SEQ | | | | _act- | relative_ |
| | | ID | | | | ivity_ | activity |
| | Sequence | NO: | Gene | gamma_day5 | gamma_day10 | day5 | _day10 |
| ALDOA_ + _30077 | GGTCACCA | 156 | ALDOA | −0.412746257 | −0.366468568 | 1 | 1 |
| 139.23-P1P2_00 | GGACCCCT | | | | | | |
| | TCTG | | | | | | |
| ALDOA_ + _30077 | GGTCACCA | 157 | ALDOA | −0.396686909 | −0.348503022 | 0.96109 | 0.95097657 |
| 139.23-P1P2_06 | GGATCCCT | | | | | 1475 | |
| | TCTG | | | | | | |
| ALDOA_ + _30077 | GGTCACCA | 158 | ALDOA | −0.360892365 | −0.335059043 | 0.87436 | 0.91429135 |
| 139.23-P1P2_07 | GGCCCCCT | | | | | 8595 | 3 |
| | TCTG | | | | | | |
| ALDOA_ + _30077 | GGTCACCA | 160 | ALDOA | 0.017063022 | −0.000220283 | −0.04134 | 0.00060109 |
| 139.23-P1P2_13 | GGACCCCT | | | | | 0221 | 6 |
| | TTTG | | | | | | |
| ALDOA_ + _30077 | GGTCACCA | 159 | ALDOA | −0.175243431 | −0.156611393 | 0.42457 | 0.42735286 |
| 139.23-P1P2_14 | GGACCGCT | | | | | 9093 | 6 |
| | TCTG | | | | | | |
| ATP5E_ − | GGTGTCCA | 151 | ATP5E | −0.176898232 | −0.224723052 | 1 | 1 |
| _57607036.23- | GGGGCACT | | | | | | |
| P1P2_00 | CTGT | | | | | | |
| ATP5E_ − | GGTGTCCT | 152 | ATP5E | −0.209657934 | −0.228373078 | 1.18518 | 1.01624233 |
| _57607036.23- | GGGGCACT | | | | | 9542 | 1 |
| P1P2_01 | CTGT | | | | | | |
| ATP5E_ − | GGTGTCCA | 154 | ATP5E | −0.097932574 | −0.120406413 | 0.55360 | 0.53579911 |
| _57607036.23- | GGAGCACT | | | | | 9686 | 6 |
| P1P2_04 | CTGT | | | | | | |
| ATP5E_ − | GGTGTCCA | 155 | ATP5E | −0.012329915 | −0.035061828 | 0.06970 | 0.15602239 |
| _57607036.23- | GGGGCACT | | | | | 0615 | |
| P1P2_14 | GTGT | | | | | | |
| ATP5E_ − | GGTGTCCA | 153 | ATP5E | −0.161607088 | −0.165585326 | 0.91355 | 0.73684174 |
| _57607036.23- | GGGGCGCT | | | | | 9656 | 6 |
| P1P2_16 | CTGT | | | | | | |
| BCR_ + _2352309 | GCGCGCGG | 125 | BCR | −0.84255285 | −0.506782463 | 1 | 1 |
| 2.23-P1P2_00 | GGCCCGTC | | | | | | |
| | TCAG | | | | | | |
| BCR_ + _2352309 | GCGCGCGG | 127 | BCR | −0.740052021 | −0.418039669 | 0.87834 | 0.82488976 |
| 2.23-P1P2_04 | AGCCCGTC | | | | | 4926 | 8 |
| | TCAG | | | | | | |
| BCR_ + _2352309 | GCGCGCGG | 128 | BCR | −0.353548555 | −0.224691524 | 0.41961 | 0.44336878 |
| 2.23-P1P2_05 | CGCCCGTC | | | | | 588 | 2 |
| | TCAG | | | | | | |
| BCR_ + _2352309 | GCGCGCGG | 126 | BCR | −0.870659636 | −0.486879508 | 1.03335 | 0.96072682 |
| 2.23-P1P2_07 | GGCTCGTC | | | | | 9078 | 8 |
| | TCAG | | | | | | |
| BCR_ + _2352309 | GCGCGCGG | 130 | BCR | −0.218335768 | −0.161526418 | 0.25913 | 0.31872929 |
| 2.23-P1P2_13 | GGCCCATC | | | | | 5991 | 6 |
| | TCAG | | | | | | |
| BCR_ + _2352309 | GCGCGCGG | 129 | BCR | −0.231407972 | −0.177296007 | 0.27465 | 0.34984637 |
| 2.23-P1P2_15 | GGCCCGTC | | | | | 0987 | 5 |
| | GCAG | | | | | | |
| CAD_ + _2744028 | GGCTGGAG | 20 | CAD | −0.77142522 | −0.684031023 | 1 | 1 |
| 0.23-P1P2_00 | AGAAGCC | | | | | | |
| | GGGCG | | | | | | |
| CAD_ + _2744028 | GGCTGGTG | 102 | CAD | −0.768748241 | −0.62669484 | 0.99652 | 0.91617897 |
| 0.23-P1P2_03 | AGAAGCC | | | | | 9827 | 2 |
| | GGGCG | | | | | | |
| CAD_ + _2744028 | GGCTGGAG | 104 | CAD | −0.397541377 | −0.314108526 | 0.51533 | 0.45920216 |
| 0.23-P1P2_06 | AGTAGCC | | | | | 3654 | 4 |
| | GGGCG | | | | | | |
| CAD_ + _2744028 | GGCTGGAG | 103 | CAD | −0.602640029 | −0.465864745 | 0.78120 | 0.68105791 |
| 0.23-P1P2_07 | CGAAGCC | | | | | 3432 | 9 |
| | GGGCG | | | | | | |
| CAD_ + _2744028 | GGCTGGAG | 105 | CAD | −0.186141893 | −0.164847938 | 0.24129 | 0.24099482 |
| 0.23-P1P2_13 | AGAAGCC | | | | | 6094 | 7 |
| | TGGCG | | | | | | |
| CDC23_ − | GACAGCCA | 18 | CDC23 | −1.176148271 | −0.65836999 | 1 | 1 |
| _137548987.23- | CCGGGACC | | | | | | |
| P1P2_00 | ATGG | | | | | | |
| CDC23_ − | GACAGCTA | 98 | CDC23 | −1.086521687 | −0.570458445 | 0.92379 | 0.86647091 |
| _137548987.23- | CCGGGACC | | | | | 6526 | |
| P1P2_02 | ATGG | | | | | | |
| CDC23_ − | GACAGCCA | 100 | CDC23 | −0.888740688 | −0.536409046 | 0.75563 | 0.81475318 |
| _137548987.23- | ACGGGACC | | | | | 6607 | 3 |
| P1P2_04 | ATGG | | | | | | |
| CDC23_ − | GACAGCCA | 99 | CDC23 | −0.955927382 | −0.550062137 | 0.81276 | 0.83549090 |
| _137548987.23- | TCGGGACC | | | | | 0947 | 2 |
| P1P2_08 | ATGG | | | | | | |
| CDC23_ − | GACAGCCA | 101 | CDC23 | −0.274524181 | −0.201768195 | 0.23340 | 0.30646627 |
| _137548987.23- | CCGGGACC | | | | | 9501 | |
| P1P2_11 | ACGG | | | | | | |
| COX11_ + _53045 | GGCTCTGG | 141 | COX11 | −0.181673555 | −0.298760116 | 1 | 1 |
| 977.23-P1P2_00 | CGTCCTGG | | | | | | |
| | ATGG | | | | | | |
| COX11_ + _53045 | GGCTCTGT | 142 | COX11 | −0.171909541 | −0.287459131 | 0.94625 | 0.96217371 |
| 977.23-P1P2_03 | CGTCCTGG | | | | | 5175 | 7 |
| | ATGG | | | | | | |
| COX11_ + _53045 | GGCTCTGG | 143 | COX11 | −0.149463107 | −0.257412775 | 0.82270 | 0.86160354 |
| 977.23-P1P2_04 | CGCCCTGG | | | | | 1508 | 4 |
| | ATGG | | | | | | |
| COX11_ + _53045 | GGCTCTGG | 144 | COX11 | −0.055498122 | −0.107956558 | 0.30548 | 0.36134862 |
| 977.23-P1P2_05 | CGTCTTGG | | | | | 2668 | 8 |
| | ATGG | | | | | | |
| COX11_ + _53045 | GGCTCTGG | 145 | COX11 | −0.124745894 | −0.111555757 | 0.68664 | 0.37339574 |
| 977.23-P1P2_10 | CGTCCCGG | | | | | 8609 | 8 |
| | ATGG | | | | | | |
| DBR1_ + _137893 | GTTTGCAG | 78 | DBR1 | −0.455632712 | −0.489343933 | 1 | 1 |
| 744.23-P1P2_00 | GAGTCTAC | | | | | | |
| | ACCC | | | | | | |
| DBR1_ + _137893 | GATTGCAG | 79 | DBR1 | −0.5119081 | −0.547426521 | 1.12351 | 1.11869481 |
| 744.23-P1P2_01 | GAGTCTAC | | | | | 042 | 5 |
| | ACCC | | | | | | |
| DBR1_ + _137893 | GTTTGCAG | 81 | DBR1 | −0.310235296 | −0.309396024 | 0.68088 | 0.63226700 |
| 744.23-P1P2_05 | GAGTGTAC | | | | | 8988 | 7 |
| | ACCC | | | | | | |
| DBR1_ + _137893 | GTTTGCAG | 80 | DBR1 | −0.516738373 | −0.467972494 | 1.13411 | 0.95632634 |
| 744.23-P1P2_07 | GGGTCTAC | | | | | 1663 | 3 |
| | ACCC | | | | | | |
| DBR1_ + _137893 | GTTTGCAG | 82 | DBR1 | −0.035293825 | −0.052607373 | 0.07746 | 0.10750592 |
| 744.23-P1P2_08 | TAGTCTAC | | | | | 113 | 7 |
| | ACCC | | | | | | |
| DUT_ + _4862441 | GCGAGCGA | 110 | DUT | −0.792905177 | −0.645747334 | 1 | 1 |
| 1.23-P1P2_00 | GGAGACC | | | | | | |
| | ACCGG | | | | | | |
| DUT_ + _4862441 | GCCAGCGA | 111 | DUT | −0.792381209 | −0.603170546 | 0.99933 | 0.93406587 |
| 1.23-P1P2_01 | GGAGACC | | | | | 918 | 2 |
| | ACCGG | | | | | | |
| DUT_ + _4862441 | GCGAGCGA | 113 | DUT | −0.71971485 | −0.499796619 | 0.90769 | 0.7739817 |
| 1.23-P1P2_07 | GGAGCCC | | | | | 3468 | |
| | ACCGG | | | | | | |
| DUT_ + _4862441 | GCGAGCGA | 112 | DUT | −0.772472074 | −0.586165942 | 0.97423 | 0.90773265 |
| 1.23-P1P2_08 | GGAGGCC | | | | | 0079 | 5 |
| | ACCGG | | | | | | |
| DUT_ + _4862441 | GCGAGCGA | 114 | DUT | −0.386398362 | −0.319585061 | 0.48731 | 0.49490728 |
| 1.23-P1P2_10 | GGAGACC | | | | | 9761 | 7 |
| | AACGG | | | | | | |
| EIF2S1_ − | GAGCGAAG | 136 | EIF2S1 | −0.904664361 | −0.515496826 | 1 | 1 |
| _67827080.23- | CGCACGCT | | | | | | |
| P1P2_00 | GAGG | | | | | | |
| EIF2S1_ − | GAGCGAAA | 139 | EIF2S1 | −0.446658521 | −0.303163507 | 0.49372 | 0.58809965 |
| _67827080.23- | CGCACGCT | | | | | 8438 | 7 |
| P1P2_01 | GAGG | | | | | | |
| EIF2S1_ − | GAGCGCAG | 138 | EIF2S1 | −0.728758604 | −0.455577923 | 0.80555 | 0.88376474 |
| _67827080.23- | CGCACGCT | | | | | 6884 | 8 |
| P1P2_02 | GAGG | | | | | | |
| EIF2S1_ − | GAGCGAAG | 137 | EIF2S1 | −0.668831037 | −0.363481208 | 0.73931 | 0.70510852 |
| _67827080.23- | CGCGCGC | | | | | 4011 | 7 |
| P1P2_06 | TGAGG | | | | | | |
| EIF2S1_ − | GAGCGAAG | 140 | EIF2S1 | −0.083069099 | −0.040040492 | 0.09182 | 0.07767359 |
| _67827080.23- | CGCTCGCT | | | | | 3114 | 6 |
| P1P2_07 | GAGG | | | | | | |
| GATA1_ − | GTGAGCTT | 119 | GATA1 | −0.962732023 | −0.615305642 | 1 | 1 |
| _48645022.23- | GCCACATC | | | | | | |
| P1P2_00 | CCCA | | | | | | |
| GATA1_ − | GTGCGCTT | 120 | GATA1 | −0.985479206 | −0.625910566 | 1.02362 | 1.01723521 |
| _48645022.23- | GCCACATC | | | | | 7741 | 3 |
| P1P2_03 | CCCA | | | | | | |
| GATA1_ − | GTGAGCTT | 121 | GATA1 | −0.931261986 | −0.603230764 | 0.96731 | 0.98037580 |
| _48645022.23- | ACCACATC | | | | | 1738 | 5 |
| P1P2_04 | CCCA | | | | | | |
| GATA1_ − | GTGAGCTT | 123 | GATA1 | −0.507256622 | −0.348632235 | 0.52689 | 0.56660009 |
| _48645022.23- | GCGACATC | | | | | 2853 | 5 |
| P1P2_06 | CCCA | | | | | | |
| GATA1_ − | GTGAGCTT | 122 | GATA1 | −0.763232435 | −0.489322207 | 0.79277 | 0.79525064 |
| _48645022.23- | TCCACATC | | | | | 7654 | 2 |
| P1P2_08 | CCCA | | | | | | |
| GATA1 − | GTGAGCTT | 124 | GATA1 | −0.10842664 | −0.063270746 | 0.11262 | 0.10282815 |
| _48645022.23- | GCCACATC | | | | | 3905 | 8 |
| P1P2_12 | CGCA | | | | | | |
| GINS1_ − | GGACTAGA | 93 | GINS1 | −0.999320047 | −0.712462976 | 1 | 1 |
| _25388381.23- | ACGAAAG | | | | | | |
| P1P2_00 | GAGTG | | | | | | |
| GINS1_ − | GGACTATA | 96 | GINS1 | −0.746714755 | −0.479674571 | 0.74722 | 0.67326245 |
| _25388381.23- | ACGAAAGG | | | | | 2831 | 4 |
| P1P2_03 | AGTG | | | | | | |
| GINS1_ − | GGACTAGA | 95 | GINS1 | −0.979441588 | −0.654830488 | 0.98010 | 0.91910809 |
| _25388381.23- | ACGGAAG | | | | | 8015 | 5 |
| P1P2_06 | GAGTG | | | | | | |
| GINS1_ − | GGACTAGA | 94 | GINS1 | −1.038746197 | −0.672280776 | 1.03945 | 0.943601 |
| _25388381.23- | GCGAAAG | | | | | 2976 | |
| P1P2_08 | GAGTG | | | | | | |
| GINS1_ − | GGACTAGA | 97 | GINS1 | −0.353499818 | −0.260924277 | 0.35374 | 0.36622854 |
| _25388381.23- | ACGAAAG | | | | | 0345 | 2 |
| P1P2_14 | GAGCG | | | | | | |
| GNB2L1_ + _1806 | GTGCAAGG | 55 | GNB2L1 | −0.290807004 | −0.305387449 | 1 | 1 |
| 70873.23- | CGGCGGC | | | | | | |
| P1P2_00 | AGGAG | | | | | | |
| GNB2L1_ + _1806 | GTGCAAGA | 59 | GNB2L1 | −0.143812202 | −0.127266579 | 0.49452 | 0.41673808 |
| 70873.23- | CGGCGGC | | | | | 7986 | |
| P1P2_02 | AGGAG | | | | | | |
| GNB2L1_ + _1806 | GTGCAAGG | 58 | GNB2L1 | −0.380032091 | −0.273258144 | 1.30681 | 0.89479166 |
| 70873.23- | CGGGGGC | | | | | 8906 | 6 |
| P1P2_07 | AGGAG | | | | | | |
| GNB2L1_ + _1806 | GTGCAAGG | 56 | GNB2L1 | −0.306071563 | −0.31551831 | 1.05249 | 1.03317379 |
| 70873.23- | TGGCGGC | | | | | 0343 | 4 |
| P1P2_08 | AGGAG | | | | | | |
| GNB2L1_ + _1806 | GTGCAAGG | 57 | GNB2L1 | −0.20971562 | −0.207391481 | 0.72115 | 0.67910937 |
| 70873.23- | CGGCGGC | | | | | 0513 | 9 |
| P1P2_13 | GGGAG | | | | | | |
| HSPA5_ + _12800 | GAGCCGAG | 88 | HSPA5 | −0.747632216 | −0.427181596 | 1 | 1 |
| 3624.23- | TAGGCGA | | | | | | |
| P1P2_00 | CGGTG | | | | | | |
| HSPA5_ + _12800 | GAACCGAG | 91 | HSPA5 | −0.637327036 | −0.374808011 | 0.85246 | 0.87739737 |
| 3624.23- | TAGGCGAC | | | | | 0638 | 7 |
| P1P2_01 | GGTG | | | | | | |
| HSPA5_ + _12800 | GAGCCGAG | 89 | HSPA5 | −0.754402152 | −0.422480889 | 1.00905 | 0.98899599 |
| 3624.23- | AAGGCGA | | | | | 5169 | 9 |
| P1P2_04 | CGGTG | | | | | | |
| HSPA5_ + _12800 | GAGCCGAG | 92 | HSPA5 | −0.119163968 | −0.098351611 | 0.15938 | 0.23023372 |
| 3624.23- | TAGACGAC | | | | | 8487 | 8 |
| P1P2_06 | GGTG | | | | | | |
| HSPA5_ + _12800 | GAGCCGAG | 90 | HSPA5 | −0.75656582 | −0.44349394 | 1.01194 | 1.03818597 |
| 3624.23- | TGGGCGA | | | | | 9195 | 1 |
| P1P2_08 | CGGTG | | | | | | |
| HSPA9_ − | GGAGCTGC | 131 | HSPA9 | −0.949975554 | −0.589152811 | 1 | 1 |
| _137911079.23- | GCGATGC | | | | | | |
| P1P2_00 | GGTGG | | | | | | |
| HSPA9_ − | GGAGTTGC | 133 | HSPA9 | −0.650398211 | −0.402698763 | 0.68464 | 0.68352175 |
| _137911079.23- | GCGATGCG | | | | | 7314 | 4 |
| P1P2_02 | GTGG | | | | | | |
| HSPA9_ − | GGAGCTGC | 135 | HSPA9 | −0.200474473 | −0.165716053 | 0.21103 | 0.28127855 |
| _137911079.23- | GCAATGCG | | | | | 1192 | 7 |
| P1P2_04 | GTGG | | | | | | |
| HSPA9_ − | GGAGCTGC | 132 | HSPA9 | −0.810949638 | −0.503573798 | 0.85365 | 0.85474224 |
| _137911079.23- | GGGATGC | | | | | 3165 | 7 |
| P1P2_07 | GGTGG | | | | | | |
| HSPA9_ − | GGAGCTGC | 134 | HSPA9 | −0.358229634 | −0.276176791 | 0.37709 | 0.46876936 |
| _137911079.23- | TCGATGCG | | | | | 3529 | 8 |
| P1P2_08 | GTGG | | | | | | |
| HSPE1_ + _19836 | GGAGACTC | 9 | HSPE1 | −0.701840637 | −0.567192606 | 1 | 1 |
| 5089.23- | GCAGTCCG | | | | | | |
| P1P2_00 | GCCC | | | | | | |
| HSPE1_ + _19836 | GGAGACAC | 65 | HSPE1 | −0.615974016 | −0.483391078 | 0.87765 | 0.85225207 |
| 5089.23- | GCAGTCCG | | | | | 5102 | 9 |
| P1P2_01 | GCCC | | | | | | |
| HSPE1_ + _19836 | GGAGACTG | 67 | HSPE1 | −0.436529138 | −0.356453563 | 0.62197 | 0.62845241 |
| 5089.23- | GCAGTCCG | | | | | 7575 | 5 |
| P1P2_02 | GCCC | | | | | | |
| HSPE1_ + _19836 | GGTGACTC | 66 | HSPE1 | −0.642742797 | −0.46501262 | 0.91579 | 0.81984958 |
| 5089.23- | GCAGTCCG | | | | | 5927 | |
| P1P2_03 | GCCC | | | | | | |
| HSPE1_ + _19836 | GGAGACTC | 68 | HSPE1 | −0.208819998 | −0.196531939 | 0.29753 | 0.34649947 |
| 5089.23- | GCAGTCCT | | | | | 1928 | 3 |
| P1P2_14 | GCCC | | | | | | |
| MTOR_ + _11322 | GGGCAGGG | 146 | MTOR | −0.687219844 | −0.561792171 | 1 | 1 |
| 547.23-P1P2_00 | GGCCTGA | | | | | | |
| | AGCGG | | | | | | |
| MTOR_ + _11322 | GGGCAGGG | 148 | MTOR | −0.431253329 | −0.344754014 | 0.62753 | 0.61366824 |
| 547.23-P1P2_05 | GGCTTGA | | | | | 3289 | 2 |
| | AGCGG | | | | | | |
| MTOR_ + _11322 | GGGCAGGG | 149 | MTOR | −0.393307519 | −0.298854973 | 0.57231 | 0.53196713 |
| 547.23-P1P2_06 | GGGCTGA | | | | | 6882 | 8 |
| | AGCGG | | | | | | |
| MTOR_ + _11322 | GGGCAGGG | 147 | MTOR | −0.58933856 | −0.479851388 | 0.85756 | 0.85414395 |
| 547.23-P1P2_07 | GGTCTGA | | | | | 9183 | 7 |
| | AGCGG | | | | | | |
| MTOR_ + _11322 | GGGCAGGG | 150 | MTOR | −0.304808003 | −0.232661121 | 0.44353 | 0.41414090 |
| 547.23-P1P2_10 | GGCCTGA | | | | | 7837 | 9 |
| | AGCAG | | | | | | |
| POLR1D_ + _2819 | GGGAAGCA | 11 | POLR1D | −0.75939328 | −0.621320058 | 1 | 1 |
| 6016.23-P1_00 | AGGACCG | | | | | | |
| | ACCGA | | | | | | |
| POLR1D_ + _2819 | GGGAAGCC | 76 | POLR1D | −0.694457525 | −0.54164837 | 0.91448 | 0.87177029 |
| 6016.23-P1_01 | AGGACCG | | | | | 9952 | 4 |
| | ACCGA | | | | | | |
| POLR1D_ + _2819 | GGTAAGCA | 75 | POLR1D | −0.847116707 | −0.683333455 | 1.11551 | 1.09980910 |
| 6016.23-P1_03 | AGGACCG | | | | | 7781 | 2 |
| | ACCGA | | | | | | |
| POLR1D_ + _2819 | GGGAAGCA | 77 | POLR1D | −0.301916652 | −0.242974878 | 0.39757 | 0.39106234 |
| 6016.23-P1_07 | AGGAGCG | | | | | 6144 | 4 |
| | ACCGA | | | | | | |
| POLR1D_ + _2819 | GGGAAGCA | 74 | POLR1D | −0.808813476 | −0.631725462 | 1.06507 | 1.01674725 |
| 6016.23-P1_08 | GGGACCG | | | | | 8526 | 2 |
| | ACCGA | | | | | | |
| POLR2H_ + _1840 | GGGGCCAC | 28 | POLR2H | −1.149173044 | −0.639125666 | 1 | 1 |
| 81251.23- | GAGAGCA | | | | | | |
| P1P2_00 | GCAGA | | | | | | |
| POLR2H_ + _1840 | GGGGCCAC | 118 | POLR2H | −0.189410601 | −0.142550442 | 0.16482 | 0.22303977 |
| 81251.23- | GAGTGCA | | | | | 3394 | 1 |
| P1P2_07 | GCAGA | | | | | | |
| POLR2H_ + _1840 | GGGGCCAC | 116 | POLR2H | −0.52081984 | −0.333960225 | 0.45321 | 0.5225267 |
| 81251.23- | GCGAGCA | | | | | 2719 | |
| P1P2_08 | GCAGA | | | | | | |
| POLR2H_ + _1840 | GGGGCCAC | 115 | POLR2H | −0.996608089 | −0.546680283 | 0.86723 | 0.85535648 |
| 81251.23- | GAGAGCA | | | | | 9354 | 5 |
| P1P2_11 | GCGGA | | | | | | |
| POLR2H_ + _1840 | GGGGCCAC | 117 | POLR2H | −0.351637117 | −0.229753975 | 0.30599 | 0.35948169 |
| 81251.23- | GAGAGCA | | | | | 1442 | 1 |
| P1P2_12 | GGAGA | | | | | | |
| RAN_ + _1313564 | GGCGGTCG | 69 | RAN | −0.197388026 | −0.16148153 | 1 | 1 |
| 38.23-P1P2_00 | CTGCGCTT | | | | | | |
| | AGGG | | | | | | |
| RAN_ + _1313564 | GGCGGCCG | 70 | RAN | −0.231594252 | −0.204134533 | 1.17329 | 1.26413548 |
| 38.23-P1P2_02 | CTGCGCTT | | | | | 4328 | 5 |
| | AGGG | | | | | | |
| RAN_ + _1313564 | GGGGGTCG | 71 | RAN | −0.21619271 | −0.196985686 | 1.09526 | 1.21986511 |
| 38.23-P1P2_03 | CTGCGCTT | | | | | 7598 | 9 |
| | AGGG | | | | | | |
| RAN_ + _1313564 | GGCGGTCG | 72 | RAN | −0.08590181 | −0.087210569 | 0.43519 | 0.54006528 |
| 38.23-P1P2_04 | CGGCGCTT | | | | | 2609 | 5 |
| | AGGG | | | | | | |
| RAN_ + _1313564 | GGCGGTCG | 73 | RAN | −0.046548562 | −0.034259141 | 0.23582 | 0.21215516 |
| 38.23-P1P2_12 | CTGCGCTT | | | | | 2621 | 9 |
| | AGGT | | | | | | |
| RPL9_ + _394604 | GGATGTTT | 5 | RPL9 | −1.113115402 | −0.669876545 | 1 | 1 |
| 83.23-P1P2_00 | CTGTGCTC | | | | | | |
| | GTGG | | | | | | |
| RPL9_ + _394604 | GGATGATT | 51 | RPL9 | −0.852800183 | −0.498432114 | 0.76613 | 0.74406563 |
| 83.23-P1P2_01 | CTGTGCTC | | | | | 8158 | 1 |
| | GTGG | | | | | | |
| RPL9_ + _394604 | GGATGTTT | 53 | RPL9 | −0.417072624 | −0.294201392 | 0.37468 | 0.43918748 |
| 83.23-P1P2_04 | CAGTGCTC | | | | | 9473 | 1 |
| | GTGG | | | | | | |
| RPL9_ + _394604 | GGATGTTT | 52 | RPL9 | −0.607331126 | −0.384814478 | 0.54561 | 0.57445581 |
| 83.23-P1P2_05 | CGGTGCTC | | | | | 3802 | 8 |
| | GTGG | | | | | | |
| RPL9_ + _394604 | GGATGTTT | 54 | RPL9 | −0.292421202 | −0.20731749 | 0.26270 | 0.30948611 |
| 83.23-P1P2_07 | CTGCGCTC | | | | | 5198 | 7 |
| | GTGG | | | | | | |
| RPS14_ + _14982 | GAGGCCCG | 45 | RPS14 | −0.790819103 | −0.499486864 | 1 | 1 |
| 9238.23- | GGCGCGA | | | | | | |
| P1P2_00 | CAATC | | | | | | |
| RPS14_ + _14982 | GAGACCCG | 46 | RPS14 | −0.754840524 | −0.480690282 | 0.95450 | 0.96236821 |
| 9238.23- | GGCGCGA | | | | | 4666 | 5 |
| P1P2_01 | CAATC | | | | | | |
| RPS14_ + _14982 | GAGGCCCT | 47 | RPS14 | −0.584450961 | −0.38292411 | 0.73904 | 0.76663499 |
| 9238.23- | GGCGCGAC | | | | | 5072 | 6 |
| P1P2_02 | AATC | | | | | | |
| RPS14_ + _14982 | GAGGCCCG | 48 | RPS14 | −0.302459804 | −0.195757634 | 0.38246 | 0.39191748 |
| 9238.23- | CGCGCGA | | | | | 3957 | 2 |
| P1P2_04 | CAATC | | | | | | |
| RPS14_ + _14982 | GAGGCCCG | 50 | RPS14 | 0.027378614 | −0.000610864 | −0.03462 | 0.00122298 |
| 9238.23- | GGCTCGAC | | | | | 0577 | 3 |
| P1P2_08 | AATC | | | | | | |
| RPS14_ + _14982 | GAGGCCCG | 49 | RPS14 | −0.211938981 | −0.155918093 | 0.26799 | 0.31215654 |
| 9238.23- | GGCGCGA | | | | | 9319 | 4 |
| P1P2_13 | CAGTC | | | | | | |
| RPS15_ − | GACCAAAG | 60 | RPS15 | −0.621219313 | −0.375985289 | 1 | 1 |
| _1438413.23- | CGATCTCT | | | | | | |
| P1P2_00 | TCTG | | | | | | |
| RPS15_ − | GACCAAAC | 64 | RPS15 | 0.006615792 | 0.001422135 | −0.01064 | −0.00378242 |
| _1438413.23- | CGATCTCT | | | | | 9689 | 2 |
| P1P2_01 | TCTG | | | | | | |
| RPS15_ − | GACCAAGG | 62 | RPS15 | −0.492192054 | −0.314547174 | 0.79229 | 0.83659436 |
| _1438413.23- | CGATCTCT | | | | | 9988 | 5 |
| P1P2_02 | TCTG | | | | | | |
| RPS15_ − | GACCAAAG | 61 | RPS15 | −0.522078249 | −0.307411328 | 0.84040 | 0.81761530 |
| _1438413.23- | CGGTCTCT | | | | | 8916 | 7 |
| P1P2_07 | TCTG | | | | | | |
| RPS15_ − | GACCAAAG | 63 | RPS15 | 0.031097436 | 0.011728108 | 0.05005 | 0.03119299 |
| _1438413.23- | CGATCTCT | | | | | 8707 | 6 |
| P1P2_12 | TGTG | | | | | | |
| RPS18_ + _33239 | GCTGCGAT | 40 | RPS18 | −0.81693013 | −0.502954192 | 1 | 1 |
| 917.23-P1P2_00 | GCCGCTGG | | | | | | |
| | ATCA | | | | | | |
| RPS18_ + _33239 | GCTGCAAT | 41 | RPS18 | −0.559807511 | −0.377943894 | 0.68525 | 0.75144794 |
| 917.23-P1P2_01 | GCCGCTGG | | | | | 7516 | 5 |
| | ATCA | | | | | | |
| RPS18_ + _33239 | GCTGGGAT | 42 | RPS18 | −0.489757084 | −0.319123483 | 0.59950 | 0.63449810 |
| 917.23-P1P2_02 | GCCGCTGG | | | | | 9145 | 9 |
| | ATCA | | | | | | |
| RPS18_ + _33239 | GCTGCGAT | 44 | RPS18 | −0.086970673 | −0.080675056 | 0.10646 | 0.16040239 |
| 917.23-P1P2_04 | CCCGCTGG | | | | | 0357 | 4 |
| | ATCA | | | | | | |
| RPS18_ + _33239 | GCTGCGAT | 43 | RPS18 | −0.222819542 | −0.163692215 | 0.27275 | 0.32546147 |
| 917.23-P1P2_08 | TCCGCTGG | | | | | 2263 | 9 |
| | ATCA | | | | | | |
| SEC61A1_ − | GGCACTGA | 83 | SEC61A1 | −0.920031125 | −0.562939966 | 1 | 1 |
| _127771295.23- | CGTGTCTC | | | | | | |
| P1_00 | TCGG | | | | | | |
| SEC61A1_ − | GGCACTGT | 85 | SEC61A1 | −0.419127675 | −0.291833272 | 0.45555 | 0.51840922 |
| _127771295.23- | CGTGTCTC | | | | | 8148 | 5 |
| P1_01 | TCGG | | | | | | |
| SEC61A1_ − | GGCGCTGA | 84 | SEC61A1 | −0.645088499 | −0.405507482 | 0.70115 | 0.72033877 |
| _127771295.23- | CGTGTCTC | | | | | 943 | |
| P1_02 | TCGG | | | | | | |
| SEC61A1_ − | GGTACTGA | 86 | SEC61A1 | −0.503132322 | −0.341926825 | 0.54686 | 0.60739483 |
| _127771295.23- | CGTGTCTC | | | | | 4458 | |
| P1_03 | TCGG | | | | | | |
| SEC61A1_ − | GGCACTGA | 87 | SEC61A1 | −0.153949391 | −0.115339075 | 0.16733 | 0.20488698 |
| _127771295.23- | AGTGTCTC | | | | | 0633 | 9 |
| P1_04 | TCGG | | | | | | |
| TUBB_ + _306881 | GCGGCAGG | 23 | TUBB | −0.897046625 | −0.699904772 | 1 | 1 |
| 26.23-P1_00 | AAGGTTCT | | | | | | |
| | GAGA | | | | | | |
| TUBB_ + _306881 | GCAGCAGG | 106 | TUBB | −0.92598755 | −0.611949447 | 1.03226 | 0.87433244 |
| 26.23-P1_01 | AAGGTTCT | | | | | 2454 | |
| | GAGA | | | | | | |
| TUBB_ + _306881 | GCGGCAGC | 108 | TUBB | −0.692568681 | −0.495872796 | 0.77205 | 0.70848609 |
| 26.23-P1_03 | AAGGTTCT | | | | | 4274 | 1 |
| | GAGA | | | | | | |
| TUBB_ + _306881 | GCGGCAGG | 107 | TUBB | −0.730802408 | −0.554446084 | 0.81467 | 0.79217360 |
| 26.23-P1_06 | ACGGTTCT | | | | | 6058 | 1 |
| | GAGA | | | | | | |
| TUBB_ + _306881 | GCGGCAGG | 109 | TUBB | −0.197799924 | −0.145078576 | 0.22050 | 0.20728330 |
| 26.23-P1_10 | AAGGTTC | | | | | 1274 | 7 |
| | AGAGA | | | | | | |

TABLE 5
Oligonucleotide sequences used in this study.

| | | | SEQ | |
|---|---|---|---|---|
| | | | ID | |
| Experiment | Oligo ID | Sequence | NO: | Notes |
| Constant region | constant_ | TAAGCTGGAAACAGCATAGCAAGCTCAAATAAGACTAGTTCG | 171 | |
| variants | region_1_fw | TTATCAACTTGAAAAAGTGGCACCGAGTCGGTGCTTTTTTTC | | |
| Constant region | constant_ | TCGAGAAAAAAAGCACCGACTCGGTGCCACTTTTTCAAGTTG | 172 | |
| variants | region_1_rv | ATAACGAACTAGTCTTATTTGAGCTTGCTATGCTGTTTCCAGC | | |
| Constant region | constant_ | TAAGCTGGAAACAGCATAGCAAGTTCAAATAAGGCTAGTCCG | 173 | |
| variants | region_2_fw | TTATGTACTTCAAAAAGTGGCACCGAGTCGGTGCTTTTTTTC | | |
| Constant region | constant_ | TCGAGAAAAAAAGCACCGACTCGGTGCCACTTTTTGAAGTAC | 174 | |
| variants | region_2_rv | ATAACGGACTAGCCTTATTTGAACTTGCTATGCTGTTTCCAGC | | |
| Constant region | constant_ | TAAGCTGGAAACAGCATAGCGAGTTCAAATAAGGCTCGTCCG | 175 | |
| variants | region_3_fw | TTATCCACTTGAAAAAGTGGCACCGAGTCGGTGCTTTTTTTC | | |
| Constant region | constant_ | TCGAGAAAAAAAGCACCGACTCGGTGCCACTTTTTCAAGTGG | 176 | |
| variants | region_3_rv | ATAACGGACGAGCCTTATTTGAACTCGCTATGCTGTTTCCAGC | | |
| Constant region | constant_ | TAAGCTGGAAACAGCATAGCAAGTTCAAATAAAGTTAATCTG | 177 | |
| variants | region_4_fw | TTATCAACTCGAAAGAGIGGCACCGAGTCGGIGCTTTTTTTC | | |
| Constant region | constant_ | TCGAGAAAAAAAGCACCGACTCGGTGCCACTCTTTCGAGTTG | 178 | |
| variants | region_4_rv | ATAACAGATTAACTTTATTTGAACTTGCTATGCTGTTTCCAGC | | |
| Constant region | constant_ | TAAGCTGGAAACAGCATAGCAAGTTCAAATAAGGCTAGCCCG | 179 | |
| variants | region_5_fw | TTATGAACTTGAAAAAGTGGCACCGAGTCGGTGCTTTTTTTC | | |
| Constant region | constant_ | TCGAGAAAAAAAGCACCGACTCGGTGCCACTTTTTCAAGTTC | 180 | |
| variants | region_5_rv | ATAACGGGCTAGCCTTATTTGAACTTGCTATGCTGTTTCCAGC | | |
| Constant region | constant_ | TAAGCTGGAAACAGCATAGCAAGTTCAAATAAGGCTAGTCCG | 181 | |
| variants | region_6_fw | TTATCAACTTGAAAAAGTGGCACCGGGGCGGTGCTTTTTTTC | | |
| Constant region | constant_ | TCGAGAAAAAAAGCACCGCCCCGGTGCCACTTTTTCAAGTTG | 182 | |
| variants | region_6_rv | ATAACGGACTAGCCTTATTTGAACTTGCTATGCTGTTTCCAGC | | |
| Constant region | constant_ | TAAGCTGGAAACAGCATAGCAAGTTCAAATATGGCTAGTCCG | 183 | |
| variants | region_7_fw | TTATCAACTTGAAAAAGTGGCACCGAGTCGGTGCTTTTTTTC | | |
| Constant region | constant_ | TCGAGAAAAAAAGCACCGACTCGGTGCCACTTTTTCAAGTTG | 184 | |
| variants | region_7_rv | ATAACGGACTAGCCATATTTGAACTTGCTATGCTGTTTCCAGC | | |
| Constant region | constant_ | TAAGCTGGAAACAGCATAGCAAGTTCAAATAAGGATATTCCG | 185 | |
| variants | region_8_fw | TTATCAAGTTGAAAAACTGGCACCGAGTCGGTGCTTTTTTTC | | |
| Constant region | constant_ | TCGAGAAAAAAAGCACCGACTCGGTGCCAGTTTTTCAACTTG | 186 | |
| variants | region_8_rv | ATAACGGAATATCCTTATTTGAACTTGCTATGCTGTTTCCAGC | | |
| Constant region | constant_ | TAAGCTGGAAACAGCATAGCAAGTTCAAATAAGGCTAGTCCG | 187 | |
| variants | region_9_fw | TTATCAACTTGAGAAAGTGGCACCGGGTCGGTGCTTTTTTTC | | |
| Constant region | constant_ | TCGAGAAAAAAAGCACCGACCCGGTGCCACTTTCTCAAGTTG | 188 | |
| variants | region_9_rv | ATAACGGACTAGCCTTATTTGAACTTGCTATGCTGTTTCCAGC | | |
| Constant region | constant_ | TAAGCTGGAAACAGCATAGCAAGTTCAAATAAGGCTAGTCCG | 189 | |
| variants | region_10_fw | TTATCAACTTGAAAAAGTGGCACCGCGTCGGTGCTTTTTTTC | | |
| Constant region | constant_ | TCGAGAAAAAAAGCACCGACGCGGTGCCACTTTTTCAAGTTG | 190 | |
| variants | region_10_rv | ATAACGGACTAGCCTTATTTGAACTTGCTATGCTGTTTCCAGC | | |
| DPH2 knockdown | DPH2_qPCR_fw | ACCTGGACGGAGTGTACGAG | 191 | |
| (CR variants) | | | | |
| DPH2 knockdown | DPH2_qPCR_rv | TCTCCCAATAGCTGGTCAGG | 192 | |
| (CR variants) | | | | |
| DPH2 knockdown | ACTB_qPCR_fw | GCTACGAGCTGCCTGACG | 193 | |
| (CR variants) | | | | |
| DPH2 knockdown | ACTB_qPCR_rv | GGCTGGAAGAGTGCCTCA | 194 | |
| (CR variants) | | | | |
| Illumina | oCRISPRi_seq_ | GTGTGTTTTGAGACTATAAGTATCCCTTGGAGAACCACCTTGT | 195 | |
| sequencing | V5 | TG | | |
| primer | | | | |
| Illumina | oCRISPRi_seq_ | CCACTTTTTCAAGTTGATAACGGACTAGCCTTATTTAAACTTG | 196 | |
| sequencing | V4_3′ | CTATGCTGT | | |
| primer | | | | |
| Constant region | oCRISPRi_PE_ | AATGATACGGCGACCACCGAGATCTACACGCACAAAAGGAA | 197 | |
| sequencing | constant_ | ACTCACCCT | | |
| library | region_ | | | |
| preparation | common_ | | | |
| | primer | | | |
| Constant region | oCRISPRi_PE_ | CAAGCAGAAGACGGCATACGAGATNNNNNNGTCTCGTGGG | 198 | NN |
| sequencing | constant_ | CTCGGAGATGTGTATAAGAGACAGGCCGCCTAATGGATCCTA | | NN |
| library | region | G | | NN |
| preparation | _indexing_ | | | denotes |
| | primer | | | 6- |
| | | | | base |
| | | | | pair |
| | | | | Tru |
| | | | | Seq |
| | | | | index |
| Perturb-seq | oBA503 | CAAGCAGAAGACGGCATACGAGATCAGCCTCGGTCTCGTGG | 199 | |
| sequencing | | GCTCGGAGATGTGTATAAGAGACAGGTGTTTTGAGACTATAA | | |
| library | | GTATCCCTTGGAGAACCACCTTGTTG | | |
| preparation | | | | |
| Perturb-seq | PCR_perturb- | AATGATACGGCGACCACCGAGATCTACAC | 200 | |
| sequencing | seq_P5 | | | |
| library | | | | |
| preparation | | | | |

TABLE 6
Ranking of sgRNA constant region mutations. The constant region “cr995”
corresponds to the original, un-modified sequence. Each sequence begins
with the nucleotide immediately following the targeting sequence.

| | | SEQ | | Mean |
|---|---|---|---|---|
| | | ID | Muta- | relative |
| | Sequence | NO: | tion(s) | activity |
| cr748 | GTTTAAGAGCTAAGCTGGAAACAGCATAGCAAGTTTAAATAAGGCTAGT | 201 | U61C, | 1.14554678 |
| | CCGTTATCAACTCGAGAGAGTGGCACCGAGTCGGTGCT | | A64G, | 7 |
| | | | A66G | |
| cr289 | GTTTAAGAGCTAAGCTGGAAACAGCATAGCAAGTTTAAATAAGGCTAGT | 202 | U61G, | 1.10155915 |
| | CCGTTATCAACTGGAAACAGTGGCACCGAGTCGGTGCT | | A66C | 3 |
| cr622 | GTTTAAGAGCTAAGCTGGAAACAGCATAGCAAGTTTAAATAAGGCTAGT | 203 | A58G, | 1.09945059 |
| | CCGTTATCAGCTGGAAACAGCGGCACCGAGTCGGTGCT | | U61G, | 1 |
| | | | A66C, | |
| | | | U69C | |
| cr772 | GTTTAAGAGCTAAGCTGGAAACAGCATAGCAAGTTTAAATAAGGCTAGT | 204 | U61C, | 1.09851461 |
| | CCGTTATCAACTCGAAAGAGTGGCACCGAGTCGGTGCT | | A66G | |
| cr532 | GTTTAAGAGCTAAGCTGGAAACAGCATAGCAAGTTTAAATAAGGCTAGT | 205 | U60G, | 1.09257901 |
| | CCGTTATCAACGTGAAAACGTGACTCCGAGTCGGAGTT | | A67C, | 7 |
| | | | G71A, | |
| | | | A73U, | |
| | | | U83A, | |
| | | | C85U | |
| cr961 | GTTTAAGAGCTAAGCTGGAAACAGCATAGCAAGTTTAAATAAGGCTAGT | 206 | U61C, | 1.08755170 |
| | CCGTTATCAACTCGAAAGAGTGCAACCGAGTCGGTTGT | | A66G, | 1 |
| | | | G71C, | |
| | | | C72A, | |
| | | | G84U, | |
| | | | C85G | |
| cr942 | GTTTAAGAGCTAAGCTGGAAACAGCATAGCAAGTTTAAATAAGGCTAGT | 207 | U55C, | 1.08746952 |
| | CCGTTACCAACTTGAACAAGTGGCACCGAGTCGGTGCT | | A65C | 3 |
| cr565 | GTTTAAGAGCTAAGCTGGAAACAGCATAGCAAGTTTAAATAAGGCTAGT | 208 | U60G, | 1.08457776 |
| | CCGTTATCAACGCGAAAGCGTGGCACCGAGTCGGTGCT | | U61C, | 5 |
| | | | A66G, | |
| | | | A67C | |
| cr925 | GTTTAAGAGCTAAGCTGGAAACAGCATAGCAAGTTTAAATAAGGCTAGT | 209 | A58U, | 1.08416233 |
| | CCGTTATCATCGAGAAATCGAGGCACCGAGTCGGTGCT | | U60G, | 9 |
| | | | U61A, | |
| | | | A66U, | |
| | | | A67C, | |
| | | | U69A | |
| cr234 | GTTTAAGAGCTAAGCTGGAAACAGCATAGCAAGTTTAAATAAGGCTAGT | 210 | U61C | 1.07543830 |
| | CCGTTATCAACTCGAAAAAGTGGCACCGAGTCGGTGCT | | | 9 |
| cr820 | GTTTAAGAGCTAAGCTGGAAACAGCATAGCAAGTTTAAATAAGGCTAGT | 211 | U61G, | 1.07186330 |
| | CCGTTATCAACTGGAGACAGTGGCACCGAGTCGGTGCT | | A64G, | 1 |
| | | | A66C | |
| cr936 | GTTTAAGAGCTAAGCTGGAAACAGCATAGCAAGTTTAAATAAGGCTAGT | 212 | G71U, | 1.07182960 |
| | CCGTTATCAACTTGAAAAAGTGTCACCGAGTCGGTGAT | | C85A | 8 |
| cr333 | GTTTAAGAGCTAAGCTGGAAACAGCATAGCAAGTTTAAATAAGGCTAGT | 213 | C59G, | 1.07174594 |
| | CCGTTATCAAGGTGAAAACCTGGCACCGAGTCGGTGCT | | U60G, | 6 |
| | | | A67C, | |
| | | | G68C | |
| cr156 | GTTTAAGAGCTAAGCTGGAAACAGCATAGCAAGTTTAAATAAGGCTAGT | 214 | U61A, | 1.07174467 |
| | CCGTTATCAACTAGAAATAGTGGCACCGAGTCGGTGCT | | A66U | |
| cr363 | GTTTAAGAGCTAAGCTGGAAACAGCATAGCAAGTTTAAATAAGGCTAGT | 215 | C59U, | 1.06984825 |
| | CCGTTATCAATTCGAAAGAATGGCACCGAGTCGGTGCT | | U61C, | 4 |
| | | | A66G, | |
| | | | G68A | |
| cr534 | GTTTAAGAGCTAAGCTGGAAACAGCATAGCAAGTTTAAATAAGGTTAAT | 216 | C44U, | 1.06923287 |
| | CCGTTATCAACTTGAAAAAGTGACACCGAGTCGGTGTT | | G47A, | 5 |
| | | | G71A, | |
| | | | C85U | |
| cr563 | GTTTAAGAGCTAAGCTGGAAACAGCATAGCAAGTTTAAATAAGGCTAGT | 217 | C59G, | 1.06815963 |
| | CCGTTATCAAGCTGAAAAGCTGGCACCGAGTCGGTGCT | | U60C, | 5 |
| | | | A67G, | |
| | | | G68C | |
| cr176 | GTTTAAGAGCTAAGCTGGAAACAGCATAGCAAGTTTAAATAAGGCTAGT | 218 | A58G, | 1.06722614 |
| | CCGTTATCAGCTTGAAAAAGCGGCACCGAGTCGGTGCT | | U69C | 8 |
| cr327 | GTTTAAGAGCTAAGCTGGAAACAGCATAGCAAGTTTAAATAAGGCTAGT | 219 | A63C | 1.06626839 |
| | CCGTTATCAACTTGCAAAAGTGGCACCGAGTCGGTGCT | | | 6 |
| cr360 | GTTTAAGAGCTAAGCTGGAAACAGCATAGCAAGTTTAAATAAGGCTAGT | 220 | C74U, | 1.0659663 |
| | CCGTTATCAACTTGAAAAAGTGGCATCGAGTCGATGCT | | G82A | |
| cr944 | GTTTAAGAGCTAAGCTGGAAACAGCATAGCAAGTTTAAATAAGGCTAGT | 221 | U61C, | 1.06526905 |
| | CCGTTATCAACTCGAAAGAGTGGTACCAAGTTGGTACT | | A66G, | 3 |
| | | | C72U, | |
| | | | G76A, | |
| | | | C80U, | |
| | | | G84A | |
| cr612 | GTTTAAGAGCTAAGCTGGATACAGCATAGCAAGTTTAAATAAGGCTAGT | 222 | A19U | 1.06437074 |
| | CCGTTATCAACTTGAAAAAGTGGCACCGAGTCGGTGCT | | | 1 |
| cr116 | GTTTAAGAGCTAAGCTGGAAACAGCATAGCAAGTTTAAATAAGGCTAGT | 223 | U60G, | 1.06400045 |
| | CCGTTATCAACGTGAAAACGTGGCACCGAGTCGGTGCT | | A67C | 9 |
| cr450 | GTTTAAGAGCTAAGCTGGAAACAGCATAGCAAGTTTAAATAAGGCTAGT | 224 | A64G, | 1.06386665 |
| | CCGTTATCAACTTGAGAAAGTGGCGCCGAGTCGGCGCT | | A73G, | 6 |
| | | | U83C | |
| cr567 | GTTTAAGAGCTAAGCTGGAAACAGCATAGCAAGTTTAAATAAGGCTAGT | 225 | A63C, | 1.06027433 |
| | CCGTTATCAACTTGCGAAAGTGGCACCGAGTCGGTGCT | | A64G | 5 |
| cr275 | GTTTAAGAGCTAAGCTGGAAACAGCATAGCAAGTTTAAATAAGGCTAGT | 226 | U60C, | 1.06001789 |
| | CCGTTATCAACCTGAAAAGGTGGGACAGAGTCTGTCCT | | A67G, | 9 |
| | | | C72G, | |
| | | | C75A, | |
| | | | G81U, | |
| | | | G84C | |
| cr488 | GTTTAAGAGCTAAGCTGGAAACAGCATAGCAAGTTTAAATAAGGCTAGT | 227 | C72G, | 1.05952873 |
| | CCGTTATCAACTTGAAAAAGTGGGACCGAGTCGGTCCT | | G84C | 1 |
| cr617 | GTTTAAGAGCTAAGCTGGAAACAGCATAGCAAGTTTAAATAAGGTTAAT | 228 | C44U, | 1.05837720 |
| | CCGTTATCAGCGTGAAAACGCGGCACCGAGTCGGTGCT | | G47A, | 1 |
| | | | A58G, | |
| | | | U60G, | |
| | | | A67C, | |
| | | | U69C | |
| cr022 | GTTTAAGTGCTAAGCTGGAAACAGCATAGCAAGTTTAAATAAGGCTAGT | 229 | A7U | 1.05753552 |
| | CCGTTATCAACTTGAAAAAGTGGCACCGAGTCGGTGCT | | | 5 |
| cr717 | GTTTAAGAGCTAAGCTGGAAACAGCATAGCAAGTTTAAATAAGGTTAAT | 230 | C44U, | 1.05639699 |
| | CCGTTATCAGCTTGAAAAAGCGGCACCGAGTCGGTGCT | | G47A, | 3 |
| | | | A58G, | |
| | | | U69C | |
| cr919 | GTTTAAGAGCTAAGCTGGAAACAGCATAGCAAGTTTAAATAAGGCTAGT | 231 | A58C, | 1.05607768 |
| | CCGTTATCACCTGGAAACAGGGGCACCGAGTCGGTGCT | | U61G, | 1 |
| | | | A66C, | |
| | | | U69G | |
| cr585 | GTTTAAGAGCTAAGCTGGAAACAGCATAGCAAGTTTAAATAAGGCTAGT | 232 | A73U, | 1.05576943 |
| | CCGTTATCAACTTGAAAAAGTGGCTCCGAGTCGGAGCT | | U83A | 9 |
| cr394 | GTTTAAGAGCTAAGCTGGAAACAGCATAGCAAGTTTAAATAAGGCTAGT | 233 | A66C | 1.05517587 |
| | CCGTTATCAACTTGAAACAGTGGCACCGAGTCGGTGCT | | | 4 |
| cr477 | GTTTAAGAGCTAAGCTGGAAACAGCATAGCAAGTTTAAATAAGGCTAGT | 234 | U55C | 1.05504247 |
| | CCGTTACCAACTTGAAAAAGTGGCACCGAGTCGGTGCT | | | 4 |
| cr380 | GTTTAAGAGCTAAGCTGGAAACAGCATAGCAAGTTTAAATAAGGCTAGT | 235 | A64G, | 1.05472264 |
| | CCGTTATCAACTTGAGAAAGTGGTACCGAGTCGGTACT | | C72U, | 6 |
| | | | G84A | |
| cr568 | GTTTAAGAGCTAAGCTGGAAACAGCATAGCAAGTTTAAATAAGGCTAGT | 236 | A58U, | 1.05448905 |
| | CCGTTATCATCGTGAAAACGAGGCAACGAGTCGTTGCT | | U60G, | 7 |
| | | | A67C, | |
| | | | U69A, | |
| | | | C74A, | |
| | | | G82U | |
| cr723 | GTTTAAGAGCTAAGCTGGAAACAGCATAGCAAGTTTAAATAAGGCTAGT | 237 | U60C, | 1.05376194 |
| | CCGTTATCAACCTGAAAAGGTGGCAGCGAGTCGCTGCT | | A67G, | 5 |
| | | | C74G, | |
| | | | G82C | |
| cr501 | GTTTAAGAGCTAAGCTGGAAACAGCATAGCAAGTTTAAATAAGGCTAGT | 238 | A64G, | 1.05248281 |
| | CCGTTATCAACTTGAGAAAGTGTCACCGAGTCGGTGAT | | G71U, | 9 |
| | | | C85A | |
| cr293 | GTTTAAGAGCTAAGCTGGAAACAGCATAGCAAGTTTAAATAAGGCTAGT | 239 | C72G, | 1.05198607 |
| | CCGTTATCAACTTGAAAAAGTGGGACCAAGTTGGTCCT | | G76A, | 8 |
| | | | C80U, | |
| | | | G84C | |
| cr549 | GTTTAAGAGCTAAGCTGGAAACAGCATAGCAAGTTTAAATAAGGCTAGT | 240 | U60C, | 1.05123166 |
| | CCGTTATCAACCTGAAAAGGTGGCACCGAGTCGGTGCT | | A67G | 2 |
| cr766 | GTTTAAGAGCTAAGCTGGAAACAGCATAGCAAGTTTAAATAAGGCTAGT | 241 | U60A, | 1.05088127 |
| | CCGTTATCAACATGAAAATGTGGCACCGAGTCGGTGCT | | A67U | 6 |
| cr602 | GTTTAAGAGCTAAGCTGGAAACAGCATAGCAAGTTTAAATAAGGCTAGT | 242 | A73G, | 1.04938568 |
| | CCGTTATCAACTTGAAAAAGTGGCGCCGAGTCGGCGCT | | U83C | 9 |
| cr282 | GTTTAAGAGCTAAGCTGGAAACAGCATAGCAAGTTTAAATAAGGCTAGT | 243 | G71C, | 1.04869375 |
| | CCGTTATCAACTTGAAAAAGTGCACCCGAGTCGGGTGT | | C72A, | 2 |
| | | | A73C, | |
| | | | U83G, | |
| | | | G84U, | |
| | | | C85G | |
| cr531 | GTTTAAGAGCTAAGCTGGTAACAGCATAGCAAGTTTAAATAAGGCTAGT | 244 | A18U | 1.04844440 |
| | CCGTTATCAACTTGAAAAAGTGGCACCGAGTCGGTGCT | | | 8 |
| cr814 | GTTTAAGAGCTAAGCTGGAAACAGCATAGCAAGTTTAAATAAGGCTAGT | 245 | C72G, | 1.04841612 |
| | CCGTTATCAACTTGAAAAAGTGGGTCCGAGTCGGACCT | | A73U, | 7 |
| | | | U83A, | |
| | | | G84C | |
| cr101 | GTTTAAGAGCTAAGCTGGAAACAGCATAGCAAGTTTAAATAAGGCTAGT | 246 | A73G | 1.04809498 |
| | CCGTTATCAACTTGAAAAAGTGGCGCCGAGTCGGTGCT | | | 2 |
| cr183 | GTTTAAGAGCTAAGCTGGAAACAGCATAGCAAGTTTAAATAAGGTTAAT | 247 | C44U, | 1.04725017 |
| | CCGTTATCAACTTGAGAAAGTGGCACCGAGTCGGTGCT | | G47A, | 3 |
| | | | A64G | |
| cr240 | GTTTAAGAGCTAAGCTGGAAACAGCATAGCAAGTTTAAATAAGGCTAGT | 248 | U55A | 1.04618338 |
| | CCGTTAACAACTTGAAAAAGTGGCACCGAGTCGGTGCT | | | 2 |
| cr171 | GTTTAAGAGCTAAGCTGGAAACAGCATAGCAAGTTTAAATAAGGCTAGT | 249 | U60C, | 1.04421806 |
| | CCGTTATCAACCTGAGAAGGTGGCACCGAGTCGGTGCT | | A64G, | 2 |
| | | | A67G | |
| cr809 | GTTTAAGAGCTAAGCTGGAAACAGCATAGCAAGTTTAAATAAGGCTAGT | 250 | U60G, | 1.04246214 |
| | CCGTTATCAACGTGAAAACGTGGAACCGAGTCGGTTCT | | A67C, | 6 |
| | | | C72A, | |
| | | | G84U | |
| cr356 | GTTTAAGAGCTAAGCTGGAAACAGCATAGCAAGTTTAAATAAGGCTAGT | 251 | A58C, | 1.04242407 |
| | CCGTTATCACCTTGAAAAAGGGACACCGAGTCGGTGTT | | U69G, | 7 |
| | | | G71A, | |
| | | | C85U | |
| cr687 | GTTTAAGAGCTAAGCTGGAAACAGCATAGCAAGTTTAAATAAGGCTAGT | 252 | C75U | 1.04175646 |
| | CCGTTATCAACTTGAAAAAGTGGCACTGAGTCGGTGCT | | | 8 |
| cr756 | GTTTAAGAGCTAAGCTGGAAACAGCATAGCAAGTTTAAATAAGGTTAAT | 253 | C44U, | 1.04120038 |
| | CCGTTATCAACTTGAAAAAGTGGCAACGAGTCGTTGCT | | G47A, | 3 |
| | | | C74A, | |
| | | | G82U | |
| cr623 | GTTTAAGAGCTAAGCTGGAAACAGCATAGCAAGTTTAAATAAGGCTAGT | 254 | A58C, | 1.04119280 |
| | CCGTTATCACTGTGAAAACAGGGCACCGAGTCGGTGCT | | C59U, | 2 |
| | | | U60G, | |
| | | | A67C, | |
| | | | G68A, | |
| | | | U69G | |
| cr685 | GTTTAAGAGCTAAGCTGGAAACAGCATAGCAAGTTTAAATAAGGCTAGT | 255 | A58G | 1.04107706 |
| | CCGTTATCAGCTTGAAAAAGTGGCACCGAGTCGGTGCT | | | |
| cr892 | GTTTAAGAGCTAAGCTGGAAACAGCATAGCAAGTTTAAATAAGGCTAGT | 256 | U60G, | 1.04106180 |
| | CCGTTATCAACGTGAAAACGTGGCGACGAGTCGTCGCT | | A67C, | 4 |
| | | | A73G, | |
| | | | C74A, | |
| | | | G82U, | |
| | | | U83C | |
| cr379 | GTTTAAGAGCTAAGCTGGAAACAGCCTAGCAAGTTTAAATAAGGCTAGT | 257 | A25C | 1.04104020 |
| | CCGTTATCAACTTGAAAAAGTGGCACCGAGTCGGTGCT | | | 2 |
| cr870 | GTTTAAGAGCTAAGCTGGAAACAGCAAAGCAAGTTTAAATAAGGCTAGT | 258 | U26A | 1.04045280 |
| | CCGTTATCAACTTGAAAAAGTGGCACCGAGTCGGTGCT | | | 5 |
| cr487 | GTTTAAGAGCTAAGCTGGAAACAGCATAGCACGTTTAAATAAGGCTAGT | 259 | A31C | 1.03900164 |
| | CCGTTATCAACTTGAAAAAGTGGCACCGAGTCGGTGCT | | | 3 |
| cr832 | GTTTAAGAGCTAAGCTGGAAACAGCATAGCAAGTTTAAATAAGGCTAGT | 260 | A58G, | 1.03887850 |
| | CCGTTATCAGCTTGAAAAAGCGGTGCCGAGTCGGCACT | | U69C, | 1 |
| | | | C72U, | |
| | | | A73G, | |
| | | | U83C, | |
| | | | G84A | |
| cr476 | GTTTAAGAGCTAAGCTGGAAACAGCATAGCAAGTTTAAATAAGGCTAGT | 261 | G71C, | 1.03871291 |
| | CCGTTATCAACTTGAAAAAGTGCGACCGAGTCGGTCGT | | C72G, | 4 |
| | | | G84C, | |
| | | | C85G | |
| cr691 | GTTTAAGAGCTAAGCTGGAAACAGCATAGCAAGTTTAAATAAGGCTAGT | 262 | C72G, | 1.03826758 |
| | CCGTTATCAACTTGAAAAAGTGGGCCCGAGTCGGGCCT | | A73C, | 3 |
| | | | U83G, | |
| | | | G84C | |
| cr821 | GTTTAAGAGCTAAGCTGGAAACAGCGTAGCAAGTTTAAATAAGGCTAGT | 263 | A25G | 1.03777650 |
| | CCGTTATCAACTTGAAAAAGTGGCACCGAGTCGGTGCT | | | 1 |
| cr727 | GTTTAAGAGCTAAGCTGGAAACAGCATAGCAAGTTTAAATAAGGCTAGT | 264 | G71U, | 1.03722526 |
| | CCGTTATCAACTTGAAAAAGTGTCGCCGAGTCGGCGAT | | A73G, | 3 |
| | | | U83C, | |
| | | | C85A | |
| cr483 | GTTTAAGAGCTAAGCTGGAAACAGCATAGCAAGTTTAAATAAGGCTAGT | 265 | U61A, | 1.03710417 |
| | CCGTTATCAACTAGAAATAGTGGCGTCGAGTCGACGCT | | A66U, | 5 |
| | | | A73G, | |
| | | | C74U, | |
| | | | G82A, | |
| | | | U83C | |
| cr776 | GTTTAAGAGCTAAGCTGGAAACAGCATAGCAAGTTTAAATAAGGCTAGT | 266 | A58U, | 1.03660795 |
| | CCGTTATCATCTAGAAATAGAGGCACCGAGTCGGTGCT | | U61A, | 2 |
| | | | A66U, | |
| | | | U69A | |
| cr335 | GTTTAAGAGCTAAGCTGGAAACAGCATAGCAAGTTTAAATAAGGCTAGT | 267 | G71A, | 1.03623132 |
| | CCGTTATCAACTTGAAAAAGTGACGCCGAGTCGGCGTT | | A73G, | 9 |
| | | | U83C, | |
| | | | C85U | |
| cr593 | GTTTAAGAGCTAAGCTGGAAACAGCATAGCAAGTTTAAATAAGGCTAGT | 268 | A58C, | 1.03547944 |
| | CCGTTATCACCTTGAAAAAGGGGCACCGAGTCGGTGCT | | U69G | 2 |
| cr616 | GTTTAAGAGCTAAGCTGGAAACAGCATAGCAAGTTTAAATAAGGCTAGT | 269 | C59G, | 1.03498308 |
| | CCGTTATCAAGATGAAAATCTGGCACCGAGTCGGTGCT | | U60A, | 7 |
| | | | A67U, | |
| | | | G68C | |
| cr320 | GTTTAAGAGCTAAGCTGGAAACAGCATAGCATGTTTAAATAAGGCTAGT | 270 | A31U, | 1.03491349 |
| | CCGTTATCAACTTGAGAAAGTGGCACCGAGTCGGTGCT | | A64G | 3 |
| cr410 | GTTTAAGAGCTAAGCTGGAAACAGCATAGCAAGTTTAAATAAGGCTAGT | 271 | A58G, | 1.03439489 |
| | CCGTTATCAGCTTGAGAAAGCGGCACCGAGTCGGTGCT | | A64G, | 2 |
| | | | U69C | |
| cr492 | GTTTAAGAGCTAAGCTGGAAACAGCATAGCAAGTTTAAATAAGGCTAGT | 272 | A54C | 1.03371058 |
| | CCGTTCTCAACTTGAAAAAGTGGCACCGAGTCGGTGCT | | | 1 |
| cr951 | GTTTAAGAGCTAAGCTGGAAACAGCATAGCAAGTTTAAATAAGGCTAGT | 273 | A64G, | 1.03356538 |
| | CCGTTATCAACTTGAGAAAGTGGCTCCGAGTCGGAGCT | | A73U, | 4 |
| | | | U83A | |
| cr964 | GTTTAAGAGCTAAGCTGGAAACAGCATAGCTAGTTTAAATAAGGCTAGT | 274 | A30U, | 1.03345180 |
| | CCGTTATCAACTTGAGAAAGTGGCACCGAGTCGGTGCT | | A64G | 7 |
| cr263 | GTTTAAGAGCTAAGCTGGAAACAGCATAGCAAGTTTAAATAAGGCTAGT | 275 | A65C | 1.03337417 |
| | CCGTTATCAACTTGAACAAGTGGCACCGAGTCGGTGCT | | | 4 |
| cr214 | GTTTAAGAGCTAAGCTGGAAACAGCATAGCAAGTTTAAATAAGGCTAGT | 276 | A64G, | 1.03321224 |
| | CCGTTATCAACTTGAGAAAGTGGCAACGAGTCGTTGCT | | C74A, | 2 |
| | | | G82U | |
| cr628 | GTTTAAGAGCTAAGCTGGAAACAGCATAGCAAGTTTAAATAAGGCTAGT | 277 | C72U, | 1.03313967 |
| | CCGTTATCAACTTGAAAAAGTGGTACCAAGTTGGTACT | | G76A, | 6 |
| | | | C80U, | |
| | | | G84A | |
| cr704 | GTTTAAGAGCTAAGCTGGAAACAGCATAGCAAGTTTAAATAAGGCTAGT | 278 | U69C | 1.03273911 |
| | CCGTTATCAACTTGAAAAAGCGGCACCGAGTCGGTGCT | | | |
| cr524 | GTTTAAGAGCTAAGCTGGAAACAGCATAGCAAGTTTAAATAAGGCTAGT | 279 | A63U | 1.03249928 |
| | CCGTTATCAACTTGTAAAAGTGGCACCGAGTCGGTGCT | | | |
| cr054 | GTTTAACCGCTAAGCTGGAAACAGCATAGCAAGTTTAAATAAGGCTAGT | 280 | G6C, | 1.03244356 |
| | CCGTTATCAACTTGAAAAAGTGGCACCGAGTCGGTGCT | | A7C | 5 |
| cr455 | GTTTAAGAGCTAAGCTGGAAACAGCATAGCAAGTTTAAATAAGGCTAGT | 281 | C59U, | 1.03107200 |
| | CCGTTATCAATTGGAAACAATGGCACCGAGTCGGTGCT | | U61G, | 1 |
| | | | A66C, | |
| | | | G68A | |
| cr352 | GTTTAAGAGCTAAGCTGGAAACAGCATAGCAAGTTTAAATAAGGCTAGT | 282 | A58C | 1.03104846 |
| | CCGTTATCACCTTGAAAAAGTGGCACCGAGTCGGTGCT | | | 1 |
| cr902 | GTTTAAGAGCTAAGCTGGAAACAGCATAGCAAGTTTAAATAAGGCTAGT | 283 | G71A, | 1.0308018 |
| | CCGTTATCAACTTGAAAAAGTGACACCGAGTCGGTGTT | | C85U | |
| cr109 | GTTTAAGAGCTAAGCTGGAAACAGCATAGCAAGTTTAAATAAGGCTAGT | 284 | U60A, | 1.03065158 |
| | CCGTTATCAACAAGAAATTGTGGCACCGAGTCGGTGCT | | U61A, | 4 |
| | | | A66U, | |
| | | | A67U | |
| cr070 | GTTTAACGGCTAAGCTGGAAACAGCATAGCAAGTTTAAATAAGGCTAGT | 285 | G6C, | 1.02998344 |
| | CCGTTATCAACTTGAAAAAGTGGCACCGAGTCGGTGCT | | A7G | 2 |
| cr271 | GTTTAAGAGCTAAGCTGGAAACAGCATAGCAAGTTTAAATAAGGCTAGT | 286 | C72U, | 1.02969848 |
| | CCGTTATCAACTTGAAAAAGTGGTCCCGAGTCGGGACT | | A73C, | 2 |
| | | | U83G, | |
| | | | G84A | |
| cr129 | GTTTAAGAGCTAAGCTGGAAACAGCATAGCAAGTTTAAATAAGGCTAGT | 287 | U60G, | 1.02876213 |
| | CCGTTATCAACGTGAGAACGTGGCACCGAGTCGGTGCT | | A64G, | 2 |
| | | | A67C | |
| cr497 | GTTTAAGAGCTAAGCTGGAAACAGCATAGCAAGTTTAAATAAGGCTAGT | 288 | A64G, | 1.02842968 |
| | CCGTTATCAACTTGAGAAAGTGGAACCGAGTCGGTTCT | | C72A, | 3 |
| | | | G84U | |
| cr828 | GTTTAAGAGCTAAGCTGGAAACAGCATAGCAGGTTTAAATAAGGCTAGT | 289 | A31G | 1.02830507 |
| | CCGTTATCAACTTGAAAAAGTGGCACCGAGTCGGTGCT | | | 5 |
| cr235 | GTTTAAGAGCTAAGCTGGAAACAGCATAGCAAGTTTAAATAAGGCTAGT | 290 | U60A, | 1.02736287 |
| | CCGTTATCAACATGAGAATGTGGCACCGAGTCGGTGCT | | A64G, | 9 |
| | | | A67U | |
| cr882 | GTTTAAGAGCTAAGCTGGAAACAGCATAGCAAGTTTAAATAAGGCTAGT | 291 | G62C | 1.02729278 |
| | CCGTTATCAACTTCAAAAAGTGGCACCGAGTCGGTGCT | | | 6 |
| cr515 | GTTTAAGAGCTAAGCTGTAAACAGCATAGCAAGTTTAAATAAGGCTAGT | 292 | G17U | 1.02718842 |
| | CCGTTATCAACTTGAAAAAGTGGCACCGAGTCGGTGCT | | | 6 |
| cr434 | GTTTAAGAGCTAAGCTGGAAACAGCATAGCAAGTTTAAATAAGGCTAGT | 293 | G62C, | 1.02709347 |
| | CCGTTATCAACTTCATAAAGTGGCACCGAGTCGGTGCT | | A64U | 9 |
| cr797 | GTTTAAGAGCTAAGCTGGAAACAGCATAGCAAGTTTAAATAAGGCTAGT | 294 | U53G | 1.02698062 |
| | CCGTGATCAACTTGAAAAAGTGGCACCGAGTCGGTGCT | | | 9 |
| cr884 | GTTTAAGAGCTAAGCTGGAAACAGCATAGCAAGTTTAAATAAGGCTAGT | 295 | A58C, | 1.02683106 |
| | CCGTTATCACCTAGAAATAGGGTCACCGAGTCGGTGAT | | U61A, | 1 |
| | | | A66U, | |
| | | | U69G, | |
| | | | G71U, | |
| | | | C85A | |
| cr610 | GTTTAAGAGCTAAGCTGGAAACAGCATAGCAAGTTTAAATAAGGCTAGT | 296 | C59G, | 1.02674677 |
| | CCGTTATCAAGATGAAAATCTGACACCGAGTCGGTGTT | | U60A, | 7 |
| | | | A67U, | |
| | | | G68C, | |
| | | | G71A, | |
| | | | C85U | |
| cr118 | GTTTAAGAGCTAAGCTGGAAACGGCATAGCAAGTTTAAATAAGGCTAGT | 297 | A22G | 1.02533901 |
| | CCGTTATCAACTTGAAAAAGTGGCACCGAGTCGGTGCT | | | 5 |
| cr412 | GTTTAAGAGCTAAGCTGGAAACAGCATAGCAAGTTTAAATAAGGCTAGT | 298 | C59G, | 1.02512486 |
| | CCGTTATCAAGTTGAAAAACTGGCACCGAGTCGGTGCT | | G68C | |
| cr929 | GTTTAAGAGCTAAGCTGGAAACAGCATAGCAAGTTTAAATAAGGCTAGT | 299 | G71U | 1.02508935 |
| | CCGTTATCAACTTGAAAAAGTGTCACCGAGTCGGTGCT | | | 5 |
| cr858 | GTTTAAGAGCTAAGCTGGAGACAGCATAGCAAGTTTAAATAAGGCTAGT | 300 | A19G | 1.02503584 |
| | CCGTTATCAACTTGAAAAAGTGGCACCGAGTCGGTGCT | | | |
| cr896 | GTTTAAGAGCTAAGCTGGAAACAGCATAGCAAGTTTAAATAAGGCTAGT | 301 | C72A, | 1.02474392 |
| | CCGTTATCAACTTGAAAAAGTGGACCCGAGTCGGGTCT | | A73C, | 2 |
| | | | U83G, | |
| | | | G84U | |
| cr334 | GTTTAAGAGCTAAGCTGGAAACAGCATAGCAAGTTTAAATAAGGCTAGT | 302 | C59G, | 1.02464779 |
| | CCGTTATCAAGTGGAAACACTGGCACCAAGTTGGTGCT | | U61G, | 9 |
| | | | A66C, | |
| | | | G68C, | |
| | | | G76A, | |
| | | | C80U | |
| cr934 | GTTTAAGAGCTAAGCTGGAAACAGCATAGCAAGTTTAAATAAGGCTTGT | 303 | A46U, | 1.02413232 |
| | CCGTTACCAACTTGAAAAAGTGGCACCGATTCGGTGCT | | U55C, | 6 |
| | | | G78U | |
| cr444 | GTTTAAGAGCTAAGCTGGAAACAGCATAGCAAGTTTAAATAAGGCTAGT | 304 | A64C | 1.02269899 |
| | CCGTTATCAACTTGACAAAGTGGCACCGAGTCGGTGCT | | | 7 |
| cr140 | GTTTAAGAGCTAAGCTGGAAACAGCATAGCAAGTTTAAATAAGGCTAGT | 305 | C59U, | 1.02259341 |
| | CCGTTATCAATCTGAAAAGATGGCACCGAGTCGGTGCT | | U60C, | 3 |
| | | | A67G, | |
| | | | G68A | |
| cr600 | GTTTAAGAGCTAAGCTGGAAACAGCATAGCAAGTTTAAATAAGGCTAGT | 306 | C59G, | 1.02124331 |
| | CCGTTATCAAGTTGAGAAACTGGCACCGAGTCGGTGCT | | A64G, | 6 |
| | | | G68C | |
| cr710 | GTTTAAGAGCTAAGCTGGAAACAGCATAGCAAGTTTAAATAAGGCTAGT | 307 | C74G, | 1.02097870 |
| | CCGTTATCAACTTGAAAAAGTGGCAGCGAGTCGCTGCT | | G82C | 3 |
| cr345 | GTTTAAGAGCTAAGCTGGAACCAGCATAGCAAGTTTAAATAAGGCTAGT | 308 | A20C | 1.02071344 |
| | CCGTTATCAACTTGAAAAAGTGGCACCGAGTCGGTGCT | | | 4 |
| cr978 | GTTTAAGAGCTAAGCTGGAAACAGCATAGCAAGTTTAAATAAGGCTAGT | 309 | C72A, | 1.02058245 |
| | CCGTTATCAACTTGAAAAAGTGGAAACGAGTCGTTTCT | | C74A, | 8 |
| | | | G82U, | |
| | | | G84U | |
| cr561 | GTTTAAGAGCTAAGCTGGAAACAGCATAGCAAGTTTAAATAAGGCTTGT | 310 | A46U | 1.02057043 |
| | CCGTTATCAACTTGAAAAAGTGGCACCGAGTCGGTGCT | | | 7 |
| cr801 | GTTTAAGAGCTAAGCTGGAAACAGCATAGCAAGTTTAAATAAGGCTAGT | 311 | A58G, | 1.02042822 |
| | CCGTTATCAGCTTGAAAAAGCGGAAGCGAGTCGCTTCT | | U69C, | 2 |
| | | | C72A, | |
| | | | C74G, | |
| | | | G82C, | |
| | | | G84U | |
| cr948 | GTTTAAGAGCTAAGCTGGAAACAGCATAGCAAGTTTAAATAAGGCTAGT | 312 | A66G | 1.02039913 |
| | CCGTTATCAACTTGAAAGAGTGGCACCGAGTCGGTGCT | | | |
| cr888 | GTTTAAGAGCTAAGCTGGAAACAGCATAGCAAGTTTAAATAAGGCTAGT | 313 | G62U, | 1.02030698 |
| | CCGTTATCAACTTTACAAAGTGGCACCGAGTCGGTGCT | | A64C | 5 |
| cr020 | GTTTAATAGCTAAGCTGGAAACAGCATAGCAAGTTTAAATAAGGCTAGT | 314 | G6U | 1.02026526 |
| | CCGTTATCAACTTGAAAAAGTGGCACCGAGTCGGTGCT | | | 4 |
| cr323 | GTTTAAGAGCTAAGCTGGAAACAGCATAGCAGGTTTAAATAAGGCTAGT | 315 | A31G, | 1.02021061 |
| | CCGTTATCAACTTGACAAAGTGGCACCGAGTCGGTGCT | | A64C | 3 |
| cr019 | GTTTAACAGCTAAGCTGGAAACAGCATAGCAAGTTTAAATAAGGCTAGT | 316 | G6C | 1.01913954 |
| | CCGTTATCAACTTGAAAAAGTGGCACCGAGTCGGTGCT | | | |
| cr408 | GTTTAAGAGCTAAGCTGGAAACAGCATAGCAAGTTTAAATAAGGCTAGT | 317 | A64G, | 1.0182334 |
| | CCGTTATCAACTTGAGAAAGTGACACCGAGTCGGTGTT | | G71A, | |
| | | | C85U | |
| cr730 | GTTTAAGAGCTAAGCTGGAAACAGCATAGCAAGTTTAAATAAGGCTAGT | 318 | C72G, | 1.01798666 |
| | CCGTTATCAACTTGAAAAAGTGGGGGCGAGTCGCCCCT | | A73G, | 1 |
| | | | C74G, | |
| | | | G82C, | |
| | | | U83C, | |
| | | | G84C | |
| cr603 | GTTTAAGAGCTAAGCTGGAAACAGCATAGCAAGTTTAAATAAGGCTAGT | 319 | A64G, | 1.01766316 |
| | CCGTTATCAACTTGAGCAAGTGGCACCGAGTCGGTGCT | | A65C | 5 |
| cr557 | GTTTAAGAGCTAAGCTGGCAACAGCATAGCAAGTTTAAATAAGGCTAGT | 320 | A18C | 1.01765734 |
| | CCGTTATCAACTTGAAAAAGTGGCACCGAGTCGGTGCT | | | 7 |
| cr283 | GTTTAAGAGCTAAGCTGGAAACAGCATAGCAAGTTTAAATAAGGCTAGT | 321 | G71A | 1 .01760152 |
| | CCGTTATCAACTTGAAAAAGTGACACCGAGTCGGTGCT | | | 6 |
| cr464 | GTTTAAGAGCTAAGCTGGAAACAGCATAGCAAGTTTAAATAAGGCTAGT | 322 | A58C, | 1.01749147 |
| | CCGTTATCACCTTGAAAAAGGGGCACCAAGTTGGTGCT | | U69G, | 7 |
| | | | G76A, | |
| | | | C80U | |
| cr592 | GTTTAAGAGCTAAGCTGGAAACAGCATAGCAAGTTTAAATAAGGTTAAT | 323 | C44U, | 1.0173258 |
| | CCGTTATCAACTTGAAAAAGTGGCACCGAGTCGGTGCT | | G47A | |
| cr971 | GTTTAAGAGCTAAGCTGGAAACAGCATAGCAAGTTTAAATAAGGCTAGT | 324 | G62A, | 1.01727269 |
| | CCGTTATCAACTTAATAAAGTGGCACCGAGTCGGTGCT | | A64U | 6 |
| cr366 | GTTTAAGAGCTAAGCTGGAAACAGCATAGCAAGTTTAAATAAGGCTAGT | 325 | A58G, | 1.01699031 |
| | CCGTTATCAGCTTGAAAAAGCGGCACTGAGTCAGTGCT | | U69C, | 9 |
| | | | C75U, | |
| | | | G81A | |
| cr018 | GTTTAAAAGCTAAGCTGGAAACAGCATAGCAAGTTTAAATAAGGCTAGT | 326 | G6A | 1.01664409 |
| | CCGTTATCAACTTGAAAAAGTGGCACCGAGTCGGTGCT | | | 9 |
| cr701 | GTTTAAGAGCTAAGCTGGAAACAGCATAGCAAGTTTAAATAAGGCTAGT | 327 | G78C | 1.01640096 |
| | CCGTTATCAACTTGAAAAAGTGGCACCGACTCGGTGCT | | | 8 |
| cr354 | GTTTAAGAGCTAAGCTGGAAACAGCATAGCAAGTTTAAATAAGGCTAGT | 328 | A58G, | 1.01627913 |
| | CCGTTATCAGCTTGAAAAAGCGATACCGAGTCGGTATT | | U69C, | 7 |
| | | | G71A, | |
| | | | C72U, | |
| | | | G84A, | |
| | | | C85U | |
| cr494 | GTTTAAGAGCTAAGCTGGAAACAGCATAGCAAGTTTAAATAAGGCTAGT | 329 | A66U | 1.01625224 |
| | CCGTTATCAACTTGAAATAGTGGCACCGAGTCGGTGCT | | | 6 |
| cr302 | GTTTAAGAGCTAAGCTGGAAACAGCATAGCAAGTTTAAATAAGGCTAGT | 330 | U60G, | 1.01620407 |
| | CCGTTATCAACGTGAAAACGTGGTCCCGAGTCGGGACT | | A67C, | 2 |
| | | | C72U, | |
| | | | A73C, | |
| | | | U83G, | |
| | | | G84A | |
| cr113 | GTTTAAGAGCTAAGCTGGAAACAGCATAGCAAGTTTAAATAAGGCTAGT | 331 | A54U | 1.01605369 |
| | CCGTTTTCAACTTGAAAAAGTGGCACCGAGTCGGTGCT | | | 7 |
| cr941 | GTTTAAGAGCTAAGCTGGAAACAGCATAGCAAGTTTAAATAAGGCTAGT | 332 | C59A, | 1.01572195 |
| | CCGTTATCAAATGGAAACATTGACACCGAGTCGGTGTT | | U61G, | 9 |
| | | | A66C, | |
| | | | G68U, | |
| | | | G71A, | |
| | | | C85U | |
| cr655 | GTTTAAGAGCTAAGCTGGAAACAGCATAGCAAGTTTAAATAAGGCTAGT | 333 | U53C | 1.01557092 |
| | CCGTCATCAACTTGAAAAAGTGGCACCGAGTCGGTGCT | | | 8 |
| cr619 | GTTTAAGAGCTAAGCTGGAAACAGCATAGCAAGTTTAAATAAGGCTAGT | 334 | A63G | 1.01473992 |
| | CCGTTATCAACTTGGAAAAGTGGCACCGAGTCGGTGCT | | | 7 |
| cr121 | GTTTAAGAGCTAAGCTGGAAACAGCATAGCAAGTTTAAATAAGGCTAGT | 335 | A58U, | 1.01388795 |
| | CCGTTATCATCATGAAAATGAGGCACCGAGTCGGTGCT | | U60A, | |
| | | | A67U, | |
| | | | U69A | |
| cr898 | GTTTAAGAGCTAAGCTGGAAACAGCATAGCACGTTTAAATAAGGCTAGT | 336 | A31C, | 1.01381218 |
| | CCGTTATCAACTTGAGAAAGTGGCACCGAGTCGGTGCT | | A64G | 5 |
| cr239 | GTTTAAGAGCTAAGCTGGAAACAGCATAGCAAGTTTAAATAAGGCTAGT | 337 | U52C, | 1.01366903 |
| | CCGCTATCAACTTGTAAAAGTGGCACCGAGTCGGTGCT | | A63U | 5 |
| cr980 | GTTTAAGAGCTAAGCTGGAAACAGCATAGCAAGTTTAAATAAGGCTAGT | 338 | U52C | 1.01366157 |
| | CCGCTATCAACTTGAAAAAGTGGCACCGAGTCGGTGCT | | | 3 |
| cr428 | GTTTAAGAGCTAAGCTGGAAACAGCATAGCAAGTTTAAATAAGGCTAGT | 339 | A63C, | 1.01283479 |
| | CCGTTATCAACTTGCATAAGTGGCACCGAGTCGGTGCT | | A65U | 7 |
| cr433 | GTTTAAGAGCTAAGCTGGAAACAGCATAGCAAGTTTAAATAAGGCTAGT | 340 | A65U | 1.01239334 |
| | CCGTTATCAACTTGAATAAGTGGCACCGAGTCGGTGCT | | | 7 |
| cr377 | GTTTAAGAGCTAAGCTGGAAACAGCATAGCAAGTTTAAATAAGGCTAGT | 341 | U55G | 1.01216392 |
| | CCGTTAGCAACTTGAAAAAGTGGCACCGAGTCGGTGCT | | | 5 |
| cr423 | GTTTAAGAGCTAAGCTGGAAACAGCATAGCAAGTTTAAATAAGGCTAGT | 342 | A58G, | 1.01174760 |
| | CCGTTATCAGCTTGAAAAAGCGGTACCGAGTCGGTACT | | U69C, | 6 |
| | | | C72U, | |
| | | | G84A | |
| cr690 | GTTTAAGAGCTAAGCTGGAAACAGCAGAGCAAGTTTAAATAAGGCTAGT | 343 | U26G | 1.01099309 |
| | CCGTTATCAACTTGAAAAAGTGGCACCGAGTCGGTGCT | | | 2 |
| cr037 | GTTTAATCGCTAAGCTGGAAACAGCATAGCAAGTTTAAATAAGGCTAGT | 344 | G6U, | 1.01037945 |
| | CCGTTATCAACTTGAAAAAGTGGCACCGAGTCGGTGCT | | A7C | 9 |
| cr642 | GTTTAAGAGCTAAGCTGGAAACAGCATAGCATGTTTAAATAAGGCTAGT | 345 | A31U | 1.00836434 |
| | CCGTTATCAACTTGAAAAAGTGGCACCGAGTCGGTGCT | | | 8 |
| cr715 | GTTTAAGAGCTAAGCTGGAAACAGCATAGCGAGTTTAAATAAGGCTAGT | 346 | A30G | 1.00799007 |
| | CCGTTATCAACTTGAAAAAGTGGCACCGAGTCGGTGCT | | | 9 |
| cr632 | GTTTAAGAGCTAAGCTGGAAACAGCATAGCAAGTTTAAATAAGGCTAGT | 347 | A63C, | 1.00755206 |
| | CCGTTATCAACTTGCAGAAGTGGCACCGAGTCGGTGCT | | A65G | 1 |
| cr348 | GTTTAAGAGCTAAGCTGGAAGCAGCATAGCAAGTTTAAATAAGGCTAGT | 348 | A20G | 1.00755146 |
| | CCGTTATCAACTTGAAAAAGTGGCACCGAGTCGGTGCT | | | |
| cr510 | GTTTAAGAGCTAAGCTGGAAACAGCATAGCAAGTTTAAATAAGGCTAGT | 349 | A58U, | 1.00746555 |
| | CCGTTATCATGTTGAAAAACAGGCACCGAGTCGGTGCT | | C59G, | 1 |
| | | | G68C, | |
| | | | U69A | |
| cr771 | GTTTAAGAGCTAAGCTGGAAACAGCATAGCAAGTTTAAATAAGGCTAGT | 350 | A64G, | 1.00740692 |
| | CCGTTATCAACTTGAGAAAGTGCCACCGAGTCGGTGGT | | G71C, | 7 |
| | | | C85G | |
| cr606 | GTTTAAGAGCTAAGCTGGAAACAGCATAGCAAGTTTAAATAAGGCTAGT | 351 | C59G, | 1.00738312 |
| | CCGTTATCAAGTTGAAAAACTGGCCTCGAGTCGAGGCT | | G68C, | 9 |
| | | | A73C, | |
| | | | C74U, | |
| | | | G82A, | |
| | | | U83G | |
| cr144 | GTTTAAGAGCTAAGCTGGAAACAGCATAGCAAGTTTAAATAAGGCTAGT | 352 | A58U, | 1.00728202 |
| | CCGTTATCATGTAGAAATACAGGCACCGAGTCGGTGCT | | C59G, | 5 |
| | | | U61A, | |
| | | | A66U, | |
| | | | G68C, | |
| | | | U69A | |
| cr559 | GTTTAAGAGCTAAGCTGGAAACAGCATAGCAAGTTTAAATAAGGCTAGT | 353 | C72U, | 1.00721348 |
| | CCGTTATCAACTTGAAAAAGTGGTACCGAGTCGGTACT | | G84A | 5 |
| cr365 | GTTTAAGAGCTAAGCTGGAAACAGCATAGCAAGTTTAAATAAGGCTAGT | 354 | U53A | 1.00681907 |
| | CCGTAATCAACTTGAAAAAGTGGCACCGAGTCGGTGCT | | | 6 |
| cr232 | GTTTAAGAGCTAAGCTGGAAACAGCACAGCAAGTTTAAATAAGGCTAGT | 355 | U26C | 1.00634227 |
| | CCGTTATCAACTTGAAAAAGTGGCACCGAGTCGGTGCT | | | 3 |
| cr139 | GTTTAAGAGCTAAGCTGGAAACAGCATAGCAAGTTTAAATAAGGCTAGT | 356 | C74A, | 1.00580446 |
| | CCGTTATCAACTTGAAAAAGTGGCAACGAGTCGTTGCT | | G82U | 5 |
| cr672 | GTTTAAGAGCTAAGCTGGAAACAGCATAGCAAGTTTAAATAAGGCTAGT | 357 | C74U | 1.00454783 |
| | CCGTTATCAACTTGAAAAAGTGGCATCGAGTCGGTGCT | | | 9 |
| cr656 | GTTTAAGAGCTAAGCTGGAAACAGCATAGCAAGTTTAAATAAGGCTAGT | 358 | A73C, | 1.00440038 |
| | CCGTTATCAACTTGAAAAAGTGGCCCCGAGTCGGGGCT | | U83G | 3 |
| cr393 | GTTTAAGAGCTAAGCTGGAAACAGCATAGCAAGTTTAAATAAGGCTAGT | 359 | A64C, | 1.00402537 |
| | CCGTTATCAACTTGACAAAGTGGCACCGAATCGGTGCT | | G78A | 5 |
| cr968 | GTTTAAGAGCTAAGCTGGAAACAGCATAGCAAGTTTAAATAAGGCTAGT | 360 | C72A, | 1.00367609 |
| | CCGTTATCAACTTGAAAAAGTGGAACCGAGTCGGTTCT | | G84U | 2 |
| cr155 | GTTTAAGAGCTAAGCTGGAAACAGCATAGCAAGTTTAAATAAGGCTAGT | 361 | C59U, | 1.00316492 |
| | CCGTTATCAATTGGAAACAATGGCATCGAGTCGATGCT | | U61G, | 2 |
| | | | A66C, | |
| | | | G68A, | |
| | | | C74U, | |
| | | | G82A | |
| cr901 | GTTTAAGAGCTAAGCTGGAAACAGCATAGCAAGTTTAAATAAGGCTAGT | 362 | A65G | 1.00302100 |
| | CCGTTATCAACTTGAAGAAGTGGCACCGAGTCGGTGCT | | | 4 |
| cr945 | GTTTAAGAGCTAAGCTGGAAACAGCATAGCAAGTTTAAATAAGGCTAGT | 363 | A65G, | 1.00236082 |
| | CCGTTATCAACTTGAAGAAGTGGCACCGATTCGGTGCT | | G78U | |
| cr128 | GTTTAAGAGCTAAGCTGGAAACAGCATAGCAAGTTTAAATAAGGCTAGT | 364 | A58G, | 1.00194867 |
| | CCGTTATCAGCTTGAAAAAGCGGCACAGAGTCTGTGCT | | U69C, | 8 |
| | | | C75A, | |
| | | | G81U | |
| cr851 | GTTTAAGAGCTAAGCTGGAAACAGCATAGCAAGTTTAAATAAGGCTAGT | 365 | A58G, | 1.00191002 |
| | CCGTTATCAGCTTGAAAAAGCGGCACCAAGTTGGTGCT | | U69C, | |
| | | | G76A, | |
| | | | C80U | |
| cr923 | GTTTAAGAGCTAAGCTGGAAACAGCATAGCAAGTTTAAATAAGGCTAGT | 366 | G71A, | 1.00154036 |
| | CCGTTATCAACTTGAAAAAGTGACCCCGAGTCGGGGTT | | A73C, | 9 |
| | | | U83G, | |
| | | | C85U | |
| cr722 | GTTTAAGAGCTAAGCTGGAAACAGCATAGCAAGTTTAAATAAGGCTAGT | 367 | A64G, | 1.00111557 |
| | CCGTTATCAACTTGAGAAAGTGGCCCCGAGTCGGGGCT | | A73C, | 9 |
| | | | U83G | |
| cr995 | GTTTAAGAGCTAAGCTGGAAACAGCATAGCAAGTTTAAATAAGGCTAGT | 368 | | 1 |
| | CCGTTATCAACTTGAAAAAGTGGCACCGAGTCGGTGCT | | | |
| cr392 | GTTTAAGAGCTAAGCTGGAAACAGCATAGCATGTTTAAATAAGGCTAGT | 369 | A31U, | 0.99994239 |
| | CCGTTATCAACTTGATAAAGTGGCACCGAGTCGGTGCT | | A64U | 9 |
| cr947 | GTTTAAGAGCTAAGCTGGGAACAGCATAGCAAGTTTAAATAAGGCTAGT | 370 | A18G | 0.99972128 |
| | CCGTTATCAACTTGAAAAAGTGGCACCGAGTCGGTGCT | | | 2 |
| cr172 | GTTTAAGAGCTAAGCTGGAAACAGCATAGCAAGTTTAAATAAGGCTTGT | 371 | A46U, | 0.99962027 |
| | CCGTTATCAACTTGTAAAAGTGGCACCGAGTCGGTGCT | | A63U | 6 |
| cr489 | GTTTAAGAGCTAAGCTGGAAACAGCATAGCAAGTTTAAATAAGGCTAGT | 372 | A64G, | 0.99912564 |
| | CCGTTATCAACTTGAGAAAGTGGGACCGAGTCGGTCCT | | C72G, | 1 |
| | | | G84C | |
| cr195 | GTTTAAGAGCTAAGCTGGAAACAGCATAGCAAGTTTAAATAAGGCTAGT | 373 | A63C, | 0.99859821 |
| | CCGTTATCAACTTGCAAAAGTGGCACCGATTCGGTGCT | | G78U | 3 |
| cr956 | GTTTAAGAGCTAAGCTGGAAACAGCATAGCAAGTTTAAATAAGGCAAGT | 374 | U45A | 0.99826701 |
| | CCGTTATCAACTTGAAAAAGTGGCACCGAGTCGGTGCT | | | 4 |
| cr269 | GTTTAAGAGCTAAGCTGGAAACAGCATAGCAAGTTTAAATAAGGCTAGT | 375 | G71C, | 0.99791063 |
| | CCGTTATCAACTTGAAAAAGTGCAACCGAGTCGGTTGT | | C72A, | 1 |
| | | | G84U, | |
| | | | C85G | |
| cr713 | GTTTAAGAGCTAAGCTGGAAACAGCATAGCAAGTTTAAATAAGGCTAGT | 376 | U61A, | 0.99789243 |
| | CCGTTATCAACTAGAGATAGTGGCACCGAGTCGGTGCT | | A64G, | 3 |
| | | | A66U | |
| cr152 | GTTTAAGAGCTAAGCTGGAAACAGCATAGCAAGTTTAAATAAGGCTAGT | 377 | G71A, | 0.99708807 |
| | CCGTTATCAACTTGAAAAAGTGAAACCGAGTCGGTTTT | | C72A, | 7 |
| | | | G84U, | |
| | | | C85U | |
| cr774 | GTTTAAGAGCTAAGCTGGACACAGCATAGCAAGTTTAAATAAGGCTAGT | 378 | A19C | 0.99676639 |
| | CCGTTATCAACTTGAAAAAGTGGCACCGAGTCGGTGCT | | | 6 |
| cr666 | GTTTAAGAGCTAAGCTGGAAACAGCATAGCAAGTTTAAATAAGGTTAAT | 379 | C44U, | 0.99598525 |
| | CCGTTATCAACTTGAAAAAGTGGAGCCGAGTCGGCTCT | | G47A, | 1 |
| | | | C72A, | |
| | | | A73G, | |
| | | | U83C, | |
| | | | G84U | |
| cr698 | GTTTAAGAGCTAAGCTGGAAACAGCATAACAAGTTTAAATAAGGCTAGT | 380 | G28A, | 0.99559758 |
| | CCGTAATCAACTTGTAAAAGTGGCACCGAGTCGGTGCT | | U53A, | 4 |
| | | | A63U | |
| cr789 | GTTTAAGAGCTAAGCTGGAAACAGCATAGCAAGTTTAAATAAGGCTAGT | 381 | G71C, | 0.99547256 |
| | CCGTTATCAACTTGAAAAAGTGCCACCGAGTCGGTGGT | | C85G | 7 |
| cr932 | GTTTAAGAGCTAAGCTGGAAACAGCATAGTAAGTTTAAATAAGGCTAGT | 382 | C29U | 0.99466604 |
| | CCGTTATCAACTTGAAAAAGTGGCACCGAGTCGGTGCT | | | 4 |
| cr893 | GTTTAAGAGCTAAGCTGGAAACAGCATAGCAAGTTTAAATAAGGCTAGT | 383 | C85U | 0.99323133 |
| | CCGTTATCAACTTGAAAAAGTGGCACCGAGTCGGTGTT | | | 9 |
| cr446 | GTTTAAGAGCTAAGCTGGAAACAGCATAGCAAGTTTAAATAAGGTTAAT | 384 | C44U, | 0.99264076 |
| | CCGTTATCATCTTGAAAAAGAGGGACCGAGTCGGTCCT | | G47A, | 1 |
| | | | A58U, | |
| | | | U69A, | |
| | | | C72G, | |
| | | | G84C | |
| cr145 | GTTTAAGAGCTAAGCTGGAAACAGCATAGCAAGTTTAAATAAGGCTAGT | 385 | A64U | 0.99249969 |
| | CCGTTATCAACTTGATAAAGTGGCACCGAGTCGGTGCT | | | 2 |
| cr636 | GTTTAAGAGCTAAGCTGGAAACAGCATAGCAAGTTTAAATAAGGCTAGT | 386 | A63G, | 0.99198315 |
| | CCGTTATCAACTTGGTAAAGTGGCACCGAGTCGGTGCT | | A64U | 4 |
| cr839 | GTTTAAGAGCTAAGCTGGAAACAGCATAGCAAGTTTAAATAAGGCTAGT | 387 | A64G | 0.99180202 |
| | CCGTTATCAACTTGAGAAAGTGGCACCGAGTCGGTGCT | | | 3 |
| cr023 | GTTTAAGGGCTAAGCTGGAAACAGCATAGCAAGTTTAAATAAGGCTAGT | 388 | A7G | 0.99173308 |
| | CCGTTATCAACTTGAAAAAGTGGCACCGAGTCGGTGCT | | | |
| cr604 | GTTTAAGAGCTAAGCTGGAAACAGCATAGCAAGTTTAAATAAGGCAAGT | 389 | U45A, | 0.99123350 |
| | CCGTTATCAACTTGACAAAGTGGCACCGAGTCGGTGCT | | A64C | 4 |
| cr653 | GTTTAAGAGCTAAGCTGGAAACAGCATAGCAAGTTTAAATAAGGCTAGT | 390 | C75A, | 0.99069367 |
| | CCGTTATCAACTTGAAAAAGTGGCACAGAGTCTGTGCT | | G81U | 2 |
| cr321 | GTTTAAGAGCTAAGCTGGAAACAGCATAGCCAGTTTAAATAAGGCTAGT | 391 | A30C | 0.99062396 |
| | CCGTTATCAACTTGAAAAAGTGGCACCGAGTCGGTGCT | | | 1 |
| cr670 | GTTTAAGAGCTAAGCTGGAAACAGCATAGCAAGTTTAAATAAGGCTAGT | 392 | U69G | 0.99028041 |
| | CCGTTATCAACTTGAAAAAGGGGCACCGAGTCGGTGCT | | | 1 |
| cr478 | GTTTAAGAGCTAAGCTGGAAACAGCATGGCAAGTTTAAATAAGGCTAGT | 393 | A27G | 0.98981927 |
| | CCGTTATCAACTTGAAAAAGTGGCACCGAGTCGGTGCT | | | 5 |
| cr609 | GTTTAAGAGCTAAGCTGGAAACAGCATAGCATGTTTAAATAAGGCTAGT | 394 | A31U, | 0.98947882 |
| | CCGTTATCAACTTAAAAAAGTGGCACCGAATCGGTGCT | | G62A, | 6 |
| | | | G78A | |
| cr353 | GTTTAAGAGCTAAGCTGGAAACAGCATAGCAAGTTTAAATAAGGCTAGT | 395 | U61A | 0.98845143 |
| | CCGTTATCAACTAGAAAAAGTGGCACCGAGTCGGTGCT | | | 2 |
| cr669 | GTTTAAGAGCTAAGCTGGAAACAGCATAGCAAGTTTAAATAAGGCTAGT | 396 | G78A | 0.98814561 |
| | CCGTTATCAACTTGAAAAAGTGGCACCGAATCGGTGCT | | | 2 |
| cr973 | GTTTAAGAGCTAAGCTGGAAACAGCATAGCAAGTTTAAATAAGGCTAGT | 397 | G62A | 0.98731444 |
| | CCGTTATCAACTTAAAAAAGTGGCACCGAGTCGGTGCT | | | 8 |
| cr671 | GTTTAAGAGCTAAGCTGGAAACAGCATAGCAAGTTTAAATAAGGCTAGT | 398 | A67G | 0.98643011 |
| | CCGTTATCAACTTGAAAAGGTGGCACCGAGTCGGTGCT | | | 9 |
| cr258 | GTTTAAGAGCTAAGCTGGAAACAGCATAGCAAGTTTAAATAAGGCTAGA | 399 | U48A | 0.98627815 |
| | CCGTTATCAACTTGAAAAAGTGGCACCGAGTCGGTGCT | | | |
| cr340 | GTTTAAGAGCTAAGCTGGAAACAGCATAGCAAGTTTAAATAAGGCTAGT | 400 | U61G | 0.98525699 |
| | CCGTTATCAACTGGAAAAAGTGGCACCGAGTCGGTGCT | | | 8 |
| cr578 | GTTTAAGAGCTAAGCTGGAAACAGCATAGCGAGTTTAAATAAGGCTAGT | 401 | A30G, | 0.98520091 |
| | CCGTTATCAACTTGATAAAGTGGCACCGAGTCGGTGCT | | A64U | |
| cr855 | GTTTAAGAGCTAAGCTGGAAACAGCATAGCAAGTTTAAATAAGGCCAGT | 402 | U45C, | 0.98491712 |
| | CCGTTCTCAACTTGACAAAGTGGCACCGAGTCGGTGCT | | A54C, | 2 |
| | | | A64C | |
| cr346 | GTTTAAGAGCTAAGCTGGAAACAGCATAGCAAGTTTAAATAAGGCTAGT | 403 | A58C, | 0.98472556 |
| | CCGTTATCACATTGAAAAATGGTCACCGAGTCGGTGAT | | C59A, | |
| | | | G68U, | |
| | | | U69G, | |
| | | | G71U, | |
| | | | C85A | |
| cr368 | GTTTAAGAGCTAAGCTGGAAACAGCATAGCAAGTTTAAATAAGGCTAGT | 404 | A58C, | 0.98362079 |
| | CCGTTATCACATTGAAAAATGGGCACCGAGTCGGTGCT | | C59A, | |
| | | | G68U, | |
| | | | U69G | |
| cr251 | GTTTAAGAGCTAAGCTGGAAACAGCATAGCAAGTTTAAATAAGGCTAGT | 405 | U53G, | 0.98359884 |
| | CCGTGATCAACTTGTAAAAGTGGCACCGACTCGGTGCT | | A63U, | 4 |
| | | | G78C | |
| cr270 | GTTTAAGAGCTAAGCTGGAAACAGCATAGCAAGTTTAAATAAGGCTAGT | 406 | A58U, | 0.98350452 |
| | CCGTTATCATCCTGAAAAGGAGGCACTGAGTCAGTGCT | | U60C, | |
| | | | A67G, | |
| | | | U69A, | |
| | | | C75U, | |
| | | | G81A | |
| cr970 | GTTTAAGAGCTAAGCTGGAAACAGCATAGCAAGTTTAAATAAGGCTAGT | 407 | A63U, | 0.98255639 |
| | CCGTTATCAACTTGTTAAAGTGGCACCGAGTCGGTGCT | | A64U | 1 |
| cr843 | GTTTAAGAGCTAAGCTGGAATCAGCATAGCAAGTTTAAATAAGGCTAGT | 408 | A20U | 0.98234868 |
| | CCGTTATCAACTTGAAAAAGTGGCACCGAGTCGGTGCT | | | |
| cr918 | GTTTAAGAGCTAAGCTGGAAACAGCATAGCAAGTTTAAATAAGGCTAGT | 409 | A58U, | 0.98202823 |
| | CCGTTATCATCTTGAAAAAGAGGCACCGAGTCGGTGCT | | U69A | 9 |
| cr906 | GTTTAAGAGCTAAGCTGGAAACAGCATAGCAAGTTTAAATAAGGCTAGT | 410 | A64G, | 0.98172688 |
| | CCGTTATCAACTTGAGAAAGTGGCAGCGAGTCGCTGCT | | C74G, | 6 |
| | | | G82C | |
| cr782 | GTTTAAGAGCTAAGCTGGAAACAGCATAGCAAGTTTAAATAAGGCTAGT | 411 | G62U, | 0.98164944 |
| | CCGTTATCAACTTTAGAAAGTGGCACCGAGTCGGTGCT | | A64G | 4 |
| cr969 | GTTTAAGAGCTAAGCTGGAAACAGCATAGCAAGTTTAAATAAGGCTAGT | 412 | A58U, | 0.98146492 |
| | CCGTTATCATCTTGAGAAAGAGGCACCGAGTCGGTGCT | | A64G, | 7 |
| | | | U69A | |
| cr747 | GTTTAAGAGCTAAGCTGGAAACAGCATAGCAAGTTTAAATAAGGCTAGT | 413 | A58C, | 0.98120572 |
| | CCGTTATCACCTTGAGAAAGGGGCACCGAGTCGGTGCT | | A64G, | 7 |
| | | | U69G | |
| cr926 | GTTTAAGAGCTAAGCTGGAAACAGAATAGCAAGTTTAAATAAGGCTAGT | 414 | C24A | 0.98110905 |
| | CCGTTATCAACTTGAAAAAGTGGCACCGAGTCGGTGCT | | | 3 |
| cr238 | GTTTAAGAGCTAAGCTGGAAACAGCATAGCAAGTTTAAATAAGGCTAGT | 415 | 73U, | 0.98091745 |
| | CCGTTATCAACTTGAAAAAGTGGCTTCGAGTCGAAGCT | | C74U, | 9 |
| | | | G82A, | |
| | | | U83A | |
| cr754 | GTTTAAGAGCTAAGCTGGAAACAGCATAGCAAGTTTAAATAAGGCTAGT | 416 | G62U | 0.98036311 |
| | CCGTTATCAACTTTAAAAAGTGGCACCGAGTCGGTGCT | | | 9 |
| cr811 | GTTTAAGAGCTAAGCTGGAAACAGTATAGCAAGTTTAAATAAGGCTAGT | 417 | C24U | 0.98025368 |
| | CCGTTATCAACTTGAAAAAGTGGCACCGAGTCGGTGCT | | | |
| cr624 | GTTTAAGAGCTAAGCTGGAAACAGCATAGCAAGTTTAAATAAGGCTAGT | 418 | A54G | 0.97985016 |
| | CCGTTGTCAACTTGAAAAAGTGGCACCGAGTCGGTGCT | | | 3 |
| cr226 | GTTTAAGAGCTAAGCTGGAAACAGCATAGCAAGTTTAAATAAGGCTAGT | 419 | A73C, | 0.97946311 |
| | CCGTTATCAACTTGAAAAAGTGGCCACGAGTCGTGGCT | | C74A, | 5 |
| | | | G82U, | |
| | | | U83G | |
| cr021 | GTTTAAGCGCTAAGCTGGAAACAGCATAGCAAGTTTAAATAAGGCTAGT | 420 | A7C | 0.97855226 |
| | CCGTTATCAACTTGAAAAAGTGGCACCGAGTCGGTGCT | | | 1 |
| cr556 | GTTTAAGAGCTAAGCTGGAAACAGCATAGCAAGTTTAAATAAGGCTAGT | 421 | U61C, | 0.97847942 |
| | CCGTTATCAACTCGAAAGAGTGATACCGAGTCGGTATT | | A66G, | 6 |
| | | | G71A, | |
| | | | C72U, | |
| | | | G84A, | |
| | | | C85U | |
| cr783 | GTTTAAGAGCTAAGCTGGAAACAGCATAGCAAGTTTAAATAAGGCTAGT | 422 | A58U, | 0.97788302 |
| | CCGTTATCATCTTGAAAAAGAGGAACCGAGTCGGTTCT | | U69A, | 9 |
| | | | C72A, | |
| | | | G84U | |
| cr224 | GTTTAAGAGCTAAGCTGGAAACAGCATAGCAAGTTTAAATAAGGCTAGT | 423 | U60C, | 0.97769106 |
| | CCGTTATCAACCTGAAAAGGTGGTATCGAGTCGATACT | | A67G, | 6 |
| | | | C72U, | |
| | | | C74U, | |
| | | | G82A, | |
| | | | G84A | |
| cr147 | GTTTAAGAGCTAAGCTGGAAACAGCATAGCAAGTTTAAATAAGGCTAGT | 424 | G62A, | 0.97534761 |
| | CCGTTATCAACTTATAAAAGTGGCACCGACTCGGTGCT | | A63U, | 5 |
| | | | G78C | |
| cr359 | GTTTAAGAGCTAAGCTGGAAACAGCATCGCAAGTTTAAATAAGGCTAGT | 425 | A27C | 0.97460129 |
| | CCGTTATCAACTTGAAAAAGTGGCACCGAGTCGGTGCT | | | 3 |
| cr307 | GTTTAAGAGCTAAGCTGGAAACAGCATAGCAAGTTTAAATAAGGCTAGT | 426 | G76A, | 0.97457656 |
| | CCGTTATCAACTTGAAAAAGTGGCACCAAGTTGGTGCT | | C80U | |
| cr159 | GTTTAAGAGCTAAGCTGGAAACAGCATAGCTAGTTTAAATAAGGCTAGT | 427 | A30U | 0.97393069 |
| | CCGTTATCAACTTGAAAAAGTGGCACCGAGTCGGTGCT | | | |
| cr800 | GTTTAAGAGCTAAGCTGGAAACAGCATAGCAAGTTTAAATAAGGTTAAT | 428 | C44U, | 0.97283385 |
| | CCGTTATCATCTTGAAAAAGAGACACCGAGTCGGTGTT | | G47A, | 3 |
| | | | A58U, | |
| | | | U69A, | |
| | | | G71A, | |
| | | | C85U | |
| cr299 | GTTTAAGAGCTAAGCTGGAAACAGCATAGCAAGTTTAAATAAGGCTAGT | 429 | A63G, | 0.97175695 |
| | CCGTTATCAACTTGGGAAAGTGGCACCGAGTCGGTGCT | | A64G | 4 |
| cr644 | GTTTAAGAGCTAAGCTGGAAACAGCATTGCAAGTTTAAATAAGGCTAGT | 430 | A27U | 0.96944571 |
| | CCGTTATCAACTTGAAAAAGTGGCACCGAGTCGGTGCT | | | 2 |
| cr825 | GTTTAAGAGCTAAGCTGGAAACAGCATAGCAAGTTTAAATAAGGCTAGT | 431 | A73U, | 0.96907861 |
| | CCGTTATCAACTTGAAAAAGTGGCTGCGAGTCGCAGCT | | C74G, | 4 |
| | | | G82C, | |
| | | | U83A | |
| cr287 | GTTTAAGAGCTAAGCTGGAAACAGCATAGCAAGTTTAAATAAGGCTAGT | 432 | A64U, | 0.96860250 |
| | CCGTTATCAACTTGATAAAGTGGCACCGACTCGGTGCT | | G78C | 8 |
| cr161 | GTTTAAGAGCTAAGCTGGAAACAGCATAGCAAGTTTAAATAAGGCTAGT | 433 | A65C, | 0.96712531 |
| | CCGTTATCAACTTGAACAAGTGGCACCGACTCGGTGCT | | G78C | 8 |
| cr994 | GTTTAAGAGCTAAGCTGGAAACAGCATAGCAAGTTTAAATAAGGCTAGT | 434 | G78U | 0.96707633 |
| | CCGTTATCAACTTGAAAAAGTGGCACCGATTCGGTGCT | | | 3 |
| cr102 | GTTTAAGAGCTAAGCTGGAAACAGCATAGCAAGTTTAAATAAGGCTAGT | 435 | C59U, | 0.96508051 |
| | CCGTTATCAATTTGAAAAAATGGAACCGAGTCGGTTCT | | G68A, | 1 |
| | | | C72A, | |
| | | | G84U | |
| cr306 | GTTTAAGAGCTAAGCTGGAAACAGCATAGCAAGTTTAAATAAGGCTTGT | 436 | A46U, | 0.96474194 |
| | CCGTTATCAACTTGAAAAAGTGGCACCGTGTCGGTGCT | | A77U | 1 |
| cr707 | GTTTAAGAGCTAAGCTGGAAACAGCATAGCAAGTTTAAATAAGGCTAGT | 437 | A77U | 0.96317072 |
| | CCGTTATCAACTTGAAAAAGTGGCACCGTGTCGGTGCT | | | 9 |
| cr831 | GTTTAAGAGCTAAGCTGGAAACAGCATAGCAAGTTTAAATAAGGCTAGT | 438 | C59U, | 0.96199445 |
| | CCGTTATCAATTTGAAAAAATGGCACCGAGTCGGTGCT | | G68A | 7 |
| cr646 | GTTTAAGAGCTAAGCTGGAAACAGCATAGCAAGTTTAAATAAGGCTAGT | 439 | C59A, | 0.96196816 |
| | CCGTTATCAAATTGAAAAATTGGCTCCGAGTCGGAGCT | | G68U, | 5 |
| | | | A73U, | |
| | | | U83A | |
| cr131 | GTTTAAGAGCTAAGCTGGAAACAGCATAGCAAGTTTAAATAAGGCTAGT | 440 | A64G, | 0.95996389 |
| | CCGTTATCAACTTGAGAAAGTGGCACAGAGTCTGTGCT | | C75A, | 7 |
| | | | G81U | |
| cr938 | GTTTAAGAGCTAAGCTGGAAACAGCATAGCAAGTTTAAATAAGGCTGGT | 441 | A46G | 0.95960505 |
| | CCGTTATCAACTTGAAAAAGTGGCACCGAGTCGGTGCT | | | 9 |
| cr416 | GTTTAAGAGCTAAGCTGGAAACAGCATAGCAAGTTTAAATAAGGCTAGT | 442 | A73G, | 0.95737807 |
| | CCGTTATCAACTTGAAAAAGTGGCGCCAAGTTGGCGCT | | G76A, | 5 |
| | | | C80U, | |
| | | | U83C | |
| cr267 | GTTTAAGAGCTAAGCTGCAAACAGCATAGCAAGTTTAAATAAGGCTAGT | 443 | G17C | 0.95520526 |
| | CCGTTATCAACTTGAAAAAGTGGCACCGAGTCGGTGCT | | | 8 |
| cr372 | GTTTAAGAGCTAAGCTGGAAACAGCATAGCAAGTTTAAATAAGGCGAGT | 444 | U45G | 0.95453841 |
| | CCGTTATCAACTTGAAAAAGTGGCACCGAGTCGGTGCT | | | 1 |
| cr167 | GTTTAAGAGCTAAGCTGGAAACAGCATAGCAAGTTTAAATAAGGCTAGT | 445 | U60C | 0.95445747 |
| | CCGTTATCAACCTGAAAAAGTGGCACCGAGTCGGTGCT | | | 1 |
| cr205 | GTTTAAGAGCTAAGCTGGAAACAGCATAGCAAGTTTAAATAAGGCTAGT | 446 | C59U, | 0.95153606 |
| | CCGTTATCAATTTGAAAAAATGGGACCGAGTCGGTCCT | | G68A, | 9 |
| | | | C72G, | |
| | | | G84C | |
| cr835 | GTTTAAGAGCTAAGCTGGAAACAGCATAGCAAGTTTAAATAAGGCTAGT | 447 | U83C | 0.95151510 |
| | CCGTTATCAACTTGAAAAAGTGGCACCGAGTCGGCGCT | | | 7 |
| cr264 | GTTTAAGAGCTAAGCTGGAAACAGCATAGCAAGTTTAAATAAGGCTAGT | 448 | A67U | 0.95129294 |
| | CCGTTATCAACTTGAAAATGTGGCACCGAGTCGGTGCT | | | 2 |
| cr397 | GTTTAAGAGCTAAGCTGGAAACAGCATAGCAAGTTTAAATAAGGCTAGT | 449 | C59A, | 0.95077636 |
| | CCGTTATCAAATTGAAAAATTGGCACCGAGTCGGTGCT | | G68U | 7 |
| cr181 | GTTTAAGAGCTAAGCTGGAAACAGCATAGCAAGTTTAAATAAGGTTAAT | 450 | C44U, | 0.95060466 |
| | CCGTTATCAATTTGAAAAAATGGCGCCGAGTCGGCGCT | | G47A, | 2 |
| | | | C59U, | |
| | | | G68A, | |
| | | | A73G, | |
| | | | U83C | |
| cr284 | GTTTAAGAGCTAAGCTGGAAACAGCATAGCAAGTTTAAATAAGGCTAGT | 451 | A64G, | 0.94972690 |
| | CCGTTATCAACTTGAGAAAGTGGCACCAAGTTGGTGCT | | G76A, | 6 |
| | | | C80U | |
| cr983 | GTTTAAGAGCTAAGCTGGAAACAGCATAGCAAGTTTAAATAAGGCTAGT | 452 | C72U | 0.94909651 |
| | CCGTTATCAACTTGAAAAAGTGGTACCGAGTCGGTGCT | | | 5 |
| cr529 | GTTTAAGAGCTAAGCTGGAAACAGCATAGCAAGTTTAAATAAGGCTAGT | 453 | C75U, | 0.94877517 |
| | CCGTTATCAACTTGAAAAAGTGGCACTGAGTCAGTGCT | | G81A | 3 |
| cr231 | GTTTAAGAGCTAAGCTGGAAACAGCATAGCAAGTTTAAATAAGGCTAGA | 454 | U48A, | 0.94853963 |
| | CCGTTATCAACTTGAGAAAGTGGCACCGAGTCGGTGCT | | A64G | 1 |
| cr703 | GTTTAAGAGCTAAGCTGGAAACAGCATAGCAAGTTTAAATAAGGCTAGT | 455 | A64G, | 0.94723877 |
| | CCGTTATCAACTTGAGGAAGTGGCACCGAGTCGGTGCT | | A65G | 5 |
| cr908 | GTTTAAGAGCTAAGCTGGAAACAGCATAGCAAGTTTAAATAAGGCTAGT | 456 | A64G, | 0.94547205 |
| | CCGTTATCAACTTGAGAAAGTGGCATCGAGTCGATGCT | | C74U, | 4 |
| | | | G82A | |
| cr285 | GTTTAAGAGCTAAGCTGGAAAAAGCATAGCAAGTTTAAATAAGGCTAGT | 457 | C21A | 0.94493325 |
| | CCGTTATCAACTTGAAAAAGTGGCACCGAGTCGGTGCT | | | 9 |
| cr718 | GTTTAAGAGCTAAGCTGGAAACAGCATAGCAAGTTTAAATAAGGCTAGT | 458 | A58U, | 0.94468358 |
| | CCGTTATCATCTTGAAAAAGAGGCCCCGAGTCGGGGCT | | U69A, | 4 |
| | | | A73C, | |
| | | | U83G | |
| cr142 | GTTTAAGAGCTAAGCTGGAAACAGCATAGCAAGTTTAAATAAGGCTAGT | 459 | G76U, | 0.94057837 |
| | CCGTTATCAACTTGAAAAAGTGGCACCTAGTAGGTGCT | | C80A | 2 |
| cr553 | GTTTAAGAGCTAAGCTGGAAACAGCATAGCAAGTTTAAATAAGGCTGGT | 460 | A46G, | 0.93851135 |
| | CCGTTATCAACTTGATAAAGTGGCACCGAGTCGGTGCT | | A64U | |
| cr253 | GTTTAAGAGCTAAGCTGGAAACAGCATAGCAAGTTTAAATAAGGCTAGT | 461 | A58G, | 0.93835701 |
| | CCGTTATCAGCCTGAAAAGGCGGCACCTAGTAGGTGCT | | U60C, | 8 |
| | | | A67G, | |
| | | | U69C, | |
| | | | G76U, | |
| | | | C80A | |
| cr719 | GTTTAAGAGCTAAGCTGGAAACAGCATAGCAAGTTTAAATAAGGCTAGT | 462 | C85A | 0.93811116 |
| | CCGTTATCAACTTGAAAAAGTGGCACCGAGTCGGTGAT | | | 1 |
| cr421 | GTTTAAGAGCTAAGCTGGAAACAGCATAGCAAGTTTAAATAAGGCTAGT | 463 | A67C | 0.93807843 |
| | CCGTTATCAACTTGAAAACGTGGCACCGAGTCGGTGCT | | | 9 |
| cr693 | GTTTAAGAGCTAAGCTGGAAACAGCATAGCAAGTTTAAATAAGGCTAGT | 464 | A73U, | 0.93692498 |
| | CCGTTATCAACTTGAAAAAGTGGCTCCAAGTTGGAGCT | | G76A, | |
| | | | C80U, | |
| | | | U83A | |
| cr823 | GTTTAAGAGCTAAGCTGGAAACAGCATAGCAAGTTTAAATAAGGCTAGT | 465 | U61A, | 0.93598690 |
| | CCGTTATCAACTAGAAATAGTGGCACTGAGTCAGTGCT | | A66U, | 9 |
| | | | C75U, | |
| | | | G81A | |
| cr371 | GTTTAAGAGCTAAGCTGGAAACAGCATAGCAAGTTTAAATAAGGCTAGT | 466 | U53C, | 0.93546240 |
| | CCGTCAGCAACTTGAAAAAGTGGCACCGACTCGGTGCT | | U55G, | 1 |
| | | | G78C | |
| cr576 | GTTTAAGAGCTAAGCCGGAAACAGCATAGCAAGTTTAAATAAGGCTAGT | 467 | U15C | 0.93498232 |
| | CCGTTATCAACTTGAAAAAGTGGCACCGAGTCGGTGCT | | | 3 |
| cr953 | GTTTAAGAGCTAAGCTGGAAACAGCATAGCAAGTTTAAATAAGGCTAGT | 468 | A77C | 0.93372576 |
| | CCGTTATCAACTTGAAAAAGTGGCACCGCGTCGGTGCT | | | 2 |
| cr822 | GTTTAAGAGCTAAGCTGGAAACAGCATAGCAAGTTTAAATAAGGCTAGA | 469 | U48A, | 0.93334098 |
| | CCGTTATCAACTTGAACAAGTGGCACCGAGTCGGTGCT | | A65C | |
| cr546 | GTTTAAGAGCTAAGCTGGAAACAGCATAGCAAGTTTAAATAAGGCTAGT | 470 | C59A, | 0.93261513 |
| | CCGTTATCAAATTGAGAAATTGGCACCGAGTCGGTGCT | | A64G, | 6 |
| | | | G68U | |
| cr630 | GTTTAAGAGCTAAGCTGGAAACAGCATAGCAAGTTTAAATAAGGCTAGT | 471 | A54G, | 0.93253594 |
| | CCGTTGCCAACTTGAAAAAGTGGCACCGACTCGGTGCT | | U55C, | 8 |
| | | | G78C | |
| cr291 | GTTTAAGAGCTAAGCTGGAAACAGCATAGCAAGTTTAAATAAGGCTAGT | 472 | G71C, | 0.93209066 |
| | CCGTTATCAACTTGAAAAAGTGCCATCGAGTCGATGGT | | C74U, | 5 |
| | | | G82A, | |
| | | | C85G | |
| cr243 | GTTTAAGAGCTAAGCTGGAAACAGCATAGCAAGTTTAAATAAGGCTAGT | 473 | U61C, | 0.93192234 |
| | CCGTTATCAACTCGAAAGAGTGGCCCTGAGTCAGGGCT | | A66G, | |
| | | | A73C, | |
| | | | C75U, | |
| | | | G81A, | |
| | | | U83G | |
| cr361 | GTTTAAGAGCTAAGCTCGAAACAGCATAGCAAGTTTAAATAAGGCTAGT | 474 | G16C | 0.93124453 |
| | CCGTTATCAACTTGAAAAAGTGGCACCGAGTCGGTGCT | | | 5 |
| cr577 | GTTTAAGAGCTAAGCTGGAAACAGCATGGCAAGTTTAAATAGGGCTAGT | 475 | A27G, | 0.93088485 |
| | CCGTTCTCAACTTGAAAAAGTGGCACCGAGTCGGTGCT | | A41G, | 2 |
| | | | A54C | |
| cr375 | GTTTAAGAGCTAAGCTTGAAACAGCATAGCAAGTTTAAATAAGGCTAGT | 476 | G16U | 0.92935273 |
| | CCGTTATCAACTTGAAAAAGTGGCACCGAGTCGGTGCT | | | 4 |
| cr780 | GTTTAAGAGCTAAGCTGGAAACAGCATAGCAAGTTTAAATAAGGCTAGT | 477 | U61G, | 0.92865664 |
| | CCGTTATCAACTGGAAACAGTGGCACCTAGTAGGTGCT | | A66C, | 4 |
| | | | G76U, | |
| | | | C80A | |
| cr304 | GTTTAAGAGCTAAGCTGGAAACAGCATAGCAAGTTTAAATAAGGCTAGT | 478 | C59U, | 0.92845293 |
| | CCGTTATCAATTTGAGAAAATGGCACCGAGTCGGTGCT | | A64G, | 2 |
| | | | G68A | |
| cr614 | GTTTAAGAGCTAAGCTGAAAACAGCATAGCAAGTTTAAATAAGGCTAGT | 479 | G17A | 0.92704840 |
| | CCGTTATCAACTTGAAAAAGTGGCACCGAGTCGGTGCT | | | 3 |
| cr769 | GTTTAAGAGCTAAGCTGGAAACAGCATAGCAGGTTTAAATAAGGCTAGT | 480 | A31G, | 0.92679541 |
| | CCGGAATCAACTTGAAAAAGTGGCACCGAGTCGGTGCT | | U52G, | 8 |
| | | | U53A | |
| cr974 | GTTTAAGAGCTAAGCTGGAAACAGCATAGCAAGTTTAAATAAGGCTAGT | 481 | A64G, | 0.92354510 |
| | CCGTTATCAACTTGAGAAAGTGGCACCGTGTCGGTGCT | | A77U | 7 |
| cr525 | GTTTAAGAGCTAAGCTGGAAACAGCATAGCAAGTTTAAATAAGGCTAGT | 482 | A58C, | 0.92054471 |
| | CCGTTATCACCTTGAAAAAGGGGCACGGAGTCCGTGCT | | U69G, | 6 |
| | | | C75G, | |
| | | | G81C | |
| cr752 | GTTTAAGAGCTAAGCTGGAAACAGCATAGCAAGTTTAAATAAGGCTAGT | 483 | A64U, | 0.91959212 |
| | CCGTTATCAACTTGATAAAGTGGCACCGAGTCGGTGCG | | U86G | 3 |
| cr132 | GTTTAAGAGCTAAGCTGGAAACAGCATAGCAAGTTTAAATAAGGCTAGT | 484 | A64G, | 0.91958452 |
| | CCGTTATCAACTTGAGAAAGTGGCACTGAGTCAGTGCT | | C75U, | 9 |
| | | | G81A | |
| cr364 | GTTTAAGAGCTAAGCTGGAAACAGCATAGCAAGTTTAAATAAGGCTAGT | 485 | U60A | 0.91926419 |
| | CCGTTATCAACATGAAAAAGTGGCACCGAGTCGGTGCT | | | 4 |
| cr388 | GTTTAAGAGCTAAGCTGGAAACAGCATAGCAAGTTTAAATAAGGCTAGT | 486 | G71U, | 0.91882856 |
| | CCGTTATCAACTTGAAAAAGTGTAACCGAGTCGGTTAT | | C72A, | 6 |
| | | | G84U, | |
| | | | C85A | |
| cr838 | GTTTAAGAGCTAAGCTGGAAACAGCATAGCAAGTTTAAATAAGGCCAGT | 487 | U45C | 0.91873492 |
| | CCGTTATCAACTTGAAAAAGTGGCACCGAGTCGGTGCT | | | 1 |
| cr597 | GTTTAAGAGCTAAGCTGGAAACAGCATAGCAAGTTTAAATAAGGCTAGT | 488 | A58C, | 0.91867601 |
| | CCGTTATCACCTTGAAAAAGGGGGACCTAGTAGGTCCT | | U69G, | 3 |
| | | | C72G, | |
| | | | G76U, | |
| | | | C80A, | |
| | | | G84C | |
| cr640 | GTTTAAGAGCTAAGCTGGAAACAGCATAGCAAGTTTAAATAAGGCTAGT | 489 | A73C, | 0.91863035 |
| | CCGTTATCAACTTGAAAAAGTGGCCCAGAGTCTGGGCT | | C75A, | 3 |
| | | | G81U, | |
| | | | U83G | |
| cr136 | GTTTAAGAGCTAAGCTGGAAACAGCATAGCAAGTTTAAATAAGGTTAAT | 490 | C44U, | 0.91716394 |
| | CCGTTATCAACTTGAAAAAGTGGCGACGAGTCGTCGCT | | G47A, | 7 |
| | | | A73G, | |
| | | | C74A, | |
| | | | G82U, | |
| | | | U83C | |
| cr910 | GTTTAAGAGCTAAGCTGGAAACAGCATAGCAAGTTTAAATAAGGCTAGT | 491 | C72A, | 0.91707468 |
| | CCGTTATCAACTTGAAAAAGTGGAACGGAGTCCGTTCT | | C75G, | 7 |
| | | | G81C, | |
| | | | G84U | |
| cr409 | GTTTAAGAGCTAAGCTGGAAAGAGCATAGCAAGTTTAAATAAGGCTAGT | 492 | C21G | 0.91575503 |
| | CCGTTATCAACTTGAAAAAGTGGCACCGAGTCGGTGCT | | | |
| cr977 | GTTTAAGAGCTAAGCTGGAAACAGCATAGCAAGTTTAAATAAGGCTAGT | 493 | A58U, | 0.91270854 |
| | CCGTTATCATCTTGAAAAAGAGGCACCAAGTTGGTGCT | | U69A, | 2 |
| | | | G76A, | |
| | | | C80U | |
| cr387 | GTTTAAGAGCTAAGCTGGAAACAGCATAGCAAGTTTAAATAAGGCTCGT | 494 | A46C | 0.90938800 |
| | CCGTTATCAACTTGAAAAAGTGGCACCGAGTCGGTGCT | | | 5 |
| cr503 | GTTTAAGAGCTAAGCTGGAAACAGCATAGCAAGTTTAAATAAGGCTAGT | 495 | U83G | 0.90794052 |
| | CCGTTATCAACTTGAAAAAGTGGCACCGAGTCGGGGCT | | | |
| cr863 | GTTTAAGAGCTAAGCTGGAAACCGCATAGCAAGTTTAAATAAGGCTAGT | 496 | A22C | 0.90310217 |
| | CCGTTATCAACTTGAAAAAGTGGCACCGAGTCGGTGCT | | | 8 |
| cr256 | GTTTAAGAGCTAAGCTGGAAACAGCATAACAAGTTTAAATAAGGCTAGT | 497 | G28A | 0.90225105 |
| | CCGTTATCAACTTGAAAAAGTGGCACCGAGTCGGTGCT | | | 7 |
| cr777 | GTTTAAGAGCTAAGCTGGAAACAGCATAGCAAGTTTAAATAAGGCTAGT | 498 | U52G | 0.90220219 |
| | CCGGTATCAACTTGAAAAAGTGGCACCGAGTCGGTGCT | | | 2 |
| cr141 | GTTTAAGAGCTAAGCTGGAAACAGCATAGCAAGTTTAAATAAGGCCAGT | 499 | U45C, | 0.90181817 |
| | CCGTTATCAACTTGAGAAAGTGGCACCGAGTCGGTGCT | | A64G | 1 |
| cr626 | GTTTAAGAGCTAAGCTGGAAACAGCATAGCAAGTTTAAATAAGGCTAGT | 500 | A64C, | 0.90061608 |
| | CCGTTATCAACTTGACAAAGTGGCACCGCGTCGGTGCT | | A77C | 7 |
| cr367 | GTTTAAGAGCTAAGCTGGAAACAGCATAGCAAGTTTAAATAAGACTAGT | 501 | G43A, | 0.89977091 |
| | TCGTTATCAACTCGAAAGAGTGGTACCGAGTCGGTACT | | C49U, | |
| | | | U61C, | |
| | | | A66G, | |
| | | | C72U, | |
| | | | G84A | |
| cr702 | GTTTAAGAGCTAAGCTGGAAACAGCATAGCAAGTTTAAATAAGGCTAGT | 502 | G71C | 0.89771066 |
| | CCGTTATCAACTTGAAAAAGTGCCACCGAGTCGGTGCT | | | 6 |
| cr402 | GTTTAAGAGCTAAGCTGGAAACTGCATAGCAAGTTTAAATAAGGCTAGT | 503 | A22U | 0.89765234 |
| | CCGTTATCAACTTGAAAAAGTGGCACCGAGTCGGTGCT | | | 3 |
| cr694 | GTTTAAGAGCTAAGCTGGAAACAGCATAGCAAGTTTAAATAAGGCTAGT | 504 | U52G, | 0.89629996 |
| | CCGGTATCAACTTGTAAAAGTGGCACCGAGTCGGTGCT | | A63U | 8 |
| cr206 | GTTTAAGAGCTAAGCTGGAAACAGCATAGCAAGTTTAAATAAGGCTAGT | 505 | U60C, | 0.89457114 |
| | CCGTTATCAACCTGAAAAAGTGGCACCGACTCGGTGCT | | G78C | 9 |
| cr013 | GTTTGAGAGCTAAGCTGGAAACAGCATAGCAAGTTTAAATAAGGCTAGT | 506 | A4G | 0.89069202 |
| | CCGTTATCAACTTGAAAAAGTGGCACCGAGTCGGTGCT | | | 7 |
| cr705 | GTTTAAGAGCTAAGCTGGAAACAGCATAGCAAGTTTAAATAAGGCTAGT | 507 | C75G, | 0.89014285 |
| | CCGTTATCAACTTGAAAAAGTGGCACGGAGTCCGTGCT | | G81C | 1 |
| cr520 | GTTTAAGAGCTAAGCTGGAAACAGCATAGCAAGTTTAAATAAGGCTAGT | 508 | U52A, | 0.88738904 |
| | CCGATATCAACTTGCAAAAGTGGCACCGAGTCGGTGCT | | A63C | 9 |
| cr123 | GTTTAAGAGCTAAGCTGGAAACAGCATAGCAAGTTTAAATAAGGCTAGT | 509 | U60G | 0.88706910 |
| | CCGTTATCAACGTGAAAAAGTGGCACCGAGTCGGTGCT | | | 6 |
| cr909 | GTTTAAGAGCTAAGCTGGAAACAGCATATCAAGTTTAAATAAGGCTAGT | 510 | G28U, | 0.88641919 |
| | CCGTGATCAACTTGTAAAAGTGGCACCGAGTCGGTGCT | | U53G, | 7 |
| | | | A63U | |
| cr869 | GTTTAAGAGCTAAGCTGGAAACAGCATAGCAAGTTTAAATAAGGCTAGT | 511 | U69A | 0.88612497 |
| | CCGTTATCAACTTGAAAAAGAGGCACCGAGTCGGTGCT | | | 4 |
| cr806 | GTTTAAGAGCTAAGCTGGAAACAGCATAGCAAGTTTAAATAGGGCTAGT | 512 | A41G | 0.88611665 |
| | CCGTTATCAACTTGAAAAAGTGGCACCGAGTCGGTGCT | | | 7 |
| cr358 | GTTTAAGAGCTAAGCTGGAAACAGCATAGCAAGTTTAAATAAGGTTAAT | 513 | C44U, | 0.88603636 |
| | CCGTTATCAAGTTGAAAAACTGGCACTGAGTCAGTGCT | | G47A, | |
| | | | C59G, | |
| | | | G68C, | |
| | | | C75U, | |
| | | | G81A | |
| cr984 | GTTTAAGAGCTAAGCTGGAAACAGCATAGCAAGTTTAAATAAGGCTAGT | 514 | C85G | 0.8857801 |
| | CCGTTATCAACTTGAAAAAGTGGCACCGAGTCGGTGGT | | | |
| cr749 | GTTTAAGAGCTAAGCTGGAAACAGCATAGCGAGTTTAAATAAGGCTAGT | 515 | A30G, | 0.88566025 |
| | CCGTCATCAACTTGAAAAAGTGGCACCGCGTCGGTGCT | | U53C, | 6 |
| | | | A77C | |
| cr414 | GTTTAAGAGCTAAGCTGGAAACAGCATAGCAAGTTTAAATAAGGCTAGT | 516 | A73U | 0.88521836 |
| | CCGTTATCAACTTGAAAAAGTGGCTCCGAGTCGGTGCT | | | 1 |
| cr286 | GTTTAAGAGCTAAGCTGGAAACAGCATAGCAAGTTTAAATAAGGCGAGT | 517 | U45G, | 0.88502424 |
| | CCGTTATCAACTTGAGAAAGTGGCACCGAGTCGGTGCT | | A64G | 9 |
| cr759 | GTTTAAGAGCTAAGCTGGAAACAGCATAGCAAGTTTAAATAAGGCTCGT | 518 | A46C, | 0.88424137 |
| | CCGTTATCAACTTGAGAAAGTGGCACCGAGTCGGTGCT | | A64G | 2 |
| cr396 | GTTTAAGAGCTAAGCTGGAAACAGCATAGCAAGTTTAAATAAGGCTAGT | 519 | C74A, | 0.88256931 |
| | CCGTTATCAACTTGAAAAAGTGGCAACAAGTTGTTGCT | | G76A, | 6 |
| | | | C80U, | |
| | | | G82U | |
| cr781 | GTTTAAGAGCTAAGCTGGAAACAGCATAGCAAGTTTAAATAAGGCTAGT | 520 | G84C | 0.88134119 |
| | CCGTTATCAACTTGAAAAAGTGGCACCGAGTCGGTCCT | | | 6 |
| cr729 | GTTTAAGAGCTAAGCTGGAAATAGCATAGCAAGTTTAAATAAGGCTAGT | 521 | C21U | 0.87934632 |
| | CCGTTATCAACTTGAAAAAGTGGCACCGAGTCGGTGCT | | | 5 |
| cr768 | GTTTAAGAGCTAAGCTGGAAACAGCATAGCAAGTTTAAATAAGGCTAGT | 522 | U79C | 0.87879335 |
| | CCGTTATCAACTTGAAAAAGTGGCACCGAGCCGGTGCT | | | 4 |
| cr236 | GTTTAAGAGCTAAGCTGGAAACAACATAGCAAGTTTAAATAAGGCTAGT | 523 | G23A | 0.87448856 |
| | CCGTTATCAACTTGAAAAAGTGGCACCGAGTCGGTGCT | | | 2 |
| cr260 | GTTTAAGAGCTAAGCTGGAAACAGCATAGCAAGTTTAAATAAGGCTAGT | 524 | U52A | 0.87399738 |
| | CCGATATCAACTTGAAAAAGTGGCACCGAGTCGGTGCT | | | 6 |
| cr153 | GTTTAAGAGCTAAGCTGGAAACAGCATAGCAAGTTTAAATAAGGCTAGT | 525 | C59A, | 0.87349549 |
| | CCGTTATCAAAATGAAAATTTGGCACCGAGTCGGTGCT | | U60A, | 7 |
| | | | A67U, | |
| | | | G68U | |
| cr991 | GTTTAAGAGCTAAGCTGGAAACAGCATAGCAAGTTTAAATAAGGCTAGT | 526 | U52A, | 0.8694336 |
| | CCGATATCAACTTGACAAAGTGGCACCGAGTCGGTGCT | | A64C | |
| cr105 | GTTTAAGAGCTAAGCTGGAAACAGCATAGCAAGTTTAAATAAGGCCAGT | 527 | U45C, | 0.86716970 |
| | CCGTTATCAACTTGACAAAGTGGCACCGAGTCGGTGCT | | A64C | 5 |
| cr692 | GTTTAAGAGCTAAGCTAGAAACAGCATAGCAAGTTTAAATAAGGCTAGT | 528 | G16A | 0.86645330 |
| | CCGTTATCAACTTGAAAAAGTGGCACCGAGTCGGTGCT | | | 7 |
| cr184 | GTTTAAGAGCTAAGCTGGAAACAGCATAGCAAGTTTAAATAAGGCTAGT | 529 | U52G, | 0.86534903 |
| | CCGGTATCAACTTGGAAAAGTGGCACCGAGTCGGTGCT | | A63G | 2 |
| cr216 | GTTTAAGAGCTAAGCTGGAAACAGCATAGCAAGTTTAAATAAGACTAGT | 530 | G43A, | 0.86518536 |
| | TCGTTATCAACTTGAAAAAGTGGCACCGAGTCGGTGCT | | C49U | 2 |
| cr643 | GTTTAAGAGCTAAGCTGGAAACAGCATAGCAAGTTTAAATAAGACTAGT | 531 | G43A, | 0.86495771 |
| | TCGTTATCAGCTTGAAAAAGCGGCATCGAGTCGATGCT | | C49U, | 7 |
| | | | A58G, | |
| | | | U69C, | |
| | | | C74U, | |
| | | | G82A | |
| cr891 | GTTTAAGAGCTAAGCTGGAAACAGCATAGCAAGTTTAAATAAGACTAGT | 532 | G43A, | 0.86406316 |
| | TCGTTATCAACTTGAAAAAGTGGTACCGAGTCGGTACT | | C49U, | 1 |
| | | | C72U, | |
| | | | G84A | |
| cr521 | GTTTAAGAGCTAAGCTGGAAACAGCATAGCAAGTTTAAATAAGGCTGGT | 533 | A46G, | 0.86375827 |
| | CCGTTATCAACTTGAAAAAGTGGCACCGTGTCGGTGCT | | A77U | 6 |
| cr865 | GTTTAAGAGCTAAGCTGGAAACAGCATAGCAAGTTTAAATAAGGCTAGT | 534 | A58U | 0.86372953 |
| | CCGTTATCATCTTGAAAAAGTGGCACCGAGTCGGTGCT | | | 9 |
| cr788 | GTTTAAGAGCTAAGCTGGAAACAGCATATCAAGTTTAAATAAGGCTAGT | 535 | G28U | 0.86173539 |
| | CCGTTATCAACTTGAAAAAGTGGCACCGAGTCGGTGCT | | | 8 |
| cr899 | GTTTAAGAGCTAAGCTGGAAACAGCATAGCAAGTTTAAATAAGACTAGT | 536 | G43A, | 0.85831302 |
| | TCGTTATCAGCTTGAAAAAGCGGTACCGAGTCGGTACT | | C49U, | |
| | | | A58G, | |
| | | | U69C, | |
| | | | C72U, | |
| | | | G84A | |
| cr829 | GTTTAAGAGCTAAGCTGGAAACAGCATACCAAGTTTAAATAAGGCTAGT | 537 | G28C | 0.85711850 |
| | CCGTTATCAACTTGAAAAAGTGGCACCGAGTCGGTGCT | | | 9 |
| cr676 | GTTTAAGAGCTAAGCTGGAAACAGCATAGCAAGTTTAAATAAGGCTAGT | 538 | A63G, | 0.85063962 |
| | CCGTTATCAACTTGGAAAAGTTGCACCGAGTCGGTGCT | | G70U | 2 |
| cr914 | GTTTAAGAGCTAAGCTGGAAACAGCATAGCAAGTTTAAATAAGACTAGT | 539 | G43A, | 0.84935014 |
| | TCGTTATCAACTTGAAAAAGTGGGACCGAGTCGGTCCT | | C49U, | 5 |
| | | | C72G, | |
| | | | G84C | |
| cr688 | GTTTAAGAGCTAAGCTGGAAACAGCATAGCAAGTTTAAATAAGGCTAGT | 540 | A64U, | 0.84797361 |
| | CCGTTATCAACTTGATAAAGTGGCACCGAGCCGGTGCT | | U79C | |
| cr878 | GTTTAAGAGCTAAGCTGGAAACAGCATAGCAAGTTTAAATAAGGTTAAT | 541 | C44U, | 0.84695212 |
| | CCGTTATCAATTTGAAAAAATGGCATCGAGTCGATGCT | | G47A, | 6 |
| | | | C59U, | |
| | | | G68A, | |
| | | | C74U, | |
| | | | G82A | |
| cr943 | GTTTAAGAGCTAAGCTGGAAACAGCATAGCAAGTTTAAATAAGGCTAGT | 542 | G71A, | 0.84643782 |
| | CCGTTATCAACTTGAAAAAGTGACAACGAGTCGTTGTT | | C74A, | 9 |
| | | | G82U, | |
| | | | C85U | |
| cr495 | GTTTAAGAGCTAAGCTGGAAACAGCATAGCAAGTTTAAATAAGGCTAGT | 543 | U79A | 0.84615752 |
| | CCGTTATCAACTTGAAAAAGTGGCACCGAGACGGTGCT | | | 7 |
| cr169 | GTTTAAGAGCTAAGCTGGAAACAGCATAGCAAGTTTAAATAAGGCTAGT | 544 | A64G, | 0.84535985 |
| | CCGTTATCAACTTGAGAAAGTGGCACCGAGCCGGTGCT | | U79C | 4 |
| cr960 | GTTTAAGAGCTAAGCTGGAAACAGCATAGCAAGTTTAAATAAGGCTAGT | 545 | A58U, | 0.84464285 |
| | CCGTTATCATATTGAAAAATAGGCACCGAGTCGGTGCT | | C59A, | 3 |
| | | | G68U, | |
| | | | U69A | |
| cr319 | GTTTAAGAGCTAAGCTGGAAACAGCATAGCAAGTTTAAATAGGGCTAGT | 546 | A41G, | 0.84187252 |
| | CCGTTATCAACTTGAGAAAGTGGCACCGAGTCGGTGCT | | A64G | 1 |
| cr370 | GTTTAAGAGCTAAGCTGGAAACAGCATAGCAAGTTTAAATAAGGCTAGT | 547 | A64G, | 0.84170145 |
| | CCGTTATCAACTTGAGAAAGTGGCACGGAGTCCGTGCT | | C75G, | 2 |
| | | | G81C | |
| cr647 | GTTTAAGAGCTAAGCTGGAAACAGGATAGCAAGTTTAAATAAGGCTAGT | 548 | C24G | 0.84082895 |
| | CCGTTATCAACTTGAAAAAGTGGCACCGAGTCGGTGCT | | | 5 |
| cr785 | GTTTAAGAGCTAAGCTGGAAACAGCATAGCAAGTTTAAATAAGGCGAGT | 549 | U45G, | 0.83777538 |
| | CCGTTATCAACTTGAAAAAGTGGCACCGAGTCGGTGCG | | U86G | 6 |
| cr590 | GTTTAAGAGCTAAGCTGGAAACAGCATAGCAAGTTTAAATAAGGCTAGT | 550 | A64C, | 0.83609198 |
| | CCGTTATCAACTTGACAAAGTGGCACCGAGCCGGTGCT | | U79C | 4 |
| cr554 | GTTTAAGAGCTAAGCTGGAAACAGCATAGCAGGTTTAAATAAGGCTAGT | 551 | A31G, | 0.83318759 |
| | CCGTTACCAACTTGAAAAAGTAGCACCGAGTCGGTGCT | | U55C, | 5 |
| | | | G70A | |
| cr816 | GTTTAAGAGCTAAGCTGGAAACAGCATAGCAAGTTTAAATAAGGCTAGT | 552 | G70U | 0.83153575 |
| | CCGTTATCAACTTGAAAAAGTTGCACCGAGTCGGTGCT | | | 4 |
| cr871 | GTTTAAGAGCTAAGCTGGAAACAGCATAGCAAGTTTAAATAAGCCTAGT | 553 | G43C, | 0.83130740 |
| | GCGTTATCAACTTGAAAAAGTGGCACCGAGTCGGTGCT | | C49G | 4 |
| cr435 | GTTTAAGAGCTAAGCTGGAAACAGCATAGCCAGTTTAAATAAGGCTAGT | 554 | A30C, | 0.82809025 |
| | CCGGTATCAACTTGAAAAAGTGGCACCGAGTCGGTGCT | | U52G | 7 |
| cr407 | GTTTAAGAGCTAAGCTGGAAACAGCATAGCAAGTTTAAATAAGCCTAGT | 555 | G43C, | 0.82775832 |
| | GCGTTATCAACCTGAAAAGGTGGCATCGAGTCGATGCT | | C49G, | 2 |
| | | | U60C, | |
| | | | A67G, | |
| | | | C74U, | |
| | | | G82A | |
| cr162 | GTTTAAGAGCTAAGCTGGAAACAGCATAGCAAGTTTAAATAAGGCTAGT | 556 | U83A | 0.82627090 |
| | CCGTTATCAACTTGAAAAAGTGGCACCGAGTCGGAGCT | | | 9 |
| cr543 | GTTTAAGAGCTAAGCTGGAAACAGCATAGCAAGTCTAAATAAGGCTAGT | 557 | U34C | 0.82305110 |
| | CCGTTATCAACTTGAAAAAGTGGCACCGAGTCGGTGCT | | | 8 |
| cr420 | GTTTAAGAGCTAAGCTGGAAACAGCATAGCAAGTTTAAATAAGGCTAGT | 558 | G71U, | 0.82289830 |
| | CCGTTATCAACTTGAAAAAGTGTGACCTAGTAGGTCAT | | C72G, | 8 |
| | | | G76U, | |
| | | | C80A, | |
| | | | G84C, | |
| | | | C85A | |
| cr601 | GTTTAAGAGCTAAGCTGGAAACAGCATAGAAAGTTTAAATAAGGCTAGT | 559 | C29A | 0.82162492 |
| | CCGTTATCAACTTGAAAAAGTGGCACCGAGTCGGTGCT | | | 5 |
| cr391 | GTTTAAGAGCTAAGCTGGAAACAGCATAGCAAGTTTAAATAAGCCTAGT | 560 | G43C, | 0.82156418 |
| | GCGTTATCAACTTGAAAAAGTGGCAACGAGTCGTTGCT | | C49G, | 5 |
| | | | C74A, | |
| | | | G82U | |
| cr362 | GTTTAAGAGCTAAGCTGGAAACAGCATAGCAAGTTTAAATAAGGCTAGT | 561 | C75A | 0.82137057 |
| | CCGTTATCAACTTGAAAAAGTGGCACAGAGTCGGTGCT | | | 4 |
| cr916 | GTTTAAGAGCTAAGCTGGAAACAGCATAGCAAGTTTAAATAAGACTAGT | 562 | G43A, | 0.82065028 |
| | TCGTTATCAACTTGAGAAAGTGGCACCGAGTCGGTGCT | | C49U, | 9 |
| | | | A64G | |
| cr697 | GTTTAAGAGCTAAGCTGGAAACAGCATTGCAAGTTTAAATAAGGCGAGT | 563 | A27U, | 0.81828644 |
| | CCGTTATCAACTTGCAAAAGTGGCACCGAGTCGGTGCT | | U45G, | 7 |
| | | | A63C | |
| cr621 | GTTTAAGAGCTAAGCTGGAAACAGCATAGCAAGTTTAAATAAGACTAGT | 564 | G43A, | 0.81795299 |
| | TCGTTATCAACGTGAAAACGTGGCACCAAGTTGGTGCT | | C49U, | 4 |
| | | | U60G, | |
| | | | A67C, | |
| | | | G76A, | |
| | | | C80U | |
| cr517 | GTTTAAGAGCTAAGCTGGAAACAGCATAGCAAGTTTAAATAAGGCTAGT | 565 | G81U | 0.81725880 |
| | CCGTTATCAACTTGAAAAAGTGGCACCGAGTCTGTGCT | | | 2 |
| cr740 | GTTTAAGAGCTAAGCTGGAAACAGCATAGCAAGTTTAAATAAGGCTAGT | 566 | G71C, | 0.81525923 |
| | CCGTTATCAACTTGAAAAAGTGCCAGCGAGTCGCTGGT | | C74G, | 6 |
| | | | G82C, | |
| | | | C85G | |
| cr911 | GTTTAAGAGCTAAGCTGGAAACAGCATAGCAAGTTTAAATAAGGCACGT | 567 | U45A, | 0.81010645 |
| | CCGTTATCAACTTGAAAAAGTGGCACCGAATCGGTGCT | | A46C, | 3 |
| | | | G78A | |
| cr470 | GTTTAAGAGCTAAGCTGGAAACATCATAGCAAGTTTAAATAAGGCTAGT | 568 | G23U | 0.80895739 |
| | CCGTTATCAACTTGAAAAAGTGGCACCGAGTCGGTGCT | | | 3 |
| cr678 | GTTTAAGAGCTAAGCTGGAAACAGCATAGCAAGTTTAAATAAGGCTAGT | 569 | G84A | 0.80811121 |
| | CCGTTATCAACTTGAAAAAGTGGCACCGAGTCGGTACT | | | 2 |
| cr506 | GTTTAAGAGCTAAGCTGGAAACAGCATAGCAAGTTTAAATAAGCCTAGT | 570 | G43C, | 0.80782074 |
| | GCGTTATCAACTTGAAAAAGTGGGACCGAGTCGGTCCT | | C49G, | 7 |
| | | | C72G, | |
| | | | G84C | |
| cr733 | GTTTAAGAGCTAAGCTGGAAACAGCATAGCAAGTTTAAATAAGGCTAGT | 571 | C74A, | 0.80699500 |
| | CCGTTATCAACTTGAAAAAGTGGCAAAGAGTCTTTGCT | | C75A, | 8 |
| | | | G81U, | |
| | | | G82U | |
| cr104 | GTTTAAGAGCTAAGCTGGAAACAGCATAGCAAGTTTAAATAAGGCTAGT | 572 | C72A | 0.80628936 |
| | CCGTTATCAACTTGAAAAAGTGGAACCGAGTCGGTGCT | | | 8 |
| cr215 | GTTTAAGAGCTAAGCTGGAAACAGCATAGCAAGTTTAAATAAGGCTAGT | 573 | G70A | 0.80585861 |
| | CCGTTATCAACTTGAAAAAGTAGCACCGAGTCGGTGCT | | | 8 |
| cr931 | GTTTAAGAGCTAAGCTGGAAACAGCATAGCAAGTTTAAATAAGGCTAGT | 574 | A73C, | 0.80442078 |
| | CCGTTATCAACTTGAAAAAGTGGCCCTGAGTCAGGGCT | | C75U, | 1 |
| | | | G81A, | |
| | | | U83G | |
| cr876 | GTTTAAGAGCTAAGCTGGAAACAGCATAGCAAGTTTAAATAAGGCTAGT | 575 | G81A | 0.80374722 |
| | CCGTTATCAACTTGAAAAAGTGGCACCGAGTCAGTGCT | | | 2 |
| cr905 | GTTTAAGAGCTAAGCTGGAAACAGCATAGCAAGTTTAAATAAGGCTAGT | 576 | G71A, | 0.80191610 |
| | CCGTTATCAACTTGAAAAAGTGATACCAAGTTGGTATT | | C72U, | 6 |
| | | | G76A, | |
| | | | C80U, | |
| | | | G84A, | |
| | | | C85U | |
| cr516 | GTTTAAGAGCTAAGCTGGAAACAGCATAGCAAGTTTAAATAAGCCTAGT | 577 | G43C, | 0.80091127 |
| | GCGTTATCAGCTTGAAAAAGCGGCACCGAGTCGGTGCT | | C49G, | 9 |
| | | | A58G, | |
| | | | U69C | |
| cr879 | GTTTAAGAGCTAAGCTGGAAACAGCATAGCAAGTTTAAATAAGGCGAGT | 578 | U45G, | 0.79803408 |
| | CCGTCAGCAACTTGAAAAAGTGGCACCGAGTCGGTGCT | | U53C, | 2 |
| | | | U55G | |
| cr484 | GTTTAAGAGCTAAGCTGGAAACAGCATAGCAAGTTTAAATAAGGCTAGT | 579 | G71C, | 0.79778173 |
| | CCGTTATCAACTTGAAAAAGTGCCACCAAGTTGGTGGT | | G76A, | 5 |
| | | | C80U, | |
| | | | C85G | |
| cr818 | GTTTAAGAGCTAAGCTGGAAACAGCATAGGAAGTTTAAATAAGGCTAGT | 580 | C29G | 0.79724822 |
| | CCGTTATCAACTTGAAAAAGTGGCACCGAGTCGGTGCT | | | 5 |
| cr652 | GTTTAAGAGCTAAGCTGGAAACAGCATCGCAAGTTTAAATAAGGCTAGT | 581 | A27C, | 0.79590116 |
| | CCGGTATCAACTTGGAAAAGTGGCACCGAGTCGGTGCT | | U52G, | 6 |
| | | | A63G | |
| cr199 | GTTTAAGAGCTAAGCTGGAAACAGCATAGCAAGTTTAAATAAGGCTAGT | 582 | A64G, | 0.79490499 |
| | CCGTTATCAACTTGAGAAAGTGGCACCTAGTAGGTGCT | | G76U, | |
| | | | C80A | |
| cr805 | GTTTAAGAGCTAAGCTGGAAACAGCATAGCAAGTTTAAATAGGGCTAGT | 583 | A41G, | 0.79274168 |
| | CCGTTATCAACTTGATAAAGTGGCACCGAGTCGGTGCT | | A64U | 5 |
| cr119 | GTTTAAGAGCTAAGCTGGAAACAGCATAGCAAGTTTAAATAAGGCTAGT | 584 | G68U | 0.79015629 |
| | CCGTTATCAACTTGAAAAATTGGCACCGAGTCGGTGCT | | | 5 |
| cr886 | GTTTAAGAGCTAAGCTGGAAACAGCATAGCAAGTTTAAATAAGGCTAGT | 585 | C59A, | 0.77914743 |
| | CCGTTATCAAATAGAAATATTGGCAGCGAGTCGCTGCT | | U61A, | 9 |
| | | | A66U, | |
| | | | G68U, | |
| | | | C74G, | |
| | | | G82C | |
| cr219 | GTTTAAGAGCTAAGCTGGAAACAGCATAGCAAGTTTAAATAAGCCTAGT | 586 | G43C, | 0.77785352 |
| | GCGTTATCAACTTGAGAAAGTGGCACCGAGTCGGTGCT | | C49G, | 7 |
| | | | A64G | |
| cr164 | GTTTAAGAGCTAAGCTGGAAACAGCATAGCAAGTTTAAATAAGGCTAGT | 587 | A73C | 0.77755220 |
| | CCGTTATCAACTTGAAAAAGTGGCCCCGAGTCGGTGCT | | | 8 |
| cr443 | GTTTAAGAGCTAAGCGGGAAACAGCATAGCAAGTTTAAATAAGGCTAGT | 588 | U15G | 0.77743236 |
| | CCGTTATCAACTTGAAAAAGTGGCACCGAGTCGGTGCT | | | 6 |
| cr712 | GTTTAAGAGCTAAGCTGGAAACACCATAGCAAGTTTAAATAAGGCTAGT | 589 | G23C | 0.77442100 |
| | CCGTTATCAACTTGAAAAAGTGGCACCGAGTCGGTGCT | | | 2 |
| cr449 | GTTTAAGAGCTAAGCTGGAAACAGCATAGCAAGTTTAAATAAGCCTAGT | 590 | G43C, | 0.76980003 |
| | GCGTTATCAACTTGAAAAAGTGGCAGCGAGTCGCTGCT | | C49G, | 1 |
| | | | C74G, | |
| | | | G82C | |
| cr558 | GTTTAAGAGCTAAGCTGGAAACAGCATAGCAAGTTTAAATAAGGCTAGT | 591 | A73U, | 0.76788418 |
| | CCGTTATCAACTTGAAAAAGTGGCTCGGAGTCCGAGCT | | C75G, | 5 |
| | | | G81C, | |
| | | | U83A | |
| cr550 | GTTTAAGAGCTAAGCTGGAAACAGCATAGCAAGTTTAAATAAGGCTAGT | 592 | C75A, | 0.76787138 |
| | CCGTTATCAACTTGAAAAAGTGGCACAAAGTTTGTGCT | | G76A, | |
| | | | C80U, | |
| | | | G81U | |
| cr684 | GTTTAAGAGCTAAGCTGGAAACAGCATAGCAAGTTTAAATAAGGCTAGT | 593 | C75U, | 0.76717563 |
| | CCGTTATCAACTTGAAAAAGTGGCACTAAGTTAGTGCT | | G76A, | 2 |
| | | | C80U, | |
| | | | G81A | |
| cr819 | GTTTAAGAGCTAAGCTGGAAACAGCATCGCAAGTTTAAATAAGGCTGGT | 594 | A27C, | 0.76666697 |
| | CCGGTATCAACTTGAAAAAGTGGCACCGAGTCGGTGCT | | A46G, | 5 |
| | | | U52G | |
| cr508 | GTTTAAGAGCTAAGCTGGAAACAGCATAGCAAGTTTAAATAAGGCCAGT | 595 | U45C, | 0.76359189 |
| | CCGTGATCAACTTGAAAAAGTGGCACCGAGCCGGTGCT | | U53G, | 1 |
| | | | U79C | |
| cr859 | GTTTAAGAGCTAAGCTGGAAACAGCATACCAAGTTTAAATAAGGCTTGT | 596 | G28C, | 0.76253874 |
| | CCGTTATCAACTTGAGAAAGTGGCACCGAGTCGGTGCT | | A46U, | 9 |
| | | | A64G | |
| cr836 | GTTTAAGAGCTAAGCTGGAAACAGCATAGCAAGTTTAAATAAGGCTAGT | 597 | C59U | 0.75741139 |
| | CCGTTATCAATTTGAAAAAGTGGCACCGAGTCGGTGCT | | | 6 |
| cr852 | GTTTAAGAGCTAAGCTGGAAACAGCATAGCAAGTTTAAATAAGGCTAGT | 598 | C59G, | 0.75416601 |
| | CCGTTATCAAGAAAAAATACTGGCACCGAGTCGGTGCT | | U60A, | 1 |
| | | | U61A, | |
| | | | G62A, | |
| | | | A66U, | |
| | | | G68C | |
| cr890 | GTTTAAGAGCTAAGCTGGAAACAGCATAGCAAGTTTAAATAAGGCTAGT | 599 | A77C, | 0.75125702 |
| | CCGTTATCAACTTGAAAAAGTGGCACCGCGTCGGTGCC | | U86C | 2 |
| cr779 | GTTTAAGAGCTAAGCTGGAAACAGCATAGCAAGTTTAAATAAGGCTAGT | 600 | C59A, | 0.75124325 |
| | CCGTTATCAAATTGAAAAATTGGTAACGAGTCGTTACT | | G68U, | 8 |
| | | | C72U, | |
| | | | C74A, | |
| | | | G82U, | |
| | | | G84A | |
| cr695 | GTTTAAGAGCTAAGCTGGAAACAGCATAGCAAGTTTAAATAAGGCTAGT | 601 | U52A, | 0.75099608 |
| | CCGATGTCAACTTGTAAAAGTGGCACCGAGTCGGTGCT | | A54G, | 4 |
| | | | A63U | |
| cr439 | GTTTAAGAGCTAAGCAGGAAACAGCATAGCAAGTTTAAATAAGGCTAGT | 602 | U15A | 0.75055046 |
| | CCGTTATCAACTTGAAAAAGTGGCACCGAGTCGGTGCT | | | 8 |
| cr887 | GTTTAAGAGCTAAGCTGGAAACAGCATAGCCAGTTTAAATAAGGCTCGT | 603 | A30C, | 0.75035737 |
| | CCGTTATCAACTTCAAAAAGTGGCACCGAGTCGGTGCT | | A46C, | 6 |
| | | | G62C | |
| cr867 | GTTTAAGAGCTAAGCTGGAAACAGCATAGCAAGTTTAAATAAGGCTAGT | 604 | C49U | 0.75021527 |
| | TCGTTATCAACTTGAAAAAGTGGCACCGAGTCGGTGCT | | | 5 |
| cr505 | GTTTAAGAGCTAAGCTGGAAACAGCATTGCAAGTTTAAATAGGGCTAGT | 605 | A27U, | 0.74590947 |
| | CCGTTATCAACTTGAAAAAGTGGCACCGATTCGGTGCT | | A41G, | 4 |
| | | | G78U | |
| cr875 | GTTTAAGAGCTAAGCTGGAAACAGCATAGCAAGTTTAAATAAGGCTAGT | 606 | A58C, | 0.74296049 |
| | CCGTTATCACCTGGAAACAGGGGCACCCAGTGGGTGCT | | U61G, | 8 |
| | | | A66C, | |
| | | | U69G, | |
| | | | G76C, | |
| | | | C80G | |
| cr594 | GTTTAAGAGCTAAGCTGGAAACAGCATTGCAAGTTTAAATAGGGCTAGT | 607 | A27U, | 0.73951937 |
| | CCGCTATCAACTTGAAAAAGTGGCACCGAGTCGGTGCT | | A41G, | |
| | | | U52C | |
| cr278 | GTTTAAGAGCTAAGCTGGAAACAGCATAGCAAGTTTAAATAAGGCTAGC | 608 | U48C, | 0.73676832 |
| | CCGTTATCAACTTGAAAAAGTGGCACCGAATCGGTGCT | | G78A | 8 |
| cr341 | GTTTAAGAGCTAAGCTGGAAACAGCATAGCAAGTTTAAATAAGTCTAGT | 609 | G43U, | 0.72986761 |
| | ACGTTATCAACTTGAAAAAGTGCTACCGAGTCGGTAGT | | C49A, | 2 |
| | | | G71C, | |
| | | | C72U, | |
| | | | G84A, | |
| | | | C85G | |
| cr426 | GTTTAAGAGCTAAGCTGGAAACAGCATAGCAAATTTAAATAAGGCTAGT | 610 | G32A, | 0.72645204 |
| | CCGCTATCAACTTGAAAAAGTGGCACCGAGTCGGTGCT | | U52C | |
| cr763 | GTTTAAGAGCTAAGCTGGAAACAGCATAGCAAGTTTAAATAAGGCTAGC | 611 | U48C | 0.72636557 |
| | CCGTTATCAACTTGAAAAAGTGGCACCGAGTCGGTGCT | | | 1 |
| cr474 | GTTTAAGAGCTAAGCTGGAAACAGCATAGCAAGTTTAAATAAGGCTAGT | 612 | G76U | 0.72544546 |
| | CCGTTATCAACTTGAAAAAGTGGCACCTAGTCGGTGCT | | | 3 |
| cr457 | GTTTAAGAGCTAAGCTGGAAACAGCATAGCAAGTTTAAATAAGGCTAGT | 613 | G84U | 0.72540834 |
| | CCGTTATCAACTTGAAAAAGTGGCACCGAGTCGGTTCT | | | 2 |
| cr168 | GTTTAAGAGCTAAGCTGGAAACAGCATAGCAAGTTTAAATAAGACTAGT | 614 | G43A, | 0.72384389 |
| | TCGTTATCAAATTGAAAAATTGGCACCGAGTCGGTGCT | | C49U, | 3 |
| | | | C59A, | |
| | | | G68U | |
| cr555 | GTTTAAGAGCTAAGCTGGAAACAGCATAGCAAGTTTAAATAAGTCTAGT | 615 | G43U, | 0.71899532 |
| | ACGTTATCAACTTGAAAAAGTGGCACCGAGTCGGTGCT | | C49A | 6 |
| cr645 | GTTTAAGAGCTAAGCTGGAAACAGCATAGCAAGTTTAAATAAGCCTAGT | 616 | G43C, | 0.71792415 |
| | GCGTTATCAACGTGAAAACGTGGCACGGAGTCCGTGCT | | C49G, | 9 |
| | | | U60G, | |
| | | | A67C, | |
| | | | C75G, | |
| | | | G81C | |
| cr635 | GTTTAAGAGCTAAGCTGGAAACAGCATAGCAAGTTTAAATAAGGCTAGT | 617 | A77G | 0.71742960 |
| | CCGTTATCAACTTGAAAAAGTGGCACCGGGTCGGTGCT | | | 7 |
| cr742 | GTTTAAGAGCTAAGCTGGAAACAGCATAGCAAGTTTAAATAAGGATATT | 618 | C44A, | 0.71407471 |
| | CCGTTATCAACTCGAAAGAGTGGCACCGAGTCGGTGCT | | G47U, | 9 |
| | | | U61C, | |
| | | | A66G | |
| cr539 | GTTTAAGAGCTAAGCTGGAAACAGCATAGCAAGTTTAAATAAGTCTAGT | 619 | G43U, | 0.71347240 |
| | ACGTTATCAATTGGAAACAATGGCACCGAGTCGGTGCT | | C49A, | 4 |
| | | | C59U, | |
| | | | U61G, | |
| | | | A66C, | |
| | | | G68A | |
| cr725 | GTTTAAGAGCTAAGCTGGAAACAGCATAGCAAATTTAAATAAGGCTAGT | 620 | G32A | 0.70944712 |
| | CCGTTATCAACTTGAAAAAGTGGCACCGAGTCGGTGCT | | | 1 |
| cr522 | GTTTAAGAGCTAAGCTGGAAACAGCATTGCAAGTTTAAATAAGGCTAGT | 621 | A27U, | 0.70838082 |
| | CCGTTGTCAACTTGAAAAAGTGGCACCGAGCCGGTGCT | | A54G, | 4 |
| | | | U79C | |
| cr015 | GTTTACGAGCTAAGCTGGAAACAGCATAGCAAGTTTAAATAAGGCTAGT | 622 | A5C | 0.70222589 |
| | CCGTTATCAACTTGAAAAAGTGGCACCGAGTCGGTGCT | | | 5 |
| cr117 | GTTTAAGAGCTAAGCTGGAAACAGCATAGCAAGTTTAAATAAGGCTAGT | 623 | A63G, | 0.69658802 |
| | CCGTTATCAACTTGGAAAAGTGGCACCGGGTCGGTGCT | | A77G | 2 |
| cr575 | GTTTAAGAGCTAAGCTGGAAACAGCATAGCAAGTTTAAATAAGGCTAGT | 624 | C75G | 0.69545400 |
| | CCGTTATCAACTTGAAAAAGTGGCACGGAGTCGGTGCT | | | 7 |
| cr734 | GTTTAAGAGCTAAGCTGGAAACAGCATCGCAAGTTTAAATAAGGCTAGT | 625 | A27C, | 0.69058295 |
| | CCGTTCTCAACTTGAAAAAGTCGCACCGAGTCGGTGCT | | A54C, | 8 |
| | | | G70C | |
| cr292 | GTTTAAGAGCTAAGCTGGAAACAGCATAGCAAGTTTAAATAAGGCTAGT | 626 | G70C | 0.69023779 |
| | CCGTTATCAACTTGAAAAAGTCGCACCGAGTCGGTGCT | | | 2 |
| cr596 | GTTTAAGAGCTAAGCTGGAAACAGCATAGCAAGTTTAAATAAGGCTAGT | 627 | C72G | 0.69002218 |
| | CCGTTATCAACTTGAAAAAGTGGGACCGAGTCGGTGCT | | | 6 |
| cr658 | GTTTAAGAGCTAAGCTGGAAACAGCATAGCAAGTTTAAATAAGGATATT | 628 | C44A, | 0.68825905 |
| | CCGTTATCAATTCGAAAGAATGGCACCGAGTCGGTGCT | | G47U, | 1 |
| | | | C59U, | |
| | | | U61C, | |
| | | | A66G, | |
| | | | G68A | |
| cr552 | GTTTAAGAGCTAAGCTGGAAACAGCATAGCAAGTTTAAATAAGGCTAGT | 629 | U52A, | 0.68635127 |
| | CCGATGGCAACTTGAAAAAGTGGCACCGAGTCGGTGCT | | A54G, | |
| | | | U55G | |
| cr877 | GTTTAAGAGCTAAGCTGGAAACAGCATAGCAAGTTTAAATAAGGCTAGT | 630 | U60C, | 0.68597396 |
| | CCGTTATCAACCTGAAAAGGTGCCACGGAGTCCGTGGT | | A67G, | 1 |
| | | | G71C, | |
| | | | C75G, | |
| | | | G81C, | |
| | | | C85G | |
| cr437 | GTTTAAGAGCTAAGCTGGAAACAGCATAGCAAGTTTAAATAAGGCTAGT | 631 | U60G, | 0.68540274 |
| | CCGTTATCAACGTGAAAACGTGGCACACAGTGTGTGCT | | A67C, | 8 |
| | | | C75A, | |
| | | | G76C, | |
| | | | C80G, | |
| | | | G81U | |
| cr607 | GTTTAAGAGCTAAGCTGGAAACAGCATAGCAAGTTTAAATAAGGCTAGT | 632 | G78A, | 0.68471269 |
| | CCGTTATCAACTTGAAAAAGTGGCACCGAACCGGTGCT | | U79C | 3 |
| cr279 | GTTTAAGAGCTAAGCTGGAAACAGCATAGCAAGTTTAAATAAGGCTAGT | 633 | U79C, | 0.67831028 |
| | CCGTTATCAACTTGAAAAAGTGGCACCGAGCCGGTGCC | | U86C | 7 |
| cr962 | GTTTAAGAGCTAAGCTGGAAACAGCATAGCAAGTTTAAATAAGGCTAGT | 634 | C72G, | 0.67332357 |
| | CCGTTATCAACTTGAAAAAGTGGGACCCAGTGGGTCCT | | G76C, | 2 |
| | | | C80G, | |
| | | | G84C | |
| cr273 | GTTTAAGAGCTAAGCTGGAAACAGCATAGCAAGTTTAAATAAGTCTAGT | 635 | G43U, | 0.67247738 |
| | ACGTTATCAACTTGAGAAAGTGGCACCGAGTCGGTGCT | | C49A, | 5 |
| | | | A64G | |
| cr848 | GTTTAAGAGCTAAGCTGGAAACAGCATAGCAAGTTTAAATAAGGCTAGT | 636 | A64G, | 0.67143035 |
| | CCGTTATCAACTTGAGAAAGTGGCACCGGGTCGGTGCT | | A77G | 4 |
| cr840 | GTTTAAGAGCTAAGCTGGAAACAGCATAGCAAGTTTAAATAAGGCTAGT | 637 | A64G, | 0.66880636 |
| | CCGTTATCAACTTGAGAAAGTAGCACCGAGTCGGTGCT | | G70A | 6 |
| cr133 | GTTTAAGAGCTAAGCTGGAAACAGCATAGCAAGTTTAAATAAGGCAAGC | 638 | U45A, | 0.66636251 |
| | CCGTTATCAACTTGAAAAAGTGGCACCGAGTCGGTGCT | | U48C | 5 |
| cr699 | GTTTAAGAGCTAAGCTGGAAACAGCATAGCAAGTTTAAATAAGGGTACT | 639 | C44G, | 0.66279088 |
| | CCGTTATCAACGCGAAAGCGTGGCACCGAGTCGGTGCT | | G47C, | 3 |
| | | | U60G, | |
| | | | U61C, | |
| | | | A66G, | |
| | | | A67C | |
| cr815 | GTTTAAGAGCTAAGCTGGAAACAGCATAGCAAGTTTAAATAAGGCTAGT | 640 | G82A | 0.65834857 |
| | CCGTTATCAACTTGAAAAAGTGGCACCGAGTCGATGCT | | | 4 |
| cr667 | GTTTAAGAGCTAAGCTGGAAACAGCATAGCAAGCTTAAATAAGGCTAGT | 641 | U33C, | 0.65332502 |
| | CCGTTATCAACTCGAAAGAGTGGCACCGAGTCGGTGCT | | U61C, | 9 |
| | | | A66G | |
| cr917 | GTTTAAGAGCTAAGCTGGAAACAGCATAGCAAGTTTAAATAAGGTTAAT | 642 | C44U, | 0.65213917 |
| | CCGTTATCAACTTGAAAAAGTGGCATCTAGTAGATGCT | | G47A, | 7 |
| | | | C74U, | |
| | | | G76U, | |
| | | | C80A, | |
| | | | G82A | |
| cr885 | GTTTAAGAGCTAAGCTGGAAACAGCATAGCCAGTTTAAATAAGGCTAGT | 643 | A30C, | 0.64981212 |
| | CCGTTATCAACTTGAAAAAGTTGCACCGAGTCGGTGCT | | G70U | 1 |
| cr017 | GTTTAGGAGCTAAGCTGGAAACAGCATAGCAAGTTTAAATAAGGCTAGT | 644 | A5G | 0.63898791 |
| | CCGTTATCAACTTGAAAAAGTGGCACCGAGTCGGTGCT | | | 7 |
| cr844 | GTTTAAGAGCTAAGCTGGAAACAGCATAGCAAGTTTAAATAAGGCTAGT | 645 | A58G, | 0.63456307 |
| | CCGTTATCAGCTTGAAAAAGCGGCATTGAGTCAATGCT | | U69C, | 7 |
| | | | C74U, | |
| | | | C75U, | |
| | | | G81A, | |
| | | | G82A | |
| cr793 | GTTTAAGAGCTAAGCTGGAAACAGCATAGCAAGTTTAAATAAGGCTAGT | 646 | G82U | 0.63432551 |
| | CCGTTATCAACTTGAAAAAGTGGCACCGAGTCGTTGCT | | | 1 |
| cr913 | GTTTAAGAGCTAAGCTGGAAACAGCATAGCAAGTTTAAACAAGGCTAGT | 647 | U39C | 0.63301621 |
| | CCGTTATCAACTTGAAAAAGTGGCACCGAGTCGGTGCT | | | 9 |
| cr213 | GTTTAAGAGCTAAGCTGGAAACAGCATAACAAGTTTAAATAGGGCTAGT | 648 | G28A, | 0.63252338 |
| | CCGTTATCAACTTGAGAAAGTGGCACCGAGTCGGTGCT | | A41G, | 3 |
| | | | A64G | |
| cr861 | GTTTAAGAGCTAAGCTGGAAACAGCATAGCAAGTTTAAATAAGGCTAGT | 649 | U55G, | 0.63185692 |
| | CCGTTAGCAACTTGAAAAAGTAGCACCGAGTCGGTGCT | | G70A | 8 |
| cr026 | GTTTGATAGCTAAGCTGGAAACAGCATAGCAAGTTTAAATAAGGCTAGT | 650 | A4G, | 0.6314288 |
| | CCGTTATCAACTTGAAAAAGTGGCACCGAGTCGGTGCT | | G6U | |
| cr001 | ATTTAAGAGCTAAGCTGGAAACAGCATAGCAAGTTTAAATAAGGCTAGT | 651 | G0A | 0.63118333 |
| | CCGTTATCAACTTGAAAAAGTGGCACCGAGTCGGTGCT | | | 7 |
| cr679 | GTTTAAGAGCTAAGCTGGAAACAGCATATCAAGTTTAAATAAGGCTAGT | 652 | G28U, | 0.62919092 |
| | CCGGTATCAACTTGAAAAAGTGGCACCGAGTCGGTGCT | | U52G | 7 |
| cr746 | GTTTAAGAGCTAAGCTGGAAACAGCATAGCAAATTTAAATAAGGCTAGT | 653 | G32A, | 0.62904932 |
| | CCGTTATCAACTTGACAAAGTGGCACCGAGTCGGTGCT | | A64C | 8 |
| cr406 | GTTTAAGAGCTAAGCTGGAAACAGCATAGCAAGTTTAAATAAGGCTAGT | 654 | C56U | 0.62852747 |
| | CCGTTATTAACTTGAAAAAGTGGCACCGAGTCGGTGCT | | | |
| cr189 | GTTTAAGAGCTAAGCTGGAAACAGCATAGCAAGTTTAAATAAGGCTAGT | 655 | G76A | 0.62702816 |
| | CCGTTATCAACTTGAAAAAGTGGCACCAAGTCGGTGCT | | | 6 |
| cr441 | GTTTAAGAGCTAAGCTGGAAACAGCATAGCAAGTTTAAATAAGGCTAGT | 656 | C56A | 0.62198502 |
| | CCGTTATAAACTTGAAAAAGTGGCACCGAGTCGGTGCT | | | |
| cr442 | GTTTAAGAGCTAAGCTGGAAACAGCATAGCAAGCTTAAATAAGGCTAGT | 657 | U33C, | 0.61690182 |
| | CCGTTATCAACCTGAAAAGGTGGCACCGAGTCGGTGCT | | U60C, | 3 |
| | | | A67G | |
| cr650 | GTTTAAGAGCTAAGCTGGAAACAGCATAGCAAGTTTAAATAAGGCTAGT | 658 | C56G | 0.61576590 |
| | CCGTTATGAACTTGAAAAAGTGGCACCGAGTCGGTGCT | | | 1 |
| cr086 | GTTTACTAGCTAAGCTGGAAACAGCATAGCAAGTTTAAATAAGGCTAGT | 659 | A5C, | 0.61399866 |
| | CCGTTATCAACTTGAAAAAGTGGCACCGAGTCGGTGCT | | G6U | 4 |
| cr937 | GTTTAAGAGCTAAGCTGGAAACAGCATAGCAAGTTTAAATAAGGGTACT | 660 | C44G, | 0.61374680 |
| | CCGTTATCAACTCGAAAGAGTGGCACAGAGTCTGTGCT | | G47C, | 3 |
| | | | U61C, | |
| | | | A66G, | |
| | | | C75A, | |
| | | | G81U | |
| cr227 | GTTTAAGAGCTAAGCTGGAAACAGCATAGCAAGTTTAAATAAGGCTAGT | 661 | U52C, | 0.60622290 |
| | CCGCTAACCACTTGAAAAAGTGGCACCGAGTCGGTGCT | | U55A, | 6 |
| | | | A57C | |
| cr639 | GTTTAAGAGCTAAGCTGGAAACAGCATAGCAAGTTTAAATAAGGATATT | 662 | C44A, | 0.60569626 |
| | CCGTTATCAACTTGAAAAAGTGGCACCGAGTCGGTGCT | | G47U | 4 |
| cr451 | GTTTAAGAGCTAAGCTGGAAACAGCATAGCAAGTTTAAATAAGGATATT | 663 | C44A, | 0.60528018 |
| | CCGTTATCAACTTGAAAAAGTGGCTCCGAGTCGGAGCT | | G47U, | 6 |
| | | | A73U, | |
| | | | U83A | |
| cr212 | GTTTAAGAGCTAAGCTGGAAACAGCATAGCAAATTTAAATAAGGCTAGT | 664 | G32A, | 0.60348445 |
| | CCGTTATCAACTTGAAGAAGTGGCACCGAGTCGGTGCT | | A65G | 5 |
| cr631 | GTTTAAGAGCTAAGCTGGAAACAGCATAGCAAGTTTAAATAAGGATATT | 665 | C44A, | 0.60140486 |
| | CCGTTATCAACATGAAAATGTGGCAACGAGTCGTTGCT | | G47U, | 5 |
| | | | U60A, | |
| | | | A67U, | |
| | | | C74A, | |
| | | | G82U | |
| cr479 | GTTTAAGAGCTAAGCTGGAAACAGCATAGCAAGTTTAAATAAGGCTAGT | 666 | C59G, | 0.59938023 |
| | CCGTTATCAAGTTGAAAAACTGGGACCCAGTGGGTCCT | | G68C, | 4 |
| | | | C72G, | |
| | | | G76C, | |
| | | | C80G, | |
| | | | G84C | |
| cr915 | GTTTAAGAGCTAAGCTGGAAACAGCATAGCAAGTTTAAATAAGGCTAGT | 667 | G76C, | 0.59653699 |
| | CCGTTATCAACTTGAAAAAGTGGCACCCAGTGGGTGCT | | C80G | |
| cr458 | GTTTAAGAGCTAAGCTGGAAACAGCATAGCAAGTTTAAATAAGGCTAGT | 668 | A64G, | 0.59579192 |
| | CCGTTATCAACTTGAGAAAGTCGCACCGAGTCGGTGCT | | G70C | 4 |
| cr008 | GTCTAAGAGCTAAGCTGGAAACAGCATAGCAAGTTTAAATAAGGCTAGT | 669 | U2C | 0.59551136 |
| | CCGTTATCAACTTGAAAAAGTGGCACCGAGTCGGTGCT | | | 5 |
| cr481 | GTTTAAGAGCTAAGCTGGAAACAGCATAGCAAGTTTAAATAAGGGTACT | 670 | C44G, | 0.59364768 |
| | CCGTTATCAACTTGAAAAAGTGACACCGAGTCGGTGTT | | G47C, | 2 |
| | | | G71A, | |
| | | | C85U | |
| cr598 | GTTTAAGAGCTAAGCTGGAAACAGCATAGCAAGCTTAAATAAGGCTAGT | 671 | U33C, | 0.59344375 |
| | CCGTTATCAACTGGAAACAGTGGCACCGAGTCGGTGCT | | U61G, | 8 |
| | | | A66C | |
| cr927 | GTTTAAGAGCTAAGCTGGAAACAGCATAGCAAGTTTAAATAAGGCTAGT | 672 | A73G, | 0.59205452 |
| | CCGTTATCAACTTGAAAAAGTGGCGCCCAGTGGGCGCT | | G76C, | 9 |
| | | | C80G, | |
| | | | U83C | |
| cr300 | GTTTAAGAGCTAAGCTGGAAACAGCATAGCAAGTTTAAATAAGGATATT | 673 | C44A, | 0.59198894 |
| | CCGTTATCAACTTGAAAAAGTGATACCGAGTCGGTATT | | G47U, | 1 |
| | | | G71A, | |
| | | | C72U, | |
| | | | G84A, | |
| | | | C85U | |
| cr726 | GTTTAAGAGCTAAGCTGGAAACAGCATAGCATGTTTAAATAAGGCTAGT | 674 | A31U, | 0.59028186 |
| | CCATTCTCAACTTGAAAAAGTGGCACCGAGTCGGTGCT | | G51A, | 5 |
| | | | A54C | |
| cr440 | GTTTAAGAGCTAAGCTGGAAACAGCATAGCAAGTTTAAATAAGGCTAGT | 675 | G68C | 0.58633892 |
| | CCGTTATCAACTTGAAAAACTGGCACCGAGTCGGTGCT | | | 4 |
| cr338 | GTTTAAGAGCTAAGCTGGAAACAGCATAGCAAGTCTAAATAAGGCGAGT | 676 | U34C, | 0.58305614 |
| | CCGTTATCAACTTGAAAAAGTGGCACCGAGTCGGTGCT | | U45G | 2 |
| cr456 | GTTTAAGAGCTAAGCTGGAAACAGCATAGCAAATTTAAATAAGGCAAGT | 677 | G32A, | 0.58211812 |
| | CCGTTATCAACTTGAAAAAGTGGCACCGAGTCGGTGCT | | U45A | 3 |
| cr827 | GTTTAAGAGCTAAGCTGGAAACAGCATAGCAAGTTTAAATAAGGATATT | 678 | C44A, | 0.57919268 |
| | CCGTTATCAACTTGAAAAAGTGGCTACGAGTCGTAGCT | | G47U, | 7 |
| | | | A73U, | |
| | | | C74A, | |
| | | | G82U, | |
| | | | U83A | |
| cr803 | GTTTAAGAGCTAAGCTGGAAACAGCATAGCAAGTTTAAATAAGGATATT | 679 | C44A, | 0.57804521 |
| | CCGTTATCAACTTGAGAAAGTGGCACCGAGTCGGTGCT | | G47U, | 6 |
| | | | A64G | |
| cr491 | GTTTAAGAGCTAAGCTGGAAACAGCATAGCAAGTTTAAATAAGGATATT | 680 | C44A, | 0.57750644 |
| | CCGTTATCAAGTTGAAAAACTGGCACCGAGTCGGTGCT | | G47U, | |
| | | | C59G, | |
| | | | G68C | |
| cr330 | GTTTAAGAGCTAAGCTGGAAACAGCATAGCAAGTTTAAATAAGGATATT | 681 | C44A, | 0.57616244 |
| | CCGTTATCAACTTGAAAAAGTGCCACCGAGTCGGTGGT | | G47U, | 9 |
| | | | G71C, | |
| | | | C85G | |
| cr500 | GTTTAAGAGCTAAGCTGGAAACAGCATAGCAAGTTTAAATAAGGCTAGT | 682 | U79G | 0.57426318 |
| | CCGTTATCAACTTGAAAAAGTGGCACCGAGGCGGTGCT | | | 5 |
| cr134 | GTTTAAGAGCTAAGCTGGAAACAGCATAGCAAGTTTAAATAAGGCTAGT | 683 | C56U, | 0.56922496 |
| | CCGTTATTAACTTGAACAAGTGGCACCGAGTCGGTGCT | | A65C | 8 |
| cr223 | GTTTAAGAGCTAAGCTGGAAACAGCATAGCAAGTTTAAATAAGGGTACT | 684 | C44G, | 0.56669424 |
| | CCGTTATCAACTTGAAAAAGTGGCACCGAGTCGGTGCT | | G47C | 5 |
| cr651 | GTTTAAGAGCTAAGCTGGAAACAGCATAGCAAGTTTAAATAAGGCTAGT | 685 | A54U, | 0.56370364 |
| | CCGTTTGCAACTTGAAAAAGTAGCACCGAGTCGGTGCT | | U55G, | |
| | | | G70A | |
| cr989 | GTTTAAGAGCTAAGCTGGAAACAGCATAGCAAGTTTAAATAAGGCTAGT | 686 | G68A | 0.56234333 |
| | CCGTTATCAACTTGAAAAAATGGCACCGAGTCGGTGCT | | | 4 |
| cr976 | GTTTAAGAGCTAAGCTGGAAACAGCATAGCAAGTTTAAATAAGGGTACT | 687 | C44G, | 0.55937371 |
| | CCGTTATCAACTTGAGAAAGTGGCACCGAGTCGGTGCT | | G47C, | 3 |
| | | | A64G | |
| cr957 | GTTTAAGAGCTAAGCTGGAAACAGCATAGCAAGTTTAAATAAGGGTACT | 688 | C44G, | 0.55763237 |
| | CCGTTATCAACTTGAAAAAGTGCCACCGAGTCGGTGGT | | G47C, | 3 |
| | | | G71C, | |
| | | | C85G | |
| cr533 | GTTTAAGAGCTAAGCTGGAAACAGCATAGCAAGTTTAAATAAGGCTAGT | 689 | A58G, | 0.55578553 |
| | CCGTTATCAGCTTGAAAAAGCGGCGGCGAGTCGCCGCT | | U69C, | 7 |
| | | | A73G, | |
| | | | C74G, | |
| | | | G82C, | |
| | | | U83C | |
| cr842 | GTTTAAGAGCTAAGCTGGAAACAGCATAGCAAGCTTAAATAAGGCTAGT | 690 | U33C, | 0.55441830 |
| | CCGTTATCAGCTTGAAAAAGCGGCACCGAGTCGGTGCT | | A58G, | 1 |
| | | | U69C | |
| cr837 | GTTTAAGAGCTAAGCTGGAAACAGCATAGCAAGTTTAAATAAGGCTAGT | 691 | G76C | 0.55433378 |
| | CCGTTATCAACTTGAAAAAGTGGCACCCAGTCGGTGCT | | | 8 |
| cr846 | GTTTAAGAGCTAAGCTGGAAACAGCATGGCAAGTTTAAATAAGGCTAGT | 692 | A27G, | 0.55275978 |
| | CCGTTATCAACTTAAAAAAGTGGCACCGGGTCGGTGCT | | G62A, | 5 |
| | | | A77G | |
| cr254 | GTTTAAGAGCTAAGCTGGAAACAGCATAGCAAGTTTAAATAAGGCTAGT | 693 | C75A, | 0.55028900 |
| | CCGTTATCAACTTGAAAAAGTGGCACACAGTGTGTGCT | | G76C, | 3 |
| | | | C80G, | |
| | | | G81U | |
| cr342 | GTTTAAGAGCTAAGCTGGAAACAGCATAGCAAGTTTAAATAAGGCTAGT | 694 | C56U, | 0.54224265 |
| | CCGTTATTAACTTGATAAAGTGGCACCGAGTCGGTGCT | | A64U | 1 |
| cr940 | GTTTAAGAGCTAAGCTGGAAACAGCATAGCAAGCTTAAATAAGGCTAGT | 695 | U33C, | 0.53990422 |
| | CCGTTATCAACGTGAAAACGTGGCACCGAGTCGGTGCT | | U60G, | 9 |
| | | | A67C | |
| cr427 | GTTTAAGAGCTAAGCTGGAAACAGCATAGCAAGTTTAAATAAGGCTAGT | 696 | C56A, | 0.53833838 |
| | CCGTTATAAACTTGAAAAAGTGGCACCGAATCGGTGCT | | G78A | 5 |
| cr098 | GTTTACGTGCTAAGCTGGAAACAGCATAGCAAGTTTAAATAAGGCTAGT | 697 | A5C, | 0.53404029 |
| | CCGTTATCAACTTGAAAAAGTGGCACCGAGTCGGTGCT | | A7U | 9 |
| cr430 | GTTTAAGAGCTAAGCTGGAAACAGCATAGCAAGTTTAAATAAGACTAGT | 698 | G43A, | 0.53277657 |
| | TCGTTATCAAATTGAAAAATTGGCACAGAGTCTGTGCT | | C49U, | 4 |
| | | | C59A, | |
| | | | G68U, | |
| | | | C75A, | |
| | | | G81U | |
| cr959 | GTTTAAGAGCTAAGCTGGAAACAGCATAGCAAGTTTAAATAAGGCTAGT | 699 | C59U, | 0.52511598 |
| | CCGTTATCAATTCGAAATACTGGCACCGAGTCGGTGCT | | U61C, | 8 |
| | | | A66U, | |
| | | | G68C | |
| cr511 | GTTTAAGAGCTAAGCTGGAAACAGCATTGCCAGTTTAAATAAGGCTAGT | 700 | A27U, | 0.52473745 |
| | CCGTTATCAACTTGAAAAAGTGGCACCGCGTCGGTGCT | | A30C, | 3 |
| | | | A77C | |
| cr166 | GTTTAAGAGCTAAGCTGGAAACAGCATAGCAAGCTTAAATAAGGCTAGT | 701 | U33C, | 0.52449451 |
| | CCGTTATCAACTTGAAAAAGTGGTACCGAGTCGGTACT | | C72U, | 6 |
| | | | G84A | |
| cr072 | GTCTAAGTGCTAAGCTGGAAACAGCATAGCAAGTTTAAATAAGGCTAGT | 702 | U2C, | 0.52361717 |
| | CCGTTATCAACTTGAAAAAGTGGCACCGAGTCGGTGCT | | A7U | 9 |
| cr150 | GTTTAAGAGCTAAGCTGGAAACAGCATAGCAAGTTTAAATAAGGCTAGT | 703 | C74G | 0.52298794 |
| | CCGTTATCAACTTGAAAAAGTGGCAGCGAGTCGGTGCT | | | 3 |
| cr724 | GTTTAAGAGCTAAGCTGGAAACAGCATAGCAAGTTTAAATAAGGCTAGT | 704 | A57G | 0.51772190 |
| | CCGTTATCGACTTGAAAAAGTGGCACCGAGTCGGTGCT | | | 4 |
| cr265 | GTTTAAGAGCTAAGCTGGAAACAGCATAGCAAGTTTAAATAAGGTTAGT | 705 | C44U | 0.51571786 |
| | CCGTTATCAACTTGAAAAAGTGGCACCGAGTCGGTGCT | | | 8 |
| cr548 | GTTTAAGAGCTAAGCTGGAAACAGCATAGCAAGTTTAAATAAGGCTAGT | 706 | C74A | 0.51302693 |
| | CCGTTATCAACTTGAAAAAGTGGCAACGAGTCGGTGCT | | | 4 |
| cr599 | GTTTAAGAGCTAAGCTGGAAACAGCATAGCAAGTTTAAATAAGGCTAGC | 707 | U48C, | 0.51123773 |
| | CCGTTATCAACGTGAAAAAGTGGCACCGAGTCGGTGCT | | U60G | |
| cr005 | GCTTAAGAGCTAAGCTGGAAACAGCATAGCAAGTTTAAATAAGGCTAGT | 708 | U1C | 0.51080241 |
| | CCGTTATCAACTTGAAAAAGTGGCACCGAGTCGGTGCT | | | 8 |
| cr255 | GTTTAAGAGCTAAGCTGGAAACAGCATAGCAAGCTTAAATAAGGCTAGT | 709 | U33C, | 0.50965183 |
| | CCGTTATCAACTTGAAAAAGTGGGACCGAGTCGGTCCT | | C72G, | 9 |
| | | | G84C | |
| cr675 | GTTTAAGAGCTAAGCTGGAAACAGCATAGCAAGCTTAAATAAGGCTAGT | 710 | U33C, | 0.50843344 |
| | CCGTTATCACCTTGAAAAAGGGGCACCGAGTCGGTGCT | | A58C, | 8 |
| | | | U69G | |
| cr659 | GTTTAAGAGCTAAGCTGGAAACAGCATAGCAAGTTTACATAAGGCTAGT | 711 | A37C | 0.50403513 |
| | CCGTTATCAACTTGAAAAAGTGGCACCGAGTCGGTGCT | | | 7 |
| cr190 | GTTTAAGAGCTAAGCTGGAAACAGCATAGCAAGTTTAAATAAGGCTAGT | 712 | U53A, | 0.50362754 |
| | CCGTAATTAACTTGAAAAAGTGGCACCGAGACGGTGCT | | C56U, | |
| | | | U79A | |
| cr317 | GTTTAAGAGCTAAGCTGGAAACAGCATAGCAAGTTTAAATATGGCTAGT | 713 | A41U | 0.50271410 |
| | CCGTTATCAACTTGAAAAAGTGGCACCGAGTCGGTGCT | | | 2 |
| cr120 | GTTTAAGAGCTAAGCTGGAAACAGCATAGCAAGTTTAAATAAGGCTAGT | 714 | A64G, | 0.5005408 |
| | CCGTTATCAACTTGAGAAAGTGGCACCCAGTGGGTGCT | | G76C, | |
| | | | C80G | |
| cr530 | GTTTAAGAGCTAAGCTGGAAACAGCATAGCAAGTTTAAATAAGGCTAGC | 715 | U48C, | 0.49980262 |
| | CCGATATCAACTTGAACAAGTGGCACCGAGTCGGTGCT | | U52A, | 2 |
| | | | A65C | |
| cr824 | GTTTAAGAGCTAAGCTGGAAACAGCATAGCAAGTTTAAATAAGGCTAGT | 716 | A64C, | 0.49899163 |
| | CCGTTATCAACTTGACAAAGTGGCACCGAGGCGGTGCT | | U79G | 6 |
| cr611 | GTTTAAGAGCTAAGCTGGAAACAGCATAGCAAGTTTAAATAAGGCTAGT | 717 | U55G, | 0.49537338 |
| | CCGTTAGAAACTTGAAAAAGTGGCACCGAATCGGTGCT | | C56A, | |
| | | | G78A | |
| cr765 | GTTTAAGAGCTAAGCTGGAAACAGCATAGCAAGTTTAAATAAGGCTAGT | 718 | U61G, | 0.49313142 |
| | CCGTTATCAACTGGAAACAGTGGCACTCAGTGAGTGCT | | A66C, | |
| | | | C75U, | |
| | | | G76C, | |
| | | | C80G, | |
| | | | G81A | |
| cr175 | GTTTAAGAGCTAAGCTGGAAACAGCATAGCAAGTTTAAATAAGGCTAGT | 719 | A57C | 0.49138007 |
| | CCGTTATCCACTTGAAAAAGTGGCACCGAGTCGGTGCT | | | 2 |
| cr016 | GTTTATGAGCTAAGCTGGAAACAGCATAGCAAGTTTAAATAAGGCTAGT | 720 | A5U | 0.48760843 |
| | CCGTTATCAACTTGAAAAAGTGGCACCGAGTCGGTGCT | | | 4 |
| cr620 | GTTTAAGAGCTAAGCTGGAAACAGCATAGCAAGTTTAAATAAGGGTACT | 721 | C44G, | 0.48424212 |
| | CCGTTATCAACTTGAAAAAGTGGAACTGAGTCAGTTCT | | G47C, | 7 |
| | | | C72A, | |
| | | | C75U, | |
| | | | G81A, | |
| | | | G84U | |
| cr682 | GTTTAAGAGCTAAGCTGGAAACAGCATAGCAAGTTTAAATAAGCGTACT | 722 | G43C, | 0.47942043 |
| | GCGTTATCAACTTGAAAAAGTGGCACCGAGTCGGTGCT | | C44G, | 2 |
| | | | G47C, | |
| | | | C49G | |
| cr770 | GTTTAAGAGCTAAGCTGGAAACAGCATAGCAAGCTTAAATAAGGCTAGT | 723 | U33C, | 0.47712207 |
| | CCGTTATCAACTTGAAAAAGTGGAACCGAGTCGGTTCT | | C72A, | 7 |
| | | | G84U | |
| cr177 | GTTTAAGAGCTAAGCTGGAAACAGCATAGCAAGTTTAAAGAAGGCTAGT | 724 | U39G | 0.47708504 |
| | CCGTTATCAACTTGAAAAAGTGGCACCGAGTCGGTGCT | | | 8 |
| cr124 | GTTTAAGAGCTAAGCTGGAAACAGCATAGCATGTTTAAATATGGCTAGT | 725 | A31U, | 0.47570216 |
| | CCGTTATCAACTTGAAAAAGTGGCACCGAGTCGGTGCT | | A41U | 2 |
| cr369 | GTTTAAGAGCTAAGCTGGAAACAGCATAGCAAGTTTAAATAAGGCTAGT | 726 | C59A | 0.47541190 |
| | CCGTTATCAAATTGAAAAAGTGGCACCGAGTCGGTGCT | | | 7 |
| cr071 | GTTTACGGGCTAAGCTGGAAACAGCATAGCAAGTTTAAATAAGGCTAGT | 727 | A5C, | 0.47500821 |
| | CCGTTATCAACTTGAAAAAGTGGCACCGAGTCGGTGCT | | A7G | 8 |
| cr493 | GTTTAAGAGCTAAGCTGGAAACAGCATAGCAAGTTTAAATAAGGCTAGC | 728 | U48C, | 0.47356427 |
| | CCGTTACAAACTTGAAAAAGTGGCACCGAGTCGGTGCT | | U55C, | 9 |
| | | | C56A | |
| cr399 | GTTTAAGAGCTAAGCTGGAAACAGCATAGCAAGTTTAAATAAGGCTAGT | 729 | G81C | 0.47270010 |
| | CCGTTATCAACTTGAAAAAGTGGCACCGAGTCCGTGCT | | | 7 |
| cr721 | GTTTAAGAGCTAAGCTGGAAACAGCATAGCAAGTTTAAATAAGGCTAGT | 730 | U52C, | 0.46963681 |
| | CCGCTATTAACTTGAAAAAGTGGCACCGAATCGGTGCT | | C56U, | 2 |
| | | | G78A | |
| cr904 | GTTTAAGAGCTAAGCTGGAAACAGCATAGCAAGTTTAAATAAGTCTAGT | 731 | G43U, | 0.46772125 |
| | ACGTTATCAATTTGAAAAAATGGCAACGAGTCGTTGCT | | C49A, | 2 |
| | | | C59U, | |
| | | | G68A, | |
| | | | C74A, | |
| | | | G82U | |
| cr163 | GTTTAAGAGCTAAGCTGGAAACAGCATAGCAAGCTTAAATAAGGCTAGT | 732 | U33C | 0.46620624 |
| | CCGTTATCAACTTGAAAAAGTGGCACCGAGTCGGTGCT | | | 2 |
| cr758 | GTTTAAGAGCTAAGCTGGAAACAGCATAGCAAGCTTAAATAAGGCTAGT | 733 | U33C, | 0.46135032 |
| | CCGCTATCAACTTGAAAAAGTGGCACCGAGTCGGTGCT | | U52C | 9 |
| cr405 | GTTTAAGAGCTAAGCTGGAAACAGCATAGCAAGTTTAAATAAGGCTAGT | 734 | G71U, | 0.45819411 |
| | CCGTTATCAACTTGAAAAAGTGTCAGCAAGTTGCTGAT | | C74G, | 6 |
| | | | G76A, | |
| | | | C80U, | |
| | | | G82C, | |
| | | | C85A | |
| cr920 | GTTTAAGAGCTAAGCTGGAAACAGCATAGCAAGTTTAAATAAGGCTAGT | 735 | A54G, | 0.45669801 |
| | CCGTTGACTACTTGAAAAAGTGGCACCGAGTCGGTGCT | | U55A, | 2 |
| | | | A57U | |
| cr873 | GTTTAAGAGCTAAGCTGGAAACAGCATAGCAAGCTTAAATAAGGCTAGT | 736 | U33C, | 0.45459228 |
| | CCGTTATCAACTAGAAATAGTGGCACCGAGTCGGTGCT | | U61A, | 1 |
| | | | A66U | |
| cr955 | GTTTAAGAGCTAAGCTGGAAACAGCATAGCAAGTTTAAATAAGGCTAAT | 737 | G47A | 0.45412799 |
| | CCGTTATCAACTTGAAAAAGTGGCACCGAGTCGGTGCT | | | 4 |
| cr868 | GTTTAAGAGCTAAGCTGGAAACAGCATAGCAAGTTCAAATAAGGCTAGT | 738 | U35C | 0.45170637 |
| | CCGTTATCAACTTGAAAAAGTGGCACCGAGTCGGTGCT | | | 1 |
| cr668 | GTTTAAGAGCTAAGCTGGAAACAGCATAGCAAGTTTAAATAAGGCTAGT | 739 | C59G | 0.44447612 |
| | CCGTTATCAAGTTGAAAAAGTGGCACCGAGTCGGTGCT | | | 8 |
| cr866 | GTTTAAGAGCTAAGCTGGAAACAGCATAGCAAGTTTAAATAAGGCTAGT | 740 | G71U, | 0.44339453 |
| | CCGTTATCAACTTGAAAAAGTGTCCCCGAGTCGGTGCT | | A73C | 8 |
| cr485 | GTTTAAGAGCTAAGCTGGAAACAGCATAGCAAGTTTAAATAAGGCTTGT | 741 | A46U, | 0.44190287 |
| | CCGTTATCAACTTGAAAAAGTTGCACCGTGTCGGTGCT | | G70U, | 5 |
| | | | A77U | |
| cr425 | GTTTAAGAGCTAAGCTGGAAACAGCATAGCAAGTTTAAATAAGGCTAGT | 742 | A58U, | 0.44091617 |
| | CCGTTATCATCCTGAAAATGCGGCACCGAGTCGGTGCT | | U60C, | 9 |
| | | | A67U, | |
| | | | U69C | |
| cr486 | GTTTAAGAGCTAAGCTGGAAACAGCATAGCAAGTTTAAATAAGGCTAGT | 743 | G71A, | 0.44090443 |
| | CCGTTATCAACTTGAAAAAGTGAAACTGAGTCAGTTTT | | C72A, | 2 |
| | | | C75U, | |
| | | | G81A, | |
| | | | G84U, | |
| | | | C85U | |
| cr460 | GTTTAAGAGCTAAGCTGGAAACAGCATAGCAAGTTTAAATAAGGGTACT | 744 | C44G, | 0.43813879 |
| | CCGTTATCAACTTGAAAAAGTGGCTCAGAGTCTGAGCT | | G47C, | 8 |
| | | | A73U, | |
| | | | C75A, | |
| | | | G81U, | |
| | | | U83A | |
| cr566 | GTTTAAGAGCTAAGCTGGAAACAGCATAGCAAGCTTAAATAAGGCTAGT | 745 | U33C, | 0.42991833 |
| | CCGTTATCAACATGAAAATUGGCACCGAGTCGGTGCT | | U60A, | 2 |
| | | | A67U | |
| cr148 | GTTTAAGAGCTAAGCTGGAAACAGCATAGCAAGCTTAAATAAGGCTAGT | 746 | U33C, | 0.42755995 |
| | CCGTTATCAACTTGAAAAAGTGGCGCCGAGTCGGCGCT | | A73G, | 3 |
| | | | U83C | |
| cr318 | GTTTAAGAGCTAAGCTGGAAACAGCATAGCAAGTTTAAATATGGCTAGT | 747 | A41U, | 0.42691626 |
| | CCGTTATCAACTTGATAAAGTGGCACCGAGTCGGTGCT | | A64U | 5 |
| cr040 | ATTTACGAGCTAAGCTGGAAACAGCATAGCAAGTTTAAATAAGGCTAGT | 748 | G0A, | 0.42325934 |
| | CCGTTATCAACTTGAAAAAGTGGCACCGAGTCGGTGCT | | ASC | 1 |
| cr165 | GTTTAAGAGCTAAGCTGGAAACAGCATAGCAAGTTTAAATAAAGCTAGT | 749 | G42A, | 0.42241109 |
| | CTGTTATCAACTCGAAAGAGTGTCACCGAGTCGGTGAT | | C50U, | 8 |
| | | | U61C, | |
| | | | A66G, | |
| | | | G71U, | |
| | | | C85A | |
| cr736 | GTTTAAGAGCTAAGCTGGAAACAGCATAGCAAGATTAAATAAGGCTAGT | 750 | U33A, | 0.42076454 |
| | CCGTTATCAACTTGAAAAAGTGGCACCGAGTCGGTGCA | | U86A | 1 |
| cr385 | GTTTAAGAGCTAAGCTGGAAACAGCATAGCAAGTTTAAATAAGGCTAGT | 751 | A73C, | 0.41856506 |
| | CCGTTATCAACTTGAAAAAGTGGCCACCAGTGGTGGCT | | C74A, | 6 |
| | | | G76C, | |
| | | | C80G, | |
| | | | G82U, | |
| | | | U83G | |
| cr728 | GTTTAAGAGCTAAGCTGGAAACAGCATAGCAAGTTTAAATAAGGCTAGT | 752 | A57G, | 0.41551803 |
| | CCGTTATCGACTTGACAAAGTGGCACCGAGTCGGTGCT | | A64C | 2 |
| cr794 | GTTTAAGAGCTAAGCTGGAAACAGCATAGCAAGTTTAAATAAGGCTAGT | 753 | G71C, | 0.41103318 |
| | CCGTTATCAACTTGAAAAAGTGCCACGGAGTCCGTGGT | | C75G, | 5 |
| | | | G81C, | |
| | | | C85G | |
| cr775 | GTTTAAGAGCTAAGCTGGAAACAGCATAGCGATTTTAAATAAGGCTAGT | 754 | A30G, | 0.40516743 |
| | CCGTTATCAACTTGAAAAAGTGGCACCGAGTCGGTGCT | | G32U | 1 |
| cr126 | GTTTAAGAGCTAAGCTGGAAACAGCATAGCAAATTTAAATAAGGCTCGT | 755 | G32A, | 0.40344846 |
| | CCGTTATCAACTTTAAAAAGTGGCACCGAGTCGGTGCT | | A46C, | 9 |
| | | | G62U | |
| cr799 | GTTTAAGAGCTAAGCTGGAAACAGCATAGCAAGTTTAAATAAGGCTAGT | 756 | C50U | 0.40310439 |
| | CTGTTATCAACTTGAAAAAGTGGCACCGAGTCGGTGCT | | | 5 |
| cr804 | GTTTAAGAGCTAAGCTGGAAACAGCATAGCAAGCTTAAATAAGGCTAGT | 757 | U33C, | 0.40252915 |
| | CCGTTATCAACTTGAAAAAGTGACACCGAGTCGGTGTT | | G71A, | 6 |
| | | | C85U | |
| cr569 | GTTTAAGAGCTAAGCTGGAAACAGCATGGCAAGTTTAAATAAGGCTCGT | 758 | A27G, | 0.39919149 |
| | CCGTTATGAACTTGAAAAAGTGGCACCGAGTCGGTGCT | | A46C, | 4 |
| | | | C56G | |
| cr545 | GTTTAAGAGCTAAGCTGGAAACAGCATAACAAGTTTAAATAAGGCTAGT | 759 | G28A, | 0.39355317 |
| | CCGTCATAAACTTGAAAAAGTGGCACCGAGTCGGTGCT | | U53C, | 3 |
| | | | C56A | |
| cr432 | GTTTAAGAGCTAAGCTGGAAACAGCATAGCAAGCTTAAATAAGGCTAGT | 760 | U33C, | 0.39234392 |
| | CCGTTATCAAGTTGAAAAACTGGCACCGAGTCGGTGCT | | C59G, | 8 |
| | | | G68C | |
| cr296 | GTTTAAGAGCTAAGCTGGAAACAGCATAGCAAGTTTAAATAAGGCTAGT | 761 | C72U, | 0.39110065 |
| | CCGTTATCAACTTGAAAAAGTGGTACGCAGTGCGTACT | | C75G, | 2 |
| | | | G76C, | |
| | | | C80G, | |
| | | | G81C, | |
| | | | G84A | |
| cr473 | GTTTAAGAGCTAAGCTGGAAACAGCATAGCAAGTTTAAATAAGGCTAGT | 762 | U52A, | 0.38921036 |
| | CCGATATAAACTTGCAAAAGTGGCACCGAGTCGGTGCT | | C56A, | 1 |
| | | | A63C | |
| cr249 | GTTTAAGAGCTAAGCTGGAAACAGCATAGCAAGTTTAAATAAGGCTAGT | 763 | U53G, | 0.38863339 |
| | CCGTGATTAACTTGAAAAAGTGGCACCGAGACGGTGCT | | C56U, | 8 |
| | | | U79A | |
| cr459 | GTTTAAGAGCTAAGCTGGAAACAGCATAGCGAGTTTAAATAAGGCTAGT | 764 | A30G, | 0.38369736 |
| | CCGTTATCTACTTGAAAAAGTGGCACCGAGTCGGTGCT | | A57U | 4 |
| cr856 | GTTTAAGAGCTAAGCTGGAAACAGCATAGCAAGTTTAAATAAGGATATT | 765 | C44A, | 0.38256316 |
| | CCGTTATCAAATTGAAAAATTGGCTCCGAGTCGGAGCT | | G47U, | 5 |
| | | | C59A, | |
| | | | G68U, | |
| | | | A73U, | |
| | | | U83A | |
| cr755 | GTTTAAGAGCTAAGCTGGAAACAGCATAGCAAGCTTAAATAAGGCTAGT | 766 | U33C, | 0.38143930 |
| | CCGTTATCAACTTGAAAAAGTGGCTCCGAGTCGGAGCT | | A73U, | 7 |
| | | | U83A | |
| cr813 | GTTTAAGAGCTAAGCTGGAAACAGCATAGCAAGTTTAAATAAGGCTAGT | 767 | A57U | 0.38026547 |
| | CCGTTATCTACTTGAAAAAGTGGCACCGAGTCGGTGCT | | | 8 |
| cr527 | GTTTAAGAGCTAAGCTGGAAACAGCATAGCAAGTGTAAATAAGGCTAGT | 768 | U34G | 0.38018693 |
| | CCGTTATCAACTTGAAAAAGTGGCACCGAGTCGGTGCT | | | 2 |
| cr200 | GTTTAAGAGCTAAGCTGGAAACAGCATAGCAAATTTAAATAAGGCTAGT | 769 | G32A, | 0.37991573 |
| | CCGTTATCAACTTGAAAAAGTGGCACCGAGTCGGTGCC | | U86C | 4 |
| cr404 | GTTTAAGAGCTAAGCTGGAAACAGCATAGCAAGTTTAAATAAGGCTAGT | 770 | A57C, | 0.37837378 |
| | CCGTTATCCACTTGATAAAGTGGCACCGAGTCGGTGCT | | A64U | 9 |
| cr523 | GTTTAAGAGCTAAGCTGGAAACAGCATAGCAAGCTTAAATAAGGCTAGT | 771 | U33C, | 0.37458916 |
| | CCGTTATCAACTTGAAAAAGTGTCACCGAGTCGGTGAT | | G71U, | 2 |
| | | | C85A | |
| cr088 | ATTTAGGAGCTAAGCTGGAAACAGCATAGCAAGTTTAAATAAGGCTAGT | 772 | G0A, | 0.37450544 |
| | CCGTTATCAACTTGAAAAAGTGGCACCGAGTCGGTGCT | | A5G | |
| cr343 | GTTTAAGAGCTAAGCTGGAAACAGCATAGCAAGTTTAAATAAGGCTAGT | 773 | A58U, | 0.37325235 |
| | CCGTTATCATGAAGAAAATGAGGCACCGAGTCGGTGCT | | C59G, | 1 |
| | | | U60A, | |
| | | | U61A, | |
| | | | A67U, | |
| | | | U69A | |
| cr201 | GTTTAAGAGCTAAGCTGGAAACAGCATAGCAAGTTTAAATAAAGCTAGT | 774 | G42A, | 0.37310290 |
| | CTGTTATCAACTTGAAAAAGTGGCACCGAGTCGGTGCT | | C50U | 4 |
| cr895 | GTTTAAGAGCTAAGCTGGAAACAGCATAGCAATTTTAAATAAGGCTAGT | 775 | G32U, | 0.37087207 |
| | CCGTTATCAACTTGTAAAAGTGGCACCGAGTCGGTGCT | | A63U | 5 |
| cr966 | GTTTAAGAGCTAAGCTGGAAACAGCATAGCAAGTTTAAATAAGGCTAGT | 776 | C59G, | 0.36991893 |
| | CCGTTATCAAGTTGAAAAACTGGCATGGAGTCCATGCT | | G68C, | 9 |
| | | | C74U, | |
| | | | C75G, | |
| | | | G81C, | |
| | | | G82A | |
| cr003 | TTTTAAGAGCTAAGCTGGAAACAGCATAGCAAGTTTAAATAAGGCTAGT | 777 | G0U | 0.36775399 |
| | CCGTTATCAACTTGAAAAAGTGGCACCGAGTCGGTGCT | | | 9 |
| cr398 | GTTTAAGAGCTAAGCTGGAAACAGCATAGCAAGTTTGAATAAGGCTAGT | 778 | A36G | 0.36671184 |
| | CCGTTATCAACTTGAAAAAGTGGCACCGAGTCGGTGCT | | | 7 |
| cr127 | GTTTAAGAGCTAAGCTGGAAACAGCATAGCAAGTTTAAATATGGCTAGT | 779 | A41U, | 0.36585642 |
| | CCGTTATCAACTTGAGAAAGTGGCACCGACTCGGTGCT | | A64G, | 6 |
| | | | G78C | |
| cr315 | GTTTAAGAGCTAAGCTGGAAACAGCATAGCAAGTTTAAATAAAGCTAGT | 780 | G42A, | 0.36416477 |
| | CTGTTATCAACTGGAAACAGTGGCCCCGAGTCGGGGCT | | C50U, | 3 |
| | | | U61G, | |
| | | | A66C, | |
| | | | A73C, | |
| | | | U83G | |
| cr924 | GTTTAAGAGCTAAGCTGGAAACAGCATAGCAAGTTTAAATAAAGCTAGT | 781 | G42A, | 0.36379822 |
| | CTGTTATCAACTTGAAAAAGTGGCGCCGAGTCGGCGCT | | C50U, | 5 |
| | | | A73G, | |
| | | | U83C | |
| cr196 | GTTTAAGAGCTAAGCTGGAAACAGCATAGCAAGTTTAAATAAGGCTAGT | 782 | C74G, | 0.36258355 |
| | CCGTTATCAACTTGAAAAAGTGGCAGCCAGTGGCTGCT | | G76C, | 4 |
| | | | C80G, | |
| | | | G82C | |
| cr471 | GTTTAAGAGCTAAGCTGGAAACAGCATAGCAAGTTTAAATAAGGCTAGT | 783 | G71U, | 0.36247118 |
| | CCGTTATCAACTTGAAAAAGTGTCACACAGTGTGTGAT | | C75A, | 5 |
| | | | G76C, | |
| | | | C80G, | |
| | | | G81U, | |
| | | | C85A | |
| cr192 | GTTTAAGAGCTAAGCTGGAAACAGCATAGCAAGTTTAAATAAGGCTAGT | 784 | A73C, | 0.36087432 |
| | CCGTTATCAACTTGAAAAAGTGGCCCCCAGTGGGGGCT | | G76C, | 6 |
| | | | C80G, | |
| | | | U83G | |
| cr252 | GTTTAAGAGCTAAGCTGGAAACAGCATAGCAAGTTTAAATAAGGCTAGT | 785 | G51A | 0.35809579 |
| | CCATTATCAACTTGAAAAAGTGGCACCGAGTCGGTGCT | | | 6 |
| cr182 | GTTTAAGAGCTAAGCTGGAAACAGCATAGCAATTTTAAATAAGGCTAGT | 786 | G32U | 0.35589435 |
| | CCGTTATCAACTTGAAAAAGTGGCACCGAGTCGGTGCT | | | 8 |
| cr351 | GTTTAAGAGCTAAGCTGGAAACAGCATAGCAAGTTTAAATAAGGCTCGT | 787 | A46C, | 0.35572605 |
| | CCGTTATAAACTTAAAAAAGTGGCACCGAGTCGGTGCT | | C56A, | |
| | | | G62A | |
| cr689 | GTTTAAGAGCTAAGCTGGAAACAGCATAGCAAGCTTAAATAAGGCTAGT | 788 | U33C, | 0.35467518 |
| | CCGTTTTCAACTTGATAAAGTGGCACCGAGTCGGTGCT | | A54U, | 3 |
| | | | A64U | |
| cr634 | GTTTAAGAGCTAAGCTGGAAACAGCATAGCAGGTTTAAATAAGGCTAGT | 789 | A31G, | 0.35083966 |
| | CCGTTATCGACTTGAAAAAGTGGCACCGAGTCGGTGCT | | A57G | |
| cr074 | GTTTATGCGCTAAGCTGGAAACAGCATAGCAAGTTTAAATAAGGCTAGT | 790 | A5U, | 0.34104410 |
| | CCGTTATCAACTTGAAAAAGTGGCACCGAGTCGGTGCT | | A7C | 7 |
| cr014 | GTTTCAGAGCTAAGCTGGAAACAGCATAGCAAGTTTAAATAAGGCTAGT | 791 | A4C | 0.33952866 |
| | CCGTTATCAACTTGAAAAAGTGGCACCGAGTCGGTGCT | | | 5 |
| cr064 | GTTTTAGAGCTAAGCTGGAAACAGCATAGCAAGTTTAAATAAGGCTAGT | 792 | A4U | 0.33827790 |
| | CCGTTATCAACTTGAAAAAGTGGCACCGAGTCGGTGCT | | | 6 |
| cr051 | TTTTAACAGCTAAGCTGGAAACAGCATAGCAAGTTTAAATAAGGCTAGTC | 793 | G0U, | 0.33671106 |
| | CGTTATCAACTTGAAAAAGTGGCACCGAGTCGGTGCT | | G6C | 9 |
| cr889 | GTTTAAGAGCTAAGCTGGAAACAGCATAGCAAGTTTAAATAAAGCTAGT | 794 | G42A, | 0.33467887 |
| | CTGTTATCAACTTGAAAAAGTGTCACCGAGTCGGTGAT | | C50U, | 4 |
| | | | G71U, | |
| | | | C85A | |
| cr349 | GTTTAAGAGCTAAGCTGGAAACAGCATAGCAAGTTTAAATAAGGCTAGT | 795 | C80U | 0.33263721 |
| | CCGTTATCAACTTGAAAAAGTGGCACCGAGTTGGTGCT | | | 2 |
| cr573 | GTTTAAGAGCTAAGCTGGAAACAGCATAGCTAGTTTAAATAAGGCTAGT | 796 | A30U, | 0.3296978 |
| | CCGATATCAACTTGAAAAAGTCGCACCGAGTCGGTGCT | | U52A, | |
| | | | G70C | |
| cr006 | GGTTAAGAGCTAAGCTGGAAACAGCATAGCAAGTTTAAATAAGGCTAGT | 797 | U1G | 0.32749112 |
| | CCGTTATCAACTTGAAAAAGTGGCACCGAGTCGGTGCT | | | 7 |
| cr242 | GTTTAAGAGCTAAGCTGGAAACAGCATAGCAAGTTTAAATAAGGGTACT | 798 | C44G, | 0.32442403 |
| | CCGTTATCAAGTTGAAAAACTGGCACCTAGTAGGTGCT | | G68C, | 1 |
| | | | G76U, | |
| | | | C80A | |
| cr069 | GCTTAAAAGCTAAGCTGGAAACAGCATAGCAAGTTTAAATAAGGCTAGT | 799 | U1C, | 0.32437081 |
| | CCGTTATCAACTTGAAAAAGTGGCACCGAGTCGGTGCT | | G6A | 5 |
| cr173 | GTTTAAGAGCTAAGCTGGAAACAGCATAGCAAGTTTAAATAAAGCTAGT | 800 | G42A, | 0.32435517 |
| | CTGTTATCAACTTGAAAAAGTGGTAGCGAGTCGCTACT | | C50U, | 4 |
| | | | C72U, | |
| | | | C74G, | |
| | | | G82C, | |
| | | | G84A | |
| cr897 | GTTTAAGAGCTAAGCTGGAAACAGCATAGCAAGTTTATATAAGGCTAGT | 801 | A37U | 0.32331910 |
| | CCGTTATCAACTTGAAAAAGTGGCACCGAGTCGGTGCT | | | 2 |
| cr850 | GTTTAAGAGCTAAGCTGGAAACAGCATAGCAAGTTTAAATAAGGCTAGT | 802 | A77G, | 0.32254175 |
| | CCGTTATCAACTTGAAAAAGTGGCACCGGGGCGGTGCT | | U79G | 8 |
| cr108 | GTTTAAGAGCTAAGCTGGAAACAGCATAGCAAGTTTAAATAAAGCTAGT | 803 | G42A, | 0.32214077 |
| | CTGTTATCAACTTGAGAAAGTGGCACCGAGTCGGTGCT | | C50U, | 5 |
| | | | A64G | |
| cr709 | GTTTAAGAGCTAAGCTGGAAACAGCATAGCAAGTTTAAATAAGGCTAGT | 804 | C56U, | 0.32107991 |
| | CCGTTATTAACTTGAAAAAGTTGCACCGAGTCGGTGCT | | G70U | 3 |
| cr468 | GTTTAAGAGCTAAGCTGGAAACAGCATAGCAAGATTAAATAAGGCTAGT | 805 | U33A | 0.3200489 |
| | CCGTTATCAACTTGAAAAAGTGGCACCGAGTCGGTGCT | | | |
| cr002 | CTTTAAGAGCTAAGCTGGAAACAGCATAGCAAGTTTAAATAAGGCTAGT | 806 | G0C | 0.3194695 |
| | CCGTTATCAACTTGAAAAAGTGGCACCGAGTCGGTGCT | | | |
| cr466 | GTTTAAGAGCTAAGCTGGAAACAGCATAGCAAGTTTAAAAAAGGCTAGT | 807 | U39A | 0.31532235 |
| | CCGTTATCAACTTGAAAAAGTGGCACCGAGTCGGTGCT | | | 2 |
| cr146 | GTTTAAGAGCTAAGCTGGAAACAGCATAGCAAGTTTAAATAAGGCTAGT | 808 | C75G, | 0.31411676 |
| | CCGTTATCAACTTGAAAAAGTGGCACGCAGTGCGTGCT | | G76C, | 1 |
| | | | C80G, | |
| | | | G81C | |
| cr210 | GTTTAAGAGCTAAGCTGGAAACAGCATAGCAAGTTTAAATAAGGCTAGT | 809 | A57U, | 0.31302175 |
| | CCGTTATCTACTTGACAAAGTGGCACCGAGTCGGTGCT | | A64C | 9 |
| cr004 | GATTAAGAGCTAAGCTGGAAACAGCATAGCAAGTTTAAATAAGGCTAGT | 810 | U1A | 0.31030084 |
| | CCGTTATCAACTTGAAAAAGTGGCACCGAGTCGGTGCT | | | 9 |
| cr055 | GGTTAAGCGCTAAGCTGGAAACAGCATAGCAAGTTTAAATAAGGCTAGT | 811 | U1G, | 0.30453876 |
| | CCGTTATCAACTTGAAAAAGTGGCACCGAGTCGGTGCT | | A7C | 7 |
| cr322 | GTTTAAGAGCTAAGCTGGAAACAGCATAGCAAGTTTAAATAAGGCTAGC | 812 | U48C, | 0.30330076 |
| | CCGTTATGAACTTGAAAAAGTGGCACCGAGTCGGTGCT | | C56G | 3 |
| cr209 | GTTTAAGAGCTAAGCTGGAAACAGCATAGCAAGTTTAAATCAGGCTAGT | 813 | A40C | 0.29592804 |
| | CCGTTATCAACTTGAAAAAGTGGCACCGAGTCGGTGCT | | | 5 |
| cr586 | GTTTAAGAGCTAAGCTGGAAACAGCATAGCAAGCTTAAATAAGGCTAGT | 814 | U33C, | 0.29391167 |
| | CCGTTATCAACTTGAAAAAGTGGCCCCGAGTCGGGGCT | | A73C, | 4 |
| | | | U83G | |
| cr591 | GTTTAAGAGCTAAGCTGGAAACAGCATAGCAAGTTTAAATAAGGCTAGT | 815 | A54G, | 0.29329260 |
| | CCGTTGTCTACTTGAAAAAGTGGCACCGAGTCGGTGCT | | A57U | 4 |
| cr027 | GTCTAAAAGCTAAGCTGGAAACAGCATAGCAAGTTTAAATAAGGCTAGT | 816 | U2C, | 0.28781518 |
| | CCGTTATCAACTTGAAAAAGTGGCACCGAGTCGGTGCT | | G6A | 3 |
| cr125 | GTTTAAGAGCTAAGCTGGAAACAGCATAGCAATTTTAAATAAGGCTAGT | 817 | G32U, | 0.28523061 |
| | CCGTTATCAACTTGATAAAGTGGCACCGAGTCGGTGCT | | A64U | 3 |
| cr237 | GTTTAAGAGCTAAGCTGGAAACAGCATAGCAAGTTTAAATAAGGATATT | 818 | C44A, | 0.28434303 |
| | CCGTTATCAACGTGAAAACGTGGCACCCAGTGGGTGCT | | G47U, | 6 |
| | | | U60G, | |
| | | | A67C, | |
| | | | G76C, | |
| | | | C80G | |
| cr982 | GTTTAAGAGCTAAGCTGGAAACAGCATAGCAAGCTTAAATAAGGCTAGT | 819 | U33C, | 0.28424175 |
| | CCGTTATCAACTTGAAAAAGTGGCAACGAGTCGTTGCT | | C74A, | 5 |
| | | | G82U | |
| cr344 | GTTTAAGAGCTAAGCTGGAAACAGCATAGCAAGTTTAAATAAGGCTAGT | 820 | G51A, | 0.28364044 |
| | CCATTATCAACTTGAATAAGTGGCACCGAGTCGGTGCT | | A65U | |
| cr331 | GTTTAAGAGCTAAGCTGGAAACAGCATAGCAAGCTTAAATAAGGCTAGT | 821 | U33C, | 0.28280001 |
| | CCGTTATCAACTTGAAAAAGTGGCATCGAGTCGATGCT | | C74U, | 5 |
| | | | G82A | |
| cr154 | GTTTAAGAGCTAAGCTGGAAACAGCATCGCAAGTTTAAATAAGGCTAGT | 822 | A27C, | 0.28265111 |
| | CCATTATCAACTTGAAAAAGTGGCACCGAGTCGGTGCT | | G51A | 6 |
| cr417 | GTTTAAGAGCTAAGCTGGAAACAGCATAGCAATTTTAAATAAGGCTAGT | 823 | G32U, | 0.28212921 |
| | CCGTTATCAACTTGAAGAAGTGGCACCGAGTCGGTGCT | | A65G | 7 |
| cr907 | GTTTAAGAGCTAAGCTGGAAACAGCATAGCAAGTTTAAATAAGGCTAGT | 824 | G51A, | 0.28205923 |
| | CCATTATCAACTTGTAAAAGTGGCACCGATTCGGTGCT | | A63U, | 2 |
| | | | G78U | |
| cr217 | GTTTAAGAGCTAAGCTGGAAACAGCATAGCAATTTTAAATAAGGCTAGT | 825 | G32U, | 0.28018899 |
| | CCGTTATCAACTTGACAAAGTGGCACCGAGTCGGTGCT | | A64C | 7 |
| cr992 | GTTTAAGAGCTAAGCTGGAAACAGCATTGCAAGTTTAAATAAGGCTAGT | 826 | A27U, | 0.27850684 |
| | CCATTATCAACTTGAAAAAGTGGCACCGAGTCGGTGCT | | G51A | 8 |
| cr246 | GTTTAAGAGCTAAGCTGGAAACAGCATAGCAAGTTTAAAAAAGGCTGGT | 827 | U39A, | 0.27791250 |
| | CCGTTATCAACTTGAAAAAGTGGCACCGAGTCGGTGCT | | A46G | 4 |
| cr972 | GTTTAAGAGCTAAGCTGGAAACAGCATAGCAAGTTTAAATAAGTGTACT | 828 | G43U, | 0.27672337 |
| | ACGTTATCAACCTGAAAAGGTGGCACCGAGTCGGTGCT | | C44G, | |
| | | | G47C, | |
| | | | C49A, | |
| | | | U60C, | |
| | | | A67G | |
| cr076 | AATTAAGAGCTAAGCTGGAAACAGCATAGCAAGTTTAAATAAGGCTAGT | 829 | G0A, | 0.27411866 |
| | CCGTTATCAACTTGAAAAAGTGGCACCGAGTCGGTGCT | | U1A | 7 |
| cr277 | GTTTAAGAGCTAAGCTGGAAACAGCATAGCAAGTTTAAATAAAGCTAGT | 830 | G42A, | 0.27320316 |
| | CTGTTATCAACTAGAAATAGTGGCACAGAGTCTGTGCT | | C50U, | 2 |
| | | | U61A, | |
| | | | A66U, | |
| | | | C75A, | |
| | | | G81U | |
| cr654 | GTTTAAGAGCTAAGCTGGAAACAGCATAGCAAGATTAAATAAGGCTAGT | 831 | U33A, | 0.26915994 |
| | CCGTTATCAACTTGATAAAGTGGCACCGAGTCGGTGCT | | A64U | |
| cr374 | GTTTAAGAGCTAAGCTGGAAACAGCATAGCAAGTTTAAATAAGGATAGT | 832 | C44A | 0.26856496 |
| | CCGTTATCAACTTGAAAAAGTGGCACCGAGTCGGTGCT | | | 7 |
| cr073 | GTTTTAGCGCTAAGCTGGAAACAGCATAGCAAGTTTAAATAAGGCTAGT | 833 | A4U, | 0.26502082 |
| | CCGTTATCAACTTGAAAAAGTGGCACCGAGTCGGTGCT | | A7C | 2 |
| cr046 | GGTTAACAGCTAAGCTGGAAACAGCATAGCAAGTTTAAATAAGGCTAGT | 834 | U1G, | 0.25916315 |
| | CCGTTATCAACTTGAAAAAGTGGCACCGAGTCGGTGCT | | G6C | 5 |
| cr965 | GTTTAAGAGCTAAGCTGGAAACAGCATAGCAAGTTTAAATAAGGGTACT | 835 | C44G, | 0.25715684 |
| | CCGTTATCAACTTGAAAAAGTGGCCTCGAGTCGAGGCT | | G47C, | 8 |
| | | | A73C, | |
| | | | C74U, | |
| | | | G82A, | |
| | | | U83G | |
| cr696 | GTTTAAGAGCTAAGCTGGAAACAGCATAGCGAGTTTAAATAAGGCTCGT | 836 | A30G, | 0.25531685 |
| | CCGTTATCCACTTGAAAAAGTGGCACCGAGTCGGTGCT | | A46C, | 6 |
| | | | A57C | |
| cr110 | GTTTAAGAGCTAAGCTGGAAACAGCATAGCAAGTTTAAATAAGGATATT | 837 | C44A, | 0.25473053 |
| | CCGTTATCAACTTGAAAAAGTGGCATTGAGTCAATGCT | | G47U, | 4 |
| | | | C74U, | |
| | | | C75U, | |
| | | | G81A, | |
| | | | G82A | |
| cr480 | GTTTAAGAGCTAAGCTGGAAACAGCATAGCAAGTTTAAATAAGGCTAGT | 838 | C59A, | 0.25432339 |
| | CCGTTATCAAATTGAAAAATTGACACCTAGTAGGTGTT | | G68U, | 5 |
| | | | G71A, | |
| | | | G76U, | |
| | | | C80A, | |
| | | | C85U | |
| cr077 | GATTAAGTGCTAAGCTGGAAACAGCATAGCAAGTTTAAATAAGGCTAGT | 839 | U1A, | 0.25379713 |
| | CCGTTATCAACTTGAAAAAGTGGCACCGAGTCGGTGCT | | A7U | 2 |
| cr662 | GTTTAAGAGCTAAGCTGGAAACAGCATAGCAAGTATAAATAAGGCTAGT | 840 | U34A | 0.24911940 |
| | CCGTTATCAACTTGAAAAAGTGGCACCGAGTCGGTGCT | | | 8 |
| cr720 | GTTTAAGAGCTAAGCTGGAAACAGCATAGCAAGCTTAAATAAGGTTAAT | 841 | U33C, | 0.24809349 |
| | CCGTTATCAACTTGAAAAAGTGGCACCGAGTCGGTGCT | | C44U, | 1 |
| | | | G47A | |
| cr792 | GTTTAAGAGCTAAGCTGGAAACAGCATAGCAAGCTTAAATAAGGCTAGT | 842 | U33C, | 0.24199913 |
| | CCGTTATCAACTTGAAAAAGTGCCACCGAGTCGGTGGT | | G71C, | 3 |
| | | | C85G | |
| cr158 | GTTTAAGAGCTAAGCTGGAAACAGCATAGCAAGTTTAAATAAAGTTAAT | 843 | G42A, | 0.24028055 |
| | CTGTTATCAACTCGAAAGAGTGGCACCGAGTCGGTGCT | | C44U, | 2 |
| | | | G47A, | |
| | | | C50U, | |
| | | | U61C, | |
| | | | A66G | |
| cr025 | CTTTAAAAGCTAAGCTGGAAACAGCATAGCAAGTTTAAATAAGGCTAGT | 844 | G0C, | 0.23960450 |
| | CCGTTATCAACTTGAAAAAGTGGCACCGAGTCGGTGCT | | G6A | 3 |
| cr403 | GTTTAAGAGCTAAGCTGGAAACAGCATAGCAAGCTTAAATAAGGCTAGT | 845 | U33C, | 0.23811666 |
| | CCGTTATCAACTTGAAAAAGTGGCACAGAGTCTGTGCT | | C75A, | 3 |
| | | | G81U | |
| cr198 | GTTTAAGAGCTAAGCTGGAAACAGCATAGCAATTTTAAATAAGGCTAGT | 846 | G32U, | 0.23597958 |
| | CCGTTATCAACTTGAAAAAGTGGCACCGAATCGGTGCT | | G78A | 8 |
| cr262 | GTTTAAGAGCTAAGCTGGAAACAGCATAGCAAGCTTAAATAAGGCTAGT | 847 | U33C, | 0.23293143 |
| | CCGTTATCATCTTGAAAAAGAGGCACCGAGTCGGTGCT | | A58U, | 4 |
| | | | U69A | |
| cr308 | GTTTAAGAGCTAAGCTGGAAACAGCATAGCAAGTTTAAATAAAGCTAGT | 848 | G42A, | 0.22487561 |
| | CTGTTATCATCTTGAAAAAGAGGCTCCGAGTCGGAGCT | | C50U, | 5 |
| | | | A58U, | |
| | | | U69A, | |
| | | | A73U, | |
| | | | U83A | |
| cr220 | GTTTAAGAGCTAAGCTGGAAACAGCATAGCAAGTTTAAATCAGGCTAGT | 849 | A40C, | 0.22292893 |
| | CCATAATCAACTTGAAAAAGTGGCACCGAGTCGGTGCT | | G51A, | 8 |
| | | | U53A | |
| cr207 | GTTTAAGAGCTAAGCTGGAAACAGCATAGCAAGTTTAAATAAGGCTAGT | 850 | C80G | 0.21537753 |
| | CCGTTATCAACTTGAAAAAGTGGCACCGAGTGGGTGCT | | | 8 |
| cr276 | GTTTAAGAGCTAAGCTGGAAACAGCATAGCAAGTTTAAATAAGGATATT | 851 | C44A, | 0.21429568 |
| | CCGTTATCAGCTTGAAAAAGCGGCACCCAGTGGGTGCT | | G47U, | 9 |
| | | | A58G, | |
| | | | U69C, | |
| | | | G76C, | |
| | | | C80G | |
| cr853 | GTTTAAGAGCTAAGCTGGAAACAGCATAGCAATTTTAAATAAGGCGAGT | 852 | G32U, | 0.21213594 |
| | CCGTTATCAACTTGAAAAAGTGGCACCGAGTCGGTGCT | | U45G | 2 |
| cr194 | GTTTAAGAGCTAAGCTGGAAACAGCATAGCAAGTTTAAATAAGGCTAGT | 853 | C49A | 0.21128789 |
| | ACGTTATCAACTTGAAAAAGTGGCACCGAGTCGGTGCT | | | 4 |
| cr854 | GTTTAAGAGCTAAGCTGGAAACAGCATAGCAAGTTTAAATAAGGCTAGT | 854 | C74G, | 0.20988350 |
| | CCGTTATCAACTTGAAAAAGTGGCAGGGAGTCCCTGCT | | C75G, | 8 |
| | | | G81C, | |
| | | | G82C | |
| cr011 | GTTCAAGAGCTAAGCTGGAAACAGCATAGCAAGTTTAAATAAGGCTAGT | 855 | U3C | 0.20883537 |
| | CCGTTATCAACTTGAAAAAGTGGCACCGAGTCGGTGCT | | | 4 |
| cr180 | GTTTAAGAGCTAAGCTGGAAACAGCATAGCAAGTTTAAATCAGGCTAGT | 856 | A40C, | 0.20797546 |
| | CCGTTATCAACTTGAAAAAGTGGCACCGAGCCGGTGCT | | U79C | 7 |
| cr218 | GTTTAAGAGCTAAGCTGGAAACAGCATAGCAATTTTAAATAAGGCTAGT | 857 | G32U, | 0.20699910 |
| | CCGTTATCAACTTAAACAAGTGGCACCGAGTCGGTGCT | | G62A, | 3 |
| | | | A65C | |
| cr373 | GTTTAAGAGCTAAGCTGGAAACAGCATAGCAAGCTTAAATAAGGCTAGT | 858 | U33C, | 0.19930318 |
| | CCGTTATCAACTTGAAAAAGTGGCAGCGAGTCGCTGCT | | C74G, | 7 |
| | | | G82C | |
| cr429 | GTTTAAGAGCTAAGCTGGAAACAGCATAGCAAGTTTAAATAAGAATATT | 859 | G43A, | 0.19596394 |
| | TCGTTATCAACTTGAAAAAGTGGCAACGAGTCGTTGCT | | C44A, | 3 |
| | | | G47U, | |
| | | | C49U, | |
| | | | C74A, | |
| | | | G82U | |
| cr732 | GTTTAAGAGCTAAGCTGGAAACAGCATAGCAAGTTTAAATAGGGCTAGT | 860 | A41G, | 0.19257399 |
| | CCGGTATCAACTTGAAAAAGTCGCACCGAGTCGGTGCT | | U52G, | 8 |
| | | | G70C | |
| cr347 | GTTTAAGAGCTAAGCTGGAAACAGCATAGCAAGCTTAAATAAGCCTAGT | 861 | U33C, | 0.19150948 |
| | GCGTTATCAACTTGAAAAAGTGGCACCGAGTCGGTGCT | | G43C, | 9 |
| | | | C49G | |
| cr760 | GTTTAAGAGCTAAGCTGGAAACAGCATAGCAGGTTTAAATCAGGCTAGT | 862 | A31G, | 0.19086803 |
| | CCGTTATCAACTTGATAAAGTGGCACCGAGTCGGTGCT | | A40C, | 4 |
| | | | A64U | |
| cr294 | GTTTAAGAGCTAAGCTGGAAACAGCATAGCAAGTTTAAATAAGAATATT | 863 | G43A, | 0.18828983 |
| | TCGTTATCAACTTGAAAAAGTGGCACCGAGTCGGTGCT | | C44A, | 3 |
| | | | G47U, | |
| | | | C49U | |
| cr090 | GTTTTATAGCTAAGCTGGAAACAGCATAGCAAGTTTAAATAAGGCTAGT | 864 | A4U, | 0.18647663 |
| | CCGTTATCAACTTGAAAAAGTGGCACCGAGTCGGTGCT | | G6U | 3 |
| cr900 | GTTTAAGAGCTAAGCTGGAAACAGCATAGCAAGTTGAAATAAGGCTAGT | 865 | U35G | 0.18459068 |
| | CCGTTATCAACTTGAAAAAGTGGCACCGAGTCGGTGCT | | | 6 |
| cr605 | GTTTAAGAGCTAAGCTGGAAACAGCATAGCAAGTTTAAATAAGGATATT | 866 | C44A, | 0.18385681 |
| | CCGTTATCAACTTGAAAAAGTGGCACCCAGTGGGTGCT | | G47U, | 5 |
| | | | G76C, | |
| | | | C80G | |
| cr452 | GTTTAAGAGCTAAGCTGGAAACAGCATAGCAAATTTAAATAAGGCTAGT | 867 | G32A, | 0.18130680 |
| | CCGTTATAAACTTGAAAAAGTGGCACCGAGTCGGTGCT | | C56A | 9 |
| cr311 | GTTTAAGAGCTAAGCTGGAAACAGCATAGCAAGTTTAAATAAGGCTTGT | 868 | A46U, | 0.17902707 |
| | CCGTTATATACTTGAAAAAGTGGCACCGAGTCGGTGCT | | C56A, | 1 |
| | | | A57U | |
| cr812 | GTTTAAGAGCTAAGCTGGAAACAGCATAGCAAGTTTAAATAAGGCTACT | 869 | G47C | 0.17658719 |
| | CCGTTATCAACTTGAAAAAGTGGCACCGAGTCGGTGCT | | | |
| cr979 | GTTTAAGAGCTAAGCTGGAAACAGCATAGCAAGTTTAAATAAGGCTAGT | 870 | U52G, | 0.17650523 |
| | CCGGTATCTACTTGAAAAAGTGGCACCGAGTCGGTGCT | | A57U | 7 |
| cr185 | GTTTAAGAGCTAAGCTGGAAACAGCATAGCAAGTTTAAATAAGGCTAGT | 871 | C80A | 0.17551027 |
| | CCGTTATCAACTTGAAAAAGTGGCACCGAGTAGGTGCT | | | 6 |
| cr047 | GTTTTAGTGCTAAGCTGGAAACAGCATAGCAAGTTTAAATAAGGCTAGT | 872 | A4U, | 0.16858817 |
| | CCGTTATCAACTTGAAAAAGTGGCACCGAGTCGGTGCT | | A7U | 9 |
| cr029 | GTTTCAGGGCTAAGCTGGAAACAGCATAGCAAGTTTAAATAAGGCTAGT | 873 | A4C, | 0.16628472 |
| | CCGTTATCAACTTGAAAAAGTGGCACCGAGTCGGTGCT | | A7G | 8 |
| cr826 | GTTTAAGAGCTAAGCTGGAAACAGCATAGCAATTTTAAATAAGGCTAGC | 874 | G32U, | 0.16327899 |
| | CCGTTTTCAACTTGAAAAAGTGGCACCGAGTCGGTGCT | | U48C, | 8 |
| | | | A54U | |
| cr447 | GTTTAAGAGCTAAGCTGGAAACAGCATAGCAAGCTTAAATAAGGCTAGT | 875 | U33C, | 0.16159763 |
| | CCGTTATCAACTTGAAAAAGTGGCACCAAGTTGGTGCT | | G76A, | 4 |
| | | | C80U | |
| cr544 | GTTTAAGAGCTAAGCTGGAAACAGCATAGCAACTTTAAATAAGGCTAGT | 876 | G32C, | 0.16051593 |
| | CCGTTATCAACTCGAAAGAGTGGCACCGAGTCGGTGCT | | U61C, | 6 |
| | | | A66G | |
| cr230 | GTTTAAGAGCTAAGCTGGAAACAGCATAACAAGTTTAAATAAGGCTAGT | 877 | G28A, | 0.15978141 |
| | CCATTATCAACTTGAACAAGTGGCACCGAGTCGGTGCT | | G51A, | |
| | | | A65C | |
| cr618 | GTTTAAGAGCTAAGCTGGAAACAGCATAGCAAGTTTAAGTAAGGCTAGT | 878 | A38G | 0.15709594 |
| | CCGTTATCAACTTGAAAAAGTGGCACCGAGTCGGTGCT | | | 5 |
| cr683 | GTTTAAGAGCTAAGCTGGAAACAGCATAGCAAGTTTAAATAAGGCTAGT | 879 | C49U, | 0.15683481 |
| | TCGTTATGAACTTGAAAAAGTGGCACCGAGTCGGTGCT | | C56G | 4 |
| cr063 | ATTTTAGAGCTAAGCTGGAAACAGCATAGCAAGTTTAAATAAGGCTAGT | 880 | G0A, | 0.15646058 |
| | CCGTTATCAACTTGAAAAAGTGGCACCGAGTCGGTGCT | | A4U | 5 |
| cr221 | GTTTAAGAGCTAAGCTGGAAACAGCATAGCAAATTTAAATAAGGCTAGT | 881 | G32A, | 0.15527100 |
| | CCGTTATGAACTTGAGAAAGTGGCACCGAGTCGGTGCT | | C56G, | 1 |
| | | | A64G | |
| cr075 | GTTTCATAGCTAAGCTGGAAACAGCATAGCAAGTTTAAATAAGGCTAGT | 882 | A4C, | 0.15213573 |
| | CCGTTATCAACTTGAAAAAGTGGCACCGAGTCGGTGCT | | G6U | 5 |
| cr502 | GTTTAAGAGCTAAGCTGGAAACAGCATAGCAAGTTTAAATAAGGCTAGT | 883 | G82C | 0.14848903 |
| | CCGTTATCAACTTGAAAAAGTGGCACCGAGTCGCTGCT | | | 7 |
| cr229 | GTTTAAGAGCTAAGCTGGAAACAGCATAGCAAGTTTAAATACGGCTAGT | 884 | A41C | 0.14780650 |
| | CCGTTATCAACTTGAAAAAGTGGCACCGAGTCGGTGCT | | | 3 |
| cr228 | GTTTAAGAGCTAAGCTGGAAACAGCATAGCAAGTTTAAATAAGGGTAGT | 885 | C44G | 0.14243724 |
| | CCGTTATCAACTTGAAAAAGTGGCACCGAGTCGGTGCT | | | 2 |
| cr808 | GTTTAAGAGCTAAGCTGGAAACAGCATAGCAAGTTTAAATAAAGCTAGT | 886 | G42A, | 0.14168757 |
| | CTGTTATCAAATTGAAAAATTGTCACCGAGTCGGTGAT | | C50U, | 2 |
| | | | C59A, | |
| | | | G68U, | |
| | | | G71U, | |
| | | | C85A | |
| cr395 | GTTTAAGAGCTAAGCTGGAAACAGCATAGCAAGTTTAAATATGGCTAGT | 887 | A41U, | 0.13968530 |
| | CCGTTATCAACTTGAAAAAGTAGCACCGAATCGGTGCT | | G70A, | 8 |
| | | | G78A | |
| cr761 | GTTTAAGAGCTAAGCTGGAAACAGCATAGCTAGTTTAAATAAGGCTAGT | 888 | A30U, | 0.13692485 |
| | CCGTTGTCTACTTGAAAAAGTGGCACCGAGTCGGTGCT | | A54G, | 5 |
| | | | A57U | |
| cr881 | GTTTAAGAGCTAAGCTGGAAACAGCATAGCAAGTTTAAATAAGGCTAGT | 889 | G51U, | 0.13665256 |
| | CCTTTAACAACTTGAAAAAGTGGCACCGAGTCGGTGCT | | U55A | 9 |
| cr091 | GTTTTACAGCTAAGCTGGAAACAGCATAGCAAGTTTAAATAAGGCTAGT | 890 | A4U, | 0.13664142 |
| | CCGTTATCAACTTGAAAAAGTGGCACCGAGTCGGTGCT | | G6C | 6 |
| cr798 | GTTTAAGAGCTAAGCTGGAAACAGCATAGCAAGTTTAAATAAGGATATT | 891 | C44A, | 0.13612444 |
| | CCGTTATCAACTTGAAAAAGTGGCATCCAGTGGATGCT | | G47U, | 7 |
| | | | C74U, | |
| | | | G76C, | |
| | | | C80G, | |
| | | | G82A | |
| cr390 | GTTTAAGAGCTAAGCTGGAAACAGCATAGCAAATTTAAATAAGGCTAGT | 892 | G32A, | 0.13544846 |
| | CCGTTATCAACTTGAAAAAGTAGCACCGAGTCGGTGCT | | G70A | 3 |
| cr560 | GTTTAAGAGCTAAGCTGGAAACAGCATAGCAAGCTTAAATAAGGCTAGT | 893 | U33C, | 0.13471112 |
| | CCGTTATCAACTTGAAAAAGTGGCACTGAGTCAGTGCT | | C75U, | 5 |
| | | | G81A | |
| cr872 | GTTTAAGAGCTAAGCTGGAAACAGCATAGCAAGTTTAAATAAGGCTATT | 894 | G47U | 0.1317391 |
| | CCGTTATCAACTTGAAAAAGTGGCACCGAGTCGGTGCT | | | |
| cr490 | GTTTAAGAGCTAAGCTGGAAACAGCATAGCAAGTTTAAATAAGGCTAGT | 895 | C59A, | 0.13168726 |
| | CCGTTATCAAATTGAAAAATTGGCGCCCAGTGGGCGCT | | G68U, | 6 |
| | | | A73G, | |
| | | | G76C, | |
| | | | C80G, | |
| | | | U83C | |
| cr274 | GTTTAAGAGCTAAGCTGGAAACAGCATAGCAACTTTAAATAAGGCTAGT | 896 | G32C, | 0.13030086 |
| | CCGTTATCAACCTGAAAAGGTGGCACCGAGTCGGTGCT | | U60C, | 9 |
| | | | A67G | |
| cr259 | GTTTAAGAGCTAAGCTGGAAACAGCATAGCAAGTTTAAATAAGGCTAGT | 897 | C56G, | 0.12430892 |
| | CCGTTATGTACTTCAAAAAGTGGCACCGAGTCGGTGCT | | A57U, | 3 |
| | | | G62C | |
| cr089 | GTTTATAAGCTAAGCTGGAAACAGCATAGCAAGTTTAAATAAGGCTAGT | 898 | A5U, | 0.11827319 |
| | CCGTTATCAACTTGAAAAAGTGGCACCGAGTCGGTGCT | | G6A | 7 |
| cr461 | GTTTAAGAGCTAAGCTGGAAACAGCATAGCAAGTTTAAATAAGGCTAGT | 899 | G51U, | 0.11467393 |
| | CCTTAATCAACTTGAAGAAGTGGCACCGAGTCGGTGCT | | U53A, | 8 |
| | | | A65G | |
| cr257 | GTTTAAGAGCTAAGCTGGAAACAGCATAGCAAGTTTAAATCAGGCTAGT | 900 | A40C, | 0.11088059 |
| | CCGTTATCAACTTGAAAAAGTTGCACCGAGTCGGTGCT | | G70U | 7 |
| cr059 | GGTTAAAAGCTAAGCTGGAAACAGCATAGCAAGTTTAAATAAGGCTAGT | 901 | U1G, | 0.11059782 |
| | CCGTTATCAACTTGAAAAAGTGGCACCGAGTCGGTGCT | | G6A | 2 |
| cr538 | GTTTAAGAGCTAAGCTGGAAACAGCATAGCAAGCTTAAATAAGACTAGT | 902 | U33C, | 0.11036172 |
| | TCGTTATCAACTTGAAAAAGTGGCACCGAGTCGGTGCT | | G43A, | 5 |
| | | | C49U | |
| cr411 | GTTTAAGAGCTAAGCTGGAAACAGCATAGCAAGTTTAAATAAGACTAGT | 903 | G43A | 0.10859452 |
| | CCGTTATCAACTTGAAAAAGTGGCACCGAGTCGGTGCT | | | |
| cr580 | GTTTAAGAGCTAAGCTGGAAACAGCATAGCAAGTTTAGATAAGGCTAGT | 904 | A37G | 0.10760803 |
| | CCGTTATCAACTTGAAAAAGTGGCACCGAGTCGGTGCT | | | 5 |
| cr087 | CTTTGAGAGCTAAGCTGGAAACAGCATAGCAAGTTTAAATAAGGCTAGT | 905 | G0C, | 0.10411385 |
| | CCGTTATCAACTTGAAAAAGTGGCACCGAGTCGGTGCT | | A4G | 6 |
| cr290 | GTTTAAGAGCTAAGCTGGAAACAGCATAGCAAGTTTAAATAAGGCTAGT | 906 | G51A, | 0.10268076 |
| | CCATTATCAACTTGAAAAAGTTGCACCGAGTCGGTGCT | | G70U | 1 |
| cr288 | GTTTAAGAGCTAAGCTGGAAACAGCATAGCAACTTTAAATAAGGCTAGT | 907 | G32C, | 0.10159477 |
| | CCGTTATCAACTGGAAACAGTGGCACCGAGTCGGTGCT | | U61G, | 8 |
| | | | A66C | |
| cr062 | GTTCAAGTGCTAAGCTGGAAACAGCATAGCAAGTTTAAATAAGGCTAGT | 908 | U3C, | 0.10033929 |
| | CCGTTATCAACTTGAAAAAGTGGCACCGAGTCGGTGCT | | A7U | 1 |
| cr401 | GTTTAAGAGCTAAGCTGGAAACAGCATAGCAAGTTTAAATAAGGCTAGT | 909 | A73U, | 0.09780867 |
| | CCGTTATCAACTTGAAAAAGTGGCTCCGAGTCTGCGCT | | G81U, | 4 |
| | | | U83C | |
| cr796 | GTTTAAGAGCTAAGCTGGAAACAGCATAGCAAGTTAAAATAAGGCTAGT | 910 | U35A | 0.09600498 |
| | CCGTTATCAACTTGAAAAAGTGGCACCGAGTCGGTGCT | | | 9 |
| cr453 | GTTTAAGAGCTAAGCTGGAAACAGCATAGCAAGTTTAACTAAGGCTAGT | 911 | A38C | 0.09528018 |
| | CCGTTATCAACTTGAAAAAGTGGCACCGAGTCGGTGCT | | | 7 |
| cr513 | GTTTAAGAGCTAAGCTGGAAACAGCATAGCAACTTTAAATAAGGCTAGT | 912 | G32C, | 0.09470092 |
| | CCGTTATCACCTTGAAAAAGGGGCACCGAGTCGGTGCT | | A58C, | 7 |
| | | | U69G | |
| cr084 | GTCTGAGAGCTAAGCTGGAAACAGCATAGCAAGTTTAAATAAGGCTAGT | 913 | U2C, | 0.09372306 |
| | CCGTTATCAACTTGAAAAAGTGGCACCGAGTCGGTGCT | | A4G | 1 |
| cr112 | GTTTAAGAGCTAAGCTGGAAACAGCATAGCAAGTTTAAATAAAGTTAAT | 914 | G42A, | 0.09330089 |
| | CTGTTATCAATTTGAAAAAATGGCACCGAGTCGGTGCT | | C44U, | 7 |
| | | | G47A, | |
| | | | C50U, | |
| | | | C59U, | |
| | | | G68A | |
| cr714 | GTTTAAGAGCTAAGCTGGAAACAGCATAGCAAGCTTAAATAAGGCTAGT | 915 | U33C, | 0.09294380 |
| | CCGTTATCAAATTGAAAAATTGGCACCGAGTCGGTGCT | | C59A, | 2 |
| | | | G68U | |
| cr325 | GTTTAAGAGCTAAGCTGGAAACAGCATAGCAACTTTAAATAAGGCTAGT | 916 | G32C, | 0.09142578 |
| | CCGTTATCAGCTTGAAAAAGCGGCACCGAGTCGGTGCT | | A58G, | 5 |
| | | | U69C | |
| cr066 | ATTCAAGAGCTAAGCTGGAAACAGCATAGCAAGTTTAAATAAGGCTAGT | 917 | G0A, | 0.09114989 |
| | CCGTTATCAACTTGAAAAAGTGGCACCGAGTCGGTGCT | | U3C | 6 |
| cr762 | GTTTAAGAGCTAAGCTGGAAACAGCATAGCACGTTTAAATAAGGCTAGT | 918 | A31C, | 0.08849885 |
| | CUTTATCAACTTGAAAAAGTGGCACCGAGTCGGTGCT | | G51U | 6 |
| cr739 | GTTTAAGAGCTAAGCTGGAAACAGCATAGCAACTTTAAATAAGGCTAGT | 919 | G32C, | 0.08764186 |
| | CCGTTATCAACGTGAAAACGTGGCACCGAGTCGGTGCT | | U60G, | 1 |
| | | | A67C | |
| cr791 | GTTTAAGAGCTAAGCTGGAAACAGCATAGCAAGTTTAAATAAAGCTAGT | 920 | G42A, | 0.07648447 |
| | CTGTTATCAACTTGAAAAAGTGGCACCCAGTGGGTGCT | | C50U, | 4 |
| | | | G76C, | |
| | | | C80G | |
| cr985 | GTTTAAGAGCTAAGCTGGAAACAGCATAGCAAGTTTAAATAAGGCTAGT | 921 | G51U | 0.07561542 |
| | CUTTATCAACTTGAAAAAGTGGCACCGAGTCGGTGCT | | | 7 |
| cr588 | GTTTAAGAGCTAAGCTGGAAACAGCATAGCAACTTTAAATAAGGCTAGT | 922 | G32C, | 0.07506900 |
| | CCGTTATCAACTTGAAAAAGTGGTACCGAGTCGGTACT | | C72U, | 5 |
| | | | G84A | |
| cr082 | CGTTAAGAGCTAAGCTGGAAACAGCATAGCAAGTTTAAATAAGGCTAGT | 923 | G0C, | 0.07409889 |
| | CCGTTATCAACTTGAAAAAGTGGCACCGAGTCGGTGCT | | U1G | 7 |
| cr038 | GTTTGCGAGCTAAGCTGGAAACAGCATAGCAAGTTTAAATAAGGCTAGT | 924 | A4G, | 0.07320819 |
| | CCGTTATCAACTTGAAAAAGTGGCACCGAGTCGGTGCT | | A5C | 7 |
| cr582 | GTTTAAGAGCTAAGCTGGAAACAGCATAGCAAGTTTAAATACGGCTTGT | 925 | A41C, | 0.07314933 |
| | CCGTTATCAACTTCAAAAAGTGGCACCGAGTCGGTGCT | | A46U, | 4 |
| | | | G62C | |
| cr438 | GTTTAAGAGCTAAGCTGGAAACAGCATAGCCAGTTTAAATACGGCTAGT | 926 | A30C, | 0.06886058 |
| | CCGTTGTCAACTTGAAAAAGTGGCACCGAGTCGGTGCT | | A41C, | 8 |
| | | | A54G | |
| cr097 | GCTTACGAGCTAAGCTGGAAACAGCATAGCAAGTTTAAATAAGGCTAGT | 927 | U1C, | 0.06585236 |
| | CCGTTATCAACTTGAAAAAGTGGCACCGAGTCGGTGCT | | A5C | 4 |
| cr467 | GTTTAAGAGCTAAGCTGGAAACAGCATAGCAAGCTTAAATAAGGCTAGT | 928 | U33C, | 0.06565261 |
| | CCGTTATCAATTTGAAAAAATGGCACCGAGTCGGTGCT | | C59U, | 8 |
| | | | G68A | |
| cr197 | GTTTAAGAGCTAAGCTGGAAACAGCATAGCAAGTTTAAATAATGCTAGT | 929 | G42U, | 0.06435841 |
| | CAGTTATCAACTGGAAACAGTGGCACCGAGTCGGTGCT | | C50A, | 6 |
| | | | U61G, | |
| | | | A66C | |
| cr741 | GTTTAAGAGCTAAGCTGGAAACAGCATGGCAAGTTTAAATACGGCTAGA | 930 | A27G, | 0.06347491 |
| | CCGTTATCAACTTGAAAAAGTGGCACCGAGTCGGTGCT | | A41C, | |
| | | | U48A | |
| cr298 | GTTTAAGAGCTAAGCTGGAAACAGCATAGCAAGTTTAAATAAGGCTAGT | 931 | G51U, | 0.06335444 |
| | CUTTATCAACTTGAAAAAGTGGCACCGACTCGGTGCT | | G78C | 6 |
| cr193 | GTTTAAGAGCTAAGCTGGAAACAGCATAGCAAGTTTAAATAAAGCTAGT | 932 | G42A, | 0.06081432 |
| | CTGTTATCAATTTGAAAAAATGCCACCGAGTCGGTGGT | | C50U, | 5 |
| | | | C59U, | |
| | | | G68A, | |
| | | | G71C, | |
| | | | C85G | |
| cr514 | GTTTAAGAGCTAAGCTGGAAACAGCATAGCAACTTTAAATAAGGCTAGT | 933 | G32C, | 0.05775985 |
| | CCGTTATCAACTTGAAAAAGTGGGACCGAGTCGGTCCT | | C72G, | 1 |
| | | | G84C | |
| cr061 | GTTCAACAGCTAAGCTGGAAACAGCATAGCAAGTTTAAATAAGGCTAGT | 934 | U3C, | 0.05690570 |
| | CCGTTATCAACTTGAAAAAGTGGCACCGAGTCGGTGCT | | G6C | 1 |
| cr419 | GTTTAAGAGCTAAGCTGGAAACAGCATAGCAACTTTAAATAAGGCTAGT | 935 | G32C, | 0.05682243 |
| | CCGTTATCAACTTGAAAAAGTGGAACCGAGTCGGTTCT | | C72A, | 2 |
| | | | G84U | |
| cr928 | GTTTAAGAGCTAAGCTGGAAACAGCATAGCTAGTTTAAATGAGGCTAGT | 936 | A30U, | 0.05583216 |
| | CCGTAATCAACTTGAAAAAGTGGCACCGAGTCGGTGCT | | A40G, | |
| | | | U53A | |
| cr115 | GTTTAAGAGCTAAGCTGGAAACAGCATAGCAAGTTTAAATAATGCTAGT | 937 | G42U, | 0.05506005 |
| | CAGTTATCAACTCGAAAGAGTGGCACCGAGTCGGTGCT | | C50A, | 2 |
| | | | U61C, | |
| | | | A66G | |
| cr422 | GTTTAAGAGCTAAGCTGGAAACAGCATAGCAAGTTTAAATAAGGCTAGT | 938 | C72U, | 0.04929988 |
| | CCGTTATCAACTTGAAAAAGTGGTACCGAGTCGTTCCT | | G82U, | 7 |
| | | | G84C | |
| cr465 | GTTTAAGAGCTAAGCTGGAAACAGCATAGCAAGCTTAAATAAGGATATT | 939 | U33C, | 0.04863886 |
| | CCGTTATCAACTTGAAAAAGTGGCACCGAGTCGGTGCT | | C44A, | 7 |
| | | | G47U | |
| cr764 | GTTTAAGAGCTAAGCTGGAAACAGCATAGCAACTTTAAATAAGGCTAGT | 940 | G32C | 0.04862881 |
| | CCGTTATCAACTTGAAAAAGTGGCACCGAGTCGGTGCT | | | 7 |
| cr045 | TTTTATGAGCTAAGCTGGAAACAGCATAGCAAGTTTAAATAAGGCTAGT | 941 | G0U, | 0.04838579 |
| | CCGTTATCAACTTGAAAAAGTGGCACCGAGTCGGTGCT | | A5U | 9 |
| cr050 | CTTTAGGAGCTAAGCTGGAAACAGCATAGCAAGTTTAAATAAGGCTAGT | 942 | G0C, | 0.04784583 |
| | CCGTTATCAACTTGAAAAAGTGGCACCGAGTCGGTGCT | | A5G | 1 |
| cr677 | GTTTAAGAGCTAAGCTGGAAACAGCATAGCAAGTTTAAATTAGGCTAGT | 943 | A40U | 0.04623113 |
| | CCGTTATCAACTTGAAAAAGTGGCACCGAGTCGGTGCT | | | 4 |
| cr188 | GTTTAAGAGCTAAGCTGGAAACAGCATAGCAAGTTTAAATAATGCTAGT | 944 | G42U, | 0.04409470 |
| | CAGTTATCAACCTGAAAAGGTGGCACCGAGTCGGTGCT | | C50A, | 3 |
| | | | U60C, | |
| | | | A67G | |
| cr625 | GTTTAAGAGCTAAGCTGGAAACAGCATAGCAAGCTTAAATAAGGCTAGT | 945 | U33C, | 0.04331986 |
| | CCGTTATCAACTTGAAAAAGTGGCACCTAGTAGGTGCT | | G76U, | 9 |
| | | | C80A | |
| cr138 | GTTTAAGAGCTAAGCTGGAAACAGCATAGCAAGCTTAAATAAGGCTAGT | 946 | U33C, | 0.04306703 |
| | CCGTTATCAACTTGAAAAAGTGGCACGGAGTCCGTGCT | | C75G, | 4 |
| | | | G81C | |
| cr211 | GTTTAAGAGCTAAGCTGGAAACAGCATAGCAAGTTTAAATAAGTCTAGT | 947 | G43U | 0.04074650 |
| | CCGTTATCAACTTGAAAAAGTGGCACCGAGTCGGTGCT | | | 7 |
| cr708 | GTTTAAGAGCTAAGCTGGAAACAGCATAGCAAGTTTAAATAATGCTAGT | 948 | G42U, | 0.03939779 |
| | CAGTTATCAACTTGAAAAAGTGGCACCGAGTCGGTGCT | | C50A | 7 |
| cr222 | GTTTAAGAGCTAAGCTGGAAACAGCATAGCAAGGTTAAATAAGGCTAGT | 949 | U33G, | 0.03915274 |
| | CCGTTATCAGCTTGAAAAAGCGGCACCGAGTCGGTGCT | | A58G, | 9 |
| | | | U69C | |
| cr107 | GTTTAAGAGCTAAGCTGGAAACAGCATAGCAAGTTTAAATAATGCTAGT | 950 | G42U, | 0.03837022 |
| | CAGTTATCAACTTGAAAAAGTGGCAACGAGTCGTTGCT | | C50A, | 2 |
| | | | C74A, | |
| | | | G82U | |
| cr767 | GTTTAAGAGCTAAGCTGGAAACAGCATAGCAAGATTAAATAAGGCTAGT | 951 | U33A, | 0.03810067 |
| | CCGTTATCAACTTGAAAAAGTGGCACCGTGTCGGTGCT | | A77U | 3 |
| cr208 | GTTTAAGAGCTAAGCTGGAAACAGCATAGCAAGTTTAAATAAGGCTAGT | 952 | G51U, | 0.03710978 |
| | CCTTCATCAACTTGAAGAAGTGGCACCGAGTCGGTGCT | | U53C, | 1 |
| | | | A65G | |
| cr498 | GTTTAAGAGCTAAGCTGGAAACAGCATAGCAAGTTTAATTAAGGCTAGT | 953 | A38U | 0.03563223 |
| | CCGTTATCAACTTGAAAAAGTGGCACCGAGTCGGTGCT | | | 8 |
| cr681 | GTTTAAGAGCTAAGCTGGAAACAGCATAGCAAGTTTAAATAAGGCTACT | 954 | G47C, | 0.03559186 |
| | CCGTTATTAACTTGAAAAAGTGGCACCGAGTCGGTGCT | | C56U | |
| cr078 | GTTTTGGAGCTAAGCTGGAAACAGCATAGCAAGTTTAAATAAGGCTAGT | 955 | A4U, | 0.03506607 |
| | CCGTTATCAACTTGAAAAAGTGGCACCGAGTCGGTGCT | | A5G | 4 |
| cr743 | GTTTAAGAGCTAAGCTGGAAACAGCATAGCAACTTTAAATAAGGCTAGT | 956 | G32C, | 0.03495136 |
| | CCGTTATCAACTTGAAAAAGTGGCGCCGAGTCGGCGCT | | A73G, | 2 |
| | | | U83C | |
| cr202 | GTTTAAGAGCTAAGCTGGAAACAGCATAGCAACTTTAAATAAGGCTAGT | 957 | G32C, | 0.03423087 |
| | CCGTTATCAACTTGAAAAAGTGACACCGAGTCGGTGTT | | G71A, | 5 |
| | | | C85U | |
| cr526 | GTTTAAGAGCTAAGCTGGAAACAGCATAGCAAGTTTAAATAATGCTAGT | 958 | G42U, | 0.03359079 |
| | CAGTTATCAACTTGAAAAAGTGGCGCCGAGTCGGCGCT | | C50A, | |
| | | | A73G, | |
| | | | U83C | |
| cr738 | GTTTAAGAGCTAAGCTGGAAACAGCATAGCATGTTTAAATTAGGCTAGT | 959 | A31U, | 0.03305876 |
| | CCGTTATCAACTTGAAAAAGTGGCACCGAGTCGGTGCT | | A40U | 8 |
| cr241 | GTTTAAGAGCTAAGCTGGAAACAGCATAGCAACTTTAAATAAGGCTAGT | 960 | G32C, | 0.03304388 |
| | CCGTTATCAACATGAAAATUGGCACCGAGTCGGTGCT | | U60A, | 9 |
| | | | A67U | |
| cr314 | GTTTAAGAGCTAAGCTGGAAACAGCATAGCAAGGTTAAATAAGGCTAGT | 961 | U33G, | 0.03303268 |
| | CCGTTATCAACTTGAAAAAGTGGAACCGAGTCGGTTCT | | C72A, | |
| | | | G84U | |
| cr007 | GTATAAGAGCTAAGCTGGAAACAGCATAGCAAGTTTAAATAAGGCTAGT | 962 | U2A | 0.03285098 |
| | CCGTTATCAACTTGAAAAAGTGGCACCGAGTCGGTGCT | | | 2 |
| cr357 | GTTTAAGAGCTAAGCTGGAAACAGCATAGCAAGGTTAAATAAGGCTAGT | 963 | U33G, | 0.03252715 |
| | CCGTTATCAACTTGAAAAAGTGGCTCCGAGTCGGAGCT | | A73U, | 9 |
| | | | U83A | |
| cr574 | GTTTAAGAGCTAAGCTGGAAACAGCATAGCAAGTTTCAATAAGGCTAGT | 964 | A36C | 0.03217055 |
| | CCGTTATCAACTTGAAAAAGTGGCACCGAGTCGGTGCT | | | 5 |
| cr313 | GTTTAAGAGCTAAGCTGGAAACAGCATAGCAAGTTTAAATAAGGCTAGT | 965 | C72G, | 0.03202363 |
| | CCGTTATCAACTTGAAAAAGTGGGTGGGAGTCGCTCCT | | A73U, | 4 |
| | | | C74G, | |
| | | | C75G, | |
| | | | G82C, | |
| | | | G84C | |
| cr571 | GTTTAAGAGCTAAGCTGGAAACAGCATAGCAAGGTTAAATAAGGCTAGT | 966 | U33G, | 0.03194090 |
| | CCGTTATCAACTCGAAAGAGTGGCACCGAGTCGGTGCT | | U61C, | 9 |
| | | | A66G | |
| cr151 | GTTTAAGAGCTAAGCTGGAAACAGCATAGCAACTTTAAATAAGGCTAGT | 967 | G32C, | 0.03186099 |
| | CCGGTATCAACTTGAAAAAGTGGCACCGAGTCGGTGCT | | U52G | 3 |
| cr880 | GTTTAAGAGCTAAGCTGGAAACAGCATAGCAAGGTTAAATAAGGCTAGT | 968 | U33G, | 0.03182692 |
| | CCGTTATCAACTTGAAAAAGTGGCATCGAGTCGATGCT | | C74U, | |
| | | | G82A | |
| cr301 | GTTTAAGAGCTAAGCTGGAAACAGCATAGCAACTTTAAATAAGGCTAGT | 969 | G32C, | 0.03131975 |
| | CCGTTATCAACTTGAAAAAGTGGCACCGATTCGGTGCT | | G78U | 2 |
| cr988 | GTTTAAGAGCTAAGCTGGAAACAGCATAGCAACTTTAAATAAGGCTAGT | 970 | G32C, | 0.03049523 |
| | CCGTTATCAAGTTGAAAAACTGGCACCGAGTCGGTGCT | | C59G, | 8 |
| | | | G68C | |
| cr716 | GTTTAAGAGCTAAGCTGGAAACAGCATAGCCATTTTAAATAAGGCTAGT | 971 | A30C, | 0.03032557 |
| | CCGTTATCAACTTGAAAAAGTGGCACCGAGTCGGTGCT | | G32U | 9 |
| cr312 | GTTTAAGAGCTAAGCTGGAAACAGCATAGCAAGGTTAAATAAGGCTAGT | 972 | U33G, | 0.03032324 |
| | CCGTTATCAACGTGAAAACGTGGCACCGAGTCGGTGCT | | U60G, | 7 |
| | | | A67C | |
| cr638 | GTTTAAGAGCTAAGCTGGAAACAGCATAGCAAGCTTAAATAAGGGTACT | 973 | U33C, | 0.03011832 |
| | CCGTTATCAACTTGAAAAAGTGGCACCGAGTCGGTGCT | | C44G, | 1 |
| | | | G47C | |
| cr010 | GTTAAAGAGCTAAGCTGGAAACAGCATAGCAAGTTTAAATAAGGCTAGT | 974 | U3A | 0.02975655 |
| | CCGTTATCAACTTGAAAAAGTGGCACCGAGTCGGTGCT | | | 2 |
| cr095 | GTATAAGTGCTAAGCTGGAAACAGCATAGCAAGTTTAAATAAGGCTAGT | 975 | U2A, | 0.0297361 |
| | CCGTTATCAACTTGAAAAAGTGGCACCGAGTCGGTGCT | | A7U | |
| cr830 | GTTTAAGAGCTAAGCTGGAAACAGCATAGCAAGTTTAAATAAGGCTAGG | 976 | U48G, | 0.02925330 |
| | CCGTTATCAACTTGAAAAAGTGTCACCGAGTCGGTGCT | | G71U | 4 |
| cr378 | GTTTAAGAGCTAAGCTGGAAACAGCATAGCAAGTTTAAATGAGGCTAGT | 977 | A40G | 0.02892768 |
| | CCGTTATCAACTTGAAAAAGTGGCACCGAGTCGGTGCT | | | |
| cr933 | GTTTAAGAGCTAAGCTGGAAACAGCATAGCAAGGTTAAATAAGGCTAGT | 978 | U33G, | 0.02860249 |
| | CCGTTATCAACTTGAAAAAGTGTCACCGAGTCGGTGAT | | G71U, | 3 |
| | | | C85A | |
| cr757 | GTTTAAGAGCTAAGCTGGAAACAGCATAGCAACTTTAAATAAGGCTAGT | 979 | G32C, | 0.02850858 |
| | CCGTTATCAACTAGAAATAGTGGCACCGAGTCGGTGCT | | U61A, | 4 |
| | | | A66U | |
| cr570 | GTTTAAGAGCTAAGCTGGAAACAGCATAGCAACTTTAAATAAGGCTAGT | 980 | G32C, | 0.02846076 |
| | CCGTTATCAACTTGAAAAAGTGGCTCCGAGTCGGAGCT | | A73U, | 9 |
| | | | U83A | |
| cr009 | GTGTAAGAGCTAAGCTGGAAACAGCATAGCAAGTTTAAATAAGGCTAGT | 981 | U2G | 0.02782799 |
| | CCGTTATCAACTTGAAAAAGTGGCACCGAGTCGGTGCT | | | 7 |
| cr012 | GTTGAAGAGCTAAGCTGGAAACAGCATAGCAAGTTTAAATAAGGCTAGT | 982 | U3G | 0.02768842 |
| | CCGTTATCAACTTGAAAAAGTGGCACCGAGTCGGTGCT | | | 7 |
| cr096 | GTTTGTGAGCTAAGCTGGAAACAGCATAGCAAGTTTAAATAAGGCTAGT | 983 | A4G, | 0.02763834 |
| | CCGTTATCAACTTGAAAAAGTGGCACCGAGTCGGTGCT | | A5U | 2 |
| cr627 | GTTTAAGAGCTAAGCTGGAAACAGCATAGCAAGTTTAAATAATGCTAGT | 984 | G42U, | 0.02763056 |
| | CAGTTATCAACTTGAGAAAGTGGCACCGAGTCGGTGCT | | C50A, | 2 |
| | | | A64G | |
| cr048 | TTTCAAGAGCTAAGCTGGAAACAGCATAGCAAGTTTAAATAAGGCTAGT | 985 | G0U, | 0.02761154 |
| | CCGTTATCAACTTGAAAAAGTGGCACCGAGTCGGTGCT | | U3C | 7 |
| cr641 | GTTTAAGAGCTAAGCTGGAAACAGCATAGCAAGGTTAAATAAGGCTAGT | 986 | U33G | 0.02687199 |
| | CCGTTATCAACTTGAAAAAGTGGCACCGAGTCGGTGCT | | | 8 |
| cr711 | GTTTAAGAGCTAAGCTGGAAACAGCATAGCAAGGTTAAATAAGGCTAGT | 987 | U33G, | 0.02669595 |
| | CCGTTATCAACTTGAAAAAGTGCCACCGAGTCGGTGGT | | G71C, | 1 |
| | | | C85G | |
| cr085 | GTTTTTGAGCTAAGCTGGAAACAGCATAGCAAGTTTAAATAAGGCTAGT | 988 | A4U, | 0.02665626 |
| | CCGTTATCAACTTGAAAAAGTGGCACCGAGTCGGTGCT | | A5U | 6 |
| cr032 | GATTGAGAGCTAAGCTGGAAACAGCATAGCAAGTTTAAATAAGGCTAGT | 989 | U1A, | 0.02645980 |
| | CCGTTATCAACTTGAAAAAGTGGCACCGAGTCGGTGCT | | A4G | 2 |
| cr745 | GTTTAAGAGCTAAGCTGGAAACAGCATAGCAAGTTTAAATAAAGCTAGT | 990 | G42A | 0.02630658 |
| | CCGTTATCAACTTGAAAAAGTGGCACCGAGTCGGTGCT | | | 3 |
| cr952 | GTTTAAGAGCTAAGCTGGAAACAGCATAGCTAGTTTAAATAAGGCTAGG | 991 | A30U, | 0.02620670 |
| | CCGTAATCAACTTGAAAAAGTGGCACCGAGTCGGTGCT | | U48G, | 9 |
| | | | U53A | |
| cr454 | GTTTAAGAGCTAAGCTGGAAACAGCATAGCAAGTTTAAATAATGCTAGT | 992 | G42U, | 0.02618139 |
| | CAGTTATCACCTTGAAAAAGGGGTACCGAGTCGGTACT | | C50A, | 5 |
| | | | A58C, | |
| | | | U69G, | |
| | | | C72U, | |
| | | | G84A | |
| cr986 | GTTTAAGAGCTAAGCTGGAAACAGCATAGCAAGTTTAAATAAGGCTAGG | 993 | U48G | 0.02579911 |
| | CCGTTATCAACTTGAAAAAGTGGCACCGAGTCGGTGCT | | | 4 |
| cr737 | GTTTAAGAGCTAAGCTGGAAACAGCATAGCAAGTTTAAATAAGGCTAGT | 994 | C74G, | 0.02539394 |
| | CCGTTATCAACTTGAAAAAGTGGCAGGCTGTGGCTGCT | | C75G, | 1 |
| | | | G76C, | |
| | | | A77U, | |
| | | | C80G, | |
| | | | G82C | |
| cr958 | GTTTAAGAGCTAAGCTGGAAACAGCATAGCAACTTTAAATAAGGCTAGT | 995 | G32C, | 0.02490160 |
| | CCGTTATCAACTTGAAAAAGTGGCACTGAGTCAGTGCT | | C75U, | 2 |
| | | | G81A | |
| cr581 | GTTTAAGAGCTAAGCTGGAAACAGCATAGCAAGTTTTAATAAGGCTAGT | 996 | A36U | 0.02487978 |
| | CCGTTATCAACTTGAAAAAGTGGCACCGAGTCGGTGCT | | | 3 |
| cr043 | GGTTTAGAGCTAAGCTGGAAACAGCATAGCAAGTTTAAATAAGGCTAGT | 997 | U1G, | 0.02450376 |
| | CCGTTATCAACTTGAAAAAGTGGCACCGAGTCGGTGCT | | A4U | |
| cr633 | GTTTAAGAGCTAAGCTGGAAACAGCATGGCAATTTTAAATACGGCTAGT | 998 | A27G, | 0.02444926 |
| | CCGTTATCAACTTGAAAAAGTGGCACCGAGTCGGTGCT | | G32U, | 8 |
| | | | A41C | |
| cr386 | GTTTAAGAGCTAAGCTGGAAACAGCATAGCAAGGTTAAATAAGGCTAGT | 999 | U33G, | 0.02427673 |
| | CCGTTATCAACCTGAAAAGGTGGCACCGAGTCGGTGCT | | U60C, | 3 |
| | | | A67G | |
| cr935 | GTTTAAGAGCTAAGCTGGAAACAGCATAGCAAGTTTAAATGAGGCTAGT | 1000 | A40G, | 0.02412571 |
| | CCGTTATCAACTTGACAAAGTGGCACCGAGTCGGTGCT | | A64C | 5 |
| cr946 | GTTTAAGAGCTAAGCTGGAAACAGCATAGCAAGTTTAAATAACGCTAGT | 1001 | G42C, | 0.02369839 |
| | CGGTTATCAACTGGAAACAGTGGCGCCGAGTCGGCGCT | | C50G, | 8 |
| | | | U61G, | |
| | | | A66C, | |
| | | | A73G, | |
| | | | U83C | |
| cr922 | GTTTAAGAGCTAAGCTGGAAACAGCATAGCAAGGTTAAATAAGGCTAGT | 1002 | U33G, | 0.02327413 |
| | CCGTTATCAACTTGAAAAAGTGGCAACGAGTCGTTGCT | | C74A, | 3 |
| | | | G82U | |
| cr080 | TTTTTAGAGCTAAGCTGGAAACAGCATAGCAAGTTTAAATAAGGCTAGT | 1003 | G0U, | 0.02322959 |
| | CCGTTATCAACTTGAAAAAGTGGCACCGAGTCGGTGCT | | A4U | 3 |
| cr950 | GTTTAAGAGCTAAGCTGGAAACAGCATAGCTAGGTTAAATAAGGCTAGT | 1004 | A30U, | 0.02240303 |
| | CCGTGATCAACTTGAAAAAGTGGCACCGAGTCGGTGCT | | U33G, | 7 |
| | | | U53G | |
| cr547 | GTTTAAGAGCTAAGCTGGAAACAGCATAGCAAGTTTAAATGAGGCTAGT | 1005 | A40G, | 0.02193573 |
| | CCGTTATCAACTTGAAAAAGTGGCACCGAATCGGTGCT | | G78A | 2 |
| cr542 | GTTTAAGAGCTAAGCTGGAAACAGCATAGCAAGATTAAATAAGGCTAGT | 1006 | U33A, | 0.02115297 |
| | CCGTCATCGACTTGAAAAAGTGGCACCGAGTCGGTGCT | | U53C, | 5 |
| | | | A57G | |
| cr700 | GTTTAAGAGCTAAGCTGGAAACAGCATAGCAAGTTTAAATTAGGCTAGT | 1007 | A40U, | 0.02102791 |
| | CCGTTATCAACTTGAAAAAGTGGCACCGCGTCGGTGCT | | A77C | 1 |
| cr030 | GCTTTAGAGCTAAGCTGGAAACAGCATAGCAAGTTTAAATAAGGCTAGT | 1008 | U1C, | 0.02089383 |
| | CCGTTATCAACTTGAAAAAGTGGCACCGAGTCGGTGCT | | A4U | 7 |
| cr680 | GTTTAAGAGCTAAGCTGGAAACAGCATAGCAAGTTTAAATAACGCTAGT | 1009 | G42C, | 0.02085932 |
| | CGGTTATCAACATGAAAATGTGGCTCCGAGTCGGAGCT | | C50G, | 1 |
| | | | U60A, | |
| | | | A67U, | |
| | | | A73U, | |
| | | | U83A | |
| cr496 | GTTTAAGAGCTAAGCTGGAAACAGCATAACAAGTTTAAATAAGGCTAGT | 1010 | G28A, | 0.02078354 |
| | CCTTTATCAACTTGAAAAAGTGGCACCGAGACGGTGCT | | G51U, | 8 |
| | | | U79A | |
| cr305 | GTTTAAGAGCTAAGCTGGAAACAGCATAGCAAGGTTAAATAAGGCTAGT | 1011 | U33G, | 0.02073443 |
| | CCGTTATCAACTTGAAAAAGTGGCCCCGAGTCGGGGCT | | A73C, | 3 |
| | | | U83G | |
| cr418 | GTTTAAGAGCTAAGCTGGAAACAGCATAGCAAGGTTAAATAAGGCTAGT | 1012 | U33G, | 0.02069461 |
| | CCGTTATCAACATGAAAATGTGGCACCGAGTCGGTGCT | | U60A, | 5 |
| | | | A67U | |
| cr186 | GTTTAAGAGCTAAGCTGGAAACAGCATAGCAAGGTTAAATAAGGCTAGT | 1013 | U33G, | 0.01990264 |
| | CCGTTATCAAGTTGAAAAACTGGCACCGAGTCGGTGCT | | C59G, | 4 |
| | | | G68C | |
| cr507 | GTTTAAGAGCTAAGCTGGAAACAGCATAGCAAGTTTAAATAATGCTAGT | 1014 | G42U, | 0.01953843 |
| | CAGTTATCAACTTGAAAAAGTGGCACAGAGTCTGTGCT | | C50A, | 8 |
| | | | C75A, | |
| | | | G81U | |
| cr389 | GTTTAAGAGCTAAGCTGGAAACAGCATAGCAAGTTTAAATAAAACTAGT | 1015 | G42A, | 0.01943923 |
| | TTGTTATCAACTTGAAAAAGTGGCACCGAGTCGGTGCT | | G43A, | 1 |
| | | | C49U, | |
| | | | C50U | |
| cr519 | GTTTAAGAGCTAAGCTGGAAACAGCATAGCAAGGTTAAATAAGGCTAGT | 1016 | U33G, | 0.01932892 |
| | CCGTTATCAACTTGAAAAAGTGGCGCCGAGTCGGCGCT | | A73G, | 7 |
| | | | U83C | |
| cr834 | GTTTAAGAGCTAAGCTGGAAACAGCATAGCAAGGTTAAATAAGGCTAGT | 1017 | U33G, | 0.01861355 |
| | CCGTTATCAACTTGAGAAAGTGGCACCGAGTCGGTGCT | | A64G | 7 |
| cr541 | GTTTAAGAGCTAAGCTGGAAACAGCATAGCAAGTTTAAATAAAGGTACT | 1018 | G42A, | 0.01856181 |
| | CTGTTATCAACTTGAAAAAGTGGTACCGAGTCGGTACT | | C44G, | 3 |
| | | | G47C, | |
| | | | C50U, | |
| | | | C72U, | |
| | | | G84A | |
| cr987 | GTTTAAGAGCTAAGCTGGAAACAGCATAGCAAGTTTAAATAAGGCTAGG | 1019 | U48G, | 0.01814568 |
| | CCGTTATCAACTTGATAAAGTGGCACCGAGTCGGTGCT | | A64U | 1 |
| cr954 | GTTTAAGAGCTAAGCTGGAAACAGCATAGCAAGGTTAAATAAGGCTAGT | 1020 | U33G, | 0.01751493 |
| | CCGTTATCAAATTGAAAAATTGGCACCGAGTCGGTGCT | | C59A, | 9 |
| | | | G68U | |
| cr448 | GTTTAAGAGCTAAGCTGGAAACAGCATAGCAACTTTAAATAAGCCTAGT | 1021 | G32C, | 0.01746477 |
| | GCGTTATCAACTTGAAAAAGTGGCACCGAGTCGGTGCT | | G43C, | |
| | | | C49G | |
| cr382 | GTTTAAGAGCTAAGCTGGAAACAGCATAGCAAGGTTAAATAACGCTAGT | 1022 | U33G, | 0.01714583 |
| | CGGTTATCAACTTGAAAAAGTGGCACCGAGTCGGTGCT | | G42C, | 5 |
| | | | C50G | |
| cr324 | GTTTAAGAGCTAAGCTGGAAACAGCATAGCAAGTTTAAATAACGCTAGT | 1023 | G42C, | 0.01711128 |
| | CGGTTATCAACCTGAAAAGGTGGCACCGAGTCGGTGCT | | C50G, | 5 |
| | | | U60C, | |
| | | | A67G | |
| cr504 | GTTTAAGAGCTAAGCTGGAAACAGCATAGCAAGGTTAAATAAGGCTAGT | 1024 | U33G, | 0.01684638 |
| | CCGTTATCAACTGGAAACAGTGGCACCGAGTCGGTGCT | | U61G, | 4 |
| | | | A66C | |
| cr864 | GTTTAAGAGCTAAGCTGGAAACAGCATAACAAGTTTAAATTAGGCTAGT | 1025 | G28A, | 0.01677620 |
| | CCGTTATCAACTTGAAAAAGTGGCACCGAGTCGGTGCT | | A40U | 7 |
| cr031 | GTAAAAGAGCTAAGCTGGAAACAGCATAGCAAGTTTAAATAAGGCTAGT | 1026 | U2A, | 0.01676682 |
| | CCGTTATCAACTTGAAAAAGTGGCACCGAGTCGGTGCT | | U3A | 2 |
| cr042 | GTGGAAGAGCTAAGCTGGAAACAGCATAGCAAGTTTAAATAAGGCTAGT | 1027 | U2G, | 0.01640069 |
| | CCGTTATCAACTTGAAAAAGTGGCACCGAGTCGGTGCT | | U3G | 1 |
| cr841 | GTTTAAGAGCTAAGCTGGAAACAGCATAGCAAGGTTAAATAAGGCTAGT | 1028 | U33G, | 0.01635699 |
| | CCGTTATCACCTTGAAAAAGGGGCACCGAGTCGGTGCT | | A58C, | 1 |
| | | | U69G | |
| cr336 | GTTTAAGAGCTAAGCTGGAAACAGCATAGCAAGTTTAAATAAGGGTACT | 1029 | C44G, | 0.01615738 |
| | CCGTTATCAAATTGAAAAATTGGCACCCAGTGGGTGCT | | G47C, | 6 |
| | | | C59A, | |
| | | | G68U, | |
| | | | G76C, | |
| | | | C80G | |
| cr963 | GTTTAAGAGCTAAGCTGGAAACAGCATAGCAAGTTTAAATAACGCTAGT | 1030 | G42C, | 0.01550548 |
| | CGGTTATCAACTTGAAAAAGTGGCGCTGAGTCAGCGCT | | C50G, | 6 |
| | | | A73G, | |
| | | | C75U, | |
| | | | G81A, | |
| | | | U83C | |
| cr731 | GTTTAAGAGCTAAGCTGGAAACAGCATAGCAAGGTTAAATAAGGCTAGT | 1031 | U33G, | 0.01504990 |
| | CCGTTATCAACTTGAAAAAGTGGTACCGAGTCGGTACT | | C72U, | 5 |
| | | | G84A | |
| cr170 | GTTTAAGAGCTAAGCTGGAAACAGCATAGCAAGGTTAAATAAGGCTAGT | 1032 | U33G, | 0.01500767 |
| | CCGTTATCAACTTGAAAAAGTGACACCGAGTCGGTGTT | | G71A, | 1 |
| | | | C85U | |
| cr462 | GTTTAAGAGCTAAGCTGGAAACAGCATAGCAAAATTAAATAAGGCTAGT | 1033 | G32A, | 0.01488192 |
| | CCGTTATCAACTTGAAAAAGTGGCACCGAGTCGGTGCT | | U33A | 9 |
| cr261 | GTTTAAGAGCTAAGCTGGAAACAGCATAGCAAGGTTAAATAAGGCTAGT | 1034 | U33G, | 0.01441044 |
| | CCGGTATCAACTTGAAAAAGTGGCACCGAGTCGGTGCT | | U52G | 1 |
| cr384 | GTTTAAGAGCTAAGCTGGAAACAGCATAGCAAGTTTAAATAAATCTAGT | 1035 | G42A, | 0.01438038 |
| | ATGTTATCAACTTGAAAAAGTGGCACCGAGTCGGTGCT | | G43U, | 3 |
| | | | C49A, | |
| | | | C50U | |
| cr413 | GTTTAAGAGCTAAGCTGGAAACAGCATAGCAACTTTAAATAAGGCTAGT | 1036 | G32C, | 0.01426502 |
| | CCGTTATCAACTTGAAAAAGTGGCAACGAGTCGTTGCT | | C74A, | 4 |
| | | | G82U | |
| cr316 | GTTTAAGAGCTAAGCTGGAAACAGCATAGCAAGTTTAAATAATGCTAGT | 1037 | G42U, | 0.01421508 |
| | CAGTTATCAACTTGAAAAAGTGGCACCAAGTTGGTGCT | | C50A, | 7 |
| | | | G76A, | |
| | | | C80U | |
| cr041 | GCTTCAGAGCTAAGCTGGAAACAGCATAGCAAGTTTAAATAAGGCTAGT | 1038 | U1C, | 0.01415250 |
| | CCGTTATCAACTTGAAAAAGTGGCACCGAGTCGGTGCT | | A4C | 7 |
| cr562 | GTTTAAGAGCTAAGCTGGAAACAGCATAGCGACTTTAAATAAGGCTAGT | 1039 | A30G, | 0.01393306 |
| | CCGTTATCAACTTGAGAAAGTGGCACCGAGTCGGTGCT | | G32C, | 6 |
| | | | A64G | |
| cr157 | GTTTAAGAGCTAAGCTGGAAACAGCATAGCAACTTTAAATAAGGCTAGT | 1040 | G32C, | 0.01383452 |
| | CCGTTATCAACTTGAAAAAGTGGCATCGAGTCGATGCT | | C74U, | |
| | | | G82A | |
| cr028 | GTCCAAGAGCTAAGCTGGAAACAGCATAGCAAGTTTAAATAAGGCTAGT | 1041 | U2C, | 0.01375137 |
| | CCGTTATCAACTTGAAAAAGTGGCACCGAGTCGGTGCT | | U3C | 1 |
| cr248 | GTTTAAGAGCTAAGCTGGAAACAGCATAGCAAGTTTAAATAACGCTAGT | 1042 | G42C, | 0.01368982 |
| | CGGTTATCAACTTGAAAAAGTGGCACCGAGTCGGTGCT | | C50G | 2 |
| cr310 | GTTTAAGAGCTAAGCTGGAAACAGCATAGCAAGTTTAAATTAGGCTAGT | 1043 | A40U, | 0.01362721 |
| | CCGTTATCAACTTGAATAAGTGGCACCGAGTCGGTGCT | | A65U | 9 |
| cr191 | GTTTAAGAGCTAAGCTGGAAACAGCATAGCAACTTTAAATAAGGCTAGT | 1044 | G32C, | 0.01357575 |
| | CCGTTATCAACTTGAAAAAGTGGCAGCGAGTCGCTGCT | | C74G, | 8 |
| | | | G82C | |
| cr773 | GTTTAAGAGCTAAGCTGGAAACAGCATAGCAAGGTTAAATAAGGCTTGT | 1045 | U33G, | 0.01327276 |
| | CCGTTATCAACTTGGAAAAGTGGCACCGAGTCGGTGCT | | A46U, | |
| | | | A63G | |
| cr424 | GTTTAAGAGCTAAGCTGGAAACAGCATAGCAAGTTTAAATAACGCTAGT | 1046 | G42C, | 0.01318765 |
| | CGGTTATCAACTTGAAAAAGTGGCGTCGAGTCGACGCT | | C50G, | 7 |
| | | | A73G, | |
| | | | C74U, | |
| | | | G82A, | |
| | | | U83C | |
| cr337 | GTTTAAGAGCTAAGCTGGAAACAGCATAGCAAGTTTAAATAACGCTAGT | 1047 | G42C, | 0.01313069 |
| | CGGTTATCAACTTGAGAAAGTGGCACCGAGTCGGTGCT | | C50G, | 4 |
| | | | A64G | |
| cr111 | GTTTAAGAGCTAAGCTGGAAACAGCATAGCAACTTTAAATAAGGCTAGT | 1048 | G32C, | 0.01299682 |
| | CCGTTATCAACTTGAAAAAGTGGCACAGAGTCTGTGCT | | C75A, | 4 |
| | | | G81U | |
| cr665 | GTTTAAGAGCTAAGCTGGAAACAGCATAGCAACTTTAAATAAGGCTAGT | 1049 | G32C, | 0.01293633 |
| | CCGTTATCAACTTGAAAAAGTGCCACCGAGTCGGTGGT | | G71C, | 7 |
| | | | C85G | |
| cr280 | GTTTAAGAGCTAAGCTGGAAACAGCATAGCAACTTTAAATAAGGCTAGT | 1050 | G32C, | 0.01272726 |
| | CCGTTATCAACTTGAAAAAGTGTCACCGAGTCGGTGAT | | G71U, | 2 |
| | | | C85A | |
| cr103 | GTTTAAGAGCTAAGCTGGAAACAGCATAGCATGTTTAAATAAGGCTAGT | 1051 | A31U, | 0.01258621 |
| | CCTTTATCAACTTGAAAAAGTGGCACCGAGGCGGTGCT | | G51U, | 1 |
| | | | U79G | |
| cr528 | GTTTAAGAGCTAAGCTGGAAACAGCATAGCCGCTTTAAATAAGGCTAGT | 1052 | A30C, | 0.01243089 |
| | CCGTTATCAACTTGAAAAAGTGGCACCGAGTCGGTGCT | | A31G, | 8 |
| | | | G32C | |
| cr204 | GTTTAAGAGCTAAGCTGGAAACAGCATAGCAAGCTTAAATAAGTCTAGT | 1053 | U33C, | 0.01236156 |
| | ACGTTATCAACTTGAAAAAGTGGCACCGAGTCGGTGCT | | G43U, | 9 |
| | | | C49A | |
| cr079 | GGTCAAGAGCTAAGCTGGAAACAGCATAGCAAGTTTAAATAAGGCTAGT | 1054 | U1G, | 0.01223007 |
| | CCGTTATCAACTTGAAAAAGTGGCACCGAGTCGGTGCT | | U3C | 3 |
| cr024 | TTGTAAGAGCTAAGCTGGAAACAGCATAGCAAGTTTAAATAAGGCTAGT | 1055 | G0U, | 0.01205485 |
| | CCGTTATCAACTTGAAAAAGTGGCACCGAGTCGGTGCT | | U2G | 5 |
| cr268 | GTTTAAGAGCTAAGCTGGAAACAGCATAGCAAGGTTAAATAAGGCTAGT | 1056 | U33G, | 0.01165832 |
| | CCGTTATCAACTTGAAAAAGTGGCACAGAGTCTGTGCT | | C75A, | 3 |
| | | | G81U | |
| cr332 | GTTTAAGAGCTAAGCTGGAAACAGCATAGCAATATTAAATAAGGCTAGT | 1057 | G32U, | 0.01144860 |
| | CCGTTATCAACTTGAAAAAGTGGCACCGAGTCGGTGCT | | U33A | 8 |
| cr649 | GTTTAAGAGCTAAGCTGGAAACAGCATAGCAACTTTAAATAAGGTTAAT | 1058 | G32C, | 0.01138431 |
| | CCGTTATCAACTTGAAAAAGTGGCACCGAGTCGGTGCT | | C44U, | 8 |
| | | | G47A | |
| cr475 | GTTTAAGAGCTAAGCTGGAAACAGCATAGCAAGTTTAAATTAGGCTAGT | 1059 | A40U, | 0.01129852 |
| | CCGTTATCAACTTGATAAAGTGGCACCGAGTCGGTGCT | | A64U | 9 |
| cr613 | GTTTAAGAGCTAAGCTGGAAACAGCATAGCAAGTTTAAATAACGGTACT | 1060 | G42C, | 0.01125210 |
| | CGGTTATCAACTTGAAAAAGTGGCACCGAGTCGGTGCT | | C44G, | 5 |
| | | | G47C, | |
| | | | C50G | |
| cr750 | GTTTAAGAGCTAAGCTGGAAACAGCATAGCAAGGTTAAATAAGGCTAGT | 1061 | U33G, | 0.01124813 |
| | CCGTTATCAATTTGAAAAAATGGCACCGAGTCGGTGCT | | C59U, | 8 |
| | | | G68A | |
| cr663 | GTTTAAGAGCTAAGCTGGAAACAGCATAGCAAGGTTAAATAAGGCTAGT | 1062 | U33G, | 0.01081332 |
| | CCGTTATCAACTTGAAAAAGTGGCACCTAGTAGGTGCT | | G76U, | 9 |
| | | | C80A | |
| cr445 | GTTTAAGAGCTAAGCTGGAAACAGCATAGCAAGTTTAAATAAGGCTAGT | 1063 | C74U, | 0.01078478 |
| | CCGTTATCAACTTGAAAAAGTGGCATCCAGTTGCTGCT | | G76C, | 5 |
| | | | C80U, | |
| | | | G82C | |
| cr509 | GTTTAAGAGCTAAGCTGGAAACAGCATAGCAAGTTTAAATAACGCTAGT | 1064 | G42C, | 0.01060821 |
| | CGGTTATCAACTTGAAAAAGTGGCAACGAGTCGTTGCT | | C50G, | 2 |
| | | | C74A, | |
| | | | G82U | |
| cr044 | GTGTAAGTGCTAAGCTGGAAACAGCATAGCAAGTTTAAATAAGGCTAGT | 1065 | U2G, | 0.01046099 |
| | CCGTTATCAACTTGAAAAAGTGGCACCGAGTCGGTGCT | | A7U | 6 |
| cr860 | GTTTAAGAGCTAAGCTGGAAACAGCATAGCAAGCTTAAATAAAGCTAGT | 1066 | U33C, | 0.01022314 |
| | CTGTTATCAACTTGAAAAAGTGGCACCGAGTCGGTGCT | | G42A, | 3 |
| | | | C50U | |
| cr949 | GTTTAAGAGCTAAGCTGGAAACAGCATAGCAAGGTTAAATAAGGCTAGT | 1067 | U33G, | 0.01022133 |
| | CCGTTATCAACTTGATAAAGTGGCACCGAGTCGGTGCT | | A64U | 3 |
| cr250 | GTTTAAGAGCTAAGCTGGAAACAGCATAGCAAGTTTAAATAAGGCTAGA | 1068 | U48A, | 0.01011888 |
| | CCGTTATCAACTTGAAAAAGTCGCACCGAGGCGGTGCT | | G70C, | |
| | | | U79G | |
| cr067 | CTTGAAGAGCTAAGCTGGAAACAGCATAGCAAGTTTAAATAAGGCTAGT | 1069 | G0C, | 0.01011543 |
| | CCGTTATCAACTTGAAAAAGTGGCACCGAGTCGGTGCT | | U3G | 6 |
| cr795 | GTTTAAGAGCTAAGCTGGAAACAGCATAGCAAGTTTAAATAATGCTAGT | 1070 | G42U, | 0.00991111 |
| | CAGTTATCAACTTGAAAAAGTCTCTCCGAGTCGGAGAT | | C50A, | 2 |
| | | | G71U, | |
| | | | A73U, | |
| | | | U83A, | |
| | | | C85A | |
| cr587 | GTTTAAGAGCTAAGCTGGAAACAGCATAGCAAGTTTAAATAAGCCTAGT | 1071 | G43C | 0.00980486 |
| | CCGTTATCAACTTGAAAAAGTGGCACCGAGTCGGTGCT | | | 6 |
| cr993 | GTTTAAGAGCTAAGCTGGAAACAGCATAGCAACTTTAAATAAGGCTAGT | 1072 | G32C, | 0.00968956 |
| | CCGTTATCATCTTGAAAAAGAGGCACCGAGTCGGTGCT | | A58U, | 5 |
| | | | U69A | |
| cr130 | GTTTAAGAGCTAAGCTGGAAACAGCATAGCATTTTTAAATAAGGCTAGT | 1073 | A31U, | 0.00950709 |
| | CCGTTATCAACTTGAAAAAGTGGCACCGAGTCGGTGCT | | G32U | 1 |
| cr328 | GTTTAAGAGCTAAGCTGGAAACAGCATAGCAACTTTAAATAAGGCTAGT | 1074 | G32C, | 0.00938366 |
| | CCGTTATCAACTTGAAAAAGTGGCCCCGAGTCGGGGCT | | A73C, | |
| | | | U83G | |
| cr187 | GTTTAAGAGCTAAGCTGGAAACAGCATAGCAAGGTTAAATAAGGCTAGT | 1075 | U33G, | 0.00928 |
| | CCGTTATCAACTTGAAAAAGTGGCACCAAGTTGGTGCT | | G76A, | |
| | | | C80U | |
| cr052 | ATGTAAGAGCTAAGCTGGAAACAGCATAGCAAGTTTAAATAAGGCTAGT | 1076 | G0A, | 0.00923802 |
| | CCGTTATCAACTTGAAAAAGTGGCACCGAGTCGGTGCT | | U2G | 7 |
| cr081 | ATTAAAGAGCTAAGCTGGAAACAGCATAGCAAGTTTAAATAAGGCTAGT | 1077 | G0A, | 0.00920730 |
| | CCGTTATCAACTTGAAAAAGTGGCACCGAGTCGGTGCT | | U3A | 9 |
| cr114 | GTTTAAGAGCTAAGCTGGAAACAGCATAGCAAGTTTAAATAATGTTAATC | 1078 | G42U, | 0.00858876 |
| | AGTTATCAACTTGAAAAAGTGGCCCCGAGTCGGGGCT | | C44U, | 5 |
| | | | G47A, | |
| | | | C50A, | |
| | | | A73C, | |
| | | | U83G | |
| cr137 | GTTTAAGAGCTAAGCTGGAAACAGCATAGCAAGTTTAAATAGGGCTAGT | 1079 | A41G, | 0.00851592 |
| | CCTTTATCAACTTGCAAAAGTGGCACCGAGTCGGTGCT | | G51U, | 2 |
| | | | A63C | |
| cr648 | GTTTAAGAGCTAAGCTGGAAACAGCATAGCAACTTTAAATAAGTCTAGT | 1080 | G32C, | 0.00830519 |
| | ACGTDVTCAACTTGAAAAAGTGGCACCGAGTCGGTGCT | | G43U, | 3 |
| | | | C49A | |
| cr295 | GTTTAAGAGCTAAGCTGGAAACAGCATAGCAAGGTTAAATAAGGCTAGT | 1081 | U33G, | 0.00826811 |
| | CCGTTATCAACTTGAAAAAGTGGGACCGAGTCGGTCCT | | C72G, | 1 |
| | | | G84C | |
| cr436 | GTTTAAGAGCTAAGCTGGAAACAGCATAGCAAGGTTAAATAAGGCTAGT | 1082 | U33G, | 0.00812877 |
| | CCGTTATCATCTTGAAAAAGAGGCACCGAGTCGGTGCT | | A58U, | 9 |
| | | | U69A | |
| cr862 | GTTTAAGAGCTAAGCTGGAAACAGCATATCACCTTTAAATAAGGCTAGTC | 1083 | G28U, | 0.00788739 |
| | CGTTATCAACTTGAAAAAGTGGCACCGAGTCGGTGCT | | A31C, | 2 |
| | | | G32C | |
| cr160 | GTTTAAGAGCTAAGCTGGAAACAGCATAGCAATCTTAAATAAGGCTAGT | 1084 | G32U, | 0.00787920 |
| | CCGTTATCAACTTGAAAAAGTGGCACCGAGTCGGTGCT | | U33C | 4 |
| cr807 | GTTTAAGAGCTAAGCTGGAAACAGCATAGCAAGTTTAAATTAGGCTAGT | 1085 | A40U, | 0.00776015 |
| | CCGTTATCAACTTGAAAAAGTGGCACCGTGTCGGTGCT | | A77U | 7 |
| cr664 | GTTTAAGAGCTAAGCTGGAAACAGCATAGCAACCTTAAATAAGGCTAGT | 1086 | G32C, | 0.00743456 |
| | CCGTTATCAACTTGAAAAAGTGGCACCGAGTCGGTGCT | | U33C | 4 |
| cr512 | GTTTAAGAGCTAAGCTGGAAACAGCATAGCAAGGTTAAATAAGCCTAGT | 1087 | U33G, | 0.00741391 |
| | GCGTTATCAACTTGAAAAAGTGGCACCGAGTCGGTGCT | | G43C, | 6 |
| | | | C49G | |
| cr415 | GTTTAAGAGCTAAGCTGGAAACAGCATAGCAAGTTTAAATAAGGCTAGT | 1088 | G51C, | 0.00728079 |
| | CCCTTATCAACTTGAAATAGTGGCACCGAGTCGGTGCT | | A66U | 1 |
| cr499 | GTTTAAGAGCTAAGCTGGAAACAGCATAGCAACTTTAAATAAGGCTAGT | 1089 | G32C, | 0.00725709 |
| | CCGATATCAACTTGAAAAAGTGGCACCGAGGCGGTGCT | | U52A, | 5 |
| | | | U79G | |
| cr778 | GTTTAAGAGCTAAGCTGGAAACAGCATAGCAAGTTTAAATAAGGCTAGT | 1090 | C50G | 0.00719960 |
| | CGGTTATCAACTTGAAAAAGTGGCACCGAGTCGGTGCT | | | 6 |
| cr339 | GTTTAAGAGCTAAGCTGGAAACAGCATAGCAAGTTTAAATAACCCTAGT | 1091 | G42C, | 0.00717030 |
| | GGGTTATCAACTTGAAAAAGTGGCACCGAGTCGGTGCT | | G43C, | 9 |
| | | | C49G, | |
| | | | C50G | |
| cr247 | GTTTAAGAGCTAAGCTGGAAACAGCATAGCAATTTTAAATAAGGCTAGT | 1092 | G32U, | 0.00715877 |
| | CCGTTATCAACTTGAAAAAGTAGCACCGAGTCGGTGCT | | G70A | 9 |
| cr883 | GTTTAAGAGCTAAGCTGGAAACAGCATAGCAAGTTTAAATAGGGCTAGT | 1093 | A41G, | 0.00697489 |
| | CGGTTATCAACTTGAAAAAGTGGCACCGAGTCGGTGCT | | C50G | 1 |
| cr376 | GTTTAAGAGCTAAGCTGGAAACAGCATAGCAACTTTAAATAAGGCTAGT | 1094 | G32C, | 0.00696306 |
| | CCGTTATCAACTTGAAAAAGTGGCACGGAGTCCGTGCT | | C75G, | 3 |
| | | | G81C | |
| cr518 | GTTTAAGAGCTAAGCTGGAAACAGCATAGCAATTTTAAATGAGGCTAGT | 1095 | G32U, | 0.00688861 |
| | CCGTTATCAACTTGAAAAAGTGGCACCGAGTCGGTGCT | | A40G | |
| cr092 | GTGTAGGAGCTAAGCTGGAAACAGCATAGCAAGTTTAAATAAGGCTAGT | 1096 | U2G, | 0.00687503 |
| | CCGTTATCAACTTGAAAAAGTGGCACCGAGTCGGTGCT | | A5G | 1 |
| cr281 | GTTTAAGAGCTAAGCTGGAAACAGCATAGCAAGTTTAAATAAGGCTAGT | 1097 | A73U, | 0.00680234 |
| | CCGTTATCAACTTGAAAAAGTGGCTGGCAGTCCGAGCT | | C74G, | 1 |
| | | | C75G, | |
| | | | G76C, | |
| | | | G81C, | |
| | | | U83A | |
| cr463 | GTTTAAGAGCTAAGCTGGAAACAGCATAGCAAGTTTAAATAACGCTAGT | 1098 | G42C, | 0.00679918 |
| | CGGTTATCACCTTGAAAAAGGGGCACCTAGTAGGTGCT | | C50G, | 1 |
| | | | A58C, | |
| | | | U69G, | |
| | | | G76U, | |
| | | | C80A | |
| cr174 | GTTTAAGAGCTAAGCTGGAAACAGCATACCAAGTTTAAATAAGGCTAGG | 1099 | G28C, | 0.00666861 |
| | CCGTTATCAACTTGAAAAAGTGGCACCGATTCGGTGCT | | U48G, | 1 |
| | | | G78U | |
| cr706 | GTTTAAGAGCTAAGCTGGAAACAGCATAGCAAGGTTAAATAAGGCTAGT | 1100 | U33G, | 0.00659509 |
| | CCGTTATCAACTTGAAAAAGTGGCACGGAGTCCGTGCT | | C75G, | |
| | | | G81C | |
| cr967 | GTTTAAGAGCTAAGCTGGAAACAGCATAGCAAGTTTAAATAACGCTAGT | 1101 | G42C, | 0.00644569 |
| | CGGTTATCAACTTGAAAAAGTGGAACCAAGTTGGTTCT | | C50G, | 5 |
| | | | C72A, | |
| | | | G76A, | |
| | | | C80U, | |
| | | | G84U | |
| cr272 | GTTTAAGAGCTAAGCTGGAAACAGCATAGCCACTTTAAATAAGGCTAGT | 1102 | A30C, | 0.00637966 |
| | CCGTTATCAACTTGAAAAAGTGGCACCGAGTCGGTGCT | | G32C | 2 |
| cr744 | GTTTAAGAGCTAAGCTGGAAACAGCATAGCAAGATTAAATCAGGCTAGT | 1103 | U33A, | 0.00634918 |
| | CCGTTATCAACTTGTAAAAGTGGCACCGAGTCGGTGCT | | A40C, | 4 |
| | | | A63U | |
| cr615 | GTTTAAGAGCTAAGCTGGAAACAGCATAGCAAGTTTAAATAAGGCTAGT | 1104 | G71C, | 0.00633273 |
| | CCGTTATCAACTTGAAAAAGTGCGTGCGAGTCGGAGGT | | C72G, | 6 |
| | | | A73U, | |
| | | | C74G, | |
| | | | U83A, | |
| | | | C85G | |
| cr100 | GTTTAAGAGCTAAGCTGGAAACAGCATAGCAAGTTTAAATGAGGCTAGC | 1105 | A40G, | 0.00592343 |
| | CCGTTATCAACTTGAAAAAGTGGCACCGAGTCGGTGCT | | U48C | 9 |
| cr584 | GTTTAAGAGCTAAGCTGGAAACAGCATAGCAAGTTTAAATATGGCTAGT | 1106 | A41U, | 0.00591156 |
| | CCTTTATCAACTTGAAAAAGTGGCACCGAGTCGGTGCT | | G51U | 2 |
| cr057 | GCTCAAGAGCTAAGCTGGAAACAGCATAGCAAGTTTAAATAAGGCTAGT | 1107 | U1C, | 0.00575747 |
| | CCGTTATCAACTTGAAAAAGTGGCACCGAGTCGGTGCT | | U3C | |
| cr266 | GTTTAAGAGCTAAGCTGGAAACAGCATGGCAAGGTTAAATAAGGCGAG | 1108 | A27G, | 0.00555163 |
| | TCCGTTATCAACTTGAAAAAGTGGCACCGAGTCGGTGCT | | U33G, | 7 |
| | | | U45G | |
| cr537 | GTTTAAGAGCTAAGCTGGAAACAGCATAGCAAGCTTAAATTAGGCTAGT | 1109 | U33C, | 0.00527642 |
| | CCGTTATCAACTTGAAAAAGTGGCACCGAGTCGGTGCT | | A40U | 2 |
| cr060 | GTGCAAGAGCTAAGCTGGAAACAGCATAGCAAGTTTAAATAAGGCTAGT | 1110 | U2G, | 0.00518162 |
| | CCGTTATCAACTTGAAAAAGTGGCACCGAGTCGGTGCT | | U3C | 2 |
| cr309 | GTTTAAGAGCTAAGCTGGAAACAGCATAGCAAGGTTAAATAAGGCTAGG | 1111 | U33G, | 0.00504472 |
| | CCGTTATCAACTTGAAAAAGTGGCACCGAGTCGGTGCT | | U48G | 8 |
| cr472 | GTTTAAGAGCTAAGCTGGAAACAGCATAGCAAGGTTAAATAAGGTTAAT | 1112 | U33G, | 0.00491748 |
| | CCGTTATCAACTTGAAAAAGTGGCACCGAGTCGGTGCT | | C44U, | 3 |
| | | | G47A | |
| cr297 | GTTTAAGAGCTAAGCTGGAAACAGCATAGCAAGTTTAAATAAGGCTAGG | 1113 | U48G, | 0.00487154 |
| | CCGTTATCAACTTGAAAAAGTCGCACCGAGTCGGTGCT | | G70C | 3 |
| cr849 | GTTTAAGAGCTAAGCTGGAAACAGCATAGCAACGTTAAATAAGGCTAGT | 1114 | G32C, | 0.00470573 |
| | CCGTTATCAACTTGAAAAAGTGGCACCGAGTCGGTGCT | | U33G | 8 |
| cr845 | GTTTAAGAGCTAAGCTGGAAACAGCATAGCAAGGTTAAATAAAGCTAGT | 1115 | U33G, | 0.00464434 |
| | CTGTTATCAACTTGAAAAAGTGGCACCGAGTCGGTGCT | | G42A, | 1 |
| | | | C50U | |
| cr400 | GTTTAAGAGCTAAGCTGGAAACAGCATAGCAAGGTTAAATAAGGATATT | 1116 | U33G, | 0.00451030 |
| | CCGTTATCAACTTGAAAAAGTGGCACCGAGTCGGTGCT | | C44A, | 5 |
| | | | G47U | |
| cr094 | GATTTAGAGCTAAGCTGGAAACAGCATAGCAAGTTTAAATAAGGCTAGT | 1117 | U1A, | 0.00441375 |
| | CCGTTATCAACTTGAAAAAGTGGCACCGAGTCGGTGCT | | A4U | 1 |
| cr203 | GTTTAAGAGCTAAGCTGGAAACAGCATAGCAACTTTAAATAAGGCTAGT | 1118 | G32C, | 0.00433309 |
| | CCTTTATCAACTTGAAAAAGTGGCACCGAGTCGGTGCT | | G51U | |
| cr753 | GTTTAAGAGCTAAGCTGGAAACAGCATAGCAAGTTTAAATAACGCTAGT | 1119 | G42C, | 0.00430245 |
| | CGGTTATCATCTTGAAAAAGAGGAACCGAGTCGGTTCT | | C50G, | |
| | | | A58U, | |
| | | | U69A, | |
| | | | C72A, | |
| | | | G84U | |
| cr857 | GTTTAAGAGCTAAGCTGGAAACAGCATAGCAAGTTTAAATTAGGCTAGT | 1120 | A40U, | 0.00427408 |
| | CCGTTATCAACTTGAAAAAGTCGCACCGAGCCGGTGCT | | G70C, | |
| | | | U79C | |
| cr833 | GTTTAAGAGCTAAGCTGGAAACAGCATCGCAACTTTAAATAAGGCTAGT | 1121 | A27C, | 0.00421504 |
| | CCGTTATCAACTTGAAAAAGTCGCACCGAGTCGGTGCT | | G32C, | 6 |
| | | | G70C | |
| cr660 | GTTTAAGAGCTAAGCTGGAAACAGCATAGCAAGTTTAAATAATGCTAGT | 1122 | G42U | 0.00410246 |
| | CCGTTATCAACTTGAAAAAGTGGCACCGAGTCGGTGCT | | | 1 |
| cr536 | GTTTAAGAGCTAAGCTGGAAACAGCATAGCAACTTTAAATAAGGATATT | 1123 | G32C, | 0.00395083 |
| | CCGTTATCAACTTGAAAAAGTGGCACCGAGTCGGTGCT | | C44A, | 1 |
| | | | G47U | |
| cr245 | GTTTAAGAGCTAAGCTGGAAACAGCATAGCAAGGTTAAATAAGACTAGT | 1124 | U33G, | 0.00372006 |
| | TCGTTATCAACTTGAAAAAGTGGCACCGAGTCGGTGCT | | G43A, | 4 |
| | | | C49U | |
| cr303 | GTTTAAGAGCTAAGCTGGAAACAGCATAGCAACTTTAAATAAGGCTTGT | 1125 | G32C, | 0.00357317 |
| | CCGTTATCAACTTGAAAAAGTTGCACCGAGTCGGTGCT | | A46U, | 9 |
| | | | G70U | |
| cr981 | GTTTAAGAGCTAAGCTGGAAACAGCATAGCAAGTTTAAATAACGCTAGT | 1126 | G42C, | 0.00326636 |
| | CGGTTATCAACTGGAAACAGTGGCACCGAGTCGGTGCT | | C50G, | 7 |
| | | | U61G, | |
| | | | A66C | |
| cr469 | GTTTAAGAGCTAAGCTGGAAACAGCATAGCAAGTTTAAATAAGGCTAGT | 1127 | G51C | 0.00321140 |
| | CCCTTATCAACTTGAAAAAGTGGCACCGAGTCGGTGCT | | | 6 |
| cr049 | GTGTTAGAGCTAAGCTGGAAACAGCATAGCAAGTTTAAATAAGGCTAGT | 1128 | U2G, | 0.00320308 |
| | CCGTTATCAACTTGAAAAAGTGGCACCGAGTCGGTGCT | | A4U | 7 |
| cr381 | GTTTAAGAGCTAAGCTGGAAACAGCATAGCAAGTTTAAATTAGGCTCGT | 1129 | A40U, | 0.00319558 |
| | CCGTTATCAACTTGACAAAGTGGCACCGAGTCGGTGCT | | A46C, | 2 |
| | | | A64C | |
| cr482 | GTTTAAGAGCTAAGCTGGAAACAGCATAGCAAGTTTAAATAAGGCTTGT | 1130 | A46U, | 0.00300577 |
| | CCCTTATCAACTTGAAAAAGTGGCACCGAGTCGGTGCT | | G51C | 4 |
| cr068 | GTTAGAGAGCTAAGCTGGAAACAGCATAGCAAGTTTAAATAAGGCTAGT | 1131 | U3A, | 0.00295873 |
| | CCGTTATCAACTTGAAAAAGTGGCACCGAGTCGGTGCT | | A4G | 7 |
| cr674 | GTTTAAGAGCTAAGCTGGAAACAGCATAGCAAGTTTAAATAACGCTAGT | 1132 | G42C, | 0.00279049 |
| | CGGTTATCATCTTGAAAAAGAGGCACCGAGTCGGTGCT | | C50G, | 7 |
| | | | A58U, | |
| | | | U69A | |
| cr572 | GTTTAAGAGCTAAGCTGGAAACAGCATAGCAAGTTTAAATAACCGAACT | 1133 | G42C, | 0.00278236 |
| | CGGTTATCAACTTGAAAAAGTGGCACCGAGTCGGTGCT | | G43C, | 4 |
| | | | C44G, | |
| | | | U45A, | |
| | | | G47C, | |
| | | | C50G | |
| cr225 | GTTTAAGAGCTAAGCTGGAAACAGCATAGCAAGTTTAAATAATGCTATTC | 1134 | G42U, | 0.00265752 |
| | CGTTATCAACTTGAAAAAGTGGCACCGAGTCGGTGCT | | G47U | 7 |
| cr657 | GTTTAAGAGCTAAGCTGGAAACAGCATAGCAAGTTTAAATAAGGCTAGT | 1135 | C49G | 0.00260388 |
| | GCGTTATCAACTTGAAAAAGTGGCACCGAGTCGGTGCT | | | 8 |
| cr735 | GTTTAAGAGCTAAGCTGGAAACAGCATAGCAACTTTAAATAAGGCTAGT | 1136 | G32C, | 0.00259569 |
| | CCGTTATCAACTTGAAAAAGTCGCACCGAGTCGGTGCT | | G70C | 5 |
| cr608 | GTTTAAGAGCTAAGCTGGAAACAGCATAGCAACTTTAAATAAGGGTACT | 1137 | G32C, | 0.00256753 |
| | CCGTTATCAACTTGAAAAAGTGGCACCGAGTCGGTGCT | | C44G, | 3 |
| | | | G47C | |
| cr903 | GTTTAAGAGCTAAGCTGGAAACAGCATAGCAAGTTTAAATAAGGCTAGT | 1138 | G51C, | 0.00221972 |
| | CCCTTATCAACTTGAAAAAGTGGCACCGAATCGGTGCT | | G78A | 6 |
| cr975 | GTTTAAGAGCTAAGCTGGAAACAGCATAGCAACTTTAAATATGGCTAGT | 1139 | G32C, | 0.00215224 |
| | CCGTTATCAACTTGAAAAAGTGGCACCGAGTCGGTGCT | | A41U | 7 |
| cr874 | GTTTAAGAGCTAAGCTGGAAACAGCATAGCAAGGTTAAATAATGCTAGT | 1140 | U33G, | 0.00214364 |
| | CAGTTATCAACTTGAAAAAGTGGCACCGAGTCGGTGCT | | G42U, | 1 |
| | | | C50A | |
| cr810 | GTTTAAGAGCTAAGCTGGAAACAGCATAGCAAGGTTAAATAAGGCTAGT | 1141 | U33G, | 0.00202570 |
| | CCGTTATCAACTTGAAAAAGTGGCACCCAGTGGGTGCT | | G76C, | 2 |
| | | | C80G | |
| cr355 | GTTTAAGAGCTAAGCTGGAAACAGCATAGCAAGGTTAAATAAGGCTAGT | 1142 | U33G, | 0.00177064 |
| | CCGTTATCAACTAGAAATAGTGGCACCGAGTCGGTGCT | | U61A, | 4 |
| | | | A66U | |
| cr786 | GTTTAAGAGCTAAGCTGGAAACAGCATAGCAAGTTTAAATTAGGCTAGT | 1143 | A40U, | 0.00174934 |
| | CCGTTGTGAACTTGAAAAAGTGGCACCGAGTCGGTGCT | | A54G, | 8 |
| | | | C56G | |
| cr930 | GTTTAAGAGCTAAGCTGGAAACAGCATAGCATGTTTAAATAAGGCTAGG | 1144 | A31U, | 0.00172050 |
| | CCGTTATTAACTTGAAAAAGTGGCACCGAGTCGGTGCT | | U48G, | 2 |
| | | | C56U | |
| cr149 | GTTTAAGAGCTAAGCTGGAAACAGCATAGCAAGTTTAAATAACGCTAGT | 1145 | G42C, | 0.00166965 |
| | CGGTTATCAACTTGAAAAAGTGTCACCGAGTCGGTGAT | | C50G, | 2 |
| | | | G71U, | |
| | | | C85A | |
| cr143 | GTTTAAGAGCTAAGCTGGAAACAGCATAGCAACTTTAAATAAGACTAGT | 1146 | G32C, | 0.00152365 |
| | TCGTTATCAACTTGAAAAAGTGGCACCGAGTCGGTGCT | | G43A, | 1 |
| | | | C49U | |
| cr244 | GTTTAAGAGCTAAGCTGGAAACAGCATAGCAAGGTTAAATAAGGGTACT | 1147 | U33G, | 0.00149049 |
| | CCGTTATCAACTTGAAAAAGTGGCACCGAGTCGGTGCT | | C44G, | 5 |
| | | | G47C | |
| cr093 | GTTTCGGAGCTAAGCTGGAAACAGCATAGCAAGTTTAAATAAGGCTAGT | 1148 | A4C, | 0.00145631 |
| | CCGTTATCAACTTGAAAAAGTGGCACCGAGTCGGTGCT | | A5G | 9 |
| cr350 | GTTTAAGAGCTAAGCTGGAAACAGCATAGCAAGCTTAAATAATGCTAGT | 1149 | U33C, | 0.00142283 |
| | CAGTTATCAACTTGAAAAAGTGGCACCGAGTCGGTGCT | | G42U, | |
| | | | C50A | |
| cr083 | GTGTGAGAGCTAAGCTGGAAACAGCATAGCAAGTTTAAATAAGGCTAGT | 1150 | U2G, | 0.00140262 |
| | CCGTTATCAACTTGAAAAAGTGGCACCGAGTCGGTGCT | | A4G | |
| cr106 | GTTTAAGAGCTAAGCTGGAAACAGCATAGCAAGGTTAAATAAGTCTAGT | 1151 | U33G, | 0.00135486 |
| | ACGTTATCAACTTGAAAAAGTGGCACCGAGTCGGTGCT | | G43U, | 3 |
| | | | C49A | |
| cr787 | GTTTAAGAGCTAAGCTGGAAACAGCATAGCAACTTTAAATAACGCTAGT | 1152 | G32C, | 0.00084829 |
| | CGGTTATCAACTTGAAAAAGTGGCACCGAGTCGGTGCT | | G42C, | 5 |
| | | | C50G | |
| cr035 | GTTGATGAGCTAAGCTGGAAACAGCATAGCAAGTTTAAATAAGGCTAGT | 1153 | U3G, | 0.00069273 |
| | CCGTTATCAACTTGAAAAAGTGGCACCGAGTCGGTGCT | | A5U | 1 |
| cr099 | GTTTAAGAGCTAAGCTGGAAACAGCATAGCAATTTTAAATAAGGCTAGG | 1154 | G32U, | 0.00050524 |
| | CCGTTATCAACTTGAAAAAGTGGCACCGAGTCGGTGCT | | U48G | 3 |
| cr939 | GTTTAAGAGCTAAGCTGGAAACAGCATAGCAACTTTAAATAAGGCAAGT | 1155 | G32C, | 4.94E−05 |
| | CCCTTATCAACTTGAAAAAGTGGCACCGAGTCGGTGCT | | U45A, | |
| | | | G51C | |
| cr912 | GTTTAAGAGCTAAGCTGGAAACAGCATAGCAAGCTTAAATGAGGCTAGT | 1156 | U33C, | |
| | CCGGTATCAACTTGAAAAAGTGGCACCGAGTCGGTGCT | | A40G, | −1.05E−05 |
| | | | U52G | |
| cr629 | GTTTAAGAGCTAAGCTGGAAACAGCATAGCAACTTTAAATAAGGCTAGT | 1157 | G32C, | |
| | CCGTTATCAAATTGAAAAATTGGCACCGAGTCGGTGCT | | C59A, | −5.97E−5 |
| | | | G68U | |
| cr431 | GTTTAAGAGCTAAGCTGGAAACAGCATAGCAAGGTTAAATAAGGCTAGT | 1158 | U33G, | −0.00027128 |
| | CCGTTATCAACTTGAGAAAGTGGCACCGCGTCGGTGCT | | A64G, | |
| | | | A77C | |
| cr579 | GTTTAAGAGCTAAGCTGGAAACAGCATAGCAAGTTTAAATAAGGCTAGT | 1159 | G51C, | −0.00041798 |
| | CCCATATCAACTTGAAAAAGTGGCACCGAGTCGGTGCT | | U52A | 5 |
| cr535 | GTTTAAGAGCTAAGCTGGAAACAGCATAGCAAGTTTAAATAAGGCTAGT | 1160 | C50A | −0.00042808 |
| | CAGTTATCAACTTGAAAAAGTGGCACCGAGTCGGTGCT | | | 4 |
| cr751 | GTTTAAGAGCTAAGCTGGAAACAGCATCGCAACTTTAAATAAGGCTAGT | 1161 | A27C, | −0.00055700 |
| | CCTTTATCAACTTGAAAAAGTGGCACCGAGTCGGTGCT | | G32C, | 4 |
| | | | G51U | |
| cr039 | GATTATGAGCTAAGCTGGAAACAGCATAGCAAGTTTAAATAAGGCTAGT | 1162 | U1A, | −0.00061806 |
| | CCGTTATCAACTTGAAAAAGTGGCACCGAGTCGGTGCT | | A5U | 4 |
| cr178 | GTTTAAGAGCTAAGCTGGAAACAGCATAGCAATTTTAAATGAGGCTAGT | 1163 | G32U, | −0.00087438 |
| | CCGTTATCGACTTGAAAAAGTGGCACCGAGTCGGTGCT | | A40G, | 9 |
| | | | A57G | |
| cr326 | GTTTAAGAGCTAAGCTGGAAACAGCATAGCAAGCTTAAATAACGCTAGT | 1164 | U33C, | −0.00106221 |
| | CGGTTATCAACTTGAAAAAGTGGCACCGAGTCGGTGCT | | G42C, | 6 |
| | | | C50G | |
| cr637 | GTTTAAGAGCTAAGCTGGAAACAGCATATCAAGATTAAATAAGGCTAGT | 1165 | G28U, | −0.00126524 |
| | CCGTTATCAACTTGAGAAAGTGGCACCGAGTCGGTGCT | | U33A, | |
| | | | A64G | |
| cr564 | GTTTAAGAGCTAAGCTGGAAACAGCATAGCAAGGTTAAATAAGGCTAGT | 1166 | U33G, | −0.00195195 |
| | CCGTTATCAACTTGAAAAAGTGGCAGCGAGTCGCTGCT | | C74G, | 2 |
| | | | G82C | |
| cr921 | GTTTAAGAGCTAAGCTGGAAACAGCATAGCAAAGTTAAATAAGGCTAGT | 1167 | G32A, | −0.00220269 |
| | CCGTTATCAACTTGAAAAAGTGGCACCGAGTCGGTGCT | | U33G | 5 |
| cr817 | GTTTAAGAGCTAAGCTGGAAACAGCATAGCAAGGTTAAATAGGGCTAGT | 1168 | U33G, | −0.00251447 |
| | CCGTTATCAACTTGATAAAGTGGCACCGAGTCGGTGCT | | A41G, | 3 |
| | | | A64U | |
| cr847 | GTTTAAGAGCTAAGCTGGAAACAGCATAGCAAGCTTAAATAAGGCTAGT | 1169 | U33C, | −0.00268195 |
| | CCGTTATCAACTTGAAAAAGTGGCACCCAGTGGGTGCT | | G76C, | 9 |
| | | | C80G | |
| cr595 | GTTTAAGAGCTAAGCTGGAAACAGCATAGCAAGTTTAAATAACGCTAGT | 1170 | G42C | −0.00298431 |
| | CCGTTATCAACTTGAAAAAGTGGCACCGAGTCGGTGCT | | | 1 |
| cr065 | GTATACGAGCTAAGCTGGAAACAGCATAGCAAGTTTAAATAAGGCTAGT | 1171 | U2A, | −0.00314793 |
| | CCGTTATCAACTTGAAAAAGTGGCACCGAGTCGGTGCT | | A5C | 3 |
| cr058 | GTTGCAGAGCTAAGCTGGAAACAGCATAGCAAGTTTAAATAAGGCTAGT | 1172 | U3G, | −0.00315143 |
| | CCGTTATCAACTTGAAAAAGTGGCACCGAGTCGGTGCT | | A4C | |
| cr894 | GTTTAAGAGCTAAGCTGGAAACAGCATAGCAACTTTAAATAAGGCTAGT | 1173 | G32C, | −0.00340798 |
| | CCGTTATCAATTTGAAAAAATGGCACCGAGTCGGTGCT | | C59U, | 4 |
| | | | G68A | |
| cr122 | GTTTAAGAGCTAAGCTGGAAACAGCATAGCAACTTTAAATAAGGCTAGT | 1174 | G32C, | −0.00340841 |
| | CCGTTATTAACTTGATAAAGTGGCACCGAGTCGGTGCT | | C56U, | 6 |
| | | | A64U | |
| cr233 | GTTTAAGAGCTAAGCTGGAAACAGCATAGCAACTTTAAATAAGGCTAGT | 1175 | G32C, | −0.00351086 |
| | CCGTTATCAACTTGAAAAAGTGGCACCCAGTGGGTGCT | | G76C, | 1 |
| | | | C80G | |
| cr053 | GATTCAGAGCTAAGCTGGAAACAGCATAGCAAGTTTAAATAAGGCTAGT | 1176 | U1A, | −0.00370399 |
| | CCGTTATCAACTTGAAAAAGTGGCACCGAGTCGGTGCT | | A4C | 7 |
| cr686 | GTTTAAGAGCTAAGCTGGAAACAGCATAGCAACTTTAAATAAGGCTAGT | 1177 | G32C, | −0.00401893 |
| | CCGTTATCAACTTGAAAAAGTGGCACCGAGACGGTGCT | | U79A | 5 |
| cr179 | GTTTAAGAGCTAAGCTGGAAACAGCATAGCAAGGTTAAATAAGGCTAGT | 1178 | U33G, | −0.00449278 |
| | CCGTTATCAACTTGAAAAAGTGGCACTGAGTCAGTGCT | | C75U, | 7 |
| | | | G81A | |
| cr033 | GTTAAAAAGCTAAGCTGGAAACAGCATAGCAAGTTTAAATAAGGCTAGT | 1179 | U3A, | −0.00465615 |
| | CCGTTATCAACTTGAAAAAGTGGCACCGAGTCGGTGCT | | G6A | 9 |
| cr673 | GTTTAAGAGCTAAGCTGGAAACAGCATAGCAACTTTAAATAAGGCTAGT | 1180 | G32C, | −0.00507235 |
| | CCGTTATCAACTTGAAAAAGTGGCACCGTGTCGGTGCT | | A77U | 7 |
| cr589 | GTTTAAGAGCTAAGCTGGAAACAGCATAGCAAGTTTAAATAATGATATTC | 1181 | G42U, | −0.00510801 |
| | AGTTATCAACTTGAAAAAGTGGCACCGAGTCGGTGCT | | C44A, | |
| | | | G47U, | |
| | | | C50A | |
| cr802 | GTTTAAGAGCTAAGCTGGAAACAGCATAGCAAGTTTAAATAACGCTAGT | 1182 | G42C, | −0.00537686 |
| | CGGTTATCAAGTTGAAAAACTGGCAGCGAGTCGCTGCT | | C50G, | 5 |
| | | | C59G, | |
| | | | G68C, | |
| | | | C74G, | |
| | | | G82C | |
| cr383 | GTTTAAGAGCTAAGCTGGAAACAGCATAGCAACTTTAAATAAGGCTAGT | 1183 | G32C, | −0.00550463 |
| | CCGTTATCAACTTGAAAAAGTGGCACCTAGTAGGTGCT | | G76U, | 1 |
| | | | C80A | |
| cr036 | GCTAAAGAGCTAAGCTGGAAACAGCATAGCAAGTTTAAATAAGGCTAGT | 1184 | U1C, | −0.00567874 |
| | CCGTTATCAACTTGAAAAAGTGGCACCGAGTCGGTGCT | | U3A | |
| cr034 | GCATAAGAGCTAAGCTGGAAACAGCATAGCAAGTTTAAATAAGGCTAGT | 1185 | U1C, | −0.00719105 |
| | CCGTTATCAACTTGAAAAAGTGGCACCGAGTCGGTGCT | | U2A | 1 |
| cr329 | GTTTAAGAGCTAAGCTGGAAACAGCATAGCAACTTTAAATAAAGCTAGT | 1186 | G32C, | −0.00760657 |
| | CTGTTATCAACTTGAAAAAGTGGCACCGAGTCGGTGCT | | G42A, | 1 |
| | | | C50U | |
| cr135 | GTTTAAGAGCTAAGCTGGAAACAGCATAGCAAGTTTAAATAACGCTAGT | 1187 | G42C, | −0.00878040 |
| | CGGTTATCAAATTGAAAAATTGGCGCCGAGTCGGCGCT | | C50G, | 6 |
| | | | C59A, | |
| | | | G68U, | |
| | | | A73G, | |
| | | | U83C | |
| cr056 | GTGTCAGAGCTAAGCTGGAAACAGCATAGCAAGTTTAAATAAGGCTAGT | 1188 | U2G, | −0.00951153 |
| | CCGTTATCAACTTGAAAAAGTGGCACCGAGTCGGTGCT | | A4C | 1 |
| cr583 | GTTTAAGAGCTAAGCTGGAAACAGCATAGCAAGTTTAAATCAGGCTAGT | 1189 | A40C, | −0.01012720 |
| | CCATTATCTACTTGAAAAAGTGGCACCGAGTCGGTGCT | | G51A, | 7 |
| | | | A57U | |
| cr661 | GTTTAAGAGCTAAGCTGGAAACAGCATAACAAGTTTAAATAAGGCTAGT | 1190 | G28A, | −0.01151227 |
| | CCCTTATCAACTTGAATAAGTGGCACCGAGTCGGTGCT | | G51C, | 7 |
| | | | A65U | |
| cr784 | GTTTAAGAGCTAAGCTGGAAACAGCATAGCAAGTTTAAATAATCCTAGTT | 1191 | G42U, | −0.01212699 |
| | CGTTATCAACTTGAAAAAGTGGCACCGAGTCGGTGCT | | G43C, | 9 |
| | | | C49U | |
| cr540 | GTTTAAGAGCTAAGCTGGAAACAGCATAGCAACTTTAAATAATGCTAGTC | 1192 | G32C, | −0.01220390 |
| | AGTTATCAACTTGAAAAAGTGGCACCGAGTCGGTGCT | | G42U, | 3 |
| | | | C50A | |
| cr990 | GTTTAAGAGCTAAGCTGGAAACAGCATATCAAGCTTAAATAAGGCTAGT | 1193 | G28U, | −0.01629740 |
| | CCGTTATGAACTTGAAAAAGTGGCACCGAGTCGGTGCT | | U33C, | 7 |
| | | | C56G | |
| cr790 | GTTTAAGAGCTAAGCTGGAAACAGCATAGCAACTTTAAATAAGGCTAGT | 1194 | G32C, | −0.0169697 |
| | CCGTTATCAACTTGAAAAAGTGGCACCAAGTTGGTGCT | | G76A, | |
| | | | C80U | |
| cr551 | GTTTAAGAGCTAAGCTGGAAACAGCTTAGCAAGTTTAAATAAGGCTAGT | 1195 | A25U | −0.10897159 |
| | CCGTTATCAACTTGAAAAAGTGGCACCGAGTCGGTGCT | | | 1 |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of generating a set of single guide RNAs (sgRNAs) capable of driving a series of discrete expression levels of a target gene in a cell population using CRISPR interference (CRISPRi) or CRISPR activation (CRISPRa), the method comprising:
(i) providing a first sgRNA that targets the gene, wherein the last 19 nucleotides of the targeting sequence of the first sgRNA are 100% homologous to the target DNA sequence;
(ii) providing a second sgRNA that targets the gene, wherein the last 19 nucleotides of the targeting sequence of the second sgRNA comprises one or more mismatches with the target DNA sequence such that the CRISPRi or CRISPRa activity on the gene obtained using the second sgRNA is intermediate between that obtained using the first sgRNA and that obtained using a scrambled sgRNA providing no CRISPRi or CRISPRa activity on the gene; and
(iii) providing a third sgRNA that targets the gene, wherein the last 19 nucleotides of the targeting sequence of the third sgRNA comprises one or more mismatches with the target DNA sequence such that the CRISPRi or CRISPRa activity on the gene obtained using the third sgRNA is intermediate between that obtained using the second sgRNA and that obtained using a scrambled sgRNA providing no CRISPRi or CRISPRa activity on the gene;
wherein the mismatches of the second and third sgRNAs are selected according to the following rules:
(a) the CRISPRi or CRISPRa activity of the second sgRNA is designed to be greater than that of the third sgRNA based on the following positional relationships, wherein the positions correspond to the number of bases in the sgRNAs upstream from the sgRNA PAM:
−19>−18>−17>−16≈−15≈−14>−13>−12>−11>−10>−9>−8>−4>−7≈−6≈−5≈−3≈−2≈−1; or
(b) the CRISPRi or CRISPRa activity of the second sgRNA is designed to be greater than that of the third sgRNA based on the following base pair rankings of the mismatched nucleotides, wherein the first nucleotide in each pair corresponds to the ribonucleotide within the sgRNA and the second nucleotide corresponds to the deoxyribonucleotide within the target DNA:
rG:dT>rU:dG>rG:dA≈rG:dG>rC:dA>rU:dT>rA:dA>rC:dT>rA:dC>rA:dG>rU:dC≈rC:dC.

2. The method of claim 1, further comprising providing one or more additional sgRNAs, wherein the last 19 nucleotides of the targeting sequence of each of the one or more additional sgRNAs comprise at least one mismatch with the target DNA sequence, wherein each of the one or more additional sgRNAs provide CRISPRi or CRISPRa activity on the gene that is intermediate between that obtained using the third sgRNA and that obtained using a scrambled sgRNA providing no CRISPRi or CRISPRa activity on the gene, and wherein the mismatches with the template DNA of each of the one or more additional sgRNAs are selected according to rules (a) and (b) of claim 1.

3. The method of claim 1, wherein the target gene is a mammalian gene.

4. The method of claim 3, wherein the mammalian gene is a human gene.

5. A set of single guide RNAs (sgRNAs) for obtaining a series of discrete expression levels of a target gene using CRISPRi or CRISPRa, comprising
(i) a first sgRNA that targets the gene, wherein the last 19 nucleotides of the targeting sequence of the first sgRNA is 100% homologous to the target DNA sequence;
(ii) a second sgRNA that targets the gene, wherein the last 19 nucleotides of the targeting sequence of the second sgRNA comprises one or more mismatches with the target DNA sequence such that the CRISPRi or CRISPRa activity on the gene obtained using the second sgRNA is intermediate between that obtained using the first sgRNA and that obtained using a scrambled sgRNA providing no CRISPRi or CRISPRa activity on the gene; and
(iii) a third sgRNA that targets the gene, wherein the last 19 nucleotides of the targeting sequence of the third sgRNA comprises one or more mismatches with the target DNA sequence such that the CRISPRi or CRISPRa activity obtained using the third sgRNA is intermediate between that obtained using the second sgRNA and that obtained using a scrambled sgRNA providing no CRISPRi or CRISPRa activity on the gene;
wherein the mismatches of the second and third sgRNAs are selected according to the following rules:
(a) the CRISPRi or CRISPRa activity of the second sgRNA is designed to be greater than that of the third sgRNA based on the following positional relationships, wherein the positions correspond to the number of bases in the sgRNAs upstream from the sgRNA PAM:
−19>−18>−17>−16≈−15≈−14>−13>−12>−11>−10>−9>−8>−4>−7≈−6≈−5≈−3≈−2≈−1; or
(b) the CRISPRi or CRISPRa activity of the second sgRNA is designed to be greater than that of the third sgRNA based on the following base pair rankings of the mismatched nucleotides, wherein the first nucleotide in each pair corresponds to the ribonucleotide within the sgRNA and the second nucleotide corresponds to the deoxyribonucleotide within the target DNA:
rG:dT>rU:dG>rG:dA≈rG:dG>rC:dA>rU:dT>rA:dA>rC:dT>rA:dC>rA:dG>rU:dC≈rC:dC.

6. The set of sgRNAs of claim 5, further comprising one or more additional sgRNAs, wherein the last 19 nucleotides of the targeting sequences of each of the one or more additional sgRNAs comprise at least one mismatch with the target DNA sequence, wherein each of the one or more additional sgRNAs provide CRISPRi or CRISPRa activity on the gene that is intermediate between that obtained using the third sgRNA and a scrambled sgRNA providing no CRISPRi or CRISPRa activity on the gene, and wherein the CRISPRi or CRISPRa activity of each of the one or more additional sgRNAs on the gene is determined according to rules (a) and (b) of claim 5.

7. The set of sgRNAs of claim 6, wherein the set comprises 2, 3, 4, 5, 6, 7, 8, 9, 10, 11, 12, 13, 14, 15, 16, 17, 18, 19, 20 or more sgRNAs providing intermediate levels of CRISPRi or CRISPRa activity on the gene between that obtained using the first sgRNA and that obtained using a scrambled sgRNA providing no CRISPRi or CRISPRa activity on the gene.

8. A method of obtaining a series of discrete expression levels of a target gene in a plurality of cells, the method comprising:
contacting the plurality of cells with the set of sgRNAs of claim 5; and
contacting the plurality of cells with a nuclease-deficient sgRNA-mediated nuclease (dCas9), wherein the dCas9 comprises a dCas9 domain fused to a transcriptional modulator;
thereby generating a plurality of test cells, wherein each test cell comprises an sgRNA and the dCas9,
wherein the sgRNA present in a given test cell guides the dCas9 in the test cell to the target gene and modulates its expression level as a function of the absence or presence of one or more mismatches with the target DNA sequence according to rules (a) and (b) of claim 5.

9. The method of claim 8, wherein the transcriptional modulator is a transcriptional repressor.

10. The method of claim 9, wherein the transcriptional repressor is KRAB.

11. The method of claim 8, wherein the transcriptional modulator is a transcriptional activator.

12. The method of claim 11, wherein the transcriptional activator is VP64.

13. The method of claim 8, wherein the cells are mammalian cells.

14. The method of claim 13, wherein the cells are human cells.

15. The method of claim 8 wherein each sgRNA is encoded by an expression cassette comprising a polynucleotide encoding the sgRNA, operably linked to a promoter.

16. The method of 8, wherein the dCas9 is encoded by an expression cassette comprising a polynucleotide encoding the dCas9, operably linked to a promoter.

17. The method of claim 8, further comprising determining the relationship between the expression level of the target gene and a phenotype, comprising:
(i) determining the identity of the sgRNA present in a given test cell;
(ii) assessing the phenotype of the test cell; and
(iii) correlating the expression level of the gene targeted by the sgRNA identified in step (i) and the phenotype assessed in step (ii).

18. The method of claim 17, wherein assessing the phenotype of the cells comprises fluorescence activated cell sorting, affinity purification of the cells, measuring the transcriptomes of the cells, or measuring the growth, proliferation, and/or survival of the cells.

19. The method of claim 18, wherein the transcriptomes of the cells are measured by perturb-seq.

20. A method of determining a therapeutic window for the inhibition of a gene, the method comprising determining the relationship between the expression level of the gene and the phenotype according to the method of claim 18 for a plurality of sgRNAs targeting the gene, wherein the transcriptional modulator is a transcriptional repressor, and wherein the phenotype of the cells is assessed by measuring cell growth or survival; and further comprising:
(iv) determining the minimum level of expression of the gene that is compatible with cell growth or survival, thereby determining the lower boundary of the therapeutic window for the inhibition of the gene.
